SEQUENCE LISTING (29 sequences)

SEQ ID NO: 1 - DNA, 1471 bp, Bacillus safensis
  source 1..1471: mol_type = genomic DNA
gacgaacgctggcggcgtgcctaatacatgcaagtcgagcggacagaagggagcttgctcccggatgttagcggcggacgggtgagtaacacgtgggtaacctgcctgtaagactgggataactccgggaaaccggagctaataccggatagttccttgaaccgcatggttcaaggatgaaagacggtttcggctgtcacttacagatggacccgcggcgcattagctagttggtggggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgcgagagtaactgctcgcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggrcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggctgcragaccgcaaggtttagccaatcccataaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtg

SEQ ID NO: 2 - DNA, 1486 bp, Bacillus megaterium
  source 1..1486: mol_type = genomic DNA
gatgaacgctggcggcgtgcctaatacatgcaagtcgagcgaactrattagaagcttgcttctatgacgttagcggcggacgggtgagtaacacgtgggcaacctgcctgtaagactgggataacttcgggaaaccgaagctaataccggatasgatcttctccttcatgggagatgattgaaagatggtttcggctatcacttayagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacdagagtaactgctygtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagayagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgygaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaa

SEQ ID NO: 3 - DNA, 1559 bp, Bacillus megaterium
  source 1..1559: mol_type = genomic DNA
catttcttcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacgagagtaactgctcgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctcctttcta

SEQ ID NO: 4 - DNA, 1923 bp, Bacillus safensis
  source 1..1923: mol_type = genomic DNA
gtggcaatggaacagcaacataatagttatgatgaaaatcagatacaggtgcttgaaggattagaagctgttagaaaacgtccaggaatgtacattgggtcaaccaatgcaaaagggcttcaccatcttgtatgggaaattgtcgacaacagtattgatgaggcattagctggttattgtacagacattacagtgcaaattgaaaaagataacagcattacagtaaaagataatggccgcgggattcctgttggaattcatgagaaaatggggcgtcctgccgtagaggtcattatgactgttcttcacgctggcggtaaatttgacggcagcggttataaagtatcaggcggtctgcatggtgtaggggcatctgttgtaaatgcattatctactaccttagacgtgactgtataccgtgacgggaaaatccattatcagcagttcaaacgcggcgttccagtgggagatttagaggttattggtgaaactgatgtaaccgggacaaccactcactttgtgccagatccagaaattttcacggaaaccattgaatttgattacgatacacttgctaaccgtgttcgtgagttagccttcttaacaaaaggtgtaaacatcatcatagaagacttgcgtgaaggcaaagagcggagaaatgaatactgctacgaaggcggtattaagagctatgtagaacatttaaaccgctcaaaagaagtcgttcatgaagagcctgtgtacatcgaaggtgagaaagacggaatcacggttgaagtagcattacaatataacgattcctatacaagcaatatctattccttcgccaacaatatcaacacgtatgaaggcggaacacacgaagctggttttaaaaccggtctaacgcgtgtcatcaatgactatgctcgtaaaaatggcgtattcaaagatggggatgcgaatttaagtggtgaagatgtgcgagaaggcttaacagccattatctccatcaaacatccagaccctcaattcgaaggacaaacgaagacaaagcttgggaactcagaagcgagaaccatcacagactcccttttctctgaagcacttgagaaattcttgcttgaaaatcctgattctgcgaaaaaaattgtggaaaaaggactgatggcagctcgtgcaagaatggctgccaaaaaagctcgtgagctgacaagacgtaaaagtgcactggaagtctccagcttacctgggaaactggcggactgttcttctaaagatccttccatctctgaactctatattgtagagggagattctgcgggcggatctgctaaacaaggccgtgatcgtcacttccaagcgatcttaccgttaagagggaagatcctaaacgtagaaaaagctcgtttagataaaattttatcgaacaacgaggttcgttcaatgattacagcgttaggaactggaatcggagaagactttactttagagaaagcacgctatcacaaaattgtgatcatgacagatgccgatgtggatggagcacacattcgaacgctgctcttaacattcttctatcgctacatgcgtgaaatcattgaaaatggttatgtgtatattgcgcagccgcctttatataaagtgcagcaaggaaaacgtgtggagtacgtgtataacgataaacagctggatgagctgttaaaatcacttcctcaaacaccaaagcctggacttcagcgttataaaggtcttggagaaatgaatgctgctcagctttgggaaacaacaatggaccctgatgcgagaacacttcttcaagtcacacttgaggatgcgatcgatgctgatgagacatttgaaatgctgatgggagataaagtagaaccgagacgaaactttatcgaagaaaatgcgcaatacgtgaaaaaccttgatatttag

SEQ ID NO: 5 - DNA, 1917 bp, Bacillus megaterium
  source 1..1917: mol_type = genomic DNA
atggaacaaaaagaagtacaagcatatgaagctgatcagatacaagtattagaaggattagaagctgttcgtaaacgtccggggatgtatattggatcgacgagcgcaaagggtttacatcatcttgtatgggaaattgtagataatagtattgatgaagcgctggccggctattgcgatgaaattaatgttattatcgaaaaggataatagtattacagtcaaagataacggtcgtggaattccagttggtattcaagaaaaaatgggcagacctgctgttgaagttatcttaacggttcttcatgccggaggtaaatttggcggcggcggctataaagtatctggtggattacacggtgtaggtgcctcagttgttaacgcactttctacctctttggaagtacacgtacatcgtgacggtaaagttcattatcaaaaatatgaacgaggtgtaccggctgctgacttaaaagtagttggagaaacagataaaacaggtactgttattcaattccatccagacggtgaaatttttacagaaacgcttgaatacgattttgatacgttagctaatcgtctgcgtgagttagctttcttaaaccgcggcattaaaattacgattgaagacaaacgtgaagaagataaaagacgtgaataccattacgaaggcggaattaagtcttacgttgaacacttaaaccgttcgaaagaagtgattcacgaagagccgatctatattgaaggtaatcgagacaacatttctgtagaaattgctattcaatataacgatagctacacaagtaatttatattctttcgcaaacaacattcacacatatgaaggtggaacgcacgaagcaggatttaaaacagcgttaacgcgtgtaattaacgactatgcacgtaaaaacagcgtatttaaagatagtgacgccaatttaacgggtgaagatgttcgtgaaggaattacagctattatctctattaagcacccagatccgcagtttgaaggacaaacaaaaacaaagctgggaaatagtgaagcaagaacaattactgactctgtgtttgcagaacacttagaaacttacttgctagagaaccctattgtggcgaaaaaggtaattgaaaaaggtttaatggctgcaagagcaagaatggcagctaaaaaagctcgtgagcttacaagacgtaaaagcgcgcttgaaatttcaaacttaccgggtaaattagcagattgttcatcaaaagatccttctattagcgaactctatgtagtagagggtgactctgccggaggttcagctaagcagggaagaagccgtcatttccaagctattttgcctttgcgcggtaaaattatcaacgtagagaaagcgcgtttagataaaattttatctaataacgaaattcgtacaatcattaccgctctaggaacgggtattggtgacgattttgatatttcgaaagcccgctaccataaaattgtgattatgacagatgcagacgtagatggtgcgcatattcgtacgcttcttctaacgttcttctatcgctatatgagacagattattgagcacggatatgtgtacattgcccagccgccgctttacaaagtttcacaaggtaaaaaagtagagtacgcgtacaacgatcgtcaattagaagaggtattagcttctttccctgaaggcgcaaaaccaaaccttcagcgttacaaaggtttaggagagatgaatcctgaacaattatgggaaacaacaatggatccagaattccgtacccttcttcaggtgaacttgcaagatgcaattgaagctgatgagacatttgaaattttaatgggcgacaaagtagaaccgcgccgtaatttcattgaagaaaatgctcagtacgtaaaaaatctcgatatttaa

SEQ ID NO: 6 - DNA, 1926 bp, Bacillus megaterium
  source 1..1926: mol_type = genomic DNA
ttaaatatcgagattttttacgtactgagcattttcttcaatgaaattacggcgcggttctactttgtcgcccattaaaatttcaaatgtctcatcagcttcaattgcatcttgcaagttcacctgaagaagggtacggaattctggatccattgttgtttcccataattgttcaggattcatctctcctaaacctttgtaacgctgaaggtttggttttgcgccttcagggaaagaagctaatacctcttctaattgacgatcgttgtacgcgtactctacttttttaccttgtgaaactttgtaaagcggcggctgggcaatgtacacatatccgtgctcaataatctgtctcatatagcgatagaagaacgttagaagaagcgtacgaatatgcgcaccatctacgtctgcatctgtcataatcacaattttatggtagcgggctttcgaaatatcaaatcgtcaccaatacccgttcctagagcggtaatgattgtacgaatttcgttattagataaaaattttatctaaacgcgctttctctacgttgataattttaccgcgcaaaggcaaaatagcttggaaatgacggcttcttccctgcttagctgaacctccggcagagtcaccctctactacatagagttcgctaatagaaggatcttttgatgaacaatctgctaatttacccggtaagtttgaaatttcaagcgcgcttttacgtcttgtaagctcacgagctttttttagctgccattcttgctcttgcagccattaaacctttttcaattaccttttcgccacaatagggttctctagcaagtaagtttctaagtgttctgcaaacacagagtcagtaattgttcttgcttcactatttcccagctttgtttttgtttgtccttcaaactgcggatctgggtgcttaatagagataatagctgtaattccttcacgaacatcttcacccgttaaattggcgtcactatctttaaatacgctgtttttacgtgcatagtcgttaattacacgcgttaacgctgttttaaatcctgcttcgtgcgttccaccttcataatgtgtgaatgttgtttgcgaaagaatataaattacttgtgtagctatcgttatattgaatagcaatttctacagaaatgttgtctcgattaccttcaatatagatcggctcttcgtgaatcacttcttttcgaacggtttaagtgttcaacgtaagacttaattccgccttcgtaatggtattcacgtcttttatcttcttcacgtttgtcttcaatcgtaattttaatgccgcggtttaagaaagctaactcacgcagacgattagctaacgtatcaaaatcgtattcaagcgtttctgtaaaaatttcaccgtctggatggaattgaataacagtacctgttttatctgtttctccaactacttttaagtcagcagccggtacacctcgttcatatttttgataatgaactttaccgtcacgatgtacgtgtacttccaaagaggtagaaagtgcgttaacaactgaggcacctacaccgtgtaatccaccagatactttatagccgccgccgccaaatttacctccggcatgaagaaccgttaagataacttcaacagcaggtctgcccattttttcttgaataccaactggaattccacgaccgttatctttgactgtaatactattatccttttcgataataacattaatttcatcgcaatagccggccagcgcttcatcaatactattatctacaatttcccatacaagatgatgtaaaccctttgcgctcgtcgatccaatatacatccccggacgtttacgaacagcttctaatccttctaatacttgtatctgatcagcttcatatgcttgtacttctttttgtttccatcgtcaa

SEQ ID NO: 7 - DNA, 3441 bp, Bacillus safensis
  source 1..3441: mol_type = genomic DNA
atgtttcaagatatatccccaattgaggattttactggtaacctttctcttgaattcattgattacagcctaggggatcctaagtatcctgtagcagaatcaaaagaacgtgatgtaacttactctgctccactaagagtaaaagttcgtttaattaacaaagaaactggagaagtaaaagaccaagatgtgttcatgggtgatttcccaatcatgacagacacaggtacttttatcattaatggtgcggaacgtgtaatcgtttctcagttagtgcgttctccaagtgtatatttcagtggtaaagtagacaaaaacggtaaaaaaggttttactgcgactgtcattccaaaccgtggcgcatggttagaatacgaaactgatgcgaaggatgtagtctatgtacgcatcgatcgcacacgtaagttgccggttacggttcttttgcgtgctctcggcttcagctctgatcaagagattcttgacctcattggcgagaatgaatacttacgcaacacactggaaaaagacaatacagagaatgcggataaagcacttctcgaaatctacgagcgcctccgtcctggagaaccgccaactgttgaaaatgcgaaaagcttgctagactcccgcttcttcgatccgaagagatatgacctagcaaatgttggacgctacaagattaataagaagcttcatattaaaaatagactgttcaatcaaaaattggctgaaacgctagttgatcctgaaacaggtgaaattctagcagaaaaaggtcaaattttagacagaagagttcttgataaagttcttccatacttagaaaacggcatcggatttagaaagctttatcctaatggtggcgtagtagaagatgaagtagaacttcaatctattaagatttatgcaccgactgatcaagaaggcgagcaagtgatcaacgtgattggaaatgcatatgtagaggaagctgtgaaaaacatcacgccttctgacatcattgcgtcaatcagttacttcttcaaccttcttcatggtgtaggtgacacagatgatatcgaccaccttggtaaccgtcgtctgcgttctgtaggtgaacttctgcaaaaccaattccgtattggtttaagcagaatggagcgtgttgtccgtgaaagaatgtcaattcaagacacaaacacgatcacacctcagcagctgatcaacattcgtcctgtgatcgcttctatcaaagagttctttggtagttctcagctttctcagttcatggatcaaacaaacccgcttgctgaattgacgcacaaacgtcgtctgtcagcgcttggaccgggtggtttgacacgtgagcgtgcaggaatggaagttcgtgacgttcactactctcactatggacgtatgtgtccgattgaaacaccagagggtccaaacattggtttgatcaactctctatcttcatttgcaaaagtgaatcgcttcggatttatcgaaacaccttaccgccgggttgatcctgaaactggtaaagtgacaccgagaatcgattacttaactgctgatgaagaggataactacgtagtagcacaagcgaacgctcagttagctgatgatggttcgtttatcgatgataatatcattgctcgtttcagaggggaaaacaccgttgttcctcgaaaccgcgttgactacatggacgtttcgccaaagcaggttgtttctgcagcgacagcatgtatcccattcttagaaaacgatgactctaaccgtgctctaatgggagcgaacatgcagcgtcaggctgtgcctttgatgcagccagaatcaccgattgttggtacaggtatggagtacgtatcaggtaaagactctggtgctgctgtcatctgccgatacccaggtgttgtagaacgtgtagaagcaaaaaatatttgggttcgccgttatgaagatgttgatggacaacaagtcaaaggaaacctagacaaatacagcttgctgaaatttgtccgctctaaccaagggacttgctacaaccagcgtcctatcgtaagtgttggagatgaagtcgttaaaggagaaatccttgcagacggaccttctatggaaaaaggtgaattggctctaggacgtaacgttatggtcggcttcatgacttgggacggttacaactacgaggatgcgatcatcatgagtgagcgccttgtaaaagatgacgtctacacgtctattcatattgaagaatatgaatcagaagctcgtgatacaaagcttggaccagaagaaatcactcgtgatatcccaaacgttggggaagatgctttacgcaaccttgacgagcgcggaatcatccgtatcggtgcagaagtaaaagacggagaccttcttgtaggaaaagtaacgcctaaaggtgtaactgaactaacagctgaagaacgtctattacatgcaatcttcggtgaaaaggctcgtgaagttcgtgatacgtcactacgtgttccacacggcggcggcggaattatccacgatgtaaaagtcttcaaccgtgaagatggagatgaattacctccaggtgtaaaccagttagtccgcgtatacatcgttcagaagcgtaagatttctgaaggtgacaaaatggccggacgacatggtaacaaaggggttatctctaaaatccttccagaagaagatatgccttatcttccagacggaacaccgatcgatatcatgttgaaccctctaggggtaccatctcgtatgaacatcggtcaggtacttgagcttcatatgggtatggctgcacgttacctcggcatccacattgcgtcaccagtatttgatggtgcgcgcgaggaagatgtttgggaaacgcttgaagaagcaggtatgtcccgtgatgcgaaaacagtcctttatgacggtcgaactggtgaaccattcgacaaccgtgtatctgtcggaatcatgtacatgatcaaactggctcacatggttgacgataaacttcacgctcgttcaactggaccatactcacttgttacgcagcagccacttggcggtaaagcacagtttggtggtcagcgtttcggagagatggaagtatgggcacttgaagcttacggtgcagcatatacacttcaagagatcttaacagttaaatcggatgacgttgtgggtcgtgtgaaaacatacgaagccatcgtcaaaggggataacgtacctgaaccaggtgtccctgaatcattcaaagtgttaatcaaagaacttcaaagtttaggtatggatgtgaaaatcctatctggcgatgaagaagagatagaaatgagagatttagaagacgatgaggaaacgaagaaagcagacggattagcgttatctaatgacgaagatgctgcagacctcgcacctgtcgatcttgaacgtgacgcagtcacaaaagaatag

SEQ ID NO: 8 - DNA, 3564 bp, Bacillus megaterium
  source 1..3564: mol_type = genomic DNA
ttgacaggtcaactagttcagtatggacgccaccgccaacgcagaagttatgctcgtattagtgaagttttagagttaccgaacttaatcgagattcaaacggcttcataccaatggtttttagatgaaggtttacgagaaatgttccaagatatttctccaattgatgattttacaggtaacttatcactagaatttattgattacagcttaggtgagccaaagtattcagtaggagagtctaaagaacgtgatgtaacttatgcagcacctcttcgtgttaaggtgcggttaattaataaagaaacaggtgaagtaaaagaccaagatgtgttcatgggagatttcccattgatgaccgaaacaggtacctttgtaattaatggtgctgagcgtgttatcgtatcacagttggttcgttcaccaagtgtttactatagtggaaagcttgataaaaacggtaaaaaaggatatacagcaactgttatccctaaccgtggtgcatggctagaatatgaaactgatgcaaaagatgtagtatacgtgcgtattgatcgtactcgtaaactgccagtaacagtgctgttacgcgcgctaggtttcggttctgaccaagagattattgatttaatcggtgataatgaatacatccgtaatacgcttgaaaaagataatacggaaacaacggaaaaagcgctattagaaatctatgagcgtttacgtccgggtgaaccaccgacagttgagaatgcgaagtctctattagtatctcgcttctttgatccaaaacgatatgacttagcgaacgtaggtcgctataaaattaataaaaagcttcatatcaaacatcgcttatttaatcaaaagctagctgaaacattagttgatcctgaaacaggtgaaattattgcagaaaaaggcgcaatgattgatcgccgtctgctagatcgcttgattccgatgcttgaaggtggagtaaacttcaaaacttatagtccggttggtggagtagtagaagacgatgttacattacaatctattaagatttatgcgccaaatgatccagaaggtgaaaaagtcatcactgtatcaggcaacgcttatgtaacagaagaagttaaaaatatcacacctgctgatattttagcatcaatcagttacttctttaacttgcttcatcaagtaggagacacagatgatatcgaccacttaggtaaccgtcgtctgcgttctgtaggtgaattgttacaaaaccaattccgtatcggtttatctcgtatggaacgtgttgttcgtgaaagaatgtcaattcaagacacgaatacaatcacacctcaacaattaattaatattcgtccagttattgcggcgattaaagagttctttggaagttctcaattatcacagttcatggatcaaacgaatccattaggcgaattgacgcacaaacgtcgtctttcagctctaggacctggtggtttaacgcgtgaacgcgctggtttcgaagtgcgtgacgttcactactcccactatggccgtatgtgtccgattgaaacgccagagggtccgaatatcgggttaatcaactcactttcttcatatgcaaaagtaaacaaattcggtttcatcgaaacaccttaccgtcgtatcgatcctgaaacaggtaaagtgacagagcgaattgactacttaacagctgatgaagaagataactatgttgtagcccaagcgaacgctcgtctaggtgatgatggttcattcttagatgaaaatgtcgttgcacgtttccgtggagaaaacacggttatccgtcgcgatcgtttagactatatggatgtatcaccaaaacaagttgtatctgccgctacagcatgtatcccattcttagagaacgatgactctaaccgtgcattaatgggtgcgaacatgcaacgtcaagcagtacctttattaaatcctgaagcaccaatcgtaggtacaggtatggaatatgtatctggtaaagactctggtgcagccgtgatttgtaaataccctggcgttgtagagcgcgtagaagcaaaacaaatttttgttcgccgctatgaagaagtagacggacaaaaagttaaaggtaacttagatcaatacaaattattaaaattcgttcgttctaaccaaggtacttgttacaaccagcgtccaattgtttcagttggcgacgaagtagtaaaaggtgagatcttagccgacggtccttcaatggaaaaaggtgagcttgctttaggacgaaacgtaatggttggtttcatgacatgggatggttacaactatgaggatgccatcatcatgagtgaacgccttgtgaaagacgatgtatatacgtctgttcatattgaagaatatgaatctgagtctcgtgatacgaaacttggacctgaagaaattacgcgtgacattccaaacgtaggtgaagatgcgcttcgcaacttagatgagcgtggaatcatccgcattggtgcagaagtaaaagatggagatcttttagttggtaaagtaacgccaaaaggtgtaacagaactaacagctgaagaacgtcttctacacgctattttcggtgaaaaagctcgtgaagttcgtgatacttctcttcgtgtaccgcacggcggcggtggaattatccttgatgttaaagtcttcaaccgtgaagatggcgacgaattaccaccaggtgtaaaccaattagtccgtgtatacattgttcagaagcgtaaaatttctgaaggtgacaaaatggccggtcgtcacggtaacaaaggtgtaatttcacgtattttaccggaagaagatatgccttacctaccagacggtacgccaattgacatcatgttaaacccattaggggtaccatctcgtatgaacatcggtcaggtgctagagcttcatttaggtatggctgctcgtaagcttggcattcacgttgcgtctccagtatttgatggtgcgcgtgaggaagatgtttgggcaacaatcgaagaagctggcatgtctcgtgatgctaaaacagttctatatgatggtcgaacaggtgaaccattcgataaccgtgtatcagtaggaatcatgtacatgatcaaacttgctcacatggtagacgataaacttcacgctcgttctactggaccatactcacttgttacacaacaaccacttggtggtaaagcgcagttcggtggacagcgttttggtgagatggaagtatgggcacttgaagcatacggtgctgcttatacattacaagagatcttaacagtgaaatcagacgacgtagtaggtcgtgtgaaaacatacgaagcaattgttaaaggtgaaaacattccagaacctggcatacctgaatcgttcaaagtattaatcaaagaactacaaagtttaggtatggatgtgaagatgctttctgctgacgagcaagaaattgatatcatggactcagaagaggaccatgagcaaccaacagaatcaattattgcagataacgaagaaagcctttctgaaggacaaaaagatcctgtcacaaaagagtaa

SEQ ID NO: 9 - DNA, 3559 bp, Bacillus megaterium
  source 1..3559: mol_type = genomic DNA
ttactcttttgtgacaggatctttttgtccttcagaaaggctttcttcgttatctgcaataattgattctgttggttgctcatggtcctcttctgagtccatgatatcaatttcttgctcgtcagcagaaagcatcttcacatccatacctaaactttgtagttctttgattaatactttgaacgattcaggtatgccaggttctggaatgttttcacctttaacaattgcttcgtatgttttcacacgacctactacgtcgtctgatttcactgttaagatctcttgtaatgtataagcagcaccgtatgcttcaagtgcccatacttccatctcaccaaaacgctgtccaccgaactgcgctttaccaccaagtggttgttgtgtaacaagtgagtatggtccagtagaacgagcgtgaagtttatcgtctaccatgtgagcaagtttgatcatgtacatgattcctactgatacacggttatcgaatggttcacctgttcgaccatcatatagaactgttttagcatcacgagacatgccagcttcttcgattgttgcccaaacatcttcctcacgcgcaccatcaaatactggagacgcaacgtgaatgccaagcttacgagcagccatacctaaatgaagctctagcacctgaccgatgttcatacgagatggtacccctaatgggtttaacatgatgtcaattggcgtaccgtctggtaggtaaggcatatcttcttccggtaaaatacgtgaaattaccctttgttaccgtgacgaccggccattttgtcaccttcagaaattttacgcttctgaacaatgtatacacggactaattggtttacacctggtggtaattcgtcgccatcttcacggttgaagactttaacatcaaggataattccaccgccgccgtgcggtacacgaagagaagtatcacgaacttcacgcgctttttcaccgaaaatagcgtgtagaagacgttcttcagctgttagttctgttacaccttttggcgttactttaccaactaaaagatctccatcttttacttctgcaccaatgcggatgattccacgctcatctaagttgcgaagcgcatcttcacctacgtttggaatgtcacgcgtaatttcttcaggtccaagtttcgtatcacgagactcagattcatattcttcaatatgaacagacgtatatacatcgtctttcacaaggcgttcactcatgatgatggcatcctcatagttgtaaccatcccatgtcatgaaaccaaccattacgtttcgtcctaaagcaagctcacctttttccattgaaggaccgtcggctaagatctcaccttttactacttcgtcgccaactgaaacaattggacgctggttgtaacaagtaccttggttagaacgaacgaattttaataatttgtattgatctaagttacctttaactttttgtccgtctacttcttcatagcggcgaacaaaaatttgttttgcttctacgcgctctacaacgccagggtatttacaaatcacggctgcaccagagtctttaccagatacatattccatacctgtacctacgattggtgcttcaggatttaataaaggtactgcttgacgttgcatgttcgcacccattaatgcacggttagagtcatcgttctctaagaatgggatacatgctgtagcggcagatacaacttgttttggtgatacatccatatagtctaaacgatcgcgacggataaccgtgttttctccacggaaacgtgcaacgacattttcatctaagaatgaaccatcatcacctagacgagcgttcgcttgggctacaacatagttatcttcttcatcagctgttaagtagtcaattcgctctgtcactttacctgtttcaggatcgatacgacggtaaggtgtttcgatgaaaccgaatttgtttacttttgcataagaagaaagtgagttgattaacccgatattcggaccctctggcgtttcaatcggacacatacggccatagtgggagtagtgaacgtcacgcacttcgaaaccagcgcgttcacgcgttaaaccaccaggtcctagagctgaaagacgacgtttgtgcgtcaattcgcctaatggattcgtttgatccatgaactgtgataattgagaacttccaaagaactctttaatcgccgcaataactggacgaatattaattaattgttgaggtgtgattgtattcgtgtatgaattggacattctttcacgaacaacacgttccatacgagataaaccgatacggaattggttttgtaacaattcacctacagaacgcagacgacggttacctaagtggtcgatatcatctgtgtctcctacttgatgaagcaagttaaagaagtaactgattgatgctaaaatatcagcaggtgtgatatttttaacttcttctgttacataagcgttgcctgatacagtgatgactttttcaccttctggatcatttggcgcataaatcttaatagattgtaatgtaacatcgtcttctactactccaccaaccggactataagtttgaagtttactccaccttcaagcatcggaatcaagcgatctagcagacggcgatcaatcattgcgcctttttctgcaataatttcacctgtttcaggatcaactaatgtttcagctagcttttgattaaataagcgatgtttgatatgaagctttttattaattttatagcgacctacgttcgctaagtcatatcgttttggatcaaagaagcgagatactaatagagacttcgcattctcaactgtcggtggttcacccggacgtaaacgctcatagatttctaatagcgctttttccgttgtttccgtattatctttttcaagcgtattacggatgtattcattatcaccgattaaatcgataatctcttggtcagaaccgaaacctagcgcgcgtaacagcactgttactggcagtttacgagtacgatcaatacgcacgtatactacatcttttgcatcagtttcatattctagccatgcaccacggttagggataacattgctgtatatccttttttaccgtttttatcaagctttccactatagtaaacacttggtgaacgaaccaactgtgatacgataacacgctcagcaccattaattacaaaggtacctgtttcggtcatcaatgggaaatctcccatgaacacatcttggtcttttacttcacctgtttctttattaattaaccgcaccttaacacgaagaggtgctgcataagttacatcacgttctttagactctcctactgaatactttggctcacctaagctgtaatcaataaattctagtgataagttacctgtaaatcatcaattggagaaatatcttggaacatttctcgtaaaccttcatctaaaaaccattggtatgaagcgtttgaatctcgattaagttcggtaactctaaaacttcactaatacgagcataacttctgcgttggcggtggcgtccatactgaactagttgacctgtcaa

SEQ ID NO: 10 - DNA, 1548 bp, Bacillus amyloliquefaciens
  source 1..1548: mol_type = genomic DNA
gagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtytgaaccgcatggttcagacataaaaggtggcttyggctaccacttacagatggacccgcggcgcattagctagttggtgaggtaacggctcaccaaggcracgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttatggagccagccgccgaaggtgggacagatgattggggtgaagtcgtaacaaggtagccgtatcggaaggtgcggctggatcacctcctttcta

SEQ ID NO: 11 - DNA, 1546 bp, Bacillus licheniformis
  source 1..1546: mol_type = genomic DNA
agagtttgatcctggctcaggacgaacgctggcggcgtgcctaatacatgcaagtcgagcggaccgaygggagcttgctcccttaggtcagcggcggacgggtgagtaacacgtgggtaacctgcctgtaagactgggataactccgggaaaccggggctaataccggatgcttgattgaaccgcatggttccaatcataaaaggtggcttttagctaccacttrcagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggyaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacaaggtagccgtatcggaaggtgcggctggatcacctcctttca

SEQ ID NO: 12 - DNA, 1836 bp, Bacillus amyloliquefaciens
  source 1..1836: mol_type = genomic DNA
atgtacatcggatcaactaacagcaaaggccttcaccacttggtgtgggaaatcgtcgacaacagtattgacgaagccctggccggttattgtacagatattaacatcgagattgaaaaagataacagcattaccgttaaggacaacgggcgcggaattccggtcggtatccaggagaagatgggccgccctgcggttgaagtcatcatgaccgttctccacgccggcggtaaatttgacggaagcggatataaagtatccggcggtcttcacggtgtaggggcatctgtcgtaaacgccttgtcgactactcttgacgttacggttcatcgtgacggaaaaatccactatcaggcgtacgagcgcggtgtacctgtggccgatcttgaagtgatcggtgatactgataagaccggaacgattacgcacttcgttccggatccggaaatcttcaaagaaacaaccgtatacgactatgatctgctttcaaaccgtgtccgggaattggccttcctgacaaaaggcgtaaacatcacgattgaagacaaacgtgaaggacaagaacggaaaaacgagtaccactacgaaggcggaatcaaaagctatgttgagtacttaaaccgttccaaagaagtcgttcatgaagagccgatttatatcgaaggcgagaaagacggcataacggttgaagttgcattgcaatacaacgacagctatacaagcaatatttattctttcacgaataatatcaacacatacgaaggcggcacgcacgaggccggatttaaaaccggtctgacccgtgtcataaacgactatgcaagaagaaaagggattttcaaagaaaatgatccgaatttaagcggggatgatgtgagagaagggctgactgccattatttcaattaagcaccctgatccgcaattcgaagggcagacgaaaacgaagctcggcaactccgaagcgagaacgatcactgatacgctgttttcttctgcgctggaaacattccttcttgaaaatccggactcagcccgcaaaatcgttgaaaaaggtttaatggccgcaagagcgcggatggcagcgaaaaaagcacgggaattgacccggcgcaaaagtgcgcttgagatttccaatctgccgggcaaactggcggactgttcttctaaagatccgagcatttccgagctgtatatcgtagagggtgactctgcgggcggatcagcgaaacagggacgggaccgtcatttccaagctattctgccgctgcgcggtaagattctgaacgttgagaaagccagacttgataagattctctcaaacaatgaggtcagatcaatgatcacggccctcggaacaggaatcggagaagattttaatcttgaaaaagcgcgttatcataaagtggtcatcatgacggatgcagatgttgacggcgcccacatcagaacgcttttattaacgttcttctacagatacatgcgggaaatcatcgaaaacggctatgtctacattgcccagccgccgctttataaagtgcagcagggaaaacgggtggaatacgcttataacgataagcagcttgatgagctgttaaaagagcttccgcaatcacctaagcctggcctccagcgttataaaggtcttggagaaatgaacgcgactcagctttgggaaacgacaatggaccctgcgaccagaacgcttctgcaagtcaatcttgaagatgcaatggacgctgacgagacttttgaaatgctgatgggtgacaaagtagaaccgcggagaaacttcatagaagcaaacgccagatacgtgaaaaatcttgatatttaa

SEQ ID NO: 13 - DNA, 1920 bp, Bacillus licheniformis
  source 1..1920: mol_type = genomic DNA
gtggcaatggaacagcaaaataattacgatgaaaatcagatacaggttctagagggattggaagccgttcggaaaagacccggtatgtacatcgggtcgacaagcggcaaaggtctgcaccatttagtatgggaaattgtcgataacagcatcgatgaagcattggccggttactgcactgaaatcaatgtcgcaattgaaaaagacaacagcatcacagtaaaagacaacggacggggtatcccggtcggtattcatgagaagatgggtcgtcccgctgtggaagtcatcatgactgtcctgcacgccggaggaaagtttgacggaagcggatataaagtttcgggcggtttgcacggcgtcggtgcttctgttgttaacgccctttcaaccgagctggatgtaacggtttacagagatggaaaaatccattatcaggaatttgaacgtggcgttccgaaagctgatttgaaagtcattggagatacggaagtgacgggaacgaccacacacttcaagcctgatccggaaatattcacggaaacgactgaatacgactatgatacgctcgccactcgtgtccgcgaactcgctttcttgacaaaaggcgtcaaaatcacgatcgaagacaagcgagaaggaaaagaacgcaagaatgaatactgctatgaaggcggtattaaaagctatgttgaacacttgaaccgttcgcgggaagttattcatgaagagccggtctatattgaaggatccaaagacggcattacagtcgaggtggctcttcaatacaatgacagctatacaagcaacatttattcatttgctaacaacattcatacgtatgaaggcggaacccatgaagccggctttaagaccggtttgacgagggtcatcaatgattacgcgagaagaaacggcgtattcaaagaaagcgatccgaacttaagcggagaagacgtccgggaaggtttgacagcgattatttcaatcaagcacccggatcctcaatttgaagggcagacgaaaacaaagctcggcaactcagaagcgcgtacgataacagatgcgctattttcagaagcgcttgaaaagtttctgctagaaaacccggattcagcgaaaaaaatcgttgaaaaaggggttatggccgccagagcacggatggctgcaaagaaagcacgcgaattgacgcgcagaaaaagcgcccttgaagtgtccaatctgccggggaaacttgctgactgttcttctaaagacccgacgatttccgaactttacatcgttgagggtgactctgcgggcggatcggcaaaacagggccgcgaccgtcatttccaagcaattttgcctttgagagggaaaattttgaacgtcgaaaaagcccgcctggacaaaatattgtccaacaatgaggttcgttctatgatcaccgcccttggcaccggaatcggggaagatttcaaccttgaaaaagcccgctaccacaaagtcgtgattatgacggatgccgacgtcgatggtgcacacatcagaacattgctgctgacctttttctaccgctacatgagacagattatcgaacaaggatatgtctacatcgctcagccgcctctttacaaagtacagcaggggaaacgcgttgagtatgcgtacaatgaccagcagctcgaagaaattttaaaagagcttccgcaaaatcctaagccaggcctgcagcgctacaaaggtttgggcgagatgaatgcgacacagctttgggaaacgacgatggatccagaaacgcggacattgcttcaagtaacgctcaaagatgcgattgatgcggatgaaacctttgaaatgctgatgggcgacaaggttgaaccgcgccgcaacttcatcgaagagaatgcccgatatgtaaaaaatttggatatttaa

SEQ ID NO: 14 - DNA, 3246 bp, Bacillus amyloliquefaciens
  source 1..3246: mol_type = genomic DNA
atgggtgatttccctattatgacagataccggtacttttatcatcaacggtgcagaacgtgttatcgtatctcagcttgttcggtctccaagtgtatatttcagtggtaaagtagacaaaaacggtaaaaaaggttttaccgcgactgtcattccaaaccgtggcgcatggttagaatacgaaactgatgcgaaagatgttgtgtatgtccgcattgatcgcacacgtaagttgccggttacggttcttttgcgtgctctcggcttcggttccgaccaagagattctcgatctcattggtgagaacgaatatctccgcaatacactggataaggacaacactgaaaacagtgacaaagcgcttcttgaaatctatgagcgccttcgtcccggagagccgcctacagtagaaaacgcaaaaagcttgctggattcccgcttctttgatccgaagcgatacgatcttgcgaatgtaggacgctataaaattaataaaaagcttcatatcaagaaccgcctgtttaaccagcgccttgcagaaacactggtggatccggaaaccggtgaaattctcgctgaaaaagggcagattcttgacagaagaacacttgataaagtactgccatacttagaaaatggaatcggcttcagaaaactttatcctaacggcggcgtcgtcgaggatgaagtgatgcttcaatccattaaaatctatgctcctaccgatgcagaaggagagcagacgatcaatgtgatcggcaatgcttacatcgaagaggcgattaaaaacattacgcctgctgatattatttcttctatcagctacttcttcaacctcctgcacggagtgggcgacactgatgatatcgaccatctcggaaaccgccgtctgcgttctgtaggtgagctcctgcaaaaccaattccgtatcggtttaagccggatggaacgtgtcgtacgtgaaagaatgtctattcaagacacgaatacaattacgccgcagcagctgattaacatcagacctgttattgcgtctattaaagagttcttcggaagctcacagctttctcaattcatggatcagacgaacccgcttgctgaattgacgcacaaacgccgtctgtcagctctcggaccgggcggtttgacacgtgagcgcgcaggtatggaagtgcgtgacgttcactactctcactatggccgtatgtgtccgattgaaacgcctgagggcccgaacatcggtttgatcaactcattgtcatcatttgcgaaagtaaaccgctttggtttcattgagacgccataccgccgcgttgatcctgaaacaggaaaagtaacgcctagaatcgactacctgactgctgatgaagaggataactatgtcgtagcccaagcgaatgctaagctgagcgatgacggttctttcttggatgacagcatcgtagcgcgtttcagaggggaaaacaccgttgtagcccgcaaccgcgtggattacatggacgtatctcctaaacaggttgtatctgctgcgacagcatgtattccgttcttggaaaacgatgactcgaaccgcgccctcatgggagcgaacatgcagcgtcaggctgtgcctttgatgcagccggaagctccgatcgtcggaacgggtatggaatacgtatccggtaaagactccggtgcagccgttatttgtaaacaccctggtatcgttgaacgggtggaagcgaaaaacgtatgggtgcgccgctatgaagaaattgacggccaaaaagtaaaaggcaacctggataagtacagcttgctgaaatttgtccgctccaaccaaggtacgtgctacaaccagcgtccgatcgtcagtgtcggcgatgaagtagtcaaaggagaaatccttgctgacggaccttcaatggagcttggtgaacttgctctcggccgcaacgtaatggtcggcttcatgacatgggatggttacaactatgaggatgccatcatcatgagtgaacgccttgtgaaagatgatgtatacacatctattcacattgaagaatatgaatcagaagcacgtgatacaaagcttgggccggaagagatcacccgcgatattccaaacgtaggggaagacgcgcttcgcaatcttgatgaccgcggaattatccgtatcggtgcggaagtcaacgacggagaccttctcgtaggtaaagtaacgcctaaaggtgtaactgagcttacggctgaagaacgccttctgcatgcgatctttggagaaaaagcgcgtgaagtccgtgatacttctctccgtgtgcctcacggcggcggcggaattatccacgacgtaaaagtcttcaaccgtgaagacggcgacgaacttcctccgggagtgaaccagcttgtacgcgtatatatcgttcagaaacgtaagatttctgaaggtgataaaatggccggacgtcacggaaacaaaggggttatctcgaagattcttcctgaagaagatatgccttaccttcctgacggcacgccgatcgatatcatgcttaacccgctgggtgtaccatcacgtatgaatatcggtcaggtattagaacttcacatgggtatggctgcccgctacctcggcattcacatcgcgtcacctgtatttgacggcgcgcgtgaagaagatgtgtgggaaacacttgaagaagcaggcatgtcaagagacgctaaaacagtactttatgacggccgtacgggagaaccgtttgacaaccgtgtatctgtcggaatcatgtacatgatcaaactggcgcacatggttgatgataaacttcatgcccgttctacaggtccttactcacttgttacgcagcagcctctcggcggtaaagcccaattcggcggacagcgtttcggtgagatggaggtttgggcgcttgaagcttacggcgcagcttacacgcttcaagaaatcctgacagtgaagtccgatgacgtggtcggacgtgtgaaaacatatgaagccatcgtcaaaggcgacaatgttccagagcctggtgttccggaatcattcaaagtattgatcaaagagcttcaaagcttaggtatggacgtaaaaatcctttcaggcgatgaagaagaaatagaaatgagagatctagaagacgaggaagatgcgaaacaggctgacggccttgcattatcaggtgatgaagcgccggaagaaacagcatctccagacgttgaacgtgacgcagtaacgaaagagtag

SEQ ID NO: 15 - DNA, 3441 bp, Bacillus licheniformis
  source 1..3441: mol_type = genomic DNA
atgtttcaagacatatcgccaattgaggatttcactggtaacctctctctggaatttatcgactacagcttgggcgagcctaagtatccggtagaagaatcaaaagagcgggatgtgacctattcagctccgctgcgggttaaagtccgcttaatcaacaaagaaaccggcgaagtaaaagatcaggatgtcttcatgggcgatttccctattatgacagacactggaaccttcattatcaacggtgcagaacgggtcatcgtatctcagctcgttcgttctccaagtgtatattttagtggtaaagtagacaaaaacggtaagaaaggttttaccgcgactgtcattccaaaccgtggcgcatggttagaatacgagactgatgcgaaagatgttgtttacgtacgcatcgatcgcacacgtaagttgccggttacggttcttttgcgtgctcttggcttcggatctgaccaagagatcattgatctcatcggtgaaaacgagtatctgcgcaatacgcttgataaagataatacggaaaacaccgataaagcgcttcttgaaatctacgagcgtcttcgtccaggggagccgcctaccgtagaaaacgcaaaaagcctgcttgattcaaggttctttgatccgaaaagatatgaccttgcgagtgtaggacgttataaaattaataaaaagcttcacatcaaaaaccgactttttaatcagcggcttgctgaaacgctagtcgaccctgaaacaggcgaaatccttgccgaaaaaggagcgattttagacagaagaacgcttgataaagttctgccgtaccttgaaaacggaatcggttttaaaaagctttatccgaacggcggagttgtcgaagacgaagtaacgcttcagtctatcaaaatctatgctccgacagaccaagaaggggagcagacaatcaatgtgattggaaatgcttatatcgaagaaggcgttaagaatattacaccttctgatattatcgcttccatcagctatttctttaacctgcttcacggagtgggcgataccgacgatatcgaccatctaggaaaccgccgtctccgttcagtgggagagcttctgcaaaaccaattccgtattggtttaagcagaatggagcgcgttgttcgtgaaagaatgtctattcaagatacaaacacgatcacgccgcagcagctgatcaatattcgccctgtcatcgcatcaatcaaagagtttttcggaagctcgcagctttctcagtttatggatcagacgaatccgcttgctgagctgacgcataagcgccgtctgtcagcgctcggaccgggcggtttgactcgtgagcgcgccggaatggaagtccgtgacgttcactattcacactacggccggatgtgtccgattgaaacccctgagggtccaaacatcggcttgatcaactcgctttcttcattcgcgaaagtgaaccgtttcggcttcatcgaaacgccgtatcgccgcgtcgatcctgaaactggaaaagtaacgccgagaatcgattacttgacagctgatgaagaagacaactatgtcgttgcccaggcaaacgcacgcctgaatgatgacggttcttttgtggatgacagcatcgtcgcccgtttcagaggggagaacaccgttgttccgaaagaccgcgttgactatatggacgtttcgcctaaacaggttgtctctgccgcgactgcatgtattcctttcttggaaaacgatgactcaaaccgcgcccttatgggagcgaacatgcaacgtcaggccgtacctcttatgcagcctgaatcgccgatcgtcggaaccgggatggaatatgtatctgcgaaagactccggtgccgctgttatttgccgccaccctggaatcgttgaacgggtggaagcgaagaacatctgggtgcgccgctatgaagaagtcgacggccagaaagtcaaagggaaccttgataaatacagcctgctgaagtttgtccgttccaaccagggaacttgctacaaccagcgtccgatcgtaagcgtcggtgatgaggttgaaaaaggtgaaattttagctgacggtccgtccatggaaaaaggtgagcttgcccttggacgcaacgtcatggtcggctttatgacatgggatggctacaactatgaggatgccatcatcatgagcgaacgccttgtaaaagacgacgtatacacgtctattcatattgaagaatacgaatcagaggcccgggatacaaaactcggacctgaagaaatcactcgcgatattccgaacgtcggtgaggacgctcttcgcaatctcgatgaacgcggaattatccgtgtcggtgctgaagtaaaagacggagatcttcttgttggtaaagtaacccctaaaggtgttacagagcttacggcggaagaacgcctgcttcatgccatcttcggggaaaaagcgcgtgaagtgcgtgatacgtcgctgcgtgtacctcacggaggcggcggtatcatccttgatgtaaaagtgttcaaccgcgaagacggagacgaactgcctccgggcgttaaccagctcgtccgcgtctacatcgttcagaagcgtaaaatttctgaaggggacaaaatggccggacgccacggtaacaaaggtgttatttcgaaaattcttccggaggaagatatgccgtatctgcctgacggaacgccaattgacatcatgttaaacccgctgggcgtaccatcgcgtatgaacatcgggcaggtgttggagctgcaccttggtatggctgcacgccgcctcggtctgcatgtcgcgtcacctgtatttgacggtgcccgcgaagaagatgtgtgggaaacccttgaagaagccggcatgtcaagagacgcaaaaaccgtcctttacgacggccgaactggggagccgtttgacaaccgggtttctgtcggcatcatgtacatgatcaaactggcacacatggttgacgacaaattgcacgcacgttcaaccggtccttactcactcgttacccagcagcctctcggaggtaaagcgcagttcggcggacagcgttttggagagatggaagtttgggcgcttgaagcttatggtgcagcatatacgctacaagagatcctgactgttaaatcggatgatgtcgtaggccgtgtgaaaacatatgaagccatcgtaaaaggcgacaatgttccagaacctggtgttccggaatcgttcaaagtattgatcaaagagcttcaaagcttaggtatggacgtcaaaattctatcaagcgacgaagaagaaatcgaaatgagagacttggaagacgacgaagacgcgaaacaaaacgaagggctttctctgccgaatgatgaagagtccgaagagttagtttctgctgacgcagaacgcgatgtcgttacaaaagaataa

SEQ ID NO: 16 - DNA, 1559 bp, Bacillus megaterium
  source 1..1559: mol_type = genomic DNA
catttcttcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctcctttcta

SEQ ID NO: 17 - DNA, 1567 bp, Paenibacillus borealis
  source 1..1567: mol_type = genomic DNA
ttcaatacaaattggagagtttgatcctggctcaggacgaacgctggcggcgtgcctaatacatgcaagtcgagcggagttatgatggagcttgctcctgattaacttagcggcggacgggtgagtaacacgtaggcaacctgccctcaagactgggataactaccggaaacggtagctaataccggataatttctttgttctcctgaagagagaatgaaaggcggagcaatctgccacttgaggatgggcctgcggcgcattagctagttggtgaggtaacggctcaccaaggcgacgatgcgtagccgacctgagagggtgaacggccacactgggactgagacacggcccagactcctacgggaggcagcagtagggaatcttccgcaatgggcgaaagcctgacggagcaacgccgcgtgagtgatgaaggttttcggatcgtaaagctctgttgccagggaagaacgtccggtagagtaactgctaccggagtgacggtacctgagaagaaagccccggctaactacgtgccagcagccgcggtaatacgtagggggcaagcgttgtccggaattattgggcgtaaagcgcgcgcaggcggcgatttaagtctggtgtttaaaccttgggctcaacctggggtcgcactggaaactggatcgcttgagtacagaagaggaaagtggaattccacgtgtagcggtgaaatgcgtagatatgtggaggaacaccagtggcgaaggcgactttctgggctgtaactgacgctgaggcgcgaaagcgtggggagcaaacaggattagataccctggtagtccacgccgtaaacgatgagtgctaggtgttaggggtttcgatacccttggtgccgaagttaacacagtaagcactccgcctggggagtacggtcgcaagactgaaactcaaaggaattgacggggacccgcacaagcagtggagtatgtggtttaattcgaagcaacgcgaagaaccttaccaggtcttgacatcccgatgaaagcattagagatagtgcccctcttcggagcatcggagacaggtggtgcatggttgtcgtcagctcgtgtcgtgagatgttgggttaagtcccgcaacgagcgcaacccttgactttagttgccagcaggttaagctgggcactctagagtgactgccggtgacaaaccggaggaaggtggggatgacgtcaaatcatcatgccccttatgacctgggctacacacgtactacaatggccggtacaacgggaagcgaagccgcgaggtggagccaatcccagcaaagccggtctcagttcggattgcaggctgcaactcgcctgcatgaagtcggaattgctagtaatcgcggatcagcatgccgcggtgaatacgttcccgggtcttgtacacaccgcccgtcacaccacgagagtttacaacacccgaagtcggtggggtaacccgcaagggagccagccgccgaaggtggggtagatgattggggtgaagtcgtaacaaggtagccgtatcggaaggtgcggctggatcacctcctttcta

SEQ ID NO: 18 - DNA, 1557 bp, Paenibacillus sonchi
  source 1..1557: mol_type = genomic DNA
taccaattggagagtttgatcctggctcaggacgaacgctggcggcgtgcctaatacatgcaagtcgagcggagtttatccttcggggtaagcttagcggcggacgggtgagtaacacgtaggcaacctaccctctagactgggataactaccggaaacggtagctaataccggataattccttgatccacatgggctaaggatgaaaggcggagcaatctgctgctagaggatgggcctgcggcgcattagctagttggtggggtaacggcctaccaaggcgacgatgcgtagccgacctgagagggtgaacggccacactgggactgagacacggcccagactcctacgggaggcagcagtagggaatcttccgcaatgggcgaaagcctgacggagcaacgccgcgtgagtgatgaaggttttcggatcgtaaagctctgttgccagggaagaacgtccggtagagtaactgctatcggagtgacggtacctgagaagaaagccccggctaactacgtgccagcagccgcggtaatacgtagggggcaagcgttgtccggaattattgggcgtaaagcgcgcgcaggcggctgcttaagtctggtgtttaaaccttgggctcaacctggggtcgcactggaaactgggcagcttgagtacagaagaggaaagtggaattccacgtgtagcggtgaaatgcgtagagatgtggaggaacaccagtggcgaaggcgactttctgggctgtaactgacgctgaggcgcgaaagcgtggggagcaaacaggattagataccctggtagtccacgccgtaaacgatgagtgctaggtgttaggggtttcgatacccttggtgccgaagttaacacagtaagcactccgcctggggagtacggtcgcaagactgaaactcaaaggaattgacggggacccgcacaagcagtggagtatgtggtttaattcgaagcaacgcgaagaaccttaccaggtcttgacatccaactaacgaagcagagatgcatcaggtgcccttcggggaaagttgagacaggtggtgcatggttgtcgtcagctcgtgtcgtgagatgttgggttaagtcccgcaacgagcgcaacccttgactttagttgccagcaggtgaagctgggcactctagagtgactgccggtgacaaaccggaggaaggtggggatgacgtcaaatcatcatgccccttatgacctgggctacacacgtactacaatggccggtacaacgggaagcgaagccgcgaggtggagccaatcccagcaaagccggtctcagttcggattgcaggctgcaactcgcctgcatgaagtcggaattgctagtaatcgcggatcagcatgccgcggtgaatacgttcccgggtcttgtacacaccgcccgtcacaccacgagagtttacaacacccgaagtcggtggggtaacccgcaagggggccagccgccgaaggtggggtagatgattggggtgaagtcgtaacaaggtagccgtatcggaaggtgcggctggatcacctcctttcta

SEQ ID NO: 19 - DNA, 1559 bp, Bacillus megaterium
  source 1..1559: mol_type = genomic DNA
catttcttcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacgagagtaactgctcgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcattaagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctcctttcta

SEQ ID NO: 20 - DNA, 1923 bp, Bacillus megaterium
  source 1..1923: mol_type = genomic DNA
ttgacgatggaacaaaaagaagtacaagcatatgaagctgatcagatacaagtattagaaggattagaagctgttcgtaaacgtccggggatgtatattggatcgacgagcgcaaagggtctacatcatcttgtatgggaaattgtagataatagtattgatgaagcgctggccggctattgcgatgaaattaatgttattatcgaaaaggataatagtattacagtcaaagataacggtcgtggaattccggttggtattcaagaaaaaatgggcagacctgccgttgaagttatcttaacggttcttcatgccggaggtaaatttggcggcggcggctataaagtatctggtggattacacggtgtaggtgcctcagttgttaatgcactttctacctctttggaagtgcacgtacatcgtgacggtaaagttcattatcaaaaatatgaacgaggtgtaccggctgctgacttaaaagtagttggagaaacagataaaacaggtactgttattcaattccgtccagacggtgaaatttttacagaaacgcttgaatacgattttgatacgttagctaatcgtctgcgtgagttagctttcttaaatcgtggcattaaaattacgattgaagacaaacgtgaagaagataaaagacgtgagtatcactatgaaggcggaattaagtcttacgttgaacacttaaaccgttcgaaagaagtgattcacgaagagccaatctatattgaaggtaatcgagacaacatttctgtagaaattgctattcaatataacgatagctatacaagtaatttatattcttttgcaaacaatattcacacatatgaaggtggaacgcacgaagcaggatttaaaacagcgttaacgcgtgtaattaacgactatgcacgtaaaaacagcgtatttaaagacagtgacgccaatctaacaggtgaagatgttcgtgaaggaattacagctatcatctctattaagcacccagatccgcagttcgaaggacaaacaaaaacaaagctgggaaatagtgaagcaagaacaattactgactctgtgtttgcagaacacttagaaacttacttgctagagaaccctattgtggcgaaaaaggtaattgaaaaaggtttaatggctgcaagagcaagaatggcagctaaaaaagctcgtgagcttacacgccgtaaaagcgcgcttgaaatttcaaacttaccgggtaaattagcagattgttcatcaaaagatccttctattagcgaactctatgtagtagagggtgactctgccggaggttcagctaagcagggaagaagccgtcatttccaagctattttgcctttacgtggtaaaattatcaacgtagagaaagcgcgtttagataaaattttatctaataacgaaattcgtacaatcattaccgctctaggaacgggtattggtgacgattttgatatttcgaaagcccgctaccataaaattgtgattatgacagatgcagacgtagacggtgcgcatattcgtacgcttcttctaacgttcttctatcgctatatgagacagattattgagcacggatatgtgtacattgctcagccgcctctttacaaagtttcacaaggtaaaaaagtggagtacgcgtacaacgatcgtcaattagaagaggtattagcttctttccctgaaggcgcaaaaccaaaccttcagcgttacaaaggtttaggagagatgaatcctgaacaattatgggaaacaacaatggatccagagttccgtacccttcttcaggtgaacttgcaagatgcaattgaagctgatgagacatttgaaattttaatgggcgacaaagtagaaccacgccgtaatttcattgaagaaaatgctcagtacgtaaaaaatcttgatatttaa

SEQ ID NO: 21 - DNA, 1911 bp, Paenibacillus borealis
  source 1..1911: mol_type = genomic DNA
atgtcaatgaatcaaccgacatatgatgagagccagattcaggtactggaagggctggaagcggttcggaaacgtcccggcatgtacattggctccactagcgccaaaggtctgcatcatttggtctgggaggtcgtcgataatagtatcgatgaggcgctggcaggtttttgcgaccggattcaagtgatgattcatgaagataacagcgtgactgttatcgataacggacggggtattcctgtcggagagaacgagaagctgaagaaatccacgcttgaagttgtaatgactgtcctgcatgcaggcggtaaattcggcggcgggggatataaggtttccggcggtctgcacggtgtggggatctctgttgtgaatgcattgtccgagaaggtagttgtaaaggttaaacgtgacggacacgtctaccagcaggaatacagacggggtgcgccgcagtatgatatcagggtaatcggggataccgatgagactggaaccaccaccaccttccatccggatcctgagatttttaccgaaacgactatttttgaatattcaactctgcttacccgtatccgcgagctcgccttcttgaataagggtatcgagctgtccctgctggatgaacggaccggagtctccaatacatttaagtatgatggcggtattgtggagtatgtgaagtaccttaacgagaaaaaagaagccctgcacgaggacccgatctacgtggaaggctcacgggatatgatcgcggttgaagtggctctccaatataacgattcatactcagagaacatctattcctttgccaacaatatcaacactcatgaaggcggtacgcatgaatccggcttcaagagcgccttgacacgtattatcaatgattatgcccgtaaggctggcgttatcaaggacagcagccagaatctgaccggagatgatgtacgtgaaggcttgacggcgattatttccgtcaaaattcccgaaccgcagttcgaaggccagaccaagaccaagcttgggaacagcgaagtccgcgggattgtggagtccctgttcggtgagaagctgcaggaattcctggaagagaaccctgctgtgtcccgccgcgtgctggagaagtcgctgcaggcttcacgtgcacgtgaagccgcgcgcaaagcccgtgaactgacccgccgcaagagtgcgcttgaagtcagtgccctgccgggtaagctggccgactgctcctccaaggatgcatcgatcagtgaactatacattgtcgaaggtgactctgcgggtggatcggccaagcaaggccgggaccgtcatttccaggccatcctgccgctgcgcggtaagatcctcaatgtagagaaggctcggcttgaccggatcttgtccaatgcggaaatacgggccattatcacagcgctcggaaccgggatcagcgatgatttcgacctctctaaggcacgttaccacaaggttgtcatcatgaccgatgcggatgttgacggtgcccacatcagaacactgctgctgactttcttttaccgctacatgcggaagattgtcgatgccgggtatatcttcatcgcccagccgccgctgttcaagattgaacgcaataaggttatccgctatgcgcagaccgagaaggaacgcgatgagattatcgcctccttcggtgagaacgtgaaggttaacgttcagcgctacaaaggtttgggtgagatgaatgctgcccagctctgggatacaacgatggatcctgagagccgcactatgctgcaggtaacgattgaggatgctatgctcgctgacagtatctttgatacacttatgggcgataacgtccagccaagatatgaatttatccaggagcacgcccattccgtcaaaaatctcgatatttag

SEQ ID NO: 22 - DNA, 1911 bp, Paenibacillus sonchi
  source 1..1911: mol_type = genomic DNA
atgtcaatgaatcaaccgacatatgatgagagccagattcaggtactggaagggctggaggcagttcggaaacgtcccggcatgtacatcggttctaccagcgccaaaggtctgcatcatttggtctgggaggttgtcgataacagtatcgatgaggcgctggcaggctattgcgaccggattcaagtgaagattcatgaggataacagtgttacagtaatagataatggacggggtatccctgtcggtgagaatgtgaagctgaagaagtcgacgctcgaagtcgtcatgacggtcctgcatgcagggggaaaattcggcggcggcggatataaagtatcaggcggtttgcacggtgtagggatctccgtcgtaaatgccttgtccgagaaggttgttgtgaacgtcaagcgtgacggccacatctaccagcaggaatatagacgcggtgcgccgcagtatgatatcaagatcgttggggataccgatgagacagggacaaccacaacctttcttccggatccggagattttcaccgaaacgacagtgtttgaatatacgacattgcttacacgcattcgcgaattagcctttcttaacaagggtattgagctttcgctgctggatgaacgcaccggtgtgtcgaatgttttcaaatacgagggcggtatcgttgagtatgtaaaatatctgaacgagaaaaaagaagcgctgcacgaagaaccaatctacgtagaaggctcccgtgatatgattgcggtggaagtggctctgcagtataacgattcctatacagagaacatttactcttttgccaataatattaatacccatgagggcgggacccatgaatccggcttcaaaagcgcattaacgcggatcatcaatgactatgcacgtaaaacaggagttattaaggacagcagctccaaccttaccggtgatgatgtgcgtgaagggctgacggcgattatttccgttaagattcctgagccgcaatttgaaggccaaaccaagaccaagctgggcaacagtgaagtccgcggtgttgtagaatctttattcggagagaagctgcaggagttcctggaagagaatccggcggtgtcccggcgtgtgctggagaaatccatgcaggcttcccgtgcccgtgaagcggcccgcaaggcccgtgaactgacccgccgcaaaagtgcacttgaagtaagcgccctgccggggaagctggctgactgctcctccaaggatgcctccatcagcgagctgtacatcgtcgaaggggactccgcaggcggttctgccaagcagggccgtgaccgtcatttccaggcaattctgccgctgcgcggcaagatactgaacgttgaaaaagcgcggcttgaccgtatattgtccaatgctgaaatccgggcgattatcaccgctctgggaaccggtatcagtgatgatttcgatctgtccaaagcccgttaccataaggttgttatcatgaccgatgcggatgtcgacggtgcccatatcagaacactgctgctgaccttcttctaccgctacatgcggaagattatcgaagcgggctatatttacatcgcccagccgccgctgtttaagattgagcgcaataaggtcgtccgctatgctcagaccgagaaggaacgtgatgagattattgccagcttcggcgagaatgttaaagtcaacgttcaacggtataaaggtcttggggaaatgaatgcggcacagctgtgggatacgactatggaccctgagagccgtatgatgctgcaggtcaccatcgaggatgcgattctcgctgacggaatctttgatacgctgatgggcgacaatgtccagccacgatatgaattcattcaggagaatgcgaagtacgttcggaacctggatttctaa

SEQ ID NO: 23 - DNA, 1919 bp, Bacillus megaterium
  source 1..1919: mol_type = genomic DNA
ttaaatatcaagattttttacgtactgagcattttcttcaatgaaattacggcgtggttctactttgtcgcccattaaaatttcaaatgtctcatcagcttcaattgcatcttgcaagttcacctgaagaagggtacggaactctggatccattgttgtttcccataattgttcaggattcatctctcctaaacctttgtaacgctgaaggtttggttttgcgccttcagggaaagaagctaatacctcttctaattgacgatcgttgtacgcgtactccacttttttaccttgtgaaactttgtaaagaggcggctgagcaatgtacacatatccgtgctcaataatctgtctcatatagcgatagaagaacgttagaagaagcgtacgaatatgcgcaccgtctacgtctgcatctgtcataatcacaattttatggtagcgggctttcgaaatatcaaatcgtcacaatccgtccgttcctagagcggtatgattgtacgaatttcgttattagataaaatttatctaaacgcgctttctctacgttgataattttaccacgtaaaggcaaaatagcttggaaatgacggcttcttccctgcttagctgaacctccggcagagtcaccctctactacatagagttcgctaatagaaggatcttttgatgaacaatctgctaatttacccggtaagtttgaaatttcaagcgcgcttttacggcgtgtaagctcacgagcttttttagctgccattcttgctcttgcagccattaaacctttttcaattacctttttcgccacaatagggttctctagcaagtaagtttctaagtgttctgcaaacacagagtcagtaattgttcttgcttcactatttcccagctttgtttttgtttgtccttcgaactgcggatctgggtgcttaatagagatgatagctgtaattccttcacgaacatcttcacctgttagattggcgtcactgtctttaaatacgctgtttttacgtgcatagtcgttaattacacgcgttaacgctgttttaaatcctgcttcgtgcgttccaccttcatatgtgtgaatattgtttgcaaaagaatataaattacttgtatagctatcgttatattgaatagcaatttctacagaaatgttgtctcgattaccttcaatatagattggctcttcgtgaatcacttctttcgaacggtttaagtgttcaacgtaagacttaattccaccttcatagtgatactcacgtcttttatcttcttcacgtttgtcttcaatcgtaattttaatgccgcgatttaagaaagctaactcacgcagacgattagctaacgtatcaaaatcgtattcaagcgtttctgtaaaaatttcaccgtctggacggaattgaataacagtacctgtttatctgtttctccaactacttttaagtcagcagccggtacacctcgttcatatttttgataatgaactttaccgtcacgatgtacgtgcacttccaaagaggtagaaagtgcattaacaactgaggcacctacaccgtgtaatccaccagatactttatagccgccgccaccaaatttacctccggcatgaagaaccgttaagataacttcaacggcaggtctgccattttttcttgaataccaaccggaattccacgaccgttatctttgactgtaatactattatccttttcgataataacattaatttcatcgcaatagccggccagcgcttcatcaatactattatctacaatttcccatacaagatgatgtagaccctttgcgctcgtcgatccaatatacatccccggacgtttacgaacagcttctaatccttctaatacttgtatctgatcagcttcatatgcttgtacttctttttgttccatcgtcaa

SEQ ID NO: 24 - DNA, 3564 bp, Bacillus megaterium
  source 1..3564: mol_type = genomic DNA
ttgacaggtcaactagttcagtatggacgccaccgccaacgcagaagttatgctcgtattagtgaagttttagagttaccgaacttaatcgagattcaaacggcttcataccaatggtttttagatgaaggtttacgagaaatgttccaagatatttctccaattgatgattttacaggtaacttatcactagaatttattgattacagcttaggtgagccaaagtattcagtaggagagtctaaagaacgtgatgtaacttatgcagcacctcttcgtgttaaggtgcggttaattaataaagaaacaggtgaagtaaaagaccaagatgtgttcatgggagatttcccattgatgaccgaaacaggtacctttgtaattaacggtgctgagcgtgttatcgtatcacagttggttcgttcaccaagtgtttactatagtggaaagcttgataaaaacggtaaaaaaggatatacagcaactgttatccctaaccgtggtgcatggctagaatatgaaactgatgcaaaagatgtagtatacgtgcgtattgatcgtactcgtaaactgccagtaacagtgctgttacgcgcgctaggtttcggttctgaccaagagattatcgatttaatcggtgataatgaatacatccgtaatacgcttgaaaaagataatacggaaacaacggaaaaagcgctattagaaatctatgagcgtttacgtccgggtgaaccaccgacagttgagaatgcgaagtctctattagtatctcgcttctttgatccaaaacgatatgacttagcgaacgtaggtcgctataaaattaataaaaagcttcatatcaaacatcgcttatttaatcaaaagctagctgaaacattagttgatcctgaaacaggcgaaattattgcagaaaaaggcgcaatgattgaccgccgtctgctagatcgcttgattccgatgcttgaaggtggagtaaacttcaaaacttatagtccggttggtggagtagtagaagacgatgttacattacaatctattaagatttatgcgccaaatgatccagaaggtgaaaaaatcatcactgtatcaggtaacgcatatgtaacagaagaagttaaaaatatcacacctgctgatattttagcatcaatcagttacttctttaacttgcttcatcaagtaggagacacagatgatatcgaccacttaggtaaccgtcgtctgcgttctgtaggtgaattgttacaaaaccaattccgtatcggtttatctcgtatggaacgtgttgttcgtgaaagaatgtcaattcaagacacgaatacaatcacacctcaacaattaattaatattcgcccagttattgcggcgattaaagagttctttggaagttctcaattatcacagttcatggatcaaacgaatccattaggcgaattgacgcacaaacgtcgtctttcagctctaggacctggtggtttaacgcgtgagcgcgctggtttcgaagtgcgtgacgttcactactcccactatggccgtatgtgcccgattgaaacaccagagggtccgaatatcgggttaatcaactcactatcttcttatgcaaaagtaaacaaattcggtttcatcgaaacaccttaccgtcgtatcgatcctgaaacaggtaaagtgacagagcgaattgactacttaacagctgatgaagaagataactatgttgtagcccaagcgaacgctcgtctaggtgatgatggttcattcttagatgaaaatgtcgttgcacgtttccgtggagaaaacacggttatccgtcgcgatcgtttagactatatggatgtatcaccaaaacaagttgtatctgccgctacagcatgtatcccattcttagagaacgatgactctaaccgtgcattaatgggtgcgaacatgcaacgtcaagcagtacctttattaaatcctgaagcaccaatcgtaggtacaggtatggaatacgtatctggtaaagactctggtgcagccgtgatttgtaaatatcctggcgttgtagagcgcgtagaagcaaaacaaatttttgttcgccgctatgaagaagtagacggacaaaaagttaaaggtaacttagatcaatacaaattattaaaatttgttcgttctaaccaaggtacttgttacaaccagcgtccaattgtttcagttggcgacgaagtagtaaaaggtgagatcttagccgacggtccttcaatggaaaaaggtgagcttgctttaggacgaaacgtaatggttggtttcatgacatgggatggttacaactatgaggatgccatcatcatgagtgaacgccttgtgaaagacgatgtatatacgtctgttcatattgaagaatatgaatctgagtctcgtgatacgaagcttggacctgaagaaattacgcgtgacattccaaacgtaggtgaagatgcgcttcgcaacttagatgagcgtggaatcatccgcattggtgcagaagtaaaagacggagatcttttagttggtaaagtaacgccaaaaggtgtaacagaactaacagctgaagaacgtcttctacacgctattttcggtgaaaaagcgcgtgaagttcgtgatacttctcttcgtgtaccgcacggcggcggtggaatcattcttgatgttaaagtcttcaaccgtgaagatggggacgaattaccaccaggtgtaaaccaattagtccgtgtatatattgttcagaagcgtaaaatttctgaaggtgacaaaatggccggtcgtcacggtaacaaaggtgtaatttcacgtattttacctgaagaagatatgccttacctaccagacggtacgccaattgacatcatgttaaacccattaggggtaccatctcgtatgaacatcggtcaggtgctagagcttcatttaggtatggctgctcgtaagcttggcattcacgttgcgtctccagtatttgatggtgcgcgtgaggaagatgtttgggcaacaatcgaagaagctggcatgtctcgtgatgctaaaacagttctatatgatggtcgaacaggtgaaccattcgataaccgtgtatcagtaggaatcatgtacatgatcaaacttgctcacatggtagacgataaacttcacgctcgttctactggaccatactcacttgttacacaacaaccacttggtggtaaagcgcagttcggtggacagcgttttggtgagatggaggtatgggcacttgaagcatacggtgctgcttacacattacaagagatcttaacagtgaaatcagatgacgtagtaggtcgtgtgaaaacatacgaagcaattgttaaaggtgaaaacattccagaacctggcatacctgaatcgttcaaagtattaattaaagaactacaaagtttaggtatggatgtgaagatgctttctgctgacgagcaagaaattgatatcatggactcagaagaggaccatgagcaaccaacagaatcaattattgcagataacgaagaaagcctttctgaaggacaaaaagatcctgtcacaaaagagtaa

SEQ ID NO: 25 - DNA, 3417 bp, Paenibacillus borealis
  source 1..3417: mol_type = genomic DNA
ttgcgggaaatgttccaggacatctcgccgatccaggatttcacaggtaatttggtactagagttcattgattacagcctgggtgaaccgaagtatacggttgacgacgctaaagagcgggacgtaacatatgcggctcctctgcgtgtgaaggtgcgtctcatcaataaggagaccggtgaggtcaaagagcaggaagtgttcatgggagatttccctctgatgacggagaccggcacttttattatcaatggtgcggaacgggttattgtcagccagttggttcgctctccaagcgtctatttcagcacgaaagtggataagaacggcaaaaaaacctacaccgccacagtaattccgaatcgcggagcttggctggagctggagaccgacgctaaggacatcatgtatgtccgtatcgaccggactcgtaagatcccggttaccgtgcttctgcgtgctctaggcttcggcagtgatgctgaaattctggaactgcttggtaatgatgaatatattcgcaatacgctggataaagacaacacggactctacggagaaggcgcttatcgaaatttacgagcgtctgcgtccgggcgaaccaccgacacttgacaatgccaagagccttttggtcgcacgtttctttgatccgaaacgttatgatttggccaatgtaggccgttacaaaatcaacaaaaagctgcatattaaaaatcgtctgttcaatcagcgtctggcacaacctttggtggatgagtctactggagaaatcctggcagaatccggccaaatggttgaccgccgcctgcttgatgagctgattccttattttgagaagaacgtagctgccaagaactaccgtgtaaccggtggggttatggacagcgaagatattccgcttcagacgattgatgtgttctcgccaattgaagaaggccggattatcaaactgatcgccaatggcaacattgacaagtcggtcaagcatattactcaggctgatattatatcctcaatcagctactttattaatctgctgcacggtatcggcaacactgatgatatcgaccacttgggtaaccgccgtctgcgttctgtcggcgaactgctgcagaatcagttccgtatcggtctgtcccgcatggaacgcgttgtccgcgagagaatgtcgattcaggatgccaatgcgatcacaccgcaggcgctgatcaacatccgcccggttatcgcgtcgattaaagagttcttcggtagttcacagctgtcccagttcatggatcagacgaacccgcttgctgaacttacgcacaaacgccgtctatcggcactcggacccggcggtttgacccgtgaacgcgcaggctttgaagtccgcgacgtccatcacagtcactatggccgtatgtgtccaatcgaaacaccggaaggtccgaatatcggtctgatcaactccttgtccacctttgcccgcatcaatgaatacggctttatcgaagcaccgtatcgttgggtggatccaaagacaggcaaagtcactgagcaaattgattatctgactgccgatgaagaagataactatgtagttgcacaggcaaatgtactgatcgatgagaatggctccttcaaggaagaccaggttatcgttcgttataacaaagattcagacaacatcactacaatgccaagtaaccgtgttgactacatggacgtttcgcctaaacaggttgtatcagtagctacggcgctcattccgttccttgagaacgatgactccaaccgcgcgctgatgggatcgaacatgcagcgtcaggctgttccgcttctcattccgaaggctccgcttgtaggaacagggatggaacataaatccgctaaggactcgggcgtatgtattgtctccaaatatgacggtattatcgaacgctcctctgccaatgaaatctggctgcgccgtgttgaggcagttgaaggcaaagaagttaaaggcgatatcgttaaatataaattacacaaatttatgcgttcgaaccagggtacctgcattaatcagcgtccgctagccaaacgcggcgacgttgttaagaaaggtgatatccttgcagacggaccatctacggaaatgggcgaacttgctcttggccgtaacgtagttgttgcgttcatgacttgggaaggctacaactacgaggatgcgatcctgctgagtgagaagctggttaaggaagatgtatacacttcgatccatatcgaggaatacgaatccgaagcccgtgacacgaagcttggacctgaagaaatcacgcgtgatattccaaacgtaggggaagaagcgcttcgcaatctcgatgagcgcggaattatccggatcggtgcggaaatcaatgccggcgacattctggtaggtaaagttactccgaagggcgtaaccgagctgaccgctgaagaacgtctgctgcacgctatctttggtgagaaggctcgtgaagtccgcgatacctctttgcgcgttccacatggtagtgatggtattatcgttgacgttaaggtgttcacgcgtgagaacggcgatgagctgcctccaggtgtgaatcagctggttcgtgtctatatcgctcagaaacgtaagatttctgagggtgacaagatggccggacgtcacggtaacaagggtgtcgttgcccgtatcctgcctgaagaagatatgccgttccttccggacggtacaccggtacaggttgtcctgaacccgctgggcgtaccttcccgtatgaacatcggacaggtgcttgaagtccatctcggtatggctgcactgcgtctgggtattcacgtggctactccagtatttgacggagcccgtgagtatgacgtgtttgatacgatggaagaagccggtatgcagcgtaacggtaaaactgtgctttatgacggacgtacaggcgaacgctttgagcgtgaagttactgtcggcgtcatgcacatgatcaagctcgcgcacatggttgacgataaaattcatgcccgttctacaggtccttactctctcgttacacagcagccactgggcggtaaggctcagttcggcggacagcgtttcggggaaatggaagtgtgggcgcttgaagcttacggcgcggcatatacactgcaagaaatcttgaccgtgaagtccgatgacgtggtcggccgtgtgaaaacgtacgagtccattgtcaaaggcgaaaatgttccagaaccgggtgttccggaatccttcaaggtattgatcaaggaactgcagtcgctgggtatggatgttaagatccttagcggtgacgagcaggagattgagatgaaggaactggacgatgaggacgagacgtcaggcgataagctgagcctcaatttggaaggcgcagaagtcggcatagagtag

SEQ ID NO: 26 - DNA, 3417 bp, Paenibacillus sonchi
  source 1..3417: mol_type = genomic DNA
ttgcgtgaaatgtttcaggacatctcgccaatccaggatttcacagggaatttggtacttgagttcattgattacagcctaggcgaaccgaagtatacggttgacgacgctaaagagcgggacgtaacatatgcggctcctctacgtgtgaaggtgcggctcattaataaggagaccggtgaggtcaaagagcaggaagtgttcatgggagatttcccgctgatgacggagaccggcacttttattatcaatggtgcggaacgggttattgtcagccagttggttcgctctccaagcgtctatttcagcacgaaagtagataagaacggcaaaaaaacctacaccgccacagtaattccaaaccgcggagcctggcttgaactggagaccgacgctaaggatatcatgtatgtccgtatcgaccggacccgtaaaattccagttacagtgcttctgcgtgctctcggtttcggcagtgatgctgaaattctggaactgctgggtaatgatgaatatattcgcaatacgctggataaagacaatacggactccacggagaaggcgcttattgaaatttacgagcgtctgcgtccgggtgaaccgccgacactggataatgccaagagccttttggttgcacgtttcttcgatccaaagcgttatgacttggccaatgtaggccgttacaaaatcaataagaagctgcacattaagaaccggttgtttaaccagcgtctggcacagcctttggttgatgaatccactggggaaattctggcggaatccggacaaatggttgaccgcagacttcttgatgagctgattccttattttgagaaggatgtcgctgccaagacctaccgtgttaccggcggagtgctggacagtgaagatatcccgctgcaaacgatcgatgtattctcgccgatcgaggaaggccgggttatcaagctgattgccaacggcaatatcgacaaatccgttaagcatattacccaggctgatattatatcctcaatcagctactttattaatctgctgcacggtatcggcaacacagacgacattgaccacttgggtaaccgccgtctgcgttccgtaggtgaattgctgcagaaccaattccgcatcgggctgtcccgtatggaacgcgtagtgcgtgagagaatgtcgattcaggatgcgaatgcaattactccgcaggccctgatcaatattcgtccggtcatcgcgtccattaaagagttcttcggcagctcccagctgtcgcagtttatggaccagacgaacccgctggcagaacttacgcataagcgtcgtctgtctgcactcggacccggcggtctgacccgtgaacgcgcgggctttgaagttcgggacgtgcatcacagtcactacggccggatgtgtccaatcgaaacaccggaaggtccgaatatcggtctgatcaactccttgtccacttttgcccgcatcaatgaatacggctttatcgaagctccgtaccgttgggtggatccgaagaccggcaaggtcacagagcatatcgattatctgaccgccgatgaagaagacaactatgtagttgcgcaggcaaacgtgcagattgatgaagatggaacctttaaggaagatatggttatcgtccgttacaacaaggattcagacaacatcacgactatgcctagtaaccgtgttgactacatggacgtttcgccaaaacaggttgtatcggtcgctacggcgctcattccgttcctggagaacgatgactccaaccgcgcactgatgggatctaacatgcagcgccaagccgttccgcttctgattccgaaggctccgcttgtcggtacaggaatggagcataagtccgctaaagactccggcgtatgtattgtctccaaatatgacggtattattgaacgctcttctgccaatgagatctggctgcgccgggttgaaatggttgaaggcaaagaagtcaaaggtgatatcgttaaatataaattacacaaatttatgcgttcgaaccaagggacttgcataaatcagcgtccgcttgctaaaagaggcgacattgtgaagaagggtgacattcttgcggatggtccttccaccgaaatgggcgagttggctctgggccgcaacgtagttgttgcctttatgacttgggaaggttacaactacgaggatgcgatcctgctgagtgagaagctggtgaaggaagatgtatacacttcgattcatatcgaggaatacgaatccgaagctcgtgacacgaagctgggacctgaagaaattacccgtgatattccaaatgtcggtgaagaagcactccgtaacttggacgaacgcggtatcatccgcattggtgcggaaatcaatgccggtgacatccttgtaggtaaggtaactcctaaaggtgtaactgaactgactgctgaagaacgtctgctccatgcaatcttcggggaaaaagcgcgtgaagttcgcgatacctccttgcgtgttccgcatggtagtgatggtattatcgttgacgtcaaagtattcacacgtgagaacggcgatgagctgcctccgggtgtaaatcagctggttcgtgtctacatcgcccagaaacgtaaaatttctgagggtgacaaaatggccggacgtcacggtaacaagggtgtcgttgcccgtattcttcctgaggaagatatgccattcctgccggacggcacaccggtacaggtagtattgaacccgctgggcgttccttcccgtatgaacatcggacaggtgctggaggtccatcttggtatggccgctcttcgtctgggcattcatgtagctactccggtatttgacggtgcccgcgagtatgacgtctttgatacgatggaagaagccggcatgcagcgcaatggtaagactgtgctgtacgacggacgtacaggcgaacgcttcgagcgtgaagtaacagtcggtgtcatgcacatgatcaagctggcgcacatggttgatgataagattcatgcccgttccacaggtccttactcactcgttacccagcagcctctcggcggtaaagcccagtttggtggacaacgtttcggggaaatggaagtatgggcgcttgaagcttacggcgctgcttatacactgcaggaaatcttgacggtgaaatccgatgacgtggttggccgtgtgaagacgtatgaatccatcgtcaaaggcgagaatctgccggaacctggcgtgcctgaagcgttcaaagtattgatcaaggaactgcagtcgcttggtatggatgtcaaaatcctcagcggtgatgagcaggagattgagatgaaagaactggacgatgaggacgagacgacaggcgacaagctgagccttaacttggaaggcgcggaagtcggaatagagtag

SEQ ID NO: 27 - DNA, 3554 bp, Bacillus megaterium
  source 1..3554: mol_type = genomic DNA
ttactcttttgtgacaggatctttttgtccttcagaaagactttcttcgttatctgcaataattgattctgttggttgctcatggtcctcttctgagtccatgatatcaatttcttgctcgtcagcagaaagcatcttcacatccatacctaaactttgtagttctttaattaatactttgaacgattcaggtatgccaggttctggaatgttttcacctttaacaattgcttcgtatgttttcacacgacctactacgtcatctgatttcactgttaagatctcttgtaatgtgtaagcagcaccgtatgcttcaagtgcccatacctccatctcaccaaaacgctgtccaccgaactgcgctttaccaccaagtggttgttgtgtaacaagtgagtatggtccagtagaacgagcgtgaagtttatcgtctaccatgtgagcaagtttgatcatgtacatgattcctactgatacacggttatcgaatggttcacctgttcgaccatcatatagaactgttttagcatcacgagacatgccagcttcttcgattgttgcccaaacatcttcctcacgcgcaccatcaaatactggagacgcaacgtgaatgccaagcttacgagcagccatacctaaatgaagctctagcacctgaccgatgttcatacgagatggtacccctaatgggtttaacatgatgtcaattggcgtaccgtctggtaggtaaggcatatcttcttcaggtaaaatacgtgaaattacacctttgttaccgtgacgaccggccattttgtcaccttcagaaattttacgcttctgaacaatatatacacggactaattggtttacacctggtggtaattcgtcgccatcttcacggttgaagactttaacatcaagaatgattccaccgccgccgtgcggtacacgaagagaagtatcacgaacttcacgcgctttttcaccgaaaatagcgtgtagaagacgttcttcagctgttagttctgttacaccttttggcgttactttaccaactaaaagatctccgtcttttacttctgcaccaatgcggatgattccacgctcatctaagttgcgaagcgcatcttcacctacgtttggaatgtcacgcgtaatttcttcaggcccaagcttcgtatcacgagactcagattcatattcttcaatatgaacagacgtatatacatcgtctttcacaaggcgttcactcatgatgatggcatcctcatagttgtaaccatcccatgtcatgaaaccaaccattacgtttcgtcctaaagcaagctcacctttttccattgaaggaccgtcggctaagatctcaccttttactacttcgtcgccaactgaaacaattggacgctggttgtaacaagtaccttggttagaacgaacaaattttaataatttgtattgatctaagttacctttaactttttgtccgtctacttcttcatagcggcgaacaaaatttgttttgcttctacgcgctctacaacgccaggatatttacaaatcacggctgcaccagagtctttaccagatacgtattccatacctgtacctacgattggtgcttcaggatttaataaaggtactgcttgacgttgcatgttcgcacccattaatgcacggttagagtcatcgttctctaagaatgggatacatgctgtagcggcagatacaacttgttttggtgatacatccatatagtctaaacgatcgcgacggatgaccgtgttttctccacggaaacgtgcaacgacattttcatctaagaatgaaccatcatcacctagacgagcgttcgcttgggctacaacatagttatcttcttcatcagctgttaagtagtcaattcgctctgtcactttacctgtttcaggatcgatacgacggtaaggtgtttcgatgaaaccgaatttgtttacttttgcataagaagatagtgagttgattaacccgatattcggaccctctggtgtttcaatcggacacatacggccatagtgggagtagtgaacgtcacgcacttcgaaaccagcgcgctcacgcgttaccaccaggtcctagagctgaagacgacgtttgtgcgtcaatcgctaatggattcgtttgatccatgaactgtgataattgagaacttccaaagaactctttaatcgccgcaataactgggcgaatattaattaattgttgaggtgtgattgtattcgtgtcttgaattgacattctttcacgaacaacacgttccatacgggataaaccgatacggaattggttttgtaacaattcactacagaacgcagacgacggttacctaagtggtcgatatcatctgtgtctcctacttgatgaagcaagttaaagaagtaactgattgatgctaaaatatcagcaggtgagatatttttaacttcttctgttacatatgcgttacctgatacagtgatgatttttcaccttctggatcatttggcgcataaatcttaatagattgtaatgtaacatcgtcttctactactccaccaaccggactataagtttgaagtttactccacctcaagcatcggaatcaagcgatctagcagacggcggtcaatcattgcgcctttttctgcaataatttcacctgtttcaggatcaactaatgtttcagctagcttttgattaaataagcgatgtttgatatgaagcttttttattaattttatagcgacctacgttcgctaagtcatatcgttttggatcaaagaagcgagatactaatagagacttcgcattctcaactgtcggtggttcacccggacgtaaacgctcatagatttctaatagcgctttttccgttgtttccgtattatctttttcaagcgtattacggatgtattcattatcaccgattaaatcgataatctcttggtcagaaccgaaacctagcgcgcgtaacagcactgttactggcagttacgagtacgatcaatacgcacgtatactacatcttttgcatcagtttcatattctagccatgcaccacggttagggataacagttgctgtatatccttttttaccgtttttatcaagctttccactatagtaaacacttggtgaacgaaccaactgtgatacgataacacgctcagcaccgttaattacaaaggtacctgtttcggtcatcaatgggaaatctcccatgaacacatcttggtcttttacttcacctgtttctttattaattaaccgcaccttaacacgaagaggtgctgcataagttacatcacgttctttagactctcctactgaatactttggctcacctaagctgtaatcaataaattctagtgataagttacctgtaaaatcatcaattggagaaatatcttggaacatttctcgtaaaccttcatctaaaaaccattggtatgaagccgtttgaatctcgattaagttcggtaactctaaaacttcactaatacgagcataacttctgcgttggcggtggcgtccatactgaactagttgacctgtcaa

SEQ ID NO: 28 - DNA, 867 bp, Paenibacillus borealis
  source 1..867: mol_type = genomic DNA
atgagacaaatagctttctacggtaaaggcggtatcggcaaatcgacaacttcacaaaacaccctggctcagctggcgacaaagttcggacaaagaataatgatcgtaggctgtgaccctaaagcagactccacccgccttatcctgaatacaaaagcccagaactctgtgcttgaactggcggctgagcttggctcggtagaggatcttgaacttgaagatgtattgcagacaggtttcggcgacattatcaacgtagaatgcggcggacctgaaccgggtgtaggctgcgcgggacgcgggatcatcactgccatcaacttcctggagcaggaaggcgcctatcaggatctggatttcgtatcctatgacgttcttggtgacgttgtatgcggcggtttcgcaatgccaatccgcgaaggcaaggcgcaagagatctatatcgtctgttccggtgaaatgatggcaatgtacgcagcgaacaatatcgcccgcgggatcctgaaatatgcgaccagcggcggcgtgagactgggcggcctgatctgcaacagccgtaacacagaccgtgaagatgagctgatcatggaactcgccagacgtctgaacacgcagatgatccactttgtaccgcgtgacaatatcgttcagcatgccgagctgcgcagaatgactgttgcccagtataatccgacccattcacaagcgaaagaatatgaaaagctggctgagaaaatcctcaataacaaaatgctgacgatccctactccgatttctatggaagagctggaagagctgctgatggaattcggcatcatcgaagacgaagaggctgcaatcaagaagctgcaggcttccggtcaataa

SEQ ID NO: 29 - DNA, 870 bp, Paenibacillus sonchi
  source 1..870: mol_type = genomic DNA
atgatgagacaaatagctttctacggtaaaggcggtatcggtaaatccacaacctcccaaaacactttggcccagctcgcaaccaaattcaaacaaagaattatgatcgtaggctgtgacccgaaggcagactccacccgcctgattctgaataccaaggcacagaactcggtcctggagctggcagccgaactgggctcagtagaggatttggaactggaggatgtgctgcagaccggctttggcgacattatcaacgtagagtgcggcggacctgaaccgggtgtaggctgtgcagggcgcggtatcattactgccatcaacttcctggagcaggaaggcgcctatcaggatctggacttcgtatcctatgacgtattgggcgacgttgtatgcggcggtttcgccatgccgatccgtgaaggcaaagcccaagagatctatattgtatgttccggtgaaatgatggcgatgtacgcggccaacaacattgcacgcggtatcctgaaatatgctaccagcggcggcgtgagactgggcggactgatctgcaacagccgcaacaccgaccgtgaagacgagctgatcatggagctggcccgccgtctgaacacgcaaatgatccacttcgttccccgtgacaatatcgttcagcatgccgagctgcgcagaatgacggtggcccaatataaccctgcccatcaacaagccaaagaatatgaaattctggctgaaaaaatcctcaacaacaaaatgctgaccatccctaccccgatttcaatggaagaactggaagagctgctgatggaattcggcatcattgaagatgaagaagctgcactcaagaagctgcaggcttccggccaataa

(19) United States
(12) Patent Application Publication
(10) Pub. No.: US 2026/0250625 A1
(43) Pub. Date: Aug. 27, 2026

(54) METHODS AND SYSTEMS FOR PROMOTING PLANT GROWTH

(71) Applicant: Tenfold Technologies, LLC, Pilot Point, TX (US)

(72) Inventors: Luis Francisco VELAZQUEZ, Grand Prairie, TX (US); Robert D CHISHOLM, McKinney, TX (US); Neissa Maryann PINZON, Frisco, TX (US); Leslie Michelle PERRY, Fort Worth, TX (US); Mohammad Kamrul HASSAN, Aubrey, TX (US); Wendy Loretta STEWARD, Pilot Point, TX (US); Maud Ann WRIGHTSON HINCHEE, Little Elm, TX (US); Allana Kay WELSH, Denton, TX (US); Shashi Shankar RAJBANSHI, Frisco, TX (US)

(21) Appl. No.: 19/426,299

(22) Filed: Dec. 19, 2025

Related U.S. Application Data
(63) Continuation of application No. PCT/US2024/034792, filed on Jun. 20, 2024.
Application No. PCT/US2024/034792 is a continuation of application No. 18/541,671, filed on Dec. 15, 2023, now Pat. No. 12,365,868.
Application No. 18/541,671 is a continuation of application No. 18/541,917, filed on Dec. 15, 2023, now Pat. No. 12,365,869.
(60) Provisional application No. 63/509,263, filed on Jun. 20, 2023.
(60) Provisional application No. 63/510,615, filed on Jun. 27, 2023.
(60) Provisional application No. 63/610,535, filed on Dec. 15, 2023.
(60) Provisional application No. 63/613,548, filed on Dec. 21, 2023.
(60) Provisional application No. 63/613,560, filed on Dec. 21, 2023.

(51) Int. Cl. C12N 1/20; A01N 63/22; A01N 63/25; A01P 21/00; C05F 11/08; C12M 1/00; C12M 1/02

(52) U.S. Cl. CPC C12N 1/20; A01N 63/22; A01N 63/25; A01P 21/00; C05F 11/08; C12M 23/58; C12M 27/00; C12M 29/18; C12M 47/02

(57) ABSTRACT

The present disclosure provides methods and systems for production of biostimulants that promote growth in plants. Embodiments described include methods of making a biostimulant composition in a bioreactor system that includes two or more containers arranged in series. The bioreactor system may include an established population of a microbial strain. The method may include operating the bioreactor system by transferring into the system an aqueous feedstock that comprises a microbial consortium, transferring working fluid between containers of the system, and collecting a product. The method may further include maintaining a concentration of a microbial strain in the system.

### CROSS-REFERENCE

This application is a continuation of International Patent Application No. PCT/US2024/034792, filed Jun. 20, 2024, which claims the benefit of U.S. patent application Ser. No. 18/541,671, filed Dec. 15, 2023, now U.S. Pat. No. 12,365,868, issued Jul. 22, 2025, U.S. patent application Ser. No. 18/541,917, filed Dec. 15, 2023, now U.S. Pat. No. 12,365,869, issued Jul. 22, 2025, U.S. Provisional Application No. 63/509,263, filed Jun. 20, 2023, U.S. Provisional Application No. 63/510,615, filed Jun. 27, 2023, and U.S. Provisional Application No. 63/610,535, filed Dec. 15, 2023, each of which is entirely incorporated herein by reference. This application claims the benefit of U.S. Provisional Application No. 63/613,548, filed Dec. 21, 2023, the content of which is entirely incorporated herein by reference. This application claims the benefit of U.S. Provisional Application No. 63/613,560, filed Dec. 21, 2023, the content of which is entirely incorporated herein by reference.

### SEQUENCE LISTING

The instant application contains a Sequence Listing which has been submitted electronically in XML file format and is hereby incorporated by reference in its entirety. Said XML copy, created on Apr. 22, 2026, is named 63472-720_301_SL.xml and is 90,136 bytes in size.

### BACKGROUND

The disclosure is generally related to biostimulant compositions and methods of using such biostimulant compositions to promote plant growth.

Promoting efficient production of food crops and other crops is an important goal for environmental and economic reasons. Plant growth promoting products sourced from organic materials can help to enhance crop growth, improve the efficacy of agricultural products, such as fertilizers, and reduce the environmental impacts of synthetic fertilizers and climate change. There exists a need for plant growth promoting biostimulant compositions that use abundant and available organic feedstocks.

### SUMMARY

In an aspect, the present disclosure provides a method of making a biostimulant composition having a plant growth promoting property, the method comprising: (a) providing a bioreactor system comprising two or more containers arranged in a series, each of the two or more containers comprising a volume of a working fluid, wherein a first container comprises an established population of a microbial strain having the plant growth promoting property; (b) operating the bioreactor system for a duration of time by: (i) transferring into the first container an aqueous feedstock comprising a microbial consortium; (ii) transferring a portion of the working fluid out of each of the two or more containers into either a subsequent container of the bioreactor system or a product outflow stream; (iii) maintaining a concentration of the microbial strain throughout the duration of time in at least the first container at at least 80% of a concentration of the microbial strain at the beginning of the duration of time; and (iv) collecting at least a portion of the product outflow stream as the biostimulant composition, wherein the biostimulant composition has the plant growth promoting property; wherein the duration of time is at least 5 days; and wherein the microbial strain is not present in the aqueous feedstock or any other input into the bioreactor system during the duration of time at a concentration that is higher than 1% of the concentration of the microbial strain in the first container.

In some embodiments, the maintaining of step (b) (iii) comprises maintaining the concentration of the microbial strain at at least 1×10³ CFU/ml. In some embodiments, the maintaining of step (b) (iii) comprises providing nutrients in the first container that selectively promote growth of the microbial strain and sustain the established population. In some embodiments, the maintaining of step (b) (iii) comprises applying selective pressure in at least the first container. In some embodiments, the selective pressure comprises conditions that reduce the growth rate of microbes present in the first container relative to the growth rate of the microbial strain in the first container. In some embodiments, the selective pressure comprises conditions that shifts an ionic concentration of the working fluid. In some embodiments, the microbial strain, microbes of the microbial consortium, or any combination thereof may be enriched following the shift in the ionic concentration of the working fluid. In some embodiments, the selective pressure increases an activity of microbes within the microbial consortium. In some embodiments, the activity comprises solubilization of an inorganic substrate. In some embodiments, the inorganic substrate comprises phosphate or zinc.

In some embodiments, the biostimulant composition comprises an amount of the microbial strain. In some embodiments, the amount of the microbial strain is effective to impart the plant growth promoting property to the biostimulant composition. In some embodiments, the amount of the microbial strain is 1×10² to 1×10⁶ CFU/ml.

In some embodiments, the aqueous feedstock further comprises an organic substrate. In some embodiments, the organic substrate comprises manure, lignocellulosic material, wastewater biosolids, food waste, energy crops, yeast, guano, agricultural waste, algae, or any combination thereof. In some embodiments, the biostimulant composition comprises digestion products produced by digestion of the organic substrate by the microbial strain and/or the microbial consortium. In some embodiments, the biostimulant composition comprises metabolites produced by the microbial strain and/or the microbial consortium. In some embodiments, the metabolites and/or digestion products in the biostimulant composition are present in an amount effective to impart the plant growth promoting property to the biostimulant composition. In some embodiments, the biostimulant composition retains the plant growth promoting property after filter sterilization. In some embodiments, the plant growth promoting property comprises an ability to promote uptake of a nutrient by a plant, increase the bioavailability of a nutrient in soil, recruit microbes having the plant growth promoting property to the root zones or other tissues of plants, stimulate plant growth, increase yield of a crop, increase shoot biomass, increase root biomass, increase deaminase activity, increase acid production, increase leaf area, increase chlorophyll content, increase heat tolerance, increase cold tolerance, increase drought tolerance, increase salt tolerance, increase total biomass, or any combination thereof. In some embodiments, the plant growth promoting property comprises an ability to increase nitrogen use efficiency, zinc solubilization, or phosphate solubilization.

In another aspect, the present disclosure provides a method of making a biostimulant composition having a desired plant growth promoting property, the method comprising: (a) providing a bioreactor system comprising two or more containers arranged in a series, each of the two or more containers comprising a volume of a working fluid, wherein a first container comprises an established population of a microbial strain having the desired plant growth promoting property; (b) operating the bioreactor system for a duration of time by: (i) transferring into the first container an aqueous feedstock comprising a microbial consortium; (ii) transferring a portion of the working fluid out of each of the two or more containers into either a subsequent container of the bioreactor system or a product outflow stream; and (iii) collecting at least a portion of the product outflow stream as the biostimulant composition; wherein the established population of the microbial strain is not present in an aqueous feedstock or any other input in the bioreactor system during the duration of time at a concentration that is higher than 1% of the concentration of the established population of the microbial strain in the first container.

In another aspect, the present disclosure provides a method, comprising: (a) providing a bioreactor system comprising two or more containers arranged in a series, each of the two or more containers comprising a volume of a working fluid, wherein a first container comprises an established population of a microbial strain having a plant growth promoting property; (b) operating the bioreactor system for a duration of time by: (i) transferring into the first container an aqueous feedstock comprising a microbial consortium; (ii) transferring a portion of the working fluid out of each of the two or more containers into either a subsequent container of the bioreactor system or a product outflow stream; and (iii) collecting at least a portion of the product outflow stream as a biostimulant composition, wherein the biostimulant composition comprises an amount of the microbial strain; wherein the amount of the microbial strain is configured to increase nutrient uptake of a plant administered the biostimulant composition as compared to a nutrient uptake of a plant not administered the biostimulant composition.

In some embodiments, the nutrient uptake is measured by one or more of analysis of biomass, depletion method, or nutrient balance method, or by an assay as described in Example 4. In some embodiments, the nutrient uptake comprises macronutrients comprising nitrogen, sulfur, phosphorous, potassium, magnesium, calcium, or a combination thereof. In some embodiments, the nutrient uptake comprises micronutrients comprising boron, zinc, manganese, iron, copper, or a combination thereof.

In another aspect, the present disclosure provides a method, comprising: (a) providing a bioreactor system comprising two or more containers arranged in a series, each of the two or more containers comprising a volume of a working fluid, wherein a first container comprises an established population of a microbial strain having a plant growth promoting property; (b) operating the bioreactor system for a duration of time by: (i) transferring into the first container an aqueous feedstock comprising a microbial consortium; (ii) transferring a portion of the working fluid out of each of the two or more containers into either a subsequent container of the bioreactor system or a product outflow stream; and (iii) collecting at least a portion of the product outflow stream as a biostimulant composition, wherein the biostimulant composition comprises an amount of the microbial strain; wherein the biostimulant composition is configured to promote a plant growth property of a plant administered the biostimulant composition as compared to that of a plant not administered the biostimulant composition.

In some embodiments, the biostimulant composition is configured to promote uptake of a nutrient by a plant, increase the bioavailability of a nutrient in soil, recruit microbes having the plant growth promoting property to the root zones or other tissues of plants, stimulate plant growth, increase yield of a crop, increase shoot biomass, increase root biomass, increase deaminase activity, increase acid production, increase leaf area, increase chlorophyll content, increase heat tolerance, increase cold tolerance, increase drought tolerance, increase salt tolerance, increase total biomass, or any combination thereof. In some embodiments, the total biomass of the plant administered the biostimulant composition is increased by at least 5% as compared to the plant not administered the biostimulant composition. In some embodiments, the total biomass of the plant administered the biostimulant composition is increased by at least 10% as compared to the plant not administered the biostimulant composition. In some embodiments, an average leaf area of the plant administered the biostimulant composition is increased by at least 10% compared the plant not administered the biostimulant composition. In some embodiments, an average leaf area of the plant administered the biostimulant composition is increased by at least 25% compared the plant not administered the biostimulant composition.

In some embodiments, the established population of the microbial strain comprises a zinc solubilizing microbial strain. In some embodiments, administering the biostimulant composition to the plant increases an average leaf area by at least 25% as compared to an average leaf area of a plant not administered the biostimulant composition. In some embodiments, administering the biostimulant composition to the plant increases an average leaf area by at least 10% compared to an average leaf area of a plant not administered the biostimulant composition. In some embodiments, administering the biostimulant composition to a plant increases a nitrogen content, a phosphorous content, a potassium content, or any combination thereof by at least 20 ug compared to that of a plant not administered the biostimulant composition.

In some embodiments, the established population of the microbial strain comprises a phosphate solubilizing microbial strain. In some embodiments, administering the biostimulant composition to one or more plants increases a crop yield by at least 5% as compared to a crop yield of one or more plants not administered the biostimulant composition. In some embodiments, administering the biostimulant composition to one or more plants increases a dry biomass by at least 25% compared a dry biomass of one or more plants not administered the biostimulant composition.

In some embodiments, administering the biostimulant composition to a plant increases an average leaf area by at least 10% compared to an average leaf area of a plant not administered the biostimulant composition. In some embodiments, the established population of the microbial strain comprises a nitrogen use efficiency-promoting microbial strain. In some embodiments, administering the biostimulant composition to the plant increases an average leaf area by at least 50% compared to an average leaf area of a plant not administered the biostimulant composition. In some embodiments, administering the biostimulant composition to the plant increases a dry shoot weight, a dry root weight, a stem diameter, or any combination thereof by at least 10% compared to that of a plant not administered the biostimulant composition. In some embodiments, administering the biostimulant composition to the plant increases a leaf chlorophyll content by at least 10% compared to that of a plant not administered the biostimulant composition.

In some embodiments, the established population of the strain in (b) is incubated for a duration of time. In some embodiments, the duration of time is at least 5 days.

In some embodiments, the method further comprises in (b), operating the bioreactor system for the duration of time by: (iii) maintaining a concentration of the microbial strain throughout the duration of time in at least the first container at at least 80% of a concentration of the microbial strain at the beginning of the duration of time. In some embodiments, the maintaining of step (b) (iii) comprises maintaining the concentration of the microbial strain at at least 1×10³ CFU/ml. In some embodiments, the maintaining of step (b) (iii) comprises providing nutrients in the first container that selectively promote growth of the microbial strain and sustain the established population. In some embodiments, the maintaining of step (b) (iii) comprises applying selective pressure in at least the first container.

In some embodiments, the selective pressure comprises conditions that reduce the growth rate of microbes present in the first container relative to the growth rate of the microbial strain in the first container. In some embodiments, the selective pressure comprises conditions that shifts an ionic concentration of the working fluid. In some embodiments, the microbial strain, microbes of the microbial consortium, or any combination thereof may be enriched following the shift in the ionic concentration of the working fluid. In some embodiments, the desired plant growth promoting property comprises an ability to promote uptake of a nutrient by one or more plants, increase bioavailability of a nutrient in soil, recruit microbes having the desired plant growth promoting property to root zones or other tissues of plants, stimulate plant growth, increase yield of a crop, increase shoot biomass, increase root biomass, increase deaminase activity, increase acid production, increase leaf area, increase chlorophyll content, increase heat tolerance, increase cold tolerance, increase drought tolerance, increase salt tolerance, increase total biomass, or any combination thereof. In some embodiments, the nutrient comprises macronutrients. In some embodiments, the macronutrients comprises nitrogen, sulfur, phosphorous, potassium, magnesium, calcium, or a combination thereof. In some embodiments, the nutrient comprises micronutrients.

In some embodiments, the micronutrients comprises boron, zinc, manganese, iron, copper, or any combination thereof.

In some embodiments, the microbial strain is not present in the aqueous feedstock or any other input into the bioreactor system during the duration of time. In some embodiments, the microbial strain is present in the aqueous feedstock or any other input into the bioreactor system during the duration of time. In some embodiments, the method further comprises, before (a), adding an inoculum of the microbial strain to the bioreactor system, wherein the inoculum of the microbial strain produces an initial population of the microbial strain of at least 0.5×10⁴ CFU/ml in at least one container. In some embodiments, before adding the inoculum of the microbial strain, the concentration of the microbial strain is less than 1×10² CFU/ml. In some embodiments, the aqueous feedstock further comprises an organic material at least partially digestible by microbes present in at least one of the containers. In some embodiments, before the transferring of step (b) (i), the organic material had been partially digested by microbes endogenous to the organic material. In some embodiments, the method further comprises digesting the organic material in two or more serially connected containers before the transferring of step (b) (i).

In some embodiments, the organic material comprises manure and/or material produced by microbial digestion of manure. In some embodiments, the aqueous feedstock further comprises an inorganic material. In some embodiments, the inorganic material comprises rock phosphate particles. In some embodiments, prior to the transferring of step (b) (i), the rock phosphate particles had been partially digested by microbes present in the aqueous feedstock. In some embodiments, the method further comprises partially digesting the rock phosphate particles in two or more serially connected containers before the transferring of step (b) (i). In some embodiments, the microbial consortium comprises at least 1×10⁵ CFU/ml. In some embodiments, the microbial consortium comprises microbes derived from manure and from rock phosphate particles.

In some embodiments, the operating of step (b) further comprises producing microbial metabolites that have the plant growth promoting property. In some embodiments, the transferring of step (b) (i), the transferring of step (b) (ii), and the collecting of step (b) are performed continuously throughout the duration of time. In some embodiments, the transferring of step (b), the transferring of step (b), and the collecting of step (b) are performed periodically throughout the duration of time. In some embodiments, the method further comprises adding one or more carbon sources to at least one container of the bioreactor system.

In some embodiments, the one or more carbon sources are comprised in the aqueous feedstock. In some embodiments, the bioreactor system comprises a clarifier container comprising a clarifier working fluid. In some embodiments, the method further comprises further comprising separating a supernatant portion of the clarifier working fluid from a floc portion of the clarifier working fluid within the clarifier container. In some embodiments, the separating comprises gravity separation.

In some embodiments, the method further comprises folding the floc portion of the clarifier working fluid. In some embodiments, the folding further comprises releasing a population of the microbial strain into the supernatant portion without introducing floc solids into the supernatant portion. In some embodiments, the folding is performed by folding wipers in a bottom portion of the clarifier container. In some embodiments, the operating further comprises transferring the floc portion from the clarifier container to an earlier container in the bioreactor system. In some embodiments, the product outflow stream comprises the supernatant portion of the clarifier working fluid. In some embodiments, the method further comprises producing at least 1×10⁴ CFU/ml of the microbial strain in the product outflow stream.

In some embodiments, the bioreactor system comprises the first container comprising a volume of a first working fluid, a second container comprising a volume of a second working fluid, and a third container comprising a volume of a third working fluid. In some embodiments, at least one of the first working fluid, the second working fluid, or the third working fluid comprises a pH buffering system. In some embodiments, the method further comprises maintaining the pH of at least one of the first working fluid, the second working fluid, or the third working fluid between 6 and 9 throughout the duration of time. In some embodiments, the first container comprises an outlet port fluidly connected to an inlet port of the second container and the second container comprises an outlet port fluidly connected to an input port of the third container.

In some embodiments, the third container comprises an outlet port fluidly connected to a clarifier container. In some embodiments, the method further comprises maintaining the volume of each of the first working fluid, the second working fluid, and the third working fluid constant throughout the duration of time. In some embodiments, step (b) comprises operating the bioreactor system in a hydraulically balanced manner. In some embodiments, the transferring of step (b), the transferring of step (b, and the collecting of step (b) are driven by gravity.

In some embodiments, the operating comprises maintaining a flow rate that results in a hydraulic retention time of at least 5 days. In some embodiments, the operating comprises maintaining the product outflow stream at a flow rate of at least 100 gallons per day. In some embodiments, the volume of working fluid in each of the two or more containers is at least 100 gallons. In some embodiments, at least one of the two or more containers is a fluidized bed reactor. In some embodiments, at least one of the two or more containers is a packed bed reactor. In some embodiments, the method further comprises maintaining at least one of the two or more containers under aerobic conditions. In some embodiments, the method further comprises maintaining at least one of the two or more containers under microaerobic conditions.

In some embodiments, the bioreactor system is operated continuously for at least 90 days. In some embodiments, the aqueous feedstock does not comprise the microbial strain at a concentration higher than 10 CFU/ml. In some embodiments, the microbial strain is not added to the bioreactor system during the duration of time at a concentration that is higher than 10 CFU/ml. In some embodiments, the bioreactor system comprises at least one container placed in the series before the first container. In some embodiments, the volume of working fluid of one of the two or more containers comprises the microbial consortium, wherein each microbial consortium is distinct from all of the microbial consortia in other working fluids. In some embodiments, the method further comprises producing a population of sporulated bacteria in the product outflow stream. In some embodiments, the method further comprises producing a population of the microbial strain in the product outflow stream that is sporulated. In some embodiments, the population of the microbial strain that is sporulated comprises at least 1×10² CFU/ml. In some embodiments, the method further comprises adding an additional population of the microbial strain to the biostimulant product.

In some embodiments, the plant growth promoting property does not comprise nitrogen use efficiency, zinc solubilization, or phosphate solubilization. In some embodiments, the microbial strain is a nitrogen use efficiency-promoting microbial strain. In some embodiments, the nitrogen use efficiency-promoting microbial strain is one that performs nitrogen fixation, promotes nitrogen fixation in the tissues of plants, recruits nitrogen fixers to the root zones or other tissues of plants, or increases organic nitrogen content and/or mineralization of organic nitrogen in soil. In some embodiments, the nitrogen use efficiency-promoting microbial strain is positive for a nifH gene. In some embodiments, the nitrogen use efficiency-promoting microbial strain is one that promotes plant growth in a nitrogen-poor growth medium. In some embodiments, the nitrogen-poor growth medium comprises nitrate at less than 10 ppm.

In some embodiments, the nitrogen use efficiency-promoting microbial strain is of the genus Kosakonia, Klebsiella, Rahnella, Kluyvera, Enterobacter, Achromobacter, Microbacterium, Gluconobacter, Methylobacterium, Pseudomonas, Pantoea, Azospirillum, Azocarus, Herbaspirillum, Burkholderia, Cyanobacteria, Bacillus, Paenibacillus, or any combination thereof. In some embodiments, the nitrogen use efficiency-promoting microbial strain is of the species Kosakonia sacchari, Klebsiella variicola, Rahnella aquatilis, Kluyvera intermedia, Kosakonia pseusosacchari, Enterobacter spp., Achromobacter marplatensis, Azopirillum lipoferum, Microbacterium murale, Gluconobacter diazotrophicus, Methylobacterium symbioticum, Paenibacillus borealis, Bacillus megaterium (Priestia megaterium), or Paenibacillus sonchi. In some embodiments, the nitrogen use efficiency-promoting microbial strain is the strain deposited under ATCC Accession No. PTA-127654 (MS3907), the strain deposited under ATCC Accession No. PTA-127653 (MS3900), the strain deposited under ATCC Accession No. PTA-127655 (MS4921), or the strain deposited under ATCC Accession No. PTA-127652 (MS2748). In some embodiments, the nitrogen use efficiency-promoting microbial strain is not present in the aqueous feedstock or any other input into the bioreactor system during the duration of time at a concentration of greater than 100 CFU/ml.

In some embodiments, the microbial strain is a zinc solubilizing bacterial strain. In some embodiments, the zinc-solubilizing bacterial strain is of the genus Bacillus. In some embodiments, the zinc-solubilizing bacterial strain is of the species Bacillus safensis or Bacillus megaterium.

In some embodiments, the zinc-solubilizing bacterial strain is one of the following: (a) a Bacillus safensis strain having one or more of the following: (i) a 16S rRNA gene sequence at least 95% identical to SEQ ID NO: 1; (ii) a gyrB gene sequence at least 95% identical to SEQ ID NO: 4; and (iii) a rpoB gene sequence at least 95% identical to SEQ ID NO: 7; (b) a Bacillus megaterium strain having one or more of the following: (i) a 16S rRNA gene sequence at least 95% identical to SEQ ID NO: 2; (ii) a gyrB gene sequence at least 95% identical to SEQ ID NO: 5; and (iii) a rpoB gene sequence at least 95% identical to SEQ ID NO: 8; or (c) a Bacillus megaterium strain having one or more of the following: (i) a 16S rRNA gene sequence at least 95% identical to SEQ ID NO: 3; (ii) a gyrB gene sequence at least 95% identical to SEQ ID NO: 6; and (iii) a rpoB gene sequence at least 95% identical to SEQ ID NO: 9.

In some embodiments, the zinc-solubilizing bacterial strain is the Bacillus safensis strain deposited under ATCC Accession No. PTA-127681, the Bacillus megaterium strain deposited under ATCC Accession No. PTA-127683, or the Bacillus megaterium strain deposited under ATCC Accession No. PTA-127682. In some embodiments, the microbial strain is a phosphate solubilizing bacterial strain. In some embodiments, the phosphate solubilizing bacterial strain is of the genus Bacillus. In some embodiments, the phosphate solubilizing bacterial strain is of the species Bacillus amyloliquefaciens or Bacillus licheniformis. In some embodiments, the phosphate solubilizing bacterial strain is one of the following: (a) a Bacillus amyloliquefaciens strain having one or more of the following: (i) a 16S rRNA gene sequence at least 95% identical to SEQ ID NO: 10; (ii) a gyrB gene sequence at least 95% identical to SEQ ID NO: 12; and (iii) a rpoB gene sequence at least 95% identical to SEQ ID NO: 14; or (b) a Bacillus licheniformis strain having one or more of the following: (i) a 16S rRNA gene sequence at least 95% identical to SEQ ID NO: 11; (ii) a gyrB gene sequence at least 95% identical to SEQ ID NO: 13; and (iii) a rpoB gene sequence at least 95% identical to SEQ ID NO: 15.

In some embodiments, the phosphate solubilizing bacterial strain is the Bacillus amyloliquefaciens strain deposited under ATCC Accession No. PTA-127657 or the Bacillus licheniformis strain deposited under ATCC Accession No. PTA-127656. In some embodiments, the phosphate solubilizing bacterial strain is not present in the aqueous feedstock or any other input into the bioreactor system during the duration of time at a concentration of greater than 100 CFU/ml.

In an aspect, the present disclosure provides a biostimulant composition made by the method described herein.

In another aspect, the present disclosure provides a method of promoting plant growth comprising contacting a plant, seed, or plant growth medium with the biostimulant composition described herein.

In an aspect, the present disclosure provides a bioreactor system comprising: (i) a stream of an aqueous feedstock in fluid communication with a first container comprising a volume of a first working fluid, wherein the aqueous feedstock comprises a microbial consortium, wherein the first working fluid comprises a population of a microbial strain having a desired plant growth promoting property, wherein a concentration of the microbial strain in the first working fluid is at least 100 times higher than a concentration of the microbial strain in the aqueous feedstock stream or in any other input into the bioreactor system; (ii) one or more additional containers arranged in a series that comprises the first container, wherein each of the one or more additional containers comprises a volume of a working fluid and is in fluid communication with at least one other container in the series, and wherein at least one of the one or more additional containers comprises a product outflow stream port; and (iii) a product outflow stream in fluid communication with the product outflow stream port.

In another aspect, the present disclosure provides a bioreactor system comprising: (i) a stream of an aqueous feedstock in fluid communication with a first container comprising a volume of a first working fluid, wherein the aqueous feedstock comprises a microbial consortium, wherein the first working fluid comprises a population of a microbial strain having a desired plant growth promoting property; (ii) one or more additional containers arranged in a series that comprises the first container, wherein each of the one or more additional containers comprises a volume of a working fluid and is in fluid communication with at least one other container in the series, and wherein at least one of the one or more additional containers comprises a product outflow stream port; and (iii) a product outflow stream in fluid communication with the product outflow stream port, wherein the product outflow stream comprises a biostimulant composition, wherein the biostimulant composition comprises an amount of the microbial strain, wherein the amount of the microbial strain is configured to increase nutrient uptake of a plant administered the biostimulant composition as compared to a nutrient uptake of the plant not administered the biostimulant composition.

In another aspect, the present disclosure provides a bioreactor system comprising: (i) a stream of an aqueous feedstock in fluid communication with a first container comprising a volume of a first working fluid, wherein the aqueous feedstock comprises a microbial consortium, wherein the first working fluid comprises a population of a microbial strain having a desired plant growth promoting property; (ii) one or more additional containers arranged in a series that comprises the first container, wherein each of the one or more additional containers comprises a volume of a working fluid and is in fluid communication with at least one other container in the series, and wherein at least one of the one or more additional containers comprises a product outflow stream port; and (iii) a product outflow stream in fluid communication with the product outflow stream port, wherein the product outflow stream comprises a biostimulant composition, wherein the biostimulant composition comprises an amount of the microbial strain, wherein the amount of the microbial strain is configured to promote a plant growth promoting property of a plant administered the biostimulant composition as compared to the plant not administered the biostimulant composition.

In some embodiments, the desired plant growth promoting property comprises an ability to promote uptake of a nutrient by one or more plants, increase bioavailability of a nutrient in soil, recruit microbes having the desired plant growth promoting property to root zones or other tissues of plants, stimulate plant growth, increase yield of a crop, increase shoot biomass, increase root biomass, increase deaminase activity, increase acid production, increase leaf area, increase chlorophyll content, increase heat tolerance, increase cold tolerance, increase drought tolerance, increase salt tolerance, increase total biomass, or any combination thereof. In some embodiments, the nutrient comprises macronutrients. In some embodiments, the macronutrients comprises nitrogen, sulfur, phosphorous, potassium, magnesium, calcium, or a combination thereof. In some embodiments, the nutrient comprises micronutrients. In some embodiments, the micronutrients comprises boron, zinc, manganese, iron, copper, or any combination thereof.

In some embodiments, the bioreactor system is a continuous flow bioreactor system and the stream of the aqueous feedstock is a continuous stream. In some embodiments, each of the volume of the working fluid is constant. In some embodiments, each of the first container and the one or more additional containers comprises a concentration of the microbial strain that remains at least 1×10⁴ CFU/ml during operation of the bioreactor system. In some embodiments, the aqueous feedstock and any other input into the bioreactor system does not comprise the population of the microbial strain or does not comprise a concentration of the microbial strain at level higher than 100 CFU/ml. In some embodiments, the microbial consortium comprises at least 1×10⁴ CFU/ml. In some embodiments, the aqueous feedstock further comprises an organic material digestible by microbes present in the container.

In some embodiments, the organic material comprises manure or material derived from manure. In some embodiments, the aqueous feedstock further comprises rock phosphate particles. In some embodiments, the microbial consortium comprises microbes derived from manure and rock phosphate particles. In some embodiments, the container comprising the product outflow stream port is a clarifier container configured to separate a portion of a working fluid in the clarifier container into a supernatant portion and a floc portion. In some embodiments, the clarifier container comprises one or more floc folding flights configured to agitate settled floc in the clarifier container without resuspending solids in the floc portion into the supernatant portion. In some embodiments, the system further comprises a floc return stream that flows from the clarifier to an earlier container in the series.

In some embodiments, the product outflow stream comprises the supernatant portion. In some embodiments, the product outflow stream comprises at least 1×10⁴ CFU/ml of the microbial strain. In some embodiments, the product outflow stream comprises at least 1×10² CFU/ml of a sporulated form of the microbial strain. In some embodiments, the product outflow stream comprises a total dry weight of 0.2 to 2.5 mg/ml. In some embodiments, the product outflow stream has a chemical oxygen demand between 80 to 500 mg/L. In some embodiments, the product outflow stream has an electrical conductivity between 0.1 and 3.0 mS/cm. In some embodiments, at least the first container comprises a mixer configured to aerate the first working fluid. In some embodiments, the first working fluid and/or the working fluid in at least one of the one or more additional containers comprises aerobic conditions. In some embodiments, the microbial strain is a nitrogen use efficiency-promoting microbial strain.

In some embodiments, the nitrogen use efficiency-promoting microbial strain is one that performs nitrogen fixation, promotes nitrogen fixation in the tissues of plants, recruits nitrogen fixers to the root zones or other tissues of plants, or increases organic nitrogen content and/or mineralization of organic nitrogen in soil. In some embodiments, the nitrogen use efficiency-promoting microbial strain is positive for a nifHI gene. In some embodiments, the nitrogen use efficiency-promoting microbial strain is one that promotes plant growth in a nitrogen-poor growth medium. In some embodiments, the nitrogen-poor growth medium comprises nitrate at less than 10 ppm.

In some embodiments, the nitrogen use efficiency-promoting microbial strain is of the genus Kosakonia, Klebsiella, Rahnella, Kluyvera, Enterobacter, Achromobacter, Microbacterium, Gluconobacter, Methylobacterium, Pseudomonas, Pantoea, Azospirillum, Azocarus, Herbaspirillum, Burkholderia, Cyanobacteria, Bacillus, Paenibacillus, or any combination thereof. In some embodiments, the nitrogen use efficiency-promoting microbial strain is of the species Kosakonia sacchari, Klebsiella variicola, Rahnella aquatilis, Kluyvera intermedia, Kosakonia pseusosacchari, Enterobacter spp., Achromobacter marplatensis, Azopirillum lipoferum, Microbacterium murale, Gluconobacter diazotrophicus, Methylobacterium symbioticum, Paenibacillus borealis, Bacillus megaterium (Priestia megaterium), or Paenibacillus sonchi. In some embodiments, the nitrogen use efficiency-promoting microbial strain is the strain deposited under ATCC Accession No. PTA-127654 (MS3907), the strain deposited under ATCC Accession No. PTA-127653 (MS3900), the strain deposited under ATCC Accession No. PTA-127655 (MS4921), or the strain deposited under ATCC Accession No. PTA-127652 (MS2748).

In some embodiments, the microbial strain is a phosphate solubilizing bacterial strain. In some embodiments, the phosphate solubilizing bacterial strain is of the genus Bacillus. In some embodiments, the phosphate solubilizing bacterial strain is of the species Bacillus amyloliquefaciens or Bacillus licheniformis. In some embodiments, the phosphate solubilizing bacterial strain is one of the following: (a) a Bacillus amyloliquefaciens strain having one or more of the following: (i) a 16S rRNA gene sequence at least 95% identical to SEQ ID NO: 10; (ii) (ii) a gyrB gene sequence at least 95% identical to SEQ ID NO: 12; and (iii) (iii) arpoB gene sequence at least 95% identical to SEQ ID NO: 14; or (b) a Bacillus licheniformis strain having one or more of the following: (i) a 16S rRNA gene sequence at least 95% identical to SEQ ID NO: 11; (ii) a gyrB gene sequence at least 95% identical to SEQ ID NO: 13; and (iii) a rpoB gene sequence at least 95% identical to SEQ ID NO: 15. In some embodiments, the phosphate solubilizing bacterial strain is the Bacillus amyloliquefaciens strain deposited under ATCC Accession No. PTA-127657 or the Bacillus licheniformis strain deposited under ATCC Accession No. PTA-127656.

In some embodiments, the microbial strain is a zinc solubilizing bacterial strain. In some embodiments, the zinc-solubilizing bacterial strain is of the genus Bacillus. In some embodiments, the zinc-solubilizing bacterial strain is of the species Bacillus safensis or Bacillus megaterium. In some embodiments, the zinc-solubilizing bacterial strain is one of the following: (a) a Bacillus safensis strain having one or more of the following: (i) a 16S rRNA gene sequence at least 95% identical to SEQ ID NO: 1; (ii) a gyrB gene sequence at least 95% identical to SEQ ID NO: 4; and (iii) a rpoB gene sequence at least 95% identical to SEQ ID NO: 7; (b) a Bacillus megaterium strain having one or more of the following: (i) a 16S rRNA gene sequence at least 95% identical to SEQ ID NO: 2; (ii) a gyrB gene sequence at least 95% identical to SEQ ID NO: 5; and (iii) a rpoB gene sequence at least 95% identical to SEQ ID NO: 8; or (c) a Bacillus megaterium strain having one or more of the following: (i) a 16S rRNA gene sequence at least 95% identical to SEQ ID NO: 3; (ii) a gyrB gene sequence at least 95% identical to SEQ ID NO: 6; and (iii) a rpoB gene sequence at least 95% identical to SEQ ID NO: 9. In some embodiments, the zinc-solubilizing bacterial strain is the Bacillus safensis strain deposited under ATCC Accession No. PTA-127681, the Bacillus megaterium strain deposited under ATCC Accession No. PTA-127683, or the Bacillus megaterium strain deposited under ATCC Accession No. PTA-127682.

In some embodiments, the nitrogen use efficiency-promoting microbial strain is one of the following: (a) a Bacillus megaterium strain having one or more of the following: a 16S rRNA gene sequence at least 95% identical to SEQ ID NO: 16; a gyrB gene sequence at least 95% identical to SEQ ID NO: 20; and a rpoB gene sequence at least 95% identical to SEQ ID NO: 24; (b) a Paenibacillus borealis strain having one or more of the following: a 16S rRNA gene sequence at least 95% identical to SEQ ID NO: 17; a gyrB gene sequence at least 95% identical to SEQ ID NO: 21; and a rpoB gene sequence at least 95% identical to SEQ ID NO: 25; (c) a Paenibacillus sonchi strain having one or more of the following: a 16S rRNA gene sequence at least 95% identical to SEQ ID NO: 18; a gyrB gene sequence at least 95% identical to SEQ ID NO: 22; and a rpoB gene sequence at least 95% identical to SEQ ID NO: 26; or (d) a Bacillus megaterium strain having one or more of the following: a 16S rRNA gene sequence at least 95% identical to SEQ ID NO: 19; a gyrB gene sequence at least 95% identical to SEQ ID NO: 23; and a rpoB gene sequence at least 95% identical to SEQ ID NO: 27.

In an aspect, the present disclosure provides a biostimulant composition made by the system described herein.

In an aspect, the present disclosure provides A method of promoting plant growth comprising contacting a plant, seed, or plant growth medium with the biostimulant composition described herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts a schematic labeling of components of a ZST system with a single reactor.

FIG. 2 is an exemplary schematic emphasizing the floc flights present in the clarifier of the NTS 2.0 system.

FIG. 3 is an exemplary schematic depicting the PST system.

FIG. 4 depicts a serialized PST system (PST-sIP) with a series of reactors and inputs to the bioreactor system.

FIG. 5 depicts a serialized ZST system featuring labeling of a series of reactors and other components.

FIG. 6 is an exemplary schematic labelling the parts of a NTS bioreactor system.

FIG. 7 depicts another serialized ZST system featuring a complete mixed reactor and a series of fluidized bed reactors.

FIG. 8 depicts another serialized ZST system featuring a complete mixed reactor and a series of packed bed reactors with scaffolding.

FIG. 9 is a graph showing the comparison of solubilized Zn2+ (in mg/L) in solution between the ZST 2.0 fluidized bed reactor (FBR) and the ZST 2.0 packed bed reactor (PBR) across different phases.

FIG. 10 is a graph showing the comparison of dissolved oxygen (in mg/L) between the ZST fluidized bed reactor (FBR) and the ZST packed bed reactor (PBR) across different phases.

FIGS. 11A-11B are a series of graphs showing measures of Zn uptake and plant growth in navy beans treated with MAP fertilizer coated with one of four different treatments: water (UTC), Circa™ Zn, MS4666, and MS4666 with Circa™ Zn. FIG. 11A shows the amount of zinc uptake (ug/plant) for navy beans. FIG. 11B shows total biomass, shoot dry weights, and root dry weights across conditions.

FIG. 12 is a graph showing results of measuring zinc uptake per corn plant treated with MAP fertilizer coated with one of four different treatments: water (UTC), Circa™ Zn, MS4666, and MS4666 with Circa™ Zn.

FIGS. 13A-13B are a series of graphs showing evaluation of Zn uptake and crop growth promotion in corn treated with MAP fertilizer coated with one of four different treatments: water (UTC), Circa™ Zn, MS4666, and MS4666 with Circa™ Zn. FIG. 13A shows results of measuring zinc uptake per plant for each condition. FIG. 13B shows total biomass, shoot dry weights, and root dry weights across conditions.

FIG. 14 is a graph showing the uptake of nitrogen, phosphorus, and potassium (μg/plant) in corn treated with MAP fertilizer coated with one of four different treatments: water (UTC), Circa™ Zn (CZN), MS4666, and MS4666 with CZN. For each macronutrient condition, left to right bars are: UTC, CZN, MS4666, MS4666+CZN.

FIG. 15 is a graph showing plant growth promotion (measured as dry weights (g)) in corn fertilized with MAP fertilizer coated with one of four different treatments: water (UTC), Circa™ Zn, MS4666, and MS4666 with Circa™ Zn. Dry weights were measured for the total biomass, shoot, and root. For each treatment condition, left to right bars are: root, stem, and total biomass.

FIG. 16 shows the differences in Arabidopsis growth (in average leaf area (cm²)) across intact and filter-sterilized (F/S) solutions for all ZST systems. UTC and Accomplish LM are used as controls.

FIGS. 17A-17C depict effects of different treatments on the dry biomass of corn. Percentages above the bars denote the percent change from UTC. An asterisk denotes significant difference from UTC. FIG. 17A shows the percent change of total dry biomass from UTC across multiple treatments. FIG. 17B depicts the average shoot dry biomass. FIG. 17C depicts the average root dry biomass.

FIGS. 18A-18C are a series of graphs showing the effects of the loading input components and supernatant (SPN) of the ZST system on plant growth production in corn. FIG. 18A shows total biomass treated with loading inputs and ZST 1.5 SPN. FIG. 18B shows total biomass with fertilizer, loading inputs plus Circa™ Zn. and ZST 1.5 SPN plus Circa™ Zn. FIG. 18C shows total biomass with of Circa™ Zn alone, ZST 1.5 SPN alone, and ZST 1.5 SPN plus Circa™ Zn.

FIGS. 19A-19B are a series of graphs depicting zinc uptake (in ug/plant) in corn after treatment with ZST SPN or isolate input (MS4666) compared to UTC and Circa™ Zn (CZN). FIG. 19A shows zinc uptake from the input into the ZST system (MS4666) and ZST SPN compared to UTC and CZN alone. FIG. 19B shows zinc uptake from the isolate input into the ZST system in combination with CZN (MS4666 CZN) and from ZST 1.5 SPN with CZN.

FIG. 20 shows a series of graphs depicting total corn grain yield (in g) across different ZST systems, untreated control (UTC), and Circa™ Zn (CZN) fertilizer alone.

FIG. 21 depicts the total zinc (μg) measured per ear of corn across different ZST systems, untreated control (UTC), and Circa™ Zn (CZN) fertilizer alone and in combination with ZST systems.

FIGS. 22A-22B are a series of graphs depicting the total macronutrients (nitrogen, phosphorus, and potassium) measured in mg per ear of corn across different ZST systems, untreated control (UTC), and Circa™ Zn (CZN) fertilizer alone. FIG. 22A shows results for ZST systems without CZn. FIG. 22B shows results for CZN alone and ZST systems with CZn. For each condition, left to right bars are: UTC, ZST 1.5, ZST 2.0 FBR, and ZST 2.0 PBR.

FIGS. 23A-23B are tables showing macronutrient and micronutrient content in the corn grain across ZST treatment systems using an untreated control (UTC) without CZN fertilizer and all ZST treatments (FIG. 23A) and using CZN fertilizer and all ZST treatments (FIG. 23B).

FIGS. 24A-24C are a series of graphs with harvest yield for three vegetables: broccoli (FIG. 24A), cabbage (FIG. 24B), and lettuce (FIG. 24C), measured as boxes/acre.

FIGS. 25A-25C show a series of graphs with plant growth promotion results of MS4666. Treatment with MS4666 shows superior corn yield compared to that from untreated controls (FIGS. 25A-25B). Treatment with MS4666 at a rate of 2.0 qt/acre shows superior soybean yield compared to that from untreated controls (FIG. 25C).

FIG. 26 shows a distance-based neighbor joining phylogenetic tree based on 16s rRNA gene.

FIG. 27 shows results from a zinc solubilization capacity in vitro assay between the target isolate MS4666, PST Floc, and ZST-1.0 Batch solutions using PST Floc (Batch-A) or PST Floc with MS4666 (Batch-B).

FIG. 28 shows a graph depicting concentrations of total bacteria and Zn-solubilizers across ZST-1.5 process and base product.

FIG. 29 shows measurements of Zn2+ made soluble from ZST-1.5 Reactor 1 (R1) and ZST-1.5 Base Product. * indicates p<0.0001.

FIG. 30 shows concentration of Zn2+found in ZST-2.0 FBR and ZST-2.5 reactors and base product.

FIG. 31 shows results measuring Zn2+ made soluble in ZET experiment from each treatment condition.

FIGS. 32A-32B depict experimental results of MS2839 and MS1835 spore counts following seven or fourteen days of incubation. Treatments were separated based on normal feeding (e.g., N Feed) or supplemental feeding (e.g., S Feed). FIG. 32A shows results of MS2839 and FIG. 32B shows results of MS1835.

FIGS. 33A-33B depicts box plot target counts of MS1835 (FIG. 33A) or MS2839 (FIG. 33B). Treatments used either base product of the P2 digestion system or water.

FIGS. 34A-34B depicts box plot target counts of MS1835 (FIG. 34A) or MS2839 (FIG. 34B). Treatments used either liquid or powder spore preparation as the inoculation medium.

FIGS. 35A-35B depicts box plot target counts of MS1835 (FIG. 35A) or MS2839 (FIG. 35B). Treatments comprised either treatments with additional supplemented feeding or treatments that received normal feeding.

FIG. 36 is a graph showing the plant growth production of Arabidopsis (measured in average leaf area, cm²) for UTC (untreated control) and PST supernatant.

FIG. 37 is a graph depicting corn shoot dry biomass (in grams) across UTC and PST 2.0 sIP. Results are from experiments with corn which was fertilized with PST 2.0 sIP coated monoammonium phosphate (MAP).

FIG. 38 is a graph depicting corn shoot dry biomass (in grams) across UTC and PST 2.0 sIP. Results are from experiments in which corn was treated via in-furrow application.

FIGS. 39A-39C are graphs showing the effects of treatment of corn with MAP coated with PST-sIP supernatant, PST supernatant, or no coating (UTC) on plant height (FIG. 39A), leaf area (FIG. 39B), and leaf chlorophyll content (FIG. 39C).

FIG. 40 is a graph depicting uptake of macronutrients, such as nitrogen, sulfur, phosphorus, potassium, magnesium, and calcium, in corn shoots (represented as % UTC, mg/shoot) after treatment with PST-sIP product at 2 qt/t rate. All results are from corn which was fertilized with MAP coated with PST-sIP product.

FIG. 41 is a graph depicting uptake of micronutrients, such as boron, zinc, manganese, copper, and iron in corn shoots (represented as % UTC, ug/shoot) after treatment with PST-sIP product at 2 qt/t rate. All results are from corn which was fertilized with MAP coated with PST-sIP product.

FIG. 42 is a graph showing uptake of macronutrients in corn shoots (represented as % UTC, mg/shoot) across PST-sIP. Results are from an experiment in corn in which the treatments were applied in-furrow at planting. Bars from left to right for each system are: nitrogen (N), sulfur(S), phosphorus (P), potassium (K), magnesium (Mg), and calcium (Ca). All results are from corn which was treated with PST-sIP product in-furrow at 4 qt/acre application rate.

FIG. 43 is a graph showing uptake of micronutrients in corn shoots (represented as % UTC, ug/shoot) across PST-sIP. Results are from a corn experiment in which the treatments were applied in-furrow at planting. Bars from left to right for each system are: boron (B), zinc (Zn), manganese (Mn), iron (Fe), and copper (Cu). All results are from corn which was treated with PST-sIP product in-furrow at 4 qt/acre application rate.

FIGS. 44A-44C are graphs showing the effects of treating corn in-furrow with PST-sIP supernatant, PST supernatant, or untreated control on leaf area (FIG. 44A), stem diameter (FIG. 44B), and leaf chlorophyll content (FIG. 44C).

FIG. 45 is a graph showing MS2839 spore count (cfu/ml) in supernatant for different ratios of floc. Bars from left to right for each percent floc condition are: Time Zero, 1 HR, 6 HR, 24 HR, and 48 HR post-inoculation.

FIG. 46 is a graph showing MS2839 counts (cfu/ml) from PST-sIP digestion system over time in supernatant (BP) and WB.

FIG. 47 is a graph showing FIG. 27 shows counts of MS2839 (cfu/ml) from PwST-sIP digestion system over time with or without floc-folding flights. T.L. denotes trend-line for each condition.

FIG. 48 shows an exemplary schematic of a NTS system with packed bed reactors.

FIG. 49 shows an exemplary schematic of a NTS system with fluidized bed reactors.

FIG. 50 is an exemplary schematic of a NTS 2.0 system with floc flights present in the clarifier.

FIGS. 51A-51B are a series of graphs showing leaf chlorophyll contents across treatment conditions. FIG. 51A shows results from V5 growth stage in corn. FIG. 51B shows results from V8 growth stage in corn.

FIG. 52 is a graph depicting the photosynthetic quantum yield across treatment conditions tested in corn at V9 growth stage. NTS treatments at both applications rates and isolates showed increased photosystem light capture efficiency.

FIG. 53 is a graph depicting photosynthetic electron transport rate across treatment conditions in corn at V9 growth stage.

FIG. 54 is a graph depicting the number of corn tillers per corn plant across treatment conditions, measured at V5 growth stage.

FIG. 55 is a graph depicting corn plant height across treatment conditions, measured at V7 growth stage.

FIG. 56 is a graph depicting the stem diameter across treatment conditions.

FIG. 57 is a graph depicting the stomatal conductance (the rate of CO₂ and H₂O gas exchange) across treatment conditions in corn, at V9 growth stage.

FIG. 58 is a graph depicting the transpiration rate (efficiency of water movement into and through the plant) across treatment conditions, measured at V9 growth stage in corn.

FIG. 59 is a graph depicting the corn dry biomass, measured as dry shoot weight, across treatment conditions.

FIG. 60 is a graph depicting results of acetylene reduction in corn root/basal stem, measured across treatment conditions.

FIG. 61 shows results from the sorghum grass study testing shake flasks using isolates with different aeration. All treatments showed significantly more leaf area than that of untreated control (UTC). ANOVA student's t-test (p<0.05) Levels not connected by same letter are significantly different.

FIG. 62 shows average pixel shoot area when shake flasks performance is grouped by aeration conditions.

FIG. 63 shows average performance of shake flasks when different ratios of PST WB are used. Shake flask treatments were grouped based on the ratio of PST WB used under both aerobic and anaerobic conditions.

FIGS. 64A-64B show exemplary schematics of the NTS batch systems. FIG. 64A shows the prototype 1 (e.g., PT1) system. FIG. 64B shows the prototype 2 (e.g., PT2) system.

FIG. 65 shows the effects of products from the NTS batch systems (e.g., PT1 and PT2) on ethylene production. PT1 product shows the greatest ethylene peak compared to that from UTC and PT2.

FIGS. 66A-66B show two graphs with results from a plant growth promotion test of the PT1 and PT2. Products of the NTs batch system were applied at 2 qt/acre or 4 qt/acre. On both Day 10 (FIG. 66A) and Day 14 (FIG. 66B), PT2 showed the greatest leaf area compared to that from PT1.

FIG. 67 shows results from an Arabidopsis plant growth promotion rockwool test for NTS 1.1,1.2, 1.3 and 1.4 lines with three reactors. All treatments showed numerically greater average leaf area measurement from the UTC with all having significant plant growth promotion (p=0.0008).

FIG. 68 shows results across NTS 1.0 systems with either three or four reactors in the system. There was a general increase of performance in the output solutions when the retention time of the system was extended by about 42.86%.

FIG. 69 shows the plant growth promotion of intact NTS solutions and metabolites. Intact NTS 1.0 system solutions and their metabolites showed increased average leaf area compared to that from UTC plants.

FIG. 70 shows Arabidopsis plant growth in NTS 2.2 and NTS 2.3 systems with intact solution or metabolites. All NTS 2.0 system treatments showed greater average leaf area than untreated control.

FIGS. 71A-71B show Arabidopsis plant growth in NTS 1.4 and NTS 1.5 systems with intact solution or metabolites. FIG. 71A shows the results from intact solution treatments. FIG. 71B shows the results from metabolite treatments. All NTS system treatments showed greater average leaf area than untreated control, with NTS 1.4 BP intact and metabolite showing the greatest plant growth promotion.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Described herein are systems and methods that employ microbial digestion of various feedstocks. A system of the present disclosure may comprise a continuous system capable of serialized isolate production (e.g., sIP system). The isolate production of the sIP system can occur within a mixed consortium of microbes. The target isolates of a sIP bioreactor system may become enriched in the microbial environment and may demonstrate improved efficacy and functionality. A targeted functionality may be zinc solubilization of bound zinc from soil or fertilizer and improved nutrient uptake in plants. A targeted functionality may be phosphate solubilization of insoluble phosphate from soil or fertilizer and improved nutrient uptake in plants. A targeted functionality may be nitrogen use efficiency and/or nitrogen fixation from soil or fertilizer and improved nutrient uptake in plants. A target isolate may possess commercially valuable properties and can be introduced into a continuous (e.g., serialized) reactor system comprised of a complex microbial consortia that has been modified for functionality (e.g., for zinc solubilization, phosphate solubilization, nitrogen use efficiency, etc.). Without wishing to be bound by theory, a target isolate may provide a performance benefit to a microbial community of the digestions systems described herein, providing a chemical and/or functional synergistic relationship as it grows in the system. A bioreactor system described herein may comprise a zinc solubilization technology (ZST) system, with a target function to enrich a population or populations of zinc-solubilizing microbes, metabolites, or any combination thereof. A bioreactor system described herein may comprise a phosphate solubilization technology (PST) system, with a target function to enrich a population or populations of phosphate-solubilizing microbes, metabolites, or any combination thereof. A bioreactor system described herein may comprise a nitrogen use efficiency (NUE) system, with a target function to enrich a population or population of nitrogen use efficiency and/or nitrogen fixation microbes, metabolites, or any combination thereof.

The products of digestion methods and systems described herein can comprise microbes and metabolites produced by microbial digestion of feedstock substrates. In some embodiments, the products of digestion methods and systems described herein can comprise biostimulant compositions that have plant growth promoting properties when applied to horticultural items such as plants, plant parts (e.g., shoots, stems, leaves, buds, flowers, leaf axils, roots), seeds, or a medium in which plants grow (e.g., soil). In some embodiments, methods and systems described herein are arranged to selectively promote growth of microbes that have a desired plant growth promoting property themselves or that produce metabolites that have the desired plant growth promoting property, such that the biostimulant product has the desired plant growth promoting property. Applications of the products of the bioreactor systems described herein may be on dry-fertilizers, applied in conjunction with the application of fertilizers, in formulations with additional components including liquid fertilizers, micronutrient coating formulations, foliar applications, or any combination thereof. Applications of the products of the bioreactor systems described herein may be to a part of a plant, such as a shoot, a stem, a leaf, a lateral bud, a terminal bud, a flower, a leaf axil, a root (e.g., a primary root, a lateral root, a root hair, a root cap), or any combination thereof. These and other features of embodiments disclosed herein are described in more detail below.

### I. Definitions

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” comprise plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The term “culturing”, as used herein, can refer to the propagation of organisms on or in media of various kinds. Non-limiting examples of suitable media comprise tryptic soy agar (TSA), zinc agar, phosphate solubilizing medium which contain insoluble forms of phosphate, nutrient medium, lysogeny broth (LB medium), plate count agar, or any combination thereof.

As used herein, the term “enriched culture” of an isolated microbial strain can refer to a microbial culture wherein the total microbial population of the culture contains a percentage of a target isolated strain. An enriched culture may comprise a percentage of a target isolated strain and a population of microbes enriched for a particular functionality (e.g., zinc solubilization, nitrogen use efficiency, phosphate solubilization). In some embodiments, an enriched culture may comprise a percentage of a target isolated strain, a population of microbes enriched for a particular functionality, and metabolites enriched for a particular functionality (e.g., zinc solubilization, nitrogen use efficiency, phosphate solubilization). The enriched culture may comprise a percentage of a total bacteria population in a container of bioreactor system described herein. The enriched culture may comprise a percentage of a total bacteria population in an output product (e.g., biostimulant) described herein. An enriched culture may comprise an increased amount of a target isolated strain and/or a target population of microbes compared to a total microbial population of a culture. An enriched culture may comprise a growing population of a target isolated strain and a population of microbes enriched for a particular functionality (e.g., zinc solubilization, nitrogen use efficiency, phosphate solubilization) over a time period. In some embodiments, an enriched culture can refer to a microbial culture wherein the total microbial population of the culture contains at least about 0.001%, at least about 0.01%, at least about 0.05%, at least about 0.1%, at least about 0.5%, at least about 1%, at least about 1.5%, at least about 2%, at least about 2.5%, at least about 3%, at least about 4%, at least about 5%, at least about 10%, at least about 15%, at least about 20%, at least about 25%, at least about 30%, at least about 40%, at least about 50%, at least about 60%, at least about 70%, at least about 75%, at least about 80%, or at least about 90% of a target isolated strain, a population of microbes enriched for a particular functionality, metabolites enriched for a particular functionality, or any combination thereof. In some embodiments, an enriched culture can refer to a microbial culture wherein the total microbial population of the culture contains at most about 90%, at most about 80%, at most about 75%, at most about 70%, at most about 60%, at most about 50%, at most about 40%, at most about 30%, at most about 25%, at most about 20%, at most about 15%, at most about 10%, at most about 5%, at most about 4%, at most about 3%, at most about 2.5%, at most about 2%, at most about 1.5%, at most about 1%, or at most about 0.5% of a target isolated strain, a population of microbes enriched for a particular functionality, metabolites enriched for a particular functionality, or any combination thereof. In some embodiments, an enriched culture can refer to a microbial culture wherein the total microbial population of the culture contains from about 0.5% to about 75% of a target isolated strain, a population of microbes enriched for a particular functionality, metabolites enriched for a particular functionality, or any combination thereof. In some embodiments, an enriched culture can refer to a microbial culture wherein the total microbial population of the culture contains from about 0.5% to about 1%, about 0.5% to about 2%, about 0.5% to about 3%, about 0.5% to about 5%, about 0.5% to about 10%, about 0.5% to about 15%, about 0.5% to about 20%, about 0.5% to about 25%, about 0.5% to about 50%, about 0.5% to about 60%, about 0.5% to about 75%, about 1% to about 2%, about 1% to about 3%, about 1% to about 5%, about 1% to about 10%, about 1% to about 15%, about 1% to about 20%, about 1% to about 25%, about 1% to about 50%, about 1% to about 60%, about 1% to about 75%, about 2% to about 3%, about 2% to about 5%, about 2% to about 10%, about 2% to about 15%, about 2% to about 20%, about 2% to about 25%, about 2% to about 50%, about 2% to about 60%, about 2% to about 75%, about 3% to about 5%, about 3% to about 10%, about 3% to about 15%, about 3% to about 20%, about 3% to about 25%, about 3% to about 50%, about 3% to about 60%, about 3% to about 75%, about 5% to about 10%, about 5% to about 15%, about 5% to about 20%, about 5% to about 25%, about 5% to about 50%, about 5% to about 60%, about 5% to about 75%, about 10% to about 15%, about 10% to about 20%, about 10% to about 25%, about 10% to about 50%, about 10% to about 60%, about 10% to about 75%, about 15% to about 20%, about 15% to about 25%, about 15% to about 50%, about 15% to about 60%, about 15% to about 75%, about 20% to about 25%, about 20% to about 50%, about 20% to about 60%, about 20% to about 75%, about 25% to about 50%, about 25% to about 60%, about 25% to about 75%, about 50% to about 60%, about 50% to about 75%, or about 60% to about 75% of a target isolated strain, a population of microbes enriched for a particular functionality, metabolites enriched for a particular functionality, or any combination thereof.

The term “composition” as used herein can refer to a combination of an active agent (e.g., a microbial strain described herein) and at least one other compound, carrier, or composition, which can be inert (for example, a detectable agent or liquid carrier) or active, such as, but not limited to, a fertilizer, nutrient, or pesticide. A microbial composition refers to a composition comprising at least one microbial species. A composition may comprise microbial metabolites generated in a microbial consortium of a bioreactor system described herein.

An “effective amount”, as used herein, can refer to an amount sufficient to effect beneficial and/or desired results. An effective amount can be administered in one or more administrations. The expression “effective microorganism” used herein in reference to a microorganism is intended to mean that the subject strain exhibits a degree of promotion of plant health, growth and/or yield, at a statistically significant level, that of an untreated control. In some instances, the expression “an effective amount” is used herein in reference to that quantity of microbial treatment which can be used to obtain a beneficial or desired result relative to that occurring in an untreated control under suitable conditions of treatment as described herein. For example, the expression “an agriculturally effective amount” is used herein in reference to that quantity of microbial treatment which can be used to obtain an agriculturally beneficial or desired result relative to that occurring in an untreated control under suitable conditions of treatment as described herein. The effective amount of an agricultural formulation or composition that may be applied for the improvement of plant health, growth and/or yield, can be readily determined.

A “carrier” as used herein can refer to a substance or a composition that support the survival of the microbes. Such carriers may be either organic or non-organic.

“Percentage of sequence identity”, as used herein, can be determined by comparing two optimally locally aligned sequences over a comparison window defined by the length of the local alignment between the two sequences. The amino acid sequence in the comparison window may comprise additions or deletions (e.g., gaps or overhangs) as compared to the reference sequence (which does not comprise additions or deletions) for optimal alignment of the two sequences.

Local alignment between two sequences may comprise segments of each sequence that are deemed to be sufficiently similar according to a criterion that depends on the algorithm used to perform the alignment (e.g., BLAST). The percentage of sequence identity is calculated by determining the number of positions at which the identical nucleic acid base or amino acid residue occurs in both sequences to yield the number of matched positions, dividing the number of matched positions by the total number of positions in the window of comparison and multiplying the result by 100. Optimal alignment of sequences for comparison may be conducted by the local homology algorithm of Smith and Waterman (Add. APL. Math. 2:482, 1981), by the global homology alignment algorithm of Needleman and Wunsch (J Mol. Biol. 48:443, 1970), by the search for similarity method of Pearson and Lipman (Proc. Natl. Acad. Sci. USA 85:2444, 1988), by heuristic implementations of these algorithms (NCBI BLAST, WU-BLAST, BLAT, SIM, BLASTZ), or by inspection. Given that two sequences have been identified for comparison, GAP and BESTFIT may be employed to determine their optimal alignment. Typically, the default values of 5.00 for gap weight and 0.30 for gap weight length are used. The term “substantial sequence identity” between polynucleotide or polypeptide sequences refers to polynucleotide or polypeptide comprising a sequence that has at least about 50% sequence identity, at least about 60% sequence identity, at least about 70% sequence identity, at least about 80% sequence identity, at least about 85% sequence identity, at least about 90% sequence identity, at least about 95% sequence identity, at least about 96% sequence identity, at least about 97% sequence identity, at least about 98% sequence identity, or at least about 99% sequence identity compared to a reference sequence using the programs. In addition, pairwise sequence homology or sequence similarity, as used, refers to the percentage of residues that are similar between two aligned sequences. Families of amino acid residues having similar side chains have been well defined in the art. These families comprise amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Query nucleic acid and amino acid sequences can be searched against subject nucleic acid or amino acid sequences residing in public or proprietary databases. Such searches can be done using the National Center for Biotechnology Information Basic Local Alignment Search Tool (NCBI BLAST v 2.18) program. The NCBI BLAST program is available on the internet from the National Center for Biotechnology Information (blast.ncbi.nlm.nih.gov/Blast.cgi). Typically the following parameters for NCBI BLAST can be used: Filter options set to “default”, the Comparison Matrix set to “BLOSUM62”, the Gap Costs set to “Existence: 11, Extension: 1”, the Word Size set to 3, the Expect (E threshold) set to 1e-3, and the minimum length of the local alignment set to 50% of the query sequence length. Sequence identity and similarity may also be determined using GenomeQuest™ software (Gene-IT, Worcester Mass. USA).

The term “plant growth promotion” (PGP) can refer to processes that can promote plant health, growth, yield, or any combination thereof. In some embodiments, PGP can encompass a wide range of improved plant properties, including but not limited to, improved nitrogen fixation, improved phosphate uptake, improved zinc uptake, improved root development, increased leaf area, increased plant yield, increased uptake of macronutrients, increased uptake of micronutrients, increased seed germination, enhancing seed germination, enhancing early plant development, improving root growth, improving shoot growth, improving plant height, increasing nutrient uptake, mitigating transplant shock, improving plant reproduction, improving soil microbial activity, increased photosynthesis, increased abundance of functional enzymes, increased dry biomass, or an increase in the accumulated biomass of the plant. In some embodiments, the microbial strains, isolates, cultures, compositions, or synthetic consortia as described herein improve nutrient uptake, plant health and vigor, improve root development, increase leaf area, increase plant yield, increased uptake of macronutrients, increased uptake of micronutrients, increase seed germination, increased abundance of functional enzymes, increased dry biomass, or an increase in the accumulated biomass of the plant. In some embodiments, the microbial strains, isolates, cultures, or compositions as described herein increase the size or mass of a plant or parts thereof, as compared to a control plant, or a plant that has not been treated with a substance, or parts thereof or as compared to a predetermined standard. In some embodiments, the microbial strains, isolates, cultures, compositions, or synthetic consortia as described herein improve the health, vigor and yield of a plant, as compared to a control plant or a plant that has not been treated with a substance, but also can survive and multiply in microhabitats associated with the root surface.

Microbes described herein as “zinc-solubilizing microbes” and “zinc-solubilizing bacteria” or “zinc solubilizers” may refer to a microbe or bacterium that either (a) has a measurable increase in zinc made soluble relative to a control in an assay that comprises suspending adding a test solution that comprises the microbe or bacterium to media with insoluble, inorganic zinc, incubating the media, and measuring an amount of insoluble zinc released into solution and made soluble using an instrument such as a Inductively Couples Plasma (ICP) Atomic Emission Spectrometer and comparing to known standards, or (b) makes a visible halo when grown in a colony on a plate with insoluble zinc.

Microbes described herein as “phosphate-solubilizing microbes” and “phosphate-solubilizing bacteria” or “phosphate solubilizers” may refer to a microbe or bacterium that either (a) has a measurable increase in phosphate made soluble relative to a control in an assay that comprises suspending adding a test solution that comprises the microbe or bacterium to media with insoluble, inorganic phosphate (e.g., tricalcium phosphate (Ca₃(PO₄)₂), incubating the media, and measuring an amount of insoluble phosphate released into solution and made soluble using an instrument such as a SEAL AQ400 and comparing to known standards, or (b) makes a visible halo when grown in a colony on a plate with insoluble phosphate.

Microbes described herein as “nitrogen use efficiency microbes” and “nitrogen use efficiency bacteria” may refer a microbe or bacterium that (a) has a measurable increase in nitrogen fixation (conversion of molecular dinitrogen (N₂) into ammonia (NH₃)) relative to a control in an assay that comprises adding a test solution that comprises the microbe or bacterium to media, incubating the media, and measuring an amount of molecular dinitrogen and ammonia using an instrument such as a SEAL AQ400 and comparing to known standards, (b) is identified via an acetylene reduction assay as described herein, (c) is identified based on nifh gene quantification (e.g., the microbe may have a nifh gene), (c) recruits nitrogen fixers to the root zones or other tissues of plants, increases organic nitrogen content, in the soil, or increases mineralization of organic nitrogen in the soil, or (d) enables plant growth in nitrogen concentrations that normally reduce growth compared to that of a control (e.g., non-nitrogen use efficiency microbe).

As used herein, the term “yield” can refer to the amount of harvestable plant material or plant-derived product, and is normally defined as the measurable produce of economic value of a crop.

For crop plants, “yield” can also mean the amount of harvested material per acre or unit of production. Yield may be defined in terms of quantity or quality. The harvested material may vary from crop to crop, for example, it may be seeds, above ground biomass, roots, fruits, cotton fibers, any other part of the plant, any plant-derived product which is of economic value, or any combination thereof.

In some embodiments, microbial strains, isolates, cultures, compositions, biostimulant compositions, or any combination thereof described herein may be used to promote improved crop or product quality, including food quality. Improving crop quality may comprise improving characteristics that make a crop or product more marketable such as, for example, a desired color, size, or shape. Improving crop quality may also including making a food product with desired nutritional characteristics, nutraceutical characteristics, pharmaceutical characteristics, or any combination thereof. Improving crop or product quality may comprise adding valuable attributes that may increase price. Improving crop or product quality may comprise increasing value per harvest unit, such as, for example, providing increased oil, sugar, starch, or protein yield per unit weight of harvested crop or product.

In some embodiments, the microbial strains, isolates, cultures and compositions according to the embodiments of this application can lead to plant growth promotion or plant growth improvement that is an at least 5% increase, at least 10% increase, at least 25% increase, at least 50% increase, at least 75% increase, or at least a 100% increase in the property being measured. In some embodiments, the microbial strains, isolates, cultures and compositions according to the embodiments of this application lead to plant growth promotion or plant growth improvement that is at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, at least about 96%, at least about 97%, at least about 98%, at least about 99%, or a 100% increase in the property being measured. In some embodiments, the microbial strains, isolates, cultures and compositions of this application may produce an above stated percentage increase in zinc uptake, an above stated increase in zinc solubilization capacity, an above stated percentage increase in phosphate uptake, an above stated increase in phosphate solubilization capacity, an above stated percentage increase in nitrogen fixation, an above stated increase in nitrogen content, an above stated increase in nitrogen acquisition, an above stated increase in nitrogen uptake, or an above stated increase in total root weight, leaf area, or plant product yield (e.g., an above stated percentage increase in plant product weight).

A “control plant”, as used herein, can provide a reference point for measuring changes in phenotype of the subject plant, and may be any suitable plant cell, seed, plant component, plant tissue, plant organ or whole plant. A control plant may include, but is not limited to, (a) a plant which is genetically identical to the subject plant but which is not exposed to the same treatment (e.g., inoculant treatment) as the subject plant or (b) the subject plant itself, under conditions in which it has not been exposed to a particular treatment such as, for example, an inoculant or combination of inoculants, a biostimulant composition, other chemicals, or any combination thereof. A treated plant may comprise a plant that has had an inoculum of a microbe and/or a biostimulant composition as described herein applied to any part of the plant (e.g., seed, stem, root, shoot, leaf, or any combination thereof). A treated plant may comprise a plant that has had an inoculum of a microbe or a biostimulant composition as described herein applied using an in-furrow application. A treated plant may comprise a plant that has had an inoculum of a microbe and/or a biostimulant composition as described herein applied using a side-dress application, a broadcast application, a y-drop application, a tape application, a fertigation application, or any combination of applications thereof. A treated plant may comprise a plant which has had an inoculum of a microbe or a biostimulant composition as described herein applied to the seed of the plant. A treated plant may comprise a plant that has had an inoculum of a microbe or a biostimulant composition as described herein applied to the soil. An untreated plant may comprise a plant that that has not had an inoculum of a microbe or a biostimulant composition as described herein applied directly or indirectly.

“Inoculant” as used herein can refer to any culture or preparation that comprises at least one microorganism. In some embodiments, an inoculant (sometimes referred to as a microbial inoculant or a soil inoculant) is an agricultural addition that uses beneficial microbes (including but not limited to endophytes) to promote plant health, growth and/or yield. Many of the microbes suitable for use in an inoculant form symbiotic relationships with the target crops where both parties benefit (mutualism). For example, an isolated microbial strain as described herein may benefit from carbon sources from the roots of a plant and the plant may benefit from metabolites generated by metabolism of the microbe. Without wishing to be bound by theory, a plant may be colonized by the isolate, and the colonization of the roots may block plant pathogens from accessing the roots and/or provide plant growth and/or health benefits to the plant. An inoculant (e.g., inoculum of a microbe/microbial strain) can be added at one time point during an operating of a bioreactor system process. An inoculant (e.g., inoculum of a microbe/microbial strain) can be added at two or more time points during an operating of a bioreactor system process.

The term “serialized isolate production”, (sIP), can refer to specialized manipulated continuous serialized reactors that may enable the growth and enrichment of the microbes, isolates, target isolates, and/or microorganisms as described herein.

The term “reactor” can refer to a container of a system described herein that may hold a volume of fluid. The terms “reactor” and “container” can be used interchangeably.

The term “floc” can refer to a mass formed by the aggregation of a number of fine suspended particles. Particles can comprise biological particles, non-biological particles, or a combination thereof. For example, a floc can comprise organic materials recovered from a feedstock, waste, wastewater, sludge material, or any combination thereof of a fluid used in a bioreactor system. A floc can comprise biosolids and/or particles from digestion products of organic materials. Floc can comprise an aggregated mass of microorganisms (e.g., bacteria).

The term “whole broth” (WB) can refer to a blend of supernatant and floc at a ratio for use in the technologies as described herein. A whole broth may comprise microbial populations (e.g., zinc-solubilizing microbes, phosphate solubilizing microbes, nitrogen use efficiency-promoting microbes), enzymes, fungi, biosolids, or any combination thereof. For example, bacterial genera of a whole broth may comprise Haliscomenobacter, Lewinella, Caldilinea, Terrimonas, Acidobacterium, Lewinella cohaerens, Thauera phenylacetica, Thauera mechernichensis, Solitalea canadensis, Nitrospira moscoviensis, Kosakonia, Klebsiella, Rahnella, Kluyvera, Enterobacter, Achromobacter, Microbacterium, Gluconobacter, Methylobacterium, Pseudomonas, Pantoea, Azospirillum, Azocarus, Herbaspirillum, Burkholderia, Cyanobacteria, Bacillus, Paenibacillus, Kosakonia sacchari, Klebsiella variicola, Rahnella aquatilis, Kluyvera intermedia, Kosakonia pseusosacchari, Enterobacter spp., Achromobacter marplatensis, Azopirillum lipoferum, Microbacterium murale, Gluconobacter diazotrophicus, Methylobacterium symbioticum, Kosakonia sacchari, Klebsiella variicola, Rahnella aquatilis, Kluyvera intermedia, Kosakonia pseusosacchari, Enterobacter spp., Achromobacter marplatensis, Azopirillum lipoferum, Microbacterium murale, Gluconobacter diazotrophicus, Methylobacterium symbioticum, Paenibacillus borealis, Bacillus megaterium (Priestia megaterium), Bacillus amyloliquefaciens, Bacillus licheniformis, Paenibacillus sonchi, or any combination thereof. A whole broth may have plant growth promotion properties. For example, a whole broth may have zinc-solubilization capacity, phosphate-solubilization capacity, nitrogen-fixation capacity, or any combination thereof.

The terms “microbial consortium” or “microbial population” can refer to a group of microorganisms in an environment. Consortiums may be endosymbiotic or ectosymbiotic. Microorganisms in a microbial consortium can include, but are not limited to, bacteria, fungi, yeasts, lichens, algae, protozoa, archaea, molds, or any combination thereof.

The terms “supernatant,” “biostimulant product,” or “base product” can refer to the final product of the bioreactor system. The supernatant can be measured for amount of a microbial isolate, number of members within a microbial consortium, or types and amount of microbial metabolites with plant growth promotion capacity.

The term “load rate” can refer to a rate at which a source material is introduced into a bioreactor system. In some embodiments, load rate may refer to “organic load rate” or “hydraulic load rate”. An organic load rate can comprise a rate at which feedstock (e.g., organic feedstock) is introduced into the system. A hydraulic load rate can comprise a rate at which a hydraulic source is introduced into the system.

The term “internal recycle rate” can refer to a rate at which a working fluid is recycled within a phase space.

The term “hydraulic feed rate” can refer to a rate at which working fluid is transferred between phase spaces.

The term “hydraulic dwell time” can refer to an amount of time that a working fluid is present in a phase space.

The term “working fluid” can refer to a fluid substance supporting and transporting biology and nutrients through a system of containers. For example, a working fluid may comprise organic materials, microorganisms (e.g., microbes and/or metabolites), biosolids, macronutrients, micronutrients, organic nutrients, inorganic nutrients, or any combination thereof. A working fluid can comprise a solution that flows throughout a bioreactor system and may provide an enriched environment for microbes of the bioreactor system.

Whenever the term “at least,” “greater than,” or “greater than or equal to” precedes the first numerical value in a series of two or more numerical values, the term “at least,” “greater than” or “greater than or equal to” can apply to each of the numerical values in that series of numerical values. For example, greater than or equal to 1, 2, or 3 is equivalent to greater than or equal to 1, greater than or equal to 2, or greater than or equal to 3.

Whenever the term “at most,” “no more than,” “less than,” or “less than or equal to” precedes the first numerical value in a series of two or more numerical values, the term “at most,” “no more than,” “less than,” or “less than or equal to” can apply to each of the numerical values in that series of numerical values. For example, less than or equal to 3, 2, or 1 is equivalent to less than or equal to 3, less than or equal to 2, or less than or equal to 1.

II. Bioreactor systems for Production of Isolates

The present disclosure provides systems (e.g., bioreactor systems) with conditions to produce biostimulant products with plant-growth promoting capabilities (e.g., improved zinc solubilization capacity, improved phosphate solubilization capacity, promotion of efficient use of nitrogen).

Embodiments of systems and methods described herein can produce biostimulant products that may have a multi-modal way of promoting zinc uptake in plants. Embodiments of systems and methods described herein can produce biostimulant products that may have a multi-modal way of promoting phosphate uptake in plants. Embodiments of systems and methods described herein can produce biostimulant products that may have a multi-modal way of promoting efficient use of nitrogen by plants. Biostimulant products produced by embodiments described herein may be used to promote plant growth by applying the products to horticultural items such as plants, plant parts, seeds plant growth media (e.g., soil, fertilizer), or any combination thereof.

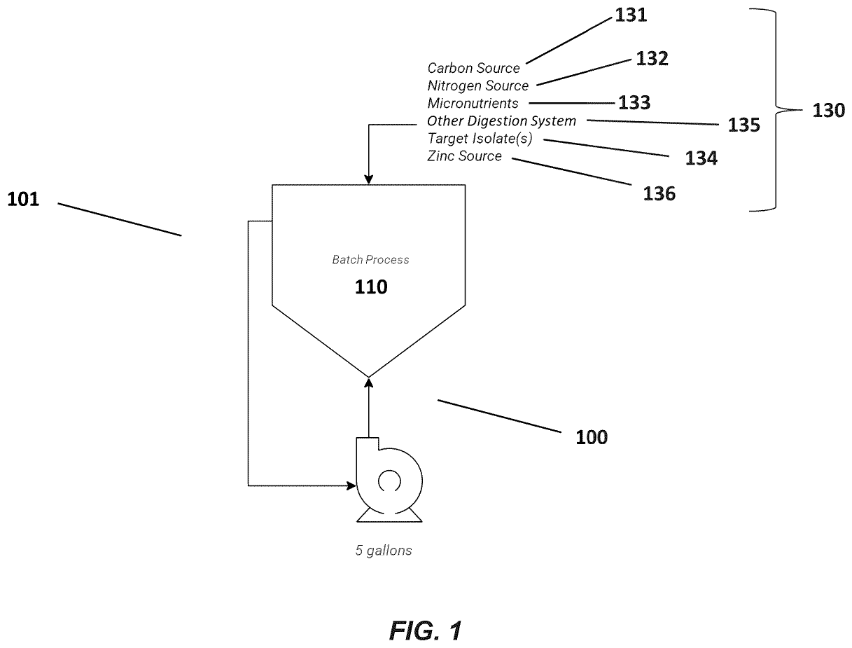

FIGS. 1-8 schematically illustrate examples of bioreactor systems 100 with conditions (e.g., microbes) that produce biostimulant products that may have a multi-modal way of promoting zinc solubilization, phosphate solubilization, and/or efficient use of nitrogen by plants.

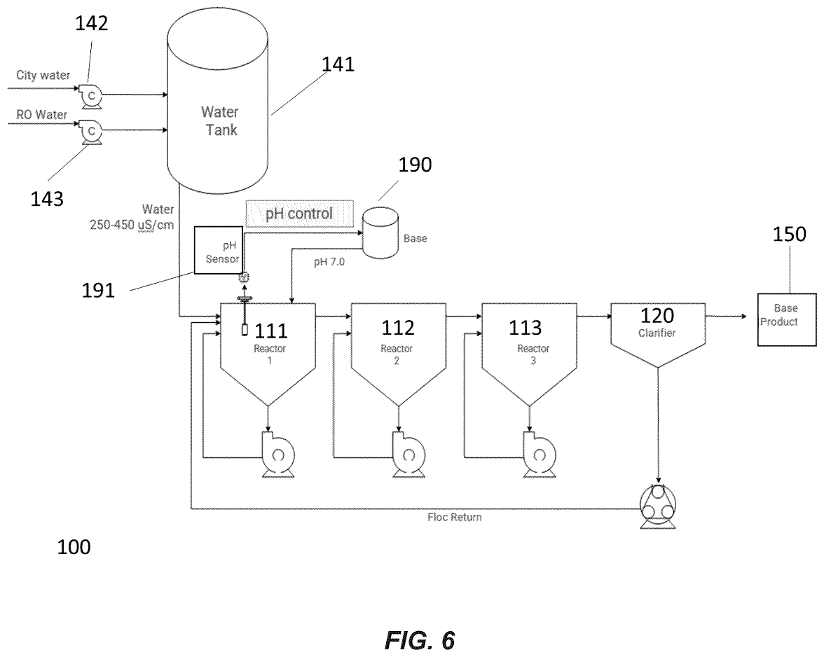

For example, in FIG. 6., the system 100 comprises a water tank 141 that comprises city water 142 and RO water 143. The system 100 also comprises a first reactor 111, a second reactor 112, a third reactor 113, and/or a clarifier container 120 connected sequentially in which feedstock can continuously flow and microbial consortia as described herein can be grown. The first reactor, second reactor, and/or third reactor may be packed bed reactors with a scaffolding within the reactors or may be fluidized bed reactors without the scaffolding. The water tank 141 is coupled to the first reactor 111 and provides continuous flow of water to the first reactor 111. The system 100 can also comprise an input channel that flows inputs into the bioreactor system. In some cases, the inputs into the bioreactor system 130 are flown into the first reactor through the water tank. In other cases, the input composition can be added to the first reactor. The inputs, as described herein, may comprise one or more of water 140, a microbial inoculum 134, nutrients 133, and/or a digestion substrate (e.g., aqueous organic feedstock).

In some embodiments, a reactor tank circulates working fluid within itself to recycle working fluid, wherein reactor tanks can comprise outflow pipes to circulate and recycle working fluid within each tank. Ports and piping between tanks can assist in transferring working fluid to adjacent reactor tanks. A working fluid in a final clarifier of a system may transfer from the reactor tank to a clarifier may produce a supernatant (e.g., base product). Working fluid flows through the serialized reactor system which can aid in selective growth of the added isolate and other microbes present that have the same property as the added isolate.

Base product from the clarifier can be accessed and further analyzed for microbial composition. In a bioreactor system described herein, a working fluid may flow from a mixing chamber through at least one reaction and to a clarifier container.

Parameters of the bioreactor system, such as flow rate and the solids content of the feedstock (e.g., organic feedstock), may be varied to achieve desired properties in the outflow biostimulant base product. In some embodiments, the hydraulic source is water. In some embodiments, the hydraulic source is a base product of another system. In some embodiments, the hydraulic source is a combination of water and a base product of another system. Water from the hydraulic source can be added to the feedstock (e.g., organic feedstock) of the bioreactor system to make an aqueous organic feedstock.

In some aspects, provided herein is a method of making a biostimulant composition. The biostimulant composition may comprise a plant growth promoting property (e.g., a desired plant growth promoting property). A bioreactor system may be provided. A bioreactor system comprising one or more containers (e.g., two or more containers) may be provided. A bioreactor system comprising one or more containers (e.g., two or more containers) arranged in a series may be provided. A container of the one or more containers (e.g., two or more containers) may comprise a volume of a working fluid. In some embodiments, each container of the one or more containers (e.g., two or more containers) may comprise a volume of a working fluid. The one or more containers (e.g., two or more containers) may comprise a first container, a second container, a third container, a fourth container, and the like. A container of the bioreactor system (e.g., a first container of the bioreactor system) may comprise a population of a microbial strain. The population of a microbial strain may comprise an established population of a microbial strain. The population of a microbial strain (e.g., the established population of a microbial strain) may have a plant growth promoting property (e.g., a desired plant growth promoting property).

In some embodiments, the bioreactor system may be operated for a duration of time. Operating the bioreactor system can comprise transferring an aqueous feedstock in a container (e.g., a first container). The aqueous feedstock can comprise a microbial consortium. Operating the bioreactor system can comprise transferring a portion of working fluid of a container (e.g., of the one or more containers) of the bioreactor system. Working fluid may be transferred out of a container of the two or more containers into a subsequent container. For example, a portion of working fluid may be transferred out of a second container into a third container. Working fluid may be transferred out of a container of the two or more containers as a product outflow stream. Operating the bioreactor system can comprise collecting at least a portion of working fluid. For example, at least a portion of the product outflow stream may be collected as the biostimulant composition.

The population of the microbial strain (e.g., the established population of the microbial strain) may not be present in the aqueous feedstock. The population of the microbial strain (e.g., the established population of the microbial strain) may not be present in the aqueous feedstock during the duration of time of operation. The population of the microbial strain (e.g., the established population of the microbial strain) may not be present in any other input to the bioreactor system (e.g., to a container of the bioreactor system). The population of the microbial strain (e.g., the established population of the microbial strain) may not be present in any other input to the bioreactor system (e.g., to a container of the bioreactor system) during the duration of time of operation. Any other input may comprise nutrients and/or digestion substrates (e.g., manure, biosolids, food waste, energy crops, or any combination thereof). In some embodiments, the established population of a microbial strain may not be present in the aqueous feedstock and/or any other input to the bioreactor system during a duration of operation at a concentration that is higher than 1% of the concentration of the established population of the microbial strain in the first container.

In some embodiments, operating the bioreactor system can comprise maintaining a concentration of the microbial strain. The concentration of the microbial strain may be maintained for the duration of time at which the bioreactor system is operating. A concentration of the microbial strain may be maintained in a container (e.g., a first container) at at least about 70%, at least about 75%, at least about 80%, at least about 85%, at least about 90%, at least about 95%, or greater than about 95% of a concentration of the microbial strain at the beginning of the duration of time. A concentration of the microbial strain may be maintained in a container (e.g., a first container) at at most about 95%, at most about 90%, at most about 85%, at most about 80%, at most about 75%, at most about 70%, or less than about 70% of a concentration of the microbial strain at the beginning of the duration of time. For example, a concentration of the microbial strain may be 1000 CFU/ml at the beginning of the duration of time and the concentration of the microbial strain may be maintained at 800 CFU/ml in a first container of the bioreactor system.

As an example, the present disclosure provides a method of making a biostimulant composition having a desired plant growth promoting property, the method comprising: (a) providing a bioreactor system comprising two or more containers arranged in a series, each of the two or more containers comprising a volume of a working fluid, wherein a first container comprises an established population of a microbial strain having the desired plant growth promoting property; (b) operating the bioreactor system for a duration of time by: (i) transferring into the first container an aqueous feedstock comprising a microbial consortium; (ii) transferring a portion of the working fluid out of each of the two or more containers into either a subsequent container of the bioreactor system or a product outflow stream; and (iii) collecting at least a portion of the product outflow stream as the biostimulant composition; wherein the established population of the microbial strain is not present in an aqueous feedstock or any other input in the bioreactor system during the duration of time at a concentration that is higher than 1% of the concentration of the established population of the microbial strain in the first container.

As another example, the present disclosure provides a method of making a biostimulant composition having a desired plant growth promoting property, the method comprising: (a) providing a bioreactor system comprising two or more containers arranged in a series, each of the two or more containers comprising a volume of a working fluid, wherein a first container comprises an established population of a microbial strain having the desired plant growth promoting property; (b) operating the bioreactor system for a duration of time by: (i) transferring into the first container an aqueous feedstock comprising a microbial consortium; (ii) transferring a portion of the working fluid out of each of the two or more containers into either a subsequent container of the bioreactor system or a product outflow stream; (iii) maintaining a concentration of the microbial strain throughout the duration of time in at least the first container at at least 80% of a concentration of the microbial strain at the beginning of the duration of time; and (iv) collecting at least a portion of the product outflow stream as the biostimulant composition; wherein the duration of time is at least 5 days; and wherein the microbial strain is not present in the aqueous feedstock or any other input into the bioreactor system during the duration of time at a concentration that is higher than 1% of the concentration of the microbial strain in the first container.

Maintaining the concentration of the microbial strain may comprise maintaining the concentration at at least about 10 CFU/ml, at least about 1×10² CFU/ml, at least about 5×10² CFU/ml, at least about 1×10³ CFU/ml, at least about 5×10³ CFU/ml, at least about 1×10⁴ CFU/ml, at least about 5×10⁴ CFU/ml, at least about 1×10⁵ CFU/ml, or greater than about 1×10⁵ CFU/ml. Maintaining the concentration of the microbial strain may comprise maintaining the concentration at at most about 1×10⁵ CFU/ml, 5×10⁴ CFU/ml, 1×10⁴ CFU/ml, 5×10³ CFU/ml, 1×10³ CFU/ml, 5×10² CFU/ml, 1×10² CFU/ml, 10 CFU/ml, or less than about 10 CFU/ml.

In some embodiments, maintaining the concentration of the microbial strain may comprise providing nutrients. The nutrients may be provided in a container (e.g., a first container) of the bioreactor system. The nutrients may selectively promote growth of the microbial strain and/or sustain the established population. In some embodiments, maintaining the concentration of the microbial strain may comprise applying selective pressure. The selective pressure may comprise a selective pressure described herein. In some embodiments, the selective pressure may be applied in at least one container of the bioreactor system (e.g., in at least the first container). The selective pressure may comprise conditions that reduce a growth rate of microbes in a container (e.g., in the first container). The growth rate of microbes in the container may be reduced relative to a growth rate of the microbial strain (e.g., the established population of the microbial strain).

In some embodiments, the biostimulant composition can comprise at least a portion of the established population of the microbial strain. In some embodiments, the biostimulant composition can comprise an amount of the microbial strain. The amount of the microbial strain may be a concentration of the microbial strain. The amount of the microbial strain can be at least about 1 CFU/ml, at least about 10 CFU/ml, at least about 100 CFU/ml, at least about 1×10³ CFU/ml, at least about 1×10⁴ CFU/ml, at least about 1×10⁵ CFU/ml, at least about 1×10⁶ CFU/ml, at least about 1×10⁷ CFU/ml, at least about 1×10⁸ CFU/ml, at least about 1×10⁹ CFU/ml, at least about 1×10¹⁰ CFU/ml, or greater than about 1×10¹⁰ CFU/ml. The amount of the microbial strain can be at most about 1×10¹⁰ CFU/ml, at most about 1×10⁹ CFU/ml, at most about 1×10⁸ CFU/ml, at most about 1×10⁷ CFU/ml, at most about 1×10⁶ CFU/ml, at most about 1×10⁵ CFU/ml, at most about 1×10⁴ CFU/ml, at most about 1×10³ CFU/ml, at most about 100 CFU/ml, at most about 10 CFU/ml, at most about 1 CFU/ml, or less than about 1 CFU/ml.

The established population of the microbial strain (e.g., at least a portion of the established population of the microbial strain) may be configured to promote a plant growth promoting property (e.g., increase nutrient uptake of a plant). Administering the biostimulant composition to a plant may promote a plant growth promoting property (e.g., increase a nutrient uptake) of the plant compared to that of a plant not administered the biostimulant composition.

As an example, the present disclosure provides a method, comprising: (a) providing a bioreactor system comprising two or more containers arranged in a series, each of the two or more containers comprising a volume of a working fluid, wherein a first container comprises an established population of a microbial strain having a plant growth promoting property; (b) operating the bioreactor system for a duration of time by: (i) transferring into the first container an aqueous feedstock comprising a microbial consortium; (ii) transferring a portion of the working fluid out of each of the two or more containers into either a subsequent container of the bioreactor system or a product outflow stream; and (iii) collecting at least a portion of the product outflow stream as a biostimulant composition, wherein the biostimulant composition comprises an amount of the microbial strain; wherein the amount of the microbial strain is configured to increase nutrient uptake of a plant administered the biostimulant composition as compared to a nutrient uptake of a plant not administered the biostimulant composition.

As another example, the present disclosure provides a method, comprising: (a) providing a bioreactor system comprising two or more containers arranged in a series, each of the two or more containers comprising a volume of a working fluid, wherein a first container comprises an established population of a microbial strain having a plant growth promoting property; (b) operating the bioreactor system for a duration of time by: (i) transferring into the first container an aqueous feedstock comprising a microbial consortium; (ii) transferring a portion of the working fluid out of each of the two or more containers into either a subsequent container of the bioreactor system or a product outflow stream; and (iii) collecting at least a portion of the product outflow stream as a biostimulant composition, wherein the biostimulant composition comprises an amount of the microbial strain; wherein the biostimulant composition is configured to promote a plant growth promoting property of a plant administered the biostimulant composition as compared to that of a plant not administered the biostimulant composition. The biostimulant composition can be capable of promoting a plant growth promoting property.

The nutrient uptake of the plant may be measured by an assay including, but not limited to, analysis of biomass, depletion method, or nutrient balance method. The nutrient uptake may comprise macronutrients. The macronutrients can comprise nitrogen, sulfur, phosphorous, potassium, magnesium, calcium, or any combination thereof. The nutrient uptake may comprise micronutrients. The micronutrients can comprise boron, zinc, manganese, iron, copper, or a combination thereof. In some embodiments, the nutrient uptake may comprise a combination of macronutrients and micronutrients.

A plant growth promoting property may comprise any plant growth promoting property described herein. For example a plant growth promoting property can comprise an ability to promote uptake of a nutrient by a plant, increase the bioavailability of a nutrient in soil, recruit microbes having the plant growth promoting property to the root zones or other tissues of plants, stimulate plant growth, increase yield of a crop, increase shoot biomass, increase root biomass, increase deaminase activity, increase acid production, increase leaf area, increase chlorophyll content, increase heat tolerance, increase cold tolerance, increase drought tolerance, increase salt tolerance, increase total biomass, or any combination thereof. In some embodiments, the plant growth promoting property (e.g., desired plant growth promoting property) may not comprise nitrogen use efficiency, zinc solubilization, phosphate solubilization, or any combination thereof.

A plant administered the biostimulant composition described herein may have an increased biomass compared to the plant not administered the biostimulant composition. For example, a biomass (e.g., a total biomass) of a plant administered the biostimulant composition described herein can be increased by at least about 1%, at least about 2%, at least about 3%, at least about 4%, at least about 5%, at least about 10%, at least about 15%, at least about 20%, at least about 25%, or greater than about 25% as compared to that of a plant not administered the biostimulant composition. A biomass (e.g., a total biomass) of a plant administered the biostimulant composition described herein can be increased by at most about 25%, at most about 20%, at most about 15%, at most about 10%, at most about 5%, at most about 4%, at most about 3%, at most about 2%, at most about 1%, or less than about 1% as compared to that of a plant not administered the biostimulant composition. In some embodiments, a biomass (e.g., a total biomass) of a plant administered the biostimulant composition described herein can be increased from about 1% to about 20% as compared to that of a plant not administered the biostimulant composition. In some embodiments, a biomass (e.g., a total biomass) of a plant administered the biostimulant composition described herein can be increased from about 1% to about 2%, about 1% to about 3%, about 1% to about 4%, about 1% to about 5%, about 1% to about 6%, about 1% to about 7%, about 1% to about 8%, about 1% to about 10%, about 1% to about 12%, about 1% to about 15%, about 1% to about 20%, about 2% to about 3%, about 2% to about 4%, about 2% to about 5%, about 2% to about 6%, about 2% to about 7%, about 2% to about 8%, about 2% to about 10%, about 2% to about 12%, about 2% to about 15%, about 2% to about 20%, about 3% to about 4%, about 3% to about 5%, about 3% to about 6%, about 3% to about 7%, about 3% to about 8%, about 3% to about 10%, about 3% to about 12%, about 3% to about 15%, about 3% to about 20%, about 4% to about 5%, about 4% to about 6%, about 4% to about 7%, about 4% to about 8%, about 4% to about 10%, about 4% to about 12%, about 4% to about 15%, about 4% to about 20%, about 5% to about 6%, about 5% to about 7%, about 5% to about 8%, about 5% to about 10%, about 5% to about 12%, about 5% to about 15%, about 5% to about 20%, about 6% to about 7%, about 6% to about 8%, about 6% to about 10%, about 6% to about 12%, about 6% to about 15%, about 6% to about 20%, about 7% to about 8%, about 7% to about 10%, about 7% to about 12%, about 7% to about 15%, about 7% to about 20%, about 8% to about 10%, about 8% to about 12%, about 8% to about 15%, about 8% to about 20%, about 10% to about 12%, about 10% to about 15%, about 10% to about 20%, about 12% to about 15%, about 12% to about 20%, or about 15% to about 20% as compared to that of a plant not administered the biostimulant composition.

A plant administered the biostimulant composition described herein may have an increased average leaf area compared to the plant not administered the biostimulant composition. For example, an average leaf area of a plant administered the biostimulant composition described herein can be increased by at least about 1%, at least about 2%, at least about 3%, at least about 4%, at least about 5%, at least about 10%, at least about 15%, at least about 20%, at least about 25%, at least about 30%, at least about 40%, at least about 50%, or greater than about 50% as compared to that of a plant not administered the biostimulant composition. An average leaf area of a plant administered the biostimulant composition described herein can be increased by at most about 50%, at most about 40%, at most about 30%, at most about 25%, at most about 20%, at most about 15%, at most about 10%, at most about 5%, at most about 4%, at most about 3%, at most about 2%, at most about 1%, or less than about 1% as compared to that of a plant not administered the biostimulant composition.

In some embodiments, an average leaf area of a plant administered the biostimulant composition described herein can be increased from about 1% to about 50% as compared to that of a plant not administered the biostimulant composition. In some embodiments, an average leaf area of a plant administered the biostimulant composition described herein can be increased from about 1% to about 2%, about 1% to about 3%, about 1% to about 4%, about 1% to about 5%, about 1% to about 10%, about 1% to about 15%, about 1% to about 20%, about 1% to about 25%, about 1% to about 30%, about 1% to about 40%, about 1% to about 50%, about 2% to about 3%, about 2% to about 4%, about 2% to about 5%, about 2% to about 10%, about 2% to about 15%, about 2% to about 20%, about 2% to about 25%, about 2% to about 30%, about 2% to about 40%, about 2% to about 50%, about 3% to about 4%, about 3% to about 5%, about 3% to about 10%, about 3% to about 15%, about 3% to about 20%, about 3% to about 25%, about 3% to about 30%, about 3% to about 40%, about 3% to about 50%, about 4% to about 5%, about 4% to about 10%, about 4% to about 15%, about 4% to about 20%, about 4% to about 25%, about 4% to about 30%, about 4% to about 40%, about 4% to about 50%, about 5% to about 10%, about 5% to about 15%, about 5% to about 20%, about 5% to about 25%, about 5% to about 30%, about 5% to about 40%, about 5% to about 50%, about 10% to about 15%, about 10% to about 20%, about 10% to about 25%, about 10% to about 30%, about 10% to about 40%, about 10% to about 50%, about 15% to about 20%, about 15% to about 25%, about 15% to about 30%, about 15% to about 40%, about 15% to about 50%, about 20% to about 25%, about 20% to about 30%, about 20% to about 40%, about 20% to about 50%, about 25% to about 30%, about 25% to about 40%, about 25% to about 50%, about 30% to about 40%, about 30% to about 50%, or about 40% to about 50% as compared to that of a plant not administered the biostimulant composition.

In some embodiments, the established population of the microbial strain can comprise a zinc solubilizing microbial strain. An average leaf area of a plant administered the biostimulant composition (e.g., a biostimulant comprising a zinc solubilizing microbial strain) can be increased by at least about 1%, at least about 2%, at least about 3%, at least about 4%, at least about 5%, at least about 10%, at least about 15%, at least about 20%, at least about 25%, at least about 30%, at least about 40%, at least about 50%, or greater than about 50% as compared to that of a plant not administered the biostimulant composition. An average leaf area of a plant administered the biostimulant composition (e.g., a biostimulant comprising a zinc solubilizing microbial strain) can be increased by at most about 50%, at most about 40%, at most about 30%, at most about 25%, at most about 20%, at most about 15%, at most about 10%, at most about 5%, at most about 4%, at most about 3%, at most about 2%, at most about 1%, or less than about 1% as compared to that of a plant not administered the biostimulant composition.

In some embodiments, administering the biostimulant composition (e.g., a biostimulant comprising a zinc solubilizing microbial strain) to a plant can increase a macronutrient content (e.g., a nitrogen content, a phosphorous content, a potassium content, or any combination thereof) by at least about 1 μg, at least about 2 μg, at least about 3 μg, at least about 4 μg, at least about 5 μg, at least about 10 μg, at least about 15 μg, at least about 20 μg, at least about 25 ug, at least about 30 μg, at least about 40 μg, at least about 50 μg, or greater than about 50 μg as compared to that of a plant not administered the biostimulant composition. In some embodiments, administering the biostimulant composition (e.g., a biostimulant comprising a zinc solubilizing microbial strain) to a plant can increase a macronutrient content (e.g., a nitrogen content, a phosphorous content, a potassium content, or any combination thereof) by at most about 50 μg, at most about 40 μg, at most about 30 μg, at most about 25 μg, at most about 20 μg, at most about 15 μg, at most about 10 μg, at most about 5 μg, at most about 4 μg, at most about 3 μg, at most about 2 μg, at most about 1 μg, or less than about 1 μg as compared to that of a plant not administered the biostimulant composition.

In some embodiments, the established population of the microbial strain can comprise a phosphate solubilizing microbial strain. An average leaf area of a plant administered the biostimulant composition (e.g., a biostimulant comprising a phosphate solubilizing microbial strain) can be increased by at least about 1%, at least about 2%, at least about 3%, at least about 4%, at least about 5%, at least about 10%, at least about 15%, at least about 20%, at least about 25%, at least about 30%, at least about 40%, at least about 50%, or greater than about 50% as compared to that of a plant not administered the biostimulant composition. An average leaf area of a plant administered the biostimulant composition (e.g., a biostimulant comprising a phosphate solubilizing microbial strain) can be increased by at most about 50%, at most about 40%, at most about 30%, at most about 25%, at most about 20%, at most about 15%, at most about 10%, at most about 5%, at most about 4%, at most about 3%, at most about 2%, at most about 1%, or less than about 1% as compared to that of a plant not administered the biostimulant composition.

In some embodiments, administering the biostimulant composition (e.g., a biostimulant comprising a phosphate solubilizing microbial strain) to a plant can increase a crop yield by at least about 1%, at least about 2%, at least about 3%, at least about 4%, at least about 5%, at least about 10%, at least about 15%, at least about 20%, at least about 25%, at least about 30%, at least about 40%, at least about 50%, or greater than about 50% as compared to that of a plant not administered the biostimulant composition. In some embodiments, administering the biostimulant composition (e.g., a biostimulant comprising a phosphate solubilizing microbial strain) to a plant can increase a crop yield by at most about 50%, at most about 40%, at most about 30%, at most about 25%, at most about 20%, at most about 15%, at most about 10%, at most about 5%, at most about 4%, at most about 3%, at most about 2%, at most about 1%, or less than about 1% as compared to that of a plant not administered the biostimulant composition.

In some embodiments, administering the biostimulant composition (e.g., a biostimulant comprising a phosphate solubilizing microbial strain) to a plant can increase a dry biomass by at least about 1%, at least about 2%, at least about 3%, at least about 4%, at least about 5%, at least about 10%, at least about 15%, at least about 20%, at least about 25%, at least about 30%, at least about 40%, at least about 50%, or greater than about 50% as compared to that of a plant not administered the biostimulant composition. In some embodiments, administering the biostimulant composition (e.g., a biostimulant comprising a phosphate solubilizing microbial strain) to a plant can increase a dry biomass by at most about 50%, at most about 40%, at most about 30%, at most about 25%, at most about 20%, at most about 15%, at most about 10%, at most about 5%, at most about 4%, at most about 3%, at most about 2%, at most about 1%, or less than about 1% as compared to that of a plant not administered the biostimulant composition.

In some embodiments, the established population of the microbial strain can comprise a nitrogen use efficiency-promoting microbial strain. An average leaf area of a plant administered the biostimulant composition (e.g., a biostimulant comprising a nitrogen use efficiency-promoting microbial strain) can be increased by at least about 1%, at least about 2%, at least about 3%, at least about 4%, at least about 5%, at least about 10%, at least about 15%, at least about 20%, at least about 25%, at least about 30%, at least about 40%, at least about 50%, at least about 60%, at least about 70%, at least about 80%, or greater than about 80% as compared to that of a plant not administered the biostimulant composition. An average leaf area of a plant administered the biostimulant composition (e.g., a biostimulant comprising a nitrogen use efficiency-promoting microbial strain) can be increased by at most about 80%, at most about 70%, at most about 60%, at most about 50%, at most about 40%, at most about 30%, at most about 25%, at most about 20%, at most about 15%, at most about 10%, at most about 5%, at most about 4%, at most about 3%, at most about 2%, at most about 1%, or less than about 1% as compared to that of a plant not administered the biostimulant composition.

In some embodiments, administering the biostimulant composition (e.g., a biostimulant comprising a nitrogen use efficiency-promoting microbial strain) to a plant can increase a dry shoot weight, a dry root weight, a stem diameter, or any combination thereof by at least about 1%, at least about 2%, at least about 3%, at least about 4%, at least about 5%, at least about 10%, at least about 15%, at least about 20%, at least about 25%, at least about 30%, at least about 40%, at least about 50%, or greater than about 50% as compared to that of a plant not administered the biostimulant composition. In some embodiments, administering the biostimulant composition (e.g., a biostimulant comprising a nitrogen use efficiency-promoting microbial strain) to a plant can increase a dry shoot weight, a dry root weight, a stem diameter, or any combination thereof by at most about 50%, at most about 40%, at most about 30%, at most about 25%, at most about 20%, at most about 15%, at most about 10%, at most about 5%, at most about 4%, at most about 3%, at most about 2%, at most about 1%, or less than about 1% as compared to that of a plant not administered the biostimulant composition.

In some embodiments, administering the biostimulant composition (e.g., a biostimulant comprising a nitrogen use efficiency-promoting microbial strain) to a plant can increase a leaf chlorophyll content by at least about 1%, at least about 2%, at least about 3%, at least about 4%, at least about 5%, at least about 10%, at least about 15%, at least about 20%, at least about 25%, at least about 30%, at least about 40%, at least about 50%, or greater than about 50% as compared to that of a plant not administered the biostimulant composition. In some embodiments, administering the biostimulant composition (e.g., a biostimulant comprising a nitrogen use efficiency-promoting microbial strain) to a plant can increase a leaf chlorophyll content by at most about 50%, at most about 40%, at most about 30%, at most about 25%, at most about 20%, at most about 15%, at most about 10%, at most about 5%, at most about 4%, at most about 3%, at most about 2%, at most about 1%, or less than about 1% as compared to that of a plant not administered the biostimulant composition.

In some embodiments, prior to (a) of a method described herein, an inoculum of the microbial strain may be added to the bioreactor system (e.g., may be added to a container of the bioreactor system). An inoculum of the microbial strain may be added to one or more containers of the bioreactor system (e.g., a first container, a second container, a third container, a fourth container, a fifth container, or any combination thereof). Adding the inoculum of the microbial strain may produce an initial population of the microbial strain. The initial population of the microbial strain may be present at a concentration in at least one container of the bioreactor system. For example, an initial population of the microbial strain may be present at at least about 10 CFU/ml, at least about 100 CFU/ml, at least about 0.5×10³ CFU/ml, at least about 1×10³ CFU/ml, at least about 0.5×10⁴ CFU/ml, at least about 1×10⁴ CFU/ml, at least about 0.5×10⁵ CFU/ml, at least about 1×10⁵ CFU/ml, at least about 1×10⁶ CFU/ml, or greater than about 1×10⁶ CFU/ml in at least one container. An initial population of the microbial strain may be present at at most about 1×10⁶ CFU/ml, at most about 1×10⁵ CFU/ml, at most about 0.5×10⁵ CFU/ml, at most about 1×10⁴ CFU/ml, at most about 0.5×10⁴ CFU/ml, at most about 1×10³ CFU/ml, at most about 0.5×10³ CFU/ml, at most about 100 CFU/ml, at most about 10 CFU/ml, or less than about 10 CFU/ml.

The aqueous feedstock may comprise an organic material. The organic material can be at least partially digestible by microbes (e.g., microbes present in at least one container of the bioreactor system). In some embodiments, the organic material may have been partially digested by microbes endogenous to the organic material. The organic material may have been partially digested prior to a transferring of step (b) (i) of a method described herein. In some embodiments, a method described herein may further comprise digesting the organic material in one or more containers. In some embodiments, a method described herein may further comprise digesting the organic material in serially connected containers (e.g., two or more serially connected containers). The digesting of the organic material can occur before the transferring of step (b) (i) of a method described herein. The organic material may comprise an organic material described herein.

The aqueous feedstock may comprise an inorganic material. The inorganic material can comprise rock phosphate (e.g., rock phosphate particles). In some embodiments, the inorganic material (e.g., rock phosphate particles) may have been partially digested by microbes (e.g., microbes present in the aqueous feedstock). The inorganic material may have been partially digested by microbes (e.g., microbes present in the aqueous feedstock). In some embodiments, a method described herein may further comprise digesting the inorganic material (e.g., rock phosphate particles) in one or more containers. In some embodiments, a method described herein may further comprise digesting the inorganic material (e.g., rock phosphate particles) in serially connected containers (e.g., two or more serially connected containers). The digesting of the inorganic material (e.g., rock phosphate particles) can occur before the transferring of step (b) (i) of a method described herein. In some embodiments, digestion (e.g., partial digestion) of the organic material and inorganic material can occur at the same time. In some embodiments, digestion (e.g., partial digestion) of the organic material and inorganic material can occur at different times. In some embodiments, digestion (e.g., partial digestion) of the organic material and inorganic material can occur in the same container (e.g., a same container of the bioreactor system). For example, the organic material and inorganic material may be digested or partially digested in a first container. In some embodiments, digestion (e.g., partial digestion) of the organic material and inorganic material can occur in different containers (e.g., different containers of the bioreactor system). For example, the organic material may be digested or partially digested in a first container and the inorganic material may be digested or partially digested in a second container.

The microbial consortium of the aqueous feedstock may be present at a concentration. In some embodiments, the microbial consortium can comprise at least about 100 CFU/ml, at least about 1×10³ CFU/ml, at least about 1×10⁴ CFU/ml, at least about 1×10⁵ CFU/ml, at least about 1×10⁶ CFU/ml, at least about 1×10⁷ CFU/ml, at least about 1×10⁸ CFU/ml, at least about 1×10⁹ CFU/ml, at least about 1×10¹⁰ CFU/ml, or greater than about 1×10¹⁰ CFU/ml. In some embodiments, the microbial consortium can comprise at most about 1×10¹⁰ CFU/ml, at most about 1×10⁹ CFU/ml, at most about 1×10⁸ CFU/ml, at most about 1×10⁷ CFU/ml, at most about 1×10⁶ CFU/ml, at most about 1×10⁵ CFU/ml, at most about 1×10⁴ CFU/ml, at most about 1×10³ CFU/ml, at most about 100 CFU/ml, or less than about 100 CFU/ml. The microbial consortium may comprise microbes derived from an organic material, an inorganic material, or any combination thereof. In some embodiments, the microbial consortium may comprise microbes derived manure, rock phosphate particles, or any combination thereof.

In some embodiments, a method described herein may further comprise producing metabolites. The metabolites may be produced from microbes of the bioreactor system. For example, metabolites may be produced from the microbial consortium, the population of the microbial strain (e.g., the established population of the microbial strain), or any combination thereof. In some embodiments, the metabolites (e.g., microbial metabolites) may have a plant growth promoting property. The metabolites (e.g., microbial metabolites) may have two or more plant growth promoting properties. In some embodiments, the metabolites (e.g., microbial metabolites) may have the same plant growth promoting property as the established population of the microbial strain. For example, the metabolites (e.g., microbial metabolites) and the established population of the microbial strain may increase nutrient uptake of a plant.

In some embodiments, the transferring of step (b) (i), the transferring of step (b) (ii), the collecting of step (b), or any combination thereof of the method described herein may be performed continuously. The transferring and/or collecting of the method described herein can be performed continuously over the duration of time. In some embodiments, the transferring of step (b) (i), the transferring of step (b) (ii), the collecting of step (b), or any combination thereof of the method described herein may be performed periodically. The transferring and/or collecting of the method described herein can be performed periodically over the duration of time.

A method described herein may further comprise adding one or more carbon sources to a container (e.g., at least one container of the bioreactor system). The carbon source may comprise any carbon sources described herein. In some embodiments, a carbon source (e.g., one or more carbon sources) may be comprised in the aqueous feedstock.

A bioreactor system described herein can comprise a clarifier (e.g., a clarifier container). The clarifier (e.g., a clarifier container) may comprise a working fluid (e.g., a clarifier working fluid). A method described herein can further comprise separating portions of the clarifier working fluid. In some embodiments, a method described herein comprises separating a supernatant portion of the clarifier working fluid from a floc portion of the clarifier working fluid. The separating can occur within the clarifier container. A method described herein may further comprise folding the floc portion of the clarifier working fluid. The folding may comprise releasing a population of the microbial strain. The population of the microbial strain may release into the supernatant portion (e.g., the supernatant portion of the clarifier working fluid). While the folding can release the population of the microbial strain into a supernatant portion of the clarifier working fluid, the folding may not introduce solid from the floc portion (e.g., floc solids) into the supernatant portion of the clarifier working fluid.

In some embodiments, the method can further comprise transferring a floc portion. The floc portion may be transferred from the clarifier container to a container of bioreactor system. The floc portion may be reintroduced into an earlier container in the bioreactor system by transferring the floc portion from the clarifier container to the earlier container. For example, the floc portion may be transferred from the clarifier container to a first container, second container, third container, fourth container, fifth container, sixth container, or any combination thereof of the bioreactor system. The product outflow stream of the bioreactor system can comprise the supernatant portion of the clarifier working fluid. The product outflow stream may be collected from the bioreactor system.

In some embodiments, a method of making a biostimulant composition is performed using a bioreactor system that comprises two or more containers arranged in a series. A series of containers can comprise 2, 3, 4, 5, 6, 7 or more containers. The containers may be the same or different. A series of containers may be fluidly connected to the other containers in the series. A series of containers may be arranged such that fluid can flow from a first container to a second container and from a second container to a third container, and so on from each subsequent container to a next container in the series. Referring to a container as a “first container” does not necessarily indicate that the referenced container is the most upstream container in a series of container. In some embodiments, additional containers in a series are upstream of a container referred to as a “first container.” In some embodiments, a container that is the most upstream container in a series of fluidly connected containers does not have another container upstream of the container. In some embodiments, a container that is the most upstream container in a series receives an inflow stream such as, for example, a feedstock stream. In some embodiments, a feedstock stream comes from a mixing container, which may be referred to as a mixing tank or complete mix reactor. In some embodiments, components that are placed in a mixing container have a shorter dwell time than working fluids in subsequent containers in the system. In some embodiments, a container that is the most downstream container in a series is a clarifier, which may be configured to separate a supernatant portion of a working fluid from a floc portion of the working fluid. The most downstream container may not be a clarifier and may not be configured to separate different portions of a working fluid. In some embodiments, the most downstream container has an outlet port through which a product outflow stream may exit the most downstream container. Containers arranged in a series may have inflows other than those coming from the neighboring upstream container and may have outflows other than those coming from the neighboring downstream container.

In some embodiments, a reactor or a series of reactors (e.g., serialized assembly of reactors) functions to contribute to the growth of one or more microbes having desired plant growth promoting properties and/or to the production of digestion products having plant growth promoting properties. In some embodiments, the operation of a bioreactor system may lead to growth of one or more microbes having a desired plant growth promoting effect. The one or more microbes may be one or more isolated microbes added separately as an inoculum to the bioreactor system. The one or more microbes may also be input into the system as part of a feed material that comprises a mixture of microbes. The one or more microbes may be endogenous to an organic material such as, for example, a manure, a plant, a lignocellulosic material, or an algae. The one or more microbes may also be endogenous to other types of feed materials, such as rock phosphate. In some embodiments, endogenous microbes are those microbes naturally present in feedstock material (e.g., a manure, a plant, a lignocellulosic material, or an algae). These microbes may naturally reside in a closed system and/or are present in the ecosystem of the feedstock material.

In some embodiments, the bioreactor system may be operated in a hydraulically balanced manner. A hydraulically balanced manner can comprise working fluid transferring from one container of the system to another container to a further container at an equal rate. In some embodiments, the transferring and/or collecting of the methods described herein may be driven by gravity. The outlet port of a container may be higher than an inlet port of another container so that the working fluid transfers via gravity between containers of the bioreactor system. In some embodiments, the opening of an outlet port of a first container is at the same level as the top of the working fluid or slightly below the level of the working fluid (i.e., the bottom of the opening of the outlet port may be 1, 2, 3, or 4 cm below the level of the working fluid) so that the rate of flow of liquid into the first container can be equaled by the rate of flow of liquid out of the first container. The first container may be fluidly connected to a second container, e.g., by a pipe or other conduit connected to the outlet port of the first container. The pipe or other conduit may connect to an inlet port of a second container. The inlet port of the second container may be below the level of the outlet port of the first container such that the working fluid from the first container may flow by gravity alone into the second container. The second container may have an outlet port with an opening that is at the same level or is at least partially below the level of the working fluid in the second container (i.e., the bottom of the opening of the outlet port may be 1, 2, 3, or 4 cm below the level of the working fluid) so that the rate of flow of liquid into the first container can be equaled by the rate of flow of liquid out of the first container solely due to the effect of gravity. Additional containers (e.g., a third, fourth, fifth, sixth, seventh, or more containers) can have inlet and outlet ports arranged in the same way, with each subsequent container having inlet and outlet ports arranged such that flow between containers can be continuous and driven solely by gravity, i.e., the inlet and outlet ports of each subsequent container in a series of containers is at a level lower than the outlet port of the previous container. In some embodiments, all the inlet and outlet ports of containers in the system are open at all times to allow continuous flow driven solely by gravity (i.e., without any pumps needed to drive flow from any container to a subsequent container in the system). Operating the bioreactor system can comprise maintaining a flow rate. In some embodiments, a flow rate may be maintained to result in a hydraulic retention time of at least about 1 day, at least about 2 days, at least about 3 days, at least about 4 days, at least about 5 days, at least about 6 days, at least about 7 days, at least about 8 days, at least about 9 days, at least about 10 days, at least about 15 days, at least about 20 days, at least about 25 days, at least about 30 days, or greater than about 30 days. In some embodiments, a flow rate may be maintained to result in a hydraulic retention time of at most about 30 days, at most about 25 days, at most about 20 days, at most about 15 days, at most about 10 days, at most about 9 days, at most about 8 days, at most about 7 days, at most about 6 days, at most about 5 days, at most about 4 days, at most about 3 days, at most about 2 days, at most about 1 day, or less than about 1 day.

Operating the bioreactor system can comprise maintaining the product outflow stream. The product outflow stream may be maintained at a flow rate of at least about 5 gallons per day, at least about 10 gallons per day, at least about 25 gallons per day, at least about 50 gallons per day, at least about 75 gallons per day, at least about 100 gallons per day, at least about 150 gallons per day, at least about 250 gallons per day, at least about 500 gallons per day, at least about 1,000 gallons per day, or greater than about 1,000 gallons per day. The product outflow stream may be maintained at a flow rate of at most about 1,000 gallons per day, at most about 500 gallons per day, at most about 250 gallons per day, at most about 150 gallons per day, at most about 100 gallons per day, at most about 75 gallons per day, at most about 50 gallons per day, at most about 25 gallons per day, at most about 10 gallons per day, at most about 5 gallons per day, or less than about 5 gallons per day.

The bioreactor system may operate continuously for a duration of time. In some embodiments, the bioreactor system may be operated continuously for at least about 5 days, at least about 10 days, at least about 20 days, at least about 30 days, at least about 40 days, at least about 50 days, at least about 60 days, at least about 70 days, at least about 80 days, at least about 90 days, at least about 100 days, at least about 150 days, at least about 200 days, or greater than about 200 days. In some embodiments, the bioreactor system may be operated continuously for at most about 200 days, at most about 150 days, at most about 100 days, at most about 90 days, at most about 80 days, at most about 70 days, at most about 60 days, at most about 50 days, at most about 40 days, at most about 30 days, at most about 20 days, at most about 10 days, at most about 5 days, or less than about 5 days.

In some embodiments, the methods described herein further comprise producing bacteria. The bacteria can be sporulated bacteria and the sporulated bacteria may be in the product outflow stream. The methods may further comprise producing a population of the microbial strain (e.g., an amount of the microbial strain) in the product outflow stream. The population of the microbial strain in the product outflow stream may be sporulated. An amount (e.g., a concentration) of the population of the microbial strain in the product outflow stream may be sporulated. In some embodiments, a population of the microbial strain that is sporulated may comprise at least about 100 CFU/ml, at least about 1×10³ CFU/ml, at least about 1×10⁴ CFU/ml, at least about 1×10⁵ CFU/ml, at least about 1×10⁶ CFU/ml, at least about 1×10⁷ CFU/ml, at least about 1×10⁸ CFU/ml, at least about 1×10⁹ CFU/ml, at least about 1×10¹⁰ CFU/ml, or greater than about 1×10¹⁰ CFU/ml. In some embodiments, a population of the microbial strain that is sporulated may comprise at most about 1×10¹⁰ CFU/ml, at most about 1×10⁹ CFU/ml, at most about 1×10⁸ CFU/ml, at most about 1×10⁷ CFU/ml, at most about 1×10⁶ CFU/ml, at most about 1×10⁵ CFU/ml, at most about 1×10⁴ CFU/ml, at most about 1×10³ CFU/ml, at most about 100 CFU/ml, or less than about 100 CFU/ml. In some embodiments, the methods described herein can further comprise adding an additional population of the microbial strain to the biostimulant composition (e.g., the biostimulant product).

In an exemplary system of the present disclosure, a series of reactors functions to contribute to the growth of an inoculum of a microbe (e.g., isolate) having desired plant growth promoting properties. A series of reactors (e.g., serialized assembly of reactors) may also function to contribute to the production of microbial metabolites having desired plant growth promoting properties. The bioreactor system described herein can enrich an inoculum of a target microbe, a population of microbes within a microbial consortium with plant-growth promotion properties (e.g., zinc solubilization, phosphate solubilization, nitrogen use efficiency), metabolites produced by the enriched population of the microbial consortium with plant-growth promotion properties (e.g., zinc solubilization, phosphate solubilization, nitrogen use efficiency), or any combination thereof. This system provides added benefits to other bioreactor systems in that it can target a functional community of microbes and/or metabolites with specific functionality and enrich and/or maintain the community in the bioreactor system. The system can comprise two reactor chambers, three reactor chambers, four reactor chambers, five reactor chambers, or more reactor chambers (e.g., containers or chambers). Without wishing to be bound by theory, the serialized reactors enable the growth and enrichment of proprietary specialist target microbes with optimal plant growth promoting properties. The system may direct a flow of working fluid comprising an inoculum, carbon source, and/or nutrient source from an input feedstock (e.g., organic feedstock) to produce a base product (BP). A hydraulic source can flow into a reactor via in-flow port to comprise a first working fluid in a reactor tank. A substance providing selective pressure can flow into a reactor via in-flow port to comprise a first working fluid in a reactor tank. A hydraulic source and a substance providing selective pressure can flow into a reactor via in-flow port to comprise a first working fluid in a reactor tank. A hydraulic source may input into a first reactor or any reactor of the system. In some embodiments, a hydraulic source may input (e.g., flow) into a tank or container prior to a first reactor. In some embodiments, the container may comprise a “complete mixed reactor” (CMR). Other inputs into a system described herein may flow into any reactor of the system, including but not limited to a first reactor, a second reactor, a third reactor, or any other reactor following a first reactor. In some embodiments, the bioreactor system can comprise at least one container prior to a first container (e.g., at least one container in the series of container before the first container).

In some embodiments, a method of making a biostimulant composition is described herein as including operating a bioreactor system for a duration of time. It is contemplated that the bioreactor system may be operated both before and after the duration of time in the same way as during the duration of time. Some embodiments of methods described herein may describe steps taking place during a particular duration of time, such as, for example, 1 day, 2 days, 3 days, 4 days, 5 days, 10 days, 15 days, 30 days, 60 days, 90 days, 120 days, 5 months, 6 months, 7 months, 8 months, 9 months, 10 months, 11 months, 1 year, 2 years, 3 years, 4 years, 5 years or more. Those durations of time described herein may be a fraction of a larger duration of time during which the bioreactor system is operating continuously or mostly continuously.

A digestion process to produce the biostimulant may be performed in a bioreactor system that comprises a tank, a container, a vessel (e.g., reactor), or a series of tanks, a series of containers, or a series of vessels (e.g., reactors) through which the feedstock continuously flows. A reactor can be a fluidly connected container, system, vessel, or tank in which microbial consortia including the microbes, isolates, and/or microorganisms as described herein can be grown. A reactor can be separate or continuous. A reactor may be a physical containment system arranged in a discrete order to favor growth of particular microbes. Types of reactors can include, but are not limited to, fluidized-bed reactors (FBRs) or packed-bed reactors (PBRs).

In an aspect, the present disclosure provides a bioreactor system. The bioreactor system can comprise a stream of an aqueous feedstock. The stream may be in fluid communication with a container (e.g., a first container). The container can comprise a volume of working fluid. For example, the stream may be in fluid communication with a first container of the bioreactor system comprising a volume of a first working fluid. The aqueous feedstock can comprise a microbial consortium, wherein the microbial consortium can comprise a microbial consortium described herein. The working fluid (e.g., the first working fluid) may comprise a microbial strain. The microbial strain can be a population of a microbial strain and may comprise a plant growth promoting property (e.g., a desired plant growth promoting property). A first container of the bioreactor system may comprise a mixer. The mixer can be configured to aerate the working fluid (e.g., the first working fluid) in a container.

In some embodiments, a concentration of the microbial strain may be higher in the working fluid than in the aqueous feedstock. In some embodiments, a concentration of the microbial strain may be higher in the working fluid than in any other input to the bioreactor. For example, a concentration of the microbial strain in the first working fluid may be at least about 2 times higher, at least about 5 times higher, at least about 10 times higher, at least about 25 times higher, at least about 50 times higher, at least about 100 times higher, at least about 150 times higher, at least about 200 times higher, at least about 250 times higher, at least about 500 times higher, or greater than about 500 times higher than a concentration of the microbial strain in the aqueous feedstock stream or in any other input into the bioreactor system. A concentration of the microbial strain in the first working fluid may be at most about 500 times higher, at most about 250 times higher, at most about 200 times higher, at most about 150 times higher, at most about 100 times higher, at most about 50 times higher, at most about 25 times higher, at most about 10 times higher, at most about 5 times higher, at most about 2 times higher, or less than about 2 times higher than a concentration of the microbial strain in the aqueous feedstock stream or in any other input into the bioreactor system.

The bioreactor system described herein may comprise one or more additional containers (e.g., one or more containers in addition to a first container). The container can be arranged in a series. In some embodiments, the series of containers can comprise a first container, a second container, a third container, a fourth container, a fifth container, a sixth container, and any additional container(s). One or more additional containers in the bioreactor system may comprise a volume of working fluid. In some embodiments, each container of the one or more additional containers comprises a volume of working fluid. The one or more additional containers may be in fluid communication with a container of the series in the bioreactor system. In some embodiments, at least one of the containers of the bioreactor system can comprise a product outflow stream port. In some embodiments, each container of the series of containers may comprise a product outflow stream port. A product outflow stream (e.g., stream of the volume of working fluid of a container) can be in fluid communication with the product outflow stream port.

As an example, the present disclosure provides a bioreactor system comprising: (a) a stream of an aqueous feedstock in fluid communication with a first container comprising a volume of a first working fluid, wherein the aqueous feedstock comprises a microbial consortium, wherein the first working fluid comprises a population of a microbial strain having a desired plant growth promoting property, wherein a concentration of the microbial strain in the first working fluid is at least 100 times higher than a concentration of the microbial strain in the aqueous feedstock stream or in any other input into the bioreactor system; (b) one or more additional containers arranged in a series that comprises the first container, wherein each of the one or more additional containers comprises a volume of a working fluid and is in fluid communication with at least one other container in the series, and wherein at least one of the one or more additional containers comprises a product outflow stream port; and (c) a product outflow stream in fluid communication with the product outflow stream port.

In some embodiments, the product outflow stream may comprise a biostimulant composition. The biostimulant composition can comprise an amount of the microbial strain, wherein the amount of the microbial strain can be an amount described herein. The biostimulant composition can comprise a plant growth promoting property (e.g., a desired plant growth promoting property). In some embodiments, the amount of microbial strain can be configured to promote a plant growth promoting property (e.g., an ability to promote uptake of a nutrient by a plant, increase the bioavailability of a nutrient in soil, recruit microbes having the plant growth promoting property to the root zones or other tissues of plants, stimulate plant growth, increase yield of a crop, increase shoot biomass, increase root biomass, increase deaminase activity, increase acid production, increase leaf area, increase chlorophyll content, increase heat tolerance, increase cold tolerance, increase drought tolerance, increase salt tolerance, increase total biomass, or any combination thereof).

As another example, a bioreactor system can comprise: (a) a stream of an aqueous feedstock in fluid communication with a first container comprising a volume of a first working fluid, wherein the aqueous feedstock comprises a microbial consortium, wherein the first working fluid comprises a population of a microbial strain having a desired plant growth promoting property; (b) one or more additional containers arranged in a series that comprises the first container, wherein each of the one or more additional containers comprises a volume of a working fluid and is in fluid communication with at least one other container in the series, and wherein at least one of the one or more additional containers comprises a product outflow stream port; and (c) a product outflow stream in fluid communication with the product outflow stream port, wherein the product outflow stream comprises a biostimulant composition, wherein the biostimulant composition comprises an amount of the microbial strain, wherein the amount of the microbial strain is configured to promote a plant growth promoting property of a plant administered the biostimulant composition as compared to the plant not administered the biostimulant composition. The microbial strain and/or the biostimulant composition described herein can be capable of promoting a plant growth promoting property.

As another example, a bioreactor system can comprise: (a) a stream of an aqueous feedstock in fluid communication with a first container comprising a volume of a first working fluid, wherein the aqueous feedstock comprises a microbial consortium, wherein the first working fluid comprises a population of a microbial strain having a desired plant growth promoting property; (b) one or more additional containers arranged in a series that comprises the first container, wherein each of the one or more additional containers comprises a volume of a working fluid and is in fluid communication with at least one other container in the series, and wherein at least one of the one or more additional containers comprises a product outflow stream port; and (c) a product outflow stream in fluid communication with the product outflow stream port, wherein the product outflow stream comprises a biostimulant composition, wherein the biostimulant composition comprises an amount of the microbial strain, wherein the amount of the microbial strain at least a portion of the population of the microbial strain is configured to increase nutrient uptake of a plant administered the biostimulant composition as compared to a nutrient uptake of the plant not administered the biostimulant composition. In some embodiments, the microbial strain and/or the biostimulant composition described herein may be capable of increasing nutrient uptake of a plant.

The bioreactor system can be a continuous flow system. A continuous flow system can comprise a system wherein the flow of fluid is not interrupted. The stream of aqueous feedstock can be a continuous stream. In some embodiments, the bioreactor system can comprise a periodic flow, wherein there can be interruptions to the flow stream. In some embodiments, as a continuous flow bioreactor system, the volume of working fluid may be constant.

In some embodiments, the bioreactor system can comprise the clarifier container as described herein. The clarifier container can be configured to separate a portion of a working fluid in the clarifier container into a supernatant portion and a floc portion. In some embodiments, the product outflow stream of the system can comprise the supernatant portion.

In some embodiments, the product outflow stream comprises at least about 100 CFU/ml, at least about 1×10³ CFU/ml, at least about 1×10⁴ CFU/ml, at least about 1×10⁵ CFU/ml, at least about 1×10⁶ CFU/ml, at least about 1×10⁷ CFU/ml, at least about 1×10⁸ CFU/ml, at least about 1×10⁹ CFU/ml, at least about 1×10¹⁰ CFU/ml, or greater than about 1×10¹⁰ CFU/ml of the microbial strain. In some embodiments, the product outflow stream comprises at most about 1×10¹⁰ CFU/ml, at most about 1×10⁹ CFU/ml, at most about 1×10⁸ CFU/ml, at most about 1×10⁷ CFU/ml, at most about 1×10⁶ CFU/ml, at most about 1×10⁵ CFU/ml, at most about 1×10⁴ CFU/ml, at most about 1×10³ CFU/ml, at most about 100 CFU/ml, or less than about 100 CFU/ml of the microbial strain.

In some embodiments, the product outflow stream comprises at least about 100 CFU/ml, at least about 1×10³ CFU/ml, at least about 1×10⁴ CFU/ml, at least about 1×10⁵ CFU/ml, at least about 1×10⁶ CFU/ml, at least about 1×10⁷ CFU/ml, at least about 1×10⁸ CFU/ml, at least about 1×10⁹ CFU/ml, at least about 1×10¹⁰ CFU/ml, or greater than about 1×10¹⁰ CFU/ml of a sporulated form of the microbial strain. In some embodiments, the product outflow stream comprises at most about 1×10¹⁰ CFU/ml, at most about 1×10⁹ CFU/ml, at most about 1×10⁸ CFU/ml, at most about 1×10⁷ CFU/ml, at most about 1×10⁶ CFU/ml, at most about 1×10⁵ CFU/ml, at most about 1×10⁴ CFU/ml, at most about 1×10³ CFU/ml, at most about 100 CFU/ml, or less than about 100 CFU/ml of a sporulated form of the microbial strain.

In some embodiments, the product outflow stream may comprise a total dry weight. The total dry weight of the product outflow stream may be at least about 0.05 mg/ml, at least about 0.1 mg/ml, at least about 0.2 mg/ml, at least about 0.3 mg/ml, at least about 0.4 mg/ml, at least about 0.5 mg/ml, at least about 1.0 mg/ml, at least about 1.5 mg/ml, at least about 2.0 mg/ml, at least about 2.5 mg/ml, at least about 3.0 mg/ml, at least about 3.5 mg/ml, at least about 4.0 mg/ml, at least about 5.0 mg/ml, or greater than about 5.0 mg/ml. The total dry weight of the product outflow stream may be at most about 5.0 mg/ml, at most about 4.0 mg/ml, at most about 3.5 mg/ml, at most about 3.0 mg/ml, at most about 2.5 mg/ml, at most about 2.0 mg/ml, at most about 1.5 mg/ml, at most about 1.0 mg/ml, at most about 0.5 mg/ml, at most about 0.4 mg/ml, at most about 0.3 mg/ml, at most about 0.2 mg/ml, at most about 0.1 mg/ml, at most about 0.05 mg/ml, or less than about 0.05 mg/ml. The total dry weight of the product outflow stream may be between about 0.05 mg/ml to about 4 mg/ml. The total dry weight of the product outflow stream may be between about 0.05 mg/ml to about 0.1 mg/ml, about 0.05 mg/ml to about 0.2 mg/ml, about 0.05 mg/ml to about 0.3 mg/ml, about 0.05 mg/ml to about 0.4 mg/ml, about 0.05 mg/ml to about 0.5 mg/ml, about 0.05 mg/ml to about 1 mg/ml, about 0.05 mg/ml to about 1.5 mg/ml, about 0.05 mg/ml to about 2 mg/ml, about 0.05 mg/ml to about 2.5 mg/ml, about 0.05 mg/ml to about 3 mg/ml, about 0.05 mg/ml to about 4 mg/ml, about 0.1 mg/ml to about 0.2 mg/ml, about 0.1 mg/ml to about 0.3 mg/ml, about 0.1 mg/ml to about 0.4 mg/ml, about 0.1 mg/ml to about 0.5 mg/ml, about 0.1 mg/ml to about 1 mg/ml, about 0.1 mg/ml to about 1.5 mg/ml, about 0.1 mg/ml to about 2 mg/ml, about 0.1 mg/ml to about 2.5 mg/ml, about 0.1 mg/ml to about 3 mg/ml, about 0.1 mg/ml to about 4 mg/ml, about 0.2 mg/ml to about 0.3 mg/ml, about 0.2 mg/ml to about 0.4 mg/ml, about 0.2 mg/ml to about 0.5 mg/ml, about 0.2 mg/ml to about 1 mg/ml, about 0.2 mg/ml to about 1.5 mg/ml, about 0.2 mg/ml to about 2 mg/ml, about 0.2 mg/ml to about 2.5 mg/ml, about 0.2 mg/ml to about 3 mg/ml, about 0.2 mg/ml to about 4 mg/ml, about 0.3 mg/ml to about 0.4 mg/ml, about 0.3 mg/ml to about 0.5 mg/ml, about 0.3 mg/ml to about 1 mg/ml, about 0.3 mg/ml to about 1.5 mg/ml, about 0.3 mg/ml to about 2 mg/ml, about 0.3 mg/ml to about 2.5 mg/ml, about 0.3 mg/ml to about 3 mg/ml, about 0.3 mg/ml to about 4 mg/ml, about 0.4 mg/ml to about 0.5 mg/ml, about 0.4 mg/ml to about 1 mg/ml, about 0.4 mg/ml to about 1.5 mg/ml, about 0.4 mg/ml to about 2 mg/ml, about 0.4 mg/ml to about 2.5 mg/ml, about 0.4 mg/ml to about 3 mg/ml, about 0.4 mg/ml to about 4 mg/ml, about 0.5 mg/ml to about 1 mg/ml, about 0.5 mg/ml to about 1.5 mg/ml, about 0.5 mg/ml to about 2 mg/ml, about 0.5 mg/ml to about 2.5 mg/ml, about 0.5 mg/ml to about 3 mg/ml, about 0.5 mg/ml to about 4 mg/ml, about 1 mg/ml to about 1.5 mg/ml, about 1 mg/ml to about 2 mg/ml, about 1 mg/ml to about 2.5 mg/ml, about 1 mg/ml to about 3 mg/ml, about 1 mg/ml to about 4 mg/ml, about 1.5 mg/ml to about 2 mg/ml, about 1.5 mg/ml to about 2.5 mg/ml, about 1.5 mg/ml to about 3 mg/ml, about 1.5 mg/ml to about 4 mg/ml, about 2 mg/ml to about 2.5 mg/ml, about 2 mg/ml to about 3 mg/ml, about 2 mg/ml to about 4 mg/ml, about 2.5 mg/ml to about 3 mg/ml, about 2.5 mg/ml to about 4 mg/ml, or about 3 mg/ml to about 4 mg/ml.

In some embodiments, the product outflow stream may comprise a chemical oxygen demand. The chemical oxygen demand of the product outflow stream may be at least about 10 mg/L, at least about 20 mg/L, at least about 30 mg/L, at least about 40 mg/L, at least about 50 mg/L, at least about 60 mg/L, at least about 70 mg/L, at least about 80 mg/L, at least about 90 mg/L, at least about 100 mg/L, at least about 250 mg/L, at least about 500 mg/L, at least about 750 mg/L, at least about 1000 mg/L, or greater than about 1000 mg/L. The chemical oxygen demand of the product outflow stream may be at most about 1000 mg/L, at most about 750 mg/L, at most about 500 mg/L, at most about 250 mg/L, at most about 100 mg/L, at most about 90 mg/L, at most about 80 mg/L, at most about 70 mg/L, at most about 60 mg/L, at most about 50 mg/L, at most about 40 mg/L, at most about 30 mg/L, at most about 20 mg/L, at most about 10 mg/L, or less than about 10 mg/L. The chemical oxygen demand of the product outflow stream may be between about 10 mg/L to about 1,000 mg/L. The chemical oxygen demand of the product outflow stream may be between about 10 mg/L to about 20 mg/L, about 10 mg/L to about 30 mg/L, about 10 mg/L to about 40 mg/L, about 10 mg/L to about 50 mg/L, about 10 mg/L to about 80 mg/L, about 10 mg/L to about 100 mg/L, about 10 mg/L to about 150 mg/L, about 10 mg/L to about 200 mg/L, about 10 mg/L to about 500 mg/L, about 10 mg/L to about 750 mg/L, about 10 mg/L to about 1,000 mg/L, about 20 mg/L to about 30 mg/L, about 20 mg/L to about 40 mg/L, about 20 mg/L to about 50 mg/L, about 20 mg/L to about 80 mg/L, about 20 mg/L to about 100 mg/L, about 20 mg/L to about 150 mg/L, about 20 mg/L to about 200 mg/L, about 20 mg/L to about 500 mg/L, about 20 mg/L to about 750 mg/L, about 20 mg/L to about 1,000 mg/L, about 30 mg/L to about 40 mg/L, about 30 mg/L to about 50 mg/L, about 30 mg/L to about 80 mg/L, about 30 mg/L to about 100 mg/L, about 30 mg/L to about 150 mg/L, about 30 mg/L to about 200 mg/L, about 30 mg/L to about 500 mg/L, about 30 mg/L to about 750 mg/L, about 30 mg/L to about 1,000 mg/L, about 40 mg/L to about 50 mg/L, about 40 mg/L to about 80 mg/L, about 40 mg/L to about 100 mg/L, about 40 mg/L to about 150 mg/L, about 40 mg/L to about 200 mg/L, about 40 mg/L to about 500 mg/L, about 40 mg/L to about 750 mg/L, about 40 mg/L to about 1,000 mg/L, about 50 mg/L to about 80 mg/L, about 50 mg/L to about 100 mg/L, about 50 mg/L to about 150 mg/L, about 50 mg/L to about 200 mg/L, about 50 mg/L to about 500 mg/L, about 50 mg/L to about 750 mg/L, about 50 mg/L to about 1,000 mg/L, about 80 mg/L to about 100 mg/L, about 80 mg/L to about 150 mg/L, about 80 mg/L to about 200 mg/L, about 80 mg/L to about 500 mg/L, about 80 mg/L to about 750 mg/L, about 80 mg/L to about 1,000 mg/L, about 100 mg/L to about 150 mg/L, about 100 mg/L to about 200 mg/L, about 100 mg/L to about 500 mg/L, about 100 mg/L to about 750 mg/L, about 100 mg/L to about 1,000 mg/L, about 150 mg/L to about 200 mg/L, about 150 mg/L to about 500 mg/L, about 150 mg/L to about 750 mg/L, about 150 mg/L to about 1,000 mg/L, about 200 mg/L to about 500 mg/L, about 200 mg/L to about 750 mg/L, about 200 mg/L to about 1,000 mg/L, about 500 mg/L to about 750 mg/L, about 500 mg/L to about 1,000 mg/L, or about 750 mg/L to about 1,000 mg/L.

In some embodiments, the product outflow stream may comprise an electrical conductivity. The electrical conductivity of the product outflow stream may be at least about 0.01 mS/cm, at least about 0.05 mS/cm, at least about 0.1 mS/cm, at least about 0.5 mS/cm, at least about 1.0 mS/cm, at least about 1.5 mS/cm, at least about 2.0 mS/cm, at least about 2.5 mS/cm, at least about 3.0 mS/cm, at least about 4.0 mS/cm, at least about 5.0 mS/cm, at least about 10.0 mS/cm, or greater than about 10.0 mS/cm. The electrical conductivity of the product outflow stream may be at most about 10.0 mS/cm, at most about 5.0 mS/cm, at most about 4.0 mS/cm, at most about 3.0 mS/cm, at most about 2.5 mS/cm, at most about 2.0 mS/cm, at most about 1.5 mS/cm, at most about 1.0 mS/cm, at most about 0.5 mS/cm, at most about 0.1 mS/cm, at most about 0.05 mS/cm, at most about 0.01 mS/cm, or less than about 0.01 mS/cm. The electrical conductivity of the product outflow stream may be between about 0.01 mS/cm to about 10 mS/cm. The electrical conductivity of the product outflow stream may be between about 0.01 mS/cm to about 0.05 mS/cm, about 0.01 mS/cm to about 0.1 mS/cm, about 0.01 mS/cm to about 0.5 mS/cm, about 0.01 mS/cm to about 1 mS/cm, about 0.01 mS/cm to about 1.5 mS/cm, about 0.01 mS/cm to about 2 mS/cm, about 0.01 mS/cm to about 2.5 mS/cm, about 0.01 mS/cm to about 3 mS/cm, about 0.01 mS/cm to about 4 mS/cm, about 0.01 mS/cm to about 5 mS/cm, about 0.01 mS/cm to about 10 mS/cm, about 0.05 mS/cm to about 0.1 mS/cm, about 0.05 mS/cm to about 0.5 mS/cm, about 0.05 mS/cm to about 1 mS/cm, about 0.05 mS/cm to about 1.5 mS/cm, about 0.05 mS/cm to about 2 mS/cm, about 0.05 mS/cm to about 2.5 mS/cm, about 0.05 mS/cm to about 3 mS/cm, about 0.05 mS/cm to about 4 mS/cm, about 0.05 mS/cm to about 5 mS/cm, about 0.05 mS/cm to about 10 mS/cm, about 0.1 mS/cm to about 0.5 mS/cm, about 0.1 mS/cm to about 1 mS/cm, about 0.1 mS/cm to about 1.5 mS/cm, about 0.1 mS/cm to about 2 mS/cm, about 0.1 mS/cm to about 2.5 mS/cm, about 0.1 mS/cm to about 3 mS/cm, about 0.1 mS/cm to about 4 mS/cm, about 0.1 mS/cm to about 5 mS/cm, about 0.1 mS/cm to about 10 mS/cm, about 0.5 mS/cm to about 1 mS/cm, about 0.5 mS/cm to about 1.5 mS/cm, about 0.5 mS/cm to about 2 mS/cm, about 0.5 mS/cm to about 2.5 mS/cm, about 0.5 mS/cm to about 3 mS/cm, about 0.5 mS/cm to about 4 mS/cm, about 0.5 mS/cm to about 5 mS/cm, about 0.5 mS/cm to about 10 mS/cm, about 1 mS/cm to about 1.5 mS/cm, about 1 mS/cm to about 2 mS/cm, about 1 mS/cm to about 2.5 mS/cm, about 1 mS/cm to about 3 mS/cm, about 1 mS/cm to about 4 mS/cm, about 1 mS/cm to about 5 mS/cm, about 1 mS/cm to about 10 mS/cm, about 1.5 mS/cm to about 2 mS/cm, about 1.5 mS/cm to about 2.5 mS/cm, about 1.5 mS/cm to about 3 mS/cm, about 1.5 mS/cm to about 4 mS/cm, about 1.5 mS/cm to about 5 mS/cm, about 1.5 mS/cm to about 10 mS/cm, about 2 mS/cm to about 2.5 mS/cm, about 2 mS/cm to about 3 mS/cm, about 2 mS/cm to about 4 mS/cm, about 2 mS/cm to about 5 mS/cm, about 2 mS/cm to about 10 mS/cm, about 2.5 mS/cm to about 3 mS/cm, about 2.5 mS/cm to about 4 mS/cm, about 2.5 mS/cm to about 5 mS/cm, about 2.5 mS/cm to about 10 mS/cm, about 3 mS/cm to about 4 mS/cm, about 3 mS/cm to about 5 mS/cm, about 3 mS/cm to about 10 mS/cm, about 4 mS/cm to about 5 mS/cm, about 4 mS/cm to about 10 mS/cm, or about 5 mS/cm to about 10 mS/cm.

In some embodiments, reactors may be arranged so that fluid can flow from an outflow port (e.g., a product outflow port) of a reactor into an adjacent reactor or tank. Fluid from near the top of the working fluid in a reactor may flow into the next reactor continuously. Fluid from the middle of a reactor may flow into the next reactor continuously. Fluid from the bottom of a reactor may flow into the next reactor continuously. Fluid may also be reintroduced from any outflow source into the same reactor. In some embodiments, an outflow port (e.g., a product outflow port) is between 0.1 and 35 inches below the top of the working fluid within a reactor. In some embodiments, an outflow port (e.g., a product outflow port) is at least about 1 inch, at least about 2 inches, at least about 5 inches, at least about 10 inches, at least about 20 inches, at least about 50 inches, at least about 100 inches, at least about 250 inches, at least about 500 inches, at least about 750 inches, at least about 1,000 inches, at least about 2,500 inches, at least about 5,000 inches, at least about 7,500 inches, or at least about 10,000 inches below the top of the working fluid within a reactor. In some embodiments, an outflow port (e.g., a product outflow port) is at most about 10,000 inches, at most about 7,500 inches, at most about 5,000 inches, at most about 2,500 inches, at most about 1,000 inches, at most about 750 inches, at most about 500 inches, at most about 250 inches, at most about 100 inches, at most about 50 inches, at most about 20 inches, at most about 10 inches, at most about 5 inches, at most about 2 inches, or at most about 1 inch below the top of the working fluid within a reactor. In some embodiments, the rate of outflowing product from a bioreactor system may match the rate of inflowing feedstock, providing for a hydraulically balanced flow throughout the system. A reactor within the system may have a unique, stable microbial consortium with distinct physiological characteristics and digestion capabilities as compared to consortia in other tanks in the system. A reactor within the system may have the same microbial consortium with similar physiological characteristics and digestion capabilities as another reactor within the system. Each reactor within the system may have the same volume capacity. Each reactor within the system may have a different volume capacity. The bioreactor system may comprise at least two reactors. The bioreactor system may comprise 2 reactors, 3 reactors, 4 reactors, 5 reactors, 6 reactors, 7 reactors, 8 reactors, 9 reactors, 10 reactors, or more reactors. Reactors of a bioreactor system may be arranged as a serialized assembly of reactors. A serialized assembly of reactors may have conduits (e.g., ports or outputs) connecting each reactor to an adjacent reactor and/or container. A serialized assembly of reactors may have a continuous flow of working fluid through each reactor to the adjacent reactor.

In some embodiments, a reactor may have a single in-flow port and a single outflow port. In some embodiments, a reactor may have two or more in-flow ports and outflow ports. In some embodiments, a reactor may have a single in-flow port and two or more outflow ports. In some embodiments, a reactor may have two or more in-flow ports and a single outflow port. A reactor may have another in-flow port to provide a carbon source and/or consortium inoculum. An in-flow port may be present at any location of a reactor of the bioreactor system. An in-flow port may be present at the top of the reactor or at the bottom of the reactor. An outflow port may be present at the top of the reactor or at the bottom of the reactor. In some embodiments, the outflow ports or in-flow ports described herein comprise one or more pipes, one or more pumps, one or more ventilations, or other systems for transferring fluid from one vessel to another.

A reactor may have a single fluid connection. A reactor may have two or more fluid connections. The fluid connections may be present at the top of the working fluid in each reactor or near the top of the working fluid in each reactor. In some embodiments, the reactor may have flow from the bottom of the container back to the top to prevent build-up of sludge in the bottom of the reactor. In some embodiments, a reactor may have one or more stirrers in the bottom of the container. In some embodiments, a reactor may have one or more wipers in the bottom of the container. The wipers may stir the feedstock and prevent clogging within the reactor. The wipers may fold floc and ease floc return. The wipers (e.g., folding wipers) can fold the floc portion of the clarifier working fluid in a bottom portion of the clarifier container.

In some embodiments, the reactors may comprise one or more packed bed reactors. In some embodiments, each of the packed bed reactors has an open cell design to allow free movement of working fluid. A fixed media (e.g., scaffolding) may be secured to the inside of each packed bed reactor. The fixed media comprises materials that increase the contact surface area for the communities of microbes with working fluid. The fixed media also provides a stable platform for anchoring biofilm. The packed bed reactors may be packed with scaffolding to increase surface area within the reactor. Without wishing to be bound by theory, the increased surface area provided by the scaffolding can provide increased contact for biofilm (e.g., microbes of a microbial consortium, an established microbial strain, or any combination thereof) to grow. The scaffolding within the reactor may increase biofilm. The packed bed reactor may improve contact between the biofilm and substrates within the reactor. The fixed media can be of several types, including durable plastic, polyvinyl chloride (PVC), metal, metal alloy, glass, glass compounds, fiberglass, any suitably robust inert material, or any combination thereof. The design and configuration of the fixed media can assume various geometric patterns that allow working fluid to freely move through each packed bed reactor and prevents fouling. In some embodiments, free flow supports can control hydraulic shearing which in time promotes even distribution of working fluid. In some embodiments, the fixed media is dispersed throughout a cross sectional area of each packed bed reactor.

The scaffolding may comprise one or more tubes, one or more rings, or other packing materials. In some embodiments, the packed bed reactors provided herein may comprise a bundle of tubes or columns. In some embodiments, the scaffolding may comprise hexagonal, grid-like, perforated tubing, or any combination thereof. Without wishing to be bound by theory, hexagonal, grid-like, and/or perforated scaffolding can increase the surface area and the flow through the columns within the container. In some embodiments, the tubes or columns of the scaffolding can comprise a diameter between 0.25 and 50 inches. In some embodiments, the scaffolding can comprise a diameter of at least about 0.20 inches, at least about 0.25 inches, at least about 0.3 inches, at least about 0.4 inches, at least about 0.5 inches, at least about 0.6 inches, at least about 0.7 inches, at least about 0.8 inches, at least about 0.9 inches, at least about 1 inch, at least about 2 inches, at least about 3 inches, at least about 4 inches, at least about 5 inches, at least about 10 inches, at least about 15 inches, at least about 20 inches, at least about 25 inches, at least about 30 inches, at least about 35 inches, at least about 40 inches, at least about 45 inches, at least about 50 inches, at least about 55 inches, at least about 60 inches, at least about 65 inches, at least about 70 inches, or at least about 75 inches. In some embodiments, the scaffolding can comprise a diameter of at most about 75 inches, at most about 70 inches, at most about 65 inches, at most about 60 inches, at most about 55 inches, at most about 50 inches, at most about 45 inches, at most about 40 inches, at most about 35 inches, at most about 30 inches, at most about 25 inches, at most about 20 inches, at most about 15 inches, at most about 10 inches, at most about 5 inches, at most about 4 inches, at most about 3 inches, at most about 2 inches, at most about 1 inch, at most about 0.9 inches, at most about 0.8 inches, at most about 0.7 inches, at most about 0.6 inches, at most about 0.5 inches, at most about 0.4 inches, at most about 0.3 inches, at most about 0.25 inches, or at most about 0.2 inches. Without wishing to be bound by theory, a system with packed bed reactors may improve production of bacterial isolates or other microbes. In some embodiments, reactors without scaffolding (i.e., reactors that are not packed bed reactors) improve production of bacterial isolates or other microbes or improve digestion of digestible substrates.

In some embodiments, the reactors may comprise one or more fluidized bed reactors. In fluidized bed reactors, solid particles may be circulated within working fluid of the reactors, which may provide a surface for microbial colonization. Such particles may comprise, for example, particles of an inorganic substrate such as rock phosphate particles. In some embodiments, the fluidized bed reactors may be the same volume. In some embodiments, the fluidized bed reactors may be different volumes. In some embodiments, the fluidized bed reactors increase uniformity of particle mixing within the bioreactor system. The solid material of the fluidized bed reactor can have intrinsic fluid-like properties and allow for a more complete mixing. Reduction or elimination of radial and axial concentration gradients can provide for better fluid-solid contact and can achieve better uniformity of particle mixing. In some embodiments, the fluidized bed reactors increase the uniformity of temperature gradients within the bioreactor system. Without wishing to be bound by theory, the open container of the fluidized bed reactor can provide for a reduction in isolated hot or cold spots that may develop during mixing of a working fluid in the container, allowing for a uniform temperature distribution of the fluid. Hot spots can be areas of localized temperature changes during heating of a working fluid. Cold spots can be areas of localized temperature changes during cooling of a working fluid. The reduction in isolated temperature changes from the configuration of the fluidized bed reactor can provide for a more uniform temperature distribution of the fluid.

The flow rate of the bioreactor system may be chosen to allow for sufficient dwell time of a working fluid within each of the reactors for a stable and unique microbial consortium to form within each of the reactors. In some embodiments, working fluid in each reactor is continuously recycled at a rate ratio in a range of approximately 25:1 to 35:1, 25 to 35 gallons per minute of the recycle rate to one gallon per minute of the hydraulic feed rate. In some embodiments, working fluid in each reactor is continuously recycled at a rate ratio of at least about 10:1, at least about 12:1, at least about 14:1, at least about 16:1, at least about 18:1, at least about 20:1, at least about 22:1, at least about 24:1, at least about 26:1, at least about 28:1, at least about 30:1, at least about 32:1, at least about 34:1, at least about 36:1, at least about 38:1, at least about 40:1, at least about 45:1, or at least about 50:1. In some embodiments, working fluid in each reactor is continuously recycled at a rate ratio of at most about 50:1 at most about 45:1, at most about 40:1, at most about 38:1, at most about 36:1, at most about 34:1, at most about 32:1, at most about 30:1, at most about 28:1, at most about 26:1, at most about 24:1, at most about 22:1, at most about 20:1, at most about 18:1, at most about 16:1, at most about 14:1, at most about 12:1, or at most about 10:1. Working fluid may be recycled by one or more pumps to prevent solids settling and to provide sufficient velocity and hydraulic shear to prevent excessive buildup and sloughing of biofilm. A digestion system provided herein may comprise a first flow rate, second flow rate, third flow rate, fourth flow rate, fifth flow rate, sixth flow rate, or seventh flow rate.

Reactors can be maintained at specific temperatures which may aid in digestion and growth of microbial consortia within the system. In some embodiments, a temperature of a reactor is at least about 15° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 35° C., at least about 40° C., at least about 45° C., or at least about 50° C. In some embodiments, a temperature of a reactor is at most about 50° C., at most about 45° C., at most about 40° C., at most about 35° C., at most about 30° C., at most about 29° C., at most about 28° C., at most about 27° C., at most about 26° C., at most about 25° C., at most about 24° C., at most about 23° C., at most about 22° C., at most about 21° C., at most about 20° C., or at most about 15° C.

In some embodiments, a temperature of a reactor is about 15° C. to about 45° C. In some embodiments, a temperature of a reactor is about 15° C. to about 20° C., about 15° C. to about 22° C., about 15° C. to about 24° C., about 15° C. to about 26° C., about 15° C. to about 28° C., about 15° C. to about 30° C., about 15° C. to about 32° C., about 15° C. to about 34° C., about 15° C. to about 36° C., about 15° C. to about 40° C., about 15° C. to about 45° C., about 20° C. to about 22° C., about 20° C. to about 24° C., about 20° C. to about 26° C., about 20° C. to about 28° C., about 20° C. to about 30° C., about 20° C. to about 32° C., about 20° C. to about 34° C., about 20° C. to about 36° C., about 20° C. to about 40° C., about 20° C. to about 45° C., about 22° C. to about 24° C., about 22° C. to about 26° C., about 22° C. to about 28° C., about 22° C. to about 30° C., about 22° C. to about 32° C., about 22° C. to about 34° C., about 22° C. to about 36° C., about 22° C. to about 40° C., about 22° C. to about 45° C., about 24° C. to about 26° C., about 24° C. to about 28° C., about 24° C. to about 30° C., about 24° C. to about 32° C., about 24° C. to about 34° C., about 24° C. to about 36° C., about 24° C. to about 40° C., about 24° C. to about 45° C., about 26° C. to about 28° C., about 26° C. to about 30° C., about 26° C. to about 32° C., about 26° C. to about 34° C., about 26° C. to about 36° C., about 26° C. to about 40° C., about 26° C. to about 45° C., about 28° C. to about 30° C., about 28° C. to about 32° C., about 28° C. to about 34° C., about 28° C. to about 36° C., about 28° C. to about 40° C., about 28° C. to about 45° C., about 30° C. to about 32° C., about 30° C. to about 34° C., about 30° C. to about 36° C., about 30° C. to about 40° C., about 30° C. to about 45° C., about 32° C. to about 34° C., about 32° C. to about 36° C., about 32° C. to about 40° C., about 32° C. to about 45° C., about 34° C. to about 36° C., about 34° C. to about 40° C., about 34° C. to about 45° C., about 36° C. to about 40° C., about 36° C. to about 45° C., or about 40° C. to about 45° C.

In some embodiments, the high aeration of the working fluid of a bioreactor system described herein can help enrich microbes with a targeted functionality (e.g., phosphate solubilizers, zinc solubilizers, nitrogen use efficiency). Reactors may be maintained under aerobic, microaerobic, or anaerobic conditions. The series of reactors in a bioreactor system may have different aerobic conditions. The series of reactors in a bioreactor system may have the same aerobic conditions. In some embodiments, a reactor may have the same aerobic condition as an adjacent reactor. In some embodiments, a reactor may have a different aerobic condition than an adjacent reactor. In some embodiments, a bioreactor system may have aerobic, microaerobic, anaerobic conditions, or any combination thereof.

In some embodiments, aerobic conditions comprise conditions with a dissolved oxygen measurement of greater than 2 mg/L. In some embodiments, aerobic conditions comprise conditions with a dissolved oxygen measurement of at least about 2 mg/L mg/L, at least about 3 mg/L mg/L, at least about 4 mg/L, at least about 5 mg/L, at least about 6 mg/L, at least about 7 mg/L, at least about 8 mg/L, at least about 9 mg/L, at least about 10 mg/L, at least about 12 mg/L, at least about 14 mg/L, at least about 15 mg/L, or greater than about 15 mg/L. In some embodiments, aerobic conditions comprise conditions with a dissolved oxygen measurement from about 2 mg/L to about 15 mg/L. In some embodiments, aerobic conditions comprise conditions with a dissolved oxygen measurement from about 2 mg/L to about 3 mg/L, about 2 mg/L to about 4 mg/L, about 2 mg/L to about 5 mg/L, about 2 mg/L to about 6 mg/L, about 2 mg/L to about 7 mg/L, about 2 mg/L to about 8 mg/L, about 2 mg/L to about 9 mg/L, about 2 mg/L to about 10 mg/L, about 2 mg/L to about 12 mg/L, about 2 mg/L to about 14 mg/L, about 2 mg/L to about 15 mg/L, about 3 mg/L to about 4 mg/L, about 3 mg/L to about 5 mg/L, about 3 mg/L to about 6 mg/L, about 3 mg/L to about 7 mg/L, about 3 mg/L to about 8 mg/L, about 3 mg/L to about 9 mg/L, about 3 mg/L to about 10 mg/L, about 3 mg/L to about 12 mg/L, about 3 mg/L to about 14 mg/L, about 3 mg/L to about 15 mg/L, about 4 mg/L to about 5 mg/L, about 4 mg/L to about 6 mg/L, about 4 mg/L to about 7 mg/L, about 4 mg/L to about 8 mg/L, about 4 mg/L to about 9 mg/L, about 4 mg/L to about 10 mg/L, about 4 mg/L to about 12 mg/L, about 4 mg/L to about 14 mg/L, about 4 mg/L to about 15 mg/L, about 5 mg/L to about 6 mg/L, about 5 mg/L to about 7 mg/L, about 5 mg/L to about 8 mg/L, about 5 mg/L to about 9 mg/L, about 5 mg/L to about 10 mg/L, about 5 mg/L to about 12 mg/L, about 5 mg/L to about 14 mg/L, about 5 mg/L to about 15 mg/L, about 6 mg/L to about 7 mg/L, about 6 mg/L to about 8 mg/L, about 6 mg/L to about 9 mg/L, about 6 mg/L to about 10 mg/L, about 6 mg/L to about 12 mg/L, about 6 mg/L to about 14 mg/L, about 6 mg/L to about 15 mg/L, about 7 mg/L to about 8 mg/L, about 7 mg/L to about 9 mg/L, about 7 mg/L to about 10 mg/L, about 7 mg/L to about 12 mg/L, about 7 mg/L to about 14 mg/L, about 7 mg/L to about 15 mg/L, about 8 mg/L to about 9 mg/L, about 8 mg/L to about 10 mg/L, about 8 mg/L to about 12 mg/L, about 8 mg/L to about 14 mg/L, about 8 mg/L to about 15 mg/L, about 9 mg/L to about 10 mg/L, about 9 mg/L to about 12 mg/L, about 9 mg/L to about 14 mg/L, about 9 mg/L to about 15 mg/L, about 10 mg/L to about 12 mg/L, about 10 mg/L to about 14 mg/L, about 10 mg/L to about 15 mg/L, about 12 mg/L to about 14 mg/L, about 12 mg/L to about 15 mg/L, or about 14 mg/L to about 15 mg/L. In some embodiments, aerobic conditions comprise conditions with a dissolved oxygen measurement of between 2 mg/L and 10 mg/L. In some embodiments, microaerobic conditions comprise conditions with a dissolved oxygen measurement of less than 2 mg/L. In some embodiments, microaerobic conditions comprise conditions with a dissolved oxygen measurement of at most about 1.99 mg/L, at most about 1.8 mg/L, at most about 1.6 mg/L, at most about 1.5 mg/L, at most about 1.4 mg/L, at most about 1.3 mg/L, at most about 1.2 mg/L, at most about 1.1 mg/L, at most about 1 mg/L, at most about 0.9 mg/L, at most about 0.8 mg/L, at most about 0.7 mg/L, at most about 0.6 mg/L, at most about 0.5 mg/L, at most about 0.4 mg/L, at most about 0.3 mg/L, at most about 0.2 mg/L, at most about 0.1 mg/L, or less than about 0.1 mg/L but not 0 mg/L. In some embodiments, microaerobic conditions comprise conditions with a dissolved oxygen measurement from about 0.1 mg/L to about 1.99 mg/L. In some embodiments, microaerobic conditions comprise conditions with a dissolved oxygen measurement from about 0.1 mg/L to about 0.2 mg/L, about 0.1 mg/L to about 0.3 mg/L, about 0.1 mg/L to about 0.4 mg/L, about 0.1 mg/L to about 0.5 mg/L, about 0.1 mg/L to about 0.8 mg/L, about 0.1 mg/L to about 1 mg/L, about 0.1 mg/L to about 1.2 mg/L, about 0.1 mg/L to about 1.4 mg/L, about 0.1 mg/L to about 1.6 mg/L, about 0.1 mg/L to about 1.8 mg/L, about 0.1 mg/L to about 1.99 mg/L, about 0.2 mg/L to about 0.3 mg/L, about 0.2 mg/L to about 0.4 mg/L, about 0.2 mg/L to about 0.5 mg/L, about 0.2 mg/L to about 0.8 mg/L, about 0.2 mg/L to about 1 mg/L, about 0.2 mg/L to about 1.2 mg/L, about 0.2 mg/L to about 1.4 mg/L, about 0.2 mg/L to about 1.6 mg/L, about 0.2 mg/L to about 1.8 mg/L, about 0.2 mg/L to about 1.99 mg/L, about 0.3 mg/L to about 0.4 mg/L, about 0.3 mg/L to about 0.5 mg/L, about 0.3 mg/L to about 0.8 mg/L, about 0.3 mg/L to about 1 mg/L, about 0.3 mg/L to about 1.2 mg/L, about 0.3 mg/L to about 1.4 mg/L, about 0.3 mg/L to about 1.6 mg/L, about 0.3 mg/L to about 1.8 mg/L, about 0.3 mg/L to about 1.99 mg/L, about 0.4 mg/L to about 0.5 mg/L, about 0.4 mg/L to about 0.8 mg/L, about 0.4 mg/L to about 1 mg/L, about 0.4 mg/L to about 1.2 mg/L, about 0.4 mg/L to about 1.4 mg/L, about 0.4 mg/L to about 1.6 mg/L, about 0.4 mg/L to about 1.8 mg/L, about 0.4 mg/L to about 1.99 mg/L, about 0.5 mg/L to about 0.8 mg/L, about 0.5 mg/L to about 1 mg/L, about 0.5 mg/L to about 1.2 mg/L, about 0.5 mg/L to about 1.4 mg/L, about 0.5 mg/L to about 1.6 mg/L, about 0.5 mg/L to about 1.8 mg/L, about 0.5 mg/L to about 1.99 mg/L, about 0.8 mg/L to about 1 mg/L, about 0.8 mg/L to about 1.2 mg/L, about 0.8 mg/L to about 1.4 mg/L, about 0.8 mg/L to about 1.6 mg/L, about 0.8 mg/L to about 1.8 mg/L, about 0.8 mg/L to about 1.99 mg/L, about 1 mg/L to about 1.2 mg/L, about 1 mg/L to about 1.4 mg/L, about 1 mg/L to about 1.6 mg/L, about 1 mg/L to about 1.8 mg/L, about 1 mg/L to about 1.99 mg/L, about 1.2 mg/L to about 1.4 mg/L, about 1.2 mg/L to about 1.6 mg/L, about 1.2 mg/L to about 1.8 mg/L, about 1.2 mg/L to about 1.99 mg/L, about 1.4 mg/L to about 1.6 mg/L, about 1.4 mg/L to about 1.8 mg/L, about 1.4 mg/L to about 1.99 mg/L, about 1.6 mg/L to about 1.8 mg/L, about 1.6 mg/L to about 1.99 mg/L, or about 1.8 mg/L to about 1.99 mg/L. In some embodiments, anaerobic conditions comprise conditions with a dissolved oxygen measurement of 0 mg/L.

In some embodiments, the reactors comprise high-speed mixers to move a working fluid and increase aeration. The mixers may facilitate an aerobic digestion and enrich microbial communities with a target functionality (e.g., phosphate solubilizing microbes). The high-speed mixers can use a speed comprising at least about 1000 RPM, 1100 RPM, 1200 RPM, 1300 RPM, 1400 RPM, 1500 RPM, 1600 RPM, 1700 RPM, 1800 RPM, 1900 RPM, 2000 RPM, or greater than about 2000 RPM.

Reactors of a bioreactor system described herein may comprise a working volume used to hold a volume of working fluid. A working volume of a reactor of a bioreactor system described herein may be at least about 5 gallons, at least about 10 gallons, at least about 20 gallons, at least about 50 gallons, at least about 75 gallons, at least about 100 gallons, at least about 250 gallons, at least about 500 gallons, at least about 750 gallons, at least about 1,000 gallons, at least about 2,000 gallons, at least about 3,000 gallons, at least about 4,000 gallons, at least about 5,000 gallons, at least about 7,500 gallons, at least about 10,000 gallons, at least about 15,000 gallons, at least about 20,000 gallons, at least about 50,000 gallons, or more than about 50,000 gallons. A working volume of a reactor of a bioreactor system described herein may be at most about 50,000 gallons, at most about 20,000 gallons, at most about 15,000 gallons, at most about 10,000 gallons, at most about 7,500 gallons, at most about 5,000 gallons, at most about 4,000 gallons, at most about 3,000 gallons, at most about 2,000 gallons, at most about 1,000 gallons, at most about 750 gallons, at most about 500 gallons, at most about 250 gallons, at most about 100 gallons, at most about 75 gallons, at most about 50 gallons, at most about 20 gallons, at most about 10 gallons, at most about 5 gallons, or less than about 5 gallons.

Reactors may be maintained at different pH levels within a bioreactor system. Reactors may be maintained at the same pH levels within a bioreactor system. The pH of a reactor in a bioreactor system may be at least about 4.0, at least about 4.5, at least about 5.0, at least about 5.5, at least about 6.0, at least about 6.5, at least about 7.0, at least about 7.5, at least about 8.0, at least about 8.5, at least about 9.0, at least about 9.5, or at least about 10.0. In some embodiments, the pH of a reactor in a bioreactor system may be at most about 10.0, at most about 9.5, at most about 9.0, at most about 8.5, at most about 8.0, at most about 7.5, at most about 7.0, at most about 6.5, at most about 6.0, at most about 5.5, at most about 5.0, at most about 4.5, or at most about 4.0.

In some embodiments, the pH of a reactor in a bioreactor system may be about 3 to about 9. In some embodiments, the pH of a reactor in a bioreactor system may be about 3 to about 3.5, about 3 to about 4, about 3 to about 4.5, about 3 to about 5, about 3 to about 5.5, about 3 to about 6, about 3 to about 6.5, about 3 to about 7, about 3 to about 7.5, about 3 to about 8, about 3 to about 9, about 3.5 to about 4, about 3.5 to about 4.5, about 3.5 to about 5, about 3.5 to about 5.5, about 3.5 to about 6, about 3.5 to about 6.5, about 3.5 to about 7, about 3.5 to about 7.5, about 3.5 to about 8, about 3.5 to about 9, about 4 to about 4.5, about 4 to about 5, about 4 to about 5.5, about 4 to about 6, about 4 to about 6.5, about 4 to about 7, about 4 to about 7.5, about 4 to about 8, about 4 to about 9, about 4.5 to about 5, about 4.5 to about 5.5, about 4.5 to about 6, about 4.5 to about 6.5, about 4.5 to about 7, about 4.5 to about 7.5, about 4.5 to about 8, about 4.5 to about 9, about 5 to about 5.5, about 5 to about 6, about 5 to about 6.5, about 5 to about 7, about 5 to about 7.5, about 5 to about 8, about 5 to about 9, about 5.5 to about 6, about 5.5 to about 6.5, about 5.5 to about 7, about 5.5 to about 7.5, about 5.5 to about 8, about 5.5 to about 9, about 6 to about 6.5, about 6 to about 7, about 6 to about 7.5, about 6 to about 8, about 6 to about 9, about 6.5 to about 7, about 6.5 to about 7.5, about 6.5 to about 8, about 6.5 to about 9, about 7 to about 7.5, about 7 to about 8, about 7 to about 9, about 7.5 to about 8, about 7.5 to about 9, or about 8 to about 9.

In some embodiments, the reactors may comprise distribution components (e.g., a distribution ring) that is subsurface of the discharge volume. The distribution component can reduce the amount of surface disruption, and/or keep the environment in the reactor anaerobic. The environment of the reactor may be anaerobic, microaerobic, or aerobic. Without wishing to be bound by theory, the microaerobic conditions of the reactors of the bioreactor system may enrich certain microbes (e.g., zinc-solubilization microbes, phosphate-solubilization microbes, and/or microbes with nitrogen use efficiency capability) in the microbial consortium of the system.

A working fluid may comprise a fluidic substance that moves through a bioreactor system as described herein. A working fluid can comprise solid components, liquid components, gaseous components, or any combination thereof. A working fluid can comprise microbial consortia, isolated microbes or inoculum of a microbial strain (e.g., target isolates), additional organic and/or materials, or any combination thereof. The mixture of microbial consortia, isolated microbes (e.g., target isolates), additional organic and/or materials within a working fluid may allow for the expansion of microbes or act as a culture for an inoculum of a microbe to grow. A working fluid may comprise a pH, viscosity, temperature, surface tension, adhesion, and/or volume that enhances the growth and/or functioning of microbes or microorganisms. In some embodiments, a volume of working fluid within each reactor may be continuously replenished and drawn from. In some embodiments, a volume of working fluid within each reactor is replenished and drawn from in batches (e.g., discontinuously). In some embodiments, a working fluid in a first reactor may comprise a first working fluid. In some embodiments, a working fluid in a second reactor may comprise a second working fluid. In some embodiments, a working fluid in a third reactor may comprise a third working fluid. In some embodiments, a working fluid in a fourth reactor may comprise a fourth working fluid. In some embodiments, a working fluid in a fifth reactor may comprise a fifth working fluid. In some embodiments, at least one of the first working fluid, the second working fluid, or the third working fluid comprises a pH buffering system. A method described herein can further comprise maintaining the pH of at least one of the first working fluid, the second working fluid, the third working fluid, the fourth working fluid, or the fifth working fluid at at least about 4.0, at least about 4.5, at least about 5.0, at least about 5.5, at least about 6.0, at least about 6.5, at least about 7.0, at least about 7.5, at least about 8.0, at least about 8.5, at least about 9.0, at least about 9.5, or at least about 10.0. A method described herein can further comprise maintaining the pH of at least one of the first working fluid, the second working fluid, the third working fluid, the fourth working fluid, or the fifth working fluid at at most about 10.0, at most about 9.5, at most about 9.0, at most about 8.5, at most about 8.0, at most about 7.5, at most about 7.0, at most about 6.5, at most about 6.0, at most about 5.5, at most about 5.0, at most about 4.5, or at most about 4.0. In some embodiments, the methods described herein can comprise maintaining the pH of at least one of the first working fluid, the second working fluid, the third working fluid, the fourth working fluid, or the fifth working fluid between about 3 to about 10. In some embodiments, the methods described herein can comprise maintaining the pH of at least one of the first working fluid, the second working fluid, the third working fluid, the fourth working fluid, or the fifth working fluid between about 3 to about 4, about 3 to about 4.5, about 3 to about 5, about 3 to about 6, about 3 to about 6.5, about 3 to about 7, about 3 to about 7.5, about 3 to about 8, about 3 to about 8.5, about 3 to about 9, about 3 to about 10, about 4 to about 4.5, about 4 to about 5, about 4 to about 6, about 4 to about 6.5, about 4 to about 7, about 4 to about 7.5, about 4 to about 8, about 4 to about 8.5, about 4 to about 9, about 4 to about 10, about 4.5 to about 5, about 4.5 to about 6, about 4.5 to about 6.5, about 4.5 to about 7, about 4.5 to about 7.5, about 4.5 to about 8, about 4.5 to about 8.5, about 4.5 to about 9, about 4.5 to about 10, about 5 to about 6, about 5 to about 6.5, about 5 to about 7, about 5 to about 7.5, about 5 to about 8, about 5 to about 8.5, about 5 to about 9, about 5 to about 10, about 6 to about 6.5, about 6 to about 7, about 6 to about 7.5, about 6 to about 8, about 6 to about 8.5, about 6 to about 9, about 6 to about 10, about 6.5 to about 7, about 6.5 to about 7.5, about 6.5 to about 8, about 6.5 to about 8.5, about 6.5 to about 9, about 6.5 to about 10, about 7 to about 7.5, about 7 to about 8, about 7 to about 8.5, about 7 to about 9, about 7 to about 10, about 7.5 to about 8, about 7.5 to about 8.5, about 7.5 to about 9, about 7.5 to about 10, about 8 to about 8.5, about 8 to about 9, about 8 to about 10, about 8.5 to about 9, about 8.5 to about 10, or about 9 to about 10.

In some embodiments, a reactor (e.g., container) can comprise a temperature. The temperature of the reactor can provide conditions suitable for growth and/or survival of a microbial strain. In some embodiments, one or more microbes described herein can be psychrophiles, mesophiles, thermophiles, or any combination thereof. In some embodiments, one or more reactors can comprise mesophilic temperature conditions. In some embodiments, one or more reactors (e.g., containers) of a bioreactor system described herein may comprise a temperature of at least about 20° C., at least about 25° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 45° C., at least about 50° C., or greater than about 50° C. In some embodiments, the temperature can be at most about 45° C. In some embodiments, the temperature can be maintained in a mesophilic range (e.g., less than 45° C.). In some embodiments, the temperature may not be thermophilic (e.g., greater than 45° C.). In some embodiments, thermophilic conditions are avoided in one or more containers of a bioreactor system described herein. In some embodiments, one or more reactors (e.g., containers) of a bioreactor system described herein may comprise a temperature of at most about 50° C., at most about 45° C., at most about 40° C., at most about 39° C., at most about 38° C., at most about 37° C., at most about 36° C., at most about 35° C., at most about 34° C., at most about 33° C., at most about 32° C., at most about 31° C., at most about 30° C., at most about 25° C., at most about 20° C., or less than about 20° C. In some embodiments, one or more reactors (e.g., containers) of a bioreactor system described herein may comprise a temperature between about 25° C. to about 40° C. In some embodiments, one or more reactors (e.g., containers) of a bioreactor system described herein may comprise a temperature between about 25° C. to about 30° C., about 25° C. to about 31° C., about 25° C. to about 32° C., about 25° C. to about 32° C., about 25° C. to about 34° C., about 25° C. to about 35° C., about 25° C. to about 36° C., about 25° C. to about 37° C., about 25° C. to about 38° C., about 25° C. to about 39° C., about 25° C. to about 40° C., about 30° C. to about 31° C., about 30° C. to about 32° C., about 30° C. to about 32° C., about 30° C. to about 34° C., about 30° C. to about 35° C., about 30° C. to about 36° C., about 30° C. to about 37° C., about 30° C. to about 38° C., about 30° C. to about 39° C., about 30° C. to about 40° C., about 31° C. to about 32° C., about 31° C. to about 32° C., about 31° C. to about 34° C., about 31° C. to about 35° C., about 31° C. to about 36° C., about 31° C. to about 37° C., about 31° C. to about 38° C., about 31° C. to about 39° C., about 31° C. to about 40° C., about 32° C. to about 32° C., about 32° C. to about 34° C., about 32° C. to about 35° C., about 32° C. to about 36° C., about 32° C. to about 37° C., about 32° C. to about 38° C., about 32° C. to about 39° C., about 32° C. to about 40° C., about 32° C. to about 34° C., about 32° C. to about 35° C., about 32° C. to about 36° C., about 32° C. to about 37° C., about 32° C. to about 38° C., about 32° C. to about 39° C., about 32° C. to about 40° C., about 34° C. to about 35° C., about 34° C. to about 36° C., about 34° C. to about 37° C., about 34° C. to about 38° C., about 34° C. to about 39° C., about 34° C. to about 40° C., about 35° C. to about 36° C., about 35° C. to about 37° C., about 35° C. to about 38° C., about 35° C. to about 39° C., about 35° C. to about 40° C., about 36° C. to about 37° C., about 36° C. to about 38° C., about 36° C. to about 39° C., about 36° C. to about 40° C., about 37° C. to about 38° C., about 37° C. to about 39° C., about 37° C. to about 40° C., about 38° C. to about 39° C., about 38° C. to about 40° C., or about 39° C. to about 40° C. In some embodiments, no reactor of the bioreactor system comprises thermophilic conditions (e.g., a temperature above about 50° C. or between 55° C. and 60° C.).

In continuous operation, each reactor may have a constant volume of working fluid therein, with the rate of fluid flowing into each reactor matching the rate of fluid flowing out of each reactor. As each reactor may comprise a different microbial consortium and have different conditions from other reactors, the working fluid within each reactor may be considered to be distinct from working fluids within the other reactors. The total volume of working fluid within a bioreactor system may be referred to herein as the “total working volume” of the bioreactor system.

A working fluid may be maintained within a reactor 110. A maintained working fluid may be a working fluid as it rests in a reactor 110 of a bioreactor system. Without wishing to be bound by theory, maintenance of a working fluid may support enrichment and/or growth of a population of the microbial strain or another component of a working fluid as described herein. In some embodiments, the volume of working fluid within the reactor 110 may be maintained at 5 gallons. In some embodiments, the volume of working fluid within the reactor may be maintained at least about 1 gallon, at least about 2 gallons, at least about 3 gallons, at least about 4 gallons, at least about 5 gallons, at least about 6 gallons, at least about 7 gallons, at least about 10 gallons, at least about 15 gallons, at least about 20 gallons, at least about 25 gallons, at least about 30 gallons, at least about 50 gallons, at least about 75 gallons, at least about 100 gallons, at least about 250 gallons, at least about 500 gallons, at least about 1,000 gallons, at least about 2,500 gallons, at least about 5,000 gallons, at least about 10,000 gallons, at least about 12,000 gallons, at least about 15,000 gallons, or more than about 15,000 gallons. In some embodiments, the volume of working fluid within the reactor may be maintained at most about 15,000 gallons, at most about 12,000 gallons, at most about 10,000 gallons, at most about 5,000 gallons, at most about 2,500 gallons, at most about 1,000 gallons, at most about 500 gallons, at most about 250 gallons, at most about 100 gallons, at most about 75 gallons, at most about 50 gallons, at most about 30 gallons, at most about 25 gallons, at most about 20 gallons, at most about 15 gallons, at most about 10 gallons, at most about 7 gallons, at most about 6 gallons, at most about 5 gallons, at most about 4 gallons, at most about 3 gallons, at most about 2 gallons, at most about 1 gallon, or less than about 1 gallon.

In some embodiments, the volume of working fluid within the reactor may be maintained from about 5 gallons to about 10,000 gallons. In some embodiments, the volume of working fluid within the reactor may be maintained from about 5 gallons to about 10 gallons, about 5 gallons to about 25 gallons, about 5 gallons to about 50 gallons, about 5 gallons to about 75 gallons, about 5 gallons to about 100 gallons, about 5 gallons to about 150 gallons, about 5 gallons to about 500 gallons, about 5 gallons to about 1,000 gallons, about 5 gallons to about 2,500 gallons, about 5 gallons to about 5,000 gallons, about 5 gallons to about 10,000 gallons, about 10 gallons to about 25 gallons, about 10 gallons to about 50 gallons, about 10 gallons to about 75 gallons, about 10 gallons to about 100 gallons, about 10 gallons to about 150 gallons, about 10 gallons to about 500 gallons, about 10 gallons to about 1,000 gallons, about 10 gallons to about 2,500 gallons, about 10 gallons to about 5,000 gallons, about 10 gallons to about 10,000 gallons, about 25 gallons to about 50 gallons, about 25 gallons to about 75 gallons, about 25 gallons to about 100 gallons, about 25 gallons to about 150 gallons, about 25 gallons to about 500 gallons, about 25 gallons to about 1,000 gallons, about 25 gallons to about 2,500 gallons, about 25 gallons to about 5,000 gallons, about 25 gallons to about 10,000 gallons, about 50 gallons to about 75 gallons, about 50 gallons to about 100 gallons, about 50 gallons to about 150 gallons, about 50 gallons to about 500 gallons, about 50 gallons to about 1,000 gallons, about 50 gallons to about 2,500 gallons, about 50 gallons to about 5,000 gallons, about 50 gallons to about 10,000 gallons, about 75 gallons to about 100 gallons, about 75 gallons to about 150 gallons, about 75 gallons to about 500 gallons, about 75 gallons to about 1,000 gallons, about 75 gallons to about 2,500 gallons, about 75 gallons to about 5,000 gallons, about 75 gallons to about 10,000 gallons, about 100 gallons to about 150 gallons, about 100 gallons to about 500 gallons, about 100 gallons to about 1,000 gallons, about 100 gallons to about 2,500 gallons, about 100 gallons to about 5,000 gallons, about 100 gallons to about 10,000 gallons, about 150 gallons to about 500 gallons, about 150 gallons to about 1,000 gallons, about 150 gallons to about 2,500 gallons, about 150 gallons to about 5,000 gallons, about 150 gallons to about 10,000 gallons, about 500 gallons to about 1,000 gallons, about 500 gallons to about 2,500 gallons, about 500 gallons to about 5,000 gallons, about 500 gallons to about 10,000 gallons, about 1,000 gallons to about 2,500 gallons, about 1,000 gallons to about 5,000 gallons, about 1,000 gallons to about 10,000 gallons, about 2,500 gallons to about 5,000 gallons, about 2,500 gallons to about 10,000 gallons, or about 5,000 gallons to about 10,000 gallons.

A first container may comprise an outlet port. The outlet port can be fluidly connected to an inlet port of another container of the bioreactor system (e.g., a second container). The second container may comprise an outlet port. The outlet port of the second container can be fluidly connected to an inlet port of another container of the bioreactor system (e.g., a third container). In some embodiments, an inlet port of a container (e.g., a first container) may be at a higher level of the container than an outlet port of the container. A container (e.g., a third container) can comprise an outlet port fluidly connected to a clarifier container. The outlet port of a container may be fluidly connected to an input port of the clarifier container. A method described herein may further comprise maintaining the volume of one or more working fluids for the duration of time (e.g., for the duration of time of operating the bioreactor system. For example, the method may further comprise maintaining the volume of a first working fluid, a second working fluid, a third working fluid, a fourth working fluid, a fifth working fluid, or any combination thereof throughout the duration of time.

In some embodiments, the working fluid in the reactor 110 may be recirculated (e.g., recycled) within the reactor of the bioreactor system 100. Working fluid may be recirculated via a conduit or output of a reactor and reintroduced into the same reactor. Without wishing to be bound by theory, recirculation of a working fluid within a reactor may support enrichment and/or growth of a population of the microbial strain or another component of a working fluid as described herein. In some embodiments, the rate of recirculation within the reactor may be between 4-9 gallons/min. In some embodiments, the rate of recirculation within the reactor 110 may be at least about 0.5 gallon/min, at least about 1 gallon/min, at least about 2 gallons/min, at least about 3 gallons/min, at least about 3.5 gallons/min, at least about 4 gallons/min, at least about 4.5 gallons/min, at least about 5 gallons/min, at least about 5.5 gallons/min, at least about 6 gallons/min, at least about 6.5 gallons/min, at least about 7 gallons/min, at least about 7.5 gallons/min, at least about 8 gallons/min, at least about 8.5 gallons/min, at least about 9 gallons/min, at least about 10 gallons/min, at least about 11 gallons/min, at least about 13 gallons/min, at least about 15 gallons/min, at least about 20 gallons/min, at least about 25 gallons/min, at least about 30 gallons/min, at least about 40 gallons/min, or at least about 50 gallons/min.

In some embodiments, at least a portion of the working fluid from one reactor may be transferred to another reactor (e.g., the subsequent reactor). In some embodiments, at least a portion of the first working fluid may be transferred to the second reactor. In some embodiments, at least a portion of the second working fluid may be transferred to the third reactor. In some embodiments, at least a portion of the third working fluid may be transferred to the fourth reactor. In some embodiments, at least a portion of the fourth working fluid may be transferred to the fifth reactor. In some embodiments, a working fluid may be mixed in a reactor (e.g., chamber or container) prior to a first reactor. In some embodiments, the working fluid in each reactor may be distinct from the working fluid in other reactors in the bioreactor system. Distinct working fluids may comprise different microbial populations. The different microbial populations may comprise different microbes, (e.g., bacteria, fungi, algae, or any combination thereof). Distinct working fluids may comprise different concentrations of a target isolate. Distinct working fluids may comprise different concentrations of a carbon source and/or a nitrogen source. Distinct working fluids may comprise different microbial populations, different combinations and/or concentrations of a target isolate, different combinations and/or concentrations of a carbon source, different combinations and/or concentrations of a nitrogen source, or any combination thereof. The working fluid in a reactor of a bioreactor system may be similar to a working fluid of a different reactor of the bioreactor system.

The working fluid within each reactor may comprise different enzymes, which may be produced by microbes within the working fluid. An enzyme within a working fluid may comprise a dehydrogenase, a hydrogenase, an oxidase, a catalase, a peroxidase, a phenol o-hydroxylase, a dextransucrase, an aminotransferase, a rhodanese, a carboxylesterase, a lipase, a phosphatase, a nuclease, a phytase, an arylsulphatase, an amylase, a cellulase, an inulase, a xylanase, a dextranase, a levanase, a poly-galacturonase, a glucosidase, a galactosidase, an invertase, a peptidase, an asparaginase, a glutaminase, an amidase, a urease, an aspartate decarboxylase, a glutamate decarboxylase an aromatic amino acid decarboxylase, or any combination thereof. An enzyme within a working fluid may comprise nitrogenase, 1-aminocyclopropane-1-carboxylate deaminase (e.g., ACC-deaminase), quinoprotein glucose dehydrogenase (e.g., PQQ or quinone), gluconate 2-dehydrogenase, cellulase, endo-1,3 (4)-β-glucanase, pectin lyase, or any combination thereof. The working fluid within each reactor may comprise different concentrations of enzymes. The working fluid within each reactor may comprise a different average abundance of an enzyme. An enzyme may be present at an average abundance between 0.001% to 1%. An enzyme may be present at an average abundance of less than 0.001%. An enzyme may be present at an average abundance of greater than 1%. In some embodiments, an enzyme may be present at an average abundance of at least about 0.0001%, at least about 0.001%, at least about 0.01%, at least about 0.02%, at least about 0.03%, at least about 0.04%, at least about 0.05%, at least about 0.06%, at least about 0.07%, at least about 0.08%, at least about 0.09%, at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, or at least about 5%. In some embodiments, an enzyme may be present at an average abundance of at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, at most about 0.09%, at most about 0.08%, at most about 0.07%, at most about 0.06%, at most about 0.05%, at most about 0.04%, at most about 0.03%, at most about 0.02%, at most about 0.01%, at most about 0.001%, or at most about 0.0001%. In some embodiments, an enzyme may be present at an average abundance of between about 0.0001% and about 0.001%, about 0.0001% and about 0.01%, about 0.0001% and about 0.01%, about 0.0001% and about 0.02%, about 0.0001% and about 0.03%, about 0.0001% and about 0.05%, about 0.0001% and about 0.06%, about 0.0001% and about 0.07%, about 0.0001% and about 0.08%, about 0.0001% and about 0.09%, about 0.0001% and about 0.1%, 0.0001% and about 0.2%, 0.0001% and about 0.3%, 0.0001% and about 0.4%, 0.0001% and about 0.5%, 0.0001% and about 0.6%, 0.0001% and about 0.7%, 0.0001% and about 0.8%, 0.0001% and about 0.9%, 0.0001% and about 1%, 0.0001% and about 2%, 0.0001% and about 3%, 0.0001% and about 4%, 0.0001% and about 5%, about 0.001% and about 0.01%, about 0.001% and about 0.02%, about 0.001% and about 0.03%, about 0.001% and about 0.05%, about 0.001% and about 0.06%, about 0.001% and about 0.07%, about 0.001% and about 0.08%, about 0.001% and about 0.09%, about 0.001% and about 0.1%, 0.001% and about 0.2%, 0.001% and about 0.3%, 0.001% and about 0.4%, 0.001% and about 0.5%, 0.001% and about 0.6%, 0.001% and about 0.7%, 0.001% and about 0.8%, 0.001% and about 0.9%, 0.001% and about 1%, 0.001% and about 2%, 0.001% and about 3%, 0.001% and about 4%, 0.001% and about 5%, about 0.01% and about 0.02%, about 0.01% and about 0.03%, about 0.01% and about 0.05%, about 0.01% and about 0.06%, about 0.01% and about 0.07%, about 0.01% and about 0.08%, about 0.01% and about 0.09%, about 0.01% and about 0.1%, 0.01% and about 0.2%, 0.01% and about 0.3%, 0.01% and about 0.4%, 0.01% and about 0.5%, 0.01% and about 0.6%, 0.01% and about 0.7%, 0.01% and about 0.8%, 0.01% and about 0.9%, 0.01% and about 1%, 0.01% and about 2%, 0.01% and about 3%, 0.01% and about 4%, 0.01% and about 5%, about 0.02% and about 0.03%, about 0.02% and about 0.05%, about 0.02% and about 0.06%, about 0.02% and about 0.07%, about 0.02% and about 0.08%, about 0.02% and about 0.09%, about 0.02% and about 0.1%, 0.02% and about 0.2%, 0.02% and about 0.3%, 0.02% and about 0.4%, 0.02% and about 0.5%, 0.02% and about 0.6%, 0.02% and about 0.7%, 0.02% and about 0.8%, 0.02% and about 0.9%, 0.02% and about 1%, 0.02% and about 2%, 0.02% and about 3%, 0.02% and about 4%, 0.02% and about 5%, about 0.03% and about 0.05%, about 0.03% and about 0.06%, about 0.03% and about 0.07%, about 0.03% and about 0.08%, about 0.03% and about 0.09%, about 0.03% and about 0.1%, 0.03% and about 0.2%, 0.03% and about 0.3%, 0.03% and about 0.4%, 0.03% and about 0.5%, 0.03% and about 0.6%, 0.03% and about 0.7%, 0.03% and about 0.8%, 0.03% and about 0.9%, 0.03% and about 1%, 0.03% and about 2%, 0.03% and about 3%, 0.03% and about 4%, 0.05% and about 5%, about 0.05% and about 0.06%, about 0.05% and about 0.07%, about 0.05% and about 0.08%, about 0.05% and about 0.09%, about 0.05% and about 0.1%, 0.05% and about 0.2%, 0.05% and about 0.3%, 0.05% and about 0.4%, 0.05% and about 0.5%, 0.05% and about 0.6%, 0.05% and about 0.7%, 0.05% and about 0.8%, 0.05% and about 0.9%, 0.05% and about 1%, 0.05% and about 2%, 0.05% and about 3%, 0.05% and about 4%, 0.05% and about 5%, about 0.06% and about 0.07%, about 0.06% and about 0.08%, about 0.06% and about 0.09%, about 0.06% and about 0.1%, 0.06% and about 0.2%, 0.06% and about 0.3%, 0.06% and about 0.4%, 0.06% and about 0.5%, 0.06% and about 0.6%, 0.06% and about 0.7%, 0.06% and about 0.8%, 0.06% and about 0.9%, 0.06% and about 1%, 0.06% and about 2%, 0.06% and about 3%, 0.06% and about 4%, 0.06% and about 5%, about 0.07% and about 0.08%, about 0.07% and about 0.09%, about 0.07% and about 0.1%, 0.07% and about 0.2%, 0.07% and about 0.3%, 0.07% and about 0.4%, 0.07% and about 0.5%, 0.07% and about 0.6%, 0.07% and about 0.7%, 0.07% and about 0.8%, 0.07% and about 0.9%, 0.07% and about 1%, 0.07% and about 2%, 0.07% and about 3%, 0.07% and about 4%, 0.07% and about 5%, about 0.08% and about 0.09%, about 0.08% and about 0.1%, 0.08% and about 0.2%, 0.08% and about 0.3%, 0.08% and about 0.4%, 0.08% and about 0.5%, 0.08% and about 0.6%, 0.08% and about 0.7%, 0.08% and about 0.8%, 0.08% and about 0.9%, 0.08% and about 1%, 0.08% and about 2%, 0.08% and about 3%, 0.08% and about 4%, 0.08% and about 5%, about 0.09% and about 0.1%, 0.09% and about 0.2%, 0.09% and about 0.3%, 0.09% and about 0.4%, 0.09% and about 0.5%, 0.09% and about 0.6%, 0.09% and about 0.7%, 0.09% and about 0.8%, 0.09% and about 0.9%, 0.09% and about 1%, 0.09% and about 2%, 0.09% and about 3%, 0.09% and about 4%, 0.09% and about 5%, 0.1% and about 0.2%, 0.1% and about 0.3%, 0.1% and about 0.4%, 0.1% and about 0.5%, 0.1% and about 0.6%, 0.1% and about 0.7%, 0.1% and about 0.8%, 0.1% and about 0.9%, 0.1% and about 1%, 0.1% and about 2%, 0.1% and about 3%, 0.1% and about 4%, 0.1% and about 5%, 0.2% and about 0.3%, 0.2% and about 0.4%, 0.2% and about 0.5%, 0.2% and about 0.6%, 0.2% and about 0.7%, 0.2% and about 0.8%, 0.2% and about 0.9%, 0.2% and about 1%, 0.2% and about 2%, 0.2% and about 3%, 0.2% and about 4%, 0.2% and about 5%, 0.3% and about 0.4%, 0.3% and about 0.5%, 0.3% and about 0.6%, 0.3% and about 0.7%, 0.3% and about 0.8%, 0.3% and about 0.9%, 0.3% and about 1%, 0.3% and about 2%, 0.3% and about 3%, 0.3% and about 4%, 0.3% and about 5%, 0.4% and about 0.5%, 0.4% and about 0.6%, 0.4% and about 0.7%, 0.4% and about 0.8%, 0.4% and about 0.9%, 0.4% and about 1%, 0.4% and about 2%, 0.4% and about 3%, 0.4% and about 4%, 0.4% and about 5%, 0.5% and about 0.6%, 0.5% and about 0.7%, 0.5% and about 0.8%, 0.5% and about 0.9%, 0.5% and about 1%, 0.5% and about 2%, 0.5% and about 3%, 0.5% and about 4%, 0.5% and about 5%, 0.6% and about 0.7%, 0.6% and about 0.8%, 0.6% and about 0.9%, 0.6% and about 1%, 0.6% and about 2%, 0.6% and about 3%, 0.6% and about 4%, 0.6% and about 5%, 0.7% and about 0.8%, 0.7% and about 0.9%, 0.7% and about 1%, 0.7% and about 2%, 0.7% and about 3%, 0.7% and about 4%, 0.7% and about 5%, 0.8% and about 0.9%, 0.8% and about 1%, 0.8% and about 2%, 0.8% and about 3%, 0.8% and about 4%, 0.8% and about 5%, 0.9% and about 1%, 0.9% and about 2%, 0.9% and about 3%, 0.9% and about 4%, 0.9% and about 5%, 1% and about 2%, 1% and about 3%, 1% and about 4%, 1% and about 5%, 2% and about 3%, 2% and about 4%, 2% and about 5%, 3% and about 4%, 3% and about 5%, or 4% and about 5%.

The working fluid within each reactor may comprise enzymes with different enzymatic activity. Enzymatic activity may include, but is not limited to, nitrogen fixation, ammonia production, phosphate solubilization, zinc solubilization, and/or cell wall lysing.

A working fluid may comprise different digestion products from working fluid within other reactors of the system. In some embodiments, a working fluid may comprise digestion products from a feedstock (e.g., aqueous organic feedstock) and microbial consortium at least partially derived from a previous working fluid.

The pH of a working fluid within each reactor may be different from working fluid in other reactors. The pH of a working fluid within each reactor may be the same. The pH of a working fluid may be less than 6. The pH of a working fluid may be greater than 6. The pH of a working fluid may be in a range from 2 to 11. The pH of a working fluid may be at least about 2, at least about 2.5, at least about 3, at least about 3.5, at least about 4, at least about 4.5, at least about 5, at least about 5.5, at least about 6, at least about 6.5, at least about 7, at least about 7.5, at least about 8, at least about 8.5, at least about 9, at least about 9.5, at least about 10, at least about 10.5, or at least about 11. The pH of a working fluid may be at most about 11, at most about 10.5, at most about 10, at most about 9.5, at most about 9, at most about 8.5, at most about 8, at most about 7.5, at most about 7, at most about 6.5, at most about 6, at most about 5.5, at most about 5, at most about 4.5, at most about 4, at most about 3.5, at most about 3, at most about 2.5, or at most about 2.

The system 100 may comprise a pH sensor 191, pH controller, and/or a buffer addition system 190 to detect and/or control the pH in the reactor so that the pH is maintained at a threshold (e.g., pH 7). The buffer addition system 190 may be automatic. For example, if the pH of the working fluid in the first reactor is below the threshold value, an automatic base (e.g., 3M of NaOH) may be added to the first reactor until the pH reaches the threshold value (e.g., at least about 7).

In some cases, fluid (e.g., working fluid) can flow in a hydraulically balanced manner. The clarifier container 120 produces biostimulant products or digestion products (e.g., base products) 150. In some embodiments, a pH in a reactor of bioreactor system 100 may be between 4.0-9.0. In some embodiments, the pH in the reactor may be at least about 3.0, at least about 3.5, at least about 4.0, at least about 4.5, at least about 5.0, at least about 5.5, at least about 6.0, at least about 6.5, at least about 7.0, at least about 7.5, at least about 8.0, at least about 8.5, at least about 9.0, at least about 9.5, or at least about 10.0.

In the first reactor, the working fluid may be agitated at a rate that allows heavier or undigested solids to settle to the bottom. An outlet at the top of the first reactors may allow the fluid to flow into the second reactor. An outlet at the bottom of the first reactor may transfer the settled solids back into a reactor tank. Each of the reactors in the series of reactors may have submerged scaffolding that provide a surface for biofilm growth. The scaffolding can be referred to as “fixed media substrates”. The flow of fluid from reactor to reactor may comprise a plug flow model, in which particles of an input fluid have the same velocity and direction of motion. In some embodiments, the flow of fluid in the bioreactor system is driven by gravity. In some embodiments, the flow of fluid in the bioreactor system is driven by a pump. The outflow from the top of the last reactor, may be used to create a product. Products of the methods and systems as described herein can be biostimulants.

Fluid (e.g., working fluid) from an outflow port (e.g., a product outflow port) of the first reactor 161 can flow into the second reactor 112 continuously. Fluid from an outflow port (e.g., a product outflow port) of the second reactor 112 can flow into the third reactor 113 continuously. Fluid from an outflow port (e.g., a product outflow port) of the third reactor 113 can flow into the clarifier container 120 continuously.

The outflow port may be positioned on the top, middle, and/or bottom of a reactor. Additionally, each reactor or clarifier container may comprise another outflow port for reintroducing fluid back into the same reactor or clarifier container, and may be pumped back to just below the surface of the same reactor to maintain homogeneous conditions within the working solutions. In some cases, the working fluid from each reactor is recirculated within each reactor from the bottom of the reactor back to just below the surface of the working solution to maintain a homogeneous environment for fermentation. For example, fluid from the first reactor 111 may be reintroduced back into the first reactor 111. Biosolids (e.g., floc) may be generated through the process. In some embodiments, floc can be a flocculated mass of microorganisms, extracellular polymeric substance (EPS) and adsorbed organic and inorganic material. A flocculated mass can comprise an aggregated mass of microorganisms, extracellular polymeric substance (EPS) and adsorbed organic and inorganic material. In some cases, the floc may be returned manually back to a first reactor 111. In some cases, the floc may be returned to any container of the bioreactor system 100.

In some embodiments, the bioreactor system comprises a clarifier container or clarifier tank (CLF) 120. In some embodiments, the clarifier container is the final container in a bioreactor system. In some embodiments, the clarifier container is not the final container in a bioreactor system. The clarifier may comprise a single in-flow port and a single outflow port. The clarifier may comprise a single in-flow port and two or more outflow ports. In some embodiments, the clarifier comprises floc-folding wipes which rotate and release microbes that have been immobilized in the floc without introducing solids in the supernatant. The floc-folding wipers may move a working fluid in the clarifier to re-suspend microbes within the working fluid. In some embodiments, the microbes and/or an amount of target isolate strain may be re-suspended in the solution in the clarifier and transferred to the supernatant. In some cases, the clarifier container 120 may comprise an outflow port (e.g., a product outflow port) for reintroducing fluid back to the first reactor 111. In some embodiments, the clarifier further comprises a flow line to return floc to the first reactor. The flow line may comprise a conduit from the clarifier to a container or combination of containers of the bioreactor system (e.g., a first container, a second container, a third container, a fourth container, a fifth container, a sixth container, or any combination thereof). The clarifier may return flow to any container (e.g., a first container, a second container, a third container, a fourth container, a fifth container, a sixth container, or any combination thereof) of a bioreactor system to provide a recirculation of working fluid. The working fluid recirculated from the clarifier may comprise a different microbial community (e.g., different amounts of microbes, different types of microbes) than a working fluid of another container in the bioreactor system (e.g., a first working fluid, a second working fluid, a third working fluid, a fourth working fluid, a fifth working fluid, and/or a sixth working fluid). Without wishing to be bound by theory, the recirculation of flow from the clarifier to a container of the bioreactor system may help enrich a microbial community of a microbial consortium of a bioreactor system by providing working fluid from the clarifier to a different point (e.g., container) of the system. The recirculated working fluid may comprise organic materials, microbes of a microbial consortium, a target isolate, metabolites, or any combination thereof.

In the clarifier, a floc portion of a working fluid (e.g., a clarifier working fluid) may separate from a supernatant portion of a working fluid. The floc-folding wipers of the clarifier may help in separating the working fluid of the clarifier. In some embodiments, the separating may comprise gravity separation. The floc may settle on the bottom of the clarifier and the supernatant may be collected.

In some embodiments, the supernatant from the clarifier container may be continuously collected, and a portion of the floc at the bottom of the clarifier can be returned to the first reactor at a rate of recirculation of at least about 0.5 L/day, at least about 1.0 L/day, at least about 1.5 L/day, at least about 2.0 L/day, at least about 2.5 L/day, at least about 3.0 L/day, at least about 3.5 L/day, at least about 4.0 L/day, at least about 4.1 L/day, at least about 4.2 L/day, at least about 4.3 L/day, at least about 4.4 L/day, at least about 4.5 L/day, at least about 4.51 L/day, at least about 4.52 L/day, at least about 4.53 L/day, at least about 4.54 L/day, at least about 4.55 L/day, at least about 4.56 L/day, at least about 4.57 L/day, at least about 4.58 L/day, at least about 4.59 L/day, at least about 4.6 L/day, at least about 4.7 L/day, at least about 4.8 L/day, at least about 4.9 L/day, at least about 5.0 L/day, at least about 6.0 L/day, at least about 7.0 L/day, at least about 8.0 L/day, at least about 9.0 L/day, or at least about 10.0 L/day. In some embodiments, the supernatant from the clarifier container may be continuously collected, and a portion of the floc at the bottom of the clarifier can be returned to the first reactor at a rate of recirculation of at least about 10 L/day, at least about 50 L/day, at least about 100 L/day, at least about 200 L/day, at least about 300 L/day, at least about 400 L/day, at least about 500 L/day, at least about 750 L/day, at least about 1000 L/day, at least about 2500 L/day, at least about 5000 L/day, or greater than about 5000 L/day. In some embodiments, the supernatant from the clarifier container may be continuously collected, and a portion of the floc at the bottom of the clarifier can be returned to the first reactor at a rate of recirculation of at most about 5000 L/day, at most about 2500 L/day, at most about 1000 L/day, at most about 750 L/day, at most about 500 L/day, at most about 400 L/day, at most about 300 L/day, at most about 200 L/day, at most about 100 L/day, at most about 50 L/day, at most about 10 L/day, or less than about 10 L/day.

In some embodiments, the supernatant from the clarifier container may be continuously collected, and a portion of the floc at the bottom of the clarifier can be returned to the first reactor at a rate of recirculation of at most about 10.0 L/day, at most about 9.0 L/day, at most about 8.0 L/day, at most about 7.0 L/day, at most about 6.0 L/day, at most about 5.5 L/day, at most about 5.0 L/day, at most about 4.9 L/day, at most about 4.8 L/day, at most about 4.7 L/day, at most about 4.6 L/day, at most about 4.59 L/day, at most about 4.58 L/day, at most about 4.57 L/day, at most about 4.56 L/day, at most about 4.55 L/day, at most about 4.54 L/day, at most about 4.53 L/day, at most about 4.52 L/day, at most about 4.51 L/day, at most about 4.5 L/day, at most about 4.4 L/day, at most about 4.3 L/day, at most about 4.2 L/day, at most about 4.1 L/day, at most about 4.0 L/day, at most about 3.5 L/day, at most about 3.0 L/day, at most about 2.5 L/day, at most about 2.0 L/day, at most about 1.5 L/day, at most about 1.0 L/day, or at most about 0.5 L/day.

In some embodiments, the supernatant from the clarifier container may be continuously collected, and a portion of the floc at the bottom of the clarifier can be returned to the first reactor at a rate of recirculation between about 1 L/day to about 1,000 L/day. In some embodiments, the supernatant from the clarifier container may be continuously collected, and a portion of the floc at the bottom of the clarifier can be returned to the first reactor at a rate of recirculation between at most about 1,000 L/day. In some embodiments, the supernatant from the clarifier container may be continuously collected, and a portion of the floc at the bottom of the clarifier can be returned to the first reactor at a rate of recirculation between about 1 L/day to about 5 L/day, about 1 L/day to about 10 L/day, about 1 L/day to about 25 L/day, about 1 L/day to about 50 L/day, about 1 L/day to about 100 L/day, about 1 L/day to about 200 L/day, about 1 L/day to about 300 L/day, about 1 L/day to about 400 L/day, about 1 L/day to about 500 L/day, about 1 L/day to about 750 L/day, about 1 L/day to about 1,000 L/day, about 5 L/day to about 10 L/day, about 5 L/day to about 25 L/day, about 5 L/day to about 50 L/day, about 5 L/day to about 100 L/day, about 5 L/day to about 200 L/day, about 5 L/day to about 300 L/day, about 5 L/day to about 400 L/day, about 5 L/day to about 500 L/day, about 5 L/day to about 750 L/day, about 5 L/day to about 1,000 L/day, about 10 L/day to about 25 L/day, about 10 L/day to about 50 L/day, about 10 L/day to about 100 L/day, about 10 L/day to about 200 L/day, about 10 L/day to about 300 L/day, about 10 L/day to about 400 L/day, about 10 L/day to about 500 L/day, about 10 L/day to about 750 L/day, about 10 L/day to about 1,000 L/day, about 25 L/day to about 50 L/day, about 25 L/day to about 100 L/day, about 25 L/day to about 200 L/day, about 25 L/day to about 300 L/day, about 25 L/day to about 400 L/day, about 25 L/day to about 500 L/day, about 25 L/day to about 750 L/day, about 25 L/day to about 1,000 L/day, about 50 L/day to about 100 L/day, about 50 L/day to about 200 L/day, about 50 L/day to about 300 L/day, about 50 L/day to about 400 L/day, about 50 L/day to about 500 L/day, about 50 L/day to about 750 L/day, about 50 L/day to about 1,000 L/day, about 100 L/day to about 200 L/day, about 100 L/day to about 300 L/day, about 100 L/day to about 400 L/day, about 100 L/day to about 500 L/day, about 100 L/day to about 750 L/day, about 100 L/day to about 1,000 L/day, about 200 L/day to about 300 L/day, about 200 L/day to about 400 L/day, about 200 L/day to about 500 L/day, about 200 L/day to about 750 L/day, about 200 L/day to about 1,000 L/day, about 300 L/day to about 400 L/day, about 300 L/day to about 500 L/day, about 300 L/day to about 750 L/day, about 300 L/day to about 1,000 L/day, about 400 L/day to about 500 L/day, about 400 L/day to about 750 L/day, about 400 L/day to about 1,000 L/day, about 500 L/day to about 750 L/day, about 500 L/day to about 1,000 L/day, or about 750 L/day to about 1,000 L/day.

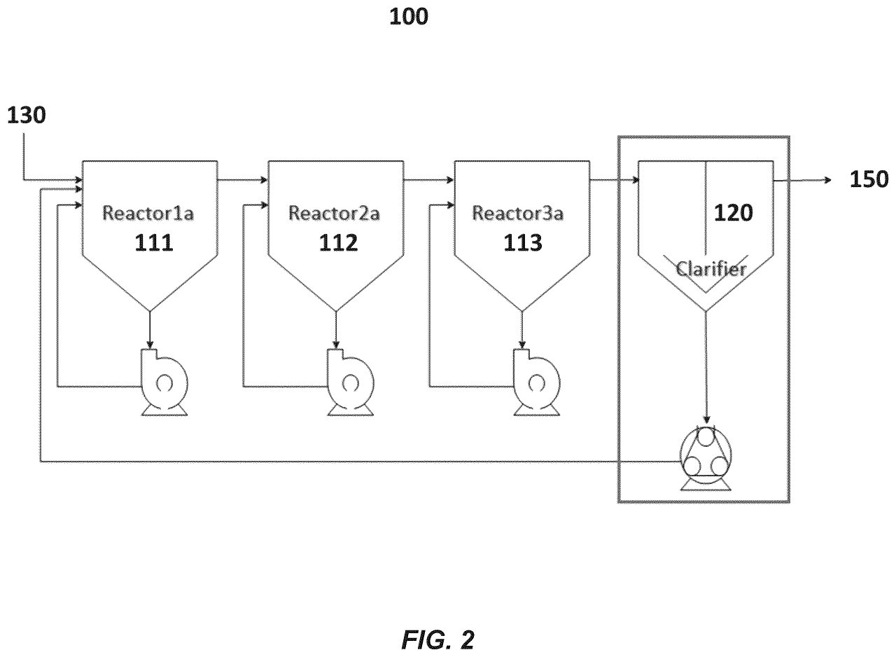

In some embodiments, the clarifier may comprise a floc flight system (e.g., floc-folding flights) as shown in FIG. 2 that can improve the concentration of the added specific isolate(s) in the base product. With the addition of the floc flights in the clarifier, the concentration of a target isolate in the base product may be maintained at a higher concentration for a longer period. In the clarifier, floc may settle or become immobilized in the bottom of the clarifier. Slow, floc-folding flights may release immobilized amount(s) of the target isolate strain in the floc. The released target isolate can then be returned (e.g., be reinoculated) into the bioreactor system. The floc-folding flights may re-suspend an amount of target isolate into the supernatant (e.g., base product). In some embodiments, the floc may return back to the system at a concentration of least about a 0.1% v/v rate per day, at least about a 0.5% v/v rate per day, at least about a 1.0% v/v rate per day, at least about a 1.1% v/v rate per day, at least about a 1.2% v/v rate per day, at least about a 1.3% v/v rate per day, at least about a 1.4% v/v rate per day, at least about a 1.5% v/v rate per day, at least about a 1.6% v/v rate per day, at least about a 1.7% v/v rate per day, at least about a 1.8% v/v rate per day, at least about a 1.9% v/v rate per day, at least about a 2.0% v/v rate per day, at least about a 2.1% v/v rate per day, at least about a 2.2% v/v rate per day, at least about a 2.3% v/v rate per day, at least about a 2.4% v/v rate per day, at least about a 2.5% v/v rate per day, at least about a 2.6% v/v rate per day, at least about a 2.7% v/v rate per day, at least about a 2.8% v/v rate per day, at least about a 2.9% v/v rate per day, at least about a 3.0% v/v rate per day, at least about a 4.0% v/v rate per day, at least about a 5.0% v/v rate per day, at least about a 6.0% v/v rate per day, at least about a 7.0% v/v rate per day, at least about a 8.0% v/v rate per day, at least about a 9.0% v/v rate per day, or at least about a 10.0% v/v rate per day.

In some embodiments, the floc may return back to the system at most about a concentration of 10.0% v/v rate per day, 9.0% v/v rate per day, 8.0% v/v rate per day, 7.0% v/v rate per day, 6.0% v/v rate per day, 5.0% v/v rate per day, 4.0% v/v rate per day, 3.0% v/v rate per day, 2.9% v/v rate per day, 2.8% v/v rate per day, 2.7% v/v rate per day, 2.6% v/v rate per day, 2.5% v/v rate per day, 2.4% v/v rate per day, 2.3% v/v rate per day, 2.2% v/v rate per day, 2.1% v/v rate per day, 2.0% v/v rate per day, 1.9% v/v rate per day, 1.8% v/v rate per day, 1.7% v/v rate per day, 1.6% v/v rate per day, 1.5% v/v rate per day, 1.4% v/v rate per day, 1.3% v/v rate per day, 1.2% v/v rate per day, 1.1% v/v rate per day, 1.0% v/v rate per day, 0.5% v/v rate per day, or 0.1% v/v rate per day.

In some embodiments, the floc may return back to the system at a concentration of about 0.1% v/v rate per day to about 15% v/v rate per day. In some embodiments, the floc may return back to the system at a concentration of about 0.1% v/v rate per day to about 0.5% v/v rate per day, about 0.1% v/v rate per day to about 1% v/v rate per day, about 0.1% v/v rate per day to about 1.5% v/v rate per day, about 0.1% v/v rate per day to about 2% v/v rate per day, about 0.1% v/v rate per day to about 3% v/v rate per day, about 0.1% v/v rate per day to about 4% v/v rate per day, about 0.1% v/v rate per day to about 5% v/v rate per day, about 0.1% v/v rate per day to about 7% v/v rate per day, about 0.1% v/v rate per day to about 10% v/v rate per day, about 0.1% v/v rate per day to about 12% v/v rate per day, about 0.1% v/v rate per day to about 15% v/v rate per day, about 0.5% v/v rate per day to about 1% v/v rate per day, about 0.5% v/v rate per day to about 1.5% v/v rate per day, about 0.5% v/v rate per day to about 2% v/v rate per day, about 0.5% v/v rate per day to about 3% v/v rate per day, about 0.5% v/v rate per day to about 4% v/v rate per day, about 0.5% v/v rate per day to about 5% v/v rate per day, about 0.5% v/v rate per day to about 7% v/v rate per day, about 0.5% v/v rate per day to about 10% v/v rate per day, about 0.5% v/v rate per day to about 12% v/v rate per day, about 0.5% v/v rate per day to about 15% v/v rate per day, about 1% v/v rate per day to about 1.5% v/v rate per day, about 1% v/v rate per day to about 2% v/v rate per day, about 1% v/v rate per day to about 3% v/v rate per day, about 1% v/v rate per day to about 4% v/v rate per day, about 1% v/v rate per day to about 5% v/v rate per day, about 1% v/v rate per day to about 7% v/v rate per day, about 1% v/v rate per day to about 10% v/v rate per day, about 1% v/v rate per day to about 12% v/v rate per day, about 1% v/v rate per day to about 15% v/v rate per day, about 1.5% v/v rate per day to about 2% v/v rate per day, about 1.5% v/v rate per day to about 3% v/v rate per day, about 1.5% v/v rate per day to about 4% v/v rate per day, about 1.5% v/v rate per day to about 5% v/v rate per day, about 1.5% v/v rate per day to about 7% v/v rate per day, about 1.5% v/v rate per day to about 10% v/v rate per day, about 1.5% v/v rate per day to about 12% v/v rate per day, about 1.5% v/v rate per day to about 15% v/v rate per day, about 2% v/v rate per day to about 3% v/v rate per day, about 2% v/v rate per day to about 4% v/v rate per day, about 2% v/v rate per day to about 5% v/v rate per day, about 2% v/v rate per day to about 7% v/v rate per day, about 2% v/v rate per day to about 10% v/v rate per day, about 2% v/v rate per day to about 12% v/v rate per day, about 2% v/v rate per day to about 15% v/v rate per day, about 3% v/v rate per day to about 4% v/v rate per day, about 3% v/v rate per day to about 5% v/v rate per day, about 3% v/v rate per day to about 7% v/v rate per day, about 3% v/v rate per day to about 10% v/v rate per day, about 3% v/v rate per day to about 12% v/v rate per day, about 3% v/v rate per day to about 15% v/v rate per day, about 4% v/v rate per day to about 5% v/v rate per day, about 4% v/v rate per day to about 7% v/v rate per day, about 4% v/v rate per day to about 10% v/v rate per day, about 4% v/v rate per day to about 12% v/v rate per day, about 4% v/v rate per day to about 15% v/v rate per day, about 5% v/v rate per day to about 7% v/v rate per day, about 5% v/v rate per day to about 10% v/v rate per day, about 5% v/v rate per day to about 12% v/v rate per day, about 5% v/v rate per day to about 15% v/v rate per day, about 7% v/v rate per day to about 10% v/v rate per day, about 7% v/v rate per day to about 12% v/v rate per day, about 7% v/v rate per day to about 15% v/v rate per day, about 10% v/v rate per day to about 12% v/v rate per day, about 10% v/v rate per day to about 15% v/v rate per day, or about 12% v/v rate per day to about 15% v/v rate per day.

In some embodiments, the floc may return back to the system at a concentration of about 0.1% v/v rate per day to about 3% v/v rate per day. In some embodiments, the floc may return back to the system at a concentration of about 0.1% v/v rate per day to about 0.5% v/v rate per day, about 0.1% v/v rate per day to about 1% v/v rate per day, about 0.1% v/v rate per day to about 1.25% v/v rate per day, about 0.1% v/v rate per day to about 1.5% v/v rate per day, about 0.1% v/v rate per day to about 1.75% v/v rate per day, about 0.1% v/v rate per day to about 2% v/v rate per day, about 0.1% v/v rate per day to about 2.25% v/v rate per day, about 0.1% v/v rate per day to about 2.5% v/v rate per day, about 0.1% v/v rate per day to about 2.75% v/v rate per day, about 0.1% v/v rate per day to about 3% v/v rate per day, about 0.5% v/v rate per day to about 1% v/v rate per day, about 0.5% v/v rate per day to about 1.25% v/v rate per day, about 0.5% v/v rate per day to about 1.5% v/v rate per day, about 0.5% v/v rate per day to about 1.75% v/v rate per day, about 0.5% v/v rate per day to about 2% v/v rate per day, about 0.5% v/v rate per day to about 2.25% v/v rate per day, about 0.5% v/v rate per day to about 2.5% v/v rate per day, about 0.5% v/v rate per day to about 2.75% v/v rate per day, about 0.5% v/v rate per day to about 3% v/v rate per day, about 1% v/v rate per day to about 1.25% v/v rate per day, about 1% v/v rate per day to about 1.5% v/v rate per day, about 1% v/v rate per day to about 1.75% v/v rate per day, about 1% v/v rate per day to about 2% v/v rate per day, about 1% v/v rate per day to about 2.25% v/v rate per day, about 1% v/v rate per day to about 2.5% v/v rate per day, about 1% v/v rate per day to about 2.75% v/v rate per day, about 1% v/v rate per day to about 3% v/v rate per day, about 1.25% v/v rate per day to about 1.5% v/v rate per day, about 1.25% v/v rate per day to about 1.75% v/v rate per day, about 1.25% v/v rate per day to about 2% v/v rate per day, about 1.25% v/v rate per day to about 2.25% v/v rate per day, about 1.25% v/v rate per day to about 2.5% v/v rate per day, about 1.25% v/v rate per day to about 2.75% v/v rate per day, about 1.25% v/v rate per day to about 3% v/v rate per day, about 1.5% v/v rate per day to about 1.75% v/v rate per day, about 1.5% v/v rate per day to about 2% v/v rate per day, about 1.5% v/v rate per day to about 2.25% v/v rate per day, about 1.5% v/v rate per day to about 2.5% v/v rate per day, about 1.5% v/v rate per day to about 2.75% v/v rate per day, about 1.5% v/v rate per day to about 3% v/v rate per day, about 1.75% v/v rate per day to about 2% v/v rate per day, about 1.75% v/v rate per day to about 2.25% v/v rate per day, about 1.75% v/v rate per day to about 2.5% v/v rate per day, about 1.75% v/v rate per day to about 2.75% v/v rate per day, about 1.75% v/v rate per day to about 3% v/v rate per day, about 2% v/v rate per day to about 2.25% v/v rate per day, about 2% v/v rate per day to about 2.5% v/v rate per day, about 2% v/v rate per day to about 2.75% v/v rate per day, about 2% v/v rate per day to about 3% v/v rate per day, about 2.25% v/v rate per day to about 2.5% v/v rate per day, about 2.25% v/v rate per day to about 2.75% v/v rate per day, about 2.25% v/v rate per day to about 3% v/v rate per day, about 2.5% v/v rate per day to about 2.75% v/v rate per day, about 2.5% v/v rate per day to about 3% v/v rate per day, or about 2.75% v/v rate per day to about 3% v/v rate per day.

In some embodiments, as solids accumulate over time in the clarifier container, a range of at least about 20-25% solids v/v may be maintained in the bioreactor system. In some embodiments, additional floc may be harvested from the bioreactor system and removed. In some embodiments, at least about 5% solids v/v, at least about 10% solids v/v, at least about 15% solids v/v, at least about 16% solids v/v, at least about 17% solids v/v, at least about 18% solids v/v, at least about 19% solids v/v, at least about 20% solids v/v, at least about 21% solids v/v, at least about 22% solids v/v, at least about 23% solids v/v, at least about 24% solids v/v, at least about 25% solids v/v, at least about 26% solids v/v, at least about 27% solids v/v, at least about 28% solids v/v, at least about 29% solids v/v, at least about 30% solids v/v, at least about 35% solids v/v, at least about 40% solids v/v, at least about 45% solids v/v, or at least about 50% solids v/v may be maintained in the bioreactor system.

In some embodiments, at most about 50% solids v/v, at most about 45% solids v/v, at most about 40% solids v/v, at most about 35% solids v/v, at most about 30% solids v/v, at most about 29% solids v/v, at most about 28% solids v/v, at most about 27% solids v/v, at most about 26% solids v/v, at most about 25% solids v/v, at most about 24% solids v/v, at most about 23% solids v/v, at most about 22% solids v/v, at most about 21% solids v/v, at most about 20% solids v/v, at most about 19% solids v/v, at most about 18% solids v/v, at most about 17% solids v/v, at most about 16% solids v/v, at most about 15% solids v/v, at most about 14% solids v/v, at most about 13% solids v/v, at most about 12% solids v/v, at most about 11% solids v/v, at most about 10% solids v/v, or at most about 5% solids v/v may be maintained in the bioreactor system.

In some embodiments, about 0.1% solids v/v to about 60% solids v/v may be maintained in the bioreactor system. In some embodiments, about 0.1% solids v/v to about 1% solids v/v, about 0.1% solids v/v to about 5% solids v/v, about 0.1% solids v/v to about 10% solids v/v, about 0.1% solids v/v to about 15% solids v/v, about 0.1% solids v/v to about 20% solids v/v, about 0.1% solids v/v to about 25% solids v/v, about 0.1% solids v/v to about 30% solids v/v, about 0.1% solids v/v to about 35% solids v/v, about 0.1% solids v/v to about 40% solids v/v, about 0.1% solids v/v to about 50% solids v/v, about 0.1% solids v/v to about 60% solids v/v, about 1% solids v/v to about 5% solids v/v, about 1% solids v/v to about 10% solids v/v, about 1% solids v/v to about 15% solids v/v, about 1% solids v/v to about 20% solids v/v, about 1% solids v/v to about 25% solids v/v, about 1% solids v/v to about 30% solids v/v, about 1% solids v/v to about 35% solids v/v, about 1% solids v/v to about 40% solids v/v, about 1% solids v/v to about 50% solids v/v, about 1% solids v/v to about 60% solids v/v, about 5% solids v/v to about 10% solids v/v, about 5% solids v/v to about 15% solids v/v, about 5% solids v/v to about 20% solids v/v, about 5% solids v/v to about 25% solids v/v, about 5% solids v/v to about 30% solids v/v, about 5% solids v/v to about 35% solids v/v, about 5% solids v/v to about 40% solids v/v, about 5% solids v/v to about 50% solids v/v, about 5% solids v/v to about 60% solids v/v, about 10% solids v/v to about 15% solids v/v, about 10% solids v/v to about 20% solids v/v, about 10% solids v/v to about 25% solids v/v, about 10% solids v/v to about 30% solids v/v, about 10% solids v/v to about 35% solids v/v, about 10% solids v/v to about 40% solids v/v, about 10% solids v/v to about 50% solids v/v, about 10% solids v/v to about 60% solids v/v, about 15% solids v/v to about 20% solids v/v, about 15% solids v/v to about 25% solids v/v, about 15% solids v/v to about 30% solids v/v, about 15% solids v/v to about 35% solids v/v, about 15% solids v/v to about 40% solids v/v, about 15% solids v/v to about 50% solids v/v, about 15% solids v/v to about 60% solids v/v, about 20% solids v/v to about 25% solids v/v, about 20% solids v/v to about 30% solids v/v, about 20% solids v/v to about 35% solids v/v, about 20% solids v/v to about 40% solids v/v, about 20% solids v/v to about 50% solids v/v, about 20% solids v/v to about 60% solids v/v, about 25% solids v/v to about 30% solids v/v, about 25% solids v/v to about 35% solids v/v, about 25% solids v/v to about 40% solids v/v, about 25% solids v/v to about 50% solids v/v, about 25% solids v/v to about 60% solids v/v, about 30% solids v/v to about 35% solids v/v, about 30% solids v/v to about 40% solids v/v, about 30% solids v/v to about 50% solids v/v, about 30% solids v/v to about 60% solids v/v, about 35% solids v/v to about 40% solids v/v, about 35% solids v/v to about 50% solids v/v, about 35% solids v/v to about 60% solids v/v, about 40% solids v/v to about 50% solids v/v, about 40% solids v/v to about 60% solids v/v, or about 50% solids v/v to about 60% solids v/v.

In some embodiments, about 18% solids v/v to about 29% solids v/v. In some embodiments, about 18% solids v/v to about 19% solids v/v, about 18% solids v/v to about 20% solids v/v, about 18% solids v/v to about 21% solids v/v, about 18% solids v/v to about 22% solids v/v, about 18% solids v/v to about 23% solids v/v, about 18% solids v/v to about 24% solids v/v, about 18% solids v/v to about 25% solids v/v, about 18% solids v/v to about 26% solids v/v, about 18% solids v/v to about 27% solids v/v, about 18% solids v/v to about 28% solids v/v, about 18% solids v/v to about 29% solids v/v, about 19% solids v/v to about 20% solids v/v, about 19% solids v/v to about 21% solids v/v, about 19% solids v/v to about 22% solids v/v, about 19% solids v/v to about 23% solids v/v, about 19% solids v/v to about 24% solids v/v, about 19% solids v/v to about 25% solids v/v, about 19% solids v/v to about 26% solids v/v, about 19% solids v/v to about 27% solids v/v, about 19% solids v/v to about 28% solids v/v, about 19% solids v/v to about 29% solids v/v, about 20% solids v/v to about 21% solids v/v, about 20% solids v/v to about 22% solids v/v, about 20% solids v/v to about 23% solids v/v, about 20% solids v/v to about 24% solids v/v, about 20% solids v/v to about 25% solids v/v, about 20% solids v/v to about 26% solids v/v, about 20% solids v/v to about 27% solids v/v, about 20% solids v/v to about 28% solids v/v, about 20% solids v/v to about 29% solids v/v, about 21% solids v/v to about 22% solids v/v, about 21% solids v/v to about 23% solids v/v, about 21% solids v/v to about 24% solids v/v, about 21% solids v/v to about 25% solids v/v, about 21% solids v/v to about 26% solids v/v, about 21% solids v/v to about 27% solids v/v, about 21% solids v/v to about 28% solids v/v, about 21% solids v/v to about 29% solids v/v, about 22% solids v/v to about 23% solids v/v, about 22% solids v/v to about 24% solids v/v, about 22% solids v/v to about 25% solids v/v, about 22% solids v/v to about 26% solids v/v, about 22% solids v/v to about 27% solids v/v, about 22% solids v/v to about 28% solids v/v, about 22% solids v/v to about 29% solids v/v, about 23% solids v/v to about 24% solids v/v, about 23% solids v/v to about 25% solids v/v, about 23% solids v/v to about 26% solids v/v, about 23% solids v/v to about 27% solids v/v, about 23% solids v/v to about 28% solids v/v, about 23% solids v/v to about 29% solids v/v, about 24% solids v/v to about 25% solids v/v, about 24% solids v/v to about 26% solids v/v, about 24% solids v/v to about 27% solids v/v, about 24% solids v/v to about 28% solids v/v, about 24% solids v/v to about 29% solids v/v, about 25% solids v/v to about 26% solids v/v, about 25% solids v/v to about 27% solids v/v, about 25% solids v/v to about 28% solids v/v, about 25% solids v/v to about 29% solids v/v, about 26% solids v/v to about 27% solids v/v, about 26% solids v/v to about 28% solids v/v, about 26% solids v/v to about 29% solids v/v, about 27% solids v/v to about 28% solids v/v, about 27% solids v/v to about 29% solids v/v, or about 28% solids v/v to about 29% solids v/v may be maintained in the bioreactor system.

Inputs into bioreactor systems may comprise one or more of water, a microbial inoculum (e.g., an inoculum of a microbe or an inoculum of a microbial strain), nutrients (e.g., one or more sources of carbon, nitrogen, phosphorous, etc.), and a digestion substrate. In some embodiments, loading inputs into a reactor can comprise a carbon source, a nitrogen source, a flour, an isolate, or any combination thereof. In some embodiments, the loading inputs comprise recycled floc from the system. In some embodiments, the loading inputs comprise a whole broth (WB). The inoculum of a microbe as described herein may metabolize the carbon source. Metabolism of carbon by the inoculum of the microbe may comprise transfer of carbon-based moieties of the carbon source to substrates in the working fluid.

Digestion substrates an input stream into a bioreactor system may comprise, for example, organic materials that can be digested by microbes in the bioreactor system. Such organic materials may comprise, for example, manure, lignocellulosic material, wastewater biosolids, food waste, energy crops, yeast, guano, agricultural waste, algae, or any combination thereof. The manure may be chicken manure, cow manure, horse manure, sheep manure, alpaca manure, rabbit manure, and/or pig manure. In some embodiments, the manure is a mixture of one, two, three, or more manures. Digestion substrates input into bioreactor systems may have been subject to a partial digestion before being input into the system. Thus, the input into the system may comprise products of digestion of an original digestion substrate by microbes endogenous to the original digestion substrate, as well as digestible materials still present in the input. In some embodiments, digestion substrates in an input stream may comprise an inorganic substrate. The inorganic substrate may comprise, for example, sand, vermiculite, perlite, and/or pumice. In some embodiments, the inorganic substrates comprises a mineral. In some embodiments, the inorganic substrate comprises rock phosphate. In some embodiments, an input stream may comprise a source of zinc such as, for example, an insoluble form of zinc.

In some embodiments, the input composition to the bioreactor system 100 may comprise a carbon source 131. The carbon source may be glucose, malate, gluconic acid, lactose, sucrose, pyruvate, other simple sugars, or any combination thereof. In some cases, the carbon source may be added to the bioreactor system on a first day of a digestion process. In some cases, the carbon source may be added to the bioreactor system on a second day, third day, fourth day, or any day following a first day of a digestion process. The carbon source (e.g., glucose) may be added to the reactor to maintain a concentration range of 0.2%-3.0% w/v of carbon based on the total volume of the working fluid of the system. In some embodiments, the concentration of carbon that may be maintained can be based on the total concentration of all components of the working fluid in the at a point during the digestion process of the bioreactor system 100.

In some embodiments, the carbon source (e.g., glucose, malate) may be added to a reactor to maintain a concentration of carbon source in the working fluid of at least about 0.1%, at least about 0.25%, at least about 0.5%, at least about 0.75%, at least about 1.0%, at least about 1.25%, at least about 1.5%, at least about 1.75%, at least about 2.0%, at least about 2.5%, at least about 3.0%, at least about 5.0%, at least about 7.5%, or at least about 10.0% w/v. In some embodiments, the carbon source (e.g., glucose, malate) may be added to the first reactor to maintain a concentration of carbon source in the working fluid of at most about 10.0%, at most about 7.5%, at most about 5.0%, at most about 3.0%, at most about 2.5%, at most about 2.0%, at most about 1.75%, at most about 1.5%, at most about 1.25%, at most about 1.0%, at most about 0.75%, at most about 0.5%, at most about 0.25%, or at most about 0.1% w/v.

In some embodiments, the carbon source (e.g., glucose) may be added to a reactor to maintain a concentration of carbon source in the working fluid of about 0.1% w/v to about 5% w/v. In some embodiments, the carbon source (e.g., glucose) may be added to a reactor to maintain a concentration of carbon source in the working fluid of about 0.1% w/v to about 0.25% w/v, about 0.1% w/v to about 0.5% w/v, about 0.1% w/v to about 0.75% w/v, about 0.1% w/v to about 1% w/v, about 0.1% w/v to about 1.25% w/v, about 0.1% w/v to about 1.5% w/v, about 0.1% w/v to about 1.75% w/v, about 0.1% w/v to about 2% w/v, about 0.1% w/v to about 2.5% w/v, about 0.1% w/v to about 3% w/v, about 0.1% w/v to about 5% w/v, about 0.25% w/v to about 0.5% w/v, about 0.25% w/v to about 0.75% w/v, about 0.25% w/v to about 1% w/v, about 0.25% w/v to about 1.25% w/v, about 0.25% w/v to about 1.5% w/v, about 0.25% w/v to about 1.75% w/v, about 0.25% w/v to about 2% w/v, about 0.25% w/v to about 2.5% w/v, about 0.25% w/v to about 3% w/v, about 0.25% w/v to about 5% w/v, about 0.5% w/v to about 0.75% w/v, about 0.5% w/v to about 1% w/v, about 0.5% w/v to about 1.25% w/v, about 0.5% w/v to about 1.5% w/v, about 0.5% w/v to about 1.75% w/v, about 0.5% w/v to about 2% w/v, about 0.5% w/v to about 2.5% w/v, about 0.5% w/v to about 3% w/v, about 0.5% w/v to about 5% w/v, about 0.75% w/v to about 1% w/v, about 0.75% w/v to about 1.25% w/v, about 0.75% w/v to about 1.5% w/v, about 0.75% w/v to about 1.75% w/v, about 0.75% w/v to about 2% w/v, about 0.75% w/v to about 2.5% w/v, about 0.75% w/v to about 3% w/v, about 0.75% w/v to about 5% w/v, about 1% w/v to about 1.25% w/v, about 1% w/v to about 1.5% w/v, about 1% w/v to about 1.75% w/v, about 1% w/v to about 2% w/v, about 1% w/v to about 2.5% w/v, about 1% w/v to about 3% w/v, about 1% w/v to about 5% w/v, about 1.25% w/v to about 1.5% w/v, about 1.25% w/v to about 1.75% w/v, about 1.25% w/v to about 2% w/v, about 1.25% w/v to about 2.5% w/v, about 1.25% w/v to about 3% w/v, about 1.25% w/v to about 5% w/v, about 1.5% w/v to about 1.75% w/v, about 1.5% w/v to about 2% w/v, about 1.5% w/v to about 2.5% w/v, about 1.5% w/v to about 3% w/v, about 1.5% w/v to about 5% w/v, about 1.75% w/v to about 2% w/v, about 1.75% w/v to about 2.5% w/v, about 1.75% w/v to about 3% w/v, about 1.75% w/v to about 5% w/v, about 2% w/v to about 2.5% w/v, about 2% w/v to about 3% w/v, about 2% w/v to about 5% w/v, about 2.5% w/v to about 3% w/v, about 2.5% w/v to about 5% w/v, or about 3% w/v to about 5% w/v.

In some embodiments, the input composition to the bioreactor system 100 may comprise a nitrogen source 132. The nitrogen source may be ammonium sulfate, ammonium chloride, ammonium nitrate, sodium nitrate, yeast extract, yeast, or any combination thereof. In some cases, the nitrogen source may be added to the bioreactor system on a first day of a digestion process. In some cases, the nitrogen source may be added to the bioreactor system on a second day, third day, fourth day, or any day following a first day of a digestion process. The nitrogen source (e.g., ammonium sulfate) may be added to the reactor to maintain a concentration range of 0.02-0.2% w/v of nitrogen based on the total volume of the working fluid of the system. In some embodiments, the concentration of nitrogen that may be maintained can be based on the total concentration of all components of the working fluid in the at a point during the digestion process of the bioreactor system 100.

In some embodiments, the nitrogen source (e.g., ammonium sulfate) may be added to the reactor to maintain a concentration of nitrogen source in the working fluid of at least about 0.005% w/v, at least about 0.01% w/v, at least about 0.02% w/v, at least about 0.03% w/v, at least about 0.04% w/v, at least about 0.05% w/v, at least about 0.055% w/v, at least about 0.06% w/v, at least about 0.065% w/v, at least about 0.07% w/v, at least about 0.075% w/v, at least about 0.1% w/v, at least about 0.125% w/v, at least about 0.15% w/v, at least about 0.175% w/v, at least about 0.2% w/v, at least about 0.225% w/v, at least about 0.25% w/v, at least about 0.275% w/v, at least about 0.3% w/v, at least about 0.4% w/v, at least about 0.5% w/v, at least about 0.75% w/v, or at least about 1.0% w/v. In some embodiments, the nitrogen source (e.g., ammonium sulfate) may be added to the reactor to maintain a concentration of nitrogen source in the working fluid of at most about 1.0% w/v, at most about 0.75% w/v, at most about 0.5% w/v, at most about 0.4% w/v, at most about 0.3% w/v, at most about 0.275% w/v, at most about 0.25% w/v, at most about 0.225% w/v, at most about 0.2% w/v, at most about 0.175% w/v, at most about 0.15% w/v, at most about 0.125% w/v, at most about 0.1% w/v, at most about 0.075% w/v, at most about 0.07% w/v, at most about 0.065% w/v, at most about 0.06% w/v, at most about 0.055% w/v, at most about 0.05% w/v, at most about 0.04% w/v, at most about 0.03% w/v, at most about 0.02% w/v, at most about 0.01% w/v, or at most about 0.005% w/v.

In some embodiments, the nitrogen source (e.g., ammonium sulfate) may be added to the reactor to maintain a concentration of nitrogen source in the working fluid of about 0.03% w/v to about 1% w/v. In some embodiments, the nitrogen source (e.g., ammonium sulfate) may be added to the first reactor to maintain a concentration of nitrogen source in the working fluid of about 0.25% w/v to about 0.03% w/v, about 0.25% w/v to about 0.04% w/v, about 0.25% w/v to about 0.05% w/v, about 0.25% w/v to about 0.1% w/v, about 0.25% w/v to about 0.125% w/v, about 0.25% w/v to about 0.15% w/v, about 0.25% w/v to about 0.175% w/v, about 0.25% w/v to about 0.2% w/v, about 0.25% w/v to about 0.5% w/v, about 0.25% w/v to about 0.75% w/v, about 0.25% w/v to about 1% w/v, about 0.03% w/v to about 0.04% w/v, about 0.03% w/v to about 0.05% w/v, about 0.03% w/v to about 0.1% w/v, about 0.03% w/v to about 0.125% w/v, about 0.03% w/v to about 0.15% w/v, about 0.03% w/v to about 0.175% w/v, about 0.03% w/v to about 0.2% w/v, about 0.03% w/v to about 0.5% w/v, about 0.03% w/v to about 0.75% w/v, about 0.03% w/v to about 1% w/v, about 0.04% w/v to about 0.05% w/v, about 0.04% w/v to about 0.1% w/v, about 0.04% w/v to about 0.125% w/v, about 0.04% w/v to about 0.15% w/v, about 0.04% w/v to about 0.175% w/v, about 0.04% w/v to about 0.2% w/v, about 0.04% w/v to about 0.5% w/v, about 0.04% w/v to about 0.75% w/v, about 0.04% w/v to about 1% w/v, about 0.05% w/v to about 0.1% w/v, about 0.05% w/v to about 0.125% w/v, about 0.05% w/v to about 0.15% w/v, about 0.05% w/v to about 0.175% w/v, about 0.05% w/v to about 0.2% w/v, about 0.05% w/v to about 0.5% w/v, about 0.05% w/v to about 0.75% w/v, about 0.05% w/v to about 1% w/v, about 0.1% w/v to about 0.125% w/v, about 0.1% w/v to about 0.15% w/v, about 0.1% w/v to about 0.175% w/v, about 0.1% w/v to about 0.2% w/v, about 0.1% w/v to about 0.5% w/v, about 0.1% w/v to about 0.75% w/v, about 0.1% w/v to about 1% w/v, about 0.125% w/v to about 0.15% w/v, about 0.125% w/v to about 0.175% w/v, about 0.125% w/v to about 0.2% w/v, about 0.125% w/v to about 0.5% w/v, about 0.125% w/v to about 0.75% w/v, about 0.125% w/v to about 1% w/v, about 0.15% w/v to about 0.175% w/v, about 0.15% w/v to about 0.2% w/v, about 0.15% w/v to about 0.5% w/v, about 0.15% w/v to about 0.75% w/v, about 0.15% w/v to about 1% w/v, about 0.175% w/v to about 0.2% w/v, about 0.175% w/v to about 0.5% w/v, about 0.175% w/v to about 0.75% w/v, about 0.175% w/v to about 1% w/v, about 0.2% w/v to about 0.5% w/v, about 0.2% w/v to about 0.75% w/v, about 0.2% w/v to about 1% w/v, about 0.5% w/v to about 0.75% w/v, about 0.5% w/v to about 1% w/v, or about 0.75% w/v to about 1% w/v.

In some cases, the quantities added of the carbon and/or nitrogen sources to the system may be relative to the retention time of the system and can be adjusted accordingly if the flow rate changes. For example, a bioreactor system with a longer retention time may have a larger amount of a carbon source and/or nitrogen source added compared to an amount of a carbon source and/or nitrogen source added to a system with a short retention time.

In some embodiments, the input composition to the bioreactor system 100 may comprise micronutrients 133. Micronutrients may comprise important elements for microorganisms to support physiological functions. The micronutrients may comprise less than about 1%, less than about 2%, less than about 3%, less than about 4%, or less than about 5% of a dry weight of a plant. Elemental micronutrients may comprise boron, zinc, manganese, copper, chlorine, molybdenum, or any combination thereof. The addition of micronutrients or compounds that provide micronutrients may enrich a population of the microbial strain and/or microbes of a microbial consortium of a working fluid of a bioreactor system as described herein. Addition of inorganic nutrients may enrich a population of the microbial strain and/or microbes of a microbial consortium of a working fluid of a bioreactor system as described herein. The inorganic nutrients may comprise potassium chloride (KCl), dipotassium phosphate (K₂HPO₄), magnesium sulfate (MgSO₄), or any combination thereof.

Potassium chloride may be added into the reactor at a concentration range of at least about 0.005% w/v, at least about 0.01% w/v, at least about 0.015% w/v, at least about 0.02% w/v, at least about 0.025% w/v, at least about 0.03% w/v, at least about 0.035% w/v, at least about 0.04% w/v, at least about 0.045% w/v, at least about 0.05% w/v, at least about 0.055% w/v, at least about 0.06% w/v, at least about 0.07% w/v, at least about 0.08% w/v, at least about 0.09% w/v, or at least about 0.1% w/v. Potassium chloride may be added into the reactor at a concentration range of at most about 0.005% w/v, at most about 0.01% w/v, at most about 0.015% w/v, at most about 0.02% w/v, at most about 0.025% w/v, at most about 0.03% w/v, at most about 0.035% w/v, at most about 0.04% w/v, at most about 0.045% w/v, at most about 0.05% w/v, at most about 0.055% w/v, at most about 0.06% w/v, at most about 0.07% w/v, at most about 0.08% w/v, at most about 0.09% w/v, or at most about 0.1% w/v.

Dipotassium phosphate may be added into the reactor at a concentration range of at least about 0.005% w/v, at least about 0.01% w/v, at least about 0.015% w/v, at least about 0.02% w/v, at least about 0.025% w/v, at least about 0.03% w/v, at least about 0.035% w/v, at least about 0.04% w/v, at least about 0.045% w/v, at least about 0.05% w/v, at least about 0.055% w/v, at least about 0.06% w/v, at least about 0.07% w/v, at least about 0.08% w/v, at least about 0.09% w/v, or at least about 0.1% w/v. Dipotassium phosphate may be added into the reactor at a concentration range of at most about 0.005% w/v, at most about 0.01% w/v, at most about 0.015% w/v, at most about 0.02% w/v, at most about 0.025% w/v, at most about 0.03% w/v, at most about 0.035% w/v, at most about 0.04% w/v, at most about 0.045% w/v, at most about 0.05% w/v, at most about 0.055% w/v, at most about 0.06% w/v, at most about 0.07% w/v, at most about 0.08% w/v, at most about 0.09% w/v, or at most about 0.1% w/v.

Magnesium sulfate may be added into the reactor at a concentration range of at least about 0.005% w/v, at least about 0.01% w/v, at least about 0.015% w/v, at least about 0.02% w/v, at least about 0.025% w/v, at least about 0.03% w/v, at least about 0.035% w/v, at least about 0.04% w/v, at least about 0.045% w/v, at least about 0.05% w/v, at least about 0.055% w/v, at least about 0.06% w/v, at least about 0.07% w/v, at least about 0.08% w/v, at least about 0.09% w/v, or at least about 0.1% w/v. Magnesium sulfate may be added into the reactor at a concentration range of at most about 0.005% w/v, at most about 0.01% w/v, at most about 0.015% w/v, at most about 0.02% w/v, at most about 0.025% w/v, at most about 0.03% w/v, at most about 0.035% w/v, at most about 0.04% w/v, at most about 0.045% w/v, at most about 0.05% w/v, at most about 0.055% w/v, at most about 0.06% w/v, at most about 0.07% w/v, at most about 0.08% w/v, at most about 0.09% w/v, or at most about 0.1% w/v.

In some embodiments, micronutrients may be added on a first day, a second day, a third day, a fourth day, and/or a later day of a digestion process using bioreactor system 100 based on the total volume of the working fluid of the system.

In some embodiments, a concentration of range of 0.1-5.0% v/v of floc of a Phosphate Solubilizing Technology (PST/PwST), PST WB, may also be added as an input to the reactor on the span of the hydraulic retention time of the bioreactor system. A hydraulic retention time may comprise an amount of time a working fluid is maintained in a container (e.g., is not transferred out) of a bioreactor system as described herein. In some embodiments, the concentration range of PST WB input to the system may be at least about 0.05% v/v, at least about 0.1% v/v, at least about 0.2% v/v, at least about 0.3% v/v, at least about 0.4% v/v, at least about 0.5% v/v, at least about 0.6% v/v, at least about 0.7% v/v, at least about 0.8% v/v, at least about 0.9% v/v, at least about 1.0% v/v, at least about 1.5% v/v, at least about 2.0% v/v, at least about 2.5% v/v, at least about 3.0% v/v, at least about 3.5% v/v, at least about 4.0% v/v, at least about 4.5% v/v, at least about 5.0% v/v, at least about 5.5% v/v, at least about 6.0% v/v, at least about 6.5% v/v, at least about 7.0% v/v, at least about 7.5% v/v, at least about 10.0% v/v, at least about 12.5% v/v, at least about 15.0% v/v. In some embodiments, the concentration range of PST WB input to the system may be at most about 0.05% v/v, at most about 0.1% v/v, at most about 0.2% v/v, at most about 0.3% v/v, at most about 0.4% v/v, at most about 0.5% v/v, at most about 0.6% v/v, at most about 0.7% v/v, at most about 0.8% v/v, at most about 0.9% v/v, at most about 1.0% v/v, at most about 1.5% v/v, at most about 2.0% v/v, at most about 2.5% v/v, at most about 3.0% v/v, at most about 3.5% v/v, at most about 4.0% v/v, at most about 4.5% v/v, at most about 5.0% v/v, at most about 5.5% v/v, at most about 6.0% v/v, at most about 6.5% v/v, at most about 7.0% v/v, at most about 7.5% v/v, at most about 10.0% v/v, at most about 12.5% v/v, or at most about 15.0% v/v.

In some cases, the PST WB provides a phosphorous source of rock phosphate to the working fluid of the bioreactor system 100. The PST WB can provide a first consortium to the bioreactor system 100 in order to populate a working fluid of a first reactor. In some embodiments, a ratio of floc: SPN may be 1:1, 1:2, 1:3, 1:4, 1:5, 1:6, 1:7, 1:8, 1:9, 1:10, 10:1, 9:1, 8:1, 7:1, 6:1, 5:1, 4:1, 3:1, or 2:1. The floc: SPN may be a combined mixture of floc (e.g., biosolids) and supernatant from the clarifier. In some embodiments, the PST WB may be added into the first reactor 111. In some embodiments, the PST WB may be added into a second reactor 112, a third reactor 113, and/or a fourth reactor 114. In some embodiments, a concentration of range of 0.5-8% v/v of whole broth (WB) of a water-based Phosphate Solubilizing Technology (PwST), PwST WB (in a ratio of 1:1 floc: supernatant (SPN)), may also be added to a reactor on the span of the hydraulic retention time of the bioreactor system. In some embodiments, the PwST WB may be added into the first reactor 111. In some embodiments, the PwST WB may be added into a second reactor 112, a third reactor 113, and/or a fourth reactor 114. The PST WB and/or PwST WB may provide a phosphorous source to the bioreactor system. Addition of the PST WB and/or PwST WB may provide a first consortium to populate a working fluid of a first reactor 111 of the bioreactor system 100. A concentration of the PST WB or PwST WB may be based on a total concentration of components of a working fluid of a bioreactor system 100.

In some embodiments, the concentration range of PST WB or PwST WB input to the system may be at least about 0.05% v/v, at least about 0.1% v/v, at least about 0.2% v/v, at least about 0.3% v/v, at least about 0.4% v/v, at least about 0.5% v/v, at least about 0.6% v/v, at least about 0.7% v/v, at least about 0.8% v/v, at least about 0.9% v/v, at least about 1.0% v/v, at least about 1.5% v/v, at least about 2.0% v/v, at least about 2.5% v/v, at least about 3.0% v/v, at least about 3.5% v/v, at least about 4.0% v/v, at least about 4.5% v/v, at least about 5.0% v/v, at least about 5.5% v/v, at least about 6.0% v/v, at least about 6.5% v/v, at least about 7.0% v/v, at least about 7.5% v/v, at least about 10.0% v/v, at least about 12.5% v/v, at least about 15.0% v/v. In some embodiments, the concentration range of PST WB or PwST WB input to the system may be at most about 0.05% v/v, at most about 0.1% v/v, at most about 0.2% v/v, at most about 0.3% v/v, at most about 0.4% v/v, at most about 0.5% v/v, at most about 0.6% v/v, at most about 0.7% v/v, at most about 0.8% v/v, at most about 0.9% v/v, at most about 1.0% v/v, at most about 1.5% v/v, at most about 2.0% v/v, at most about 2.5% v/v, at most about 3.0% v/v, at most about 3.5% v/v, at most about 4.0% v/v, at most about 4.5% v/v, at most about 5.0% v/v, at most about 5.5% v/v, at most about 6.0% v/v, at most about 6.5% v/v, at most about 7.0% v/v, at most about 7.5% v/v, at most about 10.0% v/v, at most about 12.5% v/v, at most about 15.0% v/v.

In some embodiments, the input composition, as described herein, may comprise calcium carbonate. The calcium carbonate may be added to the first reactor to at a concentration range of 0.02-0.2% w/v based on the span of the hydraulic retention time of the bioreactor system. In some embodiments, the calcium carbonate may be added to the first reactor to at a concentration range of a calcium carbonate in a working fluid of at least about 0.005%, at least about 0.075%, at least about 0.01%, at least about 0.015%, at least about 0.02%, at least about 0.025%, at least about 0.03%, at least about 0.05%, at least about 0.075%, at least about 0.1%, at least about 0.125%, at least about 0.15%, at least about 0.175%, at least about 0.2%, at least about 0.225%, at least about 0.25%, at least about 0.275%, at least about 0.3%, at least about 0.4%, at least about 0.5%, at least about 0.6%, at least about 0.75%, at least about 1.0%, at least about 1.5%, at least about 2.0%, at least about 3.0%, at least about 4.0%, or at least about 5.0% w/v. In some embodiments, the calcium carbonate may be added to the first reactor to at a concentration range of a calcium carbonate in a working fluid of at most about 0.005%, at most about 0.075%, at most about 0.01%, at most about 0.015%, at most about 0.02%, at most about 0.025%, at most about 0.03%, at most about 0.05%, at most about 0.075%, at most about 0.1%, at most about 0.125%, at most about 0.15%, at most about 0.175%, at most about 0.2%, at most about 0.225%, at most about 0.25%, at most about 0.275%, at most about 0.3%, at most about 0.4%, at most about 0.5%, at most about 0.6%, at most about 0.75%, at most about 1.0%, at most about 1.5%, at most about 2.0%, at most about 3.0%, at most about 4.0%, or at most about 5.0% w/v.

In some embodiments, the input composition, as described herein, may comprise plant-based materials (e.g., soluble plant-based materials). The plant-based material may be soy flour, corn flour, cereal flour, corn gluten, soy flour protein, soy protein hydrolysate, lentil flour, chickpea flour, green pea flour, yellow pea flour, white bean flour, or any combination thereof. The plant-based material (e.g., soy flour) may be added to the first reactor to at a concentration range of 0.2-3% w/v based on the span of the hydraulic retention time of the bioreactor system. In some embodiments, the plant-based material (e.g., soy flour) may be added to the first reactor at a concentration range of plant-based material in the working fluid of at least about 0.05%, at least about 0.075%, at least about 0.1%, at least about 0.125%, at least about 0.15%, at least about 0.175%, at least about 0.2%, at least about 0.25%, at least about 0.3%, at least about 0.5%, at least about 0.75%, at least about 1.0%, at least about 1.5%, at least about 2.0%, at least about 2.5%, at least about 3.0%, at least about 3.5%, at least about 4.0%, at least about 5.0%, at least about 6.0%, at least about 7.0%, at least about 8.0%, at least about 9.0%, or at least about 10.0% w/v. In some embodiments, the plant-based material (e.g., soy flour) may be added to the first reactor at a concentration range of plant-based material in the working fluid of at most about 0.05%, at most about 0.075%, at most about 0.1%, at most about 0.125%, at most about 0.15%, at most about 0.175%, at most about 0.2%, at most about 0.25%, at most about 0.3%, at most about 0.5%, at most about 0.75%, at most about 1.0%, at most about 1.5%, at most about 2.0%, at most about 2.5%, at most about 3.0%, at most about 3.5%, at most about 4.0%, at most about 5.0%, at most about 6.0%, at most about 7.0%, at most about 8.0%, at most about 9.0%, or at most about 10.0% w/v.

In some cases, a bioreactor system 100 may be inoculated with an isolate or combination of isolates. In some cases, a bioreactor system 100 may not be reinoculated with an isolate or combination of isolates following a first inoculation. In some cases, a bioreactor system 100 may be reinoculated with an isolate or combination of isolates at least every 20 days, at least every 50 days, at least every 100 days, at least every 200 days, at least every 300 days, at least every 400 days, at least every 500 days, or more. In some cases, a bioreactor system 100 may be reinoculated with an isolate or combination of isolates at most every 500 days, at most every 400 days, at most every 300 days, at most every 200 days, at most every 100 days, at most every 50 days, at most every 20 days, or less. In some cases, a bioreactor system 100 may be inoculated with an isolate or combination of isolates on day 1 of a digestion process and reinoculated 1, 2, 3, 4, or 5 more times after day 1 of the digestion process.

The inoculum of a microbe may be an isolated microbe. An isolated microbe can comprise a microbe grown or enriched outside of a natural environment (e.g., in a culture medium or a streak plate method). In some embodiments, the inoculum of a microbe may comprise a mixture of two or more isolated microbes. The inoculum of a microbe may comprise a mixture of at least about 2, at least about 3, at least about 4, at least about 5, at least about 6, at least about 7, at least about 8, or more isolated microbes.

In some embodiments, the bioreactor system 100 may be inoculated with isolates MS4666, MS4689, MS3900, MS3907, MS4921, MS4687, MS2839, MS1835, or any combination thereof. A bioreactor system 100 may be inoculated with zinc solubilizing spore former. A bioreactor system 100 may be inoculated with phosphate solubilizing spore former. A bioreactor system 100 may be inoculated with nitrogen fixer. A bioreactor system 100 may be inoculated with zinc solubilizing rhizobacteria. A bioreactor system 100 may be inoculated with phosphate solubilizing rhizobacteria. A bioreactor system 100 may be inoculated with nitrogen fixing rhizobacteria. In some embodiments, the population of the microbe does not decrease by more than 60%, by more than 50%, by more than 45%, by more than 40%, by more than 35%, by more than 30%, by more than 25%, by more than 20%, by more than 15%, by more than 10%, or by more than 5% between a first and a second transferring of the isolate (e.g., microbe). In some embodiments, the population of the microbe is maintained by at least about 5%, at least 10%, at least about 15%, at least about 20%, at least about 25%, at least about 30%, at least about 35%, at least about 40%, at least about 45%, at least about 50%, at least about 55%, at least about 60%, or at least about 75%.

In some embodiments, the incubation time, i.e., the amount of time that the working fluid is retained in the bioreactor system 100 before the product is removed, of the bioreactor system 100 may be 7 days. In some embodiments, the incubation time of the bioreactor system may be at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 25 days, at least about 30 days, or at least about 50 days. In some embodiments, the incubation time of the bioreactor system 100 may be at most about 50 days, at most about 30 days, at most about 25 days, at most about 20 days, at most about 19 days, at most about 18 days, at most about 17 days, at most about 16 days, at most about 15 days, at most about 14 days, at most about 13 days, at most about 12 days, at most about 11 days, at most about 10 days, at most about 9 days, at most about 8 days, at most about 7 days, at most about 6 days, at most about 5 days, at most about 4 days, at most about 3 days, at most about 2 days, or at most about 1 day.

In some embodiments, the incubation time of the bioreactor system may be about 1 day to about 30 days. In some embodiments, the incubation time of the bioreactor system may be about 1 day to about 3 days, about 1 day to about 5 days, about 1 day to about 8 days, about 1 day to about 10 days, about 1 day to about 12 days, about 1 day to about 14 days, about 1 day to about 16 days, about 1 day to about 18 days, about 1 day to about 20 days, about 1 day to about 25 days, about 1 day to about 30 days, about 3 days to about 5 days, about 3 days to about 8 days, about 3 days to about 10 days, about 3 days to about 12 days, about 3 days to about 14 days, about 3 days to about 16 days, about 3 days to about 18 days, about 3 days to about 20 days, about 3 days to about 25 days, about 3 days to about 30 days, about 5 days to about 8 days, about 5 days to about 10 days, about 5 days to about 12 days, about 5 days to about 14 days, about 5 days to about 16 days, about 5 days to about 18 days, about 5 days to about 20 days, about 5 days to about 25 days, about 5 days to about 30 days, about 8 days to about 10 days, about 8 days to about 12 days, about 8 days to about 14 days, about 8 days to about 16 days, about 8 days to about 18 days, about 8 days to about 20 days, about 8 days to about 25 days, about 8 days to about 30 days, about 10 days to about 12 days, about 10 days to about 14 days, about 10 days to about 16 days, about 10 days to about 18 days, about 10 days to about 20 days, about 10 days to about 25 days, about 10 days to about 30 days, about 12 days to about 14 days, about 12 days to about 16 days, about 12 days to about 18 days, about 12 days to about 20 days, about 12 days to about 25 days, about 12 days to about 30 days, about 14 days to about 16 days, about 14 days to about 18 days, about 14 days to about 20 days, about 14 days to about 25 days, about 14 days to about 30 days, about 16 days to about 18 days, about 16 days to about 20 days, about 16 days to about 25 days, about 16 days to about 30 days, about 18 days to about 20 days, about 18 days to about 25 days, about 18 days to about 30 days, about 20 days to about 25 days, about 20 days to about 30 days, or about 25 days to about 30 days.

In some embodiments, a bioreactor system may comprise two or more bioreactor systems 100 with each reactor producing a base product. Each reactor may operate individually and produce a base product.

In some embodiments, water may act as a hydraulic source 140 and, when coupled to the first reactor 111, provides continuous flow of water to the first reactor 111 at an electrical conductivity of at least about 100 microsiemens/centimeter (μS/cm), In some embodiments, the water tank 141 when coupled to the first reactor 111 provides continuous flow of water to the first reactor 111 at an electrical conductivity of at least about 100 microsiemens/centimeter (μS/cm), at least about 150 μS/cm, at least about 200 μS/cm, at least about 250 μS/cm, at least about 300 μS/cm, at least about 350 μS/cm, at least about 400 μS/cm, at least about 450 μS/cm, at least about 500 μS/cm, at least about 550 μS/cm, or at least about 600 μS/cm.

In some embodiments, the water tank 141 when coupled to the first reactor 111 provides continuous flow of water to the first reactor 111 at an electrical conductivity of at most about 600 μS/cm, at most about 550 μS/cm, at most about 500 μS/cm, at most about 450 μS/cm, at most about 400 μS/cm, at most about 350 μS/cm, at most about 300 μS/cm, at most about 250 μS/cm, at most about 200 μS/cm, at most about 150 μS/cm, or at most about 100 μS/cm.

In some embodiments, the water tank 141 when coupled to the first reactor 111 provides continuous flow of water to the first reactor 111 at an electrical conductivity of about 50 μS/cm to about 800 μS/cm. In some embodiments, the water tank 141 when coupled to the first reactor 111 provides continuous flow of water to the first reactor 111 at an electrical conductivity of about 50 μS/cm to about 100 μS/cm, about 50 μS/cm to about 200 μS/cm, about 50 μS/cm to about 250 μS/cm, about 50 μS/cm to about 300 μS/cm, about 50 μS/cm to about 350 μS/cm, about 50 μS/cm to about 400 μS/cm, about 50 μS/cm to about 450 μS/cm, about 50 μS/cm to about 500 μS/cm, about 50 μS/cm to about 600 μS/cm, about 50 μS/cm to about 700 μS/cm, about 50 μS/cm to about 800 μS/cm, about 100 μS/cm to about 200 μS/cm, about 100 μS/cm to about 250 μS/cm, about 100 μS/cm to about 300 μS/cm, about 100 μS/cm to about 350 μS/cm, about 100 μS/cm to about 400 μS/cm, about 100 μS/cm to about 450 μS/cm, about 100 μS/cm to about 500 μS/cm, about 100 μS/cm to about 600 μS/cm, about 100 μS/cm to about 700 μS/cm, about 100 μS/cm to about 800 μS/cm, about 200 μS/cm to about 250 μS/cm, about 200 μS/cm to about 300 μS/cm, about 200 μS/cm to about 350 μS/cm, about 200 μS/cm to about 400 μS/cm, about 200 μS/cm to about 450 μS/cm, about 200 μS/cm to about 500 μS/cm, about 200 μS/cm to about 600 μS/cm, about 200 μS/cm to about 700 μS/cm, about 200 μS/cm to about 800 μS/cm, about 250 μS/cm to about 300 μS/cm, about 250 μS/cm to about 350 μS/cm, about 250 μS/cm to about 400 μS/cm, about 250 μS/cm to about 450 μS/cm, about 250 μS/cm to about 500 μS/cm, about 250 μS/cm to about 600 μS/cm, about 250 μS/cm to about 700 μS/cm, about 250 μS/cm to about 800 μS/cm, about 300 μS/cm to about 350 μS/cm, about 300 μS/cm to about 400 μS/cm, about 300 μS/cm to about 450 μS/cm, about 300 μS/cm to about 500 μS/cm, about 300 μS/cm to about 600 μS/cm, about 300 μS/cm to about 700 μS/cm, about 300 μS/cm to about 800 μS/cm, about 350 μS/cm to about 400 μS/cm, about 350 μS/cm to about 450 μS/cm, about 350 μS/cm to about 500 μS/cm, about 350 μS/cm to about 600 μS/cm, about 350 μS/cm to about 700 μS/cm, about 350 μS/cm to about 800 μS/cm, about 400 μS/cm to about 450 μS/cm, about 400 μS/cm to about 500 μS/cm, about 400 μS/cm to about 600 μS/cm, about 400 μS/cm to about 700 μS/cm, about 400 μS/cm to about 800 μS/cm, about 450 μS/cm to about 500 μS/cm, about 450 μS/cm to about 600 μS/cm, about 450 μS/cm to about 700 μS/cm, about 450 μS/cm to about 800 μS/cm, about 500 μS/cm to about 600 μS/cm, about 500 μS/cm to about 700 μS/cm, about 500 μS/cm to about 800 μS/cm, about 600 μS/cm to about 700 μS/cm, about 600 μS/cm to about 800 μS/cm, or about 700 μS/cm to about 800 μS/cm.

In some embodiments, the hydraulic rate of flow through the bioreactor system may be 4.6 ml/min to maintain a retention time of 10 days and can be varied accordingly for a retention time from 7 to 21 days depending on production volume requirements. In some embodiments, the hydraulic rate of flow through the bioreactor system may be a rate of 15 ml/min or 11 ml/min. In some embodiments, the hydraulic rate of flow through the digestion system may be a rate of at least about 1 ml/min, 2 ml/min, 3 ml/min, 4 ml/min, 4.5 ml/min, 5 ml/min, 5.5 ml/min, 6 ml/min, 6.5 ml/min, 7 ml/min, 7.5 ml/min, 8 ml/min, 9 ml/min, 10 ml/min, 11 ml/min, 12 ml/min, 13 ml/min, 14 ml/min, 14.5 ml/min, 15 ml/min, 15.5 ml/min, 16 ml/min, 16.5 ml/min, 17 ml/min, 17.5 ml/min, 20 ml/min, 22.5 ml/min, 25 ml/min, or greater than about 25 ml/min. In some embodiments, the hydraulic rate of flow through the digestion system may be a rate of at most about 25 ml/min 22.5 ml/min, 20 ml/min, 17.5 ml/min, 17 ml/min, 16.5 ml/min, 16 ml/min, 15.5 ml/min, 15 ml/min, 14.5 ml/min, 14 ml/min, 13 ml/min, 12 ml/min, 11 ml/min, 10 ml/min, 9 ml/min, 8 ml/min, 7.5 ml/min, 7 ml/min, 6.5 ml/min, 6 ml/min, 5.5 ml/min, 5 ml/min, 4.5 ml/min, 4 ml/min, 3 ml/min, 2 ml/min, 1 ml/min, or less than about 1 ml/min.

An amount of biosolids may be maintained in a bioreactor system. Maintenance of biosolids may support enrichment and/or growth of a population of the microbial strain, microbes of a microbial consortium, nutrients, or additional components of a bioreactor system as described herein. In some embodiments, as biosolids accumulate over time in the clarifier container 330, a range of at least about 20-25% biosolids v/v may be maintained in the bioreactor system. In some embodiments, additional floc may be harvested from the bioreactor system and removed. In some embodiments, at least about 5% biosolids v/v, at least about 10% biosolids v/v, at least about 15% biosolids v/v, at least about 16% biosolids v/v, at least about 17% biosolids v/v, at least about 18% biosolids v/v, at least about 19% biosolids v/v, at least about 20% biosolids v/v, at least about 21% biosolids v/v, at least about 22% biosolids v/v, at least about 23% biosolids v/v, at least about 24% biosolids v/v, at least about 25% biosolids v/v, at least about 26% biosolids v/v, at least about 27% biosolids v/v, at least about 28% biosolids v/v, at least about 29% biosolids v/v, at least about 30% biosolids v/v, at least about 35% biosolids v/v, at least about 40% biosolids v/v, at least about 45% biosolids v/v, or at least about 50% biosolids v/v may be maintained in the bioreactor system.

In some embodiments, at most about 50% biosolids v/v, at most about 45% biosolids v/v, at most about 40% biosolids v/v, at most about 35% biosolids v/v, at most about 30% biosolids v/v, at most about 29% biosolids v/v, at most about 28% biosolids v/v, at most about 27% biosolids v/v, at most about 26% biosolids v/v, at most about 25% biosolids v/v, at most about 24% biosolids v/v, at most about 23% biosolids v/v, at most about 22% biosolids v/v, at most about 21% biosolids v/v, at most about 20% biosolids v/v, at most about 19% biosolids v/v, at most about 18% biosolids v/v, at most about 17% biosolids v/v, at most about 16% biosolids v/v, at most about 15% biosolids v/v, at most about 14% biosolids v/v, at most about 13% biosolids v/v, at most about 12% biosolids v/v, at most about 11% biosolids v/v, at most about 10% biosolids v/v, or at most about 5% biosolids v/v may be maintained in the bioreactor system.

In some embodiments, about 0.1% biosolids v/v to about 60% biosolids v/v may be maintained in the bioreactor system. In some embodiments, about 0.1% biosolids v/v to about 1% biosolids v/v, about 0.1% biosolids v/v to about 5% biosolids v/v, about 0.1% biosolids v/v to about 10% biosolids v/v, about 0.1% biosolids v/v to about 15% biosolids v/v, about 0.1% biosolids v/v to about 20% biosolids v/v, about 0.1% biosolids v/v to about 25% biosolids v/v, about 0.1% biosolids v/v to about 30% biosolids v/v, about 0.1% biosolids v/v to about 35% biosolids v/v, about 0.1% biosolids v/v to about 40% biosolids v/v, about 0.1% biosolids v/v to about 50% biosolids v/v, about 0.1% biosolids v/v to about 60% biosolids v/v, about 1% biosolids v/v to about 5% biosolids v/v, about 1% biosolids v/v to about 10% biosolids v/v, about 1% biosolids v/v to about 15% biosolids v/v, about 1% biosolids v/v to about 20% biosolids v/v, about 1% biosolids v/v to about 25% biosolids v/v, about 1% biosolids v/v to about 30% biosolids v/v, about 1% biosolids v/v to about 35% biosolids v/v, about 1% biosolids v/v to about 40% biosolids v/v, about 1% biosolids v/v to about 50% biosolids v/v, about 1% biosolids v/v to about 60% biosolids v/v, about 5% biosolids v/v to about 10% biosolids v/v, about 5% biosolids v/v to about 15% biosolids v/v, about 5% biosolids v/v to about 20% biosolids v/v, about 5% biosolids v/v to about 25% biosolids v/v, about 5% biosolids v/v to about 30% biosolids v/v, about 5% biosolids v/v to about 35% biosolids v/v, about 5% biosolids v/v to about 40% biosolids v/v, about 5% biosolids v/v to about 50% biosolids v/v, about 5% biosolids v/v to about 60% biosolids v/v, about 10% biosolids v/v to about 15% biosolids v/v, about 10% biosolids v/v to about 20% biosolids v/v, about 10% biosolids v/v to about 25% biosolids v/v, about 10% biosolids v/v to about 30% biosolids v/v, about 10% biosolids v/v to about 35% biosolids v/v, about 10% biosolids v/v to about 40% biosolids v/v, about 10% biosolids v/v to about 50% biosolids v/v, about 10% biosolids v/v to about 60% biosolids v/v, about 15% biosolids v/v to about 20% biosolids v/v, about 15% biosolids v/v to about 25% biosolids v/v, about 15% biosolids v/v to about 30% biosolids v/v, about 15% biosolids v/v to about 35% biosolids v/v, about 15% biosolids v/v to about 40% biosolids v/v, about 15% biosolids v/v to about 50% biosolids v/v, about 15% biosolids v/v to about 60% biosolids v/v, about 20% biosolids v/v to about 25% biosolids v/v, about 20% biosolids v/v to about 30% biosolids v/v, about 20% biosolids v/v to about 35% biosolids v/v, about 20% biosolids v/v to about 40% biosolids v/v, about 20% biosolids v/v to about 50% biosolids v/v, about 20% biosolids v/v to about 60% biosolids v/v, about 25% biosolids v/v to about 30% biosolids v/v, about 25% biosolids v/v to about 35% biosolids v/v, about 25% biosolids v/v to about 40% biosolids v/v, about 25% biosolids v/v to about 50% biosolids v/v, about 25% biosolids v/v to about 60% biosolids v/v, about 30% biosolids v/v to about 35% biosolids v/v, about 30% biosolids v/v to about 40% biosolids v/v, about 30% biosolids v/v to about 50% biosolids v/v, about 30% biosolids v/v to about 60% biosolids v/v, about 35% biosolids v/v to about 40% biosolids v/v, about 35% biosolids v/v to about 50% biosolids v/v, about 35% biosolids v/v to about 60% biosolids v/v, about 40% biosolids v/v to about 50% biosolids v/v, about 40% biosolids v/v to about 60% biosolids v/v, or about 50% biosolids v/v to about 60% biosolids v/v may be maintained in the bioreactor system.

A retention time may comprise a time a population of an inoculated microbial strain spends in a bioreactor system or a time a population of the microbial strain spends following transfer into a first container and until collection from the bioreactor system. A longer retention time may be advantageous for growth or enrichment of a population of the microbial strain of the bioreactor system. A shorter retention time may be advantageous for growth or enrichment of a population of the microbial strain of the bioreactor system. In some embodiments, the retention time of the bioreactor system may be 10 days.

In some embodiments, the retention time of the bioreactor system may be at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 22 days, or at least about 25 days.

In some embodiments, the retention time of the bioreactor system may be at most about 25 days, at most about 22 days, at most about 20 days, at most about 19 days, at most about 18 days, at most about 17 days, at most about 16 days, at most about 15 days, at most about 14 days, at most about 13 days, at most about 12 days, at most about 11 days, at most about 10 days, at most about 9 days, at most about 8 days, at most about 7 days, at most about 6 days, at most about 5 days, at most about 4 days, at most about 3 days, at most about 2 days, or at most about 1 day.

In some embodiments, the retention time of the bioreactor system may be about 1 day to about 30 days. In some embodiments, the retention time of the bioreactor system may be about 1 day to about 3 days, about 1 day to about 5 days, about 1 day to about 8 days, about 1 day to about 10 days, about 1 day to about 12 days, about 1 day to about 14 days, about 1 day to about 16 days, about 1 day to about 18 days, about 1 day to about 20 days, about 1 day to about 25 days, about 1 day to about 30 days, about 3 days to about 5 days, about 3 days to about 8 days, about 3 days to about 10 days, about 3 days to about 12 days, about 3 days to about 14 days, about 3 days to about 16 days, about 3 days to about 18 days, about 3 days to about 20 days, about 3 days to about 25 days, about 3 days to about 30 days, about 5 days to about 8 days, about 5 days to about 10 days, about 5 days to about 12 days, about 5 days to about 14 days, about 5 days to about 16 days, about 5 days to about 18 days, about 5 days to about 20 days, about 5 days to about 25 days, about 5 days to about 30 days, about 8 days to about 10 days, about 8 days to about 12 days, about 8 days to about 14 days, about 8 days to about 16 days, about 8 days to about 18 days, about 8 days to about 20 days, about 8 days to about 25 days, about 8 days to about 30 days, about 10 days to about 12 days, about 10 days to about 14 days, about 10 days to about 16 days, about 10 days to about 18 days, about 10 days to about 20 days, about 10 days to about 25 days, about 10 days to about 30 days, about 12 days to about 14 days, about 12 days to about 16 days, about 12 days to about 18 days, about 12 days to about 20 days, about 12 days to about 25 days, about 12 days to about 30 days, about 14 days to about 16 days, about 14 days to about 18 days, about 14 days to about 20 days, about 14 days to about 25 days, about 14 days to about 30 days, about 16 days to about 18 days, about 16 days to about 20 days, about 16 days to about 25 days, about 16 days to about 30 days, about 18 days to about 20 days, about 18 days to about 25 days, about 18 days to about 30 days, about 20 days to about 25 days, about 20 days to about 30 days, or about 25 days to about 30 days.

In some embodiment, the bioreactor system may produce 1.75 gallons of base product flowing from the clarifiers per day. In some embodiment, the bioreactor system may produce 4.29 gallons of base product flowing from the clarifiers per day. In some embodiment, the bioreactor system may produce 6 gallons of base product flowing from the clarifiers per day. In some embodiments, the bioreactor system may produce at least about 0.5, at least about 0.75, at least about 1.0, at least about 1.25, at least about 1.5, at least about 1.75, at least about 2.0, at least about 2.25, at least about 2.5, at least about 2.75, at least about 3.0, at least about 3.5, at least about 4.0, at least about 4.5, at least about 5.0, at least about 6.0, at least about 7.0, at least about 8.0, at least about 9.0, at least about 10.0, at least about 12.0, or at least about 15.0 gallons of base product flowing from the clarifiers per day. In some embodiments, the bioreactor system may produce at most about 0.5, at most about 0.75, at most about 1.0, at most about 1.25, at most about 1.5, at most about 1.75, at most about 2.0, at most about 2.25, at most about 2.5, at most about 2.75, at most about 3.0, at most about 3.5, at most about 4.0, at most about 4.5, at most about 5.0, at most about 6.0, at most about 7.0, at most about 8.0, at most about 9.0, at most about 10.0, at most about 12.0, at most about 15.0 gallons of base product flowing from the clarifiers per day.

In some embodiment, the bioreactor system may produce 12.25 gallons of base product flowing from the clarifiers per week. In some embodiment, the bioreactor system may produce 42 gallons of base product flowing from the clarifiers per week. In some embodiment, the bioreactor system may produce 30 gallons of base product flowing from the clarifiers per week. In some embodiment, the bioreactor system may produce at least about 2 gallons, at least about 5 gallons, at least about 7.5 gallons, at least about 10 gallons, at least about 12.5 gallons, at least about 15 gallons, at least about 20 gallons, at least about 25 gallons, at least about 30 gallons, at least about 35 gallons, at least about 40 gallons, at least about 45 gallons, at least about 50 gallons, at least about 55 gallons, at least about 60 gallons, at least about 70 gallons, at least about 80 gallons, at least about 90 gallons, or at least about 100 gallons of base product flowing from the clarifiers per week. In some embodiment, the bioreactor system may produce at most about 2 gallons, at most about 5 gallons, at most about 7.5 gallons, at most about 10 gallons, at most about 12.5 gallons, at most about 15 gallons, at most about 20 gallons, at most about 25 gallons, at most about 30 gallons, at most about 35 gallons, at most about 40 gallons, at most about 45 gallons, at most about 50 gallons, at most about 55 gallons, at most about 60 gallons, at most about 70 gallons, at most about 80 gallons, at most about 90 gallons, or at most about 100 gallons of base product flowing from the clarifiers per week.

In some embodiments, the bioreactor system can have an output of base product of at least about 100 gallons, at least about 200 gallons, at least about 300 gallons, at least about 400 gallons, at least about 500 gallons, at least about 750 gallons, at least about 1,000 gallons, at least about 1,500 gallons, at least about 2,000 gallons, at least about 2,500 gallons, at least about 5,000 gallons, at least about 7,500 gallons, at least about 10,000 gallons, at least about 15,000 gallons, at least about 20,000 gallons, at least about 25,000 gallons, at least about 50,000 gallons, at least about 60,000 gallons, at least about 70,000 gallons, at least about 80,000 gallons, at least about 90,000 gallons, or at least about 100,000 gallons per week. In some embodiments, the bioreactor system can have an output of base product of at most about 100 gallons, at most about 200 gallons, at most about 300 gallons, at most about 400 gallons, at most about 500 gallons, at most about 750 gallons, at most about 1,000 gallons, at most about 1,500 gallons, at most about 2,000 gallons, at most about 2,500 gallons, at most about 5,000 gallons, at most about 7,500 gallons, at most about 10,000 gallons, at most about 15,000 gallons, at most about 20,000 gallons, at most about 25,000 gallons, at most about 50,000 gallons, at most about 60,000 gallons, at most about 70,000 gallons, at most about 80,000 gallons, at most about 90,000 gallons, or at most about 100,000 gallons per week.

In some embodiments, the produced base products from the bioreactor system can have a pH of 7.39-8.88. In some embodiments, the produced base products from the bioreactor system can have a pH of at least about 4, at least about 4.5, at least about 5, at least about 5.5, at least about 6, at least about 6.5, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.1, at least about 8.2, at least about 8.3, at least about 8.4, at least about 8.5, at least about 8.6, at least about 8.7, at least about 8.8, at least about 8.9, at least about 9, at least about 9.1, at least about 9.2, at least about 9.3, at least about 9.4, at least about 9.5, at least about 9.6, at least about 9.7, at least about 9.8, at least about 9.9, or at least about 10. In some embodiments, the produced base products from the bioreactor system can have a pH of at most about 4, at most about 4.5, at most about 5, at most about 5.5, at most about 6, at most about 6.5, at most about 7, at most about 7.1, at most about 7.2, at most about 7.3, at most about 7.4, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 9.1, at most about 9.2, at most about 9.3, at most about 9.4, at most about 9.5, at most about 9.6, at most about 9.7, at most about 9.8, at most about 9.9, or at most about 10.

In some embodiments, the produced base products from the bioreactor system can have an electrical conductivity (Cond) range of 0.55-4.37 mS/cm. In some embodiments, the produced base products from the bioreactor system can have an electrical conductivity (Cond) range of at least about 0.1, at least about 0.2, at least about 0.3, at least about 0.4, at least about 0.5, at least about 0.6, at least about 0.7, at least about 0.8, at least about 0.9, at least about 1.0, at least about 1.5, at least about 2.0, at least about 2.5, at least about 3.0, at least about 3.5, at least about 4.0, at least about 4.5, at least about 5.0, at least about 5.5, at least about 6.0, at least about 6.5, at least about 7.0, at least about 7.5, at least about 8.0, at least about 8.5, at least about 9.0, at least about 9.5, or at least about 10.0 mS/cm. In some embodiments, the produced base products from the bioreactor system can have an electrical conductivity (Cond) range of at most about 0.1, at most about 0.2, at most about 0.3, at most about 0.4, at most about 0.5, at most about 0.6, at most about 0.7, at most about 0.8, at most about 0.9, at most about 1.0, at most about 1.5, at most about 2.0, at most about 2.5, at most about 3.0, at most about 3.5, at most about 4.0, at most about 4.5, at most about 5.0, at most about 5.5, at most about 6.0, at most about 6.5, at most about 7.0, at most about 7.5, at most about 8.0, at most about 8.5, at most about 9.0, at most about 9.5, or at most about 10.0 mS/cm.

In some embodiments, the produced base products from the bioreactor system can have a COD of 22-3600 mg/L. In some embodiments, the produced base products from the bioreactor system can have a COD of at least about 5, at least about 10, at least about 15, at least about 20, at least about 25, at least about 50, at least about 75, at least about 100, at least about 150, at least about 200, at least about 250, at least about 500, at least about 750, at least about 1,000, at least about 1,250, at least about 1,500, at least about 1,750, at least about 2,000, at least about 2,250, at least about 2,500, at least about 2,750, at least about 3,000, at least about 3,250, at least about 3,500, at least about 3,750, at least about 4,000, at least about 4,250, at least about 4,500, at least about 4,750, at least about 5,000, at least about 5,500, at least about 6,000, at least about 6,500, at least about 7,000, at least about 7,500, or at least about 10,000 mg/L. In some embodiments, the produced base products from the bioreactor system can have a COD of at most about 5, at most about 10, at most about 15, at most about 20, at most about 25, at most about 50, at most about 75, at most about 100, at most about 150, at most about 200, at most about 250, at most about 500, at most about 750, at most about 1,000, at most about 1,250, at most about 1,500, at most about 1,750, at most about 2,000, at most about 2,250, at most about 2,500, at most about 2,750, at most about 3,000, at most about 3,250, at most about 3,500, at most about 3,750, at most about 4,000, at most about 4,250, at most about 4,500, at most about 4,750, at most about 5,000, at most about 5,500, at most about 6,000, at most about 6,500, at most about 7,000, at most about 7,500, or at most about 10,000 mg/L.

In some embodiments, the produced base products from the bioreactor system can have a total nitrogen (N) content of 0.02-0.07%. In some embodiments, the produced base products from the bioreactor system can have a total nitrogen (N) content in a working fluid of the bioreactor system of at least about 0.005%, at least about 0.01%, at least about 0.0125%, at least about 0.015%, at least about 0.0175%, at least about 0.02%, at least about 0.0225%, at least about 0.025%, at least about 0.0275%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.2%, at least about 0.3%, at least about 0.4%, at least about 0.5%, at least about 0.75%, at least about 1.0%, at least about 1.5%, or at least about 2.0%. In some embodiments, the produced base products from the bioreactor system can have a total nitrogen (N) content in a working fluid of the bioreactor system of at most about 0.005%, at most about 0.01%, at most about 0.0125%, at most about 0.015%, at most about 0.0175%, at most about 0.02%, at most about 0.0225%, at most about 0.025%, at most about 0.0275%, at most about 0.03%, at most about 0.035%, at most about 0.04%, at most about 0.045%, at most about 0.05%, at most about 0.055%, at most about 0.06%, at most about 0.065%, at most about 0.07%, at most about 0.075%, at most about 0.08%, at most about 0.085%, at most about 0.09%, at most about 0.095%, at most about 0.1%, at most about 0.2%, at most about 0.3%, at most about 0.4%, at most about 0.5%, at most about 0.75%, at most about 1.0%, at most about 1.5%, or at most about 2.0%.

In some embodiments, the produced base products from the bioreactor system can have a total phosphorous (P) content of 0.00001%. In some embodiments, the produced base products from the bioreactor system can have a total phosphorous (P) content in a working fluid of the bioreactor system of at least about 0.00001%, at least about 0.00005%, at least about 0.0001%, at least about 0.0005%, at least about 0.001%, at least about 0.0025%, at least about 0.005%, at least about 0.0075%, at least about 0.01%, at least about 0.025%, at least about 0.05%, at least about 0.075%, at least about 0.1%, at least about 0.15%, or at least about 0.2%. In some embodiments, the produced base products from the bioreactor system can have a total phosphorous (P) content in a working fluid of the bioreactor system of at most about 0.00001%, at most about 0.00005%, at most about 0.0001%, at most about 0.0005%, at most about 0.001%, at most about 0.0025%, at most about 0.005%, at most about 0.0075%, at most about 0.01%, at most about 0.025%, at most about 0.05%, at most about 0.075%, at most about 0.1%, at most about 0.15%, or at most about 0.2%.

In some embodiments, the produced base products from the bioreactor system can have a total potassium (K) content of 0.082-0.094%. In some embodiments, the produced base products from the bioreactor system can have a total potassium (K) content in a working fluid of the bioreactor system of at least about 0.00001%, at least about 0.00005%, at least about 0.0001%, at least about 0.00025%, at least about 0.0005%, at least about 0.00075%, at least about 0.001%, at least about 0.0025%, at least about 0.005%, at least about 0.0075%, at least about 0.01%, at least about 0.025%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.11%, at least about 0.12%, at least about 0.13%, at least about 0.14%, at least about 0.15%, at least about 0.16%, at least about 0.17%, at least about 0.18%, at least about 0.19%, at least about 0.2%, at least about 0.25%, at least about 0.3%, at least about 0.35%, at least about 0.4%, at least about 0.45%, or at least about 0.5%. In some embodiments, the produced base products from the bioreactor system can have a total potassium (K) content in a working fluid of the bioreactor system of at most about 0.00001%, at most about 0.00005%, at most about 0.0001%, at most about 0.00025%, at most about 0.0005%, at most about 0.00075%, at most about 0.001%, at most about 0.0025%, at most about 0.005%, at most about 0.0075%, at most about 0.01%, at most about 0.025%, at most about 0.05%, at most about 0.055%, at most about 0.06%, at most about 0.065%, at most about 0.07%, at most about 0.075%, at most about 0.08%, at most about 0.085%, at most about 0.09%, at most about 0.095%, at most about 0.1%, at most about 0.11%, at most about 0.12%, at most about 0.13%, at most about 0.14%, at most about 0.15%, at most about 0.16%, at most about 0.17%, at most about 0.18%, at most about 0.19%, at most about 0.2%, at most about 0.25%, at most about 0.3%, at most about 0.35%, at most about 0.4%, at most about 0.45%, or at most about 0.5%.

In some embodiments, the working fluid in each reactor described herein may be circulated within the reactor in such a way as to minimize surface disruption, which maintains the dissolved oxygen within the reactors between 0.14 and 0.5 mg/L. In some embodiments, the dissolved oxygen within the reactors may be at least about 0.05, at least about 0.075, at least about 0.1, at least about 0.125, at least about 0.15, at least about 0.175, at least about 0.2, at least about 0.25, at least about 0.3, at least about 0.35, at least about 0.4, at least about 0.45, at least about 0.5, at least about 0.55, at least about 0.6, at least about 0.65, at least about 0.7, at least about 0.75, at least about 0.8, at least about 0.9, at least about 1.0, at least about 1.25, at least about 1.5, at least about 1.75, at least about 2.0, at least about 2.5, at least about 5.0, at least about 7.5, or at least about 10.0 mg/L. In some embodiments, the dissolved oxygen within the reactors may be at most about 0.05, at most about 0.075, at most about 0.1, at most about 0.125, at most about 0.15, at most about 0.175, at most about 0.2, at most about 0.25, at most about 0.3, at most about 0.35, at most about 0.4, at most about 0.45, at most about 0.5, at most about 0.55, at most about 0.6, at most about 0.65, at most about 0.7, at most about 0.75, at most about 0.8, at most about 0.9, at most about 1.0, at most about 1.25, at most about 1.5, at most about 1.75, at most about 2.0, at most about 2.5, at most about 5.0, at most about 7.5, or at most about 10.0 mg/L.

In some embodiments, the working fluid in each reactor described herein may be recirculated (e.g., recycled) within the reactor of the bioreactor system. In some embodiments, the rate of recirculation within the reactor may be between 4-9 gallons/min. In some embodiments, the rate of recirculation within the reactor may be at least about 0.5 gallon/min, at least about 1 gallon/min, at least about 2 gallons/min, at least about 3 gallons/min, at least about 3.5 gallons/min, at least about 4 gallons/min, at least about 4.5 gallons/min, at least about 5 gallons/min, at least about 5.5 gallons/min, at least about 6 gallons/min, at least about 6.5 gallons/min, at least about 7 gallons/min, at least about 7.5 gallons/min, at least about 8 gallons/min, at least about 8.5 gallons/min, at least about 9 gallons/min, at least about 10 gallons/min, at least about 11 gallons/min, at least about 13 gallons/min, at least about 15 gallons/min, at least about 20 gallons/min, at least about 25 gallons/min, at least about 30 gallons/min, at least about 40 gallons/min, or at least about 50 gallons/min.

In some embodiments, the rate of recirculation within the reactor may be at most about 50 gallons/min, at most about 40 gallons/min, at most about 30 gallons/min, at most about 25 gallons/min, at most about 20 gallons/min, at most about 15 gallons/min, at most about 13 gallons/min, at most about 10 gallons/min, at most about 9 gallons/min, at most about 8 gallons/min, at most about 7.5 gallons/min, at most about 7 gallons/min, at most about 6.5 gallons/min, at most about 6 gallons/min, at most about 5.5 gallons/min, at most about 5 gallons/min, at most about 4.5 gallons/min, at most about 4 gallons/min, at most about 3.5 gallons/min, at most about 3 gallons/min, at most about 2 gallons/min, at most about 1 gallon/min, or at most about 0.5 gallon/min.

In some embodiments, the rate of recirculation within the reactor may be about 0.5 gallons/min to about 30 gallons/min. In some embodiments, the rate of recirculation within the reactor may be about 0.5 gallons/min to about 1 gallon/min, about 0.5 gallons/min to about 2 gallons/min, about 0.5 gallons/min to about 3 gallons/min, about 0.5 gallons/min to about 4 gallons/min, about 0.5 gallons/min to about 6 gallons/min, about 0.5 gallons/min to about 8 gallons/min, about 0.5 gallons/min to about 9 gallons/min, about 0.5 gallons/min to about 10 gallons/min, about 0.5 gallons/min to about 15 gallons/min, about 0.5 gallons/min to about 20 gallons/min, about 0.5 gallons/min to about 30 gallons/min, about 1 gallon/min to about 2 gallons/min, about 1 gallon/min to about 3 gallons/min, about 1 gallon/min to about 4 gallons/min, about 1 gallon/min to about 6 gallons/min, about 1 gallon/min to about 8 gallons/min, about 1 gallon/min to about 9 gallons/min, about 1 gallon/min to about 10 gallons/min, about 1 gallon/min to about 15 gallons/min, about 1 gallon/min to about 20 gallons/min, about 1 gallon/min to about 30 gallons/min, about 2 gallons/min to about 3 gallons/min, about 2 gallons/min to about 4 gallons/min, about 2 gallons/min to about 6 gallons/min, about 2 gallons/min to about 8 gallons/min, about 2 gallons/min to about 9 gallons/min, about 2 gallons/min to about 10 gallons/min, about 2 gallons/min to about 15 gallons/min, about 2 gallons/min to about 20 gallons/min, about 2 gallons/min to about 30 gallons/min, about 3 gallons/min to about 4 gallons/min, about 3 gallons/min to about 6 gallons/min, about 3 gallons/min to about 8 gallons/min, about 3 gallons/min to about 9 gallons/min, about 3 gallons/min to about 10 gallons/min, about 3 gallons/min to about 15 gallons/min, about 3 gallons/min to about 20 gallons/min, about 3 gallons/min to about 30 gallons/min, about 4 gallons/min to about 6 gallons/min, about 4 gallons/min to about 8 gallons/min, about 4 gallons/min to about 9 gallons/min, about 4 gallons/min to about 10 gallons/min, about 4 gallons/min to about 15 gallons/min, about 4 gallons/min to about 20 gallons/min, about 4 gallons/min to about 30 gallons/min, about 6 gallons/min to about 8 gallons/min, about 6 gallons/min to about 9 gallons/min, about 6 gallons/min to about 10 gallons/min, about 6 gallons/min to about 15 gallons/min, about 6 gallons/min to about 20 gallons/min, about 6 gallons/min to about 30 gallons/min, about 8 gallons/min to about 9 gallons/min, about 8 gallons/min to about 10 gallons/min, about 8 gallons/min to about 15 gallons/min, about 8 gallons/min to about 20 gallons/min, about 8 gallons/min to about 30 gallons/min, about 9 gallons/min to about 10 gallons/min, about 9 gallons/min to about 15 gallons/min, about 9 gallons/min to about 20 gallons/min, about 9 gallons/min to about 30 gallons/min, about 10 gallons/min to about 15 gallons/min, about 10 gallons/min to about 20 gallons/min, about 10 gallons/min to about 30 gallons/min, about 15 gallons/min to about 20 gallons/min, about 15 gallons/min to about 30 gallons/min, or about 20 gallons/min to about 30 gallons/min.

### A. Zinc Solubilizing Systems

One mode of action of products produced in some embodiments may be enhanced zinc uptake comprised in the products. Another mode of action may be increases in soil zinc and uptake of zinc stimulated by microbes and/or microbial metabolites present in the products produced in embodiments described herein. Microbes and metabolites produced in a system described herein may increase the amount of soluble zinc in soil without a plant present. Microbes and metabolites produced in a system described herein may increase the amount of soluble zinc in soil with a plant present. Biostimulant products of the bioreactor systems described herein may act on a plant to create an increased amount of organic acids which may solubilize zinc. Another mode of action may be increases in soil organic zinc (e.g., organically-bound zinc) and mineralization and uptake of organic zinc (e.g., organically-bound zinc) stimulated by microbes and/or microbial metabolites present in the products produced in embodiments described herein. In some cases, a microbial strain may be incubated at a zinc concentration that selectively promotes growth of the microbe. The population of the microbe may be increased. The population of the microbe may be decreased. In an aspect, the present disclosure provides a method, comprising: transferring a feedstock (e.g., aqueous organic feedstock) and an inoculum of a microbe that is capable of promoting zinc solubilization into a first container comprising a volume of a first working fluid. The aqueous organic feedstock may comprise: (i) a first microbial consortium; and/or (ii) digestion products produced by digestion of an organic material by microbes in the first microbial consortium. In some cases, the inoculum may be incubated at a zinc ion concentration that selectively promotes growth of the microbe. The population of the microbe may be increased. The population of the microbe may be decreased.

In an aspect, the present disclosure provides a method, comprising (a) transferring a feedstock (e.g., aqueous organic feedstock) and an inoculum of a microbe that is capable of promoting zinc solubilization into a first container comprising a volume of a first working fluid, wherein the feedstock (e.g., aqueous organic feedstock) comprises: (i) a first microbial consortium; and (ii) digestion products produced by digestion of an organic material by microbes in the first microbial consortium; and (b) incubating the inoculum at a zinc ion concentration that selectively promotes growth of the microbe, thereby increasing the population of the microbe. Incubation of the inoculum of the microbial strain may increase or maintain a population of the microbial strain relative to one or more microbes of the microbial consortium in the working fluid of the reactor of the bioreactor system.

In some embodiments, incubating the zinc-solubilizing microbial strain at a zinc ion concentration may selectively enhance the survival of zinc solubilizing microbes. In some embodiments, incubating the microbial consortium at a zinc ion concentration may increase the proportion of microbes that can tolerate zinc (e.g., zinc solubilizers or zinc-solubilizing microbes). Without wishing to be bound by theory, addition of a selective pressure having a zinc ion concentration may shift a microbial population towards microbes with zinc solubilizing properties.

In some embodiments, the zinc-solubilizing microbial strain (e.g., microbe) is capable of enhancing zinc uptake in plants or promoting soil zinc content and uptake of zinc from soil. In some embodiments, the conditions of the bioreactor system (e.g., pH, nutrients, flow rate, retention time, selective pressure, carbon source, nitrogen source, or any combination thereof) may promote growth of one or more microbes in the first microbial consortium that may be capable of promoting plant growth or zinc uptake in plants, or of generating metabolites capable of promoting plant growth or zinc uptake in plants. In some embodiments, during the incubating the microbe or one or more microbes in the first microbial consortium, metabolites may be produced capable of promoting plant growth and zinc solubilization. The metabolites may have zinc solubilizing capacity and increase an amount of zinc available to plants. The metabolites may enhance the presence of bioavailable zinc ions. An increase in zinc ions following incubation with zinc-solubilizing metabolites may be from zinc solubilization or stimulation of bacteria on a zinc source (e.g., ZnO). The available zinc for plants may promote growth, vigor, or yield of a plant. In some embodiments, the incubating increases a population of one or more microbes in the microbial consortium capable of promoting plant growth. In some embodiments, the feedstock (e.g., aqueous organic feedstock) further comprises an inorganic substrate. In some embodiments, the first microbial consortium further comprises microbes derived from the inorganic substrate. In some embodiments, the inorganic substrate comprises rock phosphate. In some embodiments, the microbe or microbes may be of the species Bacillus safensis, Bacillus amyloliquefaciens, Bacillus megaterium, Pseudomonas spp., Agrobacterium spp., Bacillus circulans, Burkholderia lata, Lysinibacillus spp., Burkholderia latens, Bacillus cereus, B. aryabhattai, Pseudomonas taiwenensis, Acinetobacter spp., Paenibacillus sonchi, Chitinophaga spp., Gluconobacter spp., Klebsiella spp., Serratia spp., Thiobacillus spp, or any combination thereof. In some embodiments, the microbe is the Bacillus safensis strain deposited under ATCC Accession No. PTA-127681 (MS4666), the Bacillus megaterium strain deposited under ATCC Accession No. PTA-127683 (MS4689), or the Bacillus megaterium strain deposited under ATCC Accession No. PTA-127682 (MS4687). In some embodiments, the first working fluid comprises (a) a second microbial consortium derived from the feedstock (e.g., aqueous organic feedstock), and/or (b) digestion products produced by digestion of substances present in the organic feedstock by the first microbial consortium and the microbe(s). In some embodiments, the method further comprises transferring at least a portion of the first working fluid into a second container comprising a second working fluid and incubating the second working fluid in the second container. In some embodiments, the second working fluid comprises (a) a third microbial consortium derived from the first working fluid, and (b) digestion products produced by digestion of substances present in the first working fluid by the third microbial consortium and the microbe. In some embodiments, the amount of the feedstock (e.g., aqueous organic feedstock) transferred into the first container over a time period is equal to the amount of the first working fluid transferred into the second container over the same time period. In some embodiments, the amount of the feedstock (e.g., aqueous organic feedstock) transferred into the first container over a time period is equal to the amount of the first working fluid transferred into the second container over different time periods. In some embodiments, the amount of the feedstock (e.g., aqueous organic feedstock) transferred into the first container over a time period is not equal to the amount of the first working fluid transferred into the second container over the same time period. In some embodiments, the amount of the feedstock (e.g., aqueous organic feedstock) transferred into the first container over a time period is not equal to the amount of the first working fluid transferred into the second container over different time periods. In some embodiments, the volume of the first working fluid in the first container is maintained constant.

In some embodiments, transferring the feedstock (e.g., aqueous organic feedstock) into the first container comprises continuously flowing the feedstock (e.g., aqueous organic feedstock) into the first container at a first flow rate, transferring the portion of the first working fluid into the second container comprises continuously flowing the portion of the first working fluid into the second container at a second flow rate, and the first flow rate and the second flow rate may be equal. In some embodiments, the method further comprises transferring at least a portion of the second working fluid to a third container comprising a third working fluid and incubating the third working fluid in the third container. In some embodiments, the method further comprises transferring at least a portion of the third working fluid into a fourth container comprising a fourth working fluid and incubating the fourth working fluid in the fourth container. In some embodiments, the first working fluid, the second working fluid, the third working fluid, and the fourth working fluid may be maintained at constant volumes. In some embodiments, the first working fluid, the second working fluid, the third working fluid, and the fourth working fluid may not be maintained at constant volumes. In some embodiments, a plant growth promoting product made by the method. In some embodiments, a method of promoting zinc solubilization which increases plant bioavailable zinc, comprises contacting the plant and/or a medium in which the plant is growing with the product.

The digestion process of the zinc solubilizing technology (ZST) may be an anerobic process. The digestion process of a ZST system may incorporate an aerobic phase, a microaerobic phase, or an anaerobic phase in any container of the bioreactor system. The bioreactor system described herein may comprise an aerobic process, a microaerobic process, an anaerobic process, or any combination thereof.

In some embodiments, a serialized set of reaction chambers may be used in a method of producing a biostimulant product, as described herein. In some embodiments, conditions within reactor chambers may be established to selectively promote the production of one or more microbes that have a specific desired plant growth promoting effect.

### ZST 1.0 System

FIG. 1 schematically illustrates an exemplary batch bioreactor system 100 with conditions (e.g., microbes) that produce biostimulant products that may have a multi-modal way of promoting zinc solubilization. The system 100 may comprise at least one reactor 110 and the reactor 110 may be a fluidized bed reactor or a packed bed reactor. Water may act as a hydraulic source. The reactor may comprise an input channel or a series of input channels that flows inputs 130, 140, 150, 160, 170, 180 into the bioreactor system. In some cases, the inputs can be added individually to the reactor 110. In some cases, the inputs may be added in combination with one another into the reactor 110. The inputs, as described herein, may comprise water, a carbon source 130, a nitrogen source 140, micronutrients 150, a microbial product or intermediate product of another bioreactor system (e.g., PST WB 160), a microbial isolate 170, a zinc source 180, or any combination thereof. The inputs may comprise other organic materials. An outflow port (e.g., a product outflow port) may extend from the reactor from which a biostimulant product or digestion product (e.g., base product) may be collected.

The system 100 may comprise a pH sensor, pH controller, or similar mechanic to monitor and/or control the pH of the reactor and/or working fluid within the reactor so that pH is maintained at a threshold. The pH sensor or controller may be automatic. In some embodiments, a pH in the reactor 110 may be between 4.0-9.0. In some embodiments, the pH in the reactor may be at least about 3.0, at least about 3.5, at least about 4.0, at least about 4.5, at least about 5.0, at least about 5.5, at least about 6.0, at least about 6.5, at least about 7.0, at least about 7.5, at least about 8.0, at least about 8.5, at least about 9.0, at least about 9.5, or at least about 10.0. In some embodiments, the pH in the reactor may be at most about 10.0, at most about 9.5, at most about 9.0, at most about 8.5, at most about 8.0, at most about 7.5, at most about 7.0, at most about 6.5, at most about 6.0, at most about 5.5, at most about 5.0, at most about 4.5, at most about 4.0, at most about 3.5, or at most about 3.0. In some embodiments, the pH in the reactor may be from about 2 to about 10. In some embodiments, the pH in the reactor may be from about 2 to about 3, about 2 to about 4, about 2 to about 4.5, about 2 to about 5, about 2 to about 5.5, about 2 to about 6, about 2 to about 6.5, about 2 to about 7, about 2 to about 8, about 2 to about 9, about 2 to about 10, about 3 to about 4, about 3 to about 4.5, about 3 to about 5, about 3 to about 5.5, about 3 to about 6, about 3 to about 6.5, about 3 to about 7, about 3 to about 8, about 3 to about 9, about 3 to about 10, about 4 to about 4.5, about 4 to about 5, about 4 to about 5.5, about 4 to about 6, about 4 to about 6.5, about 4 to about 7, about 4 to about 8, about 4 to about 9, about 4 to about 10, about 4.5 to about 5, about 4.5 to about 5.5, about 4.5 to about 6, about 4.5 to about 6.5, about 4.5 to about 7, about 4.5 to about 8, about 4.5 to about 9, about 4.5 to about 10, about 5 to about 5.5, about 5 to about 6, about 5 to about 6.5, about 5 to about 7, about 5 to about 8, about 5 to about 9, about 5 to about 10, about 5.5 to about 6, about 5.5 to about 6.5, about 5.5 to about 7, about 5.5 to about 8, about 5.5 to about 9, about 5.5 to about 10, about 6 to about 6.5, about 6 to about 7, about 6 to about 8, about 6 to about 9, about 6 to about 10, about 6.5 to about 7, about 6.5 to about 8, about 6.5 to about 9, about 6.5 to about 10, about 7 to about 8, about 7 to about 9, about 7 to about 10, about 8 to about 9, about 8 to about 10, or about 9 to about 10.

An outflow port (e.g., a product outflow port) may be positioned on the top, middle, and/or bottom of the reactor 110. Additionally, the reactor may comprise another outflow port 180 for reintroducing working fluid back into the same reactor, and may be pumped back to just below the surface of the same reactor to maintain homogeneous conditions within the working solutions. In this manner, working fluid may be recycled within the bioreactor system 100. For example, working fluid from the reactor 110 may be reintroduced back into the reactor 110. Biosolids (e.g., floc) may be generated through the process. Biosolids may comprise organic materials recovered from the feedstock, waste, wastewater, and/or sludge material of a working fluid of a bioreactor system described herein. In some embodiments, floc can comprise biosolids. In some embodiments, floc may comprise biosolids and/or other solid particles from digestion products of organic materials. In some embodiments, floc can be a flocculated mass of microorganisms, extracellular polymeric substance (EPS) and adsorbed organic and inorganic material. A flocculated mass can comprise an aggregated mass of microorganisms, extracellular polymeric substance (EPS) and adsorbed organic and inorganic material. Suspended particles from the organic substrates of the working fluid may aggregate to form floc. Floc may be produced throughout the fermentation process in the bioreactor system.

In some embodiments, the input composition, as described herein, may comprise a selective pressure or a zinc source. Without wishing to be bound by theory, the selective pressure source (e.g., zinc source) may shift the complex microbial consortia of the bioreactor system 100 to increased zinc solubilizing microbes. The selective pressure source may change the ionic concentration of the working fluid. A shift in the complex microbial consortia may occur to favor microbes of the microbial consortium with zinc-solubilizing properties. In some embodiments, the selective pressure may create a high ionic concentration which selectively inhibits the growth of some microbes within the working fluid, while promoting the growth or survival of other microbes (e.g., an inoculum of a microbe and/or zinc-solubilizing microbes of the microbial consortium) in the working fluid. The zinc solubilizing microbes may comprise microbes that can survive a higher zinc ion concentration in a working fluid caused by addition of the selective pressure. In some embodiments, the inoculum of a microbe may comprise a zinc solubilizing microbe. Addition of the selective pressure to the bioreactor system 100 may promote survival of the inoculum of the microbe. The terms “selective pressure source” and “selective pressure” may be used interchangeably. Addition of a selective pressure source (e.g., zinc source) may increase a concentration of zinc within the working fluid of the bioreactor system 100.

An inoculum of a microbial strain in the bioreactor system 100 may be configured to survive as a population of the microbial strain in the increased concentration of zinc. Survival of a microbial strain can comprise a microbial strain that does not die in the presence of the high zinc concentration. Survival (e.g., maintenance) of a microbial strain can comprise a microbial strain that grows (e.g., increases in number) or does not fluctuate in concentration in the presence of the high zinc concentration. Survival (e.g., maintenance) of a population of the microbial strain can comprise a population of the microbial strain that dies at a slower rate relative to a rate of death of other microbes in the presence of the high zinc concentration. Maintenance of a microbial strain added to a bioreactor system herein can comprise percentage of a concentration of the microbial strain present in the base product (e.g., supernatant) of the system relative to an initial concentration added to the bioreactor system. Survival (e.g., maintenance) of a population of the microbial strain following addition of a selective pressure can comprise at least about 0.01%, at least about 0.05%, at least about 1%, at least about 1.5%, at least about 2%, at least about 2.5%, at least about 3%, at least about 3.5%, at least about 4%, at least about 4.5%, at least about 5%, at least about 10%, at least about 20%, at least about 25%, at least about 30%, at least about 35%, at least about 40%, at least about 45%, at least about 50%, at least about 60%, at least about 70%, or at least about 75% of an initial concentration of a population of the microbial strain present in the base product of the bioreactor system. An initial concentration may comprise a concentration of a population of the microbial strain added into a first container of the bioreactor system. Survival (e.g., maintenance) of a population of the microbial strain following addition of a selective pressure can comprise a concentration of the microbial strain that is at most about 75%, at most about 70%, at most about 60%, at most about 50%, at most about 45%, at most about 40%, at most about 35%, at most about 30%, at most about 25%, at most about 20%, or at most about 10% of a concentration of the microbial strain at the beginning of operating of the bioreactor system. Survival (e.g., maintenance) of a population of the microbial strain following addition of a selective pressure can comprise a concentration of the microbial strain that is from about 5% to about 80% of a concentration of the microbial strain at the beginning of operating of the bioreactor system. Survival (e.g., maintenance) of a population of the microbial strain following addition of a selective pressure can comprise a concentration of the microbial strain that is from about 5% to about 10%, about 5% to about 15%, about 5% to about 20%, about 5% to about 25%, about 5% to about 30%, about 5% to about 35%, about 5% to about 40%, about 5% to about 50%, about 5% to about 60%, about 5% to about 70%, about 5% to about 80%, about 10% to about 15%, about 10% to about 20%, about 10% to about 25%, about 10% to about 30%, about 10% to about 35%, about 10% to about 40%, about 10% to about 50%, about 10% to about 60%, about 10% to about 70%, about 10% to about 80%, about 15% to about 20%, about 15% to about 25%, about 15% to about 30%, about 15% to about 35%, about 15% to about 40%, about 15% to about 50%, about 15% to about 60%, about 15% to about 70%, about 15% to about 80%, about 20% to about 25%, about 20% to about 30%, about 20% to about 35%, about 20% to about 40%, about 20% to about 50%, about 20% to about 60%, about 20% to about 70%, about 20% to about 80%, about 25% to about 30%, about 25% to about 35%, about 25% to about 40%, about 25% to about 50%, about 25% to about 60%, about 25% to about 70%, about 25% to about 80%, about 30% to about 35%, about 30% to about 40%, about 30% to about 50%, about 30% to about 60%, about 30% to about 70%, about 30% to about 80%, about 35% to about 40%, about 35% to about 50%, about 35% to about 60%, about 35% to about 70%, about 35% to about 80%, about 40% to about 50%, about 40% to about 60%, about 40% to about 70%, about 40% to about 80%, about 50% to about 60%, about 50% to about 70%, about 50% to about 80%, about 60% to about 70%, about 60% to about 80%, or about 70% to about 80% of a concentration of the microbial strain at the beginning of operating of the bioreactor system.

One or more microbes of a microbial consortium in the bioreactor system 100 may not be able to survive the increased concentration of zinc. Incubating the working fluid comprising a microbial consortium with a selective pressure source (e.g., zinc source) may raise a zinc ion concentration of a working fluid. The population of the microbial strain may be tolerant to the increased zinc ion concentration. The increased zinc ion concentration may be toxic to a portion of microbes in a microbial consortium. A portion of microbes of the microbial consortium may be tolerant to the increased zinc ion concentration. Incubating the working fluid comprising a microbial consortium with a selective pressure source (e.g., zinc source) may increase or maintain an amount of a population of the microbial strain (e.g., a target isolate or isolated microbe) and decrease other microbes in the bioreactor system 100. Incubating the working fluid comprising a microbial consortium with a selective pressure source (e.g., zinc source) may increase or maintain an amount of zinc-solubilizing microbes in a microbial consortium of the working fluid. A maintained amount of a population of the microbial strain may comprise no change in an amount of a microbe across a duration of time. A maintained inoculum of a microbe may change its amount in a working fluid of a bioreactor system 100 less than 0.1%, less than 0.5%, less than 1%, less than 5%, or less than 10% over a duration of time. The duration of time may comprise at least about 1 hour, 2 hours, 3 hours, 4 hours, 5 hours, 6 hours, 12 hours, 18 hours, 24 hours, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 3 months, 4 months, 5 months, 6 months, 9 months, 12 months, 18 months, 24 months, or greater than about 24 months. The duration of time may comprise at most about 24 months, 18 months, 12 months, 9 months, 6 months, 5 months, 4 months, 3 months, 8 weeks, 7 weeks, 6 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 24 hours, 18 hours, 12 hours, 6 hours, 5 hours, 4 hours, 3 hours, 2 hours, 1 hour, or less than about 1 hour. Without wishing to be bound by theory, the addition of a selective pressure source (e.g., zinc source) may increase a proportion of a population of the microbial strain relative to the microbes or at least a portion of the microbes in a microbial consortium in the bioreactor system 100. Without wishing to be bound by theory, incubating the microbial strain with the selective pressure source (e.g., zinc source) may decrease an amount of a population of the microbial strain less than a decrease of at least a portion of the microbes in a microbial consortium in the bioreactor system 100.

The zinc source may comprise zinc oxide (ZnO) and may be added to the bioreactor system 100 on a first day of a digestion process. In some cases, the zinc source (e.g., ZnO) may be added to the bioreactor system 100 daily. In some cases, the zinc source (e.g., ZnO) may be added to the bioreactor system 100 every 2 days, every 3 days, every 4 days, every 5 days, every 6 days, and/or weekly. In some cases, the zinc source may comprise zinc carbonate, zinc sulfate, calamine ore, or any combination thereof. The zinc source (e.g., zinc oxide) may be added to the reactor at a concentration between 0.1-1.0% w/v. The concentration of the zinc source may be based on the concentration of all components of a working fluid of the system as described herein.

In some embodiments, the zinc source (e.g., zinc oxide) may be added to the reactor at a concentration of at least about 0.1% w/v, at least about 0.05% w/v, at least about 0.1% w/v, at least about 0.2% w/v, at least about 0.3% w/v, at least about 0.4% w/v, at least about 0.5% w/v, at least about 0.6% w/v, at least about 0.7% w/v, at least about 0.8% w/v, at least about 0.9% w/v, at least about 1.0% w/v, at least about 1.1% w/v, at least about 1.2% w/v, at least about 1.3% w/v, at least about 1.4% w/v, or at least about 1.5% w/v. In some embodiments, the zinc source (e.g., zinc oxide) may be added to the reactor at a concentration of at most about 1.5% w/v, at most about 1.4% w/v, at most about 1.3% w/v, at most about 1.2% w/v, at most about 1.1% w/v, at most about 1.0% w/v, at most about 0.9% w/v, at most about 0.8% w/v, at most about 0.7% w/v, at most about 0.6% w/v, at most about 0.5% w/v, at most about 0.4% w/v, at most about 0.3% w/v, at most about 0.2% w/v, at most about 0.1% w/v, or at most about 0.05% w/v.

In some embodiments, the zinc source (e.g., zinc oxide) may be added to the reactor at a concentration of about 0.05% w/v to about 1% w/v. In some embodiments, the zinc source (e.g., zinc oxide) may be added to the reactor at a concentration of about 0.05% w/v to about 0.1% w/v, about 0.05% w/v to about 0.2% w/v, about 0.05% w/v to about 0.3% w/v, about 0.05% w/v to about 0.4% w/v, about 0.05% w/v to about 0.45% w/v, about 0.05% w/v to about 0.5% w/v, about 0.05% w/v to about 0.6% w/v, about 0.05% w/v to about 0.7% w/v, about 0.05% w/v to about 0.8% w/v, about 0.05% w/v to about 0.9% w/v, about 0.05% w/v to about 1% w/v, about 0.1% w/v to about 0.2% w/v, about 0.1% w/v to about 0.3% w/v, about 0.1% w/v to about 0.4% w/v, about 0.1% w/v to about 0.45% w/v, about 0.1% w/v to about 0.5% w/v, about 0.1% w/v to about 0.6% w/v, about 0.1% w/v to about 0.7% w/v, about 0.1% w/v to about 0.8% w/v, about 0.1% w/v to about 0.9% w/v, about 0.1% w/v to about 1% w/v, about 0.2% w/v to about 0.3% w/v, about 0.2% w/v to about 0.4% w/v, about 0.2% w/v to about 0.45% w/v, about 0.2% w/v to about 0.5% w/v, about 0.2% w/v to about 0.6% w/v, about 0.2% w/v to about 0.7% w/v, about 0.2% w/v to about 0.8% w/v, about 0.2% w/v to about 0.9% w/v, about 0.2% w/v to about 1% w/v, about 0.3% w/v to about 0.4% w/v, about 0.3% w/v to about 0.45% w/v, about 0.3% w/v to about 0.5% w/v, about 0.3% w/v to about 0.6% w/v, about 0.3% w/v to about 0.7% w/v, about 0.3% w/v to about 0.8% w/v, about 0.3% w/v to about 0.9% w/v, about 0.3% w/v to about 1% w/v, about 0.4% w/v to about 0.45% w/v, about 0.4% w/v to about 0.5% w/v, about 0.4% w/v to about 0.6% w/v, about 0.4% w/v to about 0.7% w/v, about 0.4% w/v to about 0.8% w/v, about 0.4% w/v to about 0.9% w/v, about 0.4% w/v to about 1% w/v, about 0.45% w/v to about 0.5% w/v, about 0.45% w/v to about 0.6% w/v, about 0.45% w/v to about 0.7% w/v, about 0.45% w/v to about 0.8% w/v, about 0.45% w/v to about 0.9% w/v, about 0.45% w/v to about 1% w/v, about 0.5% w/v to about 0.6% w/v, about 0.5% w/v to about 0.7% w/v, about 0.5% w/v to about 0.8% w/v, about 0.5% w/v to about 0.9% w/v, about 0.5% w/v to about 1% w/v, about 0.6% w/v to about 0.7% w/v, about 0.6% w/v to about 0.8% w/v, about 0.6% w/v to about 0.9% w/v, about 0.6% w/v to about 1% w/v, about 0.7% w/v to about 0.8% w/v, about 0.7% w/v to about 0.9% w/v, about 0.7% w/v to about 1% w/v, about 0.8% w/v to about 0.9% w/v, about 0.8% w/v to about 1% w/v, or about 0.9% w/v to about 1% w/v.

### ZST 1.5/1.5+System

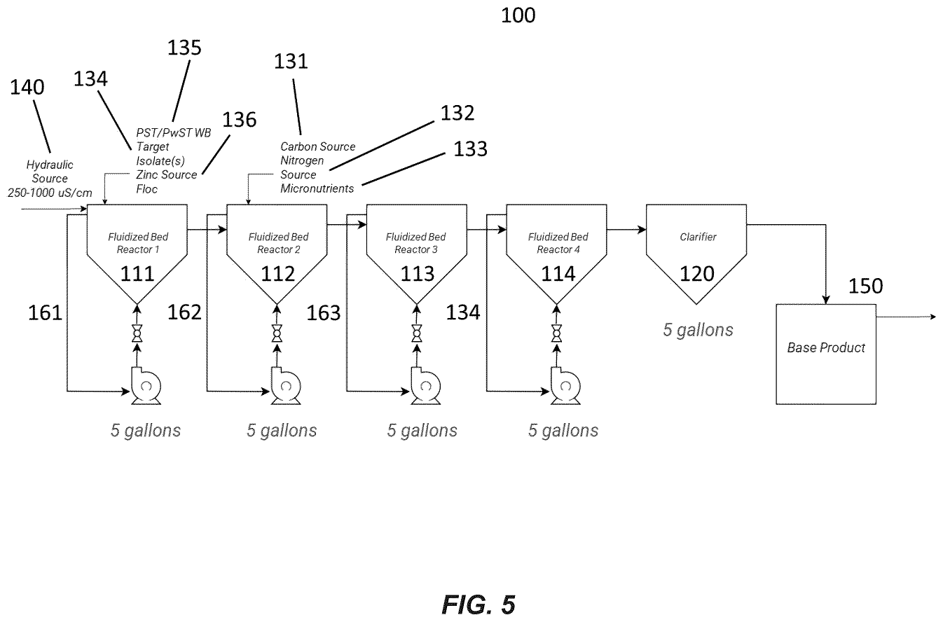

FIG. 5 schematically illustrates an example of a bioreactor system 100 with conditions (e.g., microbes) that produce biostimulant products that may have a multi-modal way of promoting plants to access bound sources of zinc and/or enhance the uptake zinc and other nutrients. The system 100 can comprise a first reactor 111, a second reactor 112, a third reactor 113, a fourth reactor 114, and/or a clarifier container 120 connected sequentially in which feedstock can continuously flow and microbial consortia as described herein can be grown. The first reactor, second reactor, third reactor, and/or fourth reactor may be fluidized bed reactors without the scaffolding or packed bed reactors with a scaffolding within the reactors. Water may act as a hydraulic source 140 and may be coupled to a first reactor 111 and provide continuous flow of water to the reactor. The system 100 may also comprise an input channel or a series of input channels that flows inputs 130 into the bioreactor system. In some cases, the inputs can be added individually to the bioreactor system 100. In some cases, the inputs may be added in combination with one another into the bioreactor system. The inputs, as described herein, may comprise one or more of water, a microbial inoculum 134, a microbial product or intermediate product of another bioreactor system 135, a carbon source 131, a nitrogen source 132, micronutrients 133, and/or a zinc source 136. The inputs may comprise other organic materials. In some cases, the inputs into the bioreactor system may be flown into the first reactor through a water tank. In other cases, the input composition can be added to the first reactor. In other cases, the input composition can be added to the second reactor.

In some embodiments, the input composition, as described herein, may comprise a selective pressure or a zinc source. Without wishing to be bound by theory, the selective pressure source (e.g., zinc source) may shift the complex microbial consortia of the bioreactor system 100 to increased zinc solubilizing microbes. The zinc solubilizing microbes may comprise microbes that can survive a higher zinc ion concentration in a working fluid caused by addition of the selective pressure. In some embodiments, the inoculum of the microbial strain may comprise a zinc solubilizing microbe. Addition of the selective pressure to the bioreactor system 100 may promote survival (e.g., maintenance) of the inoculum of the microbe. The terms “selective pressure source” and “selective pressure” may be used interchangeably. Addition of a selective pressure source (e.g., zinc source) may increase a concentration of zinc within the working fluid of the bioreactor system 100. A population of the microbial strain in the bioreactor system 100 may be configured to survive the increased concentration of zinc. At least a portion of microbes of a microbial consortium in the bioreactor system 100 may not be able to survive the increased concentration of zinc. A shift in the complex microbial consortia may occur to enrich microbes of the microbial consortium with zinc-solubilizing properties. In some embodiments, the selective pressure may create a high ionic concentration which selectively inhibits the growth of some microbes within the working fluid, while promoting the growth or survival of other microbes (e.g., a population of the microbial strain and/or zinc-solubilizing microbes of the microbial consortium) in the working fluid. Addition of a selective pressure source (e.g., zinc source) may increase or maintain an amount of a population of the microbial strain (e.g., a target isolate or isolated microbe) and decrease other microbes in the bioreactor system 100. A maintained amount of a population of the microbial strain may comprise no change in an amount of a microbe across two time periods. A maintained amount of a population of the microbial strain may change its amount in a working fluid of a bioreactor system 100 less than 0.1%, less than 0.5%, less than 1%, less than 5%, or less than 10% over two time periods. Without wishing to be bound by theory, the addition of a selective pressure source (e.g., zinc source) may increase a proportion of a population of the microbial strain relative to the microbes or at least a portion of the microbes in a microbial consortium in the bioreactor system 100. Without wishing to be bound by theory, the addition of a selective pressure source (e.g., zinc source) may decrease an amount of a population of the microbial strain less than a decrease of microbes or at least a portion of the microbes in a microbial consortium in the bioreactor system 100. The zinc source may comprise zinc oxide (ZnO) and may be added to the bioreactor system 100 on a first day of a digestion process. In some cases, the zinc source (e.g., ZnO) may be added to the bioreactor system 100 daily. In some cases, the zinc source (e.g., ZnO) may be added to the bioreactor system 100 every 2 days, every 3 days, every 4 days, every 5 days, every 6 days, and/or weekly. In some cases, the zinc source may comprise one or more of zinc carbonate, zinc sulfate, and/or calamine ore. The zinc source (e.g., zinc oxide) may be added to the first reactor 111, the second reactor 112, or another reactor of the bioreactor system 100. The zinc source (e.g., zinc oxide) may be added to the reactor at a concentration between 0.05-0.5% w/v. A concentration of the zinc source may be based on a total concentration of components of a working fluid of a bioreactor system 100. In some cases, the concentration range of the zinc source (e.g., zinc oxide) in the bioreactor system 100 may be based on the hydraulic retention time of the system. In some embodiments, the zinc source (e.g., zinc oxide) may be added to the reactor at a concentration of at least about 0.01% w/v, at least about 0.02% w/v, at least about 0.03% w/v, at least about 0.04% w/v, at least about 0.05% w/v, at least about 0.1% w/v, at least about 0.2% w/v, at least about 0.3% w/v, at least about 0.4% w/v, at least about 0.5% w/v, at least about 0.6% w/v, at least about 0.7% w/v, at least about 0.8% w/v, at least about 0.9% w/v, at least about 1.0% w/v, at least about 1.1% w/v, at least about 1.2% w/v, at least about 1.3% w/v, at least about 1.4% w/v, or at least about 1.5% w/v. In some embodiments, the zinc source (e.g., zinc oxide) may be added to the reactor at a concentration of at most about 1.5% w/v, at most about 1.4% w/v, at most about 1.3% w/v, at most about 1.2% w/v, at most about 1.1% w/v, at most about 1.0% w/v, at most about 0.9% w/v, at most about 0.8% w/v, at most about 0.7% w/v, at most about 0.6% w/v, at most about 0.5% w/v, at most about 0.4% w/v, at most about 0.3% w/v, at most about 0.2% w/v, at most about 0.1% w/v, at most about 0.05% w/v, at most about 0.04% w/v, at most about 0.03% w/v, at most about 0.02% w/v, or at most about 0.01% w/v.

In some embodiments, the zinc source (e.g., zinc oxide) may be added to the reactor at a concentration of about 0.01% w/v to about 1% w/v. In some embodiments, the zinc source (e.g., zinc oxide) may be added to the reactor at a concentration of about 0.01% w/v to about 0.025% w/v, about 0.01% w/v to about 0.05% w/v, about 0.01% w/v to about 0.075% w/v, about 0.01% w/v to about 0.1% w/v, about 0.01% w/v to about 0.15% w/v, about 0.01% w/v to about 0.2% w/v, about 0.01% w/v to about 0.3% w/v, about 0.01% w/v to about 0.4% w/v, about 0.01% w/v to about 0.5% w/v, about 0.01% w/v to about 0.75% w/v, about 0.01% w/v to about 1% w/v, about 0.025% w/v to about 0.05% w/v, about 0.025% w/v to about 0.075% w/v, about 0.025% w/v to about 0.1% w/v, about 0.025% w/v to about 0.15% w/v, about 0.025% w/v to about 0.2% w/v, about 0.025% w/v to about 0.3% w/v, about 0.025% w/v to about 0.4% w/v, about 0.025% w/v to about 0.5% w/v, about 0.025% w/v to about 0.75% w/v, about 0.025% w/v to about 1% w/v, about 0.05% w/v to about 0.075% w/v, about 0.05% w/v to about 0.1% w/v, about 0.05% w/v to about 0.15% w/v, about 0.05% w/v to about 0.2% w/v, about 0.05% w/v to about 0.3% w/v, about 0.05% w/v to about 0.4% w/v, about 0.05% w/v to about 0.5% w/v, about 0.05% w/v to about 0.75% w/v, about 0.05% w/v to about 1% w/v, about 0.075% w/v to about 0.1% w/v, about 0.075% w/v to about 0.15% w/v, about 0.075% w/v to about 0.2% w/v, about 0.075% w/v to about 0.3% w/v, about 0.075% w/v to about 0.4% w/v, about 0.075% w/v to about 0.5% w/v, about 0.075% w/v to about 0.75% w/v, about 0.075% w/v to about 1% w/v, about 0.1% w/v to about 0.15% w/v, about 0.1% w/v to about 0.2% w/v, about 0.1% w/v to about 0.3% w/v, about 0.1% w/v to about 0.4% w/v, about 0.1% w/v to about 0.5% w/v, about 0.1% w/v to about 0.75% w/v, about 0.1% w/v to about 1% w/v, about 0.15% w/v to about 0.2% w/v, about 0.15% w/v to about 0.3% w/v, about 0.15% w/v to about 0.4% w/v, about 0.15% w/v to about 0.5% w/v, about 0.15% w/v to about 0.75% w/v, about 0.15% w/v to about 1% w/v, about 0.2% w/v to about 0.3% w/v, about 0.2% w/v to about 0.4% w/v, about 0.2% w/v to about 0.5% w/v, about 0.2% w/v to about 0.75% w/v, about 0.2% w/v to about 1% w/v, about 0.3% w/v to about 0.4% w/v, about 0.3% w/v to about 0.5% w/v, about 0.3% w/v to about 0.75% w/v, about 0.3% w/v to about 1% w/v, about 0.4% w/v to about 0.5% w/v, about 0.4% w/v to about 0.75% w/v, about 0.4% w/v to about 1% w/v, about 0.5% w/v to about 0.75% w/v, about 0.5% w/v to about 1% w/v, or about 0.75% w/v to about 1% w/v.

### B. Phosphate Solubilizing Systems

One mode of action of products produced in some embodiments is enhanced phosphate solubilization comprised in the products. Microbes and metabolites produced in a system described herein may increase the amount of soluble phosphate in soil without a plant present. Microbes and metabolites produced in a system described herein may increase the amount of soluble phosphate in soil with a plant present. Biostimulant products of the bioreactor systems described herein may act on a plant to create an increased amount of organic acids which may solubilize phosphates. Another mode of action may be increases in soil organic phosphate (e.g., organically-bound phosphate) and mineralization and uptake of organic phosphate (e.g., organically-bound phosphate) stimulated by microbes and/or microbial metabolites present in the products produced in embodiments described herein. In some cases, a microbial strain may be incubated at a phosphate (e.g., phosphorus) concentration that selectively promotes growth of the microbe. The population of the microbe may be increased. The population of the microbe may be decreased.

In an aspect, the present disclosure provides a method comprising transferring a feedstock (e.g., aqueous organic feedstock) and/or an inoculum of a phosphate-solubilizing microbe into a first container. In some embodiments, the first container may comprise a volume of a first working fluid. The feedstock (e.g., aqueous organic feedstock) may comprise a first microbial consortium, digestion products, or any combination thereof. The digestion products may be generated by digestion of organic material by microbes of the first microbial consortium. The method may further comprise incubating a population of the microbial strain in a selective pressure source. The selective pressure source may comprise insoluble phosphate. Incubating the working fluid comprising the phosphate solubilizing microbial strain and feedstock (e.g., organic feedstock) with the selective pressure may promote enrichment of phosphate-solubilizing microbes within the microbial consortium, the inoculated phosphate microbial strain, generated phosphate-solubilizing metabolites, or any combination thereof. Incubating the working fluid comprising the phosphate solubilizing microbial strain and feedstock (e.g., organic feedstock) with the selective pressure may promote maintenance of phosphate-solubilizing microbes within the microbial consortium, the inoculated phosphate microbial strain, generated phosphate-solubilizing metabolites, or any combination thereof. Incubation of the phosphate-solubilizing microbial strain in the bioreactor system may increase or maintain a proportion of phosphate-solubilizing microbes (e.g., phosphate solubilizers) relative to a total bacteria count within a container of the bioreactor system. Incubation of the phosphate-solubilizing microbial strain in the bioreactor system may increase or maintain a proportion of phosphate-solubilizing microbes (e.g., phosphate solubilizers) relative to a total bacteria count across two or more containers (e.g., a first container, a second container, a third container, a fourth container, a fifth container, a sixth container, or any combination thereof) of the bioreactor system.

In an aspect, the present disclosure provides a method, comprising (a) transferring a feedstock (e.g., aqueous organic feedstock) and an inoculum of a microbe that is capable of promoting phosphate solubilization into a first container comprising a volume of a first working fluid, wherein the feedstock (e.g., aqueous organic feedstock) comprises: (i) a first microbial consortium; and (ii) digestion products produced by digestion of an organic material by microbes in the first microbial consortium; and (b) incubating a population of the microbial strain with a selective pressure comprising insoluble phosphate that selectively promotes growth of the microbe, thereby increasing the population of the microbe. Incubation of the inoculum of the microbe may increase or maintain a proportion of the inoculum of the microbe relative to microbe of the microbial consortium in the working fluid of the reactor of the bioreactor system.

Nutrients (e.g., macronutrients, micronutrients, inorganic nutrients, or any combination thereof) can be present in the digestion system to provide an environment for bacterial growth. Macronutrients (phosphorus, carbon, nitrogen, or any combination thereof) may help enrich a population of microbes with a target functionality (e.g., phosphate solubilization activity). Limiting an essential macronutrient such as phosphorous can act as a selective pressure which can increase the activity of phosphate solubilizing microorganisms. The addition of a selective pressure (e.g., an insoluble phosphate source such as rock phosphate) with inputs to facilitate microbial growth can allow for enrichment of phosphate solubilizing activity in the working fluid of a digestion system described herein. The phosphate solubilizers can comprise the inoculum of the microbe (e.g., a population of the inoculum of the microbe), phosphate-solubilizing microbes of a microbial consortium, phosphate-solubilizing metabolites produced by the inoculum of the microbe, or any combination thereof. Without wishing to be bound by theory, addition of a selective pressure having a phosphate ion concentration may shift a microbial population towards microbes with phosphate solubilizing properties. In some cases, presence of a selective pressure (e.g., rock phosphate particles) may increase a phosphate solubilization activity of a microbial community. The phosphate solubilization activity of the community may increase while a number of culturable phosphate solubilizers (e.g., phosphate-solubilizing microbes) does not increase or decrease. The phosphate solubilization activity of the community may increase while a number of culturable phosphate solubilizers (e.g., phosphate-solubilizing microbes) may increase or decrease. In some embodiments, incubating the inoculum at a phosphate ion concentration caused by addition of a selective pressure may increase an amount of an inoculum of the microbe. Without wishing to be bound by theory, addition of a selective pressure having a ion concentration (e.g., phosphate ion concentration or zinc ion concentration) may shift a microbial population towards microbes with one or more particular solubilizing properties. In some embodiments, a mode of action for enrichment of one or more microbes comprising a selective pressure mechanism as described herein may apply to any other ionic species, organic and/or inorganic substrates, or any combination thereof to enrich a microbial population in a working fluid (e.g., a target microbial population).

In some embodiments, the microbe is capable of phosphate solubilization in plants or promoting soil organic phosphate content and mineralization and uptake of organic phosphate (e.g., organically-bound phosphate) from soil. In some embodiments, the conditions of the bioreactor system (e.g., pH, nutrients, flow rate, retention time, selective pressure, carbon source, nitrogen source, or any combination thereof) may promote growth of one or more microbes in the first microbial consortium that may be capable of promoting plant growth or phosphate solubilization in plants, or of generating metabolites capable of promoting plant growth or phosphate solubilization in plants. In some embodiments, during the incubating the microbe or one or more microbes in the first microbial consortium, metabolites may be produced capable of promoting plant growth and phosphate solubilization. The metabolites may have phosphate solubilizing capacity and make phosphate available to plants. The metabolites may enhance the presence of bioavailable phosphate ions. An increase in phosphate ions following incubation with phosphate-solubilizing metabolites may be from phosphate solubilization or stimulation of bacteria on a source of insoluble phosphate (e.g., rock phosphate). The available phosphate for plants may promote growth, vigor, or yield of a plant. In some embodiments, the incubating increases a population of one or more microbes in the microbial consortium capable of promoting plant growth. In some embodiments, the feedstock (e.g., aqueous organic feedstock) further comprises an inorganic substrate. In some embodiments, the first microbial consortium further comprises microbes derived from the inorganic substrate. In some embodiments, the inorganic substrate comprises rock phosphate. In some embodiments, the microbe or microbes may be of the species Bacillus safensis, Bacillus amyloliquefaciens, Bacillus licheniformis, Bacillus megaterium, Commonas testosteroni, Enterobacter cloacae, Citrabacter freundii, Pseudomonas spp., Agrobacterium spp., Bacillus circulans, or any combination thereof. In some embodiments, the microbe is the Bacillus amyloliquefaciens strain deposited under Accession No. PTA-127657 (MS2839) or the Bacillus licheniformis strain deposited under Accession No. PTA-127656 (MS1835). In some embodiments, the first working fluid comprises (a) a second microbial consortium derived from the feedstock (e.g., aqueous organic feedstock), and/or (b) digestion products produced by digestion of substances present in the organic feedstock by the first microbial consortium and the microbe(s). In some embodiments, the method further comprises transferring a portion of the first working fluid into a second container comprising a second working fluid and incubating the second working fluid in the second container. In some embodiments, the second working fluid comprises (a) a third microbial consortium derived from the first working fluid, and (b) digestion products produced by digestion of substances present in the first working fluid by the third microbial consortium and the microbe. In some embodiments, the amount of the feedstock (e.g., aqueous organic feedstock) transferred into the first container over a time period is equal to the amount of the first working fluid transferred into the second container over the same time period. In some embodiments, the amount of the feedstock (e.g., aqueous organic feedstock) transferred into the first container over a time period is different to the amount of the first working fluid transferred into the second container over the same time period. In some embodiments, the amount of the feedstock (e.g., aqueous organic feedstock) transferred into the first container over a time period is more compared to the amount of the first working fluid transferred into the second container over the same time period. In some embodiments, the amount of the feedstock (e.g., aqueous organic feedstock) transferred into the first container over a time period is less compared to the amount of the first working fluid transferred into the second container over the same time period. In some embodiments, the volume of the first working fluid in the first container is maintained constant. A constant volume of working fluid in the first container may comprise the same volume of working fluid over 1 minute, 2 minutes, 3 minutes, 4 minutes, 5 minutes, 10 minutes, 15 minutes, 20 minutes, 30 minutes, 45 minutes, 1 hour, 2 hours, 3 hours, 4 hours, 5 hours, 10 hours, 12 hours, 18 hours, 1 day, 2 days, 3 days, 4 days, 5 days, 6 days, 1 week, 2 weeks, 3 weeks, 1 month, 3 months, 6 months, or 1 year. In some embodiments, the volume of the first working fluid in the first container is different over time. In some embodiments, transferring the feedstock (e.g., aqueous organic feedstock) into the first container comprises continuously flowing the feedstock (e.g., aqueous organic feedstock) into the first container at a first flow rate, transferring the portion of the first working fluid into the second container comprises continuously flowing the portion of the first working fluid into the second container at a second flow rate, and the first flow rate and the second flow rate may be equal. In some embodiments, the method further comprises transferring a portion of the second working fluid to a third container comprising a third working fluid and incubating the third working fluid in the third container. In some embodiments, the method further comprises transferring a portion of the third working fluid into a fourth container comprising a fourth working fluid and incubating the fourth working fluid in the fourth container. In some embodiments, the first working fluid, the second working fluid, the third working fluid, and the fourth working fluid may be maintained at constant volumes. In some embodiments, a plant growth promoting product made by the method. In some embodiments, a method of promoting phosphate solubilization of a plant comprising contacting the plant and/or a medium in which the plant is growing with the product.

In some embodiments, a serialized set of reaction chambers may be used in a method of producing a biostimulant product, as described in more detail in the Examples below. In some embodiments, conditions within reactor chambers may be established to selectively promote the production of one or more microbes that have a specific desired plant growth promoting effect.

In some embodiments, the input composition, as described herein, may comprise a selective pressure or a phosphate source (e.g., phosphate ion source) 140. Without wishing to be bound by theory, the selective pressure source (e.g., phosphate source) may shift the complex microbial consortia of the bioreactor system 100 to increased phosphate solubilizing microbes. The selective pressure source can comprise an insoluble phosphate source. The selective pressure source may change the ionic concentration of the working fluid. Microbes of the microbial consortia may not be able to live in the new ionic environment and a shift in the complex microbial consortia may occur. In some embodiments, the selective pressure may create a high ionic concentration which selectively increases a solubilizing activity (e.g., a phosphate solubilizing activity) of some microbes within the working fluid. Upon introduction of the selective pressure (e.g., insoluble phosphate source), microbes capable of phosphate solubilization (e.g., phosphate solubilizers) may solubilize the insoluble phosphate. The phosphate solubilizers may become enriched in the working fluid (e.g., become an enriched microbial strain). In some cases, the phosphate solubilizers may produce metabolites. The metabolites may be capable of phosphate solubilization (e.g., solubilizing the insoluble phosphate). In some embodiments, the inoculum of a microbe may comprise a phosphate solubilizing microbe. Addition of the selective pressure to the bioreactor system 100 may promote survival of the inoculum of the microbe. The terms “selective pressure source” and “selective pressure” may be used interchangeably. Addition of a selective pressure source (e.g., phosphate source) may increase a concentration of phosphate within the working fluid of the bioreactor system 100.

In some embodiments, the sole or primary source of insoluble phosphate in a bioreactor system may comprise rock phosphate. In some embodiments, the sole or primary source of phosphate may comprise different rock phosphates from different sources that may contain different compositions of phosphorous and/or other bound micronutrients, macronutrients, or metals. In some embodiments, the sole or primary source of phosphate may comprise rock phosphate mined from different geographies. A sole or primary source of phosphate may be added to enrich for a phosphate-solubilizing community of microbes and/or metabolites and/or to stimulate phosphate-solubilizing activity by microbes within the system. The sole or primary source of phosphate (e.g., rock phosphate) may be added to the bioreactor system 100 on a first day of a digestion process. The sole or primary source of phosphate (e.g., rock phosphate) may be added to the first reactor 110, the second reactor 115, or another reactor of the bioreactor system. A source of insoluble phosphate (e.g., rock phosphate) may be added into the first reactor at a range of 10.0-50.0 grams per day, based on the span of the hydraulic retention time of the system. A hydraulic retention time may comprise an amount of time a working fluid is maintained in a container (e.g., is not transferred out) of a bioreactor system as described herein. In some embodiments, a selective pressure (e.g., rock phosphate) may be added to the bioreactor system at a concentration of at least about 1 gram/day, at least about 2 grams/day, at least about 5 grams/day, at least about 7 grams/day, at least about 10 grams/day, at least about 15 grams/day, at least about 20 grams/day, at least about 25 grams/day, at least about 30 grams/day, at least about 35 grams/day, at least about 40 grams/day, at least about 45 grams/day, at least about 50 grams/day, at least about 55 grams/day, at least about 60 grams/day, at least about 70 grams/day, at least about 80 grams/day, at least about 90 grams/day, or at least about 100 grams/day. In some embodiments, rock phosphate may be added to the bioreactor system at a concentration of at most about 100 grams/day, at most about 90 grams/day, at most about 80 grams/day, at most about 70 grams/day, at most about 60 grams/day, at most about 55 grams/day, at most about 50 grams/day, at most about 45 grams/day, at most about 40 grams/day, at most about 35 grams/day, at most about 30 grams/day, at most about 25 grams/day, at most about 20 grams/day, at most about 15 grams/day, at most about 10 grams/day, at most about 7 grams/day, at most about 5 grams/day, at most about 2 grams/day, or at most about 1 gram/day.

In some embodiments, a source of insoluble phosphate (e.g., rock phosphate) may be added to the bioreactor system at a concentration of about 5 grams/day to about 100 grams/day. In some embodiments, a source of insoluble phosphate (e.g., rock phosphate) may be added to the bioreactor system at a concentration of about 5 grams/day to about 10 grams/day, about 5 grams/day to about 15 grams/day, about 5 grams/day to about 20 grams/day, about 5 grams/day to about 25 grams/day, about 5 grams/day to about 30 grams/day, about 5 grams/day to about 35 grams/day, about 5 grams/day to about 40 grams/day, about 5 grams/day to about 50 grams/day, about 5 grams/day to about 60 grams/day, about 5 grams/day to about 75 grams/day, about 5 grams/day to about 100 grams/day, about 10 grams/day to about 15 grams/day, about 10 grams/day to about 20 grams/day, about 10 grams/day to about 25 grams/day, about 10 grams/day to about 30 grams/day, about 10 grams/day to about 35 grams/day, about 10 grams/day to about 40 grams/day, about 10 grams/day to about 50 grams/day, about 10 grams/day to about 60 grams/day, about 10 grams/day to about 75 grams/day, about 10 grams/day to about 100 grams/day, about 15 grams/day to about 20 grams/day, about 15 grams/day to about 25 grams/day, about 15 grams/day to about 30 grams/day, about 15 grams/day to about 35 grams/day, about 15 grams/day to about 40 grams/day, about 15 grams/day to about 50 grams/day, about 15 grams/day to about 60 grams/day, about 15 grams/day to about 75 grams/day, about 15 grams/day to about 100 grams/day, about 20 grams/day to about 25 grams/day, about 20 grams/day to about 30 grams/day, about 20 grams/day to about 35 grams/day, about 20 grams/day to about 40 grams/day, about 20 grams/day to about 50 grams/day, about 20 grams/day to about 60 grams/day, about 20 grams/day to about 75 grams/day, about 20 grams/day to about 100 grams/day, about 25 grams/day to about 30 grams/day, about 25 grams/day to about 35 grams/day, about 25 grams/day to about 40 grams/day, about 25 grams/day to about 50 grams/day, about 25 grams/day to about 60 grams/day, about 25 grams/day to about 75 grams/day, about 25 grams/day to about 100 grams/day, about 30 grams/day to about 35 grams/day, about 30 grams/day to about 40 grams/day, about 30 grams/day to about 50 grams/day, about 30 grams/day to about 60 grams/day, about 30 grams/day to about 75 grams/day, about 30 grams/day to about 100 grams/day, about 35 grams/day to about 40 grams/day, about 35 grams/day to about 50 grams/day, about 35 grams/day to about 60 grams/day, about 35 grams/day to about 75 grams/day, about 35 grams/day to about 100 grams/day, about 40 grams/day to about 50 grams/day, about 40 grams/day to about 60 grams/day, about 40 grams/day to about 75 grams/day, about 40 grams/day to about 100 grams/day, about 50 grams/day to about 60 grams/day, about 50 grams/day to about 75 grams/day, about 50 grams/day to about 100 grams/day, about 60 grams/day to about 75 grams/day, about 60 grams/day to about 100 grams/day, or about 75 grams/day to about 100 grams/day.

### C. Nitrogen Use Efficiency System

One mode of action of products produced in some embodiments is enhanced nitrogen use efficiency comprised in the products. Microbes and metabolites produced in a system described herein may increase nitrogen use efficiency in soil without a plant present. Microbes and metabolites produced in a system described herein may increase nitrogen use efficiency in soil with a plant present. Biostimulant products of the bioreactor systems described herein may act on a plant to create an increased amount of organic acids which may improve nitrogen use efficiency. Another mode of action may be increases in soil nitrogen fixation stimulated by microbes and/or microbial metabolites present in the products produced in embodiments described herein.

In some embodiments, the supernatant (or base product) from the clarifier container may be continuously collected, and a portion of the floc at the bottom of the clarifier can be returned to the first reactor at a rate of recirculation of about 0.5 L/day to about 15 L/day. In some embodiments, the supernatant (or base product) from the clarifier container may be continuously collected, and a portion of the floc at the bottom of the clarifier can be returned to the first reactor at a rate of recirculation of about 0.5 L/day to about 1 L/day, about 0.5 L/day to about 1.5 L/day, about 0.5 L/day to about 2 L/day, about 0.5 L/day to about 3 L/day, about 0.5 L/day to about 4 L/day, about 0.5 L/day to about 4.5 L/day, about 0.5 L/day to about 5 L/day, about 0.5 L/day to about 7.5 L/day, about 0.5 L/day to about 10 L/day, about 0.5 L/day to about 12 L/day, about 0.5 L/day to about 15 L/day, about 1 L/day to about 1.5 L/day, about 1 L/day to about 2 L/day, about 1 L/day to about 3 L/day, about 1 L/day to about 4 L/day, about 1 L/day to about 4.5 L/day, about 1 L/day to about 5 L/day, about 1 L/day to about 7.5 L/day, about 1 L/day to about 10 L/day, about 1 L/day to about 12 L/day, about 1 L/day to about 15 L/day, about 1.5 L/day to about 2 L/day, about 1.5 L/day to about 3 L/day, about 1.5 L/day to about 4 L/day, about 1.5 L/day to about 4.5 L/day, about 1.5 L/day to about 5 L/day, about 1.5 L/day to about 7.5 L/day, about 1.5 L/day to about 10 L/day, about 1.5 L/day to about 12 L/day, about 1.5 L/day to about 15 L/day, about 2 L/day to about 3 L/day, about 2 L/day to about 4 L/day, about 2 L/day to about 4.5 L/day, about 2 L/day to about 5 L/day, about 2 L/day to about 7.5 L/day, about 2 L/day to about 10 L/day, about 2 L/day to about 12 L/day, about 2 L/day to about 15 L/day, about 3 L/day to about 4 L/day, about 3 L/day to about 4.5 L/day, about 3 L/day to about 5 L/day, about 3 L/day to about 7.5 L/day, about 3 L/day to about 10 L/day, about 3 L/day to about 12 L/day, about 3 L/day to about 15 L/day, about 4 L/day to about 4.5 L/day, about 4 L/day to about 5 L/day, about 4 L/day to about 7.5 L/day, about 4 L/day to about 10 L/day, about 4 L/day to about 12 L/day, about 4 L/day to about 15 L/day, about 4.5 L/day to about 5 L/day, about 4.5 L/day to about 7.5 L/day, about 4.5 L/day to about 10 L/day, about 4.5 L/day to about 12 L/day, about 4.5 L/day to about 15 L/day, about 5 L/day to about 7.5 L/day, about 5 L/day to about 10 L/day, about 5 L/day to about 12 L/day, about 5 L/day to about 15 L/day, about 7.5 L/day to about 10 L/day, about 7.5 L/day to about 12 L/day, about 7.5 L/day to about 15 L/day, about 10 L/day to about 12 L/day, about 10 L/day to about 15 L/day, or about 12 L/day to about 15 L/day.

In some embodiments, the supernatant from the clarifier container may be continuously collected, and a portion of the floc at the bottom of the clarifier can be returned to the first reactor at a rate of recirculation of at least about 10 L/day, at least about 50 L/day, at least about 100 L/day, at least about 200 L/day, at least about 300 L/day, at least about 400 L/day, at least about 500 L/day, at least about 750 L/day, at least about 1000 L/day, at least about 2500 L/day, at least about 5000 L/day, or greater than about 5000 L/day. In some embodiments, the supernatant from the clarifier container may be continuously collected, and a portion of the floc at the bottom of the clarifier can be returned to the first reactor at a rate of recirculation of at most about 5000 L/day, at most about 2500 L/day, at most about 1000 L/day, at most about 750 L/day, at most about 500 L/day, at most about 400 L/day, at most about 300 L/day, at most about 200 L/day, at most about 100 L/day, at most about 50 L/day, at most about 10 L/day, or less than about 10 L/day.

In some embodiments, the supernatant from the clarifier container may be continuously collected, and a portion of the floc at the bottom of the clarifier can be returned to the first reactor at a rate of recirculation between about 10 L/day to about 5,000 L/day. In some embodiments, the supernatant from the clarifier container may be continuously collected, and a portion of the floc at the bottom of the clarifier can be returned to the first reactor at a rate of recirculation between about 10 L/day to about 25 L/day, about 10 L/day to about 50 L/day, about 10 L/day to about 100 L/day, about 10 L/day to about 250 L/day, about 10 L/day to about 500 L/day, about 10 L/day to about 750 L/day, about 10 L/day to about 1,000 L/day, about 10 L/day to about 2,000 L/day, about 10 L/day to about 3,000 L/day, about 10 L/day to about 4,000 L/day, about 10 L/day to about 5,000 L/day, about 25 L/day to about 50 L/day, about 25 L/day to about 100 L/day, about 25 L/day to about 250 L/day, about 25 L/day to about 500 L/day, about 25 L/day to about 750 L/day, about 25 L/day to about 1,000 L/day, about 25 L/day to about 2,000 L/day, about 25 L/day to about 3,000 L/day, about 25 L/day to about 4,000 L/day, about 25 L/day to about 5,000 L/day, about 50 L/day to about 100 L/day, about 50 L/day to about 250 L/day, about 50 L/day to about 500 L/day, about 50 L/day to about 750 L/day, about 50 L/day to about 1,000 L/day, about 50 L/day to about 2,000 L/day, about 50 L/day to about 3,000 L/day, about 50 L/day to about 4,000 L/day, about 50 L/day to about 5,000 L/day, about 100 L/day to about 250 L/day, about 100 L/day to about 500 L/day, about 100 L/day to about 750 L/day, about 100 L/day to about 1,000 L/day, about 100 L/day to about 2,000 L/day, about 100 L/day to about 3,000 L/day, about 100 L/day to about 4,000 L/day, about 100 L/day to about 5,000 L/day, about 250 L/day to about 500 L/day, about 250 L/day to about 750 L/day, about 250 L/day to about 1,000 L/day, about 250 L/day to about 2,000 L/day, about 250 L/day to about 3,000 L/day, about 250 L/day to about 4,000 L/day, about 250 L/day to about 5,000 L/day, about 500 L/day to about 750 L/day, about 500 L/day to about 1,000 L/day, about 500 L/day to about 2,000 L/day, about 500 L/day to about 3,000 L/day, about 500 L/day to about 4,000 L/day, about 500 L/day to about 5,000 L/day, about 750 L/day to about 1,000 L/day, about 750 L/day to about 2,000 L/day, about 750 L/day to about 3,000 L/day, about 750 L/day to about 4,000 L/day, about 750 L/day to about 5,000 L/day, about 1,000 L/day to about 2,000 L/day, about 1,000 L/day to about 3,000 L/day, about 1,000 L/day to about 4,000 L/day, about 1,000 L/day to about 5,000 L/day, about 2,000 L/day to about 3,000 L/day, about 2,000 L/day to about 4,000 L/day, about 2,000 L/day to about 5,000 L/day, about 3,000 L/day to about 4,000 L/day, about 3,000 L/day to about 5,000 L/day, or about 4,000 L/day to about 5,000 L/day.

In some embodiments, the input composition, as described herein, may comprise a carbon source. The carbon source may be glucose, malate, lactose, sucrose, pyruvate, other simple sugars, or any combination thereof. The carbon source (e.g., glucose, malate) may be added to the first reactor to maintain a concentration of carbon source in the working fluid of 0.2%-2.0% w/v based on the span of the hydraulic retention time of the bioreactor system. In some embodiments, the carbon source (e.g., glucose, malate) may be added to the first reactor to maintain a concentration of carbon source in the working fluid of a carbon source in working fluid of at least about 0.1%, 0.25%, 0.5%, 0.75%, 1.0%, 1.25%, 1.5%, 1.75%, 2.0%, 2.5%, 3.0%, 5.0%, 7.5%, or 10.0% w/v. In some embodiments, the carbon source (e.g., glucose, malate) may be added to the first reactor (e.g., added to a first working fluid) to maintain a concentration of carbon source in the working fluid of at most about 10.0%, 7.5%, 5.0%, 3.0%, 2.5%, 2.0%, 1.75%, 1.5%, 1.25%, 1.0%, 0.75%, 0.5%, 0.25%, or 0.1% w/v.

In some embodiments, the input composition, as described herein, may comprise a nitrogen source. The nitrogen source may be ammonium sulfate, ammonium chloride, ammonium nitrate, sodium nitrate, yeast extract, yeast, or any combination thereof. The nitrogen source (e.g., ammonium sulfate) may be added to the first reactor to maintain a concentration of nitrogen source in the working fluid of 0.02-0.2% w/v based on the span of the hydraulic retention time of the bioreactor system. In some embodiments, the nitrogen source (e.g., ammonium sulfate) may be added to the first reactor to maintain a concentration of nitrogen source in the working fluid of a nitrogen source in a working fluid of at least about 0.005%, 0.01%, 0.015%, 0.02%, 0.025%, 0.03%, 0.035%, 0.04%, 0.045%, 0.05%, 0.055%, 0.06%, 0.065%, 0.07%, 0.075%, 0.1%, 0.125%, 0.15%, 0.175%, 0.2%, 0.25%, 0.3%, 0.4%, 0.5%, 0.75%, 1.0%, 1.5%, 2.0%, 2.5%, or 3.0% w/v. In some embodiments, the nitrogen source (e.g., ammonium sulfate) may be added to the first reactor to maintain a concentration of nitrogen source in the working fluid of at most about 3.0% 2.5%, 2.0%, 1.5%, 1.0%, 0.75%, 0.5%, 0.4%, 0.3%, 0.25%, 0.2%, 0.175%, 0.15%, 0.125%, 0.1%, 0.075%, 0.07%, 0.065%, 0.06%, 0.055%, 0.05%, 0.045%, 0.04%, 0.035%, 0.03%, 0.025%, 0.02%, 0.015%, 0.01%, 0.005% w/v.

In some embodiments, a concentration range of 0.5-4.5% v/v of Whole Broth of a Phosphate Solubilization Technology, PST WB (in a ratio of 1:3 floc: supernatant (SPN)) or the Whole Broth of a Water Based Phosphate Solubilization Technology, PwST WB (in a ratio of 1:3 floc: SPN) may also be added to the first reactor on the span of the hydraulic retention time of the bioreactor system. The floc: SPN may be a combined mixture of floc (e.g., biosolids) and supernatant from the clarifier. In some embodiments, the concentration range of PST WB or PwST WB in a working fluid of a reactor may be at least about 0.05% v/v, 0.1% v/v, 0.2% v/v, 0.3% v/v, 0.4% v/v, 0.5% v/v, 0.6% v/v, 0.7% v/v, 0.8% v/v, 0.9% v/v, 1.0% v/v, 1.5% v/v, 2.0% v/v, 2.5% v/v, 3.0% v/v, 3.5% v/v, 4.0% v/v, 4.5% v/v, 5.0% v/v, 5.5% v/v, 6.0% v/v, 6.5% v/v, 7.0% v/v, 7.5% v/v, 10.0% v/v, 12.5% v/v, 15.0% v/v, or greater than about 15.0%. In some embodiments, the concentration range of PST WB or PwST WB in a working fluid of a reactor may be at most about 15.0% v/v, 12.5% v/v, 10.0% v/v, 7.5% v/v, 7.0% v/v, 6.5% v/v, 6.0% v/v, 5.5% v/v, 5.0% v/v, 4.5% v/v, 4.0% v/v, 3.5% v/v, 3.0% v/v, 2.5% v/v, 2.0% v/v, 1.5% v/v, 1.0% v/v, 0.9% v/v, 0.8% v/v, 0.7% v/v, 0.6% v/v, 0.5% v/v, 0.4% v/v, 0.3% v/v, 0.2% v/v, 0.1% v/v, 0.05% v/v, or less than about 0.05% v/v.

In some embodiments, the bioreactor system may be inoculated with isolates MS3900 and/or MS3907. In some embodiments, the bioreactor system may be inoculated with isolates MS3900, MS3907, MS4921, and/or any combination thereof.

In some embodiments, the first reactor, the second reactor, and/or the third reactor may comprise a volume of working fluid of at least about a 1 gallon, at least about a 2 gallon, at least about a 3 gallon, at least about a 4 gallon, at least about a 5 gallon, at least about a 6 gallon, at least about a 7 gallon, at least about a 8 gallon, at least about a 9 gallon, at least about a 10 gallon, at least about a 11 gallon, at least about a 12 gallon, at least about a 13 gallon, at least about a 14 gallon, at least about a 15 gallon, at least about a 16 gallon, at least about a 17 gallon, at least about a 18 gallon, at least about a 19 gallon, at least about a 20 gallon, at least about a 25 gallon, at least about a 30 gallon, at least about a 40 gallon, or at least about a 50 gallons. In some embodiments, the first reactor, the second reactor, and/or the third reactor may comprise a volume of working fluid of at least about 50, 100, 200, 300, 400, 500, 600, 700, 800, 900, 1000, 1250, 1500, 2000, or greater than about 2000 gallons. In some embodiments, the first reactor, the second reactor, and/or the third reactor may comprise a volume of working fluid of at most about 2000, 1500, 1250, 1000, 900, 800, 700, 600, 500, 400, 300, 200, 100, 50, or less than about 50 gallons.

In some embodiments, the total volume of working fluid within all of the reactors and clarifier may be maintained at 17.5 gallons or 60 gallons. In some embodiments, the total volume of working fluid within all of the reactors and clarifier may be maintained at a volume of at least about 1 gallon, 5 gallons, 10 gallons, 15 gallons, 20 gallons, 25 gallons, 50 gallons, 75 gallons, 100 gallons, 150 gallons, 200 gallons, 250 gallons, 500 gallons, 750 gallons, 1000 gallons, 2500 gallons, 5000 gallons, 7500 gallons, 10000 gallons, 12500 gallons, 15000 gallons, or greater than about 15000 gallons. In some embodiments, the total volume of working fluid within all of the reactors and clarifier may be maintained at a volume of at most about 15000 gallons, 12500 gallons, 10000 gallons, 7500 gallons, 5000 gallons, 2500 gallons, 1000 gallons, 750 gallons, 500 gallons, 250 gallons, 200 gallons, 150 gallons, 100 gallons, 75 gallons, 50 gallons, 25 gallons, 20 gallons, 15 gallons, 10 gallons, 5 gallons, 1 gallon, or less than about 1 gallon.

In some embodiments, the hydraulic rate of flow through the bioreactor system may be 4.6 ml/min to maintain a retention time of 10 days and can be varied accordingly for a retention time from 7 to 21 days depending on production volume requirements. In some embodiments, the hydraulic rate of flow through the bioreactor system may be a rate of 15 ml/min or 11 ml/min. In some embodiments, the hydraulic rate of flow through the bioreactor system may be a rate of at least about 1 ml/min, 2 ml/min, 3 ml/min, 4 ml/min, 4.5 ml/min, 5 ml/min, 5.5 ml/min, 6 ml/min, 6.5 ml/min, 7 ml/min, 7.5 ml/min, 8 ml/min, 9 ml/min, 10 ml/min, 11 ml/min, 12 ml/min, 13 ml/min, 14 ml/min, 14.5 ml/min, 15 ml/min, 15.5 ml/min, 16 ml/min, 16.5 ml/min, 17 ml/min, 17.5 ml/min, 20 ml/min, 22.5 ml/min, or 25 ml/min. In some embodiments, the hydraulic rate of flow through the bioreactor system may be a rate of at least about 0.1 gallon/min, 0.2 gallon/min, 0.3 gallon/min, 0.4 gallon/min, 0.5 gallon/min, 1.0 gallon/min, 1.5 gallon/min, 2.0 gallon/min, 2.5 gallon/min, 3.0 gallon/min, 3.5 gallon/min, 4.0 gallon/min, 4.5 gallon/min, 5.0 gallon/min, or greater than about 5.0 gallon/min. In some embodiments, the hydraulic rate of flow through the bioreactor system may be a rate of at most about 5.0 gallon/min, 4.5 gallon/min, 4.0 gallon/min, 3.5 gallon/min, 3.0 gallon/min, 2.5 gallon/min, 2.0 gallon/min, 1.5 gallon/min, 1.0 gallon/min, 0.5 gallon/min, 0.4 gallon/min, 0.3 gallon/min, 0.2 gallon/min, 0.1 gallon/min, or less than about 0.1 gallon/min.

A retention time may comprise a time a population of an inoculated microbial strain spends in a bioreactor system or a time a population of the microbial strain spends following transfer into a first container and until collection from the bioreactor system. A longer retention time may be advantageous for growth or enrichment of a population of the microbial strain of the bioreactor system. A shorter retention time may be advantageous for growth or enrichment of a population of the microbial strain of the bioreactor system. In some embodiments, the retention time of the bioreactor system may be 10 days.

In some embodiment, the bioreactor system may produce 1.75 gallons of base product flowing from the clarifiers per day. In some embodiment, the bioreactor system may produce 4.29 gallons of base product flowing from the clarifiers per day. In some embodiment, the bioreactor system may produce 6 gallons of base product flowing from the clarifiers per day. In some embodiments, the digestion system may produce at least about 0.5, 0.75, 1.0, 1.25, 1.5, 1.75, 2.0, 2.25, 2.5, 2.75, 3.0, 3.5, 4.0, 4.5, 5.0, 6.0, 7.0, 8.0, 9.0, 10.0, 12.0, 15.0, or greater than about 15.0 gallons of base product flowing from the clarifiers per day. In some embodiments, the digestion system may produce at most about 15.0 12.0, 10.0, 9.0, 8.0, 7.0, 6.0, 5.0, 4.5, 4.0, 3.5, 3.0, 2.75, 2.5, 2.25, 2.0, 1.75, 1.5, 1.25, 1.0, 0.75, 0.5, or less than about 0.5 gallons of base product flowing from the clarifiers per day.

In some embodiments, the produced base products from the bioreactor system can have a pH of 7.39-8.88. In some embodiments, the produced base products from the digestion system can have a pH of at least about 4, 4.5, 5, 5.5, 6, 6.5, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10. In some embodiments, the produced base products from the digestion system can have a pH of at most about 10, 9.9, 9.8, 9.7, 9.6, 9.5, 9.4, 9.3, 9.2, 9.1, 9, 8.9, 8.8, 8.7, 8.6, 8.5, 8.4, 8.3, 8.2, 8.1, 8, 7.9, 7.8, 7.7, 7.6, 7.5, 7.4, 7.3, 7.2, 7.1, 7, 6.5, 6, 5.5, 5, 4.5, 4, or less than 4.

In some embodiments, the produced base products from the bioreactor system can have a total nitrogen (N) content of 0.02-0.07%. In some embodiments, the produced base products from the digestion system can have a total nitrogen (N) content in a working fluid of the digestion system of at least about 0.005%, 0.01%, 0.0125%, 0.015%, 0.0175%, 0.02%, 0.0225%, 0.025%, 0.0275%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.2%, 0.3%, 0.4%, 0.5%, 0.75%, 1.0%, 1.5%, 2.0%, or greater than about 2.0% relative to a total composition of the working fluid.

In some embodiments, the produced base products from the bioreactor system can have a total phosphorous (P) content of 0.00001%. In some embodiments, the produced base products from the digestion system can have a total phosphorous (P) content in a working fluid of the digestion system of at least about 0.00001%, 0.00005%, 0.0001%, 0.0005%, 0.001%, 0.0025%, 0.005%, 0.0075%, 0.01%, 0.025%, 0.05%, 0.075%, 0.1%, 0.15%, or 0.2%. In some embodiments, the produced base products from the digestion system can have a total phosphorous (P) content in a working fluid of the digestion system of at most about 0.2% 0.15%, 0.1%, 0.075%, 0.05%, 0.025%, 0.01%, 0.0075%, 0.005%, 0.0025%, 0.001%, 0.0005%, 0.0001%, 0.00005%, 0.00001%, or less than about 0.00001%.

### III. Biostimulant Compositions

Biostimulant compositions produced by a digestion process as described herein may be used as—is or may be further processed before being used. For example, the outflow from the bioreactor system, referred to herein as “base product,” may be concentrated, sterilized, filtered, pasteurized, dehydrated, or otherwise processed before being used, or any combination of these. In some embodiments, the base product may be concentrated by at least about 2×, at least about 3×, at least about 4×, at least about 5×, at least about 6×, at least about 7×, at least about 8×, at least about 9×, at least about 10×, or greater than about 10×. In some embodiments, the base product may be concentrated by at most about 10×, at most about 9×, at most about 8×, at most about 7×, at most about 6×, at most about 5×, at most about 4×, at most about 3×, at most about 2×, or less than 2×. In some embodiments, the base product may be filter sterilized to remove any bacteria or other microbes in the composition.

In an aspect, provided herein is a method comprising transferring a feedstock (e.g., aqueous organic feedstock) into a first container. An inoculum of a microbe may be transferred into a first container. A feedstock (e.g., an aqueous organic feedstock) may be transferred into a first container. A feedstock (e.g., an aqueous organic feedstock) and an inoculum of a microbe may be transferred into a first container. The first container may comprise a volume of a first working fluid. The feedstock (e.g., aqueous organic feedstock) may comprise a microbial consortium. The feedstock (e.g., aqueous organic feedstock) may comprise digestion products produced by digestion of an organic material. The feedstock (e.g., aqueous organic feedstock) may comprise a microbial consortium and one or more digestion products produced by digestion of an organic material. In some cases, the feedstock may comprise at least about 1, 2, 3, 4, 5, 6, 7, 8, 9, 10, or greater than about 10 digestion products. In some cases, the feedstock may comprise at most about 10, 9, 8, 7, 6, 5, 4, 3, 2, or less than 2 digestion products. The organic material may be digested by one or more microbes in the microbial consortium. The organic material may be digested by a population of microbes of the inoculum of a microbe (e.g., a zinc solubilizing target isolate, a phosphate solubilizing target isolate). The digestion products described herein may comprise one or more sugars (e.g., monosaccharides, disaccharides, oligosaccharides, polysaccharides, or any combination thereof). In some embodiments, the digestion products described herein may comprise one or more sugars (e.g., xylose, mannose, glucose, or any combination thereof), one or more metabolites generated by microbes of the working fluid, one or more fatty acids, one or more dead microorganisms, one or more fragments of dead microorganisms, one or more microorganism fermentation products, one or more enzymes, one or more biological plant growth modulators, one or more organic acids, one or more chelators, or any combination thereof. The method may further comprise incubating the inoculum of a microbe under conditions that selectively promotes growth of the microbes and increases the population of the microbes. The method may further comprise incubating the inoculum of a microbe under conditions that selectively promotes growth of at least a portion of microbes in the microbial consortium. The terms “microbial digestion” and “digestion” can be used interchangeably.

In some embodiments, the digestion is anaerobic digestion. In some embodiments, the digestion is aerobic digestion. In some embodiments, the digestion is microaerobic digestion. In some embodiments, the digestion is aerobic digestion, microaerobic digestion, anaerobic digestion, or some combination thereof. Without wishing to be bound by theory, it is believed that during the digestion process, microbes digest the biomolecules and other nutrients present in the manure, yeast, kelp, and/or produce digestion products that comprise compounds that promote plant growth and soil health. In some embodiments of a digestion process, the feedstock (e.g., organic feedstock) may be mixed with water to make a feedstock (e.g., organic feedstock) for an anaerobic bioreactor system. The anaerobic bioreactor system may comprise a mixing tank in which the feedstock (e.g., organic feedstock) is mixed to make a fluid feed mixture or working fluid. In some embodiments, the fluid feed mixture may comprise manure, water, and Saccharomyces cerevisiae yeast. In some embodiments, anaerobic digestion comprises a process by which bacteria break down organic biomaterials in the absence of oxygen. The biostimulant may also contain microbes that contribute to the plant-beneficial properties of the biostimulant product. The microbes in the biostimulant product may be derived from the microbial population present in the feedstock (e.g., organic feedstock). The reactors of the bioreactor system may comprise mixers that agitate the working fluid and aerate the working fluid. The mixers may be configured to increase aerobic digestion and the increased aeration may facilitate enrichment of microbial communities of a target functionality (e.g., phosphate-solubilizing microbes, zinc-solubilizing microbes).

A biostimulant may enhance water uptake and/or nutrient utilization in a plant and improves soil quality. A biostimulant and/or application of a biostimulant may enhance a yield of a crop. A biostimulant can comprise a microbe and/or microorganism described herein. A biostimulant may be a product of a bioreactor system as described herein. The inoculum of a microbe described herein may comprise a plant growth promotion property for a biostimulant product. The inoculum of a microbe described herein may be added to a biostimulant comprising plant extracts, protein hydrolysates, chemical biostimulants, humic or fulvic acids, seaweed extracts, liquid manure, other beneficial bacteria and/or fungi (e.g., Bacillus or Rhizobium), or any combination thereof. The biostimulant composition can be added to a formulation (e.g., formulation containing fertilizer).

The biostimulant composition may comprise a population of zinc-solubilizing microbes enriched in the bioreactor system. In some embodiments, the biostimulant composition comprises a population of the inoculum of the zinc-solubilizing microbe, at least a portion of zinc-solubilizing microbes of the microbial consortium, zinc-solubilizing metabolites generated in the bioreactor system, or any combination thereof. In some embodiments, the metabolites generated in the bioreactor system also promote plant growth in ways described herein and promote micronutrient uptake. The zinc-solubilizers enriched in the bioreactor system described herein may be added to plant extracts, protein hydrolysates, humic acids, and/or chemical biostimulants. In some embodiments, the present disclosure provides a biostimulant composition comprising chemical species and/or microbes that promote plant growth, including increasing a plant's or crop's zinc use efficiency. In some embodiments, the biostimulant composition can be added to formulation (e.g., formulation containing fertilizer). Biostimulant compositions described herein may comprise dead microorganisms, sporulated microorganisms, fragments of dead microorganisms, viable microorganisms, microorganism digestion products, microbial metabolites and secondary metabolites, enzymes, biological plant growth modulators, organic acids, chelators, or any combination thereof.

The biostimulant composition may comprise a population of phosphate-solubilizing microbes enriched in the bioreactor system. In some embodiments, the biostimulant composition comprises a population of the inoculum of the phosphate-solubilizing microbe, at least a portion of phosphate-solubilizing microbes of the microbial consortium, phosphate-solubilizing metabolites generated in the bioreactor system, or any combination thereof. The phosphate-solubilizers enriched in the bioreactor system described herein may be added to plant extracts, protein hydrolysates, chemical biostimulants, humic or fulvic acids, seaweed extracts, liquid manure, other beneficial bacteria and/or fungi (e.g., Bacillus or Rhizobium), or any combination thereof. In some embodiments, the present disclosure provides a biostimulant composition comprising chemical species and/or microbes that promote plant growth, including increasing a plant's or crop's phosphate use efficiency. Biostimulant compositions described herein may comprise dead microorganisms, sporulated microorganisms, fragments of dead microorganisms, viable microorganisms, microorganism digestion products, microbial metabolites and secondary metabolites, enzymes, biological plant growth modulators, organic acids, chelators, or any combination thereof.

The biostimulant composition may comprise a population of nitrogen use efficiency-promoting microbes enriched in the bioreactor system. In some embodiments, the biostimulant composition comprises a population of the inoculum of the nitrogen use efficiency-promoting microbe, at least a portion of nitrogen use efficiency-promoting microbes of the microbial consortium, nitrogen use efficiency-promoting metabolites generated in the bioreactor system, or any combination thereof. The nitrogen use efficiency-promoting microbes enriched in the bioreactor system described herein may be added to plant extracts, protein hydrolysates, and/or chemical biostimulants. In some embodiments, the present disclosure provides a biostimulant composition comprising chemical species and/or microbes that promote plant growth, including increasing a plant's or crop's nitrogen use efficiency. Biostimulant compositions described herein may comprise dead microorganisms, sporulated microorganisms, fragments of dead microorganisms, viable microorganisms, microorganism digestion products, microbial metabolites and secondary metabolites, enzymes, biological plant growth modulators, organic acids, chelators, or any combination thereof.

In some embodiments, biostimulant compositions described herein may be characterized by mass spectrometry or NMR spectroscopy. In some embodiments, the biostimulant composition has an associated LC-MS chromatogram. In some embodiments, the biostimulant composition has an associated GC-MS chromatogram. In some embodiments, the biostimulant composition has an associated 1H-NMR spectrum. In some embodiments, the biostimulant composition has an associated 13C-NMR spectrum.

In some embodiments, biostimulant compositions can comprise one or more microbes. The biostimulant composition can comprise at least about 1, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 50, or greater than about 50 microbes. The biostimulant composition can comprise at most about 50, at most about 40, at most about 305, at most about 30, at most about 25, at most about 20, at most about 15, at most about 10, at most about 9, at most about 8, at most about 7, at most about 6, at most about 5, at most about 4, at most about 3, at most about 2, or less than about 2 microbes. In some embodiments, biostimulant compositions can comprise viable microbes. In some embodiments, the microbes comprise bacteria that may be derived from the bacterial population present in manure, rock phosphate, or other digestion substrates. In some embodiments, the microbes comprise bacteria that may be added to a bioreactor system as isolated microbial strains. In some embodiments, the bacteria may comprise one or more bacteria of the genera: Kosakonia, Klebsiella, Rahnella, Kluyvera, Enterobacter, Achromobacter, Microbacterium, Gluconobacter, Methylobacterium, Pseudomonas, Pantoea, Azospirillum, Azocarus, Herbaspirillum, Burkholderia, Cyanobacteria, Bacillus, or Paenibacillus. In some embodiments, the bacteria comprise one or more bacteria of the species Bacillus safensis, Bacillus amyloliquefaciens, Bacillus licheniformis, Paenibacillus borealis, Paenibacillus sonchi, Bacillus megaterium, Pseudomonas spp., Agrobacterium spp., Bacillus circulans, Burkholderia lata, Lysinibacillus sp., Burkholderia latens, Bacillus cereus, B. aryabhattai, Pseudomonas taiwenensis, Acinetobacter sp., Paenibacillus sonchi, Chitinophaga spp., Gluconobacter spp., Klebsiella spp., Serratia spp., or Thiobacillus spp. In some embodiments, the bacteria comprise one or more of microbial strains MS4666 (ATCC Accession No. PTA-127681), MS4689 (ATCC Accession No. PTA-127683), MS4687 (ATCC Accession No. PTA-127682), MS2839 (ATCC Accession No. PTA 127657), MS1835 (ATCC Accession No. PTA-127656), MS3907 (ATCC Accession No. PTA-127654), MS3900 (ATCC Accession No. PTA-127653), MS4921 (ATCC Accession No. PTA-127655), or MS2748 (ATCC Accession No. PTA-127652). In some embodiments, any one or any combination of these bacterial species comprises at least about 0.000001%, at least about 0.00001%, at least about 0.00005%, at least about 0.0001%, at least about 0.0005%, at least about 0.001%, at least about 0.0015%, at least about 0.002%, at least about 0.003%, at least about 0.004%, at least about 0.005%, at least about 0.01%, at least about 0.05%, at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, or more than about 20% of the total bacterial species present in the biostimulant, for example, as determined by metagenomic sequencing or semi-quantitative PCR sequencing. In some embodiments, any one of these bacterial species comprises at most about 20%, at most about 15%, at most about 14%, at most about 13%, at most about 12%, at most about 11%, at most about 10%, at most about 9%, at most about 8%, at most about 7%, at most about 6%, at most about 5%, at most about 4%, at most about 3%, at most about 2%, at most about 1%, at most about 0.5%, at most about 0.1%, at most about 0.05%, at most about 0.01%, at most about 0.005%, at most about 0.004%, at most about 0.003%, at most about 0.002%, at most about 0.0015%, at most about 0.0005%, at most about 0.0001%, at most about 0.00005%, at most about 0.00001%, at most about 0.000001%, or less than about 0.000001% of the bacterial species present in the biostimulant, for example as determined by metagenomic sequencing or semi-quantitative PCR sequencing.

In some embodiments, the biostimulant can be filter sterilized. The biostimulant may comprise microbes (e.g., viable microbes). The biostimulant may not comprise microbes (e.g., viable microbes). In some embodiments, the dry weight of the microbial biomass can be at most about 0.01%, 0.005%, 0.001%, 0.0005%, 0.0001%, or less than about 0.0001% in relation to the total dry weight of the biostimulant composition. In some embodiments, the biostimulant compositions may comprise metabolites. The metabolites may be derived from the microbial communities of the bioreactor system described herein. Transferring of working fluid into a container of the bioreactor system may generate metabolites. Metabolites may be generated by microbial metabolism. Metabolites may be generated by enzymes catalyzing biochemical reactions of the organic substrates of the aqueous organic feedstock in a working fluid of a digestion system as described herein. The metabolites may be produced with introduction of the microbial strain (e.g., isolate or target isolate). The metabolites may be produced without introduction of the microbial strain (e.g., isolate or target isolate).

The metabolites may comprise one or more plant growth promoting properties. The plant growth promoting property can be a property as described herein. In some embodiments, metabolites produced by the bioreactor systems described herein may enhance nutrient availability and/or nutrient use efficiency.

In some embodiments, the biostimulant comprises about 1×10⁸ CFU/ml of bacteria. In some embodiments, the biostimulant comprises at least about 100 CFU/ml, 500 CFU/ml, 1×10³ CFU/ml, 1×10⁴ CFU/ml, 1×10⁵ CFU/ml, 1×10⁶ CFU/ml, 5×10⁶ CFU/ml, 1×10⁷ CFU/ml, 5×10⁷ CFU/ml, 1×10⁸ CFU/ml, 5×10⁸ CFU/ml, 1×10⁹ CFU/ml, 5×10⁹ CFU/ml, 1×10¹⁰ CFU/ml, 1×10¹¹ CFU/ml 1×10¹² CFU/ml 1×10¹³ CFU/ml 1×10¹⁴ CFU/ml, or 1×10¹⁵ CFU/ml of bacteria. In some embodiments, the biostimulant comprises at most about 100 CFU/ml, 500 CFU/ml, 1×10³ CFU/ml, 1×10⁴ CFU/ml, 1×10⁵ CFU/ml, 1×10⁶ CFU/ml, 5×10⁶ CFU/ml, 1×10⁷ CFU/ml, 5×10⁷ CFU/ml, 1×10⁸ CFU/ml, 5×10⁸ CFU/ml, 1×10⁹ CFU/ml, 5×10⁹ CFU/ml, 1×10¹⁰ CFU/ml, 1×10¹¹ CFU/ml 1×10¹² CFU/ml 1×10¹³ CFU/ml 1×10¹⁴ CFU/ml, or 1×10¹⁵ CFU/ml of bacteria.

In some embodiments, the biostimulant has a pH of from 7 to 8. In some embodiments, the biostimulant has a pH of at least about 4, 4.5, 5, 5.5, 6, 6.5, 7, 7.5, 8, 8.5, 9, 9.5, 10, 10.5, 11, or greater than 11. In some embodiments, the biostimulant has a pH of at most about 11, 10.5, 10, 9.5, 9, 8.5, 8, 7.5, 7, 6.5, 6, 5.5, 5, 4.5, 4, or less than 4. In some embodiments, the density of the biostimulant is about 0.997 to 0.999 g/cm³ or is about 0.998 g/cm³.

In some embodiments, the density of the biostimulant is at least about 0.5 g/mL, 0.6 g/mL, 0.7 g/mL, 0.8 g/mL, 0.9 g/mL, 1.0 g/mL, 1.1 g/mL, 1.2 g/mL, 1.3 g/mL, 1.4 g/mL, 1.5 g/mL, or more. In some embodiments, the density of the biostimulant is at most about 0.5 g/mL, 0.6 g/mL, 0.7 g/mL, 0.8 g/mL, 0.9 g/mL, 1.0 g/mL, 1.1 g/mL, 1.2 g/mL, 1.3 g/mL, 1.4 g/mL, or 1.5 g/mL. In some embodiments, the biostimulant has a solids content of 0.01 to 2%.

In some embodiments, the biostimulant has a solids content of at least about 0.001%, 0.01%, 0.05%, 0.1%, 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 3%, 4%, 5% w/w, or greater than about 5% w/w. In some embodiments, the biostimulant has a solids content of at most about 0.001%, 0.01%, 0.05%, 0.1%, 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 3%, 4%, or 5% w/w.

In some embodiments, the chemical oxygen demand (COD) of the biostimulant is from 100 to 250 mg/L. In some embodiments, the COD is at least about 10, 25, 50, 75, 100, 125, 150, 175, 200, 225, 250, 275, 300, 400, or 500 mg/L. In some embodiments, the COD is at most about 10, 25, 50, 75, 100, 125, 150, 175, 200, 225, 250, 275, 300, 400, or 500 mg/L. COD values may vary with the concentration rate of the biostimulant, and may increase as concentration increases.

In some embodiments, the biostimulant has a total nitrogen content of about 0.0002% w/w or between about 0.0001% and 0.0003% w/w. In some embodiments, the biostimulant has a total nitrogen content of at least about 0.00001%, 0.00005%, 0.0001%, 0.0002%, 0.0003%, 0.0004%, 0.0005%, 0.001%, 0.01%, or greater than about 0.01% w/w. In some embodiments, the biostimulant has a total nitrogen content of at most about 0.01% 0.001%, 0.0005%, 0.0004%, 0.0003%, 0.0002%, 0.0001%, 0.00005%, 0.00001%, or less than about 0.00001% w/w. In some embodiments, the biostimulant has a total phosphorous content of about 0.00001% w/w or between about 0.000005% and 0.000015% w/w. In some embodiments, the biostimulant has a total phosphorus content of at least about 0.00001%, 0.00002%, 0.00003%, 0.00004%, 0.00005%, 0.00006%, 0.00007%, 0.00008%, 0.00009%, 0.0001%, 0.0002%, 0.0003%, 0.0004%, 0.0005%, 0.001%, 0.01%, or greater than about 0.01%. In some embodiments, the biostimulant has a total phosphorus content of at most about 0.01%, 0.001%, 0.0005%, 0.0004%, 0.0003%, 0.0002%, 0.0001%, 0.00009%, 0.00008%, 0.00007%, 0.00006%, 0.00005%, 0.00004%, 0.00003%, 0.00002%, 0.00001%, or less than about 0.00001%. In some embodiments, the biostimulant has a total potassium content of about 0.0001% w/w or between about 0.00005% and 0.00015% w/w. In some embodiments, the biostimulant has a total potassium content of at least about 0.00001, 0.00005%, 0.00006%, 0.00007%, 0.00008%, 0.00009%, 0.0001%, 0.0002%, 0.0003%, 0.0004%, 0.0005%, 0.001%, or 0.01%. In some embodiments, the biostimulant has a total potassium content of at most about 0.01%, 0.001%, 0.0005%, 0.0004%, 0.0003%, 0.0002%, 0.0001%, 0.00009%, 0.00008%, 0.00007%, 0.00006%, 0.00005%, 0.00004%, 0.00003%, 0.00002%, 0.00001%, or less than about 0.00001%.

### IV. Methods of Making a Biostimulant Composition

In some embodiments, a bioreactor system that is used in embodiments of methods of making a biostimulant have previously been inoculated with a microbial strain with desired plant growth promoting characteristics (a “target strain”) such as, for example, zinc-solubilizing properties. In some embodiments, a bioreactor system that is used in embodiments of methods of making a biostimulant have previously been inoculated with a microbial strain with desired plant growth promoting characteristics (a “target strain”) such as, for example, phosphate solubilizing properties. In some embodiments, a bioreactor system that is used in embodiments of methods of making a biostimulant have previously been inoculated with a microbial strain with desired plant growth promoting characteristics (a “target strain”) such as, for example, nitrogen use efficiency-promoting properties. The inoculation may have been performed at least about 1 day, 2 days, 3 days, 4 days, 5 days, 10 days, 15 days, 30 days, 60 days, 90 days, 120 days, or greater than about 120 days previous to the beginning of a duration of time in which embodiments of the method are performed. The inoculation may have been performed at most about 120 days, 90 days, 60, 30, 15, 10, 5, 4, 3, 2, 1, or less than about 1 day previous to the beginning of a duration of time in which embodiments of the method are performed. The inoculation may have been performed at least about 5 months, 6 months, 7 months, 8 months, 9 months, 10 months, 11 months, or greater than about 11 months previous to the beginning of a duration of time in which embodiments of the method are performed. The inoculation may have been performed at most about 11 months, 10 months, 9 months, 8 months, 7 months, 6 months, 5 months, or less than about 5 months previous to the beginning of a duration of time in which embodiments of the method are performed. The inoculation may have been performed at least about 1 year, 2 years, 3 years, 4 years, 5 years, or greater than about 5 years previous to the beginning of a duration of time in which embodiments of the method are performed. The inoculation may have been performed at most about 5 years, 4 years, 3 years, 2 years, 1 years, or less than about 1 year previous to the beginning of a duration of time in which embodiments of the method are performed. In some embodiments, an inoculum establishes an initial population in one or more containers of a bioreactor system of at least about 1×10² CFU/ml, at least about 1×10³ CFU/ml, at least 1×10⁴ CFU/ml, at least about 1×10⁵ CFU/ml, at least about 1×10⁶ CFU/ml, at least about 1×10⁷ CFU/ml, or greater than about 1×10⁷ CFU/ml. In some embodiments, an inoculum establishes an initial population in one or more containers of a bioreactor system of at most 1×10⁷ CFU/ml, 1×10⁶ CFU/ml, 1×10⁵ CFU/ml, 1×10⁴ CFU/ml, 1×10³ CFU/ml, 1×10² CFU/ml, or less than about 1×10² CFU/ml. Without being bound by theory, an inoculation of a target strain may help establish a population of the target strain and/or of other microbes having similar plant growth promoting properties as the target strain that is maintained throughout a duration of time of operating the bioreactor system. In some embodiments, an inoculum of the target strain can be added to the system only once or no more than once every 1, 2, 3, 4, 5, or 10 months. In some embodiments, the inoculum of the strain comprises at least about 1×10⁴, 1×10⁵, 1×10⁶, 1×10⁷, 1×10⁸, 1×10⁹, 1×10¹⁰, or 1×10¹¹ total CFU of the target strain. In some embodiments, the inoculum of the strain comprises at most about 1×10⁴, 1×10⁵, 1×10⁶, 1×10⁷, 1×10⁸, 1×10⁹, 1×10¹⁰, or 1×10¹¹ total CFU of the target strain.

In some embodiments, the inoculum may comprise at least about 1, at least about 2, at least about 3, at least about 4, at least about 5, at least about 10, at least about 15, at least about 20, at least about 25, at least about 50, or greater than about 50 different microbial species. In some embodiments, the inoculum comprises only a single target strain and no other microbes. In some embodiments, the inoculum comprises at most about 50, at most about 25, at most about 20, at most about 15, at most about 10, at most about 5, at most about 4, at most about 3, at most about 2, at most about 1, or less than about 1 microbial strains (e.g., target microbial strains). In some embodiments, the inoculum comprises only a single target strain but is mixed with other microbes. In some embodiments, the target strain is chosen to be added to the bioreactor based on its plant growth promoting properties. In some embodiments, the inoculum is not a part of or mixed with an organic feed material such as, for example, manure, food waste, wastewater, or sewage.

In some embodiments, operating a bioreactor system comprises continuously providing a feedstock inflow into the system, continuously transferring working fluid from each container in the system to a subsequent downstream container in the system, and continuously collecting a product stream from the system. In some embodiments, the flow into and/or out of a container may be performed periodically, for example a volume of feed or working fluid may be transferred once every at least 1, 5, 10, 30, or 60 seconds. In some embodiments, operating a bioreactor system does not comprise incubating a fluid in a container without inflow or outflow for more than about 1, 5, 10, 15, or 30 minutes, or 1, 5, or 10 hours. In some embodiments, operating the bioreactor system comprises flowing a particular volume of fluid both into and out of the bioreactor system in a particular amount of time. For example, operating the bioreactor system may comprise flowing a volume of fluid that is equal to a fluid capacity of a container of the bioreactor system into and out of the system in 1 hours, 2 hours, 3 hours, 4 hours, 5 hours, 10 hours, 12 hours, 24 hours, 48 hours, 3 days, 4 days, 5 days, 6 days, 7 days, 8 days, 9 days, 10 days, 11 days, 12 days, 13v, 14 days, 15 days, 16 days, 17 days, 18 days, 19 days, 20 days, or greater than about 20 days. For example, for a bioreactor system that has a container with a 500 gallon fluid capacity, operating the bioreactor system may comprise flowing 500 gallons into the system and flowing 500 gallons out of the system in 12 hours. Operating a bioreactor system may also comprise flowing fluid through the system at a certain rate given in units of volume over time. For example, operating a bioreactor system may comprise flowing fluid through the system at a rate of at least about 0.001, 0.005, 0.01, 0.05, 0.1, 0.5, 1.0, 1.5, 2.0, 2.5, 3.0, 3.5, 4.0, 4.5, 5.0, 10.0, 20.0, 30.0, 40.0, 50.0, or greater than about 50.0 gallons per minute. Operating a bioreactor system may comprise flowing fluid through the system at a rate of at most about 50.0, 40.0, 30.0, 20.0, 10.0, 5.0, 4.5, 4.0, 3.5, 3.0, 2.5, 2.0, 1.5, 1.0, 0.5, 0.1, 0.05, 0.01, 0.005, 0.001, or less than about 0.001 gallons per minute. Operating a bioreactor system may also comprise flowing fluid through the system at a certain rate that results in a particular hydraulic retention time. For example, operating a bioreactor system may comprise flowing fluid through the system at a rate that results in a hydraulic retention time of at least about 5 hours, 10 hours, 12 hours, 1 days, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, or greater than about 25 days.

In some embodiments, a population of a target strain in a container is maintained at a certain level or within a certain range during operation of the bioreactor system for a given duration of time despite the target strain not being present in any inflow into the bioreactor system during the duration of time. In some embodiments, a population of a target strain in a container may be maintained at a certain level or within a certain range during operation of the bioreactor system for a given duration of time despite the target strain being present only in at most relatively low levels in any inflow into the bioreactor system during the duration of time. For example, the level of the target strain flowing into the system may be at least 1000 times lower than the population of the target strain in one or more containers of the bioreactor system. For example, in some embodiments, the population of the target strain is maintained at a level of at least 1×10⁶ CFU/ml or at least 80% of that level, while the total inflow into the bioreactor system comprises no more than 1×10² CFU/ml of the target strain. Thus, in some embodiments, the conditions in the bioreactor are such that the growth rate of the target isolate in one or more containers of the bioreactor system is high enough that the continuous flow of fluid through the system does not substantially deplete the population of the target isolate in the one or more containers. In some embodiments the population of the target isolate is maintained at least about 1×10⁴, 1×10⁵, 1×10⁶, 1×10⁷, or 1×10⁸ CFU/ml, or at least 80% of that level for a period of time of operating the bioreactor system, while the total input into the system does not comprise the target strain or does not comprise more than 1, 10, 100, 500, 1000, 1500, 2000, or 5000 CFU/ml of the target strain. In some embodiments, for a duration of time of operating the bioreactor system, the population of the target isolate in one or more containers may be maintained at least about 70%, 80%, 85%, 90%, 95%, 96%, 98%, 99%, or 100% of the population of the target isolate that was present at the beginning of the duration of time. In some embodiments, the population of the target strain is maintained at a level at least about 100, 500, 1000, 5000, 10000, 15000, or 20000 times higher than the population of the target strain in all inputs into the bioreactor system for a duration of time of operating the bioreactor system. In some embodiments, the duration of time of operating the bioreactor system during which the particular population is maintained at a certain level or within a certain range is at least 1, 5, 10, 15, 30, 45, 60, 75, or 90 days, at least 6, 9, 12, 15, 18, or 24 months, or at least 3, 4, 5, 6, 7, 8, 9, or 10 years.

In some embodiments, one or more containers comprise a population of one or more other microbes having a desirable plant growth promoting property (such as, for example, zinc solubilizing properties, phosphate solubilizing properties, nitrogen use efficiency promoting properties) in addition to the population of the target isolate. In some embodiments, the population of these one or more other desirable microbes (e.g., those one or more microbes with the plant growth promoting property) can also be maintained at a certain level or within a certain range during a period of time of operating the bioreactor system, despite being present at much lower levels in the inputs into the bioreactor system. The other desirable microbes (e.g., those one or more microbes with the plant growth promoting property) may be from an input microbial consortium to the bioreactor system. For example, the level of the other desirable microbes (e.g., those one or more microbes with the plant growth promoting property) flowing into the system may be at least 1000 times lower than the population of the other desirable microbes in one or more containers of the bioreactor system. For example, in some embodiments, the population of the other desirable microbes (e.g., those one or more microbes with the plant growth promoting property) may be maintained at a level of at least 1×10⁶ CFU/ml or at least 80% of that level, while the total inflow into the bioreactor system comprises no more than 1×10² CFU/ml of the other desirable microbes. Thus, in some embodiments, the conditions in the bioreactor are such that the growth rate of the other desirable microbes (e.g., those one or more microbes with the plant growth promoting property) in one or more containers of the bioreactor system is high enough that the continuous flow of fluid through the system does not deplete (e.g., fully deplete) the population of the other desirable microbes in the one or more containers. Thus, in some embodiments, the conditions in the bioreactor are such that the growth rate of the other desirable microbes (e.g., those one or more microbes with the plant growth promoting property) in one or more containers of the bioreactor system is high enough that the continuous flow of fluid through the system does not partially deplete (e.g., not fully deplete) the population of the other desirable microbes in the one or more containers.

In some embodiments the population of the other desirable microbes is maintained at least 1×10⁴, 1×10⁵, 1×10⁶, 1×10⁷, or 1×10⁸ CFU/ml, or at least 80% of that level for a period of time of operating the bioreactor system, while the total input into the system does not comprise the other desirable microbes or does not comprise more than 1, 10, 100, 500, 1000, 1500, 2000, or 5000 CFU/ml of the other desirable microbes. In some embodiments, for a duration of time of operating the bioreactor system, the population of the other desirable microbes in one or more containers is maintained at least 70%, 80%, 85%, 90%, 95%, 96%, 98%, 99%, or 100% of an initial population of the other desirable microbes present at the beginning of the duration of time. In some embodiments, the population of other desirable microbes is maintained at a level at least 100, 500, 1000, 5000, 10000, 15000, or 20000 times higher than the population of the other desirable microbes in all inputs into the bioreactor system for a duration of time of operating the bioreactor system. In some embodiments, the duration of time of operating the bioreactor system during which the particular population is maintained at a certain level or within a certain range is at least 1, 5, 10, 15, 30, 45, 60, 75, or 90 days, or at least 6, 9, 12, 15, 18, or 24 months.

### V. Biostimulant Formulations, and Methods of Use

The bacterial isolates described herein can be formulated as a biostimulant for plant application to enhance plant growth promotion properties. A biostimulant composition as described herein can comprise target bacterial isolates of the present disclosure mixed with other components in a tank or similar container. The formulations can be bottled or otherwise packaged, applied to a plant, a seed, a field, a crop, and/or a growth medium, and/or mixed with other components. When bottled or otherwise packaged, the end user can mix the formulation with other components prior to application. The biostimulant composition can be mixed with fertilizer by tank mixing, including splash mixing with minimal further mixing, or can be blended into the fertilizer.

In some embodiments, the microbial formulation and/or biostimulant described herein has zinc solubilizing properties. The zinc solubilizing microbes may enhance zinc uptake in plants. The zinc solubilizing microbes may enhance zinc bioavailability in soil. In some embodiments, the microbial formulation and/or biostimulant described herein has phosphate solubilizing properties. The phosphate solubilizing microbes may enhance phosphate uptake in plants. The phosphate solubilizing microbes may enhance phosphate bioavailability in soil. In some embodiments, the microbial formulation and/or biostimulant described herein may be used in methods of increasing nitrogen use efficiency of a plant or crop.

In some embodiments, the biostimulant may be applied to the plant, the seed, the field, the crop, or the growth medium before planting. In some embodiments, the biostimulant may be applied at planting or after planting. In some embodiments, the biostimulant may be applied before transplanting the plant. In some embodiments, the biostimulant is applied to the plant or growth medium after transplanting the plant. In some embodiments, the biostimulant may be applied to the plant or growth medium while the plant is being transplanted. In some embodiments, the biostimulant may be applied to a growth medium (e.g., soil) into which the plant is to be transplanted. In some embodiments, the biostimulant may be applied to a fertilizer or incorporated into a fertilizer, which is applied before planting, at planting or after planting. In some embodiments, the biostimulant may be applied as a foliar application (e.g., applied as a liquid fertilizer to the leaves). The biostimulant may be applied as a foliar application at various times after planting. For example, the biostimulant may be applied as a foliar application at least about 1 hour, 2 hours, 3 hours, 4 hours, 5 hours, 6 hours, 9 hours, 12 hours, 18 hours, 24 hours, 2 days, 3 days, 4 days, 5 days, 6 days, 7 days, 2 weeks, 3 weeks, 4 weeks, 2 months, 3 months, 4 months, 5 months, 6 months, 9 months, 12 months, 15 months, 18 months, 24 months, 3 years, 4 years, or 5 years after planting. The biostimulant may be applied as a foliar application at most about 5 years, 4 years, 3 years, 24 months, 18 months, 15 months, 12 months, 9 months, 6 months, 5 months, 4 months, 3 months, 2 months, 4 weeks, 3 weeks, 2 weeks, 7 days, 6 days, 5 days, 4 days, 3 days, 2 days, 24 hours, 18 hours, 12 hours, 9 hours, 6 hours, 5 hours, 4 hours, 3 hours, 2 hours, 1 hour, or less than 1 hour after planting.

In some embodiments, the plant treated with the biostimulant composition may be, for example, crops, vegetables, flowers, foliage plants, turf grasses, trees, shrubs, and the like. Non-limiting examples of crops comprise corn, rice, wheat, barley, rye, oat, sorghum, cotton, soybean, peanut, buckwheat, beet, rapeseed, sunflower, sugar cane, marijuana, and tobacco. Vegetables may comprise solanaceous vegetables (eggplant, tomato, pimento, pepper, or potato), cucurbitaceous vegetables (cucumber, pumpkin, zucchini, watermelon, melon, or squash), cruciferous vegetables (Japanese radish, white turnip, horseradish, kohlrabi, Chinese cabbage, cabbage, leaf mustard, broccoli, or cauliflower), asteraceous vegetables (burdock, crown daisy, artichoke, or lettuce), liliaceous vegetables (green onion, onion, garlic, or asparagus), ammiaceous vegetables (carrot, parsley, celery, or parsnip), chenopodiaceous vegetables (spinach or Swiss chard), lamiaceous vegetables (Perilla frutescens, mint, or basil), strawberry, sweet potato, Dioscorea japonica, Colocasia, or any combination thereof. Fruits may comprise pomaceous fruits (apple, pear, Japanese pear, Chinese quince, or quince), stone fleshy fruits (peach, plum, nectarine, Prunus mume, cherry fruit, apricot, or prune), citrus fruits (Citrus unshiu, orange, lemon, rime, or grapefruit), nuts (chestnuts, walnuts, hazelnuts, almond, pistachio, cashew nuts, or macadamia nuts), berries (blueberry, cranberry, blackberry, or raspberry), grape, kaki fruit, olive, Japanese plum, banana, coffee, date palm, coconuts, or any combination thereof. Trees may comprise fruit trees, tea, mulberry, flowering plant, roadside trees (ash, birch, dogwood, Eucalyptus, Ginkgo biloba, lilac, maple, Quercus, poplar, Judas tree, Liquidambar formosana, plane tree, Zelkova, Japanese arborvitae, fir wood, hemlock, juniper, Pinus, Picea, or Taxus cuspidate), or any combination thereof. The term “plant” or “plants” refers to both native and genetically engineered plants. In some embodiments, the biostimulant is applied to a seed of any of the plants as described herein.

In some embodiments, the microbial formulation described herein have nitrogen use efficiency-promoting properties. The nitrogen use efficiency-promoting microbes may enhance nitrogen fixation in plants. The nitrogen use efficiency-promoting microbes may enhance bioavailability of nitrogen in soil. The nitrogen use efficiency-promoting microbes may enhance plant growth in low nitrogen conditions (e.g., a soil that does not contain nitrogen or nitrogen levels below 10 ppm). Low nitrogen conditions in a soil may comprise conditions of less than about 80%, 70%, 60%, 50%, 40%, or 30% the recommended nitrogen level suitable for crop growth as specified by a soil test. For example, a nitrogen-poor growth medium may comprise nitrate at a concentration of less than about 1 ppm, 5, 10, 12, 15, 18, 20, 25, 30, 35, 40, 50, 75, 100 or less than 100 ppm. A nitrogen-poor growth medium may comprise nitrate at a concentration of at most about 100, 75, 50, 40, 35, 30, 25, 20, 18, 15, 12, 10, 5, 1, or less than 1 ppm. A nitrogen-poor growth medium may comprise nitrate at a concentration from about 1 ppm to about 50 ppm. A nitrogen-poor growth medium may comprise nitrate at a concentration from about 1 ppm to about 2 ppm, about 1 ppm to about 3 ppm, about 1 ppm to about 4 ppm, about 1 ppm to about 5 ppm, about 1 ppm to about 10 ppm, about 1 ppm to about 15 ppm, about 1 ppm to about 20 ppm, about 1 ppm to about 25 ppm, about 1 ppm to about 30 ppm, about 1 ppm to about 40 ppm, about 1 ppm to about 50 ppm, about 2 ppm to about 3 ppm, about 2 ppm to about 4 ppm, about 2 ppm to about 5 ppm, about 2 ppm to about 10 ppm, about 2 ppm to about 15 ppm, about 2 ppm to about 20 ppm, about 2 ppm to about 25 ppm, about 2 ppm to about 30 ppm, about 2 ppm to about 40 ppm, about 2 ppm to about 50 ppm, about 3 ppm to about 4 ppm, about 3 ppm to about 5 ppm, about 3 ppm to about 10 ppm, about 3 ppm to about 15 ppm, about 3 ppm to about 20 ppm, about 3 ppm to about 25 ppm, about 3 ppm to about 30 ppm, about 3 ppm to about 40 ppm, about 3 ppm to about 50 ppm, about 4 ppm to about 5 ppm, about 4 ppm to about 10 ppm, about 4 ppm to about 15 ppm, about 4 ppm to about 20 ppm, about 4 ppm to about 25 ppm, about 4 ppm to about 30 ppm, about 4 ppm to about 40 ppm, about 4 ppm to about 50 ppm, about 5 ppm to about 10 ppm, about 5 ppm to about 15 ppm, about 5 ppm to about 20 ppm, about 5 ppm to about 25 ppm, about 5 ppm to about 30 ppm, about 5 ppm to about 40 ppm, about 5 ppm to about 50 ppm, about 10 ppm to about 15 ppm, about 10 ppm to about 20 ppm, about 10 ppm to about 25 ppm, about 10 ppm to about 30 ppm, about 10 ppm to about 40 ppm, about 10 ppm to about 50 ppm, about 15 ppm to about 20 ppm, about 15 ppm to about 25 ppm, about 15 ppm to about 30 ppm, about 15 ppm to about 40 ppm, about 15 ppm to about 50 ppm, about 20 ppm to about 25 ppm, about 20 ppm to about 30 ppm, about 20 ppm to about 40 ppm, about 20 ppm to about 50 ppm, about 25 ppm to about 30 ppm, about 25 ppm to about 40 ppm, about 25 ppm to about 50 ppm, about 30 ppm to about 40 ppm, about 30 ppm to about 50 ppm, or about 40 ppm to about 50 ppm.

In certain embodiments, biostimulant compositions described herein may be applied to soil, applied to fertilizer used to fertilize plants, applied directly to plants, or applied to both soil and plants. A “fertilizer” as used herein means a compound or composition that is added to plants or soil to improve plant health, growth and/or yield. In some embodiments, a fertilizer improves plant health, growth and/or yield by providing a nutrient (such as the ones described herein) to the plant. Fertilizers can include, but are not limited to, inorganic fertilizers, organic (or natural) fertilizers, granular fertilizers and liquid fertilizers. Granular fertilizers may be solid granules, while liquid fertilizers may be made from water soluble powders or liquid concentrates that mix with water to form a liquid fertilizer solution. In some embodiments, plants can quickly take up most water-soluble fertilizers, while granular fertilizers, in some examples, may need a while to dissolve or decompose before plants can access their nutrients. High-tech granular fertilizers have “slow-release,” “timed-release,” or “controlled-release” properties, synonymous terms meaning that they release their nutrients slowly over a period of time. Organic fertilizer comes from an organic source such as, but not limited to, compost, manure, blood meal, cottonseed meal, feather meal, bird guano, fish meal, crab meal, corn meal, soy meal, or others, as opposed to synthetic sources. There may be also some natural fertilizers that are not organic, such as Greensand, which contain potassium, iron, calcium, and other nutrients. These may be considered suitable for organic gardening because they are not synthesized, but come from natural mineral-rich deposits in the earth. Organic fertilizers depend on the microbes in the soil to break them down into digestible bits for plants. In some embodiments, organic fertilizers encourage soil microbes, earthworms, and other flora more than synthetic fertilizers do, because most organic fertilizers don't add excess salts and acid to the soil. Inorganic fertilizers may be also known as synthetic or artificial fertilizers. Inorganic fertilizers may be manufactured. In some embodiments, the fertilizer can be CircaZn™ fertilizer. Fertilizers can be coated with synthetic, natural, and sulfur polymers. Natural coatings, due to their low cost and abundance in nature, can be a suitable option for use as a coating. Because it is hydrophilic, a material such as lignin may be first acetylated to produce a coating in fertilizers. Other natural polymers that can coat fertilizers may comprise starch, natural rubber, chitosan, cellulose, and alginate. These materials can provide a more consistent release of nutrients.

Compositions may also be applied directly to a plant seed. In addition to soil, biostimulant compositions may be applied to other plant growth media such as, for example, a hydroponic growth medium. Compositions may be used in in-furrow applications, foliar applications, or both. In some embodiments, the biostimulant composition is applied on its own. When applied on its own, in some embodiments, the composition is applied before or after application of a fertilizer. When applied before or after application of a fertilizer, the composition is applied sufficiently close in time to the fertilizer so that the formulation may have its desired effect of enhancing the effect of the fertilizer. In some embodiments, the composition is applied in conjunction with a fertilizer. The composition may either be mixed with a fertilizer or applied simultaneously with a fertilizer.

In some embodiments, the biostimulant compositions described herein may be mixed with a fertilizer at a ratio of at least about 5:1, at least about 4:1, at least about 3:1, at least about 2:1, at least about 1:1, at least about 1:2, at least about 1:3, at least about 1:4, at least about 1:5, at least about 1:10, at least about 1:25, at least about 1:50, at least about 1:75, or at least about 1:100 biostimulant to fertilizer. In some embodiments, the biostimulant compositions described herein may be mixed with a fertilizer at a ratio of at most about 1:100, at most about 1:75, at most about 1:50, at most about 1:25, at most about 1:10, at most about 1:5, at most about 1:4, at most about 1:3, at most about 1:2, at most about 1:1, at most about 2:1, at most about 3:1, at most about 4:1, or at most about 5:1 biostimulant to fertilizer. A biostimulant composition described herein may be mixed with a fertilizer at a ratio of at least about 2:1, 3:1, 4:1, 5:1, 6:1, 7:1, 8:1, 9:1, or 10:1 biostimulant composition to fertilizer. A biostimulant may be mixed with a fertilizer at a range of at least about 0.01%, 0.1%, 0.5%, 1%, 1.5%, 2%, 2.5%, 5%, 6%, 7%, 8%, 9%, 10%, 11%, 12%, 13%, 14%, or 15%. A biostimulant may be mixed with a fertilizer at a range of at most about 0.01%, 0.1%, 0.5%, 1%, 1.5%, 2%, 2.5%, 5%, 6%, 7%, 8%, 9%, 10%, 11%, 12%, 13%, 14%, or 15%. In some embodiments, biostimulant compositions may be mixed with a fertilizer or pesticide in a ratio of about 1:20 or 1:100 biostimulant to fertilizer or pesticide. Biostimulant compositions described herein may also be coated on particles of fertilizer or pesticide. Particles of fertilizer or pesticide may be coated by, for example, spray drying the biostimulant onto the surface of the fertilizer or by mixing a dehydrated powder form of the biostimulant with the particles, with or without a binder or carrier.

In certain embodiments, the fertilizer is a starter fertilizer. In some embodiments, the fertilizer can be ammonia, urea, ammonium nitrate, ammonium sulfate, ammonium thiosulfate, monoammonium phosphate (MAP), diammonium phosphate (DAP), muriate of potash (MOP), sulfate of Potash (SOP), potassium nitrate (NOP), or any combination thereof. In some embodiments, the fertilizer can comprise micronutrients. In some embodiments, the starter fertilizer is a 10-34-0 starter fertilizer. In some embodiments, the fertilizer may be an organic fertilizer (e.g., chicken litter, bird guano, manure, fish meal, and/or hydrolysates), a soy protein based fertilizer, a corn protein based fertilizer (e.g., corn gluten), or any combination thereof. In some embodiments, the fertilizer may be pelletized chicken litter. The fertilizers can contain one or more of calcium (Ca), magnesium (Mg), boron, copper, iron, manganese, zinc, chlorine, cobalt, or sulfur (SO). The fertilizers can be dry or liquids.

In certain embodiments, the biostimulant compositions described herein may be applied to soil or an area of plants in an amount of at least about 0.25, 0.5, 1.0, 1.5, 2.0, 2.5, 3.0, 3.5, 4.0 4.5, 5.0, 6.0, 7.0, 8.0, 9.0, 10.0, 12.0, 15.0, 20.0 quarts per acre of a soil or area of plants (e.g., crops). In certain embodiments, the biostimulant compositions described herein may be applied to soil or an area of plants in an amount of at most about 0.25, 0.5, 1.0, 1.5, 2.0, 2.5, 3.0, 3.5, 4.0 4.5, 5.0, 6.0, 7.0, 8.0, 9.0, 10.0, 12.0, 15.0, 20.0 quarts per acre of a soil or area of plants (e.g., crops).

In certain embodiments, the biostimulant compositions described herein may be applied to soil or plants in an amount per ton of a fertilizer or growth medium, of at least about 5.0, 6.0, 7.0, 8.0, 9.0, 10.0, 12.0, 15.0, 20.0, 25.0, 30.0, 32.0, 40.0, 50.0, 75.0, 100.0, 120.0, 140.0, 160.0, 180.0, 200.0, 250.0, 300.0 ounces per ton (oz/ton). In certain embodiments, the biostimulant compositions described herein may be applied to soil or plants in an amount per ton of a fertilizer or growth medium, of at most about 5.0, 6.0, 7.0, 8.0, 9.0, 10.0, 12.0, 15.0, 20.0, 25.0, 30.0, 32.0, 40.0, 50.0, 75.0, 100.0, 120.0, 140.0, 160.0, 180.0, 200.0, 250.0, 300.0 ounces per ton (oz/ton) In some embodiments, the formulations may be applied in an amount of about 2 quarts per acre of a soil or area of plants (e.g., crops). In some embodiments, the formulations may be applied in an amount of about 4 quarts per acre of a soil or area of plants (e.g., crops). In some embodiments, the biostimulant composition is applied in an amount of about 0.5, 1, 2, 3, 4, 5, 6, 7, 8, 9, or 10 quarts per acre of a soil or area of plants (e.g., crops). In some embodiments, the biostimulant composition may be applied at 64 oz/ton of plant growth medium (e.g., fertilizer). In some embodiments, the biostimulant composition may be applied at 120 oz/ton of plant growth medium (e.g., fertilizer). In some embodiments, the biostimulant composition may be applied at 160 oz/ton of plant growth medium (e.g., fertilizer). In some embodiments, the amount of biostimulant composition applied is characterized by the dry weight of substances present in the biostimulant composition applied. The dry weight of a given volume of liquid biostimulant composition is the weight of all substances in the volume of biostimulant other than water. In some embodiments, an amount of biostimulant is applied that provides for 0.10 to 10 g by dry weight of digestion products to be applied per acre. In some embodiments, the amount of biostimulant applied provides for at least about 0.1, 0.5, 1, 2, 3, 4, 5, 6, 7, 8, 9, or 10 g by dry weight of digestion products per acre. In some embodiments, the amount of biostimulant applied provides for at most about 0.1, 0.5, 1, 2, 3, 4, 5, 6, 7, 8, 9, or 10 g by dry weight of digestion products per acre. In some embodiments, the amount of biostimulant applied provides for at least about 0.1, 0.5, 1, 2, 3, 4, 5, 6, 7, 8, 9, or 10 g by dry weight of biostimulant components to be applied. In some embodiments, the amount of biostimulant applied provides for at most about 0.1, 0.5, 1, 2, 3, 4, 5, 6, 7, 8, 9, or 10 g by dry weight of biostimulant components to be applied. The amount of biostimulant composition applied may also be characterized in terms of the numbers of colony forming units of bacteria applied. In some embodiments, the amount of biostimulant applied provides for at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, or 1×10¹⁰ CFU of bacteria to be applied per acre. In some embodiments, the amount of biostimulant applied provides for at most about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, or 1×10¹⁰ CFU of bacteria to be applied per acre.

In some embodiments, biostimulant compositions described herein may be applied in dry form. A biostimulant base product may be dehydrated to make a powdered product that is applied to a growth medium (e.g., soil), to a plant, or to a seed. The biostimulant composition can be added to fertilizer coatings. The biostimulant composition may be added to fertilizer coatings for any fertilizer (e.g., dry fertilizer and/or liquid fertilizer).

In some embodiments, the amount of biostimulant applied is an effective amount to achieve a desired plant growth promoting effect. For instance, an effective amount of a biostimulant base product, such as a product of a bioreactor system as described herein (e.g., a zinc solubility system, a phosphate solubility system, a nitrogen use efficiency system), to increase plant height (e.g., cotton plant height, corn plant height) in comparison to untreated plants is 0.5 or 1 quarts per acre (qt./A). In some embodiments, an effective amount of a biostimulant base product may be at least about 0.1, 0.2, 0.3, 0.4, 0.5, 0.6, 0.7, 0.8, 0.9, 1.0, 1.25, 1.5, 1.75, 2.0, 2.5, 3.0, 3.5, 4.0, 4.5, 5.0, 5.5, 6.0, 7.0, 8.0, 9.0, or 10.0 qt/A. In some embodiments, an effective amount of a biostimulant base product may be at most about 0.1, 0.2, 0.3, 0.4, 0.5, 0.6, 0.7, 0.8, 0.9, 1.0, 1.25, 1.5, 1.75, 2.0, 2.5, 3.0, 3.5, 4.0, 4.5, 5.0, 5.5, 6.0, 7.0, 8.0, 9.0, or 10.0 qt/A. In some embodiments, the biostimulant composition is applied in an effective amount to increase a plant's tolerance to drought, salt, heat, or cold stresses, or to achieve any other desirable outcome described herein that the biostimulant composition is capable of achieving.

The compositions comprising a biostimulant composition and other components described herein (e.g., a fertilizer) can be formed by mixing the components in a tank (i.e., tank mix). Following mixing, formulations can be bottled or otherwise packaged (e.g., in drums), applied to a field or crop, or mixed with other components. When bottled or otherwise packaged, the end user can mix the formulation with other components prior to application. The biostimulant composition can be mixed with fertilizer by tank mixing, including splash mixing with minimal further mixing, or can be blended into the fertilizer.

In some embodiments, the biostimulant is applied once. In some embodiments, a single application is sufficient to promote plant growth and/or nitrogen use efficiency as described herein. In some embodiments, a biostimulant composition is applied 1, 2, 3, 4, 5, or more times during a growing season. In some embodiments, applications may be 1, 2, 3, 4, 5, 6, or more weeks apart.

In an aspect, the present disclosure may provide a microbial formulation comprising: (a) a Bacillus safensis strain having one or more of the following: (i) a 16S rRNA gene sequence at least 95% identical to SEQ ID NO: 1; (ii) a gyrB gene sequence at least 95% identical to SEQ ID NO: 4; and (iii) a rpoB gene sequence at least 95% identical to SEQ ID NO: 7; and a carrier. In some embodiments, the formulation may comprise additional products of digestion of an organic material by a Bacillus safensis strain. A carrier may be a fertilizer as described herein and the fertilizer may comprise ammonia, urea, ammonium nitrate, ammonium sulfate, ammonium thiosulfate, monoammonium phosphate (MAP), diammonium phosphate (DAP), muriate of potash (MOP), sulfate of Potash (SOP), potassium nitrate (NOP), or any combination thereof. The carrier may be an organic fertilizer such as organic compost, manure, animal droppings, domestic sewage, plant manures, humic acid, grain meal, seaweed extracts, or a combination thereof. A carrier may also be coated by a micronutrient. The micronutrient may be a zinc ion. The carrier of the formulation described herein may be a liquid or a solid. The concentration of the Bacillus safensis strain may be at least about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, or 1×10¹⁰ CFU. The concentration of the Bacillus safensis strain may be at most about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, or 1×10¹⁰ CFU.

In another aspect, the present disclosure may provide an isolated strain of the species Bacillus safensis having one or more of the following: (a) a 16S rRNA gene sequence at least 95% identical to SEQ ID NO: 1; (b) a gyrB gene sequence at least 95% identical to SEQ ID NO: 4; and (c) a rpoB gene sequence at least 95% identical to SEQ ID NO: 7. In some embodiments, a method may be utilized which comprises contacting a plant or a medium with a microbial formulation described herein. Without wishing to be bound by theory, a plant contacted by the microbial formulation described herein may lead to an increase in bioavailable zinc available to the plant. The increase in solubilized zinc may be at least about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%.

The increase in solubilized zinc may be at most about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%.

In an aspect, the present disclosure may provide a microbial formulation comprising: (a) a Bacillus megaterium strain having one or more of the following: (i) a 16S rRNA gene sequence at least 95% identical to SEQ ID NO: 2; (ii) a gyrB gene sequence at least 95% identical to SEQ ID NO: 5; and (iii) a rpoB gene sequence at least 95% identical to SEQ ID NO: 8; and a carrier. In some embodiments, the formulation may comprise additional products of digestion of an organic material by a Bacillus megaterium strain. A carrier may be a fertilizer as described herein and the fertilizer may comprise ammonia, urea, ammonium nitrate, ammonium sulfate, ammonium thiosulfate, monoammonium phosphate (MAP), diammonium phosphate (DAP), muriate of potash (MOP), sulfate of Potash (SOP), potassium nitrate (NOP) or any combination thereof. The carrier may be an organic fertilizer such as organic compost, manure, animal droppings, domestic sewage, plant manures, humic acid, grain meal, seaweed extracts, or a combination thereof. The carrier may be an organic fertilizer such as organic compost, manure, animal droppings, domestic sewage, plant manures, humic acid, grain meal, seaweed extracts, or any combination thereof. A carrier may also be coated by a micronutrient or more than one micronutrient. The micronutrient may be a zinc ion. The carrier of the formulation described herein may be a liquid or a solid. The concentration of the Bacillus megaterium strain may be at least about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, or 1×10¹⁰ CFU. The concentration of the Bacillus megaterium strain may be at most about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, or 1×10¹⁰ CFU.

In another aspect, the present disclosure may provide an isolated strain of the species Bacillus megaterium having one or more of the following: (a) a 16S rRNA gene sequence at least 95% identical to SEQ ID NO: 2; (b) a gyrB gene sequence at least 95% identical to SEQ ID NO: 5; and (c) a rpoB gene sequence at least 95% identical to SEQ ID NO: 8. In some embodiments, a method may be utilized which comprises contacting a plant or a medium with a microbial formulation described herein. Without wishing to be bound by theory, a plant contacted by the microbial formulation described herein may lead to an increase in solubilized zinc available to the plant. The increase in solubilized zinc may be at least about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%. The increase in solubilized zinc may be at most about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%.

In an aspect, the present disclosure may provide a microbial formulation comprising: (a) a Bacillus megaterium strain having one or more of the following: (i) a 16S rRNA gene sequence at least 95% identical to SEQ ID NO: 3; (ii) a gyrB gene sequence at least 95% identical to SEQ ID NO: 6; and (iii) a rpoB gene sequence at least 95% identical to SEQ ID NO: 9; and a carrier. In some embodiments, the formulation may comprise additional products of digestion of an organic material by a Bacillus megaterium strain. A carrier may be a fertilizer as described herein and the fertilizer may comprise ammonia, urea, ammonium nitrate, ammonium sulfate, ammonium thiosulfate, monoammonium phosphate (MAP), diammonium phosphate (DAP), muriate of potash (MOP), sulfate of Potash (SOP), potassium nitrate (NOP) or any combination thereof. The carrier may be an organic fertilizer such as organic compost, manure, animal droppings, domestic sewage, plant manures, humic acid, grain meal, seaweed extracts, or a combination thereof. A carrier may also be coated by a micronutrient or more than one micronutrient. The micronutrient may be a zinc ion. The carrier of the formulation described herein may be a liquid or a solid. The concentration of the Bacillus megaterium strain may be at least about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, or 1×10¹⁰ CFU. The concentration of the Bacillus megaterium strain may be at most about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, or 1×10¹⁰ CFU.

In another aspect, the present disclosure may provide an isolated strain of the species Bacillus megaterium having one or more of the following: (a) a 16S rRNA gene sequence at least 95% identical to SEQ ID NO: 3; (b) a gyrB gene sequence at least 95% identical to SEQ ID NO: 6; and (c) a rpoB gene sequence at least 95% identical to SEQ ID NO: 9. In some embodiments, a method may be utilized which comprises contacting a plant or a medium with a microbial formulation described herein. Without wishing to be bound by theory, a plant contacted by the microbial formulation described herein may lead to an increase in solubilized zinc available to the plant. The increase in solubilized zinc may be at least about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%. The increase in solubilized zinc may be at most about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%.

In some embodiments, any one of the microbial formulations described herein may increase the uptake of zinc by a plant by at least about 1%, 10%, 20%, 30%, 40%, 50%, 55%, 60%, 65%, 70%, 75%, 80%, 85%, 90%, 95%, 99%, 99.5%, or 100%. In some embodiments, any one of the microbial formulations described herein may increase the uptake of zinc by a plant by at most about 1%, 10%, 20%, 30%, 40%, 50%, 55%, 60%, 65%, 70%, 75%, 80%, 85%, 90%, 95%, 99%, 99.5%, or 100%.

In an aspect, the present disclosure may provide a microbial formulation comprising: (a) a Bacillus amyloliquefaciens strain having one or more of the following: (i) a 16S rRNA gene sequence at least 95% identical to SEQ ID NO: 10; (ii) a gyrB gene sequence at least 95% identical to SEQ ID NO: 12; and (iii) a rpoB gene sequence at least 95% identical to SEQ ID NO: 14; and a carrier. In some embodiments, the formulation may comprise additional products of digestion of an organic material by a Bacillus amyloliquefaciens strain. A carrier may be a fertilizer as described herein and the fertilizer may comprise ammonia, urea, ammonium nitrate, ammonium sulfate, ammonium thiosulfate, monoammonium phosphate (MAP), diammonium phosphate (DAP), muriate of potash (MOP), sulfate of Potash (SOP), potassium nitrate (NOP), or any combination thereof. The carrier may be an organic fertilizer such as organic compost, manure, animal droppings, domestic sewage, plant manures, humic acid, grain meal, seaweed extracts, or a combination thereof. A carrier may also be coated by a micronutrient. The micronutrient may be a phosphate ion. The carrier of the formulation described herein may be a liquid or a solid. The concentration of the Bacillus amyloliquefaciens strain may be at least about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, or 1×10¹⁰ CFU. The concentration of the Bacillus amyloliquefaciens strain may be at most about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, or 1×10¹⁰ CFU.

In another aspect, the present disclosure may provide an isolated strain of the species Bacillus amyloliquefaciens having one or more of the following: (a) a 16S rRNA gene sequence at least 95% identical to SEQ ID NO: 10; (b) a gyrB gene sequence at least 95% identical to SEQ ID NO: 12; and (c) a rpoB gene sequence at least 95% identical to SEQ ID NO: 14. In some embodiments, a method may be utilized which comprises contacting a plant or a medium with a microbial formulation described herein. Without wishing to be bound by theory, a plant contacted by the microbial formulation described herein may lead to an increase in solubilized phosphate available to the plant. The increase in solubilized phosphate may be at least about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%. The increase in solubilized phosphate may be at most about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%.

In an aspect, the present disclosure may provide a microbial formulation comprising: (a) a Bacillus licheniformis strain having one or more of the following: (i) a 16S rRNA gene sequence at least 95% identical to SEQ ID NO: 11; (ii) a gyrB gene sequence at least 95% identical to SEQ ID NO: 13; and (iii) a rpoB gene sequence at least 95% identical to SEQ ID NO: 15; and a carrier. In some embodiments, the formulation may comprise additional products of digestion of an organic material by a Bacillus licheniformis strain. A carrier may be a fertilizer as described herein and the fertilizer may comprise ammonia, urea, ammonium nitrate, ammonium sulfate, ammonium thiosulfate, monoammonium phosphate (MAP), diammonium phosphate (DAP), muriate of potash (MOP), sulfate of Potash (SOP), potassium nitrate (NOP), or any combination thereof. The carrier may be an organic fertilizer such as organic compost, manure, animal droppings, domestic sewage, plant manures, humic acid, grain meal, seaweed extracts, or a combination thereof. A carrier may also be coated by a micronutrient. The micronutrient may be a phosphate ion. The carrier of the formulation described herein may be a liquid or a solid. The concentration of the Bacillus licheniformis strain may be at least about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, or 1×10¹⁰ CFU. The concentration of the Bacillus licheniformis strain may be at most about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, or 1×10¹⁰ CFU.

In another aspect, the present disclosure may provide an isolated strain of the species Bacillus licheniformis having one or more of the following: (a) a 16S rRNA gene sequence at least 95% identical to SEQ ID NO: 11; (b) a gyrB gene sequence at least 95% identical to SEQ ID NO: 13; and (c) a rpoB gene sequence at least 95% identical to SEQ ID NO: 15. In some embodiments, a method may be utilized which comprises contacting a plant or a medium with a microbial formulation described herein. Without wishing to be bound by theory, a plant contacted by the microbial formulation described herein may lead to an increase in solubilized phosphate available to the plant. The increase in solubilized phosphate may be at least about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%. The increase in solubilized phosphate may be at most about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%.

In an aspect, the present disclosure may provide a microbial formulation comprising: (a) a Bacillus megaterium strain having one or more of the following: (i) a 16S rRNA gene sequence at least 95% identical to SEQ ID NO: 16; (ii) a gyrB gene sequence at least 95% identical to SEQ ID NO: 20; and (iii) a rpoB gene sequence at least 95% identical to SEQ ID NO: 24; and a carrier. In some embodiments, the formulation may comprise additional products of digestion of an organic material by a Bacillus megaterium strain. A carrier may be a fertilizer as described herein and the fertilizer may comprise, ammonia, urea, ammonium nitrate, ammonium sulfate, ammonium thiosulfate, monoammonium phosphate (MAP), diammonium phosphate (DAP), muriate of potash (MOP), sulfate of Potash (SOP), potassium nitrate (NOP). A carrier may also be coated by a micronutrient. The carrier of the formulation described herein may be a liquid or a solid. The concentration of the Bacillus megaterium strain may be at least about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, 1×10¹⁰ CFU, or 1×10¹¹ CFU. The concentration of the Bacillus megaterium strain may be at most about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, 1×10¹⁰ CFU, or 1×10¹¹ CFU.

In some embodiments, any one of the microbial formulations described herein may increase the uptake of phosphate by a plant by at least about 10%, 15%, 20%, 30%, 40%, 50%, 55%, 60%, 65%, 70%, 75%, 80%, 85%, 90%, 95%, 99%, 99.5%, or 100%. In some embodiments, any one of the microbial formulations described herein may increase the uptake of phosphate by a plant by at most about 10%, 15%, 20%, 30%, 40%, 50%, 55%, 60%, 65%, 70%, 75%, 80%, 85%, 90%, 95%, 99%, 99.5%, or 100%.

In another aspect, the present disclosure may provide an isolated strain of the species Bacillus megaterium having one or more of the following: (a) a 16S rRNA gene sequence at least 95% identical to SEQ ID NO: 16; (b) a gyrB gene sequence at least 95% identical to SEQ ID NO: 20; and (c) a rpoB gene sequence at least 95% identical to SEQ ID NO: 24. In some embodiments, a method may be utilized which comprises contacting a plant or a medium with a microbial formulation described herein. Without wishing to be bound by theory, a plant contacted by the microbial formulation described herein may lead to an increase in nitrogen fixation or a nitrogen use efficiency of the plant.

In an aspect, the present disclosure may provide a microbial formulation comprising: (a) a Paenibacillus borealis strain having one or more of the following: (i) a 16S rRNA gene sequence at least 95% identical to SEQ ID NO: 17; (ii) a gyrB gene sequence at least 95% identical to SEQ ID NO: 21; and (iii) a rpoB gene sequence at least 95% identical to SEQ ID NO: 25; and a carrier. In some embodiments, the formulation may comprise additional products of digestion of an organic material by a Paenibacillus borealis strain. A carrier may be a fertilizer as described herein and the fertilizer may comprise, ammonia, urea, ammonium nitrate, ammonium sulfate, ammonium thiosulfate, monoammonium phosphate (MAP), diammonium phosphate (DAP), muriate of potash (MOP), sulfate of Potash (SOP), potassium nitrate (NOP). A carrier may also be coated by a micronutrient. The carrier of the formulation described herein may be a liquid or a solid. The concentration of the Paenibacillus borealis strain may be at least about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, 1×10¹⁰ CFU, or 1×10¹¹ CFU. The concentration of the Paenibacillus borealis strain may be at most about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, 1×10¹⁰ CFU, or 1×10¹¹ CFU.

In another aspect, the present disclosure may provide an isolated strain of the species Paenibacillus borealis having one or more of the following: (a) a 16S rRNA gene sequence at least 95% identical to SEQ ID NO: 17; (b) a gyrB gene sequence at least 95% identical to SEQ ID NO: 21; and (c) a rpoB gene sequence at least 95% identical to SEQ ID NO: 25. In some embodiments, a method may be utilized which comprises contacting a plant or a medium with a microbial formulation described herein. Without wishing to be bound by theory, a plant contacted by the microbial formulation described herein may lead to an increase in nitrogen fixation or a nitrogen use efficiency of the plant. The increase in solubilized zinc may be at least about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%. The increase in solubilized zinc may be at most about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%.

In an aspect, the present disclosure may provide a microbial formulation comprising: (a) a Paenibacillus sonchi strain having one or more of the following: (i) a 16S rRNA gene sequence at least 95% identical to SEQ ID NO: 18; (ii) a gyrB gene sequence at least 95% identical to SEQ ID NO: 22; and (iii) a rpoB gene sequence at least 95% identical to SEQ ID NO: 26; and a carrier. In some embodiments, the formulation may comprise additional products of digestion of an organic material by a Paenibacillus sonchi strain. A carrier may be a fertilizer as described herein and the fertilizer may comprise, ammonia, urea, ammonium nitrate, ammonium sulfate, ammonium thiosulfate, monoammonium phosphate (MAP), diammonium phosphate (DAP), muriate of potash (MOP), sulfate of Potash (SOP), potassium nitrate (NOP). A carrier may also be coated by a micronutrient. The carrier of the formulation described herein may be a liquid or a solid. The concentration of the Paenibacillus sonchi strain may be at least about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, 1×10¹⁰ CFU, or 1×10¹¹ CFU. The concentration of the Paenibacillus sonchi strain may be at most about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, 1×10¹⁰ CFU, or 1×10¹¹ CFU.

In another aspect, the present disclosure may provide an isolated strain of the species Paenibacillus sonchi having one or more of the following: (a) a 16S rRNA gene sequence at least 95% identical to SEQ ID NO: 18; (b) a gyrB gene sequence at least 95% identical to SEQ ID NO: 22; and (c) a rpoB gene sequence at least 95% identical to SEQ ID NO: 26. In some embodiments, a method may be utilized which comprises contacting a plant or a medium with a microbial formulation described herein. Without wishing to be bound by theory, a plant contacted by the microbial formulation described herein may lead to an increase in nitrogen fixation or a nitrogen use efficiency of the plant. The increase in solubilized zinc may be at least about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%. The increase in solubilized zinc may be at most about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%.

In an aspect, the present disclosure may provide a microbial formulation comprising: (a) a Bacillus megaterium strain having one or more of the following: (i) a 16S rRNA gene sequence at least 95% identical to SEQ ID NO: 19; (ii) a gyrB gene sequence at least 95% identical to SEQ ID NO: 23; and (iii) a rpoB gene sequence at least 95% identical to SEQ ID NO: 27; and a carrier. In some embodiments, the formulation may comprise additional products of digestion of an organic material by a Bacillus megaterium strain. A carrier may be a fertilizer as described herein and the fertilizer may comprise, ammonia, urea, ammonium nitrate, ammonium sulfate, ammonium thiosulfate, monoammonium phosphate (MAP), diammonium phosphate (DAP), muriate of potash (MOP), sulfate of Potash (SOP), potassium nitrate (NOP). A carrier may also be coated by a micronutrient. The carrier of the formulation described herein may be a liquid or a solid. The concentration of the Bacillus megaterium strain may be at least about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, 1×10¹⁰ CFU, or 1×10¹¹ CFU. The concentration of the Bacillus megaterium strain may be at most about 1×10² CFU, 5×10² CFU, 1×10³ CFU, 5×10³ CFU, 1×10⁴ CFU, 5×10⁴ CFU, 1×10⁵ CFU, 5×10⁵ CFU, 1×10⁶ CFU, 5×10⁶ CFU, 1×10⁷ CFU, 5×10⁷ CFU, 1×10⁸ CFU, 5×10⁸ CFU, 1×10⁹ CFU, 5×10⁹ CFU, 1×10¹⁰ CFU, or 1×10¹¹ CFU.

In another aspect, the present disclosure may provide an isolated strain of the species Bacillus megaterium having one or more of the following: (a) a 16S rRNA gene sequence at least 95% identical to SEQ ID NO: 19; (b) a gyrB gene sequence at least 95% identical to SEQ ID NO: 23; and (c) a rpoB gene sequence at least 95% identical to SEQ ID NO: 27. In some embodiments, a method may be utilized which comprises contacting a plant or a medium with a microbial formulation described herein. Without wishing to be bound by theory, a plant contacted by the microbial formulation described herein may lead to an increase in nitrogen fixation or a nitrogen use efficiency of the plant. The increase in solubilized zinc may be at least about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%. The increase in solubilized zinc may be at most about 1%, 2%, 3%, 4%, 5%, 6%, 7%, 8%, 9%, 10%, 12%, 15%, 18%, 20%, 25%, 30%, 35%, 40%, 45%, 50%, 60%, 75%, or 100%.

In some embodiments, any one of the microbial formulations described herein may increase the nitrogen fixation activity in plant tissues by at least about 1%, 2%, 3%, 4%, 5%, 10%, 20%, 30%, 40%, 50%, 55%, 60%, 65%, 70%, 75%, 80%, 85%, 90%, 95%, 99%, 99.5%, or 100%. In some embodiments, any one of the microbial formulations described herein may increase the nitrogen fixation activity in plant tissues by at most about 1%, 2%, 3%, 4%, 5%, 10%, 20%, 30%, 40%, 50%, 55%, 60%, 65%, 70%, 75%, 80%, 85%, 90%, 95%, 99%, 99.5%, or 100%.

In some embodiments, any one of the microbial formulations described herein may increase the plant nitrogen content by at least about 1%, 2%, 3%, 4%, 5%, 10%, 20%, 30%, 40%, 50%, 55%, 60%, 65%, 70%, 75%, 80%, 85%, 90%, 95%, 99%, 99.5%, or 100%. In some embodiments, any one of the microbial formulations described herein may increase the plant nitrogen content by at most about 1%, 2%, 3%, 4%, 5%, 10%, 20%, 30%, 40%, 50%, 55%, 60%, 65%, 70%, 75%, 80%, 85%, 90%, 95%, 99%, 99.5%, or 100%.

In some embodiments, any one of the microbial formulations described herein may increase the population of nitrogen fixing bacteria in the root and root rhizospheres by at least about 1%, 2%, 3%, 4%, 5%, 10%, 20%, 30%, 40%, 50%, 55%, 60%, 65%, 70%, 75%, 80%, 85%, 90%, 95%, 99%, 99.5%, or 100%. In some embodiments, any one of the microbial formulations described herein may increase the population of nitrogen fixing bacteria in the root and root rhizospheres by at most about 1%, 2%, 3%, 4%, 5%, 10%, 20%, 30%, 40%, 50%, 55%, 60%, 65%, 70%, 75%, 80%, 85%, 90%, 95%, 99%, 99.5%, or 100%.

In some embodiments, the biostimulant compositions presented herein may be formulated as a seed treatment. The seeds can be substantially uniformly coated with one or more layers of the microbial compositions disclosed herein using conventional methods of mixing, spraying or any combination thereof through the use of treatment application equipment that is specifically designed and manufactured to accurately, safely, and efficiently apply seed treatment products to seeds. Such equipment uses various types of coating technology such as rotary coaters, drum coaters, fluidized bed techniques, spouted beds, rotary mists or any combination thereof. Liquid seed treatments can be applied via either a spinning “atomizer” disk or a spray nozzle which evenly distributes the seed treatment onto the seed as it moves though the spray pattern. A seed may be mixed or tumbled for an additional period of time to achieve additional treatment distribution and drying. The seeds can be primed or unprimed before coating with the inventive compositions to increase the uniformity of germination and emergence. In an alternative embodiment, a dry formulation (e.g., a powder formulation) can be metered onto the moving seed and allowed to mix until completely distributed. A biostimulant base product may be dehydrated to make a powdered product that is applied to a growth medium (e.g., soil), to a plant, or to a seed. In some embodiments, the biostimulant product can be a lyophilized powder or spray dried powder.

In some other embodiments, the solid or liquid microbial compositions described herein may contain functional agents capable of protecting seeds from the harmful effects of selective herbicides such as activated carbon, nutrients (fertilizers), and other agents capable of improving the germination and quality of the products or any combination thereof. Seed coating methods and compositions that are known in the art can be particularly useful when they are modified by the addition of a microbial composition of the present disclosure.

A variety of additives can be added to the seed treatment formulations comprising the inventive compositions. Binders can be added and may comprise those composed of an adhesive polymer that can be natural or synthetic without phytotoxic effect on the seed to be coated. The binder may be selected from the group consisting of polyvinyl acetates, polyvinyl acetate copolymers, ethylene vinyl acetate (EVA) copolymers, polyvinyl alcohols, polyvinyl alcohol copolymers, celluloses, including ethylcelluloses, methylcelluloses, hydroxymethylcelluloses, hydroxypropylcelluloses and carboxymethylcellulose, polyvinylpyrolidones, polysaccharides, including starch, modified starch, dextrins, maltodextrins, alginate and chitosans, fats, oils, proteins, including gelatin and zeins, gum arabics, shellacs, vinylidene chloride and vinylidene chloride copolymers, calcium lignosulfonates, acrylic copolymers, polyvinylacrylates, polyethylene oxide, acrylamide polymers and copolymers, polyhydroxyethyl acrylate, methylacrylamide monomers, and polychloroprene.

In some specific embodiments, in addition to the microbial cells or spores, the coating can further comprise a layer of adherent. The adherent may be non-toxic, biodegradable, and adhesive. Examples of such materials can include, but are not limited to, polyvinyl acetates, polyvinyl acetate copolymers, polyvinyl alcohols, polyvinyl alcohol copolymers, celluloses, such as methyl celluloses, hydroxymethyl celluloses, and hydroxymethyl propyl celluloses, dextrins, alginates, sugars, molasses, polyvinyl pyrrolidones, polysaccharides, proteins, fats, oils, gum arabics, gelatins, syrups, and starches.

Various additives, such as adherents, dispersants, surfactants, and nutrient and buffer ingredients, can also be comprised in the seed treatment formulation. Other conventional seed treatment additives can include, but are not limited to, coating agents, wetting agents, buffering agents, and polysaccharides. At least one agriculturally acceptable carrier can be added to the seed treatment formulation such as water, solids or dry powders. The dry powders can be derived from a variety of materials such as calcium carbonate, gypsum, vermiculite, talc, humus, activated charcoal, and various phosphorous compounds.

In some embodiments, a seed coating composition can comprise at least one filler which is an organic or inorganic, natural or synthetic component with which the active components may be combined to facilitate its application onto the seed. A filler may comprise an inert solid such as clays, natural or synthetic silicates, silica, resins, waxes, solid fertilizers (for example ammonium salts), natural soil minerals, such as kaolins, clays, talc, lime, quartz, attapulgite, montmorillonite, bentonite or diatomaceous earths, or synthetic minerals, such as silica, alumina or silicates, in particular aluminum or magnesium silicates.

A seed treatment formulation may comprise one or more of the following ingredients: other pesticides, including compounds that act below the ground; fungicides, such as captan, thiram, metalaxyl, fludioxonil, oxadixyl, and isomers of each of those materials, and the like; herbicides, including compounds selected from glyphosate, carbamates, thiocarbamates, acetamides, triazines, dinitroanilines, glycerol ethers, pyridazinones, uracils, phenoxys, ureas, and benzoic acids; herbicidal safeners such as benzoxazine, benzhydryl derivatives, N,N-diallyl dichloroacetamide, various dihaloacyl, oxazolidinyl and thiazolidinyl compounds, ethanone, naphthalic anhydride compounds, and oxime derivatives; chemical fertilizers; biological fertilizers, or any combination thereof. The seed treatment formulation may also comprise macronutrients and/or micronutrients. These ingredients may be added as a separate layer on the seed or alternatively may be added as part of a seed coating using a microbial composition of the present disclosure.

The amount of the microbial composition or other ingredients used in the seed treatment may not inhibit germination of the seed, or cause phytotoxic damage to the seed.

The formulation that is used to treat the seed can be in the form of a suspension; emulsion; slurry of particles in an aqueous medium (e.g., water); wettable powder; wettable granules (dry flowable); and dry granules. If formulated as a suspension or slurry, the concentration of the active ingredient in the formulation may be about 0.5% to about 99% by weight (w/w), or may be about 5-40%. If formulated as a suspension or slurry, the concentration of the active ingredient in the formulation may be at least about 0.1%, 0.5%, 1%, 2%, 3%, 4%, 5%, 10%, 15%, 20%, 25%, 30%, 35%, 40%, 45%, 50%, 60%, 70%, 80%, 90%, 95%, 96%, 97%, 98%, 99%, 99.5%, or 99.9%. If formulated as a suspension or slurry, the concentration of the active ingredient in the formulation may be at most about 0.1%, 0.5%, 1%, 2%, 3%, 4%, 5%, 10%, 15%, 20%, 25%, 30%, 35%, 40%, 45%, 50%, 60%, 70%, 80%, 90%, 95%, 96%, 97%, 98%, 99%, 99.5%, or 99.9%.

Other conventional inactive or inert ingredients can be incorporated into the formulation. Such inert ingredients comprise but are not limited to: conventional sticking agents; dispersing agents such as methylcellulose, for example, serve as combined dispersant/sticking agents for use in seed treatments; polyvinyl alcohol; lecithin, polymeric dispersants (e.g., polyvinylpyrrolidone/vinyl acetate); thickeners (e.g., clay thickeners to improve viscosity and reduce settling of particle suspensions); emulsion stabilizers; surfactants; antifreeze compounds (e.g., urea), dyes, colorants, and the like.

Coating formulations can be applied to seeds by a variety of methods, including, but not limited to, mixing in a container (e.g., a bottle or bag), mechanical application, tumbling, spraying, and immersion. A variety of active or inert material can be used for contacting seeds with microbial compositions according to the present disclosure, such as conventional film-coating materials including but not limited to water-based film coating materials such as SEPIRET™ (Seppic, Inc., N.J.) and OPACOAT™ (Berwind Pharm. Services, P.A.)

The amount of a microbial composition described herein that may be used for the treatment of the seed will vary depending upon the type of seed and the type of active ingredients, but the treatment will comprise contacting the seeds with an agriculturally effective amount of the inventive composition. An effective amount may refer to an amount of a composition that may be sufficient to affect beneficial or desired results. An effective amount can be administered in one or more administrations.

In addition to the coating layer, the seed may be treated with one or more of the following ingredients: other pesticides including fungicides and herbicides; herbicidal safeners; and/or fertilizers. These ingredients may be added as a separate layer or alternatively may be added in the coating layer. The microorganism-treated seeds may also be enveloped with a film overcoating to protect the coating.

A plant seed capable of germinating to form a plant can be treated with microbial compositions or formulations of the present disclosure. Suitable seeds may comprise those of cereals, coffee, cole crops, fiber crops, flowers, fruits, legume, oil crops, trees, tuber crops, vegetables, as well as other plants of the monocotyledonous, dicotyledonous species, or any combination thereof. Crop seeds that may be coated may comprise bean, carrot, corn, cotton, grasses, lettuce, peanut, pepper, potato, rapeseed, rice, rye, sorghum, soybean, sugar beet, sunflower, tobacco, tomato seeds, or any combination thereof. For example, barley or wheat (e.g., spring wheat or winter wheat) seeds may be coated with the present microbial compositions. Additionally, for example, corn seeds, soybean seeds, sorghum seeds, or any combination thereof may be coated with the present microbial compositions.

### VI. Methods of Promoting Plant Growth

Embodiments of biostimulant compositions may be used in methods of promoting plant growth for a variety of different plants and conditions. In some embodiments, contacting a plant, seed, or growth medium with the biostimulant promotes plant growth by, for example, increasing growth rate, yield at harvest, production, stem thickness, fruit abundance and/or size, grain production, leaf surface area, root surface area, root length, root depth, shoot thickness, total biomass, or any combination thereof, as compared to a plant that has not received the treatment. A control plant may include, but is not limited to, (a) a plant which is genetically identical to the subject plant but which is not exposed to the same treatment (e.g., inoculant treatment) as the subject plant or (b) the subject plant itself, under conditions in which it has not been exposed to a particular treatment such as, for example, an inoculant or combination of inoculants and/or other chemicals. In some embodiments, a population of the microbial strain (e.g., a target isolate) or a biostimulant composition described herein may improve a property of plant growth. A property of plant growth (e.g., plant growth promotion property) may comprise shoot biomass, root biomass, nutrient uptake, crop yield, deaminase activity, acid production, leaf area, total biomass, nitrogen fixation, phosphate solubilization, zinc solubilization, nitrogen use efficiency, photosynthetic activity, chlorophyll content, or plant height. In some embodiments, promoting plant growth comprises one or more of enhancing seed germination, enhancing early plant development, increasing nutrient uptake, mitigating transplant shock, improving plant reproduction, and improving soil microbial activity.

Zinc can be an important nutrient in plant health. An increase in the bioavailability of zinc may provide benefits to a plant's health and/or growth. In some embodiments, a plant or plant growth medium contacted with the plant growth promoting product (e.g., biostimulant composition) described herein may cause an increase in the amount of solubilized zinc available to the plant. In some embodiments, a plant or plant growth medium contacted with the plant growth promoting product (e.g., biostimulant composition) described herein may increase the amount of solubilized zinc available to the plant by at least about 2%, at least about 5%, at least about 8%, at least about 10%, at least about 12%, at least about 15%, at least about 20%, at least about 25%, at least about 30%, at least about 35%, at least about 40%, at least about 50%, at least about 60%, or at least about 75% as compared to a control (e.g., a plant not contacted by the biostimulant composition). In some embodiments, a plant or plant growth medium contacted with the plant growth promoting product (e.g., biostimulant composition) described herein may increase the amount of solubilized zinc available to the plant by at most about 75%, at most about 60%, at most about 50%, at most about 40%, at most about 35%, at most about 30%, at most about 25%, at most about 20%, at most about 15%, at most about 12%, at most about 10%, at most about 8%, at most about 5%, or at most about 2% as compared to a control plant. In some embodiments, a plant or plant growth medium contacted with the plant growth promoting product (e.g., biostimulant composition) described herein may increase the amount of solubilized zinc available to the plant from about 1% to about 50% as compared to a control (e.g., a plant not treated or contacted with the biostimulant composition). In some embodiments, a plant or plant growth medium contacted with the plant growth promoting product (e.g., biostimulant composition) described herein may increase the amount of solubilized zinc available to the plant from about 1% to about 2%, about 1% to about 3%, about 1% to about 4%, about 1% to about 5%, about 1% to about 10%, about 1% to about 15%, about 1% to about 20%, about 1% to about 25%, about 1% to about 30%, about 1% to about 40%, about 1% to about 50%, about 2% to about 3%, about 2% to about 4%, about 2% to about 5%, about 2% to about 10%, about 2% to about 15%, about 2% to about 20%, about 2% to about 25%, about 2% to about 30%, about 2% to about 40%, about 2% to about 50%, about 3% to about 4%, about 3% to about 5%, about 3% to about 10%, about 3% to about 15%, about 3% to about 20%, about 3% to about 25%, about 3% to about 30%, about 3% to about 40%, about 3% to about 50%, about 4% to about 5%, about 4% to about 10%, about 4% to about 15%, about 4% to about 20%, about 4% to about 25%, about 4% to about 30%, about 4% to about 40%, about 4% to about 50%, about 5% to about 10%, about 5% to about 15%, about 5% to about 20%, about 5% to about 25%, about 5% to about 30%, about 5% to about 40%, about 5% to about 50%, about 10% to about 15%, about 10% to about 20%, about 10% to about 25%, about 10% to about 30%, about 10% to about 40%, about 10% to about 50%, about 15% to about 20%, about 15% to about 25%, about 15% to about 30%, about 15% to about 40%, about 15% to about 50%, about 20% to about 25%, about 20% to about 30%, about 20% to about 40%, about 20% to about 50%, about 25% to about 30%, about 25% to about 40%, about 25% to about 50%, about 30% to about 40%, about 30% to about 50%, or about 40% to about 50% as compared to a control (e.g., a plant not treated or contacted with the biostimulant composition).

Treatment with a population of a microbial strain as described herein (e.g., a target isolate) or a biostimulant composition described herein may increase a level of zinc uptake, quantified as ug of zinc per plant, compared to a level of zinc uptake from an untreated control or a control plant not treated with a population of a microbial strain as described herein (e.g., a target isolate) or a biostimulant composition described herein. In some embodiments, a plant treated with a population of a microbial strain as described herein (e.g., a target isolate) or a biostimulant composition described herein may have a zinc uptake of a crop of at least about 5.0 ug/plant, 5.1 μg/plant, 5.15 μg/plant, 5.2 μg/plant, 5.25 μg/plant, 5.3 μg/plant, 5.35 μg/plant, 5.4 μg/plant, 5.45 μg/plant, 5.5 μg/plant, 5.55 μg/plant, 5.6 μg/plant, 5.65 μg/plant, 5.7 μg/plant, 5.75 μg/plant, 5.8 μg/plant, 5.9 μg/plant, 6.0 μg/plant, 7.0 μg/plant, 8.0 μg/plant, 9.0 μg/plant, 10.0 μg/plant. In some embodiments, a plant treated with a population of a microbial strain as described herein (e.g., a target isolate) or a biostimulant composition described herein may have a zinc uptake of a crop of at most about 10.0 μg/plant. 9.0 μg/plant, 8.0 μg/plant, 7.0 μg/plant, 6.0 μg/plant, 5.9 μg/plant, 5.8 μg/plant, 5.75 μg/plant, 5.7 μg/plant, 5.65 μg/plant, 5.6 μg/plant, 5.55 μg/plant, 5.5 μg/plant, 5.45 μg/plant, 5.4 μg/plant, 5.35 μg/plant, 5.3 μg/plant, 5.25 μg/plant, 5.2 μg/plant, 5.15 μg/plant, 5.1 μg/plant, 5.0 μg/plant. Zinc uptake may also be provided in units of ug of Zn per gram of dry weight of plant tissue. In some embodiments, uptake can be at least about 0.001, 0.005, 0.01, 0.05, 0.1, 0.2, 0.3, 0.4, 0.5, 0.6, 0.7, 0.8, 0.9, or 1 μg/g of dry weight of plant tissue. In some embodiments, uptake can be at most about 1 0.9, 0.8, 0.7, 0.6, 0.5, 0.4, 0.3, 0.2, 0.1, 0.05, 0.01, 0.005, 0.001 μg/g of dry weight of plant tissue. In some embodiments, treatment with a population of the microbial strain (e.g., a target isolate) or a biostimulant composition described herein may increase a zinc uptake in a plant by at least about 5%, 5.5%, 6%, 6.5%, 7%, 7.5%, 8%, 8.5%, 9%, 9.5%, 10%, 10.5%, 11%, 11.5%, 12%, 12.5%, 13%, 14%, 15%, 20%, or greater than about 20%, as compared to a zinc uptake in an untreated control plant. In some embodiments, treatment with a population of the microbial strain (e.g., a target isolate) or a biostimulant composition described herein may increase a zinc uptake in a plant by at most about 20%, 15%, 14%, 13%, 12.5%, 12%, 11.5%, 11%, 10.5%, 10%, 9.5%, 9%, 8.5%, 8%, 7.5%, 7%, 6.5%, 6%, 5.5%, 5%, or less than about 5%, as compared to a zinc uptake in an untreated control plant.

A biostimulant composition described herein may have greater solubilization capacity of phosphate over time compared to control solutions that do not comprise phosphate solubilizing microbes described herein. In some embodiments, a fertilizer, soil, media, or any combination thereof treated with a population of the microbial strain (e.g., a target isolate) or a biostimulant composition described herein may show an increased amount of phosphate made soluble (e.g., PO₄ mg/L made soluble) compared to an amount from an untreated a fertilizer, soil, media or a fertilizer, soil, media treated with water. In some embodiments, a phosphate solubilization capacity of a fertilizer, soil, plant, or media treated with the microbial strain or a biostimulant composition described herein may be at least about 2%, at least about 5%, at least about 8%, at least about 10%, at least about 12%, at least about 15%, at least about 20%, at least about 30%, at least about 40%, at least about 50%, at least about 60%, at least about 70%, at least about 80%, at least about 90%, at least about 100%, or greater than 100% higher compared to a capacity measured from an untreated control or a fertilizer, soil, plant, or media treated with water. In some embodiments, a phosphate solubilization capacity of a fertilizer, soil, plant, or media treated with the microbial strain or a biostimulant composition described herein may be at most about 100%, at most about 90%, 80%, 70%, 60%, 50%, 40%, 30%, 20%<15%, 10%, 8%, 5%, 2%, or less than about 2% higher compared to a capacity measured from an untreated control or a fertilizer, soil, plant, or media treated with water. In some embodiments, a phosphate solubilization capacity of a fertilizer, soil, plant, or media treated with the microbial strain or a biostimulant composition described herein may be from about 1% to about 100% higher compared to a capacity measured from an untreated control or a fertilizer, soil, plant, or media treated with water. In some embodiments, a phosphate solubilization capacity of a fertilizer, soil, plant, or media treated with the microbial strain or a biostimulant composition described herein may be from about 1% to about 2%, about 1% to about 5%, about 1% to about 8%, about 1% to about 10%, about 1% to about 15%, about 1% to about 20%, about 1% to about 30%, about 1% to about 40%, about 1% to about 50%, about 1% to about 75%, about 1% to about 100%, about 2% to about 5%, about 2% to about 8%, about 2% to about 10%, about 2% to about 15%, about 2% to about 20%, about 2% to about 30%, about 2% to about 40%, about 2% to about 50%, about 2% to about 75%, about 2% to about 100%, about 5% to about 8%, about 5% to about 10%, about 5% to about 15%, about 5% to about 20%, about 5% to about 30%, about 5% to about 40%, about 5% to about 50%, about 5% to about 75%, about 5% to about 100%, about 8% to about 10%, about 8% to about 15%, about 8% to about 20%, about 8% to about 30%, about 8% to about 40%, about 8% to about 50%, about 8% to about 75%, about 8% to about 100%, about 10% to about 15%, about 10% to about 20%, about 10% to about 30%, about 10% to about 40%, about 10% to about 50%, about 10% to about 75%, about 10% to about 100%, about 15% to about 20%, about 15% to about 30%, about 15% to about 40%, about 15% to about 50%, about 15% to about 75%, about 15% to about 100%, about 20% to about 30%, about 20% to about 40%, about 20% to about 50%, about 20% to about 75%, about 20% to about 100%, about 30% to about 40%, about 30% to about 50%, about 30% to about 75%, about 30% to about 100%, about 40% to about 50%, about 40% to about 75%, about 40% to about 100%, about 50% to about 75%, about 50% to about 100%, or about 75% to about 100%.

In some embodiments, contacting a plant (e.g., root, stem, or leaf), seed, or growth medium with the biostimulant promotes plant growth by, for example, increasing a plant's nitrogen use efficiency. In some embodiments, treatment with a biostimulant described herein reduces the amount of nitrogen required for a crop to produce the same yield. In some embodiments, treatment with a biostimulant described herein increases the yield of a crop while keeping the amount of nitrogen provided to the crop stable. For instance, application of a biostimulant described herein and 80% of the amount of nitrogen typically provided to a crop may provide the same or higher yield as providing 100% of the typical nitrogen amount without the biostimulant. In addition, application of a biostimulant described herein and 100% of the typical nitrogen amount may provide a higher crop yield than application of 100% of the typical nitrogen amount without the biostimulant.

In some embodiments, a population of a microbial strain as described herein (e.g., a target isolate) or a biostimulant composition described herein may improve a property of plant growth. A property of plant growth (e.g., plant growth promotion property) may comprise shoot biomass, root biomass, nutrient uptake, crop yield, photosynthetic activity, deaminase activity, acid production, leaf area, total biomass, nitrogen fixation, nitrogen use efficiency, chlorophyll content, or plant height. In some embodiments, a population of a microbial strain as described herein (e.g., a target isolate) or a biostimulant composition described herein may be capable of promoting nitrogen use efficiency in a plant. In some cases, the inoculum of the microbe can be configured to improve nitrogen uptake in a plant. In some embodiments, a biostimulant composition comprising the inoculum of a microbe described herein may be capable of promoting nitrogen use efficiency.

Shoot biomass may be an important characteristic of plant growth, wherein a healthy plant may have a heavier shoot. In some embodiments, a heavier shoot (e.g., greater shoot biomass) may indicate better plant growth. In some embodiments, a lighter shoot (e.g., lower shoot biomass) may indicate better plant growth. In some embodiments, a population of the microbial strain (e.g., a target isolate) or a biostimulant composition described herein may increase a shoot biomass of a plant treated by the microbe (e.g., a target isolate) or the biostimulant composition by at least about 5%, at least about 10%, at least about 12%, at least about 15%, at least about 18%, at least about 20%, at least about 25%, at least about 30%, at least about 35%, at least about 40%, or at least about 50% compared to that of an untreated control plant. In some embodiments, a population of the microbial strain (e.g., a target isolate) or a biostimulant composition described herein may increase a shoot biomass of a plant treated by the microbe (e.g., a target isolate) or the biostimulant composition by at most about 50%, at most about 40%, at most about 35%, at most about 30%, at most about 25%, at most about 20%, at most about 18%, at most about 15%, at most about 12%, at most about 10%, or at most about 5% compared to that of an untreated control plant. In some embodiments, a population of the microbial strain (e.g., a target isolate) or a biostimulant composition described herein may increase a shoot biomass of a plant treated by the microbe (e.g., a target isolate) or the biostimulant composition from about 1% to about 50% compared to that of an control plant. A control plant can comprise a plant administered water, a plant administered no treatment, or a plant administered a standard fertilizer. In some embodiments, a population of the microbial strain (e.g., a target isolate) or a biostimulant composition described herein may increase a shoot biomass of a plant treated by the microbe (e.g., a target isolate) or the biostimulant composition from about 1% to about 2%, about 1% to about 5%, about 1% to about 8%, about 1% to about 10%, about 1% to about 12%, about 1% to about 15%, about 1% to about 18%, about 1% to about 20%, about 1% to about 30%, about 1% to about 40%, about 1% to about 50%, about 2% to about 5%, about 2% to about 8%, about 2% to about 10%, about 2% to about 12%, about 2% to about 15%, about 2% to about 18%, about 2% to about 20%, about 2% to about 30%, about 2% to about 40%, about 2% to about 50%, about 5% to about 8%, about 5% to about 10%, about 5% to about 12%, about 5% to about 15%, about 5% to about 18%, about 5% to about 20%, about 5% to about 30%, about 5% to about 40%, about 5% to about 50%, about 8% to about 10%, about 8% to about 12%, about 8% to about 15%, about 8% to about 18%, about 8% to about 20%, about 8% to about 30%, about 8% to about 40%, about 8% to about 50%, about 10% to about 12%, about 10% to about 15%, about 10% to about 18%, about 10% to about 20%, about 10% to about 30%, about 10% to about 40%, about 10% to about 50%, about 12% to about 15%, about 12% to about 18%, about 12% to about 20%, about 12% to about 30%, about 12% to about 40%, about 12% to about 50%, about 15% to about 18%, about 15% to about 20%, about 15% to about 30%, about 15% to about 40%, about 15% to about 50%, about 18% to about 20%, about 18% to about 30%, about 18% to about 40%, about 18% to about 50%, about 20% to about 30%, about 20% to about 40%, about 20% to about 50%, about 30% to about 40%, about 30% to about 50%, or about 40% to about 50%, as compared to that of a control plant. In some embodiments, a population of the microbial strain (e.g., a target isolate) or a biostimulant composition described herein may increase a shoot biomass of a plant treated by the microbe (e.g., a target isolate) or the biostimulant composition from about 1%, about 2%, about 5%, about 8%, about 10%, about 12%, about 15%, about 18%, about 20%, about 30%, about 40%, or about 50% compared to that of a control plant.

Root biomass may be an important characteristic of plant growth, wherein a healthy plant may have a larger root (e.g., heavier root and greater root biomass). In some embodiments, a heavier root (e.g., greater root biomass) may indicate better plant growth. In some embodiments, a lighter root (e.g., lower root biomass) may indicate better plant growth. In some embodiments, a population of the microbial strain (e.g., a target isolate) or a biostimulant composition described herein may increase a root biomass of a plant treated by the microbe (e.g., a target isolate) or the biostimulant composition by at least about 5%, at least about 10%, at least about 12%, at least about 15%, at least about 18%, at least about 20%, at least about 25%, at least about 30%, at least about 35%, at least about 40%, or at least about 50% compared to that of an untreated control plant. In some embodiments, a population of the microbial strain (e.g., a target isolate) or a biostimulant composition described herein may increase a root biomass of a plant treated by the microbe (e.g., a target isolate) or the biostimulant composition by at most about 50%, at most about 40%, at most about 35%, at most about 30%, at most about 25%, at most about 20%, at most about 18%, at most about 15%, at most about 12%, at most about 10%, or at most about 5% compared to that of an untreated control plant. In some embodiments, a population of the microbial strain (e.g., a target isolate) or a biostimulant composition described herein may increase a root biomass of a plant treated by the microbe (e.g., a target isolate) or the biostimulant composition from about 1% to about 50% compared to that of an untreated control plant. In some embodiments, a population of the microbial strain (e.g., a target isolate) or a biostimulant composition described herein may increase a root biomass of a plant treated by the microbe (e.g., a target isolate) or the biostimulant composition from about 1% to about 2%, about 1% to about 5%, about 1% to about 8%, about 1% to about 10%, about 1% to about 12%, about 1% to about 15%, about 1% to about 18%, about 1% to about 20%, about 1% to about 30%, about 1% to about 40%, about 1% to about 50%, about 2% to about 5%, about 2% to about 8%, about 2% to about 10%, about 2% to about 12%, about 2% to about 15%, about 2% to about 18%, about 2% to about 20%, about 2% to about 30%, about 2% to about 40%, about 2% to about 50%, about 5% to about 8%, about 5% to about 10%, about 5% to about 12%, about 5% to about 15%, about 5% to about 18%, about 5% to about 20%, about 5% to about 30%, about 5% to about 40%, about 5% to about 50%, about 8% to about 10%, about 8% to about 12%, about 8% to about 15%, about 8% to about 18%, about 8% to about 20%, about 8% to about 30%, about 8% to about 40%, about 8% to about 50%, about 10% to about 12%, about 10% to about 15%, about 10% to about 18%, about 10% to about 20%, about 10% to about 30%, about 10% to about 40%, about 10% to about 50%, about 12% to about 15%, about 12% to about 18%, about 12% to about 20%, about 12% to about 30%, about 12% to about 40%, about 12% to about 50%, about 15% to about 18%, about 15% to about 20%, about 15% to about 30%, about 15% to about 40%, about 15% to about 50%, about 18% to about 20%, about 18% to about 30%, about 18% to about 40%, about 18% to about 50%, about 20% to about 30%, about 20% to about 40%, about 20% to about 50%, about 30% to about 40%, about 30% to about 50%, or about 40% to about 50%. In some embodiments, a population of the microbial strain (e.g., a target isolate) or a biostimulant composition described herein may increase a shoot of a plant treated by the microbe (e.g., a target isolate) or the biostimulant composition biomass from about 1%, about 2%, about 5%, about 8%, about 10%, about 12%, about 15%, about 18%, about 20%, about 30%, about 40%, or about 50% compared to that of an untreated control plant.

Total plant biomass may be an important characteristic of plant growth, wherein a healthy plant may have a larger plant (e.g., a heavier plant with a greater plant biomass). In some embodiments, a heavier total biomass (e.g., greater total biomass) may indicate better plant growth. In some embodiments, a lighter total biomass (e.g., lower total biomass) may indicate better plant growth. In some embodiments, a population of the microbial strain (e.g., a target isolate) or a biostimulant composition described herein may increase a total biomass of a plant treated by the microbe (e.g., a target isolate) or the biostimulant composition by at least about 5%, at least about 10%, at least about 12%, at least about 15%, at least about 18%, at least about 20%, at least about 25%, at least about 30%, at least about 35%, at least about 40%, at least about 50%, at least about 60%, or at least about 75% compared to that of an untreated control plant. In some embodiments, a population of the microbial strain (e.g., a target isolate) or a biostimulant composition described herein may increase a total biomass of a plant treated by the microbe (e.g., a target isolate) or the biostimulant composition by at most about 75%, at most about 60%, at most about 50%, at most about 40%, at most about 35%, at most about 30%, at most about 25%, at most about 20%, at most about 18%, at most about 15%, at most about 12%, at most about 10%, or at most about 5% compared to that of an untreated control plant. In some embodiments, a population of the microbial strain (e.g., a target isolate) or a biostimulant composition described herein may increase a total biomass of a plant treated by the microbe (e.g., a target isolate) or the biostimulant composition from about 3% to about 75% compared to that of an untreated control plant. In some embodiments, a population of the microbial strain (e.g., a target isolate) or a biostimulant composition described herein may increase a total biomass of a plant treated by the microbe (e.g., a target isolate) or the biostimulant composition from about 3% to about 5%, about 3% to about 8%, about 3% to about 10%, about 3% to about 12%, about 3% to about 15%, about 3% to about 20%, about 3% to about 25%, about 3% to about 30%, about 3% to about 40%, about 3% to about 50%, about 3% to about 75%, about 5% to about 8%, about 5% to about 10%, about 5% to about 12%, about 5% to about 15%, about 5% to about 20%, about 5% to about 25%, about 5% to about 30%, about 5% to about 40%, about 5% to about 50%, about 5% to about 75%, about 8% to about 10%, about 8% to about 12%, about 8% to about 15%, about 8% to about 20%, about 8% to about 25%, about 8% to about 30%, about 8% to about 40%, about 8% to about 50%, about 8% to about 75%, about 10% to about 12%, about 10% to about 15%, about 10% to about 20%, about 10% to about 25%, about 10% to about 30%, about 10% to about 40%, about 10% to about 50%, about 10% to about 75%, about 12% to about 15%, about 12% to about 20%, about 12% to about 25%, about 12% to about 30%, about 12% to about 40%, about 12% to about 50%, about 12% to about 75%, about 15% to about 20%, about 15% to about 25%, about 15% to about 30%, about 15% to about 40%, about 15% to about 50%, about 15% to about 75%, about 20% to about 25%, about 20% to about 30%, about 20% to about 40%, about 20% to about 50%, about 20% to about 75%, about 25% to about 30%, about 25% to about 40%, about 25% to about 50%, about 25% to about 75%, about 30% to about 40%, about 30% to about 50%, about 30% to about 75%, about 40% to about 50%, about 40% to about 75%, or about 50% to about 75% compared to that of an untreated control plant.

Nutrient uptake may be an important characteristic of plant growth, wherein a healthy plant may have a greater nutrient uptake. In some embodiments, a greater nutrient uptake may indicate better plant growth. In some embodiments, a lower nutrient uptake may indicate better plant growth. In some embodiments, a population of the microbial strain (e.g., a target isolate) or a biostimulant composition described herein may increase a level of nutrient uptake. In some embodiments, a plant may be treated by a population of the microbial strain (e.g., a target isolate) or a biostimulant composition described herein. The nutrients may comprise sulfur, nitrogen, potassium, phosphorus, magnesium, calcium, zinc, boron, copper, iron, manganese, or any combination thereof. In some embodiments, a population of the microbial strain (e.g., a target isolate) or a biostimulant composition described herein may increase a level of nutrient uptake of a plant treated by the microbe (e.g., a target isolate) or the biostimulant composition by at least about 0.5%, at least about 1%, at least about 1.5%, at least about 2%, at least about 3%, at least about 5%, at least about 10%, at least about 12%, at least about 15%, at least about 18%, at least about 20%, at least about 25%, at least about 30%, at least about 35%, at least about 40%, at least about 50%, at least about 60%, or at least about 75% compared to that of an untreated control plant. In some embodiments, a population of the microbial strain (e.g., a target isolate) or a biostimulant composition described herein may increase a level of nutrient uptake of a plant treated by the microbe (e.g., a target isolate) or the biostimulant composition by at most about 75%, at most about 60%, at most about 50%, at most about 40%, at most about 35%, at most about 30%, at most about 25%, at most about 20%, at most about 18%, at most about 15%, at most about 12%, at most about 10%, at most about 5%, at most about 3%, at most about 2%, at most about 1.5%, at most about 1%, or at most about 0.5% compared to that of an untreated control plant. In some embodiments, a population of the microbial strain (e.g., a target isolate) or a biostimulant composition described herein may increase a level of nutrient uptake of a plant treated by the microbe (e.g., a target isolate) or the biostimulant composition from about 0.1% to about 50% compared to that of an untreated control plant. In some embodiments, a population of the microbial strain (e.g., a target isolate) or a biostimulant composition described herein may increase a level of nutrient uptake of a plant treated by the microbe (e.g., a target isolate) or the biostimulant composition from about 0.1% to about 0.2%, about 0.1% to about 0.5%, about 0.1% to about 1%, about 0.1% to about 2%, about 0.1% to about 3%, about 0.1% to about 5%, about 0.1% to about 10%, about 0.1% to about 15%, about 0.1% to about 20%, about 0.1% to about 25%, about 0.1% to about 50%, about 0.2% to about 0.5%, about 0.2% to about 1%, about 0.2% to about 2%, about 0.2% to about 3%, about 0.2% to about 5%, about 0.2% to about 10%, about 0.2% to about 15%, about 0.2% to about 20%, about 0.2% to about 25%, about 0.2% to about 50%, about 0.5% to about 1%, about 0.5% to about 2%, about 0.5% to about 3%, about 0.5% to about 5%, about 0.5% to about 10%, about 0.5% to about 15%, about 0.5% to about 20%, about 0.5% to about 25%, about 0.5% to about 50%, about 1% to about 2%, about 1% to about 3%, about 1% to about 5%, about 1% to about 10%, about 1% to about 15%, about 1% to about 20%, about 1% to about 25%, about 1% to about 50%, about 2% to about 3%, about 2% to about 5%, about 2% to about 10%, about 2% to about 15%, about 2% to about 20%, about 2% to about 25%, about 2% to about 50%, about 3% to about 5%, about 3% to about 10%, about 3% to about 15%, about 3% to about 20%, about 3% to about 25%, about 3% to about 50%, about 5% to about 10%, about 5% to about 15%, about 5% to about 20%, about 5% to about 25%, about 5% to about 50%, about 10% to about 15%, about 10% to about 20%, about 10% to about 25%, about 10% to about 50%, about 15% to about 20%, about 15% to about 25%, about 15% to about 50%, about 20% to about 25%, about 20% to about 50%, or about 25% to about 50% compared to that of an untreated control plant.

Crop yield may be an important characteristic of plant growth, wherein healthy plants may produce more crops. In some embodiments, a greater crop yield may indicate better plant growth. In some embodiments, a population of the microbial strain (e.g., a target isolate) or a biostimulant composition described herein may increase a crop yield. Crops may comprise corn, grains, cotton, fruits, tree nuts, rice, soybeans, oil crops, sugar, vegetables, pulses, wheat, or any combination thereof. In some embodiments, a population of the microbial strain (e.g., a target isolate) or a biostimulant composition described herein may increase a crop yield of a plant treated by the microbe (e.g., a target isolate) or the biostimulant composition by at least about 0.5%, at least about 1%, at least about 1.5%, at least about 2%, at least about 3%, at least about 5%, at least about 10%, at least about 12%, at least about 15%, at least about 18%, at least about 20%, at least about 25%, at least about 30%, at least about 35%, at least about 40%, at least about 50%, at least about 60%, or at least about 75% compared to that of an untreated control plant. In some embodiments, a population of the microbial strain (e.g., a target isolate) or a biostimulant composition described herein may increase a crop yield of a plant treated by the microbe (e.g., a target isolate) or the biostimulant composition by at most about 75%, at most about 60%, at most about 50%, at most about 40%, at most about 35%, at most about 30%, at most about 25%, at most about 20%, at most about 18%, at most about 15%, at most about 12%, at most about 10%, at most about 5%, at most about 3%, at most about 2%, at most about 1.5%, at most about 1%, or at most about 0.5% compared to that of an untreated control plant. In some embodiments, a population of the microbial strain (e.g., a target isolate) or a biostimulant composition described herein may increase a crop yield of a plant treated by the microbe (e.g., a target isolate) or the biostimulant composition from about 0.1% to about 50% compared to that of an untreated control plant. In some embodiments, a population of the microbial strain (e.g., a target isolate) or a biostimulant composition described herein may increase a crop yield of a plant treated by the microbe (e.g., a target isolate) or the biostimulant composition from about 0.1% to about 0.2%, about 0.1% to about 0.5%, about 0.1% to about 1%, about 0.1% to about 2%, about 0.1% to about 3%, about 0.1% to about 5%, about 0.1% to about 10%, about 0.1% to about 15%, about 0.1% to about 20%, about 0.1% to about 25%, about 0.1% to about 50%, about 0.2% to about 0.5%, about 0.2% to about 1%, about 0.2% to about 2%, about 0.2% to about 3%, about 0.2% to about 5%, about 0.2% to about 10%, about 0.2% to about 15%, about 0.2% to about 20%, about 0.2% to about 25%, about 0.2% to about 50%, about 0.5% to about 1%, about 0.5% to about 2%, about 0.5% to about 3%, about 0.5% to about 5%, about 0.5% to about 10%, about 0.5% to about 15%, about 0.5% to about 20%, about 0.5% to about 25%, about 0.5% to about 50%, about 1% to about 2%, about 1% to about 3%, about 1% to about 5%, about 1% to about 10%, about 1% to about 15%, about 1% to about 20%, about 1% to about 25%, about 1% to about 50%, about 2% to about 3%, about 2% to about 5%, about 2% to about 10%, about 2% to about 15%, about 2% to about 20%, about 2% to about 25%, about 2% to about 50%, about 3% to about 5%, about 3% to about 10%, about 3% to about 15%, about 3% to about 20%, about 3% to about 25%, about 3% to about 50%, about 5% to about 10%, about 5% to about 15%, about 5% to about 20%, about 5% to about 25%, about 5% to about 50%, about 10% to about 15%, about 10% to about 20%, about 10% to about 25%, about 10% to about 50%, about 15% to about 20%, about 15% to about 25%, about 15% to about 50%, about 20% to about 25%, about 20% to about 50%, or about 25% to about 50% compared to that of an untreated control plant.

Average leaf area may be an important characteristic of plant growth, wherein a healthy plant may have a larger leave or larger leaves. In some embodiments, a larger leaf (e.g., greater average leaf area) may indicate better plant growth. In some embodiments, a smaller leaf (e.g., lower average leaf area) may indicate better plant growth. In some embodiments, a population of the microbial strain (e.g., a target isolate) or a biostimulant composition described herein may increase an average leaf area. A population of the microbial strain (e.g., a target isolate) or a biostimulant composition described herein may increase an average leaf area of a plant treated by the microbe (e.g., a target isolate) or the biostimulant composition by at least about 5%, at least about 10%, at least about 12%, at least about 15%, at least about 18%, at least about 20%, at least about 25%, at least about 30%, at least about 35%, at least about 40%, at least about 50%, at least about 60%, or at least about 75% compared to that of an untreated control plant. A population of the microbial strain (e.g., a target isolate) or a biostimulant composition described herein may increase an average leaf area of a plant treated by the microbe (e.g., a target isolate) or the biostimulant composition by at most about 75%, at most about 60%, at most about 50%, at most about 40%, at most about 35%, at most about 30%, at most about 25%, at most about 20%, at most about 18%, at most about 15%, at most about 12%, at most about 10%, or at most about 5% compared to that of an untreated control plant. A population of the microbial strain (e.g., a target isolate) or a biostimulant composition described herein may increase an average leaf area of a plant treated by the microbe (e.g., a target isolate) or the biostimulant composition from about 3% to about 75% compared to that of an untreated control plant. A population of the microbial strain (e.g., a target isolate) or a biostimulant composition described herein may increase an average leaf area of a plant treated by the microbe (e.g., a target isolate) or the biostimulant composition from about 3% to about 5%, about 3% to about 8%, about 3% to about 10%, about 3% to about 12%, about 3% to about 15%, about 3% to about 20%, about 3% to about 25%, about 3% to about 30%, about 3% to about 40%, about 3% to about 50%, about 3% to about 75%, about 5% to about 8%, about 5% to about 10%, about 5% to about 12%, about 5% to about 15%, about 5% to about 20%, about 5% to about 25%, about 5% to about 30%, about 5% to about 40%, about 5% to about 50%, about 5% to about 75%, about 8% to about 10%, about 8% to about 12%, about 8% to about 15%, about 8% to about 20%, about 8% to about 25%, about 8% to about 30%, about 8% to about 40%, about 8% to about 50%, about 8% to about 75%, about 10% to about 12%, about 10% to about 15%, about 10% to about 20%, about 10% to about 25%, about 10% to about 30%, about 10% to about 40%, about 10% to about 50%, about 10% to about 75%, about 12% to about 15%, about 12% to about 20%, about 12% to about 25%, about 12% to about 30%, about 12% to about 40%, about 12% to about 50%, about 12% to about 75%, about 15% to about 20%, about 15% to about 25%, about 15% to about 30%, about 15% to about 40%, about 15% to about 50%, about 15% to about 75%, about 20% to about 25%, about 20% to about 30%, about 20% to about 40%, about 20% to about 50%, about 20% to about 75%, about 25% to about 30%, about 25% to about 40%, about 25% to about 50%, about 25% to about 75%, about 30% to about 40%, about 30% to about 50%, about 30% to about 75%, about 40% to about 50%, about 40% to about 75%, or about 50% to about 75% compared to that of an untreated control plant.

A population of the microbial strain (e.g., a target isolate) or a biostimulant composition described herein may increase an average leaf area of a plant treated by the microbe (e.g., a target isolate) or the biostimulant composition from about 60% to about 250% compared to that of an untreated control plant. A population of the microbial strain (e.g., a target isolate) or a biostimulant composition described herein may increase an average leaf area of a plant treated by the microbe (e.g., a target isolate) or the biostimulant composition from about 60% to about 70%, about 60% to about 80%, about 60% to about 90%, about 60% to about 100%, about 60% to about 110%, about 60% to about 125%, about 60% to about 150%, about 60% to about 175%, about 60% to about 200%, about 60% to about 250%, about 70% to about 80%, about 70% to about 90%, about 70% to about 100%, about 70% to about 110%, about 70% to about 125%, about 70% to about 150%, about 70% to about 175%, about 70% to about 200%, about 70% to about 250%, about 80% to about 90%, about 80% to about 100%, about 80% to about 110%, about 80% to about 125%, about 80% to about 150%, about 80% to about 175%, about 80% to about 200%, about 80% to about 250%, about 90% to about 100%, about 90% to about 110%, about 90% to about 125%, about 90% to about 150%, about 90% to about 175%, about 90% to about 200%, about 90% to about 250%, about 100% to about 110%, about 100% to about 125%, about 100% to about 150%, about 100% to about 175%, about 100% to about 200%, about 100% to about 250%, about 110% to about 125%, about 110% to about 150%, about 110% to about 175%, about 110% to about 200%, about 110% to about 250%, about 125% to about 150%, about 125% to about 175%, about 125% to about 200%, about 125% to about 250%, about 150% to about 175%, about 150% to about 200%, about 150% to about 250%, about 175% to about 200%, about 175% to about 250%, or about 200% to about 250% compared to that of an untreated control plant.

Chlorophyll is an important photosynthetic pigment in plants. Management of chlorophyll content may help optimize light absorption, photosynthesis, and growth for a plant. In some embodiments, a population of a microbial strain as described herein (e.g., a target isolate) or a biostimulant composition described herein may increase chlorophyll content in a plant. In some embodiments, a population of the microbial strain (e.g., a target isolate) or a biostimulant composition described herein may increase chlorophyll content of a plant treated by the microbe (e.g., a target isolate) or the biostimulant composition by at least 1%, at least about 2%, at least about 3%, at least about 4%, at least about 5%, at least about 10%, at least about 12%, at least about 15%, at least about 18%, at least about 20%, at least about 25%, at least about 30%, at least about 35%, at least about 40%, or at least about 50% compared to that of an untreated control plant. In some embodiments, a population of the microbial strain (e.g., a target isolate) or a biostimulant composition described herein may increase chlorophyll content of a plant treated by the microbe (e.g., a target isolate) or the biostimulant composition by at most about 50%, at most about 40%, at most about 35%, at most about 30%, at most about 25%, at most about 20%, at most about 18%, at most about 15%, at most about 12%, at most about 10%, at most about 5%, at most about 4%, at most about 3%, at most about 2%, or at most about 1% compared to that of an untreated control plant. In some embodiments, a population of the microbial strain (e.g., a target isolate) or a biostimulant composition described herein may increase chlorophyll content of a plant treated by the microbe (e.g., a target isolate) or the biostimulant composition from about 0.5% to about 50% compared to that of an untreated control plant. In some embodiments, a population of the microbial strain (e.g., a target isolate) or a biostimulant composition described herein may increase chlorophyll content of a plant treated by the microbe (e.g., a target isolate) or the biostimulant composition from about 0.5% to about 1%, about 0.5% to about 2%, about 0.5% to about 3%, about 0.5% to about 4%, about 0.5% to about 5%, about 0.5% to about 8%, about 0.5% to about 10%, about 0.5% to about 15%, about 0.5% to about 20%, about 0.5% to about 25%, about 0.5% to about 50%, about 1% to about 2%, about 1% to about 3%, about 1% to about 4%, about 1% to about 5%, about 1% to about 8%, about 1% to about 10%, about 1% to about 15%, about 1% to about 20%, about 1% to about 25%, about 1% to about 50%, about 2% to about 3%, about 2% to about 4%, about 2% to about 5%, about 2% to about 8%, about 2% to about 10%, about 2% to about 15%, about 2% to about 20%, about 2% to about 25%, about 2% to about 50%, about 3% to about 4%, about 3% to about 5%, about 3% to about 8%, about 3% to about 10%, about 3% to about 15%, about 3% to about 20%, about 3% to about 25%, about 3% to about 50%, about 4% to about 5%, about 4% to about 8%, about 4% to about 10%, about 4% to about 15%, about 4% to about 20%, about 4% to about 25%, about 4% to about 50%, about 5% to about 8%, about 5% to about 10%, about 5% to about 15%, about 5% to about 20%, about 5% to about 25%, about 5% to about 50%, about 8% to about 10%, about 8% to about 15%, about 8% to about 20%, about 8% to about 25%, about 8% to about 50%, about 10% to about 15%, about 10% to about 20%, about 10% to about 25%, about 10% to about 50%, about 15% to about 20%, about 15% to about 25%, about 15% to about 50%, about 20% to about 25%, about 20% to about 50%, or about 25% to about 50% compared to that of an untreated control plant.

Chlorophyll content may be measured by a Soil Plant Analysis Development (SPAD) unit (e.g., an index value having a correlation to chlorophyll density). Thus, improved chlorophyll content may be quantified as an increase in SPAD units of a plant and can be an indication of improved plant health and/or growth. In some embodiments, a plant treated with a population of a microbial strain as described herein (e.g., a target isolate) or a biostimulant composition described herein may have a leaf chlorophyll content of at least about 22 SPAD units SPAD units, at least about 22.5 SPAD units, at least about 23 SPAD units, at least about 23.25 SPAD units, at least about 24 SPAD units, at least about 24.5 SPAD units, at least about 25 SPAD units, at least about 25.5 SPAD units, at least about 26 SPAD units, at least about 27 SPAD units, at least about 28 SPAD units, at least about 29 SPAD units, at least about 30, or greater than about 30 SPAD units. In some embodiments, a plant treated with a population of a microbial strain as described herein (e.g., a target isolate) or a biostimulant composition described herein may have a leaf chlorophyll content of at most about 30 SPAD units, at most about 29 SPAD units, at most about 28 SPAD units, at most about 27 SPAD units, at most about 26 SPAD units, at most about 25.5 SPAD units, at most about 25 SPAD units, at most about 24.5 SPAD units, at most about 24 SPAD units, at most about 23.5 SPAD units, at most about 23 SPAD units, at most about 22.5 SPAD units, at most about 22, or less than about 22 SPAD units. In some embodiments, a plant treated with a population of a microbial strain as described herein (e.g., a target isolate) or a biostimulant composition described herein may have a leaf chlorophyll content from about 21 SPAD units to about 35 SPAD units. In some embodiments, a plant treated with a population of a microbial strain as described herein (e.g., a target isolate) or a biostimulant composition described herein may have a leaf chlorophyll content from about 21 SPAD units to about 22 SPAD units, about 21 SPAD units to about 23 SPAD units, about 21 SPAD units to about 24 SPAD units, about 21 SPAD units to about 25 SPAD units, about 21 SPAD units to about 26 SPAD units, about 21 SPAD units to about 27 SPAD units, about 21 SPAD units to about 28 SPAD units, about 21 SPAD units to about 29 SPAD units, about 21 SPAD units to about 30 SPAD units, about 21 SPAD units to about 32 SPAD units, about 21 SPAD units to about 35 SPAD units, about 22 SPAD units to about 23 SPAD units, about 22 SPAD units to about 24 SPAD units, about 22 SPAD units to about 25 SPAD units, about 22 SPAD units to about 26 SPAD units, about 22 SPAD units to about 27 SPAD units, about 22 SPAD units to about 28 SPAD units, about 22 SPAD units to about 29 SPAD units, about 22 SPAD units to about 30 SPAD units, about 22 SPAD units to about 32 SPAD units, about 22 SPAD units to about 35 SPAD units, about 23 SPAD units to about 24 SPAD units, about 23 SPAD units to about 25 SPAD units, about 23 SPAD units to about 26 SPAD units, about 23 SPAD units to about 27 SPAD units, about 23 SPAD units to about 28 SPAD units, about 23 SPAD units to about 29 SPAD units, about 23 SPAD units to about 30 SPAD units, about 23 SPAD units to about 32 SPAD units, about 23 SPAD units to about 35 SPAD units, about 24 SPAD units to about 25 SPAD units, about 24 SPAD units to about 26 SPAD units, about 24 SPAD units to about 27 SPAD units, about 24 SPAD units to about 28 SPAD units, about 24 SPAD units to about 29 SPAD units, about 24 SPAD units to about 30 SPAD units, about 24 SPAD units to about 32 SPAD units, about 24 SPAD units to about 35 SPAD units, about 25 SPAD units to about 26 SPAD units, about 25 SPAD units to about 27 SPAD units, about 25 SPAD units to about 28 SPAD units, about 25 SPAD units to about 29 SPAD units, about 25 SPAD units to about 30 SPAD units, about 25 SPAD units to about 32 SPAD units, about 25 SPAD units to about 35 SPAD units, about 26 SPAD units to about 27 SPAD units, about 26 SPAD units to about 28 SPAD units, about 26 SPAD units to about 29 SPAD units, about 26 SPAD units to about 30 SPAD units, about 26 SPAD units to about 32 SPAD units, about 26 SPAD units to about 35 SPAD units, about 27 SPAD units to about 28 SPAD units, about 27 SPAD units to about 29 SPAD units, about 27 SPAD units to about 30 SPAD units, about 27 SPAD units to about 32 SPAD units, about 27 SPAD units to about 35 SPAD units, about 28 SPAD units to about 29 SPAD units, about 28 SPAD units to about 30 SPAD units, about 28 SPAD units to about 32 SPAD units, about 28 SPAD units to about 35 SPAD units, about 29 SPAD units to about 30 SPAD units, about 29 SPAD units to about 32 SPAD units, about 29 SPAD units to about 35 SPAD units, about 30 SPAD units to about 32 SPAD units, about 30 SPAD units to about 35 SPAD units, or about 32 SPAD units to about 35 SPAD units.

Application of the biostimulant composition may improve photosynthetic activity of a plant. Enhanced photosynthesis may result in more energy for the plant which may be metabolized and used to fuel the plant. Photosynthetic activity may be measured by a quantum yield and/or an electron transport rate of a plant. A quantum yield of photosynthesis can be a percentage of energetic efficiency of photoautotrophy. In some embodiments, a greater quantum yield may indicate more efficient photosynthetic activity and a healthier plant. In some embodiments, a plant treated with a population of a microbial strain as described herein (e.g., a target isolate) or a biostimulant composition described herein may have a quantum yield of at least about 45%, at least about 50%, at least about 55%, at least about 60%, at least about 61%, at least about 62%, at least about 63%, at least about 64%, at least about 65%, at least about 66%, at least about 67%, at least about 68%, at least about 69%, at least about 70%, at least about 75%, at least about 80%, or greater than about 80%. In some embodiments, a plant treated with a population of a microbial strain as described herein (e.g., a target isolate) or a biostimulant composition described herein may have a quantum yield of at most about 80%, at most about 75%, at most about 70%, at most about 69%, at most about 68%, at most about 67%, at most about 66%, at most about 65%, at most about 64%, at most about 63%, at most about 62%, at most about 61%, at most about 60%, at most about 55%, at most about 50%, at most about 45%, or less than about 45%. In some embodiments, a plant treated with a population of a microbial strain as described herein (e.g., a target isolate) or a biostimulant composition described herein may have a quantum yield from about 40% to about 80%. In some embodiments, a plant treated with a population of a microbial strain as described herein (e.g., a target isolate) or a biostimulant composition described herein may have a quantum yield from at most about 80%. In some embodiments, a plant treated with a population of a microbial strain as described herein (e.g., a target isolate) or a biostimulant composition described herein may have a quantum yield from about 40% to about 45%, about 40% to about 50%, about 40% to about 55%, about 40% to about 60%, about 40% to about 62%, about 40% to about 64%, about 40% to about 66%, about 40% to about 68%, about 40% to about 70%, about 40% to about 75%, about 40% to about 80%, about 45% to about 50%, about 45% to about 55%, about 45% to about 60%, about 45% to about 62%, about 45% to about 64%, about 45% to about 66%, about 45% to about 68%, about 45% to about 70%, about 45% to about 75%, about 45% to about 80%, about 50% to about 55%, about 50% to about 60%, about 50% to about 62%, about 50% to about 64%, about 50% to about 66%, about 50% to about 68%, about 50% to about 70%, about 50% to about 75%, about 50% to about 80%, about 55% to about 60%, about 55% to about 62%, about 55% to about 64%, about 55% to about 66%, about 55% to about 68%, about 55% to about 70%, about 55% to about 75%, about 55% to about 80%, about 60% to about 62%, about 60% to about 64%, about 60% to about 66%, about 60% to about 68%, about 60% to about 70%, about 60% to about 75%, about 60% to about 80%, about 62% to about 64%, about 62% to about 66%, about 62% to about 68%, about 62% to about 70%, about 62% to about 75%, about 62% to about 80%, about 64% to about 66%, about 64% to about 68%, about 64% to about 70%, about 64% to about 75%, about 64% to about 80%, about 66% to about 68%, about 66% to about 70%, about 66% to about 75%, about 66% to about 80%, about 68% to about 70%, about 68% to about 75%, about 68% to about 80%, about 70% to about 75%, about 70% to about 80%, or about 75% to about 80%.

The electronic transport chain of photosynthesis in plants couples electron transfer from sunlight-energized chlorophyll molecules through a series of membrane carriers resulting in the synthesis of ATP and NADPH. The respiratory electronic transport chain of plants couples electron transfer from organic substrates to oxygen molecules. Measures of an electron transport rate can quantify photosynthetic activity of a plant. In some embodiments, a greater electron transport rate may indicate more efficient photosynthetic activity and a healthier plant. In some embodiments, a plant treated with a population of a microbial strain as described herein (e.g., a target isolate) or a biostimulant composition described herein may have an electron transport rate of at least about 100 μmol m⁻²s⁻¹, at least about 110 μmol m⁻²s⁻¹, at least about 120 μmol m⁻²s⁻¹, at least about 130 μmol m⁻²s⁻¹, at least about 140 μmol m⁻²s⁻¹, at least about 150 μmol m⁻²s⁻¹, at least about 155 μmol m⁻²s⁻¹, at least about 160 μmol m⁻²s⁻¹, at least about 165 μmol m⁻²s⁻¹, at least about 170 μmol m⁻²s⁻¹, at least about 175 μmol m⁻²s⁻¹, at least about 180 μmol m⁻²s⁻¹, at least about 185 μmol m⁻²s⁻¹, at least about 190 μmol m⁻²s⁻¹, at least about 200 μmol m⁻²s⁻¹, or greater than about 200 μmol m⁻²s⁻¹. In some embodiments, a plant treated with a population of a microbial strain as described herein (e.g., a target isolate) or a biostimulant composition described herein may have an electron transport rate of at most about 200 μmol m⁻²s⁻¹, at most about 190 μmol m⁻²s⁻¹, at most about 185 μmol m⁻²s⁻¹, at most about 180 μmol m⁻²s⁻¹, at most about 175 μmol m⁻²s⁻¹, at most about 170 μmol m⁻²s⁻¹, at most about 165 μmol m⁻²s⁻¹, at most about 160 μmol m⁻²s⁻¹, at most about 155 μmol m⁻²s⁻¹, at most about 150 μmol m⁻²s⁻¹, at most about 140 μmol m⁻²s⁻¹, at most about 130 μmol m⁻²s⁻¹, at most about 120 μmol m⁻²s⁻¹, at most about 110 μmol m⁻²s⁻¹, at most about 100 μmol m⁻²s⁻¹, or less than about 100 μmol m⁻²s⁻¹. In some embodiments, a plant treated with a population of a microbial strain as described herein (e.g., a target isolate) or a biostimulant composition described herein may have an electron transport rate from about 100 μmol m⁻²s⁻¹ to about 200 μmol m⁻²s⁻¹. In some embodiments, a plant treated with a population of a microbial strain as described herein (e.g., a target isolate) or a biostimulant composition described herein may have an electron transport rate from about 100 μmol m⁻²s⁻¹ to about 110 μmol m⁻²s⁻¹, about 100 μmol m⁻²s⁻¹ to about 120 μmol m⁻²s⁻¹, about 100 μmol m⁻²s⁻¹ to about 130 μmol m⁻²s⁻¹, about 100 μmol m⁻²s⁻¹ to about 140 μmol m⁻²s⁻¹, about 100 μmol m⁻²s⁻¹ to about 150 μmol m⁻²s⁻¹, about 100 μmol m⁻²s⁻¹ to about 160 μmol m⁻²s⁻¹, about 100 μmol m⁻²s⁻¹ to about 170 μmol m⁻²s⁻¹, about 100 μmol m⁻²s⁻¹ to about 180 μmol m⁻²s⁻¹, about 100 μmol m⁻²s⁻¹ to about 190 μmol m⁻²s⁻¹, about 100 μmol m⁻²s⁻¹ to about 200 μmol m⁻²s⁻¹, about 110 μmol m⁻²s⁻¹ to about 120 μmol m⁻²s⁻¹, about 110 μmol m⁻²s⁻¹ to about 130 μmol m⁻²s⁻¹, about 110 μmol m⁻²s⁻¹ to about 140 μmol m⁻²s⁻¹, about 110 μmol m⁻²s⁻¹ to about 150 μmol m⁻²s⁻¹, about 110 μmol m⁻²s⁻¹ to about 160 μmol m⁻²s⁻¹, about 110 μmol m⁻²s⁻¹ to about 170 μmol m⁻²s⁻¹, about 110 μmol m⁻²s⁻¹ to about 180 μmol m⁻²s⁻¹, about 110 μmol m⁻²s⁻¹ to about 190 μmol m⁻²s⁻¹, about 110 μmol m⁻²s⁻¹ to about 200 μmol m⁻²s⁻¹, about 120 μmol m⁻²s⁻¹ to about 130 μmol m⁻²s⁻¹, about 120 μmol m⁻²s⁻¹ to about 140 μmol m⁻²s⁻¹, about 120 μmol m⁻²s⁻¹ to about 150 μmol m⁻²s⁻¹, about 120 μmol m⁻²s⁻¹ to about 160 μmol m⁻²s⁻¹, about 120 μmol m⁻²s⁻¹ to about 170 μmol m⁻²s⁻¹, about 120 μmol m⁻²s⁻¹ to about 180 μmol m⁻²s⁻¹, about 120 μmol m⁻²s⁻¹ to about 190 μmol m⁻²s⁻¹, about 120 μmol m⁻²s⁻¹ to about 200 μmol m⁻²s⁻¹, about 130 μmol m⁻²s⁻¹ to about 140 μmol m⁻²s⁻¹, about 130 μmol m⁻²s⁻¹ to about 150 μmol m⁻²s⁻¹, about 130 μmol m⁻²s⁻¹ to about 160 μmol m⁻²s⁻¹, about 130 μmol m⁻²s⁻¹ to about 170 μmol m⁻²s⁻¹, about 130 μmol m⁻²s⁻¹ to about 180 μmol m⁻²s⁻¹, about 130 μmol m⁻²s⁻¹ to about 190 μmol m⁻²s⁻¹, about 130 μmol m⁻²s⁻¹ to about 200 μmol m⁻²s⁻¹, about 140 μmol m⁻²s⁻¹ to about 150 μmol m⁻²s⁻¹, about 140 μmol m⁻²s⁻¹ to about 160 μmol m⁻²s⁻¹, about 140 μmol m⁻²s⁻¹ to about 170 μmol m⁻²s⁻¹, about 140 μmol m⁻²s⁻¹ to about 180 μmol m⁻²s⁻¹, about 140 μmol m⁻²s⁻¹ to about 190 μmol m⁻²s⁻¹, about 140 μmol m⁻²s⁻¹ to about 200 μmol m⁻²s⁻¹, about 150 μmol m⁻²s⁻¹ to about 160 μmol m⁻²s⁻¹, about 150 μmol m⁻²s⁻¹ to about 170 μmol m⁻²s⁻¹, about 150 μmol m⁻²s⁻¹ to about 180 μmol m⁻²s⁻¹, about 150 μmol m⁻²s⁻¹ to about 190 μmol m⁻²s⁻¹, about 150 μmol m⁻²s⁻¹ to about 200 μmol m⁻²s⁻¹, about 160 μmol m⁻²s⁻¹ to about 170 μmol m⁻²s⁻¹, about 160 μmol m⁻²s⁻¹ to about 180 μmol m⁻²s⁻¹, about 160 μmol m⁻²s⁻¹ to about 190 μmol m⁻²s⁻¹, about 160 μmol m⁻²s⁻¹ to about 200 μmol m⁻²s⁻¹, about 170 μmol m⁻²s⁻¹ to about 180 μmol m⁻²s⁻¹, about 170 μmol m⁻²s⁻¹ to about 190 μmol m⁻²s⁻¹, about 170 μmol m⁻²s⁻¹ to about 200 μmol m⁻²s⁻¹, about 180 μmol m⁻²s⁻¹ to about 190 μmol m⁻²s⁻¹, about 180 μmol m⁻²s⁻¹ to about 200 μmol m⁻²s⁻¹, or about 190 μmol m⁻²s⁻¹ to about 200 μmol m⁻²s⁻¹.

Plant height may be an important characteristic of plant growth, wherein a taller plant may indicate a healthier plant. In some embodiments, a taller plant may indicate better plant growth. In some embodiments, a shorter plant may indicate better plant growth. Plant height may be an important characteristic of plant growth, wherein a taller plant may indicate a healthier plant. In some embodiments, a taller plant may indicate better plant growth. In some embodiments, a shorter plant may indicate better plant growth. In some embodiments, a population of the microbial strain (e.g., a target isolate) or a biostimulant composition described herein may increase the height of a plant. In some embodiments, a population of the microbial strain (e.g., a target isolate) or a biostimulant composition described herein may increase a plant height of a plant treated by the microbe (e.g., a target isolate) or the biostimulant composition by at least 1%, at least about 2%, at least about 3%, at least about 4%, at least about 5%, at least about 10%, at least about 12%, at least about 15%, at least about 18%, at least about 20%, at least about 25%, at least about 30%, at least about 35%, at least about 40%, or at least about 50% compared to that of an untreated control plant. In some embodiments, a population of the microbial strain (e.g., a target isolate) or a biostimulant composition described herein may increase a plant height of a plant treated by the microbe (e.g., a target isolate) or the biostimulant composition by at most about 50%, at most about 40%, at most about 35%, at most about 30%, at most about 25%, at most about 20%, at most about 18%, at most about 15%, at most about 12%, at most about 10%, at most about 5%, at most about 4%, at most about 3%, at most about 2%, or at most about 1% compared to that of an untreated control plant. In some embodiments, a population of the microbial strain (e.g., a target isolate) or a biostimulant composition described herein may increase a plant height of a plant treated by the microbe (e.g., a target isolate) or the biostimulant composition from about 0.5% to about 50% compared to that of an untreated control plant. In some embodiments, a population of the microbial strain (e.g., a target isolate) or a biostimulant composition described herein may increase a plant height of a plant treated by the microbe (e.g., a target isolate) or the biostimulant composition from about 0.5% to about 1%, about 0.5% to about 2%, about 0.5% to about 3%, about 0.5% to about 4%, about 0.5% to about 5%, about 0.5% to about 8%, about 0.5% to about 10%, about 0.5% to about 15%, about 0.5% to about 20%, about 0.5% to about 25%, about 0.5% to about 50%, about 1% to about 2%, about 1% to about 3%, about 1% to about 4%, about 1% to about 5%, about 1% to about 8%, about 1% to about 10%, about 1% to about 15%, about 1% to about 20%, about 1% to about 25%, about 1% to about 50%, about 2% to about 3%, about 2% to about 4%, about 2% to about 5%, about 2% to about 8%, about 2% to about 10%, about 2% to about 15%, about 2% to about 20%, about 2% to about 25%, about 2% to about 50%, about 3% to about 4%, about 3% to about 5%, about 3% to about 8%, about 3% to about 10%, about 3% to about 15%, about 3% to about 20%, about 3% to about 25%, about 3% to about 50%, about 4% to about 5%, about 4% to about 8%, about 4% to about 10%, about 4% to about 15%, about 4% to about 20%, about 4% to about 25%, about 4% to about 50%, about 5% to about 8%, about 5% to about 10%, about 5% to about 15%, about 5% to about 20%, about 5% to about 25%, about 5% to about 50%, about 8% to about 10%, about 8% to about 15%, about 8% to about 20%, about 8% to about 25%, about 8% to about 50%, about 10% to about 15%, about 10% to about 20%, about 10% to about 25%, about 10% to about 50%, about 15% to about 20%, about 15% to about 25%, about 15% to about 50%, about 20% to about 25%, about 20% to about 50%, or about 25% to about 50% compared to that of an untreated control plant.

### VII. Microbial Digestion Methods and Systems

### A. System Overview

Certain embodiments disclosed herein comprise methods and systems in which microbes comprised in microbial consortia digest substances provided in a feedstock. The bioreactor systems may be comprised of a series of separate, fluidly connected containers, also referred to herein as “reactors.” In each reactor, a different microbial consortium may be established and maintained throughout continuous operation of the bioreactor system. The unique microbial consortia present in each reactor may provide for different physiological activities in the different reactors. Thus, different steps in digestion of a feedstock may be performed in different reactors, which may result in (1) a more complete digestion—i.e., a more complete breakdown of macromolecules in the feedstock—than other types of bioreactor systems, and/or (2) production of a variety of microbial digestion products having plant growth promoting properties In some embodiments, conditions that may allow for an enrichment of a microbial community with a targeted functionality (e.g., zinc solubilization, phosphate solubilization, nitrogen use efficiency) may comprise a retention time, addition of a selective pressure, hydraulic flow rate, one or more carbon sources, one or more nitrogen sources, pH, nutrient mix, microaerobic conditions, recirculation of a working fluid, or any combination thereof.

In some embodiments, a reactor or a series of reactors functions to contribute to the growth of one or more microbes having desired plant growth promoting properties and/or to the production of digestion products having plant growth promoting properties. In some embodiments, the bioreactor system (e.g., the bioreactor system) comprises an established population of one or more microbial strains (e.g., nutrient solubilizing microbial strain) in one or more containers of the system. An “established population” of a particular microbial strain is a population that remains within an operating bioreactor system without replenishing the microbial strain from outside the bioreactor system. For example, an established population of a particular microbial strain can be a population of a microbial strain that remains in one or more containers of a bioreactor system without re-inoculation of the microbial strain. In some embodiments, an established population of the microbial strain is a population of microbial strain that has not been diminished in concentration by more than about 1%, more than about 2%, more than about 3%, more than about 4%, more than about 5%, more than about 6%, more than about 7%, more than about 8%, more than about 9%, more than about 10%, more than about 11%, more than about 12%, more than about 13%, more than about 14%, more than about 15%, more than about 16%, more than about 17%, more than about 18%, more than about 19%, more than about 20%, more than about 21%, more than about 22%, more than about 23%, more than about 24%, or more than about 25% during continuous operation of the bioreactor system for a period of time. In some embodiments, an established population is one that has not been diminished in concentration by less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, or less than about 1% during continuous operation of the bioreactor system for a period of time. In some embodiments, an established population is one that has not been diminished in concentration by about 1% to about 25%, about 2% to about 25%, about 3% to about 25%, about 4% to about 25%, about 5% to about 25%, about 6% to about 25%, about 7% to about 25%, about 8% to about 25%, about 9% to about 25%, about 10% to about 25%, about 15% to about 25%, about 20% to about 25%, about 1% to about 5%, about 1% to about 10%, about 1% to about 15%, about 1% to about 20%, about 5% to about 10%, about 5% to about 15%, about 5% to about 20%, about 10% to about 15%, about 10% to about 20%, or about 15% to about 20% during continuous operation of the bioreactor system for a period of time. The duration of time may be at least about 5 days, at least about 10 days, at least about 15 days, at least about 20 days, at least about 25 days, at least about 30 days, at least about 60 days, or at least about 90 days without adding a population of the microbial strain to the bioreactor system at a concentration amount. The duration of time may be at most about 90 days, at most about 60 days, at most about 30 days, at most about 25 days, at most about 20 days, at most about 15 days, at most about 10 days, or at most about 5 days without adding a population of the microbial strain to the bioreactor system at a concentration amount. The duration of time may be between about 5 days to about 10 days, about 5 days to about 15 days, about 5 days to about 20 days, about 5 days to about 25 days, about 5 days to about 30 days, about 5 days to about 60 days, about 5 days to about 90 days, about 10 days to about 15 days, about 10 days to about 20 days, about 10 days to about 25 days, about 10 days to about 30 days, about 10 days to about 60 days, about 10 days to about 90 days, about 15 days to about 20 days, about 15 days to about 25 days, about 15 days to about 30 days, about 15 days to about 60 days, about 15 days to about 90 days, about 20 days to about 25 days, about 20 days to about 30 days, about 20 days to about 60 days, about 20 days to about 90 days, about 30 days to about 60 days, about 30 days to about 90 days, about 60 days to about 90 days without adding a population of the microbial strain to the bioreactor system at a concentration amount. The concentration of the microbial strain population in the bioreactor system may be greater than about 1 CFU/ml, greater than about 2 CFU/ml, greater than about 3 CFU/ml, greater than about 4 CFU/ml, greater than about 5 CFU/ml, greater than about 6 CFU/ml, greater than about 7 CFU/ml, greater than about 8 CFU/ml, greater than about 9 CFU/ml, greater than about 10 CFU/ml, greater than about 11 CFU/ml, greater than about 12 CFU/ml, greater than about 13 CFU/ml, greater than about 14 CFU/ml, greater than about 15 CFU/ml, greater than about 25 CFU/ml, greater than about 50 CFU/ml, or greater than about 100 CFU/ml. The concentration of the microbial strain population in the bioreactor system may be less than about 100 CFU/ml, less than about 50 CFU/ml, less than about 25 CFU/ml, less than about 15 CFU/ml, less than about 14 CFU/ml, less than about 13 CFU/ml, less than about 12 CFU/ml, less than about 11 CFU/ml, less than about 10 CFU/ml, less than about 9 CFU/ml, less than about 8 CFU/ml, less than about 7 CFU/ml, less than about 6 CFU/ml, less than about 5 CFU/ml, less than about 4 CFU/ml, less than about 3 CFU/ml, less than about 2 CFU/ml, or less than about 1 CFU/ml. The concentration of the microbial strain population in the bioreactor system may be about 1 CFU/ml to about 2 CFU/ml, about 1 CFU/ml to about 3 CFU/ml, about 1 CFU/ml to about 4 CFU/ml, about 1 CFU/ml to about 5 CFU/ml, about 1 CFU/ml to about 6 CFU/ml, about 1 CFU/ml to about 7 CFU/ml, about 1 CFU/ml to about 8 CFU/ml, about 1 CFU/ml to about 9 CFU/ml, about 1 CFU/ml to about 10 CFU/ml, about 1 CFU/ml to about 11 CFU/ml, about 1 CFU/ml to about 12 CFU/ml, about 1 CFU/ml to about 13 CFU/ml, about 1 CFU/ml to about 14 CFU/ml, about 1 CFU/ml to about 15 CFU/ml, about 1 CFU/ml to about 25 CFU/ml, about 1 CFU/ml to about 50 CFU/ml, about 1 CFU/ml to about 100 CFU/ml, about 5 CFU/ml to about 10 CFU/ml, about 1 CFU/ml to about 15 CFU/ml, about 5 CFU/ml to about 25 CFU/ml, about 5 CFU/ml to about 50 CFU/ml, about 5 CFU/ml to about 100 CFU/ml, about 10 CFU/ml to about 15 CFU/ml, about 10 CFU/ml to about 25 CFU/ml, about 10 CFU/ml to about 50 CFU/ml, about 10 CFU/ml to about 100 CFU/ml, about 15 CFU/ml to about 25 CFU/ml, about 15 CFU/ml to about 50 CFU/ml, about 15 CFU/ml to about 100 CFU/ml, about 25 CFU/ml to about 50 CFU/ml, about 25 CFU/ml to about 100 CFU/ml, about 50 CFU/ml to about 100 CFU/ml. A concentration of the microbial strain may be measured using a method including, but not limited to, spectrophotometry, fluorescent or bioluminescent methods, or serial dilution.

In some embodiments, an established population of a microbial strain has been established by adding one or more inoculations of the microbial strain into one or more containers of the bioreactor system. In some embodiments, a measurement (e.g., a concentration or bacterial count) of an established population may be performed for a bioreactor system. In some embodiments, an established population is a population that is derived from a microbial population that was inoculated into the system at least about 5 days, at least about 10 days, at least about 20 days, at least about 30 days, at least about 40 days, at least about 50 days, at least about 60 days, or at least about 90 days prior to a measurement of the established population. In some embodiments, an established population is a population that is derived from a microbial population that was inoculated into the system at most about 90 days, at most about 60 days, at most about 50 days, at most about 40 days, at most about 30 days, at most about 20 days, at most about 10 days, or at most about 5 days prior to a measurement of the established population. In some embodiments, an established population is a population that is derived from a microbial population that was inoculated into the system about 5 days to about 10 days, about 5 days to about 20 days, about 5 days to about 30 days, about 5 days to about 40 days, about 5 days to about 50 days, about 5 days to about 60 days, about 5 days to about 90 days, about 10 days to about 20 days, about 10 days to about 30 days, about 10 days to about 40 days, about 10 days to about 50 days, about 10 days to about 60 days, about 10 days to about 90 days, about 20 days to about 30 days, about 20 days to about 40 days, about 20 days to about 50 days, about 20 days to about 60 days, about 20 days to about 90 days, about 30 days to about 40 days, about 30 days to about 50 days, about 30 days to about 60 days, about 30 days to about 90 days, about 40 days to about 50 days, about 40 days to about 60 days, about 40 days to about 90 days, about 50 days to about 60 days, about 50 days to about 90 days, about 60 days to about 90 days prior to a measurement of the established population.

A measurement (e.g., a concentration or bacterial count) may be taken at any point during system operation. For example, a measurement of an established population of a microbial strain may occur at the start of a bioreactor system operation (e.g., within a first minute, hour, or day of operation). Two or more measurements (e.g., concentrations or bacterial counts) may be made to examine a change in concentration or bacterial count of an established population of a microbial strain over time. As an example, a measurement of an established population of a microbial strain may be made at a start of operation and may then be made at least 1, 2, 3, 4, 5, 6, 12, 18, 24, 48, 72, or more hours later to provide a result of how the concentration or bacterial count of the established population of the microbial strain changed over time.

In some embodiments, a microbial inoculum comprises a single isolated microbe. An isolated microbe may comprise a microbe generated outside of a natural environment (e.g., by plating methods or culture mediums). In some embodiments, the microbial inoculum may comprise between 1 and 5 isolated microbes (e.g., microbial strains). In some embodiments, the inoculum may comprise 1, 2, 3, 4 or 5 isolated microbes (e.g., microbial strains). In some embodiments, the inoculum may comprise greater than 5 isolated microbes (e.g., microbial strains). For example, an isolated microbe may comprise a microbial strain separated from a natural and/or mixed population of microbes. In some embodiments, in addition to the one or more isolated microbes, a microbial inoculum input into a bioreactor system may comprise a complex mixture of microbes, which may comprise at least about 5, at least about 10, at least about 20, at least about 25, at least about 50, at least about 100, at least about 200, at least about 225, at least about 250, at least about 275, at least about 300, at least about 350, at least about 400, or more species of microbes. In some embodiments, an isolated microbe can be the same species as a microbe in the complex mixture of microbes. In some embodiments, an isolated microbe can be a different species as a microbe in the complex mixture of microbes. In some embodiments, in addition to the one or more isolated microbes, a microbial inoculum input into a bioreactor system may comprise a complex mixture of microbes, which may comprise at most about 400, at most about 350, at most about 300, at most about 275, at most about 250, at most about 225, at most about 200, at most about 100, at most about 50, at most about 25, at most about 20, at most about 10, at most about 5, or fewer species of microbes. In some embodiments, in addition to the one or more isolated microbes, a microbial inoculum input into a bioreactor system may comprise a complex mixture of microbes, which may comprise about 5 to about 10, about 5 to about 20, about 5 to about 25, about 5 to about 50, about 5 to about 100, about 5 to about 200, about 5 to about 250, about 5 to about 300, about 5 to about 350, about 10 to about 20, about 10 to about 25, about 10 to about 50, about 10 to about 100, about 10 to about 200, about 10 to about 250, about 10 to about 300, about 10 to about 350, about 10 to about 400, about 50 to about 100, about 50 to about 200, about 50 to about 150, about 50 to about 200, about 50 to about 250, about 50 to about 300, about 50 to about 350, about 50 to about 400, about 100 to about 150, about 100 to about 200, about 100 to about 250, about 100 to about 300, about 100 to about 350, about 100 to about 400, about 200 to about 250, about 200 to about 300, about 200 to about 350, about 200 to about 400, about 300 to about 400 species of microbes.

An inoculum of a microbe as described herein may have at least one plant growth promotion property (e.g., a property of plant growth). A plant growth promotion property may comprise shoot biomass, root biomass, nutrient uptake, crop yield, leaf area, chlorophyll content, increased photosynthetic activity, heat tolerance, cold tolerance, drought tolerance, salt tolerance, zinc uptake, zinc solubilization, phosphate uptake, phosphate solubilization, micronutrient uptake, or total biomass. A bioreactor system may be configured to enhance production of the inoculum of the microbe. A microbe may be a bacterial species, a fungal species, or an algal species.

In some embodiments, the inoculum of a microbe may comprise at least two isolated microbes. In some embodiments, the inoculum of a microbe may comprise at least one isolated microbe and at least one non-isolated microbe (e.g., a microbe naturally present in a feedstock material). In some embodiments, the inoculum of a microbe may comprise 1 microbe, 2 microbes, 3 microbes, 4 microbe, 5 microbes, or more microbes. An inoculum of a microbe may be provided and/or transferred to a first container (e.g., a reactor) of a bioreactor system one time, two times, three times, four times, five times, or more times. An inoculum of a microbe may be provided and/or transferred to a second container, a third container, a fourth container, a fifth container, a sixth container, or any container of a system described herein.

An inoculum of a microbe may have a concentration of at least about 1.0×10² CFU/ml, at least about 1.0×10³ CFU/ml, at least about 1.0×10⁴ CFU/ml, at least about 1.0×10⁵ CFU/ml, at least about 1.0×10⁶ CFU/ml, at least about 1.0×10⁷ CFU/ml, at least about 1.0×10⁸ CFU/ml, at least about 1.0×10⁹ CFU/ml, at least about 1.0×10¹⁰ CFU/ml, at least about 1.0×10¹¹ CFU/ml, or at least about 1.0×10¹² CFU/ml prior to transferring to a first container of a bioreactor system. An inoculum of a microbe may have a concentration of at most about 1.0×10² CFU/ml, at most about 1.0×10³ CFU/ml, at most about 1.0×10⁴ CFU/ml, at most about 1.0×10⁵ CFU/ml, at most about 1.0×10⁶ CFU/ml, at most about 1.0×10⁷ CFU/ml, at most about 1.0×10⁸ CFU/ml, at most about 1.0×10⁹ CFU/ml, at most about 1.0×10¹⁰ CFU/ml, at most about 1.0×10¹¹ CFU/ml, or at most about 1.0×10¹² CFU/ml prior to transferring to a first container of a bioreactor system. An inoculum of a microbe may have a concentration of about 1.0×10² CFU/ml to about 1.0×10³ CFU/ml, about 1.0×10² CFU/ml to about 1.0×10⁴ CFU/ml, about 1.0×10² CFU/ml to about 1.0×10⁵ CFU/ml, about 1.0×10² CFU/ml to about 1.0×10⁶ CFU/ml, about 1.0×10² CFU/ml to about 1.0×10⁷ CFU/ml, about 1.0×10² CFU/ml to about 1.0×10⁸ CFU/ml, about 1.0×10² CFU/ml to about 1.0×10⁹ CFU/ml, about 1.0×10² CFU/ml to about 1.0×10¹⁰ CFU/ml, about 1.0×10² CFU/ml to about 1.0×10¹¹ CFU/ml, about 1.0×10² CFU/ml to about 1.0×10¹² CFU/ml, 1.0×10³ CFU/ml to about 1.0×10⁴ CFU/ml, about 1.0×10³ CFU/ml to about 1.0×10⁵ CFU/ml, about 1.0×10³ CFU/ml to about 1.0×10⁶ CFU/ml, about 1.0×10³ CFU/ml to about 1.0×10⁷ CFU/ml, about 1.0×10³ CFU/ml to about 1.0×10⁸ CFU/ml, about 1.0×10³ CFU/ml to about 1.0×10⁹ CFU/ml, about 1.0×10³ CFU/ml to about 1.0×10¹⁰ CFU/ml, about 1.0×10³ CFU/ml to about 1.0×10¹¹ CFU/ml, about 1.0×10³ CFU/ml to about 1.0×10¹² CFU/ml, about 1.0×10⁴ CFU/ml to about 1.0×10⁵ CFU/ml, about 1.0×10⁴ CFU/ml to about 1.0×10⁶ CFU/ml, about 1.0×10⁴ CFU/ml to about 1.0×10⁷ CFU/ml, about 1.0×10⁴ CFU/ml to about 1.0×10⁸ CFU/ml, about 1.0×10⁴ CFU/ml to about 1.0×10⁹ CFU/ml, about 1.0×10⁴ CFU/ml to about 1.0×10¹⁰ CFU/ml, about 1.0×10⁴ CFU/ml to about 1.0×10¹¹ CFU/ml, about 1.0×10⁴ CFU/ml to about 1.0×10¹² CFU/ml, about 1.0×10⁵ CFU/ml to about 1.0×10⁶ CFU/ml, about 1.0×10⁵ CFU/ml to about 1.0×10⁷ CFU/ml, about 1.0×10⁵ CFU/ml to about 1.0×10⁸ CFU/ml, about 1.0×10⁵ CFU/ml to about 1.0×10⁹ CFU/ml, about 1.0×10⁵ CFU/ml to about 1.0×10¹⁰ CFU/ml, about 1.0×10⁵ CFU/ml to about 1.0×10¹¹ CFU/ml, about 1.0×10⁵ CFU/ml to about 1.0×10¹² CFU/ml, about 1.0×10⁶ CFU/ml to about 1.0×10⁷ CFU/ml, about 1.0×10⁶ CFU/ml to about 1.0×10⁸ CFU/ml, about 1.0×10⁶ CFU/ml to about 1.0×10⁹ CFU/ml, about 1.0×10⁶ CFU/ml to about 1.0×10¹⁰ CFU/ml, about 1.0×10⁶ CFU/ml to about 1.0×10¹¹ CFU/ml, about 1.0×10⁶ CFU/ml to about 1.0×10¹² CFU/ml about 1.0×10⁷ CFU/ml to about 1.0×10⁸ CFU/ml, about 1.0×10⁷ CFU/ml to about 1.0×10⁹ CFU/ml, about 1.0×10⁷ CFU/ml to about 1.0×10¹⁰ CFU/ml, about 1.0×10⁷ CFU/ml to about 1.0×10¹¹ CFU/ml, about 1.0×10⁷ CFU/ml to about 1.0×10¹² CFU/ml about 1.0×10⁸ CFU/ml to about 1.0×10⁹ CFU/ml, about 1.0×10⁸ CFU/ml to about 1.0×10¹⁰ CFU/ml, about 1.0×10⁸ CFU/ml to about 1.0×10¹¹ CFU/ml, about 1.0×10⁸ CFU/ml to about 1.0×10¹² CFU/ml, about 1.0×10⁹ CFU/ml to about 1.0×10¹⁰ CFU/ml, about 1.0×10⁹ CFU/ml to about 1.0×10¹¹ CFU/ml, about 1.0×10⁹ CFU/ml to about 1.0×10¹² CFU/ml, about 1.0×10¹⁰ CFU/ml to about 1.0×10¹¹ CFU/ml, about 1.0×10¹⁰ CFU/ml to about 1.0×10¹² CFU/ml, or about 1.0×10¹¹ CFU/ml to about 1.0×10¹² CFU/ml prior to transferring to a first container of a bioreactor system.

An inoculum of a microbe may have a concentration of at least about 1.0×10² CFU/ml, at least about 1.0×10³ CFU/ml, at least about 1.0×10⁴ CFU/ml, at least about 1.0×10⁵ CFU/ml, at least about 1.0×10⁶ CFU/ml, at least about 1.0×10⁷ CFU/ml, at least about 1.0×10⁸ CFU/ml, at least about 1.0×10⁹ CFU/ml, at least about 1.0×10¹⁰ CFU/ml, at least about 1.0×10¹¹ CFU/ml, or at least about 1.0×10¹² CFU/ml after transferring to a first container of a bioreactor system (e.g., a concentration of the microbe within the first container of the bioreactor system). An inoculum of a microbe may have a concentration of at most about 1.0×10² CFU/ml, at most about 1.0×10³ CFU/ml, at most about 1.0×10⁴ CFU/ml, at most about 1.0×10⁵ CFU/ml, at most about 1.0×10⁶ CFU/ml, at most about 1.0×10⁷ CFU/ml, at most about 1.0×10⁸ CFU/ml, at most about 1.0×10⁹ CFU/ml, at most about 1.0×10¹⁰ CFU/ml, at most about 1.0×10¹¹ CFU/ml, or at most about 1.0×10¹² CFU/ml after transferring to a first container of a bioreactor system. An inoculum of a microbe may have a concentration of about 1.0×10² CFU/ml to about 1.0×10³ CFU/ml, about 1.0×10² CFU/ml to about 1.0×10⁴ CFU/ml, about 1.0×10² CFU/ml to about 1.0×10⁵ CFU/ml, about 1.0×10² CFU/ml to about 1.0×10⁶ CFU/ml, about 1.0×10² CFU/ml to about 1.0×10⁷ CFU/ml, about 1.0×10² CFU/ml to about 1.0×10⁸ CFU/ml, about 1.0×10² CFU/ml to about 1.0×10⁹ CFU/ml, about 1.0×10² CFU/ml to about 1.0×10¹⁰ CFU/ml, about 1.0×10² CFU/ml to about 1.0×10¹¹ CFU/ml, about 1.0×10² CFU/ml to about 1.0×10¹² CFU/ml, 1.0×10³ CFU/ml to about 1.0×10⁴ CFU/ml, about 1.0×10³ CFU/ml to about 1.0×10⁵ CFU/ml, about 1.0×10³ CFU/ml to about 1.0×10⁶ CFU/ml, about 1.0×10³ CFU/ml to about 1.0×10⁷ CFU/ml, about 1.0×10³ CFU/ml to about 1.0×10⁸ CFU/ml, about 1.0×10³ CFU/ml to about 1.0×10⁹ CFU/ml, about 1.0×10³ CFU/ml to about 1.0×10¹⁰ CFU/ml, about 1.0×10³ CFU/ml to about 1.0×10¹¹ CFU/ml, about 1.0×10³ CFU/ml to about 1.0×10¹² CFU/ml, about 1.0×10⁴ CFU/ml to about 1.0×10⁵ CFU/ml, about 1.0×10⁴ CFU/ml to about 1.0×10⁶ CFU/ml, about 1.0×10⁴ CFU/ml to about 1.0×10⁷ CFU/ml, about 1.0×10⁴ CFU/ml to about 1.0×10⁸ CFU/ml, about 1.0×10⁴ CFU/ml to about 1.0×10⁹ CFU/ml, about 1.0×10⁴ CFU/ml to about 1.0×10¹⁰ CFU/ml, about 1.0×10⁴ CFU/ml to about 1.0×10¹¹ CFU/ml, about 1.0×10⁴ CFU/ml to about 1.0×10¹² CFU/ml, about 1.0×10⁵ CFU/ml to about 1.0×10⁶ CFU/ml, about 1.0×10⁵ CFU/ml to about 1.0×10⁷ CFU/ml, about 1.0×10⁵ CFU/ml to about 1.0×10⁸ CFU/ml, about 1.0×10⁵ CFU/ml to about 1.0×10⁹ CFU/ml, about 1.0×10⁵ CFU/ml to about 1.0×10¹⁰ CFU/ml, about 1.0×10⁵ CFU/ml to about 1.0×10¹¹ CFU/ml, about 1.0×10⁵ CFU/ml to about 1.0×10¹² CFU/ml, about 1.0×10⁶ CFU/ml to about 1.0×10⁷ CFU/ml, about 1.0×10⁶ CFU/ml to about 1.0×10⁸ CFU/ml, about 1.0×10⁶ CFU/ml to about 1.0×10⁹ CFU/ml, about 1.0×10⁶ CFU/ml to about 1.0×10¹⁰ CFU/ml, about 1.0×10⁶ CFU/ml to about 1.0×10¹¹ CFU/ml, about 1.0×10⁶ CFU/ml to about 1.0×10¹² CFU/ml about 1.0×10⁷ CFU/ml to about 1.0×10⁸ CFU/ml, about 1.0×10⁷ CFU/ml to about 1.0×10⁹ CFU/ml, about 1.0×10⁷ CFU/ml to about 1.0×10¹⁰ CFU/ml, about 1.0×10⁷ CFU/ml to about 1.0×10¹¹ CFU/ml, about 1.0×10⁷ CFU/ml to about 1.0×10¹² CFU/ml about 1.0×10⁸ CFU/ml to about 1.0×10⁹ CFU/ml, about 1.0×10⁸ CFU/ml to about 1.0×10¹⁰ CFU/ml, about 1.0×10⁸ CFU/ml to about 1.0×10¹¹ CFU/ml, about 1.0×10⁸ CFU/ml to about 1.0×10¹² CFU/ml, about 1.0×10⁹ CFU/ml to about 1.0×10¹⁰ CFU/ml, about 1.0×10⁹ CFU/ml to about 1.0×10¹¹ CFU/ml, about 1.0×10⁹ CFU/ml to about 1.0×10¹² CFU/ml, about 1.0×10¹⁰ CFU/ml to about 1.0×10¹¹ CFU/ml, about 1.0×10¹⁰ CFU/ml to about 1.0×10¹² CFU/ml, or about 1.0×10¹¹ CFU/ml to about 1.0×10¹² CFU/ml after transferring to a first container of a bioreactor system.

In some embodiments, the inoculum comprises a single microbe. In some embodiments, the inoculum comprises a single microbial strain. The terms “microbe” and “microbial strain” can be used interchangeably throughout. In some embodiments, the inoculum may comprise between 1 and 5 microbes. In some embodiments, the inoculum may comprise at least 1, at least 2, at least 3, at least 4, at least 5, or more microbe(s). In some embodiments, the inoculum may comprise at most 5, at most 4, at most 3, at most 2, or at most 1 microbe(s).

In some embodiments, a bioreactor system (e.g., one or more containers of a bioreactor system) comprises at least one microbial strain. In some embodiments, a bioreactor system (e.g., one or more containers of a bioreactor system) comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more microbial strains. In some embodiments, a bioreactor system (e.g., one or more containers of a bioreactor system) comprises at least one nutrient-solubilizing microbial strain (e.g., a zinc-solubilizing microbial strain, a phosphate-solubilizing microbial strain, an ammonia-solubilizing strain). In some embodiments, a bioreactor system (e.g., one or more containers of a bioreactor system) comprises an established population of a first microbial strain and an established population of a second microbial strain. In some embodiments, the bioreactor system further comprises an established population of a third microbial strain. In some embodiments, the microbial strains can have similar or substantially similar biostimulant properties (e.g., two or more strains are each zinc-solubilizing microbial strains, two or more strains are nitrogen-fixing strains, two or more strains are phosphate-solubilizing microbial strains). In some embodiments, the microbial strains have different biostimulant properties. The established populations of the respective microbial strains may be established in individual or combined inoculations into the bioreactor system. An individual inoculation may comprise one inoculum of a microbial strain (e.g., microbe). A combined inoculation may comprise an inoculum comprising at least two microbial strains. The combined inoculation may comprise the same isolated microbial strains. The combined inoculation may comprise an isolated microbial strain and non-isolated microbial strain. The combined inoculation may comprise two or more isolated microbial strains.

In some embodiments, a bioreactor system (e.g., one or more containers of a bioreactor system) comprises an established population of a zinc-solubilizing microbial strains. In some embodiments, a bioreactor system (e.g., one or more containers of a bioreactor system) comprises an established population of a phosphate-solubilizing microbial strain. In some embodiments, a bioreactor system (e.g., one or more containers of a bioreactor system) comprises an established population of an ammonia-solubilizing strain, a nitrogen use efficiency microbial strain, a nitrogen fixing microbial strain, or any combination thereof.

In some embodiments, a bioreactor system (e.g., one or more containers of a bioreactor system) comprises an established population of a first microbial strain (e.g., a zinc-solubilizing microbial strain, a phosphate-solubilizing microbial strain, an ammonia-solubilizing strain, a nitrogen use efficiency microbial strain, or a nitrogen fixating microbial strain) and further comprises a second established population of a zinc-solubilizing microbial strain. In some embodiments, a bioreactor system (e.g., one or more containers of a bioreactor system) comprises an established population of a first microbial strain (e.g., a zinc-solubilizing microbial strain, a phosphate-solubilizing microbial strain, an ammonia-solubilizing strain, a nitrogen use efficiency microbial strain, or a nitrogen fixating microbial strain) and further comprises a second established population of a phosphate-solubilizing microbial strain. In some embodiments, a bioreactor system (e.g., one or more containers of a bioreactor system) comprises an established population of a first microbial strain (e.g., a zinc-solubilizing microbial strain, a phosphate-solubilizing microbial strain, an ammonia-solubilizing strain, a nitrogen use efficiency microbial strain, or a nitrogen fixating microbial strain) and further comprises a second established population of an ammonia-solubilizing strain, a nitrogen use efficiency microbial strain, or a nitrogen fixating microbial strain.

In some embodiments, a bioreactor system (e.g., one or more containers of a bioreactor system) comprises an established population of a first microbial strain (e.g., a zinc-solubilizing microbial strain, a phosphate-solubilizing microbial strain, an ammonia-forming strain, a nitrogen use efficiency microbial strain, or a nitrogen fixating microbial strain) and a second microbial strain (e.g., a zinc-solubilizing microbial strain, a phosphate-solubilizing microbial strain, an ammonia-forming strain, a nitrogen use efficiency microbial strain, or a nitrogen fixating microbial strain) and further comprises a third established population of a zinc-solubilizing microbial strain. In some embodiments, a bioreactor system (e.g., one or more containers of a bioreactor system) comprises an established population of a first microbial strain (e.g., a zinc-solubilizing microbial strain, a phosphate-solubilizing microbial strain, an ammonia-forming strain, a nitrogen use efficiency microbial strain, or a nitrogen fixating microbial strain) and a second microbial strain (e.g., a zinc-solubilizing microbial strain, a phosphate-solubilizing microbial strain, an ammonia-forming strain, a nitrogen use efficiency microbial strain, or a nitrogen fixating microbial strain) and further comprises a third established population of a phosphate-solubilizing microbial strain. In some embodiments, a bioreactor system (e.g., one or more containers of a bioreactor system) comprises an established population of a first microbial strain (e.g., a zinc-solubilizing microbial strain, a phosphate-solubilizing microbial strain, an ammonia-forming strain, a nitrogen use efficiency microbial strain, or a nitrogen fixating microbial strain) and a second microbial strain (e.g., a zinc-solubilizing microbial strain, a phosphate-solubilizing microbial strain, an ammonia-forming strain, a nitrogen use efficiency microbial strain, or a nitrogen fixating microbial strain) and further comprises a third established population of an ammonia-forming strain, a nitrogen use efficiency microbial strain, or a nitrogen fixating microbial strain.

In some embodiments, the feedstock (e.g., aqueous organic feedstock) and microbial inoculum may be transferred to the first reactor separately. In some embodiments, the feedstock (e.g., aqueous organic feedstock) is transferred to the first reactor before the microbial inoculum. In some embodiments, the microbial inoculum is transferred to the first reactor before the feedstock (e.g., aqueous organic feedstock). In some embodiments, the feedstock (e.g., aqueous organic feedstock) and microbial inoculum may be transferred to the first reactor together.

A feedstock (e.g., organic feedstock) can comprise digestion products from digestion of organic substrates present in the feedstock (e.g., organic feedstock). Organic substrates may improve stability of the fluid feed mixture. Organic substrates can comprise coconut coir, peat moss, hemp, and/or wood fiber. In some embodiments, organic substrates comprise raw biomaterials present in the feedstock (e.g., aqueous organic feedstock). A bioreactor system may comprise a plurality of microbes and/or microorganisms derived from digestion of organic substrates in a feedstock (e.g., aqueous organic feedstock). An aqueous organic feedstock can be an organic feedstock comprising water. In some embodiments, the feedstock (e.g., aqueous organic feedstock) may further comprise a inorganic substrate. In some embodiments, the feedstock (e.g., aqueous organic feedstock) may comprise more than one inorganic substrate. The inorganic substrate may improve stability of the fluid feed mixture. In some embodiments, the inorganic substrate comprises sand, vermiculite, perlite, diatomaceous earth, and/or pumice. In some embodiments, the inorganic substrates comprises a mineral. In some embodiments, the inorganic substrate comprises rock phosphate. In some embodiments, the inorganic substrate can be zinc oxide.

A feedstock (e.g., organic feedstock) described herein may comprise various organic and/or biological materials. In some embodiments, the feedstock (e.g., organic feedstock) further comprises Saccharomyces cerevisiae yeast, Saccharomyces arboricola yeast, Saccharomyces mikatae yeast, Saccharomyces jurei yeast, Saccharomyces eubayanus yeast, Saccharomyces kudriavzevii yeast, Saccharomyces uvarum yeast, or any combination thereof. In some embodiments, the feedstock (e.g., organic feedstock) may be an aqueous mixture of at least one feedstock material and water. In some embodiments, the feedstock (e.g., organic feedstock) may be an aqueous mixture of cow manure, S. cerevisiae yeast, water, or any combination thereof.

In some embodiments, the feedstock (e.g., organic feedstock) is mixed within a reactor. In some embodiments, the feedstock (e.g., organic feedstock) is mixed outside of a reactor. In some embodiments, the feedstock (e.g., organic feedstock) is mixed between one, two, three, or more reactors. In some embodiments, the feedstock (e.g., organic feedstock) is a homogenous mixture.

In some embodiments, the feedstock (e.g., organic feedstock) further comprises a microbial consortium. The terms “microbe”, “microbial strain” and “microorganism” may refer to microscopic organisms, comprising bacteria, fungi, lichens, algae, protozoa, archaea, molds, or any combination thereof. The terms “microbe” and microorganism” may be used interchangeably herein. The feedstock (e.g., organic feedstock) can comprise a microbial consortium with 2, 3, 4, 5, 6, 7, 8, 9, 10, or more microorganisms. The feedstock (e.g., organic feedstock) can comprise a microbial consortium with 2, 3, 4, 5, 6, 7, 8, 9, 10, or more groups of microorganisms. The feedstock (e.g., organic feedstock) can comprise a microbial consortium with one group of microorganisms. The microbial consortium can comprise different microorganisms. The microbial consortium can comprise the same microorganism. The microbes in the consortia may be derived from the microbes originally present within the feedstock (e.g., organic feedstock). The microbes may digest the manure, yeast, other organic raw materials, or any combination thereof to produce digestion products.

In some embodiments, the aqueous feedstock may not contain the target isolate strain (e.g., an inoculum of the microbe). For example, the aqueous feedstock may not contain the target isolate strain prior to transfer to a first container. The concentration of the target isolate microbial strain may be 0 CFU/ml. In some embodiments, the aqueous feedstock may contain the target isolate strain prior to transfer to a first container. In some embodiments, the aqueous feedstock may contain at most about 5%, at most about 4%, at most about 3%, at most about 2%, at most about 1%, at most about 0.5%, at most about 0.1%, at most about 0.09%, at most about 0.08%, at most about 0.07%, at most about 0.06%, at most about 0.05%, at most about, 0.04%, at most about 0.03%, at most about 0.02%, at most about 0.01%, at most about 0.009%, at most about 0.008%, at most about 0.007%, at most about 0.006%, at most about 0.005%, at most about 0.004%, at most about 0.003%, at most about 0.002%, at most about 0.001%, at most about 0.0001%, or less than about 0.00001% of the target isolate strain. In some embodiments, the aqueous feedstock may contain at least about 0.00001%, at least about 0.0001%, at least about 0.001%, at least about 0.002%, at least about 0.003%, at least about 0.004%, at least about 0.005%, at least about 0.006%, at least about 0.007%, at least about 0.008%, at least about 0.009%, at least about 0.01%, at least about 0.02%, at least about 0.03%, at least about 0.04%, at least about 0.05%, at least about 0.06%, at least about 0.07%, at least about 0.08%, at least about 0.09%, at least about 0.1%, at least about 0.5%, at least about 1%, at least about 2%, at least about 3%, at least about 4%, or more than about 5% of the target isolate strain.

In some embodiments, the aqueous feedstock may not comprise the microbial strain (e.g., the zinc-solubilizing microbial strain, the phosphate-solubilizing microbial strain, the nitrogen use efficiency-promoting microbial strain) at a concentration higher than about 1 CFU/ml, higher than about 2 CFU/ml, higher than about 3 CFU/ml, higher than about 4 CFU/ml, higher than about 5 CFU/ml, higher than about 6 CFU/ml, higher than about 7 CFU/ml, higher than about 8 CFU/ml, higher than about 9 CFU/ml, higher than about 10 CFU/ml, higher than about 11 CFU/ml, higher than about 12 CFU/ml, higher than about 13 CFU/ml, higher than about 14 CFU/ml, higher than about 15 CFU/ml, higher than about 20 CFU/ml, higher than about 25 CFU/ml, higher than about 30 CFU/ml, higher than about 40 CFU/ml, or higher than about 50 CFU/ml. In some embodiments, the aqueous feedstock may not comprise the microbial strain (e.g., the zinc-solubilizing microbial strain, the phosphate-solubilizing microbial strain, the nitrogen use efficiency-promoting microbial strain) at a concentration less than about 50 CFU/ml, less than about 40 CFU/ml, less than about 30 CFU/ml, less than about 25 CFU/ml, less than about 20 CFU/ml, less than about 15 CFU/ml, less than about 14 CFU/ml, less than about 13 CFU/ml, less than about 12 CFU/ml, less than about 11 CFU/ml, less than about 10 CFU/ml, less than about 9 CFU/ml, less than about 8 CFU/ml, less than about 7 CFU/ml, less than about 6 CFU/ml, less than about 5 CFU/ml, less than about 4 CFU/ml, less than about 3 CFU/ml, less than about 2 CFU/ml, less than about 1 CFU/ml.

An inoculum of a microbe (e.g., a target microbial strain) may incubate in a reactor (e.g., container) of a bioreactor system described herein. In some embodiments, the inoculum of a microbe may be incubated under conditions that selectively enrich and/or maintain a concentration of the microbial strain in the digestions system. These conditions to selectively enrich and/or maintain a population of the microbial strain may comprise an addition of a selective pressure source (e.g., a zinc source, a phosphate source, a nitrogen source). In some cases, the population of the microbial strain may survive in the bioreactor system in a vegetative or sporulated state (e.g., a dormant state in the system). In some cases, the selective pressure source (e.g., selective pressure) may establish an environment to enhance the survival of the population of the microbial strain. Without wishing to be bound by theory, the conditions of reactors of a digestions system (e.g., a selective pressure source, a hydraulic retention time of the system (e.g., flow rate), floc recirculation, pH level of the system, aerobic conditions, or any combination thereof) may shift the complex microbial consortia of the bioreactor system to enrich at least a portion of microbes within a microbial consortium with plant growth promotion properties (e.g., zinc solubilization, phosphate solubilization, nitrogen use efficiency). Incubation of the inoculum of the microbe and/or the portion of microbes within a microbial consortium with plant growth promotion properties may further generate metabolites with plant growth promotion properties (e.g., zinc solubilization, phosphate solubilization, nitrogen use efficiency).

In some cases, the change in ionic concentration caused by the addition of the selective pressure may promote the growth of microbes or at least a portion of microbes in the microbial consortium. These microbes may be zinc-solubilizing microbes, phosphate-solubilizing microbes, nitrogen use efficiency microbes, or any combination thereof. An amount of microbes or at least a portion of microbes in the microbial consortium may grow in a measure (e.g., a concentration or a bacterial count) by at least about 0.0001%, at least about 0.001%, at least about 0.01%, at least about 0.1%, at least about 1%, at least about 2%, at least about 3%, at least about 4%, at least about 5%, at least about 10%, at least about 15%, at least about 20%, or at least about 30% following addition of the selective pressure. An amount of microbes or at least a portion of microbes in the microbial consortium may grow in a measure (e.g., a concentration or a bacterial count) by at most about 30%, at most about 20%, at most about 15%, at most about 10%, at most about 5%, at most about 4%, at most about 3%, at most about 2%, at most about 1%, at most about 0.1%, at most about 0.01%, at most about 0.001%, or at most about 0.0001% following addition of the selective pressure. An amount of microbes or at least a portion of microbes in the microbial consortium may grow in a measure (e.g., a concentration or a bacterial count) by about 0.0001% to about 0.001%, about 0.0001% to about 0.01%, about 0.0001% to about 0.1%, about 0.0001% to about 1%, about 0.0001% to about 2%, about 0.0001% to about 3%, about 0.0001% to about 4%, about 0.0001% to about 5%, about 0.0001% to about 10%, about 0.0001% to about 20%, about 0.0001% to about 30%, about 0.001% to about 0.01%, about 0.001% to about 0.1%, about 0.001% to about 1%, about 0.001% to about 2%, about 0.001% to about 3%, about 0.001% to about 4%, about 0.001% to about 5%, about 0.001% to about 10%, about 0.001% to about 20%, about 0.001% to about 30%, about 0.01% to about 0.1%, about 0.01% to about 1%, about 0.01% to about 2%, about 0.01% to about 3%, about 0.01% to about 4%, about 0.01% to about 5%, about 0.01% to about 10%, about 0.01% to about 20%, about 0.01% to about 30%, about 0.1% to about 1%, about 0.1% to about 2%, about 0.1% to about 3%, about 0.1% to about 4%, about 0.1% to about 5%, about 0.1% to about 10%, about 0.1% to about 20%, about 0.1% to about 30%, 1% to about 2%, about 1% to about 3%, about 1% to about 4%, about 1% to about 5%, about 1% to about 10%, about 1% to about 20%, about 1% to about 30%, about 2% to about 3%, about 2% to about 4%, about 2% to about 5%, about 2% to about 10%, about 2% to about 20%, about 2% to about 30%, about 3% to about 4%, about 3% to about 5%, about 3% to about 10%, about 3% to about 20%, about 3% to about 30%, about 4% to about 5%, about 4% to about 10%, about 4% to about 20%, about 4% to about 30%, about 5% to about 10%, about 5% to about 20%, about 5% to about 30%, about 10% to about 20%, about 10% to about 30%, or about 20% to about 30% following addition of the selective pressure. In some cases, at least a portion of zinc-solubilizing microbes in the microbial consortium may enrich and/or grow in the system without addition of the inoculum of the microbial strain. In some cases, at least a portion of phosphate-solubilizing microbes in the microbial consortium may enrich and/or grow in the system without addition of the inoculum of the microbial strain. In some cases, at least a portion of nitrogen use efficiency microbes in the microbial consortium may enrich and/or grow in the system without addition of the inoculum of the microbial strain.

A selective pressure source may be added to any container of a bioreactor system on a first day of a digestion process. In some embodiments, a selective pressure source may be added to the bioreactor system daily. In some embodiments, a selective pressure source may be added to the bioreactor system every 2 days, every 3 days, every 4 days, every 5 days, every 6 days, and/or weekly. In some embodiments, one selective pressure source is added. Alternatively, in some embodiments at least about 2, 3, 4, 5, or more differing selective pressure sources are added.

In some cases, parameters comprising nutrients added to the system, a hydraulic retention time of the system (e.g., flow rate), floc recirculation, pH level of the system, aerobic conditions, or any combination thereof may promote the growth of microbes or at least a portion of microbes in the microbial consortium. These microbes may be nitrogen use efficiency-promoting microbes. These microbes may be zinc use efficiency-promoting microbes. These microbes may be phosphate use efficiency-promoting microbes. An amount of microbes or at least a portion of microbes in the microbial consortium may grow in a measure (e.g., a concentration or a bacterial count) by at least about 0.0001%, at least about 0.001%, at least about 0.01%, at least about 0.1%, at least about 1%, at least about 2%, at least about 3%, at least about 4%, at least about 5%, at least about 10%, at least about 15%, at least about 20%, or at least about 30% as they incubate in conditions of the reactors of the bioreactor system (e.g., nutrients added to the system, a hydraulic retention time of the system (e.g., flow rate), floc recirculation, pH level of the system, aerobic conditions, or any combination thereof). An amount of microbes or at least a portion of microbes in the microbial consortium may grow in a measure (e.g., a concentration or a bacterial count) by at most about 30%, at most about 20%, at most about 15%, at most about 10%, at most about 5%, at most about 4%, at most about 3%, at most about 2%, at most about 1%, at most about 0.1%, at most about 0.01%, at most about 0.001%, or at most about 0.0001% as they incubate in conditions of the reactors of the bioreactor system (e.g., nutrients added to the system, a hydraulic retention time of the system (e.g., flow rate), floc recirculation, pH level of the system, aerobic conditions, or any combination thereof). An amount of microbes or at least a portion of microbes in the microbial consortium may grow in a measure (e.g., a concentration or a bacterial count) by about 0.0001% to about 0.001%, about 0.0001% to about 0.01%, about 0.0001% to about 0.1%, about 0.0001% to about 1%, about 0.0001% to about 2%, about 0.0001% to about 3%, about 0.0001% to about 4%, about 0.0001% to about 5%, about 0.0001% to about 10%, about 0.0001% to about 20%, about 0.0001% to about 30%, about 0.001% to about 0.01%, about 0.001% to about 0.1%, about 0.001% to about 1%, about 0.001% to about 2%, about 0.001% to about 3%, about 0.001% to about 4%, about 0.001% to about 5%, about 0.001% to about 10%, about 0.001% to about 20%, about 0.001% to about 30%, about 0.01% to about 0.1%, about 0.01% to about 1%, about 0.01% to about 2%, about 0.01% to about 3%, about 0.01% to about 4%, about 0.01% to about 5%, about 0.01% to about 10%, about 0.01% to about 20%, about 0.01% to about 30%, about 0.1% to about 1%, about 0.1% to about 2%, about 0.1% to about 3%, about 0.1% to about 4%, about 0.1% to about 5%, about 0.1% to about 10%, about 0.1% to about 20%, about 0.1% to about 30%, 1% to about 2%, about 1% to about 3%, about 1% to about 4%, about 1% to about 5%, about 1% to about 10%, about 1% to about 20%, about 1% to about 30%, about 2% to about 3%, about 2% to about 4%, about 2% to about 5%, about 2% to about 10%, about 2% to about 20%, about 2% to about 30%, about 3% to about 4%, about 3% to about 5%, about 3% to about 10%, about 3% to about 20%, about 3% to about 30%, about 4% to about 5%, about 4% to about 10%, about 4% to about 20%, about 4% to about 30%, about 5% to about 10%, about 5% to about 20%, about 5% to about 30%, about 10% to about 20%, about 10% to about 30%, or about 20% to about 30% as they incubate in conditions of the reactors of the bioreactor system (e.g., nutrients added to the system, a hydraulic retention time of the system (e.g., flow rate), floc recirculation, pH level of the system, aerobic conditions, or any combination thereof). In some cases, at least a portion of nitrogen use efficiency-promoting microbes in the microbial consortium may enrich and/or grow in the system without addition of the inoculum of the microbe. In some cases, at least a portion of zinc use efficiency-promoting microbes in the microbial consortium may enrich and/or grow in the system without addition of the inoculum of the microbe. In some cases, at least a portion of phosphate use efficiency-promoting microbes in the microbial consortium may enrich and/or grow in the system without addition of the inoculum of the microbe.

Without wishing to be bound by theory, parameters comprising nutrients added to the system, a hydraulic retention time of the system (e.g., flow rate), floc recirculation, pH level of the system, aerobic conditions, or any combination thereof, may inhibit growth of a microbe and/or a population of microbes. Without wishing to be bound by theory, parameters comprising nutrients added to the system, a hydraulic retention time of the system (e.g., flow rate), floc recirculation, pH level of the system, aerobic conditions, or any combination thereof, may inhibit growth of a microbe and/or a population of microbes and enhance growth of a target microbe and/or target population of microbes (e.g., nitrogen use efficiency-promoting microbes, phosphate solubilizing microbes, zinc solubilizing microbes). Without wishing to be bound by theory, the parameters of the bioreactor system may cause a selective shift in a microbial population to favor microbes with a targeted functionality (e.g., nitrogen use efficiency, zinc solubilization, phosphate solubilization). Nutrients (e.g., macronutrients, micronutrients, inorganic nutrients, or any combination thereof) can be present in the bioreactor system to provide an environment for bacterial growth.

An inoculum of a microbe described herein may contact (e.g., be applied to) a plant. In some embodiments, the contacting of an inoculum of a microbe to a plant may enhance at least one plant growth promotion property of the plant. In some embodiments, one, two, three, four, or more inoculums of a microbe may be transferred to a bioreactor system. An inoculum of a microbe and another inoculum of a microbe may be the same. An inoculum of a microbe and another inoculum of a microbe may be different. In some embodiments, the inoculum of the microbe and the feedstock (e.g., aqueous organic feedstock) are transferred to a container of the bioreactor system at the same time. In some embodiments, the inoculum of the microbe and the feedstock (e.g., aqueous organic feedstock) are not transferred to a container of the bioreactor system at the same time. In some embodiments, the inoculum of the microbe is transferred to a container of the bioreactor system prior to the feedstock (e.g., aqueous organic feedstock). In some embodiments, the inoculum of the microbe is transferred to a container of the bioreactor system after the feedstock (e.g., aqueous organic feedstock).

In some embodiments, as a working fluid flows through a bioreactor system, an absolute number of microbes may increase. In some embodiments, the absolute number of microbes may stay substantially the same (e.g., within 80% of a reference number) or decrease, but activity of microbes in the system may increase. The amount of microbes in a fluid may be measured by plating a portion of the fluid on culture plates with a media containing the relevant selective pressure (e.g., an insoluble nutrient as the only source of that nutrient) and counting the number of colonies that form halos. An absolute number of microbes can remain substantially the same if, between measures at two time points, an amount of microbes (e.g., a number of microbes) may not change by at most about 30%, 25%, 20%, 18%, 15%, 12%, 10%, 8%, 5%, 4%, 3%, 2%, 1%, or less than about 1%.

An inoculum of a microbe may generate metabolites in a bioreactor system as described herein. Microbes of the microbial consortium and/or the inoculum of the microbe may be metabolized by catalytic enzymes to produce metabolites. Metabolites may be generated by microbial metabolism. Metabolites may be generated by enzymes catalyzing biochemical reactions of the organic substrates of the feedstock (e.g., aqueous organic feedstock) in a working fluid of a bioreactor system as described herein. The metabolites generated by the inoculum of the microbe may have a plant growth promotion property. The metabolites generated by the inoculum of the microbe may have two or more plant growth promotion properties. The plant growth promotion properties may comprise shoot biomass, root biomass, nutrient uptake, crop yield, deaminase activity, acid production, leaf area, chlorophyll content, heat tolerance, cold tolerance, drought tolerance, salt tolerance, total biomass, or any combination thereof. Metabolites may be used in biostimulant compositions and/or may be applied to plants.

In some embodiments, the feedstock (e.g., aqueous organic feedstock) comprises metabolites. In some embodiments, the feedstock (e.g., aqueous organic feedstock) comprises metabolites produced by microbes endogenous to the feedstock (e.g., organic feedstock). Primary metabolites can comprise carbohydrates, sugars, steroids, proteins, fats, vitamins, pigments, odorants, antibiotics, antioxidants, pheromones, nucleic acid components, and other small molecules. Metabolites can further comprise alkaloids, amino acids, biogenic amines, carboxylic acids, cresols, terpenoids, phenols (e.g., flavonoids, coumarins, tannins, lignans, stilbenes, or chromones), polyketides, eicosanoids, hormones or derivatives thereof, indoles or derivatives thereof, nucleobases, citric acid, ceramides, diglycerides, triglycerides, amides, alkanes, alcohols, stearates, sterols, polyols, organic acids, fatty acids, or any combination thereof. In some embodiments, metabolites comprise sugars and/or fatty acids. Sugars can comprise fructose, hexose, galactose, glucose, lactose, maltose, sucrose, xylose, or any combination thereof. Fatty acids can comprise stearic acid, lauric acid, myristic acid, palmitic acid, octadecenoic acid, octadecadienoic acid, oleic acid, arachidic acid, behenic acid, erucic acid, adrenic acid, tricosanoic acid, lignoceric acid, nervonic acid, nonadecanoic acid, arachidic acid, myristolic acid, hydroxylated myristic acid, or any combination thereof.

Metabolites generated by the inoculum of the microbe or by at least a portion of microbes of the microbial consortium may be present in a supernatant of a bioreactor system. In some embodiments, the metabolites may be present by weight in a volume of solution (e.g., in mg in 100 ml). In some embodiments, a weight of metabolites per 100 ml of a base product solution can be at least about 10 mg, at least about 20 mg, at least about 30 mg, at least about 40 mg, at least about 50 mg, at least about 60 mg, at least about 70 mg, at least about 80 mg, at least about 90 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, at least about 400 mg, at least about 500 mg, or greater than about 500 mg. In some embodiments, a weight of metabolites per 100 ml of a base product solution can be at most about 500 mg, at most about 400 mg, at most about 300 mg, at most about 280 mg, at most about 260 mg, at most about 240 mg, at most about 220 mg, at most about 200 mg, at most about 180 mg, at most about 160 mg, at most about 140 mg, at most about 120 mg, at most about 100 mg, at most about 90 mg, at most about 80 mg, at most about 70 mg, at most about 60 mg, at most about 50 mg, at most about 40 mg, at most about 30 mg, at most about 20 mg, at most about 10 mg, or less than about 10 mg. In some embodiments, a weight of metabolites per 100 ml of a base product solution can be from about 10 mg to about 500 mg. In some embodiments, a weight of metabolites per 100 ml of a base product solution can be from about 10 mg to about 20 mg, about 10 mg to about 30 mg, about 10 mg to about 40 mg, about 10 mg to about 50 mg, about 10 mg to about 75 mg, about 10 mg to about 100 mg, about 10 mg to about 125 mg, about 10 mg to about 150 mg, about 10 mg to about 175 mg, about 10 mg to about 250 mg, about 10 mg to about 500 mg, about 20 mg to about 30 mg, about 20 mg to about 40 mg, about 20 mg to about 50 mg, about 20 mg to about 75 mg, about 20 mg to about 100 mg, about 20 mg to about 125 mg, about 20 mg to about 150 mg, about 20 mg to about 175 mg, about 20 mg to about 250 mg, about 20 mg to about 500 mg, about 30 mg to about 40 mg, about 30 mg to about 50 mg, about 30 mg to about 75 mg, about 30 mg to about 100 mg, about 30 mg to about 125 mg, about 30 mg to about 150 mg, about 30 mg to about 175 mg, about 30 mg to about 250 mg, about 30 mg to about 500 mg, about 40 mg to about 50 mg, about 40 mg to about 75 mg, about 40 mg to about 100 mg, about 40 mg to about 125 mg, about 40 mg to about 150 mg, about 40 mg to about 175 mg, about 40 mg to about 250 mg, about 40 mg to about 500 mg, about 50 mg to about 75 mg, about 50 mg to about 100 mg, about 50 mg to about 125 mg, about 50 mg to about 150 mg, about 50 mg to about 175 mg, about 50 mg to about 250 mg, about 50 mg to about 500 mg, about 75 mg to about 100 mg, about 75 mg to about 125 mg, about 75 mg to about 150 mg, about 75 mg to about 175 mg, about 75 mg to about 250 mg, about 75 mg to about 500 mg, about 100 mg to about 125 mg, about 100 mg to about 150 mg, about 100 mg to about 175 mg, about 100 mg to about 250 mg, about 100 mg to about 500 mg, about 125 mg to about 150 mg, about 125 mg to about 175 mg, about 125 mg to about 250 mg, about 125 mg to about 500 mg, about 150 mg to about 175 mg, about 150 mg to about 250 mg, about 150 mg to about 500 mg, about 175 mg to about 250 mg, about 175 mg to about 500 mg, or about 250 mg to about 500 mg.

The term “feedstock” (e.g., “organic feedstock”) described herein can refer to raw biomaterials such as carbon compounds, proteins, and/or carbohydrates. The feedstock may be organic feedstock. The feedstock may be inorganic feedstock. The organic feedstock may be an aqueous organic feedstock. In some cases, the organic feedstock may comprise one or more inorganic nutrients. In some embodiments, the feedstock (e.g., organic feedstock) may comprise organic substrates comprising cottonseed, algae, neem, orange seed, linseed, jojoba, kusum, rubber seed, alfalfa, sugarcane, Opuntia, coffee, Deccan hemp, or any combination thereof. In some embodiments, the feedstock can comprise an inorganic feedstock. In some embodiments, the feedstock (e.g., organic feedstock) may include, but is not limited to, manure, kelp, lignocellulose, wastewater biosolids, food waste, energy crops, glucose solution, ammonium sulfate, oils, fats, grease, or any combination thereof. In some embodiments, the feedstock is added at the beginning of the system (e.g., into a first reactor and/or a CMR). In some embodiments, the feedstock is added in a middle reactor of the system (e.g., not in the first or last reactor of the system). In some embodiments, the feedstock is added once to the system. In some embodiments, the feedstock is added two, three, four, or more times to the system. In some embodiments, the feedstock is added continuously to the system. In some embodiments, the feedstock (e.g., organic feedstock) is a composition of one raw biomaterial. In some embodiments, the feedstock (e.g., organic feedstock) is a blend of two, three, four, five, six, seven, eight, nine, ten, or more biomaterials.

Feedstock (e.g., organic feedstock) comprising carbon and nitrogen sources can flow into a reactor tank. Feedstock (e.g., organic feedstock) comprising carbon and nitrogen sources can flow into a reactor tank via a conduit (e.g., a pipe). The feedstock (e.g., organic feedstock) may further comprise microbial strain having a desired plant growth promoting property (e.g., a zinc-solubilizing microbial strain, a phosphate-solubilizing microbial strain, a nitrogen use efficiency microbial strain). In some embodiments, the feedstock (e.g., aqueous feedstock or aqueous organic feedstock) can comprise a concentration of the microbial strain (e.g., the zinc-solubilizing microbial strain, the phosphate-solubilizing microbial strain, the nitrogen use efficiency strain) of at most about 15 CFU/ml, at most about 14 CFU/ml, at most about 13 CFU/ml, at most about 12 CFU/ml, at most about 11 CFU/ml, at most about 10 CFU/ml, at most about 9 CFU/ml, at most about 8 CFU/ml, at most about 7 CFU/ml, at most about 6 CFU/ml, at most about 5 CFU/ml, at most about 4 CFU/ml, at most about 3 CFU/ml, at most about 2 CFU/ml, at most about 1 CFU/ml, or less than about 1 CFU/ml. In some embodiments, the feedstock (e.g., aqueous feedstock or aqueous organic feedstock) can comprise a concentration of the microbial strain (e.g., the zinc-solubilizing microbial strain, the phosphate-solubilizing microbial strain, the nitrogen use efficiency strain) of at least about 1 CFU/ml, at least about 2 CFU/ml, at least about 3 CFU/ml, at least about 4 CFU/ml, at least about 5 CFU/ml, at least about 6 CFU/ml, at least about 7 CFU/ml, at least about 8 CFU/ml, at least about 9 CFU/ml, at least about 10 CFU/ml, at least about 11 CFU/ml, at least about 12 CFU/ml, at least about 13 CFU/ml, at least about 14 CFU/ml, at least about 15 CFU/ml. In some embodiments, the feedstock (e.g., aqueous feedstock or aqueous organic feedstock) can comprise a concentration of the microbial strain (e.g., the zinc-solubilizing microbial strain, the phosphate-solubilizing microbial strain, the nitrogen use efficiency strain) of about 1 CFU/ml to about 2 CFU/ml, about 1 CFU/ml to about 3 CFU/ml, about 1 CFU/ml to about 4 CFU/ml, about 1 CFU/ml to about 5 CFU/ml, about 1 CFU/ml to about 6 CFU/ml, about 1 CFU/ml to about 7 CFU/ml, about 1 CFU/ml to about 8 CFU/ml, about 1 CFU/ml to about 9 CFU/ml, about 1 CFU/ml to about 10 CFU/ml, about 1 CFU/ml to about 11 CFU/ml, about 1 CFU/ml to about 12 CFU/ml, about 1 CFU/ml to about 13 CFU/ml, about 1 CFU/ml to about 14 CFU/ml, about 1 CFU/ml to about 15 CFU/ml, about 2 CFU/ml to about 3 CFU/ml, about 2 CFU/ml to about 4 CFU/ml, about 2 CFU/ml to about 5 CFU/ml, about 2 CFU/ml to about 6 CFU/ml, about 2 CFU/ml to about 7 CFU/ml, about 2 CFU/ml to about 8 CFU/ml, about 2 CFU/ml to about 9 CFU/ml, about 2 CFU/ml to about 10 CFU/ml, about 2 CFU/ml to about 11 CFU/ml, about 2 CFU/ml to about 12 CFU/ml, about 2 CFU/ml to about 13 CFU/ml, about 2 CFU/ml to about 14 CFU/ml, about 2 CFU/ml to about 15 CFU/ml, about 3 CFU/ml to about 4 CFU/ml, about 3 CFU/ml to about 5 CFU/ml, about 3 CFU/ml to about 6 CFU/ml, about 3 CFU/ml to about 7 CFU/ml, about 3 CFU/ml to about 8 CFU/ml, about 3 CFU/ml to about 9 CFU/ml, about 3 CFU/ml to about 10 CFU/ml, about 3 CFU/ml to about 11 CFU/ml, about 3 CFU/ml to about 12 CFU/ml, about 3 CFU/ml to about 13 CFU/ml, about 3 CFU/ml to about 14 CFU/ml, about 3 CFU/ml to about 15 CFU/ml, about 4 CFU/ml to about 5 CFU/ml, about 4 CFU/ml to about 6 CFU/ml, about 4 CFU/ml to about 7 CFU/ml, about 4 CFU/ml to about 8 CFU/ml, about 4 CFU/ml to about 9 CFU/ml, about 4 CFU/ml to about 10 CFU/ml, about 4 CFU/ml to about 11 CFU/ml, about 4 CFU/ml to about 12 CFU/ml, about 4 CFU/ml to about 13 CFU/ml, about 4 CFU/ml to about 14 CFU/ml, about 4 CFU/ml to about 15 CFU/ml, about 5 CFU/ml to about 6 CFU/ml, about 5 CFU/ml to about 7 CFU/ml, about 5 CFU/ml to about 8 CFU/ml, about 5 CFU/ml to about 9 CFU/ml, about 5 CFU/ml to about 10 CFU/ml, about 5 CFU/ml to about 11 CFU/ml, about 5 CFU/ml to about 12 CFU/ml, about 5 CFU/ml to about 13 CFU/ml, about 5 CFU/ml to about 14 CFU/ml, about 5 CFU/ml to about 15 CFU/ml, about 6 CFU/ml to about 7 CFU/ml, about 6 CFU/ml to about 8 CFU/ml, about 6 CFU/ml to about 9 CFU/ml, about 6 CFU/ml to about 10 CFU/ml, about 6 CFU/ml to about 11 CFU/ml, about 6 CFU/ml to about 12 CFU/ml, about 6 CFU/ml to about 13 CFU/ml, about 6 CFU/ml to about 14 CFU/ml, about 6 CFU/ml to about 15 CFU/ml, about 7 CFU/ml to about 8 CFU/ml, about 7 CFU/ml to about 9 CFU/ml, about 7 CFU/ml to about 10 CFU/ml, about 7 CFU/ml to about 11 CFU/ml, about 7 CFU/ml to about 12 CFU/ml, about 7 CFU/ml to about 13 CFU/ml, about 7 CFU/ml to about 14 CFU/ml, about 7 CFU/ml to about 15 CFU/ml, about 8 CFU/ml to about 9 CFU/ml, about 8 CFU/ml to about 10 CFU/ml, about 8 CFU/ml to about 11 CFU/ml, about 8 CFU/ml to about 12 CFU/ml, about 8 CFU/ml to about 13 CFU/ml, about 8 CFU/ml to about 14 CFU/ml, about 8 CFU/ml to about 15 CFU/ml, about 9 CFU/ml to about 10 CFU/ml, about 9 CFU/ml to about 11 CFU/ml, about 9 CFU/ml to about 12 CFU/ml, about 9 CFU/ml to about 13 CFU/ml, about 9 CFU/ml to about 14 CFU/ml, about 9 CFU/ml to about 15 CFU/ml, about 10 CFU/ml to about 11 CFU/ml, about 10 CFU/ml to about 12 CFU/ml, about 10 CFU/ml to about 13 CFU/ml, about 10 CFU/ml to about 14 CFU/ml, about 10 CFU/ml to about 15 CFU/ml, about 11 CFU/ml to about 12 CFU/ml, about 11 CFU/ml to about 13 CFU/ml, about 11 CFU/ml to about 14 CFU/ml, about 11 CFU/ml to about 15 CFU/ml, about 12 CFU/ml to about 13 CFU/ml, about 12 CFU/ml to about 14 CFU/ml, about 12 CFU/ml to about 15 CFU/ml, about 13 CFU/ml to about 14 CFU/ml, about 13 CFU/ml to about 15 CFU/ml, or about 14 CFU/ml to about 15 CFU/ml.

The inoculum of a microbe as described herein may metabolize the nitrogen source. Metabolism of nitrogen by the inoculum of the microbe may comprise transfer of nitrogen-based moieties of the nitrogen source to substrates in the working fluid. In some embodiments, the carbon source may be transferred to a first container of the bioreactor system. In some embodiments, the carbon source may be transferred to a second, third, fourth, fifth, or sixth container of the bioreactor system. In some embodiments, the nitrogen source may be transferred to a first container of the bioreactor system. In some embodiments, the nitrogen source may be transferred to a second, third, fourth, fifth, or sixth container of the bioreactor system.

In some embodiments, microbes can be added to the start of the system (e.g., into the first reactor). In some embodiments, microbes can be added to the middle of the system (e.g., into a reactor that is not the first reactor or the final reactor of the system) and/or microbes can be added to the end of the system (e.g., into the final reactor). Microbes can be added concurrently with the feedstock (e.g., organic feedstock). Microbes can be added separately from the feedstock (e.g., organic feedstock). In some embodiments, microbes may be added to the system with the feedstock (e.g., organic feedstock) in the same reactor. In some embodiments, microbes may be added to the system with the feedstock (e.g., organic feedstock) in different reactors. In some embodiments, microbes may be added to the system prior to the feedstock (e.g., organic feedstock). In some embodiments, microbes may be added to the system after the feedstock (e.g., organic feedstock). In some embodiments, a period of time between addition of microbes to the system and addition of feedstock (e.g., organic feedstock) to the system can be at least about 30 seconds, at least about 1 minute, at least about 5 minutes, at least about 10 minutes, at least about 15 minutes, at least about 20 minutes, at least about 30 minutes, at least about 40 minutes, at least about 45 minutes, at least about 50 minutes, at least about 1 hour, at least about 2 hours, at least about 3 hours, at least about 4 hours, at least about 5 hours, at least about 6 hours, at least about 7 hours, at least about 8 hours, at least about 9 hours, or at least about 10 hours. In some embodiments, a period of time between addition of microbes to the system and addition of feedstock (e.g., organic feedstock) to the system can be at most about 10 hours, at most about 9 hours, at most about 8 hours, at most about 7 hours, at most about 6 hours, at most about 5 hours, at most about 4 hours, at most about 3 hours, at most about 2 hours, at most about 1 hours, at most about 50 minutes, at most about 45 minutes, at most about 40 minutes, at most about 30 minutes, at most about 20 minutes, at most about 15 minutes, at most about 10 minutes, at most about 5 minutes, at most about 1 minute, or at most about 30 seconds.

A microbe may have nutrient solubilization properties and/or plant growth promotion properties. For example, a microbe may increase plant growth, increase shoot and/or root biomass, increase crop yield, increase soil enzymatic activity, increase photosynthesis efficiency, lower heavy metal uptake, and/or decrease soil pH. A microbe may enhance plant growth on land with high salinity, on land with heavy metal contamination, or on land with drought conditions. A microbe (e.g., isolate) described herein may have zinc solubilization properties.

In some embodiments, the bioreactor system may comprise a retention time. A retention time may comprise a time an inoculum of a microbe spends in a bioreactor system or a time an inoculum of a microbe spends following transfer into a first container and until collection from the bioreactor system. A longer retention time may be advantageous for growth or enrichment of an inoculum of a microbe of the bioreactor system. A shorter retention time may be advantageous for growth or enrichment of an inoculum of a microbe of the bioreactor system. A retention time of a bioreactor system may comprise at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 1 month, at least about 2 months, at least about 3 months, at least about 6 months, at least about 9 months, at least about 1 year, at least about 2 years, at least about 3 years, at least about 4 years, at least about 5 years, or at least about 10 years. A retention time of a bioreactor system may comprise at most about 10 years, at most about 5 years, at most about 4 years, at most about 3 years, at most about 2 years, at most about 1 year, at most about 9 months, at most about 6 months, at most about 3 months, at most about 2 months, at most about 1 month, at most about 4 weeks, at most about 3 weeks, at most about 2 weeks, at most about 1 weeks, at most about 6 days, at most about 5 days, at most about 4 days, at most about 3 days, at most about 2 days, or at most about 1 day. A retention time of a bioreactor system may comprise about 1 day to about 2 days, about 1 day to about 3 days, about 1 day to about 4 days, about 1 day to about 5 days, about 1 day to about 6 days, about 1 day to about 1 week, about 1 day to about 2 weeks, about 1 day to about 3 weeks, about 1 day to about 4 weeks, about 1 day to about 1 month, about 1 day to about 2 months, about 1 day to about 3 months, about 1 day to about 6 months, about 1 day to about 9 months, about 1 day to about 1 year, about 1 day to about 2 years, about 1 day to about 3 years, about 1 day to about 4 years, about 1 day to about 5 years, about 1 day to about 10 years, about 2 days to about 3 days, about 2 days to about 4 days, about 2 days to about 5 days, about 2 days to about 6 days, about 2 days to about 1 week, about 2 days to about 2 weeks, about 2 days to about 3 weeks, about 2 days to about 4 weeks, about 2 days to about 1 month, about 2 days to about 2 months, about 2 days to about 3 months, about 2 days to about 6 months, about 2 days to about 9 months, about 2 days to about 1 year, about 2 days to about 2 years, about 2 days to about 3 years, about 2 days to about 4 years, about 2 days to about 5 years, about 2 days to about 10 years, about 3 days to about 4 days, about 3 days to about 5 days, about 3 days to about 6 days, about 3 days to about 1 week, about 3 days to about 2 weeks, about 3 days to about 3 weeks, about 3 days to about 4 weeks, about 3 days to about 1 month, about 3 days to about 2 months, about 3 days to about 3 months, about 3 days to about 6 months, about 3 days to about 9 months, about 3 days to about 1 year, about 3 days to about 2 years, about 3 days to about 3 years, about 3 days to about 4 years, about 3 days to about 5 years, about 3 days to about 10 years, about 4 days to about 5 days, about 4 days to about 6 days, about 4 days to about 1 week, about 4 days to about 2 weeks, about 4 days to about 3 weeks, about 4 days to about 4 weeks, about 4 days to about 1 month, about 4 days to about 2 months, about 4 days to about 3 months, about 4 days to about 6 months, about 4 days to about 9 months, about 4 days to about 1 year, about 4 days to about 2 years, about 4 days to about 3 years, about 4 days to about 4 years, about 4 days to about 5 years, about 4 days to about 10 years, about 5 days to about 6 days, about 5 days to about 1 week, about 5 days to about 2 weeks, about 5 days to about 3 weeks, about 5 days to about 4 weeks, about 5 days to about 1 month, about 5 days to about 2 months, about 5 days to about 3 months, about 5 days to about 6 months, about 5 days to about 9 months, about 5 days to about 1 year, about 5 days to about 2 years, about 5 days to about 3 years, about 5 days to about 4 years, about 5 days to about 5 years, about 5 days to about 10 years, about 6 days to about 1 week, about 6 days to about 2 weeks, about 6 days to about 3 weeks, about 6 days to about 4 weeks, about 6 days to about 1 month, about 6 days to about 2 months, about 6 days to about 3 months, about 6 days to about 6 months, about 6 days to about 9 months, about 6 days to about 1 year, about 6 days to about 2 years, about 6 days to about 3 years, about 6 days to about 4 years, about 6 days to about 5 years, about 6 days to about 10 years, about 1 week to about 2 weeks, about 1 week to about 3 weeks, about 1 week to about 4 weeks, about 1 week to about 1 month, about 1 week to about 2 months, about 1 week to about 3 months, about 1 week to about 6 months, about 1 week to about 9 months, about 1 week to about 1 year, about 1 week to about 2 years, about 1 week to about 3 years, about 1 week to about 4 years, about 1 week to about 5 years, about 1 week to about 10 years, about 2 weeks to about 3 weeks, about 2 weeks to about 4 weeks, about 2 weeks to about 1 month, about 2 weeks to about 2 months, about 2 weeks to about 3 months, about 2 weeks to about 6 months, about 2 weeks to about 9 months, about 2 weeks to about 1 year, about 2 weeks to about 2 years, about 2 weeks to about 3 years, about 2 weeks to about 4 years, about 2 weeks to about 5 years, about 2 weeks to about 10 years, about 3 weeks to about 4 weeks, about 3 weeks to about 1 month, about 3 weeks to about 2 months, about 3 weeks to about 3 months, about 3 weeks to about 6 months, about 3 weeks to about 9 months, about 3 weeks to about 1 year, about 3 weeks to about 2 years, about 3 weeks to about 3 years, about 3 weeks to about 4 years, about 3 weeks to about 5 years, about 3 weeks to about 10 years, about 4 weeks to about 1 month, about 4 weeks to about 2 months, about 4 weeks to about 3 months, about 4 weeks to about 6 months, about 4 weeks to about 9 months, about 4 weeks to about 1 year, about 4 weeks to about 2 years, about 4 weeks to about 3 years, about 4 weeks to about 4 years, about 4 weeks to about 5 years, about 4 weeks to about 10 years, about 1 month to about 2 months, about 1 month to about 3 months, about 1 month to about 6 months, about 1 month to about 9 months, about 1 month to about 1 year, about 1 month to about 2 years, about 1 month to about 3 years, about 1 month to about 4 years, about 1 month to about 5 years, about 1 month to about 10 years, about 2 months to about 3 months, about 2 months to about 6 months, about 2 months to about 9 months, about 2 months to about 1 year, about 2 months to about 2 years, about 2 months to about 3 years, about 2 months to about 4 years, about 2 months to about 5 years, about 2 months to about 10 years, about 3 months to about 6 months, about 3 months to about 9 months, about 3 months to about 1 year, about 3 months to about 2 years, about 3 months to about 3 years, about 3 months to about 4 years, about 3 months to about 5 years, about 3 months to about 10 years, about 6 months to about 9 months, about 6 months to about 1 year, about 6 months to about 2 years, about 6 months to about 3 years, about 6 months to about 4 years, about 6 months to about 5 years, about 6 months to about 10 years, about 9 months to about 1 year, about 9 months to about 2 years, about 9 months to about 3 years, about 9 months to about 4 years, about 9 months to about 5 years, about 9 months to about 10 years, about 1 year to about 2 years, about 1 year to about 3 years, about 1 year to about 4 years, about 1 year to about 5 years, about 1 year to about 10 years, about 2 years to about 3 years, about 2 years to about 4 years, about 2 years to about 5 years, about 2 years to about 10 years, about 3 years to about 4 years, about 3 years to about 5 years, about 3 years to about 10 years, about 4 years to about 5 years, about 4 years to about 10 years, or about 5 years to about 10 years.

### B. Zinc System

Different steps in digestion of a feedstock may be performed in different reactors as described herein, which may result in (1) a more complete digestion—i.e., a more complete breakdown of macromolecules in the feedstock—than other types of bioreactor systems, and/or (2) production of a variety of microbial digestion products having plant growth promoting properties (e.g., ability to enhance or improve soil available zinc via zinc solubilization, zinc uptake by plant tissues, and/or otherwise promote zinc use efficiency).

In some embodiments, the bioreactor system (e.g., the bioreactor system) comprises an established population of one or more zinc-solubilizing microbial strains in one or more containers of the system. In some embodiments, a bioreactor system (e.g., one or more containers of a bioreactor system) comprises at least one zinc-solubilizing microbial strain. In some embodiments, a bioreactor system (e.g., one or more containers of a bioreactor system) comprises an established population of a first zinc-solubilizing microbial strain and an established population of a second zinc-solubilizing microbial strain. In some embodiments, the bioreactor system further comprises an established population of a third zinc-solubilizing microbial strain.

An inoculum of a microbe (e.g., a target microbial strain) may incubate in a reactor (e.g., container) of a bioreactor system described herein. In some embodiments, the inoculum of a microbe may be incubated under conditions that selectively enrich and/or maintain a concentration of the microbial strain in the digestions system. These conditions to selectively enrich and/or maintain a population of the microbial strain may comprise an addition of a selective pressure source (e.g., a zinc source). In some cases, the selective pressure can be a zinc source. In some case, the zinc source may be a form of insoluble zinc. Upon addition of a selective pressure (e.g., zinc source), the inoculation of a zinc-solubilizing microbial strain may solubilize the insoluble zinc and create a concentration of high zinc ion. This environment of high zinc ion concentration may further enrich a microbial population with a target functionality (e.g., zinc solubilization).

The inoculum of the microbe may comprise a zinc-solubilizing microbe that can be maintained at a higher ion concentration in a working fluid caused by addition of the selective pressure. Survival of an inoculum of a microbe may comprise the inoculum of a microbe configured to maintain its initial amount in the environment caused by addition of the selective pressure source. Survival of an inoculum of a microbe may comprise an instance where an amount of the inoculum of the microbe is alive (e.g., maintained) at the end of a retention period of the bioreactor system (e.g., in a reactor or clarifier container). Without wishing to be bound by theory, the inoculum of a microbe may not be susceptible to the change in ionic concentration or may have solubilizing properties for the ion which may allow it to survive in the working fluid. Following addition of the selective pressure, at least a portion of microbes of the microbial consortium may enrich (e.g., grow or increase in number). These microbes of the portion of microbes in the microbial consortium may have zinc solubilization capacities. Following addition of the selective pressure source, other microbes of the working fluid may reduce in number as the microbes of the microbial consortium are unable to survive (e.g., are susceptible) in the ionic environment. A proportion of the zinc-solubilizing microbial strain relative to at least a portion of the microbes in a microbial consortium may be maintained in a first container of a bioreactor system. A proportion of the zinc-solubilizing microbial strain relative to at least a portion of the microbes in a microbial consortium may be maintained in a second, third, fourth, fifth, sixth, seventh, or eighth container of a bioreactor system. A maintained zinc-solubilizing microbial strain may change its amount in a working fluid of a bioreactor system less than about 0.001%, less than about 0.01%, less than about 0.1%, less than about 0.5%, less than about 1%, less than about 5%, or less than about 10% over a measured time period.

Without wishing to be bound by theory, a high zinc concentration may inhibit growth of a microbe and/or a population of microbes. A high zinc concentration may inhibit growth of a microbe and/or a population of microbes unless the zinc can be solubilized. In some embodiments, the growth of a microbe and/or one or more populations of microbes may be inhibited in the presence of solubilized zinc if the microbe and/or one or more populations of microbes are not tolerant to the soluble high zinc concentration. A high zinc concentration can comprise at least about 50, 100, 200, 300, 400, 500, 600, 700, 800, 900, 1000, 1200, 1500, or greater than about 1500 mg/L (ppm) of zinc. In the presence of the high zinc concentration, zinc-tolerant microbes (e.g., zinc-solubilizing microbes) may be enriched in the microbial community. For example, zinc-solubilizing microbes may solubilize insoluble zinc and create an environment in which (i) the zinc-solubilizing microbes may become enriched, (ii) microbes that are intolerant to the high zinc (e.g., the solubilized and/or free zinc) may show reduced growth, or (iii) any combination thereof. Nutrients (e.g., macronutrients, micronutrients, inorganic nutrients, or any combination thereof) can be present in the bioreactor system to provide an environment for bacterial growth. The addition of a selective pressure (e.g., increased zinc ionic concentration) with inputs to facilitate microbial growth can allow for enrichment of zinc solubilizers in the working fluid of a bioreactor system described herein. The zinc solubilizers can comprise the inoculum of the microbe (e.g., a population of the inoculum of the microbe), zinc-solubilizing microbes of a microbial consortium, zinc-solubilizing metabolites produced by the inoculum of the microbe and/or the zinc-solubilizing microbes of the microbial consortium, or any combination thereof.

In some embodiments, the selective pressure can enrich a population of zinc-solubilizing microbes (e.g., zinc-solubilizer) in a container and/or containers of a bioreactor system. A population of zinc-solubilizing microbes can comprise an inoculum of a target zinc-solubilizing microbe, zinc-solubilizing microbes of a microbial consortium, zinc-solubilizing metabolites, or any combination thereof.

As a working fluid flows through a bioreactor system, an absolute number of zinc-solubilizing microbes may increase. In some embodiments, an absolute number of zinc solubilizers can be higher in a second container compared an absolute number of zinc solubilizers in a first container. In some embodiments, an absolute number of zinc solubilizers can be higher in a third container compared an absolute number of zinc solubilizers in a first container. In some embodiments, an absolute number of zinc solubilizers can be higher in a fourth container compared an absolute number of zinc solubilizers in a first container. In some embodiments, an absolute number of zinc solubilizers can be higher in a fifth container compared an absolute number of zinc solubilizers in a first container. In some embodiments, an absolute number of zinc solubilizers can be higher in a sixth container compared an absolute number of zinc solubilizers in a first container. In some embodiments, an absolute number of zinc solubilizers can be higher in a seventh container compared an absolute number of zinc solubilizers in a first container. In some embodiments, an absolute number of zinc solubilizers can be higher in an eighth container compared an absolute number of zinc solubilizers in a first container. In some embodiments, an absolute number of zinc solubilizers can be higher in a ninth container compared an absolute number of zinc solubilizers in a first container. In some embodiments, an absolute number of zinc solubilizers can be higher in a tenth container compared an absolute number of zinc solubilizers in a first container.

As working fluid flows through a bioreactor system, a proportion of zinc-solubilizer relative to a total population of bacteria may increase. In some embodiments, a proportion of zinc-solubilizer relative to a total population of bacteria can be higher in a second container compared a proportion of zinc-solubilizer relative to a total population of bacteria in a first container. In some embodiments, a proportion of zinc-solubilizer relative to a total population of bacteria can be higher in a third container compared a proportion of zinc-solubilizer relative to a total population of bacteria in a first container. In some embodiments a proportion of zinc-solubilizer relative to a total population of bacteria can be higher in a fourth container compared a proportion of zinc-solubilizer relative to a total population of bacteria in a first container. In some embodiments, a proportion of zinc-solubilizer relative to a total population of bacteria can be higher in a fifth container compared a proportion of zinc-solubilizer relative to a total population of bacteria in a first container. In some embodiments, a proportion of zinc-solubilizer relative to a total population of bacteria can be higher in a sixth container compared a proportion of zinc-solubilizer relative to a total population of bacteria in a first container. In some embodiments, a proportion of zinc-solubilizer relative to a total population of bacteria can be higher in a seventh container compared a proportion of zinc-solubilizer relative to a total population of bacteria in a first container. In some embodiments, a proportion of zinc-solubilizer relative to a total population of bacteria can be higher in an eighth container compared a proportion of zinc-solubilizer relative to a total population of bacteria in a first container. In some embodiments, a proportion of zinc-solubilizer relative to a total population of bacteria can be higher in a ninth container compared a proportion of zinc-solubilizer relative to a total population of bacteria in a first container. In some embodiments, a proportion of zinc-solubilizer relative to a total population of bacteria can be higher in a tenth container compared a proportion of zinc-solubilizer relative to a total population of bacteria in a first container.

### C. Phosphate System

Different steps in digestion of a feedstock may be performed in different reactors as described herein, which may result in (1) a more complete digestion—i.e., more complete breakdown of macromolecules in the feedstock—than other types of bioreactor systems, and/or (2) production of a variety of microbial digestion products having plant growth promoting properties (e.g., ability to enhance or improve soil available phosphate via phosphate solubilization, phosphate uptake by plant tissues, and/or otherwise promote phosphate use efficiency).

In some embodiments, the bioreactor system (e.g., the bioreactor system) comprises an established population of one or more phosphate-solubilizing microbial strains in one or more containers of the system. In some embodiments, a bioreactor system (e.g., one or more containers of a bioreactor system) comprises at least one phosphate-solubilizing microbial strain. In some embodiments, a bioreactor system (e.g., one or more containers of a bioreactor system) comprises an established population of a first phosphate-solubilizing microbial strain and an established population of a second phosphate-solubilizing microbial strain. In some embodiments, the bioreactor system further comprises an established population of a third phosphate-solubilizing microbial strain.

An inoculum of a microbe (e.g., a target microbial strain) may incubate in a reactor (e.g., container) of a bioreactor system described herein. In some embodiments, the inoculum of a microbe may be incubated under conditions that selectively enrich and/or maintain a concentration of the microbial strain in the digestions system. These conditions to selectively enrich and/or maintain a population of the microbial strain may comprise an addition of a selective pressure source (e.g., a phosphate source).

In some cases, the selective pressure can be a phosphate source. In some case, the phosphate source may be a form of insoluble phosphate. Upon addition of a selective pressure (e.g., phosphate source), the inoculation of a phosphate-solubilizing microbial strain may solubilize the insoluble phosphate and create a concentration of high phosphate ion. This environment of high phosphate ion concentration may further enrich a microbial population with a target functionality (e.g., phosphate solubilization).

The inoculum of the microbe may comprise a phosphate-solubilizing microbe that can solubilize insoluble phosphate upon introduction of an insoluble phosphate source (e.g., a selective pressure source, e.g., rock phosphate particles). The phosphate solubilizing microbes can become enriched in the working fluid (e.g., microbial community) of the bioreactor system. Enrichment (e.g., survival) of an inoculum of a microbe may comprise the inoculum of a microbe configured to maintain its initial amount in the environment caused by addition of the selective pressure source. Survival of an inoculum of a microbe may comprise an instance where an amount of the inoculum of the microbe is alive (e.g., maintained) at the end of a retention period of the bioreactor system (e.g., in a reactor or clarifier container). Without wishing to be bound by theory, the inoculum of a microbe may have solubilizing properties for the ion which may allow it to survive in the working fluid. Following addition of the selective pressure, at least a portion of microbes of the microbial consortium may enrich (e.g., grow or increase in number). These microbes of the portion of microbes in the microbial consortium may have phosphate solubilization capacities. Following addition of the selective pressure source, other microbes of the working fluid may reduce in number as the microbes of the microbial consortium are unable to survive (e.g., are susceptible) in the ionic environment. A proportion of the phosphate-solubilizing microbial strain relative to at least a portion of the microbes in a microbial consortium may be maintained in a first container of a bioreactor system. A proportion of the phosphate-solubilizing microbial strain relative to at least a portion of the microbes in a microbial consortium may be maintained in a second, third, fourth, fifth, sixth, seventh, or eighth container of a bioreactor system. A maintained phosphate-solubilizing microbial strain may change its amount in a working fluid of a bioreactor system less than about 0.001%, less than about 0.01%, less than about 0.1%, less than about 0.5%, less than about 1%, less than about 5%, or less than about 10% over a measured time period.

Nutrients (e.g., macronutrients, micronutrients, inorganic nutrients, or any combination thereof) can be present in the bioreactor system to provide an environment for bacterial growth. Macronutrients (phosphorus, carbon, nitrogen, or any combination thereof) may help enrich a population of microbes with a target functionality (e.g., phosphate solubilization). The addition of a selective pressure (e.g., an insoluble phosphate source) with inputs to facilitate microbial growth can allow for enrichment of phosphate solubilizers in the working fluid of a bioreactor system described herein. The phosphate solubilizers can comprise the inoculum of the microbe (e.g., a population of the inoculum of the microbe), phosphate-solubilizing microbes of a microbial consortium, phosphate-solubilizing metabolites produced by the inoculum of the microbe, the phosphate-solubilizing microbes of the microbial consortium, or any combination thereof.

In some embodiments, the selective pressure can enrich a population of phosphate solubilizing microbes (e.g., phosphate solubilizer) in a container and/or containers of a bioreactor system. A population of phosphate solubilizing microbes can comprise an inoculum of a target phosphate solubilizing microbe, phosphate solubilizing microbes of a microbial consortium, phosphate solubilizing metabolites, or any combination thereof.

In some embodiments, as a working fluid flows through a bioreactor system, an absolute number of phosphate-solubilizing microbes may increase. In some embodiments, the absolute number of phosphate-solubilizing microbes may stay substantially the same (e.g., within 80% of a reference number) or decrease, but phosphate-solubilizing activity of microbes in the system may increase. The numbers of phosphate-solubilizing microbes in a fluid may be measured by plating a portion of the fluid on culture plates with a media containing insoluble phosphorus as the only phosphorus source and counting the number of colonies that form halos. In some embodiments, an absolute number of phosphate solubilizers can be higher, the same, or lower in a subsequent container of a bioreactor system compared to an absolute number of phosphate solubilizers in a previous container in a bioreactor system. Despite having similar absolute numbers of phosphate solubilizers in the various containers of a bioreactor system, overall phosphate solubilizing activity of microbes in the system may be stimulated by providing insoluble phosphate as the primary or only source of phosphate in the system.

As working fluid flows through a bioreactor system, a proportion of a particular phosphate-solubilizer or of all phosphate-solubilizers relative to a total population of bacteria may increase, stay substantially the same, or decrease. In some embodiments, a proportion of phosphate-solubilizers relative to a total population of bacteria can be higher, substantially the same, or lower in a subsequent container in a bioreactor system compared a proportion of phosphate-solubilizers relative to a total population of bacteria in a previous container in the system. Despite having similar absolute and/or relative numbers of phosphate solubilizers in the various containers of a bioreactor system, overall phosphate solubilizing activity of microbes in the system may be stimulated by providing insoluble phosphate as the primary or only source of phosphate in the system.

### D. Nitrogen System

Different steps in digestion of a feedstock may be performed in different reactors as described herein, which may result in (1) a more complete digestion—i.e., more complete breakdown of macromolecules in the feedstock—than other types of bioreactor systems, and/or (2) production of a variety of microbial digestion products having plant growth promoting properties (e.g., ability to recruit nitrogen fixers to plant tissues or otherwise promote nitrogen use efficiency).

In some embodiments, the bioreactor system (e.g., the bioreactor system) comprises an established population of one or more nitrogen use efficiency microbial strains in one or more containers of the system. In some embodiments, a bioreactor system (e.g., one or more containers of a bioreactor system) comprises at least one nitrogen use efficiency microbial strain. In some embodiments, a bioreactor system (e.g., one or more containers of a bioreactor system) comprises an established population of a first nitrogen use efficiency microbial strain and an established population of a second nitrogen use efficiency microbial strain. In some embodiments, the bioreactor system further comprises an established population of a third nitrogen use efficiency microbial strain.

An inoculum of a microbe may incubate in a reactor (e.g., container) of a bioreactor system described herein. In some embodiments, the inoculum of a microbe may be incubated under conditions that selectively enrich and/or retain the inoculum of the microbe in the bioreactor system. The nitrogen-use efficiency promoting microbes can comprise the inoculum of the microbe (e.g., a population of a nitrogen-use efficiency promoting microbial strain), nitrogen-use efficiency promoting microbes of a microbial consortium, nitrogen-use efficiency promoting metabolites produced by the inoculum of the microbe and/or the nitrogen-use efficiency promoting microbes of the microbial consortium, or any combination thereof.

The inoculum of the microbe may comprise a nitrogen use efficiency-promoting microbial strain that can be maintained as a population of the microbial strain in a bioreactor system (e.g., one or more containers of a bioreactor system) described herein. Maintenance (e.g., survival) of an inoculum of a microbe may comprise a population of the microbe configured to maintain its initial amount in the environment caused by conditions in a reactor of the bioreactor system. Maintenance (e.g., survival) of an inoculum of a microbe may comprise an instance where an amount of the inoculum of the microbe is alive at the end of a retention period of the bioreactor system (e.g., in a reactor or clarifier container). For example, following initial inoculation, a population of a nitrogen use efficiency-promoting microbial strain may survive incubation in conditions (e.g., nutrients, flow rate, pH, aerobic parameters, or any combination thereof) of a bioreactor system described herein. In some embodiments, at least a portion of microbes of the microbial consortium may enrich (e.g., grow or increase in number). These microbes of the portion of microbes in the microbial consortium may have nitrogen use efficiency capacities. A proportion of the nitrogen use efficiency-promoting microbial strain and nitrogen use efficiency-promoting microbes of the microbial consortium relative to a total population count of bacteria may be maintained in a first container of a bioreactor system. A proportion of the nitrogen use efficiency-promoting microbial strain and nitrogen use efficiency-promoting microbes of the microbial consortium relative to a total population count of bacteria may be maintained in a second, third, fourth, fifth, sixth, seventh, or eighth container of a bioreactor system. A maintained population of a nitrogen use efficiency-promoting microbial strain may change its amount in a working fluid of a bioreactor system less than about 0.001%, less than about 0.01%, less than about 0.1%, less than about 0.5%, less than about 1%, less than about 5%, or less than about 10% over a duration of time. The duration of time may comprise at least about 1 hour, 2 hours, 3 hours, 4 hours, 5 hours, 6 hours, 12 hours, 18 hours, 24 hours, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 3 months, 4 months, 5 months, 6 months, 9 months, 12 months, 18 months, 24 months, 3 years, 4 years, 5 years, 10 years, or greater than 10 years. The duration of time may comprise at most about 10 years, 5 years, 4 years, 3 years, 24 months, 18 months, 12 months, 9 months, 6 months, 5 months, 4 months, 3 months, 8 weeks, 7 weeks, 6 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 24 hours, 18 hours, 12 hours, 6 hours, 5 hours, 4 hours, 3 hours, 2 hours, 1 hour, or less than about 1 hour.

As a working fluid flows through a bioreactor system, an absolute number of nitrogen use efficiency-promoting microbes may increase. In some embodiments, an absolute number of nitrogen use efficiency-promoting microbes can be higher in a second container compared an absolute number of nitrogen use efficiency-promoting microbes in a first container. In some embodiments, an absolute number of nitrogen use efficiency-promoting microbes can be higher in a third container compared an absolute number of nitrogen use efficiency-promoting microbes in a first container. In some embodiments, an absolute number of nitrogen use efficiency-promoting microbes can be higher in a fourth container compared an absolute number of nitrogen use efficiency-promoting microbes in a first container. In some embodiments, an absolute number of nitrogen use efficiency-promoting microbes can be higher in a fifth container compared an absolute number of nitrogen use efficiency-promoting microbes in a first container. In some embodiments, an absolute number of nitrogen use efficiency-promoting microbes can be higher in a sixth container compared an absolute number of nitrogen use efficiency-promoting microbes in a first container. In some embodiments, an absolute number of nitrogen use efficiency-promoting microbes can be higher in a seventh container compared an absolute number of nitrogen use efficiency-promoting microbes in a first container. In some embodiments, an absolute number of nitrogen use efficiency-promoting microbes can be higher in an eighth container compared an absolute number of nitrogen use efficiency-promoting microbes in a first container. In some embodiments, an absolute number of nitrogen use efficiency-promoting microbes can be higher in a ninth container compared an absolute number of nitrogen use efficiency-promoting microbes in a first container. In some embodiments, an absolute number of nitrogen use efficiency-promoting microbes can be higher in a tenth container compared an absolute number of nitrogen use efficiency-promoting microbes in a first container.

As working fluid flows through a bioreactor system, a proportion of nitrogen use efficiency-promoting microbes relative to a total population of bacteria may increase. In some embodiments, a proportion of nitrogen use efficiency-promoting microbes relative to a total population of bacteria can be higher in a second container compared a proportion of nitrogen use efficiency-promoting microbes relative to a total population of bacteria in a first container. In some embodiments, a proportion of nitrogen use efficiency-promoting microbes relative to a total population of bacteria can be higher in a third container compared a proportion of nitrogen use efficiency-promoting microbes relative to a total population of bacteria in a first container. In some embodiments a proportion of nitrogen use efficiency-promoting microbes relative to a total population of bacteria can be higher in a fourth container compared a proportion of nitrogen use efficiency-promoting microbes relative to a total population of bacteria in a first container. In some embodiments, a proportion of nitrogen use efficiency-promoting microbes relative to a total population of bacteria can be higher in a fifth container compared a proportion of nitrogen use efficiency-promoting microbes relative to a total population of bacteria in a first container. In some embodiments, a proportion of nitrogen use efficiency-promoting microbes relative to a total population of bacteria can be higher in a sixth container compared a proportion of nitrogen use efficiency-promoting microbes relative to a total population of bacteria in a first container. In some embodiments, a proportion of nitrogen use efficiency-promoting microbes relative to a total population of bacteria can be higher in a seventh container compared a proportion of nitrogen use efficiency-promoting microbes relative to a total population of bacteria in a first container. In some embodiments, a proportion of nitrogen use efficiency-promoting microbes relative to a total population of bacteria can be higher in an eighth container compared a proportion of nitrogen use efficiency-promoting microbes relative to a total population of bacteria in a first container. In some embodiments, a proportion of nitrogen use efficiency-promoting microbes relative to a total population of bacteria can be higher in a ninth container compared a proportion of nitrogen use efficiency-promoting microbes relative to a total population of bacteria in a first container. In some embodiments, a proportion of nitrogen use efficiency-promoting microbes relative to a total population of bacteria can be higher in a tenth container compared a proportion of nitrogen use efficiency-promoting microbes relative to a total population of bacteria in a first container.

Embodiments of systems and methods described herein produce biostimulant products that may have a multi-modal way of promoting efficient use of nitrogen by plants. Biostimulant products produced by embodiments described herein may be used to promote plant growth by applying the products to plants and/or plant growth media (e.g., soil). One mode of action of products produced in some embodiments is nitrogen-fixing activity of nitrogen fixing bacteria comprised in the products. Another mode of action may be the recruitment of plant associated nitrogen fixers already present in the soil to the roots of plants, thereby increasing nitrogen fixing activity in the root zone and potentially in other plant tissues if the recruited N-fixing microbes become endophytic and can move systemically throughout the plant. Such recruitment may be accomplished by bacterial metabolites present in the biostimulant product and/or by bacterial isolates. Another mode of action may be increases in soil organic nitrogen and mineralization and uptake of organic nitrogen stimulated by microbes and/or microbial metabolites present in the products produced in embodiments described herein.

Biostimulant products described herein may provide plants with N fixation by microbes directly via microbial endophytes and symbionts present in the products, and indirectly via mineralization or decomposition of organically bound N in soil produced by soil N-fixers. Plants may be provided with N through decomposition of organically bound N in the soil.

Embodiments of biostimulant products may also provide plants with the ability to access additional N in soil organic matter. Embodiments of biostimulant products may provide a combination of these strategies to provide better access to biological/organic sources of nitrogen and improve nitrogen use efficiency (NUE).

The following microbe genera can promote nitrogen use efficiency in plants: Kosakonia, Klebsiella, Rahnella, Kluyvera, Enterobacter, Achromobacter, Microbacterium, Gluconobacter, Methylobacterium, Pseudomonas, Pantoea, Azospirillum, Azocarus, Herbaspirillum, Burkholderia, Cyanobacteria, Bacillus, and Paenibacillus. The following microbe species can promote nitrogen use efficiency in plants: Kosakonia sacchari, Klebsiella variicola, Rahnella aquatilis, Kluyvera intermedia, Kosakonia pseusosacchari, Enterobacter spp., Achromobacter marplatensis, Azopirillum lipoferum, Microbacterium murale, Gluconobacter diazotrophicus, Methylobacterium symbioticum, Paenibacillus borealis, Bacillus megaterium (Priestia megaterium), and Paenibacillus sonchi. Embodiments of products described herein may comprise one or more of these microbes. Microbes of these genera may comprise endophytic N fixers (diazotrophs) of monocots.

The inoculum of the microbe, nitrogen use efficiency-promoting microbes of the microbial consortium, nitrogen use efficiency-promoting metabolites, or any combination thereof may be nitrogen use efficiency-promoting microbes in the working fluid of the system and/or in the output product (e.g., base product) of the bioreactor system. These nitrogen use efficiency-promoting microbes may have a nifH gene. A nifH gene can comprise a gene having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or 100% sequence identity to a nucleotide sequence as set forth in SEQ ID NOs: 28 or 29. A nifH gene can comprise a gene that encodes a nitrogenase reductase polypeptide. In some embodiments, the nitrogenase reductase polypeptide has an amino acid sequence that has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or 100% sequence identity to an amino acid sequence encoded by SEQ ID NO: 28 or 29.

Plant associated N-fixers that may be beneficial in biostimulant products described herein may comprise those that can associate with plant roots and other tissues and become endophytic, demonstrate nitrogen fixation activity when associated with roots and other plant tissues, and show consistent increases in plant nitrogen use efficiency, or any combination thereof. Biostimulant products may comprise the following capabilities with respect to nitrogen fixer recruitment: increase the number of associated and/or endophytic N-fixers with plant roots through N-fixer recruitment, nitrogen fixation activity in treated plants, show consistent increases in nitrogen use efficiency, or any combination thereof. Microbial metabolites in biostimulant products may provide for an increased ability of plants to access organically bound nitrogen generated by soil N-fixers and other organic matter, increase soil microbial respiration and biomass, and increase recruitment of beneficial microbes.

In some embodiments, a serialized set of reaction chambers that may be used in a method of producing a biostimulant product are described herein. In some embodiments, conditions within reactor chambers may be established to selectively promote the production of one or more microbes that have a specific desired plant growth promoting effect (e.g., nitrogen use efficiency).

In an aspect, the present disclosure provides a method, comprising (a) transferring an aqueous organic feedstock and an inoculum of a microbe or several microbes that are capable of promoting nitrogen use efficiency in plants into a first container comprising a volume of a first working fluid, wherein the aqueous organic feedstock comprises: (i) a first microbial consortium; and (ii) digestion products produced by digestion of an organic material by microbes in the first microbial consortium; and (b) incubating the inoculum under conditions that promote growth of the nitrogen fixing microbes supplied by the microbial consortia and as well as the specifically introduced microbes, thereby increasing the population of nitrogen fixing microbes and enabling a sustained presence of the specifically introduced microbes.

In some embodiments, the bioreactor system (e.g., the bioreactor system) comprises an established population of one or more nitrogen use efficiency-promoting microbial strains in one or more containers of the system. An “established population” of a particular microbial strain is a population that remains within an operating bioreactor system without replenishing the microbial strain from outside the bioreactor system. In some embodiments, an established population is one that has not been diminished by more than 1, 3, 5, 10, 15, 20, or 25% during continuous operation of the bioreactor system for at least 5, 10, 15, 20, 25, 30, 60, or 90 days without adding a population of the microbial strain to the bioreactor system at a concentration higher than 1, 10, 50, or 100 CFU/ml. In some embodiments, an established population of a microbial strain has been established by making one or more inoculations of the microbial strain into one or more containers of the bioreactor system. In some embodiments, an established population is a population that is derived from a population that was inoculated into the system at least 10, 30, 60, or 90 days previous.

In some embodiments, a bioreactor system (e.g., one or more containers of a bioreactor system) comprises at least one microbial strain. In some embodiments, a bioreactor system (e.g., one or more containers of a bioreactor system) comprises at least one nitrogen use efficiency-promoting microbial strain. In some embodiments, a bioreactor system (e.g., one or more containers of a bioreactor system) comprises an established population of a first nitrogen use efficiency-promoting microbial strain and an established population of a second nitrogen use efficiency-promoting microbial strain. In some embodiments, the bioreactor system further comprises an established population of a third nitrogen use efficiency-promoting microbial strain. The established populations of the respective microbial strains may be established in individual or combined inoculations into the bioreactor system. An individual inoculation may comprise one inoculum of a microbial strain (e.g., microbe). A combined inoculation may comprise an inoculum comprising at least two microbial strains. The combined inoculation may comprise the same isolated microbial strains. The combined inoculation may comprise an isolated microbial strain and non-isolated microbial strain. The combined inoculation may comprise two or more isolated microbial strains.

In some embodiments, nitrogen use efficiency promotion may comprise increasing nitrogen fixation, promoting nitrogen fixation in the root and other tissues of plants, recruiting nitrogen fixers to the roots of plants, promoting soil organic nitrogen content and mineralization and uptake of organic nitrogen from soil, or any combination thereof. In some embodiments, the microbe is capable of nitrogen fixation, promoting nitrogen fixation in the root and other tissues of plants, recruiting nitrogen fixers to the roots of plants, promoting soil organic nitrogen content and mineralization and uptake of organic nitrogen from soil, or any combination thereof. In some embodiments, the conditions promote growth of one or more microbes in the first microbial consortium that are capable of promoting plant growth, nitrogen fixation, nitrogen use efficiency, or recruitment of nitrogen fixers to the roots of plants, or of generating metabolites capable of promoting plant growth, nitrogen fixation, nitrogen use efficiency, enhancing organic nitrogen in the soil and mineralization and plant uptake of organic nitrogen, or recruitment of nitrogen fixing microbes to the roots of plants. In some embodiments, during the incubating the microbe or one or more microbes in the first microbial consortium, metabolites are produced capable of promoting plant growth and nitrogen use efficiency. In some embodiments, the incubating increases a population of one or more microbes in the microbial consortium capable of promoting plant growth. In some embodiments, the aqueous organic feedstock further comprises an inorganic substrate. In some embodiments, the first microbial consortium further comprises microbes derived from the inorganic substrate. In some embodiments, the inorganic substrate comprises rock phosphate. In some embodiments, the microbe or microbes are of the species Paenibacillus borealis, Bacillus megaterium, or Paenibacillus sonchi. In some embodiments, the microbe is the Paenibacillus borealis strain deposited under ATCC Accession No. PTA-127654 (MS3907), the Bacillus megaterium (or Priestia megaterium) strain deposited under ATCC Accession No. PTA-127653 (MS3900), the Paenibacillus sonchi strain deposited under ATCC Accession No. PTA-127655 (MS4921), or the Bacillus megaterium strain deposited under ATCC Accession No. PTA-127652 (MS2748). In some embodiments, the first working fluid comprises (a) a second microbial consortium derived from the aqueous organic feedstock, and/or (b) digestion products produced by digestion of substances present in the organic feedstock by the first microbial consortium and the microbe(s). In some embodiments, the method further comprises transferring a portion of the first working fluid into a second container. The second container can comprise a second working fluid. The method may further comprise incubating the second working fluid in the second container. In some embodiments, the method further comprises transferring a portion of the first working fluid into a second container comprising a second working fluid and incubating the second working fluid in the second container. The second working fluid may comprise a third microbial consortium and the third microbial consortium may be derived from the first working fluid. The second working fluid may comprise digestion products produced by digestion of substances present in the first working fluid by the third microbial consortium, the microbial strain, or any combination thereof. In some embodiments, the second working fluid comprises (a) a third microbial consortium derived from the first working fluid, and (b) digestion products produced by digestion of substances present in the first working fluid by the third microbial consortium and the microbe. In some embodiments, the amount of the aqueous organic feedstock transferred into the first container over a time period is equal to the amount of the first working fluid transferred into the second container over the same time period. In some embodiments, the amount of the aqueous organic feedstock transferred into the first container over a time period is not equal to the amount of the first working fluid transferred into the second container over the same time period. In some embodiments, the volume of the first working fluid in the first container is maintained constant. In some embodiments, the volume of the first working fluid in the first container is not maintained constant. In some embodiments, transferring the aqueous organic feedstock into the first container comprises continuously flowing the aqueous organic feedstock into the first container at a first flow rate, transferring the portion of the first working fluid into the second container comprises continuously flowing the portion of the first working fluid into the second container at a second flow rate, and the first flow rate and the second flow rate are equal. In some embodiments, the method further comprises transferring a portion of the second working fluid to a third container comprising a third working fluid and incubating the third working fluid in the third container. In some embodiments, the method further comprises transferring a portion of the third working fluid into a fourth container comprising a fourth working fluid and incubating the fourth working fluid in the fourth container. In some embodiments, the first working fluid, the second working fluid, the third working fluid, and the fourth working fluid are maintained at constant volumes. In some embodiments, a plant growth promoting product made by the method described herein may promote nitrogen use efficiency in a plant. In some embodiments, a method of promoting nitrogen use efficiency of a plant comprises contacting the plant and/or a medium in which the plant is growing with the product.

### VIII. Microbial Consortia and Microbial Isolates

Certain microorganisms of the present disclosure have all of the identifying characteristics of the deposited strains and, in particular, the identifying characteristics of being able to promote plant growth and/or yield as described herein. In particular, the certain microorganisms of the present disclosure can refer to the deposited microorganisms as described herein, and strains derived therefrom.

In some embodiments, the microbial consortia may be characterized by population analysis. The population analysis may comprise a community analysis, determination of the core community, and computation of a microbial-community distance matrix. In some embodiments, the microbial consortia may be characterized in batches. Characterization of the microbial consortia may be measured in 1, 2, 3, 4, 5, 6, 7, 8 or more batches. From the population analysis, the most abundant species of the microbial consortia may be determined. In some embodiments, the most abundant species of a microbial consortia comprise the top 2, 3, 4, 5, 10, 15, or 20 species. In some embodiments, different reactors may have microbial consortia with different species being the most abundant.

In some embodiments, a first microbial consortium may be established in a mixing chamber, in which various inputs may be mixed into a homogenous aqueous mixture to be input into a digestion reactor. In some embodiments, a digestions system described herein may comprise one or more mixing chambers in which a microbial consortium may be established. A mixing chamber may be fed an input or a series of inputs (e.g., target microbial strain, aqueous feedstock, carbon source, nitrogen source, nutrients, water, or any combination thereof) which may be mixed prior to being inputted into a first container (e.g., a first reactor). The first microbial consortium may be derived from microbes originally present in one or more digestion substrates and/or from other inputs into the mixing chamber. A first microbial consortium may be derived from inputs to a reactor in a bioreactor system described herein. The inputs may be from a whole broth (WB) from another bioreactor system (e.g., water-based phosphate solubilizing technology (PwST), phosphate solubilizing technology (PST), zinc solubilizing technology (ZST)). Inputs may comprise but are not limited to, manure, digestion products of manure, microbes derived from manure, rock phosphate, algae, digestion products of algae, or microbes derived from algae. In some embodiments, a second microbial consortium is established in a first reactor. The second microbial consortium may be derived from microbes within the mixing chamber. In some embodiments, a third microbial consortium is established in a second reactor. The third microbial consortium may be derived from the first working fluid present in the first reactor and transferred to the second reactor. In some embodiments, a fourth microbial consortium is established in a third reactor. The fourth microbial consortium may be derived from the second working fluid present in the second reactor and transferred to the third reactor. In some embodiments, a fifth microbial consortium is established in a fourth reactor. The fifth microbial consortium may be derived from the fourth working fluid present in the fourth reactor and transferred to the fifth reactor. A microbial consortium can be present in any reactor of a bioreactor system described herein. A microbial consortium can be derived from the working fluid of a reactor of a bioreactor system described herein. A first microbial consortium may be derived from inputs to a first reactor and may be present in a base product of a bioreactor system. A first microbial consortium may be present in a first reactor, a second reactor, a third reactor, or a clarifier. Without wishing to be bound by theory, a first microbial consortium in a working fluid may shift its microbial population and form a second microbial consortium. A second microbial consortium may be present in a first reactor, a second reactor, a third reactor, or a clarifier. Without wishing to be bound by theory, a second microbial consortium in a working fluid may shift its microbial population and form a third microbial consortium. A third microbial consortium may be present in a first reactor, a second reactor, a third reactor, or a clarifier. Without wishing to be bound by theory, a third microbial consortium in a working fluid may shift its microbial population and form a fourth microbial consortium. A fourth microbial consortium may be present in a first reactor, a second reactor, a third reactor, or a clarifier. Without wishing to be bound by theory, a fourth microbial consortium in a working fluid may shift its microbial population and form a fifth microbial consortium. A fifth microbial consortium may be present in a first reactor, a second reactor, a third reactor, a fourth reactor, or a clarifier. Microbial consortia of the bioreactor system described herein may develop shifts in microbial communities based on conditions (e.g., nutrients, retention time, flow rate, pH, oxygen content, digestion products) of the reactors of the system and the working fluid. Without wishing to be bound by theory, as a working fluid incubates in a reactor of a bioreactor system (e.g., one or more containers of a bioreactor system) described herein, microbes of a microbial consortium may become enriched, be maintained, or die which can shift a population of microbes in a microbial community in a working fluid and establish a new microbial consortium.

A portion of a first working fluid may be transferred to a second container of a serialized assembly of containers of a bioreactor system described herein. The working fluid of the second container may comprise a second working fluid. A portion of a second working fluid may be transferred to a third container of a serialized assembly of containers of a bioreactor system described herein. The working fluid of the third container may comprise a third working fluid. A portion of a third working fluid may be transferred to a fourth container of a serialized assembly of containers of a bioreactor system described herein. The working fluid of the fourth container may comprise a fourth working fluid. A portion of a fourth working fluid may be transferred to a fifth container of a serialized assembly of containers of a bioreactor system described herein. The working fluid of the fifth container may comprise a fifth working fluid. A portion of a fifth working fluid may be transferred to a sixth container of a serialized assembly of containers of a bioreactor system described herein. The working fluid of the sixth container may comprise a sixth working fluid.

The working fluid of a container in the bioreactor system may incubate in the container. A flow rate of the bioreactor system may increase or decrease a volume of working fluid, or the volume of the working fluid may stay the same. Adjusting a flow rate of the system may result in a longer or shorter hydraulic retention time for the system, while keeping the volume of working fluid in each container constant. The working fluid of a first, second, third, fourth, fifth, or sixth container may increase in volume over a time period. The working fluid of a first, second, third, fourth, fifth, or sixth container may decrease in volume over a time period. The working fluid of a first, second, third, fourth, fifth, or sixth container may not increase or decrease in volume over a time period. The volume of working fluid in each of the containers of a bioreactor system may be the same. The volume of working fluid in each of the containers of a bioreactor system may be different. The volumes of the first working fluid, second working fluid, third working fluid, fourth working fluid, fifth working fluid, and/or sixth working fluid may be constant (e.g., unchanging over a time period). A constant volume may comprise a volume that does not increase or decrease over about 1 minute, about 5 minutes, about 10 minutes, about 30 minutes, about 1 hour, about 5 hours, about 12 hours, about 1 day, about 2 days, about 3 days, about 4 days, about 5 days, about 6 days, or about 1 week.

As working fluid flows from each container of bioreactor system, the working fluid may comprise a flow rate. A first flow rate may comprise a flow rate of an aqueous feedstock inputted into a first container from a source outside the bioreactor system. A second flow rate may comprise a flow rate of a working fluid from a first container into a second container. A third flow rate may comprise a flow rate of a working fluid from a second container into a third container. A fourth flow rate may comprise a flow rate of a working fluid from a third container into a fourth container. A fifth flow rate may comprise a flow rate of a working fluid from a fourth container into a fifth container. A flow rate (e.g., first flow rate, second flow rate, third flow rate, fourth flow rate, fifth flow rate) may be at least about 0.5 gallons/min, 1 gallon/min, 5 gallons/min, 10 gallons/min, 15 gallons/min, 20 gallons/min, 25 gallons/min, 30 gallons/min, 40 gallons/min, 50 gallons/min, 100 gallons/min, 200 gallons/min, 300 gallons/min, 400 gallons/min, 500 gallons/min, 1,000 gallons/min, or 10,000 gallons/min. A flow rate (e.g., first flow rate, second flow rate, third flow rate, fourth flow rate, fifth flow rate) may be at most about 10,000 gallons/min 1,000 gallons/min, 500 gallons/min, 400 gallons/min, 300 gallons/min, 200 gallons/min, 100 gallons/min, 50 gallons/min, 40 gallons/min, 30 gallons/min, 25 gallons/min, 20 gallons/min, 15 gallons/min, 10 gallons/min, 5 gallons/min, 1 gallon/min, 0.5 gallons/min, or less than about 0.5 gallons/min.

A microbial consortium can comprise a population of microbes. The population of microbes can be generated from an input to the bioreactor system. An aqueous feedstock inputted into the bioreactor system may comprise a microbial consortium. Incubation of a working fluid in the bioreactor system may promote the growth of microbes within a microbial consortium (e.g., a first microbial consortium, a second microbial consortium, a third microbial consortium, a fourth microbial consortium, a fifth microbial consortium, a sixth microbial consortium). The microbes of a microbial consortium or at least a portion of microbes within the microbial consortium may have a desired plant growth promotion property. The plant growth promotion property may comprise shoot biomass, root biomass, nutrient uptake, crop yield, zinc solubilization, phosphate solubilization, deaminase activity, acid production, leaf area, chlorophyll content, photosynthetic activity, or total biomass.

Reactors of a bioreactor system may be fluidly connected. A portion of a working fluid in a first container may be transferred to a fluidly connected second container. A portion of a working fluid in a second container may be transferred to a fluidly connected third container. A portion of a working fluid in a third container may be transferred to a fluidly connected fourth container. A portion of a working fluid in a fourth container may be transferred to a fluidly connected fifth container. A portion of a working fluid in a fifth container may be transferred to a fluidly connected sixth container. Additional fluidly connected containers may be arranged in a similar way. In some embodiments, a transfer of working fluid between containers of a bioreactor system described herein may be continuous. A continuous flow of working fluid may comprise a flow of working fluid that does not stop or a flow of working fluid that stops for less than about 5 seconds, less than about 4 seconds, less than about 3 seconds, less than about 2 seconds, less than about 1 second, less than about 0.5 seconds, or less than about 0.1 seconds. A continuous flow of working fluid within a bioreactor system described herein (e.g., between containers of a bioreactor system) may have a first flow rate. A continuous flow of working fluid within a bioreactor system described herein (e.g., between containers of a bioreactor system) may have a second flow rate. In some embodiments, the first flow rate and the second flow rate are equal. A first flow rate may comprise a flow rate of fluid transferred from a source outside the bioreactor system into a first container. A second flow rate may comprise a rate of fluid flow from a first container to a second container. In some embodiments, an amount of working fluid and/or feedstock (e.g., aqueous organic feedstock) transferred into the first container over a time period is equal to an amount of working fluid and/or feedstock (e.g., aqueous organic feedstock) transferred into a second fluidly connected container over the same time period. In some embodiments, the first flow rate and the second flow rate are different. In some embodiments, a first flow rate may be faster than a second flow rate. In some embodiments, a first flow rate may be slower than a second flow rate. For example, flow rates may be different if an input stream into a first reactor is faster or slower than a stream of working fluid transferred from the first reactor to a second reactor. This difference in flow rates may result in different volumes of working fluid in containers of a bioreactor system.

In some embodiments, a first container of a bioreactor system comprises a constant volume. In some embodiments, a volume of a first container of a bioreactor system is different over time. In some embodiments, the flow rate between containers of the bioreactor system may maintain a constant volume in each container. A first container, a second container, a third container, a fourth container, a fifth container, and/or a sixth container may be maintained at a constant volume. A constant volume may be maintained by a continuous flow of fluid through a bioreactor system described herein. For example, similar flow rates of working fluid being transferred between containers of a bioreactor system may result in constant volumes of working fluid in the containers. This continuous flow of the bioreactor system may help enrich a microbial population with a targeted functionality (e.g., zinc solubilization, phosphate solubilization, nitrogen use efficiency) by not overflowing or depleting a working fluid in a container of the system.

In some embodiments, a bioreactor system may be inoculated with an inoculum of a microbe (e.g., an inoculum of a microbial strain). The inoculum of a microbial strain may be an isolated microbe. An isolated microbe can comprise a microbe grown or enriched outside of a natural environment (e.g., in a culture medium or a streak plate method). In some embodiments, the inoculum of a microbe may comprise a mixture of two or more isolated microbes. The inoculum of a microbe may comprise a mixture of at least about 2, at least about 3, at least about 4, at least about 5, at least about 6, at least about 7, at least about 8, or more isolated microbes.

In some cases, a bioreactor system may not be reinoculated with an isolate (e.g., microbial strain) or combination of isolates following a first inoculation. Reinoculation of a bioreactor system may comprise providing a microbial strain following a previous inoculation. Reinoculation of a bioreactor system may comprise introducing a target isolate (e.g., microbial strain) in a container of the bioreactor system at a time point during operating of the bioreactor system. In some cases, a bioreactor system may be reinoculated with an isolate or combination of isolates at least every 20 days, at least every 50 days, at least every 100 days, at least every 200 days, at least every 300 days, at least every 400 days, at least every 500 days, or more. In some cases, a bioreactor system may be inoculated with an isolate or combination of isolates on day 1 of a digestion process and reinoculated 1, 2, 3, 4, 5, or more times after day 1 of the digestion process. In some cases, a bioreactor system may be reinoculated with a microbial strain described herein after operating the bioreactor system for a time period. For example, a bioreactor system may be reinoculated with a microbial strain described herein after operating the bioreactor system for a duration of time of at least about 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 3 months, 4 months, 5 months, 6 months, 9 months, 12 months, 18 months, 24 months, 3 years, 4 years, 5 years, or 10 years. For example, a digestion system may be reinoculated with a microbial strain described herein after operating the digestion system for a duration of time of at most about 10 years 5 years, 4 years, 3 years, 24 months, 18 months, 12 months, 9 months, 6 months, 5 months, 4 months, 3 months, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, or 1 day.

In some embodiments, the inoculation establishes an initial population of the target isolate having a concentration of at least about 1×10² CFU/ml, at least about 0.5×10³ CFU/ml, at least about 1×10³ CFU/ml, at least about 0.5×10⁴ CFU/ml, at least about 1×10⁴ CFU/ml, at least about 0.5×10⁵ CFU/ml, at least about 1×10⁵ CFU/ml, at least about 0.5×10⁶ CFU/ml, at least about 1×10⁶ CFU/ml, at least about 1×10⁷ CFU/ml, at least about 1×10⁸ CFU/ml, or greater than about 1×10⁸ CFU/ml. In some embodiments, the inoculation establishes an initial population of the target isolate having a concentration of at most about at least about 1×10⁸ CFU/ml, at least about 1×10⁷ CFU/ml, at least about 1×10⁶ CFU/ml, at least about 0.5×10⁶ CFU/ml, at least about 1×10⁵ CFU/ml, at least about 0.5×10⁵ CFU/ml, at least about 1×10⁴ CFU/ml, at least about 0.5×10⁴ CFU/ml, at least about 1×10³ CFU/ml, at least about 0.5×10³ CFU/ml, 1×10² CFU/ml, or less than about 1×10² CFU/ml.

In some embodiments, the population of the microbial strain (e.g., concentration of the microbial strain) is maintained (e.g., retained) by at least about 0.00001%, at least about 0.0001%, at least about 0.001%, at least about 0.01%, at least about 0.1%, at least about 0.5%, at least about 1%, at least about 5%, at least 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% from the concentration of the microbial strain added into a container (e.g., a first container) of the bioreactor system. Biosolids (e.g., floc) may comprise small particles from a working fluid of a bioreactor system. The biosolids (e.g., floc) may accumulate in a clarifier container over time and separate from a supernatant (e.g., base product). In some embodiments, the population of the microbial strain may be retained in the floc (e.g., biosolids) of the bioreactor system. In some embodiments, a majority (95%, 96%, 97%, 98%, 99%, 99.5%, 99.6%, 99.7%, 99.8%, or 99.9%) of the inoculum of the microbe may be retained in the floc (e.g., biosolids) of the bioreactor system. Floc may be generated at any point during operating of a bioreactor system as described herein. For example, floc may be generated in a reactor of the bioreactor system (e.g., a first container, a second container, a third container, a fourth container, a fifth container, a sixth container, or any container of the system). For example, floc may be generated in a clarifier container of a bioreactor system. In some embodiments, floc may comprise at least a portion of zinc-solubilizing microbes generated in a bioreactor system described herein. In some embodiments, floc may comprise at least a portion of phosphate-solubilizing microbes generated in a bioreactor system described herein. In some embodiments, floc may comprise at least a portion of nitrogen use efficiency-promoting microbes generated in a bioreactor system described herein.

In some embodiments, there may be at least about 1 log CFU/ml, at least about 2 logs CFU/ml, at least about 3 logs CFU/ml, at least about 4 logs CFU/ml, at least about 5 logs CFU/ml, or greater than about 5 logs CFU/ml increase in a concentration of the population of the microbial strain after inoculation in a container (e.g., first container) of the bioreactor system described herein. In some embodiments, there may be at most about 5 logs CFU/ml, at most about 4 logs CFU/ml, at most about 3 logs CFU/ml, at most about 2 logs CFU/ml, at most about 1 log CFU/ml, or less than about 1 log CFU/ml increase in a concentration of the population of the microbial strain after inoculation in a container (e.g., first container) of the bioreactor system described herein. In some embodiments, there may be from about 1 log CFU/ml to about 8 logs CFU/ml increase in a concentration of the population of the microbial strain after inoculation in a container (e.g., first container) of the bioreactor system described herein. In some embodiments, there may be from about 1 log CFU/ml to about 2 logs CFU/ml, about 1 log CFU/ml to about 3 logs CFU/ml, about 1 log CFU/ml to about 4 logs CFU/ml, about 1 log CFU/ml to about 5 logs CFU/ml, about 1 log CFU/ml to about 6 logs CFU/ml, about 1 log CFU/ml to about 7 logs CFU/ml, about 1 log CFU/ml to about 8 logs CFU/ml, about 2 logs CFU/ml to about 3 logs CFU/ml, about 2 logs CFU/ml to about 4 logs CFU/ml, about 2 logs CFU/ml to about 5 logs CFU/ml, about 2 logs CFU/ml to about 6 logs CFU/ml, about 2 logs CFU/ml to about 7 logs CFU/ml, about 2 logs CFU/ml to about 8 logs CFU/ml, about 3 logs CFU/ml to about 4 logs CFU/ml, about 3 logs CFU/ml to about 5 logs CFU/ml, about 3 logs CFU/ml to about 6 logs CFU/ml, about 3 logs CFU/ml to about 7 logs CFU/ml, about 3 logs CFU/ml to about 8 logs CFU/ml, about 4 logs CFU/ml to about 5 logs CFU/ml, about 4 logs CFU/ml to about 6 logs CFU/ml, about 4 logs CFU/ml to about 7 logs CFU/ml, about 4 logs CFU/ml to about 8 logs CFU/ml, about 5 logs CFU/ml to about 6 logs CFU/ml, about 5 logs CFU/ml to about 7 logs CFU/ml, about 5 logs CFU/ml to about 8 logs CFU/ml, about 6 logs CFU/ml to about 7 logs CFU/ml, about 6 logs CFU/ml to about 8 logs CFU/ml, or about 7 logs CFU/ml to about 8 logs CFU/ml increase in a concentration of the population of the microbial strain after inoculation in a container (e.g., first container) of the bioreactor system described herein.

In some embodiments, there may be at least about 1 log CFU/ml, at least about 2 logs CFU/ml, at least about 3 logs CFU/ml, at least about 4 logs CFU/ml, at least about 5 logs CFU/ml, or greater than about 5 logs CFU/ml decrease in a concentration of the population of the microbial strain after inoculation in a container (e.g., first container) of the bioreactor system described herein. In some embodiments, there may be at most about 5 logs CFU/ml, at most about 4 logs CFU/ml, at most about 3 logs CFU/ml, at most about 2 logs CFU/ml, at most about 1 log CFU/ml, or less than about 1 log CFU/ml decrease in a concentration of the population of the microbial strain after inoculation in a container (e.g., first container) of the bioreactor system described herein. In some embodiments, there may be from about 1 log CFU/ml to about 8 logs CFU/ml decrease in a concentration of the population of the microbial strain after inoculation in a container (e.g., first container) of the bioreactor system described herein. In some embodiments, there may be from about 1 log CFU/ml to about 2 logs CFU/ml, about 1 log CFU/ml to about 3 logs CFU/ml, about 1 log CFU/ml to about 4 logs CFU/ml, about 1 log CFU/ml to about 5 logs CFU/ml, about 1 log CFU/ml to about 6 logs CFU/ml, about 1 log CFU/ml to about 7 logs CFU/ml, about 1 log CFU/ml to about 8 logs CFU/ml, about 2 logs CFU/ml to about 3 logs CFU/ml, about 2 logs CFU/ml to about 4 logs CFU/ml, about 2 logs CFU/ml to about 5 logs CFU/ml, about 2 logs CFU/ml to about 6 logs CFU/ml, about 2 logs CFU/ml to about 7 logs CFU/ml, about 2 logs CFU/ml to about 8 logs CFU/ml, about 3 logs CFU/ml to about 4 logs CFU/ml, about 3 logs CFU/ml to about 5 logs CFU/ml, about 3 logs CFU/ml to about 6 logs CFU/ml, about 3 logs CFU/ml to about 7 logs CFU/ml, about 3 logs CFU/ml to about 8 logs CFU/ml, about 4 logs CFU/ml to about 5 logs CFU/ml, about 4 logs CFU/ml to about 6 logs CFU/ml, about 4 logs CFU/ml to about 7 logs CFU/ml, about 4 logs CFU/ml to about 8 logs CFU/ml, about 5 logs CFU/ml to about 6 logs CFU/ml, about 5 logs CFU/ml to about 7 logs CFU/ml, about 5 logs CFU/ml to about 8 logs CFU/ml, about 6 logs CFU/ml to about 7 logs CFU/ml, about 6 logs CFU/ml to about 8 logs CFU/ml, or about 7 logs CFU/ml to about 8 logs CFU/ml decrease in a concentration of the population of the microbial strain after inoculation in a container (e.g., first container) of the bioreactor system described herein.

### A. Zinc Solubilizer Microbial Isolates

Zinc is an essential micronutrient for plant growth and development. Zinc acts as an anti-oxidant in plants and is important in carbohydrate metabolism, auxin metabolism, and other plant growth promoting properties. A small amount of zinc may be present in soil as soluble zinc, with the rest present as insoluble minerals and other complexes. Zinc solubilizing microorganisms can solubilize zinc through acidification, excretion of organic acids, vitamins, phytohormones, and amino acids, oxido-reductive systems, production of chelating agents, proton extrusion, or other mechanisms of bioaugmentation. In some embodiments, disclosed herein are zinc solubilizing microbial strains, including compositions that comprise such strains and methods of using such strains to promote plant growth.

In some embodiments, desirable microbes may have zinc-solubilizing properties (e.g., enhancing bioavailability of zinc, increasing zinc uptake, or any combination thereof). The other desirable microbes may be of the bacterial species Bacillus safensis, Bacillus amyloliquefaciens, Bacillus megaterium, Pseudomonas spp., Agrobacterium spp., Bacillus circulans, Burkholderia lata, Lysinibacillus sp., Burkholderia latens, Bacillus cereus, B. aryabhattai, Pseudomonas taiwenensis, Acinetobacter sp., Paenibacillus sonchi, Chitinophaga spp., Gluconobacter spp., Klebsiella spp., Serratia spp., Thiobacillus spp., or any combination thereof.

In some embodiments, operating a bioreactor system (e.g., one or more containers of a bioreactor system) disclosed herein provides a synergistic effect in the production of a biostimulant composition. In some embodiments, a bioreactor system that comprises an established and/or stable population of a zinc-solubilizing microbial strain (e.g., one or more of the zinc-solubilizing strains disclosed herein and/or related strains) produces a biostimulant having a greater zinc-solubilizing effect than could be obtained from an otherwise similar bioreactor system that does not have the established population of the zinc-solubilizing strain. The zinc-solubilizing effect of the biostimulant produced according to methods disclosed herein may be greater than a biostimulant produced in an otherwise similar system lacking the established population of the zinc-solubilizing strain, even if the latter biostimulant has one or more of the zinc-solubilizing strains added to it after it is produced. For example, a biostimulant composition produced according to the methods disclosed herein may have greater capacity to increase zinc uptake compared to a biostimulant composition produced by an otherwise similar system lacking the established population of the zinc-solubilizing strain. For example, a biostimulant composition produced according to the methods disclosed herein may have greater zinc-solubilizing effects compared to a biostimulant composition produced by an otherwise similar system lacking the established population of the zinc-solubilizing strain. For example, a biostimulant composition produced according to the methods disclosed herein may have greater capacity to increase soil organically complexed zinc and mineralization and uptake of zinc from organic complexes compared to that of a biostimulant composition produced by an otherwise similar system lacking the established population of the zinc-solubilizing strain. Without being bound by theory, the presence of the target zinc-solubilizing strain population in the bioreactor system may promote production of zinc-solubilizing digestion products in the bioreactor system, which are present in the product.

In some embodiments, the microbial strain is from a Bacillus genus. In some embodiments, the microbial strain is from a Bacillus safensis species. In some embodiments, the microbial strain is from a bacterial species other than Bacillus safensis. In some embodiments, the Bacillus safensis strain is deposited under ATCC Accession No. PTA-127681 or an isolated clone thereof. In some embodiments, the 16S rRNA gene of the microbial strain comprises the nucleotide sequence of SEQ ID NO.: 1. In some embodiments, the microbial strain comprises a 16S rRNA gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to the nucleotide sequences as set forth in SEQ ID NO.: 1. In some embodiments, the 16S rRNA gene of the microbial strain comprises a nucleotide sequence that exhibits at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to the nucleotide sequences as set forth in SEQ ID NO.: 1. In some embodiments, the gyrB gene of the microbial strain comprises the nucleotide sequence of SEQ ID NO: 4. In some embodiments, the microbial strain comprises a gyrB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% sequence identity to a nucleotide sequence as set forth in SEQ ID NO: 4. In some embodiments, the rpoB gene of the microbial strain comprises the nucleotide sequence of SEQ ID NO: 7. In some embodiments, the microbial strain comprises a rpoB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% sequence identity to a nucleotide sequence as set forth in SEQ ID NO: 7. Some embodiments provide a genus of plant growth-promoting microorganisms comprising any of the DNA sequences described herein and which enhances the health, growth and/or yield of a plant, as described herein.

In some embodiments, the microbial strain is from a Bacillus megaterium species. In some embodiments, the microbial strain is from a bacterial species other than Bacillus megaterium. In some embodiments, the Bacillus megaterium strain is deposited under ATCC Accession No. PTA-127683 or an isolated clone thereof. In some embodiments, the 16S rRNA gene of the microbial strain comprises the nucleotide sequence of SEQ ID NO.: 2. In some embodiments, the microbial strain comprises a 16S rRNA gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to the nucleotide sequences as set forth in SEQ ID NO.: 2. In some embodiments, the 16S rRNA gene of the microbial strain comprises a nucleotide sequence that exhibits at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to the nucleotide sequences as set forth in SEQ ID NO.: 2. In some embodiments, the gyrB gene of the microbial strain comprises the nucleotide sequence of SEQ ID NO: 5. In some embodiments, the microbial strain comprises a gyrB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% sequence identity to a nucleotide sequence as set forth in SEQ ID NO: 5. In some embodiments, the rpoB gene of the microbial strain comprises the nucleotide sequence of SEQ ID NO: 8. In some embodiments, the microbial strain comprises a rpoB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% sequence identity to a nucleotide sequence as set forth in SEQ ID NO: 8. Some embodiments provide a genus of plant growth-promoting microorganisms comprising any of the DNA sequences described herein and which enhances the health, growth and/or yield of a plant, as described herein.

In some embodiments, the microbial strain is from a Bacillus megaterium species. In some embodiments, the microbial strain is from a bacterial species other than Bacillus megaterium. In some embodiments, the Bacillus megaterium strain is deposited under ATCC Accession No. PTA-127682 or an isolated clone thereof. In some embodiments, the 16S rRNA gene of the microbial strain comprises the nucleotide sequence of SEQ ID NO.: 3. In some embodiments, the microbial strain comprises a 16S rRNA gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to the nucleotide sequences as set forth in SEQ ID NO.: 3. In some embodiments, the 16S rRNA gene of the microbial strain comprises a nucleotide sequence that exhibits at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to the nucleotide sequences as set forth in SEQ ID NO.: 3. In some embodiments, the gyrB gene of the microbial strain comprises the nucleotide sequence of SEQ ID NO: 6. In some embodiments, the microbial strain comprises a gyrB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% sequence identity to a nucleotide sequence as set forth in SEQ ID NO: 6. In some embodiments, the rpoB gene of the microbial strain comprises the nucleotide sequence of SEQ ID NO: 9. In some embodiments, the microbial strain comprises a rpoB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% sequence identity to a nucleotide sequence as set forth in SEQ ID NO: 9. Some embodiments provide a genus of plant growth-promoting microorganisms comprising any of the DNA sequences described herein and which enhances the health, growth and/or yield of a plant, as described herein.

TABLE 1
Exemplary 16S rRNA Sequences of Bacterial Isolates

| SEQ ID | | |
|---|---|---|
| NO. | Strain | Sequence |
| 1 | MS4666 | GACGAACGCTGGCGGCGTGCCTAATACATGCAAGTCGAGCGGACAGAAGGGAGCTTGCT |
| | | CCCGGATGTTAGCGGCGGACGGGTGAGTAACACGTGGGTAACCTGCCTGTAAGACTGGG |
| | | ATAACTCCGGGAAACCGGAGCTAATACCGGATAGTTCCTTGAACCGCATGGTTCAAGGA |
| | | TGAAAGACGGTTTCGGCTGTCACTTACAGATGGACCCGCGGCGCATTAGCTAGTTGGTG |
| | | GGGTAATGGCTCACCAAGGCGACGATGCGTAGCCGACCTGAGAGGGTGATCGGCCACAC |
| | | TGGGACTGAGACACGGCCCAGACTCCTACGGGAGGCAGCAGTAGGGAATCTTCCGCAAT |
| | | GGACGAAAGTCTGACGGAGCAACGCCGCGTGAGTGATGAAGGTTTTCGGATCGTAAAGC |
| | | TCTGTTGTTAGGGAAGAACAAGTGCGAGAGTAACTGCTCGCACCTTGACGGTACCTAAC |
| | | CAGAAAGCCACGGCTAACTACGTGCCAGCAGCCGCGGTAATACGTAGGTGGCAAGCGTT |
| | | GTCCGGAATTATTGGGCGTAAAGGGCTCGCAGGCGGTTTCTTAAGTCTGATGTGAAAGC |
| | | CCCCGGCTCAACCGGGGAGGGTCATTGGAAACTGGGAAACTTGAGTGCAGAAGAGGAGA |
| | | GTGGAATTCCACGTGTAGCGGTGAAATGCGTAGAGATGTGGAGGAACACCAGTGGCGAA |
| | | GGCGACTCTCTGGTCTGTAACTGACGCTGAGGAGCGAAAGCGTGGGGAGCGAACAGGAT |
| | | TAGATACCCTGGTAGTCCACGCCGTAAACGATGAGTGCTAAGTGTTAGGGGGTTTCCGC |
| | | CCCTTAGTGCTGCAGCTAACGCATTAAGCACTCCGCCTGGGGAGTACGGTCGCAAGACT |
| | | GAAACTCAAAGGAATTGACGGGGGCCCGCACAAGCGGTGGAGCATGTGGTTTAATTCGA |
| | | AGCAACGCGAAGAACCTTACCAGGTCTTGACATCCTCTGACAACCCTAGAGATAGGGCT |
| | | TTCCCTTCGGGGRCAGAGTGACAGGTGGTGCATGGTTGTCGTCAGCTCGTGTCGTGAGA |
| | | TGTTGGGTTAAGTCCCGCAACGAGCGCAACCCTTGATCTTAGTTGCCAGCATTCAGTTG |
| | | GGCACTCTAAGGTGACTGCCGGTGACAAACCGGAGGAAGGTGGGGATGACGTCAAATCA |
| | | TCATGCCCCTTATGACCTGGGCTACACACGTGCTACAATGGACAGAACAAAGGGCTGCR |
| | | AGACCGCAAGGTTTAGCCAATCCCATAAATCTGTTCTCAGTTCGGATCGCAGTCTGCAA |
| | | CTCGACTGCGTGAAGCTGGAATCGCTAGTAATCGCGGATCAGCATGCCGCGGTGAATAC |
| | | GTTCCCGGGCCTTGTACACACCGCCCGTCACACCACGAGAGTTTGCAACACCCGAAGTC |
| | | GGTGAGGTAACCTTTATGGAGCCAGCCGCCGAAGGTGGGGCAGATGATTGGGGTG |
| 2 | MS4689 | GATGAACGCTGGCGGCGTGCCTAATACATGCAAGTCGAGCGAACTRATTAGAAGCTTGC |
| | | TTCTATGACGTTAGCGGCGGACGGGTGAGTAACACGTGGGCAACCTGCCTGTAAGACTG |
| | | GGATAACTTCGGGAAACCGAAGCTAATACCGGATASGATCTTCTCCTTCATGGGAGATG |
| | | ATTGAAAGATGGTTTCGGCTATCACTTAYAGATGGGCCCGCGGTGCATTAGCTAGTTGG |
| | | TGAGGTAACGGCTCACCAAGGCAACGATGCATAGCCGACCTGAGAGGGTGATCGGCCAC |
| | | ACTGGGACTGAGACACGGCCCAGACTCCTACGGGAGGCAGCAGTAGGGAATCTTCCGCA |
| | | ATGGACGAAAGTCTGACGGAGCAACGCCGCGTGAGTGATGAAGGCTTTCGGGTCGTAAA |
| | | ACTCTGTTGTTAGGGAAGAACAAGTACDAGAGTAACTGCTYGTACCTTGACGGTACCTA |
| | | ACCAGAAAGCCACGGCTAACTACGTGCCAGCAGCCGCGGTAATACGTAGGTGGCAAGCG |
| | | TTATCCGGAATTATTGGGCGTAAAGCGCGCGCAGGCGGTTTCTTAAGTCTGATGTGAAA |
| | | GCCCACGGCTCAACCGTGGAGGGTCATTGGAAACTGGGGAACTTGAGTGCAGAAGAGAA |
| | | AAGCGGAATTCCACGTGTAGCGGTGAAATGCGTAGAGATGTGGAGGAACACCAGTGGCG |
| | | AAGGCGGCTTTTTGGTCTGTAACTGACGCTGAGGCGCGAAAGCGTGGGGAGCAAACAGG |
| | | ATTAGATACCCTGGTAGTCCACGCCGTAAACGATGAGTGCTAAGTGTTAGAGGGTTTCC |
| | | GCCCTTTAGTGCTGCAGCTAACGCATTAAGCACTCCGCCTGGGGAGTACGGTCGCAAGA |
| | | CTGAAACTCAAAGGAATTGACGGGGGCCCGCACAAGCGGTGGAGCATGTGGTTTAATTC |
| | | GAAGCAACGCGAAGAACCTTACCAGGTCTTGACATCCTCTGACAACTCTAGAGAYAGAG |
| | | CGTTCCCCTTCGGGGGACAGAGTGACAGGTGGTGCATGGTTGTCGTCAGCTCGTGTCGT |
| | | GAGATGTTGGGTTAAGTCCCGCAACGAGCGCAACCCTTGATCTTAGTTGCCAGCATTTA |
| | | GTTGGGCACTCTAAGGTGACTGCCGGTGACAAACCGGAGGAAGGTGGGGATGACGTCAA |
| | | ATCATCATGCCCCTTATGACCTGGGCTACACACGTGCTACAATGGATGGTACAAAGGGC |
| | | TGCAAGACCGYGAGGTCAAGCCAATCCCATAAAACCATTCTCAGTTCGGATTGTAGGCT |
| | | GCAACTCGCCTACATGAAGCTGGAATCGCTAGTAATCGCGGATCAGCATGCCGCGGTGA |
| | | ATACGTTCCCGGGCCTTGTACACACCGCCCGTCACACCACGAGAGTTTGTAACACCCGA |
| | | AGTCGGTGGAGTAACCGTAAGGAGCTAGCCGCCTAAGGTGGGACAGATGATTGGGGTGA |
| | | AGTCGTAACAA |
| 3 | MS4687 | CATTTCTTCGGAGAGTTTGATCCTGGCTCAGGATGAACGCTGGCGGCGTGCCTAATACA |
| | | TGCAAGTCGAGCGAACTGATTAGAAGCTTGCTTCTATGACGTTAGCGGCGGACGGGTGA |
| | | GTAACACGTGGGCAACCTGCCTGTAAGACTGGGATAACTTCGGGAAACCGAAGCTAATA |
| | | CCGGATAGGATCTTCTCCTTCATGGGAGATGATTGAAAGATGGTTTCGGCTATCACTTA |
| | | CAGATGGGCCCGCGGTGCATTAGCTAGTTGGTGAGGTAACGGCTCACCAAGGCAACGAT |
| | | GCATAGCCGACCTGAGAGGGTGATCGGCCACACTGGGACTGAGACACGGCCCAGACTCC |
| | | TACGGGAGGCAGCAGTAGGGAATCTTCCGCAATGGACGAAAGTCTGACGGAGCAACGCC |
| | | GCGTGAGTGATGAAGGCTTTCGGGTCGTAAAACTCTGTTGTTAGGGAAGAACAAGTACG |
| | | AGAGTAACTGCTCGTACCTTGACGGTACCTAACCAGAAAGCCACGGCTAACTACGTGCC |
| | | AGCAGCCGCGGTAATACGTAGGTGGCAAGCGTTATCCGGAATTATTGGGCGTAAAGCGC |
| | | GCGCAGGCGGTTTCTTAAGTCTGATGTGAAAGCCCACGGCTCAACCGTGGAGGGTCATT |
| | | GGAAACTGGGGAACTTGAGTGCAGAAGAGAAAAGCGGAATTCCACGTGTAGCGGTGAAA |
| | | TGCGTAGAGATGTGGAGGAACACCAGTGGCGAAGGCGGCTTTTTGGTCTGTAACTGACG |
| | | CTGAGGCGCGAAAGCGTGGGGAGCAAACAGGATTAGATACCCTGGTAGTCCACGCCGTA |
| | | AACGATGAGTGCTAAGTGTTAGAGGGTTTCCGCCCTTTAGTGCTGCAGCTAACGCATTA |
| | | AGCACTCCGCCTGGGGAGTACGGTCGCAAGACTGAAACTCAAAGGAATTGACGGGGGCC |
| | | CGCACAAGCGGTGGAGCATGTGGTTTAATTCGAAGCAACGCGAAGAACCTTACCAGGTC |
| | | TTGACATCCTCTGACAACTCTAGAGATAGAGCGTTCCCCTTCGGGGGACAGAGTGACAG |
| | | GTGGTGCATGGTTGTCGTCAGCTCGTGTCGTGAGATGTTGGGTTAAGTCCCGCAACGAG |
| | | CGCAACCCTTGATCTTAGTTGCCAGCATTTAGTTGGGCACTCTAAGGTGACTGCCGGTG |
| | | ACAAACCGGAGGAAGGTGGGGATGACGTCAAATCATCATGCCCCTTATGACCTGGGCTA |
| | | CACACGTGCTACAATGGATGGTACAAAGGGCTGCAAGACCGCGAGGTCAAGCCAATCCC |
| | | ATAAAACCATTCTCAGTTCGGATTGTAGGCTGCAACTCGCCTACATGAAGCTGGAATCG |
| | | CTAGTAATCGCGGATCAGCATGCCGCGGTGAATACGTTCCCGGGCCTTGTACACACCGC |
| | | CCGTCACACCACGAGAGTTTGTAACACCCGAAGTCGGTGGAGTAACCGTAAGGAGCTAG |
| | | CCGCCTAAGGTGGGACAGATGATTGGGGTGAAGTCGTAACAAGGTAGCCGTATCGGAAG |
| | | GTGCGGCTGGATCACCTCCTTTCTA |

TABLE 2
Exemplary gyrB Gene Sequences of Bacterial Isolates

| SEQ ID | | |
|---|---|---|
| NO. | Strain | Sequence |
| 4 | MS4666 | GTGGCAATGGAACAGCAACATAATAGTTATGATGAAAATCAGATACAGGTGCTTGAAGG |
| | | ATTAGAAGCTGTTAGAAAACGTCCAGGAATGTACATTGGGTCAACCAATGCAAAAGGGC |
| | | TTCACCATCTTGTATGGGAAATTGTCGACAACAGTATTGATGAGGCATTAGCTGGTTAT |
| | | TGTACAGACATTACAGTGCAAATTGAAAAAGATAACAGCATTACAGTAAAAGATAATGG |
| | | CCGCGGGATTCCTGTTGGAATTCATGAGAAAATGGGGCGTCCTGCCGTAGAGGTCATTA |
| | | TGACTGTTCTTCACGCTGGCGGTAAATTTGACGGCAGCGGTTATAAAGTATCAGGCGGT |
| | | CTGCATGGTGTAGGGGCATCTGTTGTAAATGCATTATCTACTACCTTAGACGTGACTGT |
| | | ATACCGTGACGGGAAAATCCATTATCAGCAGTTCAAACGCGGCGTTCCAGTGGGAGATT |
| | | TAGAGGTTATTGGTGAAACTGATGTAACCGGGACAACCACTCACTTTGTGCCAGATCCA |
| | | GAAATTTTCACGGAAACCATTGAATTTGATTACGATACACTTGCTAACCGTGTTCGTGA |
| | | GTTAGCCTTCTTAACAAAAGGTGTAAACATCATCATAGAAGACTTGCGTGAAGGCAAAG |
| | | AGCGGAGAAATGAATACTGCTACGAAGGCGGTATTAAGAGCTATGTAGAACATTTAAAC |
| | | CGCTCAAAAGAAGTCGTTCATGAAGAGCCTGTGTACATCGAAGGTGAGAAAGACGGAAT |
| | | CACGGTTGAAGTAGCATTACAATATAACGATTCCTATACAAGCAATATCTATTCCTTCG |
| | | CCAACAATATCAACACGTATGAAGGCGGAACACACGAAGCTGGTTTTAAAACCGGTCTA |
| | | ACGCGTGTCATCAATGACTATGCTCGTAAAAATGGCGTATTCAAAGATGGGGATGCGAA |
| | | TTTAAGTGGTGAAGATGTGCGAGAAGGCTTAACAGCCATTATCTCCATCAAACATCCAG |
| | | ACCCTCAATTCGAAGGACAAACGAAGACAAAGCTTGGGAACTCAGAAGCGAGAACCATC |
| | | ACAGACTCCCTTTTCTCTGAAGCACTTGAGAAATTCTTGCTTGAAAATCCTGATTCTGC |
| | | GAAAAAAATTGTGGAAAAAGGACTGATGGCAGCTCGTGCAAGAATGGCTGCCAAAAAAG |
| | | CTCGTGAGCTGACAAGACGTAAAAGTGCACTGGAAGTCTCCAGCTTACCTGGGAAACTG |
| | | GCGGACTGTTCTTCTAAAGATCCTTCCATCTCTGAACTCTATATTGTAGAGGGAGATTC |
| | | TGCGGGCGGATCTGCTAAACAAGGCCGTGATCGTCACTTCCAAGCGATCTTACCGTTAA |
| | | GAGGGAAGATCCTAAACGTAGAAAAAGCTCGTTTAGATAAAATTTTATCGAACAACGAG |
| | | GTTCGTTCAATGATTACAGCGTTAGGAACTGGAATCGGAGAAGACTTTACTTTAGAGAA |
| | | AGCACGCTATCACAAAATTGTGATCATGACAGATGCCGATGTGGATGGAGCACACATTC |
| | | GAACGCTGCTCTTAACATTCTTCTATCGCTACATGCGTGAAATCATTGAAAATGGTTAT |
| | | GTGTATATTGCGCAGCCGCCTTTATATAAAGTGCAGCAAGGAAAACGTGTGGAGTACGT |
| | | GTATAACGATAAACAGCTGGATGAGCTGTTAAAATCACTTCCTCAAACACCAAAGCCTG |
| | | GACTTCAGCGTTATAAAGGTCTTGGAGAAATGAATGCTGCTCAGCTTTGGGAAACAACA |
| | | ATGGACCCTGATGCGAGAACACTTCTTCAAGTCACACTTGAGGATGCGATCGATGCTGA |
| | | TGAGACATTTGAAATGCTGATGGGAGATAAAGTAGAACCGAGACGAAACTTTATCGAAG |
| | | AAAATGCGCAATACGTGAAAAACCTTGATATTTAG |
| 5 | MS4689 | ATGGAACAAAAAGAAGTACAAGCATATGAAGCTGATCAGATACAAGTATTAGAAGGATT |
| | | AGAAGCTGTTCGTAAACGTCCGGGGATGTATATTGGATCGACGAGCGCAAAGGGTTTAC |
| | | ATCATCTTGTATGGGAAATTGTAGATAATAGTATTGATGAAGCGCTGGCCGGCTATTGC |
| | | GATGAAATTAATGTTATTATCGAAAAGGATAATAGTATTACAGTCAAAGATAACGGTCG |
| | | TGGAATTCCAGTTGGTATTCAAGAAAAAATGGGCAGACCTGCTGTTGAAGTTATCTTAA |
| | | CGGTTCTTCATGCCGGAGGTAAATTTGGCGGCGGCGGCTATAAAGTATCTGGTGGATTA |
| | | CACGGTGTAGGTGCCTCAGTTGTTAACGCACTTTCTACCTCTTTGGAAGTACACGTACA |
| | | TCGTGACGGTAAAGTTCATTATCAAAAATATGAACGAGGTGTACCGGCTGCTGACTTAA |
| | | AAGTAGTTGGAGAAACAGATAAAACAGGTACTGTTATTCAATTCCATCCAGACGGTGAA |
| | | ATTTTTACAGAAACGCTTGAATACGATTTTGATACGTTAGCTAATCGTCTGCGTGAGTT |
| | | AGCTTTCTTAAACCGCGGCATTAAAATTACGATTGAAGACAAACGTGAAGAAGATAAAA |
| | | GACGTGAATACCATTACGAAGGCGGAATTAAGTCTTACGTTGAACACTTAAACCGTTCG |
| | | AAAGAAGTGATTCACGAAGAGCCGATCTATATTGAAGGTAATCGAGACAACATTTCTGT |
| | | AGAAATTGCTATTCAATATAACGATAGCTACACAAGTAATTTATATTCTTTCGCAAACA |
| | | ACATTCACACATATGAAGGTGGAACGCACGAAGCAGGATTTAAAACAGCGTTAACGCGT |
| | | GTAATTAACGACTATGCACGTAAAAACAGCGTATTTAAAGATAGTGACGCCAATTTAAC |
| | | GGGTGAAGATGTTCGTGAAGGAATTACAGCTATTATCTCTATTAAGCACCCAGATCCGC |
| | | AGTTTGAAGGACAAACAAAAACAAAGCTGGGAAATAGTGAAGCAAGAACAATTACTGAC |
| | | TCTGTGTTTGCAGAACACTTAGAAACTTACTTGCTAGAGAACCCTATTGTGGCGAAAAA |
| | | GGTAATTGAAAAAGGTTTAATGGCTGCAAGAGCAAGAATGGCAGCTAAAAAAGCTCGTG |
| | | AGCTTACAAGACGTAAAAGCGCGCTTGAAATTTCAAACTTACCGGGTAAATTAGCAGAT |
| | | TGTTCATCAAAAGATCCTTCTATTAGCGAACTCTATGTAGTAGAGGGTGACTCTGCCGG |
| | | AGGTTCAGCTAAGCAGGGAAGAAGCCGTCATTTCCAAGCTATTTTGCCTTTGCGCGGTA |
| | | AAATTATCAACGTAGAGAAAGCGCGTTTAGATAAAATTTTATCTAATAACGAAATTCGT |
| | | ACAATCATTACCGCTCTAGGAACGGGTATTGGTGACGATTTTGATATTTCGAAAGCCCG |
| | | CTACCATAAAATTGTGATTATGACAGATGCAGACGTAGATGGTGCGCATATTCGTACGC |
| | | TTCTTCTAACGTTCTTCTATCGCTATATGAGACAGATTATTGAGCACGGATATGTGTAC |
| | | ATTGCCCAGCCGCCGCTTTACAAAGTTTCACAAGGTAAAAAAGTAGAGTACGCGTACAA |
| | | CGATCGTCAATTAGAAGAGGTATTAGCTTCTTTCCCTGAAGGCGCAAAACCAAACCTTC |
| | | AGCGTTACAAAGGTTTAGGAGAGATGAATCCTGAACAATTATGGGAAACAACAATGGAT |
| | | CCAGAATTCCGTACCCTTCTTCAGGTGAACTTGCAAGATGCAATTGAAGCTGATGAGAC |
| | | ATTTGAAATTTTAATGGGCGACAAAGTAGAACCGCGCCGTAATTTCATTGAAGAAAATG |
| | | CTCAGTACGTAAAAAATCTCGATATTTAA |
| 6 | MS4687 | TTAAATATCGAGATTTTTTACGTACTGAGCATTTTCTTCAATGAAATTACGGCGCGGTT |
| | | CTACTTTGTCGCCCATTAAAATTTCAAATGTCTCATCAGCTTCAATTGCATCTTGCAAG |
| | | TTCACCTGAAGAAGGGTACGGAATTCTGGATCCATTGTTGTTTCCCATAATTGTTCAGG |
| | | ATTCATCTCTCCTAAACCTTTGTAACGCTGAAGGTTTGGTTTTGCGCCTTCAGGGAAAG |
| | | AAGCTAATACCTCTTCTAATTGACGATCGTTGTACGCGTACTCTACTTTTTTACCTTGT |
| | | GAAACTTTGTAAAGCGGCGGCTGGGCAATGTACACATATCCGTGCTCAATAATCTGTCT |
| | | CATATAGCGATAGAAGAACGTTAGAAGAAGCGTACGAATATGCGCACCATCTACGTCTG |
| | | CATCTGTCATAATCACAATTTTATGGTAGCGGGCTTTCGAAATATCAAATCGTCACCAA |
| | | TACCCGTTCCTAGAGCGGTAATGATTGTACGAATTTCGTTATTAGATAAAAATTTTATC |
| | | TAAACGCGCTTTCTCTACGTTGATAATTTTACCGCGCAAAGGCAAAATAGCTTGGAAAT |
| | | GACGGCTTCTTCCCTGCTTAGCTGAACCTCCGGCAGAGTCACCCTCTACTACATAGAGT |
| | | TCGCTAATAGAAGGATCTTTTGATGAACAATCTGCTAATTTACCCGGTAAGTTTGAAAT |
| | | TTCAAGCGCGCTTTTACGTCTTGTAAGCTCACGAGCTTTTTTTAGCTGCCATTCTTGCT |
| | | CTTGCAGCCATTAAACCTTTTTCAATTACCTTTTCGCCACAATAGGGTTCTCTAGCAAG |
| | | TAAGTTTCTAAGTGTTCTGCAAACACAGAGTCAGTAATTGTTCTTGCTTCACTATTTCC |
| | | CAGCTTTGTTTTTGTTTGTCCTTCAAACTGCGGATCTGGGTGCTTAATAGAGATAATAG |
| | | CTGTAATTCCTTCACGAACATCTTCACCCGTTAAATTGGCGTCACTATCTTTAAATACG |
| | | CTGTTTTTACGTGCATAGTCGTTAATTACACGCGTTAACGCTGTTTTAAATCCTGCTTC |
| | | GTGCGTTCCACCTTCATAATGTGTGAATGTTGTTTGCGAAAGAATATAAATTACTTGTG |
| | | TAGCTATCGTTATATTGAATAGCAATTTCTACAGAAATGTTGTCTCGATTACCTTCAAT |
| | | ATAGATCGGCTCTTCGTGAATCACTTCTTTTCGAACGGTTTAAGTGTTCAACGTAAGAC |
| | | TTAATTCCGCCTTCGTAATGGTATTCACGTCTTTTATCTTCTTCACGTTTGTCTTCAAT |
| | | CGTAATTTTAATGCCGCGGTTTAAGAAAGCTAACTCACGCAGACGATTAGCTAACGTAT |
| | | CAAAATCGTATTCAAGCGTTTCTGTAAAAATTTCACCGTCTGGATGGAATTGAATAACA |
| | | GTACCTGTTTTATCTGTTTCTCCAACTACTTTTAAGTCAGCAGCCGGTACACCTCGTTC |
| | | ATATTTTTGATAATGAACTTTACCGTCACGATGTACGTGTACTTCCAAAGAGGTAGAAA |
| | | GTGCGTTAACAACTGAGGCACCTACACCGTGTAATCCACCAGATACTTTATAGCCGCCG |
| | | CCGCCAAATTTACCTCCGGCATGAAGAACCGTTAAGATAACTTCAACAGCAGGTCTGCC |
| | | CATTTTTTCTTGAATACCAACTGGAATTCCACGACCGTTATCTTTGACTGTAATACTAT |
| | | TATCCTTTTCGATAATAACATTAATTTCATCGCAATAGCCGGCCAGCGCTTCATCAATA |
| | | CTATTATCTACAATTTCCCATACAAGATGATGTAAACCCTTTGCGCTCGTCGATCCAAT |
| | | ATACATCCCCGGACGTTTACGAACAGCTTCTAATCCTTCTAATACTTGTATCTGATCAG |
| | | CTTCATATGCTTGTACTTCTTTTTGTTTCCATCGTCAA |

TABLE 3
Exemplary rpoB Gene Sequences of Bacterial Isolates

| SEQ ID | | |
|---|---|---|
| NO. | Strain | Sequence |
| 7 | MS4666 | ATGTTTCAAGATATATCCCCAATTGAGGATTTTACTGGTAACCTTTCTCTTGAATTCAT |
| | | TGATTACAGCCTAGGGGATCCTAAGTATCCTGTAGCAGAATCAAAAGAACGTGATGTAA |
| | | CTTACTCTGCTCCACTAAGAGTAAAAGTTCGTTTAATTAACAAAGAAACTGGAGAAGTA |
| | | AAAGACCAAGATGTGTTCATGGGTGATTTCCCAATCATGACAGACACAGGTACTTTTAT |
| | | CATTAATGGTGCGGAACGTGTAATCGTTTCTCAGTTAGTGCGTTCTCCAAGTGTATATT |
| | | TCAGTGGTAAAGTAGACAAAAACGGTAAAAAAGGTTTTACTGCGACTGTCATTCCAAAC |
| | | CGTGGCGCATGGTTAGAATACGAAACTGATGCGAAGGATGTAGTCTATGTACGCATCGA |
| | | TCGCACACGTAAGTTGCCGGTTACGGTTCTTTTGCGTGCTCTCGGCTTCAGCTCTGATC |
| | | AAGAGATTCTTGACCTCATTGGCGAGAATGAATACTTACGCAACACACTGGAAAAAGAC |
| | | AATACAGAGAATGCGGATAAAGCACTTCTCGAAATCTACGAGCGCCTCCGTCCTGGAGA |
| | | ACCGCCAACTGTTGAAAATGCGAAAAGCTTGCTAGACTCCCGCTTCTTCGATCCGAAGA |
| | | GATATGACCTAGCAAATGTTGGACGCTACAAGATTAATAAGAAGCTTCATATTAAAAAT |
| | | AGACTGTTCAATCAAAAATTGGCTGAAACGCTAGTTGATCCTGAAACAGGTGAAATTCT |
| | | AGCAGAAAAAGGTCAAATTTTAGACAGAAGAGTTCTTGATAAAGTTCTTCCATACTTAG |
| | | AAAACGGCATCGGATTTAGAAAGCTTTATCCTAATGGTGGCGTAGTAGAAGATGAAGTA |
| | | GAACTTCAATCTATTAAGATTTATGCACCGACTGATCAAGAAGGCGAGCAAGTGATCAA |
| | | CGTGATTGGAAATGCATATGTAGAGGAAGCTGTGAAAAACATCACGCCTTCTGACATCA |
| | | TTGCGTCAATCAGTTACTTCTTCAACCTTCTTCATGGTGTAGGTGACACAGATGATATC |
| | | GACCACCTTGGTAACCGTCGTCTGCGTTCTGTAGGTGAACTTCTGCAAAACCAATTCCG |
| | | TATTGGTTTAAGCAGAATGGAGCGTGTTGTCCGTGAAAGAATGTCAATTCAAGACACAA |
| | | ACACGATCACACCTCAGCAGCTGATCAACATTCGTCCTGTGATCGCTTCTATCAAAGAG |
| | | TTCTTTGGTAGTTCTCAGCTTTCTCAGTTCATGGATCAAACAAACCCGCTTGCTGAATT |
| | | GACGCACAAACGTCGTCTGTCAGCGCTTGGACCGGGTGGTTTGACACGTGAGCGTGCAG |
| | | GAATGGAAGTTCGTGACGTTCACTACTCTCACTATGGACGTATGTGTCCGATTGAAACA |
| | | CCAGAGGGTCCAAACATTGGTTTGATCAACTCTCTATCTTCATTTGCAAAAGTGAATCG |
| | | CTTCGGATTTATCGAAACACCTTACCGCCGGGTTGATCCTGAAACTGGTAAAGTGACAC |
| | | CGAGAATCGATTACTTAACTGCTGATGAAGAGGATAACTACGTAGTAGCACAAGCGAAC |
| | | GCTCAGTTAGCTGATGATGGTTCGTTTATCGATGATAATATCATTGCTCGTTTCAGAGG |
| | | GGAAAACACCGTTGTTCCTCGAAACCGCGTTGACTACATGGACGTTTCGCCAAAGCAGG |
| | | TTGTTTCTGCAGCGACAGCATGTATCCCATTCTTAGAAAACGATGACTCTAACCGTGCT |
| | | CTAATGGGAGCGAACATGCAGCGTCAGGCTGTGCCTTTGATGCAGCCAGAATCACCGAT |
| | | TGTTGGTACAGGTATGGAGTACGTATCAGGTAAAGACTCTGGTGCTGCTGTCATCTGCC |
| | | GATACCCAGGTGTTGTAGAACGTGTAGAAGCAAAAAATATTTGGGTTCGCCGTTATGAA |
| | | GATGTTGATGGACAACAAGTCAAAGGAAACCTAGACAAATACAGCTTGCTGAAATTTGT |
| | | CCGCTCTAACCAAGGGACTTGCTACAACCAGCGTCCTATCGTAAGTGTTGGAGATGAAG |
| | | TCGTTAAAGGAGAAATCCTTGCAGACGGACCTTCTATGGAAAAAGGTGAATTGGCTCTA |
| | | GGACGTAACGTTATGGTCGGCTTCATGACTTGGGACGGTTACAACTACGAGGATGCGAT |
| | | CATCATGAGTGAGCGCCTTGTAAAAGATGACGTCTACACGTCTATTCATATTGAAGAAT |
| | | ATGAATCAGAAGCTCGTGATACAAAGCTTGGACCAGAAGAAATCACTCGTGATATCCCA |
| | | AACGTTGGGGAAGATGCTTTACGCAACCTTGACGAGCGCGGAATCATCCGTATCGGTGC |
| | | AGAAGTAAAAGACGGAGACCTTCTTGTAGGAAAAGTAACGCCTAAAGGTGTAACTGAAC |
| | | TAACAGCTGAAGAACGTCTATTACATGCAATCTTCGGTGAAAAGGCTCGTGAAGTTCGT |
| | | GATACGTCACTACGTGTTCCACACGGCGGCGGCGGAATTATCCACGATGTAAAAGTCTT |
| | | CAACCGTGAAGATGGAGATGAATTACCTCCAGGTGTAAACCAGTTAGTCCGCGTATACA |
| | | TCGTTCAGAAGCGTAAGATTTCTGAAGGTGACAAAATGGCCGGACGACATGGTAACAAA |
| | | GGGGTTATCTCTAAAATCCTTCCAGAAGAAGATATGCCTTATCTTCCAGACGGAACACC |
| | | GATCGATATCATGTTGAACCCTCTAGGGGTACCATCTCGTATGAACATCGGTCAGGTAC |
| | | TTGAGCTTCATATGGGTATGGCTGCACGTTACCTCGGCATCCACATTGCGTCACCAGTA |
| | | TTTGATGGTGCGCGCGAGGAAGATGTTTGGGAAACGCTTGAAGAAGCAGGTATGTCCCG |
| | | TGATGCGAAAACAGTCCTTTATGACGGTCGAACTGGTGAACCATTCGACAACCGTGTAT |
| | | CTGTCGGAATCATGTACATGATCAAACTGGCTCACATGGTTGACGATAAACTTCACGCT |
| | | CGTTCAACTGGACCATACTCACTTGTTACGCAGCAGCCACTTGGCGGTAAAGCACAGTT |
| | | TGGTGGTCAGCGTTTCGGAGAGATGGAAGTATGGGCACTTGAAGCTTACGGTGCAGCAT |
| | | ATACACTTCAAGAGATCTTAACAGTTAAATCGGATGACGTTGTGGGTCGTGTGAAAACA |
| | | TACGAAGCCATCGTCAAAGGGGATAACGTACCTGAACCAGGTGTCCCTGAATCATTCAA |
| | | AGTGTTAATCAAAGAACTTCAAAGTTTAGGTATGGATGTGAAAATCCTATCTGGCGATG |
| | | AAGAAGAGATAGAAATGAGAGATTTAGAAGACGATGAGGAAACGAAGAAAGCAGACGGA |
| | | TTAGCGTTATCTAATGACGAAGATGCTGCAGACCTCGCACCTGTCGATCTTGAACGTGA |
| | | CGCAGTCACAAAAGAATAG |
| 8 | MS4689 | TTGACAGGTCAACTAGTTCAGTATGGACGCCACCGCCAACGCAGAAGTTATGCTCGTAT |
| | | TAGTGAAGTTTTAGAGTTACCGAACTTAATCGAGATTCAAACGGCTTCATACCAATGGT |
| | | TTTTAGATGAAGGTTTACGAGAAATGTTCCAAGATATTTCTCCAATTGATGATTTTACA |
| | | GGTAACTTATCACTAGAATTTATTGATTACAGCTTAGGTGAGCCAAAGTATTCAGTAGG |
| | | AGAGTCTAAAGAACGTGATGTAACTTATGCAGCACCTCTTCGTGTTAAGGTGCGGTTAA |
| | | TTAATAAAGAAACAGGTGAAGTAAAAGACCAAGATGTGTTCATGGGAGATTTCCCATTG |
| | | ATGACCGAAACAGGTACCTTTGTAATTAATGGTGCTGAGCGTGTTATCGTATCACAGTT |
| | | GGTTCGTTCACCAAGTGTTTACTATAGTGGAAAGCTTGATAAAAACGGTAAAAAAGGAT |
| | | ATACAGCAACTGTTATCCCTAACCGTGGTGCATGGCTAGAATATGAAACTGATGCAAAA |
| | | GATGTAGTATACGTGCGTATTGATCGTACTCGTAAACTGCCAGTAACAGTGCTGTTACG |
| | | CGCGCTAGGTTTCGGTTCTGACCAAGAGATTATTGATTTAATCGGTGATAATGAATACA |
| | | TCCGTAATACGCTTGAAAAAGATAATACGGAAACAACGGAAAAAGCGCTATTAGAAATC |
| | | TATGAGCGTTTACGTCCGGGTGAACCACCGACAGTTGAGAATGCGAAGTCTCTATTAGT |
| | | ATCTCGCTTCTTTGATCCAAAACGATATGACTTAGCGAACGTAGGTCGCTATAAAATTA |
| | | ATAAAAAGCTTCATATCAAACATCGCTTATTTAATCAAAAGCTAGCTGAAACATTAGTT |
| | | GATCCTGAAACAGGTGAAATTATTGCAGAAAAAGGCGCAATGATTGATCGCCGTCTGCT |
| | | AGATCGCTTGATTCCGATGCTTGAAGGTGGAGTAAACTTCAAAACTTATAGTCCGGTTG |
| | | GTGGAGTAGTAGAAGACGATGTTACATTACAATCTATTAAGATTTATGCGCCAAATGAT |
| | | CCAGAAGGTGAAAAAGTCATCACTGTATCAGGCAACGCTTATGTAACAGAAGAAGTTAA |
| | | AAATATCACACCTGCTGATATTTTAGCATCAATCAGTTACTTCTTTAACTTGCTTCATC |
| | | AAGTAGGAGACACAGATGATATCGACCACTTAGGTAACCGTCGTCTGCGTTCTGTAGGT |
| | | GAATTGTTACAAAACCAATTCCGTATCGGTTTATCTCGTATGGAACGTGTTGTTCGTGA |
| | | AAGAATGTCAATTCAAGACACGAATACAATCACACCTCAACAATTAATTAATATTCGTC |
| | | CAGTTATTGCGGCGATTAAAGAGTTCTTTGGAAGTTCTCAATTATCACAGTTCATGGAT |
| | | CAAACGAATCCATTAGGCGAATTGACGCACAAACGTCGTCTTTCAGCTCTAGGACCTGG |
| | | TGGTTTAACGCGTGAACGCGCTGGTTTCGAAGTGCGTGACGTTCACTACTCCCACTATG |
| | | GCCGTATGTGTCCGATTGAAACGCCAGAGGGTCCGAATATCGGGTTAATCAACTCACTT |
| | | TCTTCATATGCAAAAGTAAACAAATTCGGTTTCATCGAAACACCTTACCGTCGTATCGA |
| | | TCCTGAAACAGGTAAAGTGACAGAGCGAATTGACTACTTAACAGCTGATGAAGAAGATA |
| | | ACTATGTTGTAGCCCAAGCGAACGCTCGTCTAGGTGATGATGGTTCATTCTTAGATGAA |
| | | AATGTCGTTGCACGTTTCCGTGGAGAAAACACGGTTATCCGTCGCGATCGTTTAGACTA |
| | | TATGGATGTATCACCAAAACAAGTTGTATCTGCCGCTACAGCATGTATCCCATTCTTAG |
| | | AGAACGATGACTCTAACCGTGCATTAATGGGTGCGAACATGCAACGTCAAGCAGTACCT |
| | | TTATTAAATCCTGAAGCACCAATCGTAGGTACAGGTATGGAATATGTATCTGGTAAAGA |
| | | CTCTGGTGCAGCCGTGATTTGTAAATACCCTGGCGTTGTAGAGCGCGTAGAAGCAAAAC |
| | | AAATTTTTGTTCGCCGCTATGAAGAAGTAGACGGACAAAAAGTTAAAGGTAACTTAGAT |
| | | CAATACAAATTATTAAAATTCGTTCGTTCTAACCAAGGTACTTGTTACAACCAGCGTCC |
| | | AATTGTTTCAGTTGGCGACGAAGTAGTAAAAGGTGAGATCTTAGCCGACGGTCCTTCAA |
| | | TGGAAAAAGGTGAGCTTGCTTTAGGACGAAACGTAATGGTTGGTTTCATGACATGGGAT |
| | | GGTTACAACTATGAGGATGCCATCATCATGAGTGAACGCCTTGTGAAAGACGATGTATA |
| | | TACGTCTGTTCATATTGAAGAATATGAATCTGAGTCTCGTGATACGAAACTTGGACCTG |
| | | AAGAAATTACGCGTGACATTCCAAACGTAGGTGAAGATGCGCTTCGCAACTTAGATGAG |
| | | CGTGGAATCATCCGCATTGGTGCAGAAGTAAAAGATGGAGATCTTTTAGTTGGTAAAGT |
| | | AACGCCAAAAGGTGTAACAGAACTAACAGCTGAAGAACGTCTTCTACACGCTATTTTCG |
| | | GTGAAAAAGCTCGTGAAGTTCGTGATACTTCTCTTCGTGTACCGCACGGCGGCGGTGGA |
| | | ATTATCCTTGATGTTAAAGTCTTCAACCGTGAAGATGGCGACGAATTACCACCAGGTGT |
| | | AAACCAATTAGTCCGTGTATACATTGTTCAGAAGCGTAAAATTTCTGAAGGTGACAAAA |
| | | TGGCCGGTCGTCACGGTAACAAAGGTGTAATTTCACGTATTTTACCGGAAGAAGATATG |
| | | CCTTACCTACCAGACGGTACGCCAATTGACATCATGTTAAACCCATTAGGGGTACCATC |
| | | TCGTATGAACATCGGTCAGGTGCTAGAGCTTCATTTAGGTATGGCTGCTCGTAAGCTTG |
| | | GCATTCACGTTGCGTCTCCAGTATTTGATGGTGCGCGTGAGGAAGATGTTTGGGCAACA |
| | | ATCGAAGAAGCTGGCATGTCTCGTGATGCTAAAACAGTTCTATATGATGGTCGAACAGG |
| | | TGAACCATTCGATAACCGTGTATCAGTAGGAATCATGTACATGATCAAACTTGCTCACA |
| | | TGGTAGACGATAAACTTCACGCTCGTTCTACTGGACCATACTCACTTGTTACACAACAA |
| | | CCACTTGGTGGTAAAGCGCAGTTCGGTGGACAGCGTTTTGGTGAGATGGAAGTATGGGC |
| | | ACTTGAAGCATACGGTGCTGCTTATACATTACAAGAGATCTTAACAGTGAAATCAGACG |
| | | ACGTAGTAGGTCGTGTGAAAACATACGAAGCAATTGTTAAAGGTGAAAACATTCCAGAA |
| | | CCTGGCATACCTGAATCGTTCAAAGTATTAATCAAAGAACTACAAAGTTTAGGTATGGA |
| | | TGTGAAGATGCTTTCTGCTGACGAGCAAGAAATTGATATCATGGACTCAGAAGAGGACC |
| | | ATGAGCAACCAACAGAATCAATTATTGCAGATAACGAAGAAAGCCTTTCTGAAGGACAA |
| | | AAAGATCCTGTCACAAAAGAGTAA |
| 9 | MS4687 | TTACTCTTTTGTGACAGGATCTTTTTGTCCTTCAGAAAGGCTTTCTTCGTTATCTGCAA |
| | | TAATTGATTCTGTTGGTTGCTCATGGTCCTCTTCTGAGTCCATGATATCAATTTCTTGC |
| | | TCGTCAGCAGAAAGCATCTTCACATCCATACCTAAACTTTGTAGTTCTTTGATTAATAC |
| | | TTTGAACGATTCAGGTATGCCAGGTTCTGGAATGTTTTCACCTTTAACAATTGCTTCGT |
| | | ATGTTTTCACACGACCTACTACGTCGTCTGATTTCACTGTTAAGATCTCTTGTAATGTA |
| | | TAAGCAGCACCGTATGCTTCAAGTGCCCATACTTCCATCTCACCAAAACGCTGTCCACC |
| | | GAACTGCGCTTTACCACCAAGTGGTTGTTGTGTAACAAGTGAGTATGGTCCAGTAGAAC |
| | | GAGCGTGAAGTTTATCGTCTACCATGTGAGCAAGTTTGATCATGTACATGATTCCTACT |
| | | GATACACGGTTATCGAATGGTTCACCTGTTCGACCATCATATAGAACTGTTTTAGCATC |
| | | ACGAGACATGCCAGCTTCTTCGATTGTTGCCCAAACATCTTCCTCACGCGCACCATCAA |
| | | ATACTGGAGACGCAACGTGAATGCCAAGCTTACGAGCAGCCATACCTAAATGAAGCTCT |
| | | AGCACCTGACCGATGTTCATACGAGATGGTACCCCTAATGGGTTTAACATGATGTCAAT |
| | | TGGCGTACCGTCTGGTAGGTAAGGCATATCTTCTTCCGGTAAAATACGTGAAATTACCC |
| | | TTTGTTACCGTGACGACCGGCCATTTTGTCACCTTCAGAAATTTTACGCTTCTGAACAA |
| | | TGTATACACGGACTAATTGGTTTACACCTGGTGGTAATTCGTCGCCATCTTCACGGTTG |
| | | AAGACTTTAACATCAAGGATAATTCCACCGCCGCCGTGCGGTACACGAAGAGAAGTATC |
| | | ACGAACTTCACGCGCTTTTTCACCGAAAATAGCGTGTAGAAGACGTTCTTCAGCTGTTA |
| | | GTTCTGTTACACCTTTTGGCGTTACTTTACCAACTAAAAGATCTCCATCTTTTACTTCT |
| | | GCACCAATGCGGATGATTCCACGCTCATCTAAGTTGCGAAGCGCATCTTCACCTACGTT |
| | | TGGAATGTCACGCGTAATTTCTTCAGGTCCAAGTTTCGTATCACGAGACTCAGATTCAT |
| | | ATTCTTCAATATGAACAGACGTATATACATCGTCTTTCACAAGGCGTTCACTCATGATG |
| | | ATGGCATCCTCATAGTTGTAACCATCCCATGTCATGAAACCAACCATTACGTTTCGTCC |
| | | TAAAGCAAGCTCACCTTTTTCCATTGAAGGACCGTCGGCTAAGATCTCACCTTTTACTA |
| | | CTTCGTCGCCAACTGAAACAATTGGACGCTGGTTGTAACAAGTACCTTGGTTAGAACGA |
| | | ACGAATTTTAATAATTTGTATTGATCTAAGTTACCTTTAACTTTTTGTCCGTCTACTTC |
| | | TTCATAGCGGCGAACAAAAATTTGTTTTGCTTCTACGCGCTCTACAACGCCAGGGTATT |
| | | TACAAATCACGGCTGCACCAGAGTCTTTACCAGATACATATTCCATACCTGTACCTACG |
| | | ATTGGTGCTTCAGGATTTAATAAAGGTACTGCTTGACGTTGCATGTTCGCACCCATTAA |
| | | TGCACGGTTAGAGTCATCGTTCTCTAAGAATGGGATACATGCTGTAGCGGCAGATACAA |
| | | CTTGTTTTGGTGATACATCCATATAGTCTAAACGATCGCGACGGATAACCGTGTTTTCT |
| | | CCACGGAAACGTGCAACGACATTTTCATCTAAGAATGAACCATCATCACCTAGACGAGC |
| | | GTTCGCTTGGGCTACAACATAGTTATCTTCTTCATCAGCTGTTAAGTAGTCAATTCGCT |
| | | CTGTCACTTTACCTGTTTCAGGATCGATACGACGGTAAGGTGTTTCGATGAAACCGAAT |
| | | TTGTTTACTTTTGCATAAGAAGAAAGTGAGTTGATTAACCCGATATTCGGACCCTCTGG |
| | | CGTTTCAATCGGACACATACGGCCATAGTGGGAGTAGTGAACGTCACGCACTTCGAAAC |
| | | CAGCGCGTTCACGCGTTAAACCACCAGGTCCTAGAGCTGAAAGACGACGTTTGTGCGTC |
| | | AATTCGCCTAATGGATTCGTTTGATCCATGAACTGTGATAATTGAGAACTTCCAAAGAA |
| | | CTCTTTAATCGCCGCAATAACTGGACGAATATTAATTAATTGTTGAGGTGTGATTGTAT |
| | | TCGTGTATGAATTGGACATTCTTTCACGAACAACACGTTCCATACGAGATAAACCGATA |
| | | CGGAATTGGTTTTGTAACAATTCACCTACAGAACGCAGACGACGGTTACCTAAGTGGTC |
| | | GATATCATCTGTGTCTCCTACTTGATGAAGCAAGTTAAAGAAGTAACTGATTGATGCTA |
| | | AAATATCAGCAGGTGTGATATTTTTAACTTCTTCTGTTACATAAGCGTTGCCTGATACA |
| | | GTGATGACTTTTTCACCTTCTGGATCATTTGGCGCATAAATCTTAATAGATTGTAATGT |
| | | AACATCGTCTTCTACTACTCCACCAACCGGACTATAAGTTTGAAGTTTACTCCACCTTC |
| | | AAGCATCGGAATCAAGCGATCTAGCAGACGGCGATCAATCATTGCGCCTTTTTCTGCAA |
| | | TAATTTCACCTGTTTCAGGATCAACTAATGTTTCAGCTAGCTTTTGATTAAATAAGCGA |
| | | TGTTTGATATGAAGCTTTTTATTAATTTTATAGCGACCTACGTTCGCTAAGTCATATCG |
| | | TTTTGGATCAAAGAAGCGAGATACTAATAGAGACTTCGCATTCTCAACTGTCGGTGGTT |
| | | CACCCGGACGTAAACGCTCATAGATTTCTAATAGCGCTTTTTCCGTTGTTTCCGTATTA |
| | | TCTTTTTCAAGCGTATTACGGATGTATTCATTATCACCGATTAAATCGATAATCTCTTG |
| | | GTCAGAACCGAAACCTAGCGCGCGTAACAGCACTGTTACTGGCAGTTTACGAGTACGAT |
| | | CAATACGCACGTATACTACATCTTTTGCATCAGTTTCATATTCTAGCCATGCACCACGG |
| | | TTAGGGATAACATTGCTGTATATCCTTTTTTACCGTTTTTATCAAGCTTTCCACTATAG |
| | | TAAACACTTGGTGAACGAACCAACTGTGATACGATAACACGCTCAGCACCATTAATTAC |
| | | AAAGGTACCTGTTTCGGTCATCAATGGGAAATCTCCCATGAACACATCTTGGTCTTTTA |
| | | CTTCACCTGTTTCTTTATTAATTAACCGCACCTTAACACGAAGAGGTGCTGCATAAGTT |
| | | ACATCACGTTCTTTAGACTCTCCTACTGAATACTTTGGCTCACCTAAGCTGTAATCAAT |
| | | AAATTCTAGTGATAAGTTACCTGTAAATCATCAATTGGAGAAATATCTTGGAACATTTC |
| | | TCGTAAACCTTCATCTAAAAACCATTGGTATGAAGCGTTTGAATCTCGATTAAGTTCGG |
| | | TAACTCTAAAACTTCACTAATACGAGCATAACTTCTGCGTTGGCGGTGGCGTCCATACT |
| | | GAACTAGTTGACCTGTCAA |

Incubation of a microbial consortium in the bioreactor system under conditions of a selective pressure source (e.g., zinc source) may enrich a microbial community with plant growth promotion properties (e.g., zinc solubilizing microbes). Incubation of the inoculum of the microbe and/or the zinc-solubilizing microbes of the microbial consortium may generate metabolites with desired plant growth promotion properties (e.g., zinc-solubilizing metabolites. The inoculum of the microbe, zinc-solubilizing microbes of the microbial consortium, zinc-solubilizing metabolites, or any combination thereof may be zinc-solubilizers in the working fluid of the system and/or in the output product (e.g., base product) of the bioreactor system. A concentration of zinc solubilizers may increase throughout a retention time of a bioreactor system described herein. The concentration of zinc solubilizers may increase from a first container to a second container of the bioreactor system. The concentration of zinc solubilizers may increase from a first container to a third container of the bioreactor system. The concentration of zinc solubilizers may increase from a first container to a fourth container of the bioreactor system. The concentration of zinc solubilizers may increase from a first container to a fifth container of the bioreactor system. The concentration of zinc solubilizers may increase from a first container to a sixth container of the bioreactor system. In some embodiments, an output product (e.g., base product) may have a higher concentration of zinc solubilizers than a concentration of zinc solubilizers in a first container of a bioreactor system.

In some embodiments, a concentration of zinc-solubilizers may increase from a first container to a second container, a third container, a fourth container, a fifth container, and/or a sixth container of a bioreactor system described herein. In some embodiments, a concentration of zinc-solubilizers in a second container, a third container, a fourth container, a fifth container, and/or a sixth container may be at least about 50×, at least about 100×, at least about 200×, at least about 250×, at least about 300×, at least about 400×, at least about 500×, at least about 600×, at least about 700×, at least about an 800×, at least about 900×, at least about 1000×, at least about 1250×, at least about 1500×, at least about 2000×, or greater than about 2000× increased from a concentration of zinc-solubilizers in a first container of a bioreactor system described herein. In some embodiments, a concentration of zinc-solubilizers in a second container, a third container, a fourth container, a fifth container, and/or a sixth container may be at most about 2000×, at most about 1500×, at most about 1250×, at most about 1000×, at most about 900×, at most about an 800×, at most about 700×, at most about 600×, at most about 500×, at most about 400×, at most about 300×, at most about 250×, at most about 200×, at most about 100×, at most about 50×, or less than about 50× increased from a concentration of zinc-solubilizers in a first container of a bioreactor system described herein.

In some embodiments, a concentration of zinc-solubilizers in a second container, a third container, a fourth container, a fifth container, and/or a sixth container may be from about 25× to about 2,500× increased from a concentration of zinc-solubilizers in a first container of a bioreactor system described herein. In some embodiments, a concentration of zinc-solubilizers in a second container, a third container, a fourth container, a fifth container, and/or a sixth container may be from about 25× to about 50×, about 25× to about 100×, about 25× to about 150×, about 25× to about 200×, about 25× to about 250×, about 25× to about 500×, about 25× to about 750×, about 25× to about 1,000×, about 25× to about 1,500×, about 25× to about 2,000×, about 25× to about 2,500×, about 50× to about 100×, about 50× to about 150×, about 50× to about 200×, about 50× to about 250×, about 50× to about 500×, about 50× to about 750×, about 50× to about 1,000×, about 50× to about 1,500×, about 50× to about 2,000×, about 50× to about 2,500×, about 100× to about 150×, about 100× to about 200×, about 100× to about 250×, about 100× to about 500×, about 100× to about 750×, about 100× to about 1,000×, about 100× to about 1,500×, about 100× to about 2,000×, about 100× to about 2,500×, about 150× to about 200×, about 150× to about 250×, about 150× to about 500×, about 150× to about 750×, about 150× to about 1,000×, about 150× to about 1,500×, about 150× to about 2,000×, about 150× to about 2,500×, about 200× to about 250×, about 200× to about 500×, about 200× to about 750×, about 200× to about 1,000×, about 200× to about 1,500×, about 200× to about 2,000×, about 200× to about 2,500×, about 250× to about 500×, about 250× to about 750×, about 250× to about 1,000×, about 250× to about 1,500×, about 250× to about 2,000×, about 250× to about 2,500×, about 500× to about 750×, about 500× to about 1,000×, about 500× to about 1,500×, about 500× to about 2,000×, about 500× to about 2,500×, about 750× to about 1,000×, about 750× to about 1,500×, about 750× to about 2,000×, about 750× to about 2,500×, about 1,000× to about 1,500×, about 1,000× to about 2,000×, about 1,000× to about 2,500×, about 1,500× to about 2,000×, about 1,500× to about 2,500×, or about 2,000× to about 2,500× increased from a concentration of zinc-solubilizers in a first container of a bioreactor system described herein.

In some embodiments, the absolute population of the microbial inoculum of the bioreactor system may not decrease by more than about 0.000001%, 0.00001%, 0.0001%, 0.001%, 0.01%, 0.1%, 0.5%, 1%, 2%, 3%, 4%, 5%, 6%, 7%, 8%, 9%, 10%, 15%, 20%, or 25% after incubation. In some embodiments, the absolute population of the microbial inoculum of the bioreactor system may not decrease by more than about 0.000001%, 0.00001%, 0.0001%, 0.001%, 0.01%, 0.1%, 0.5%, 1%, 2%, 3%, 4%, 5%, 6%, 7%, 8%, 9%, 10%, 15%, 20%, or 25% after incubation for a retention period of a bioreactor system (e.g., 1 day, 2 days, 3 days, 4 days, 5 days, 6 days, 1 week, 2 weeks, 3 weeks, 4 weeks, 2 months, 3 months, 4 months, 5 months, 6 months, 1 year, 2 years, 3 years, 4 years, or 5 years). In some embodiments, the absolute population of the microbial inoculum of the bioreactor system may not decrease by more than about 0.000001%, 0.00001%, 0.0001%, 0.001%, 0.01%, 0.1%, 0.5%, 1%, 2%, 3%, 4%, 5%, 6%, 7%, 8%, 9%, 10%, 15%, 20%, or 25% after incubation without inoculation of additional microbe.

In some embodiments, a proportion of a concentration of zinc solubilizers relative to a total bacterial population count may increase across containers of a fluidly connected bioreactor system or may stay the same over a given period of time. In some embodiments, a proportion of a concentration zinc solubilizers relative to a total bacterial population count may increase from a first container to a second container of a bioreactor system described herein. In some embodiments, a proportion of a concentration zinc solubilizers relative to a total bacterial population count may increase from a first container to a third container of a bioreactor system described herein. In some embodiments, a proportion of a concentration zinc solubilizers relative to a total bacterial population count may increase from a first container to a fourth container of a bioreactor system described herein. In some embodiments, a proportion of a concentration zinc solubilizers relative to a total bacterial population count may increase from a first container to a fifth container of a bioreactor system described herein. In some embodiments, a proportion of a concentration zinc solubilizers relative to a total bacterial population count may increase from a first container to a sixth container of a bioreactor system described herein.

Zinc solubilization can result in the production of zinc ions (Zn2+) as an insoluble source of zinc is solubilized by microbes described herein. A measure of zinc solubilization and capacity of microbes to solubilize zinc can be an amount of zinc ions (Zn2+) made soluble. In some embodiments, the zinc solubilization capacity (e.g., as Zn2+mg/L made soluble) of a working solution may increase in a bioreactor system provided herein. In some embodiments, the zinc solubilization capacity (e.g., as Zn2+mg/L made soluble) of a working solution may be at least about 50× higher, at least about 75× higher, at least about 100× higher, at least about 150× higher, at least about 200× higher, at least about 250× higher, at least about 300× higher, at least about 350× higher, at least about 400× higher, at least about 450× higher, at least about 500× higher, at least about 600× higher, at least about 700× higher, at least about 800× higher, at least about 900× higher, at least about 1000× higher, or greater than about 1,000× higher in a second container, third container, or fourth container, compared to that in a first container of the bioreactor system. In some embodiments, the zinc solubilization capacity (e.g., as Zn2+mg/L made soluble) of a working solution may be at most about 1,000× higher, at most about 900× higher, at most about 800× higher, at most about 700× higher, at most about 600× higher, at most about 500× higher, at most about 450× higher, at most about 400× higher, at most about 350× higher, at most about 300× higher, at most about 250× higher, at most about 200× higher, at most about 150× higher, at most about 100× higher, at most about 75× higher, at most about 50× higher, or less than about 50× higher in a second container, third container, or fourth container, compared to that in a first container of the bioreactor system.

In some embodiments, a working solution may be obtained from a reactor of a bioreactor system and tested for zinc solubilizing capacity (e.g., quantified as Zn2+mg/L made soluble). In some embodiments, a working solution of a bioreactor system described herein may have a measure of Zn2+ made soluble of at least about 50 mg/L, at least about 100 mg/L, at least about 150 mg/L, at least about 200 mg/L, at least about 250 mg/L, at least about 300 mg/L, at least about 350 mg/L, at least about 400 mg/L, at least about 450 mg/L, at least about 500 mg/L, at least about 600 mg/L, at least about 700 mg/L, at least about 800 mg/L, or greater than about 800 mg/L. In some embodiments, a working solution of a bioreactor system described herein may have a measure of Zn2+ made soluble of at most about 800 mg/L, at most about 700 mg/L, at most about 600 mg/L, at most about 500 mg/L, at most about 450 mg/L, at most about 400 mg/L, at most about 350 mg/L, at most about 300 mg/L, at most about 250 mg/L, at most about 200 mg/L, at most about 150 mg/L, at most about 100 mg/L, at most about 50 mg/L, or less than about 50 mg/L. In some embodiments, a working solution of a bioreactor system described herein may have a measure of Zn2+ made soluble from about 20 mg/L to about 750 mg/L. In some embodiments, a working solution of a bioreactor system described herein may have a measure of Zn2+ made soluble from about 20 mg/L to about 50 mg/L, about 20 mg/L to about 100 mg/L, about 20 mg/L to about 150 mg/L, about 20 mg/L to about 200 mg/L, about 20 mg/L to about 250 mg/L, about 20 mg/L to about 300 mg/L, about 20 mg/L to about 350 mg/L, about 20 mg/L to about 400 mg/L, about 20 mg/L to about 450 mg/L, about 20 mg/L to about 500 mg/L, about 20 mg/L to about 750 mg/L, about 50 mg/L to about 100 mg/L, about 50 mg/L to about 150 mg/L, about 50 mg/L to about 200 mg/L, about 50 mg/L to about 250 mg/L, about 50 mg/L to about 300 mg/L, about 50 mg/L to about 350 mg/L, about 50 mg/L to about 400 mg/L, about 50 mg/L to about 450 mg/L, about 50 mg/L to about 500 mg/L, about 50 mg/L to about 750 mg/L, about 100 mg/L to about 150 mg/L, about 100 mg/L to about 200 mg/L, about 100 mg/L to about 250 mg/L, about 100 mg/L to about 300 mg/L, about 100 mg/L to about 350 mg/L, about 100 mg/L to about 400 mg/L, about 100 mg/L to about 450 mg/L, about 100 mg/L to about 500 mg/L, about 100 mg/L to about 750 mg/L, about 150 mg/L to about 200 mg/L, about 150 mg/L to about 250 mg/L, about 150 mg/L to about 300 mg/L, about 150 mg/L to about 350 mg/L, about 150 mg/L to about 400 mg/L, about 150 mg/L to about 450 mg/L, about 150 mg/L to about 500 mg/L, about 150 mg/L to about 750 mg/L, about 200 mg/L to about 250 mg/L, about 200 mg/L to about 300 mg/L, about 200 mg/L to about 350 mg/L, about 200 mg/L to about 400 mg/L, about 200 mg/L to about 450 mg/L, about 200 mg/L to about 500 mg/L, about 200 mg/L to about 750 mg/L, about 250 mg/L to about 300 mg/L, about 250 mg/L to about 350 mg/L, about 250 mg/L to about 400 mg/L, about 250 mg/L to about 450 mg/L, about 250 mg/L to about 500 mg/L, about 250 mg/L to about 750 mg/L, about 300 mg/L to about 350 mg/L, about 300 mg/L to about 400 mg/L, about 300 mg/L to about 450 mg/L, about 300 mg/L to about 500 mg/L, about 300 mg/L to about 750 mg/L, about 350 mg/L to about 400 mg/L, about 350 mg/L to about 450 mg/L, about 350 mg/L to about 500 mg/L, about 350 mg/L to about 750 mg/L, about 400 mg/L to about 450 mg/L, about 400 mg/L to about 500 mg/L, about 400 mg/L to about 750 mg/L, about 450 mg/L to about 500 mg/L, about 450 mg/L to about 750 mg/L, or about 500 mg/L to about 750 mg/L.

Population of the microbe may be measured using methods including but not limited to spectrophotometers, cell counting, measures of turbidity, hemocytometers, electronic enumeration, measures of cell mass, measures of cell activity, CFU morphology on general nutrient media or selective and/or differentiating media, PCR, semi-quantitative PCR, and qPCR.

Zinc-solubilizing microbes may be identified using methods using agar plates or liquid testing solutions. Microbes may be tested on agar plates with insoluble zinc and the measurement of zinc solubilization capacity (e.g., amount of zinc made soluble), usually by the diameter of the zone of clearing around the microbial colony on the agar plate after incubation, may classify a microbe as capable of zinc solubilization. The greater the diameter, the more zinc was solubilized by the microbial colony. Microbes may be tested in liquid solution containing insoluble zinc to measure zinc solubilization capacity, by measuring the zinc made soluble after incubation. Microbes are inoculated in liquid zinc-media containing an insoluble source of zinc. After incubation, the solubilized zinc is measured in the form of Zn2+using instruments such as Inductively Couples Plasma (ICP) Atomic Emission Spectrometer.

The addition of the target isolate (e.g., the inoculum of the microbial strain/microbe) can have an added benefit in improving target functionality of working solution in a reactor of the bioreactor system and/or in the output base product of the bioreactor system. In some embodiments, addition of the inoculum of the microbial strain can improve the zinc solubilization capacity of working fluids and base product of a bioreactor system compared to working fluids and base product of a bioreactor system without an inoculum of the microbial strain.

In some embodiments, a working solution and/or base product of a bioreactor system inoculated with a target isolate may have an at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, or greater than 80% increase in Zn2+ concentration in comparison with that of a working solution and/or base product of a bioreactor system not inoculated with a target isolate. In some embodiments, a working solution and/or base product of a bioreactor system inoculated with a target isolate may have an at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15% or less than about 15% increase in Zn2+ concentration in comparison with that of a working solution and/or base product of a bioreactor system not inoculated with a target isolate.

In some embodiments, a working solution and/or base product of a bioreactor system inoculated with a target isolate may have from about 10% to about 80% increase in Zn2+ concentration in comparison with that of a working solution and/or base product of a bioreactor system not inoculated with a target isolate. In some embodiments, a working solution and/or base product of a bioreactor system inoculated with a target isolate may have from about 10% to about 15%, about 10% to about 20%, about 10% to about 25%, about 10% to about 30%, about 10% to about 35%, about 10% to about 40%, about 10% to about 45%, about 10% to about 50%, about 10% to about 60%, about 10% to about 70%, about 10% to about 80%, about 15% to about 20%, about 15% to about 25%, about 15% to about 30%, about 15% to about 35%, about 15% to about 40%, about 15% to about 45%, about 15% to about 50%, about 15% to about 60%, about 15% to about 70%, about 15% to about 80%, about 20% to about 25%, about 20% to about 30%, about 20% to about 35%, about 20% to about 40%, about 20% to about 45%, about 20% to about 50%, about 20% to about 60%, about 20% to about 70%, about 20% to about 80%, about 25% to about 30%, about 25% to about 35%, about 25% to about 40%, about 25% to about 45%, about 25% to about 50%, about 25% to about 60%, about 25% to about 70%, about 25% to about 80%, about 30% to about 35%, about 30% to about 40%, about 30% to about 45%, about 30% to about 50%, about 30% to about 60%, about 30% to about 70%, about 30% to about 80%, about 35% to about 40%, about 35% to about 45%, about 35% to about 50%, about 35% to about 60%, about 35% to about 70%, about 35% to about 80%, about 40% to about 45%, about 40% to about 50%, about 40% to about 60%, about 40% to about 70%, about 40% to about 80%, about 45% to about 50%, about 45% to about 60%, about 45% to about 70%, about 45% to about 80%, about 50% to about 60%, about 50% to about 70%, about 50% to about 80%, about 60% to about 70%, about 60% to about 80%, or about 70% to about 80% increase in Zn2+ concentration in comparison with that of a working solution and/or base product of a bioreactor system not inoculated with a target isolate.

A bioreactor system may be inoculated with a microbial strain at the start of a bioreactor system, which may allow for the microbial strain to flow through the system and working fluids of the reactors. Without wishing to be bound by theory, a bioreactor system inoculated with a microbial strain may increase a zinc solubilization capacity of a working fluid and/or a base product compared to a working fluid and/or base product of an otherwise identical bioreactor system with no inoculated microbial strain. Without wishing to be bound by theory, a bioreactor system inoculated with a microbial strain may increase a zinc solubilization capacity of a working fluid and/or a base product compared to a working fluid and/or base product of an otherwise identical bioreactor system with the microbial strain added (e.g., spiked) at the end of the system. Without wishing to be bound by theory, a bioreactor system inoculated with a microbial strain may increase a zinc solubilization capacity of a base product compared to a base product of an otherwise identical bioreactor system with the microbial strain added (e.g., spiked) at the end of the system. Incubation of the microbial strain in the bioreactor system may enrich the working fluid with zinc-solubilizing microbes and/or generate zinc-solubilizing metabolites.

### B. Phosphate Solubilizer Microbial Isolates

Phosphorus is an essential macronutrient for plant growth and development and phosphate deficiencies can impair the physiological and biochemical functioning of plants. Phosphorus availability is important for growth promoting properties such as increasing photosynthesis, biomass of shoots and roots, strengthening the stems, increasing leafage and leaf area, formation of flowers and seeds, energy production, nitrogen fixation, resistance to infections and disease, and transformation of sugar to starch. Although most soils contain considerable amounts of phosphorus, the availability of soluble phosphorus is limited because of fixation as insoluble phosphates of aluminum, calcium, and iron, and other complexes.

In some embodiments, desirable microbes may have phosphate-solubilizing properties (e.g., enhancing bioavailability of phosphate, increasing phosphate uptake, or any combination thereof). The other desirable microbes may be of the bacterial species Bacillus safensis, Bacillus amyloliquefaciens, Bacillus licheniformis, Bacillus megaterium, Commonas testosteroni, Enterobacter cloacae, Citrabacter freundii, Pseudomonas spp., Agrobacterium spp., Bacillus circulans, or any combination thereof.

In some embodiments, operating a bioreactor system disclosed herein provides a synergistic effect in the production of a biostimulant composition. In some embodiments, a bioreactor system that comprises an established and/or stable population of a phosphate-solubilizing microbial strain (e.g., one or more of the phosphate-solubilizing strains disclosed herein and/or related strains) produces a biostimulant having a greater phosphate solubilization effect than could be obtained from an otherwise similar bioreactor system that does not have the established population of the phosphate-solubilizing strain. The phosphate-solubilizing effect of the biostimulant produced according to methods disclosed herein may be greater than a biostimulant produced in an otherwise similar system lacking the established population of the phosphate-solubilizing strain, even if the latter biostimulant has one or more of the phosphate-solubilizing strains added to it after it is produced. For example, a biostimulant composition produced according to the methods disclosed herein may have greater capacity to increase phosphate uptake compared to a biostimulant composition produced by an otherwise similar system lacking the established population of the phosphate-solubilizing strain. For example, a biostimulant composition produced according to the methods disclosed herein may have greater phosphate-solubilizing effects compared to a biostimulant composition produced by an otherwise similar system lacking the established population of the phosphate-solubilizing strain. For example, a biostimulant composition produced according to the methods disclosed herein may have greater capacity to increase soil organic phosphate and mineralization and uptake of organic phosphate (e.g., organically-bound phosphate) compared to that of a biostimulant composition produced by an otherwise similar system lacking the established population of the phosphate-solubilizing strain. Without being bound by theory, the presence of the target phosphate-solubilizing strain population in the bioreactor system may promote production of phosphate-solubilizing digestion products in the bioreactor system, which are present in the product.

Phosphate solubilizing microorganisms may be capable of hydrolyzing organic and inorganic insoluble phosphorus compounds to soluble phosphate that plants can uptake. Phosphate solubilizing microorganisms can include, but are not limited to, strains from bacteria (Bacillus, Pseudomonas, and Rhizobium), arbuscular mycorrhizal, fungi (Penicillium and Aspergillus), and actinomycetes. In some embodiments, phosphate solubilizing bacteria may comprise strains of Bacillus safensis, Bacillus amyloliquefaciens, Bacillus licheniformis, Bacillus megaterium, Commonas testosteroni, Enterobacter cloacae, Citrabacter freundii, Pseudomonas spp., Agrobacterium spp., Bacillus circulans, or any combination thereof. Mechanisms of phosphate solubilization can include, but are not limited to, organic mineralization, chelation, and lowering soil pH levels. Without wishing to be bound by theory, it is believed that lower pH levels leads to a decrease in fixed phosphorus by calcium, aluminum, and iron, and hence greater phosphate solubilization. In some embodiments, the pH is between 3 and 8.5 after the addition of phosphate solubilizing bacteria. In some embodiments, the pH is lower than 5 after the addition of phosphate solubilizing bacteria.

In some embodiments, disclosed herein are phosphate solubilizing microbial strains, including compositions that comprise such strains and methods of using such strains to promote plant growth.

In some embodiments, the microbial strain is from a Bacillus genus. In some embodiments, the microbial strain is from a Bacillus amyloliquefaciens species. In some embodiments, the microbial strain is from a bacterial species other than Bacillus amyloliquefaciens. In some embodiments, the Bacillus amyloliquefaciens strain is deposited under Accession No. PTA-127657, or an isolated clone thereof. In some embodiments, the 16S rRNA gene of the microbial strain comprises a nucleotide sequence selected from SEQ ID NO.: 10. In some embodiments, the microbial strain comprises a 16S rRNA gene comprising a nucleotide sequence of SEQ ID NO.: 10. In some embodiments, the microbial strain comprises a 16S rRNA gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to the nucleotide sequences as set forth in SEQ ID NO.: 10. In some embodiments, the 16S rRNA gene of the microbial strain comprises a nucleotide sequence that exhibits at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to any one of the nucleotide sequences as set forth in SEQ ID NO.: 10. In some embodiments, the gyrB gene of the microbial strain comprises the nucleotide sequence of SEQ ID NO: 12. In some embodiments, the microbial strain comprises a gyrB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% sequence identity to a nucleotide sequence as set forth in SEQ ID NO: 12. In some embodiments, the rpoB gene of the microbial strain comprises the nucleotide sequence of SEQ ID NO: 14. In some embodiments, the microbial strain comprises a rpoB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% sequence identity to a nucleotide sequence as set forth in SEQ ID NO: 14. Some embodiments provide a genus of plant growth-promoting microorganisms comprising any of the DNA sequences described herein and which enhances the health, growth and/or yield of a plant, as described herein.

In some embodiments, the microbial strain is from a Bacillus genus. In some embodiments, the microbial strain is from a Bacillus licheniformis species. In some embodiments, the microbial strain is from a bacterial species other Bacillus licheniformis. In some embodiments, the Bacillus licheniformis strain is deposited under Accession No. PTA-127656, or an isolated clone thereof. In some embodiments, the 16S rRNA gene of the microbial strain comprises a nucleotide sequence selected from SEQ ID NO.: 11. In some embodiments, the microbial strain comprises a 16S rRNA gene comprising a nucleotide sequence of SEQ ID NO.: 11. In some embodiments, the microbial strain comprises a 16S rRNA gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to any one of the nucleotide sequences as set forth in SEQ ID NO.: 11. In some embodiments, the 16S rRNA gene of the microbial strain comprises a nucleotide sequence that exhibits at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to any one of the nucleotide sequences as set forth in SEQ ID NO.: 11. In some embodiments, the gyrB gene of the microbial strain comprises the nucleotide sequence of SEQ ID NO: 13. In some embodiments, the microbial strain comprises a gyrB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% sequence identity to a nucleotide sequence as set forth in SEQ ID NO: 13. In some embodiments, the rpoB gene of the microbial strain comprises the nucleotide sequence of SEQ ID NO: 15. In some embodiments, the microbial strain comprises a rpoB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or 100% sequence identity to a nucleotide sequence as set forth in SEQ ID NO: 15. Some embodiments provide a genus of plant growth-promoting microorganisms comprising any of the DNA sequences described herein and which enhances the health, growth and/or yield of a plant, as described herein.

Chemical analysis of root exudates from plants treated with base product from the PST-sIP system (e.g., PST supernatant containing phosphate solubilizing bacteria) may indicate roots exudate organic acids. The metabolites of the PST base product may affect the chemistry of the root exudates. Filter-sterilized PST may affect the chemistry of the root exudates. In some embodiments, the organic acids comprise lactic acid, succinic acid, propionic acid, 2-ketogluconic acid, ketoglutaric acid, itaconic acid, isovaleric acid, isobutyric acid, acetic acid, oxalic acid, citric acid, malic acid, glyoxalic acid, fumaric acid, tartaric acid, a-ketobutyric acid, malonic acid, glycolic acid, glutamic acid, maleic acid, aspartic acid, gluconic acid, or a combination thereof. In some embodiments, the organic acids comprise lactic acid, succinic acid, malic acid, ketoglutaric acid, citric acid, or a combination thereof. In some embodiments, the released organic acids can increase solubilization of phosphates.

TABLE 4
Exemplary 16S rRNA Sequences of Bacterial Isolates

| SEQ ID | | |
|---|---|---|
| NO. | Strain | Sequence |
| 10 | MS2839 | GAGAGTTTGATCCTGGCTCAGGACGAACGCTGGCGGCGTGCCTAATACATGCAAGTCGA |
| | | GCGGACAGATGGGAGCTTGCTCCCTGATGTTAGCGGCGGACGGGTGAGTAACACGTGGG |
| | | TAACCTGCCTGTAAGACTGGGATAACTCCGGGAAACCGGGGCTAATACCGGATGGTTGT |
| | | YTGAACCGCATGGTTCAGACATAAAAGGTGGCTTYGGCTACCACTTACAGATGGACCCG |
| | | CGGCGCATTAGCTAGTTGGTGAGGTAACGGCTCACCAAGGCRACGATGCGTAGCCGACC |
| | | TGAGAGGGTGATCGGCCACACTGGGACTGAGACACGGCCCAGACTCCTACGGGAGGCAG |
| | | CAGTAGGGAATCTTCCGCAATGGACGAAAGTCTGACGGAGCAACGCCGCGTGAGTGATG |
| | | AAGGTTTTCGGATCGTAAAGCTCTGTTGTTAGGGAAGAACAAGTGCCGTTCAAATAGGG |
| | | CGGCACCTTGACGGTACCTAACCAGAAAGCCACGGCTAACTACGTGCCAGCAGCCGCGG |
| | | TAATACGTAGGTGGCAAGCGTTGTCCGGAATTATTGGGCGTAAAGGGCTCGCAGGCGGT |
| | | TTCTTAAGTCTGATGTGAAAGCCCCCGGCTCAACCGGGGAGGGTCATTGGAAACTGGGG |
| | | AACTTGAGTGCAGAAGAGGAGAGTGGAATTCCACGTGTAGCGGTGAAATGCGTAGAGAT |
| | | GTGGAGGAACACCAGTGGCGAAGGCGACTCTCTGGTCTGTAACTGACGCTGAGGAGCGA |
| | | AAGCGTGGGGAGCGAACAGGATTAGATACCCTGGTAGTCCACGCCGTAAACGATGAGTG |
| | | CTAAGTGTTAGGGGGTTTCCGCCCCTTAGTGCTGCAGCTAACGCATTAAGCACTCCGCC |
| | | TGGGGAGTACGGTCGCAAGACTGAAACTCAAAGGAATTGACGGGGGCCCGCACAAGCGG |
| | | TGGAGCATGTGGTTTAATTCGAAGCAACGCGAAGAACCTTACCAGGTCTTGACATCCTC |
| | | TGACAATCCTAGAGATAGGACGTCCCCTTCGGGGGCAGAGTGACAGGTGGTGCATGGTT |
| | | GTCGTCAGCTCGTGTCGTGAGATGTTGGGTTAAGTCCCGCAACGAGCGCAACCCTTGAT |
| | | CTTAGTTGCCAGCATTCAGTTGGGCACTCTAAGGTGACTGCCGGTGACAAACCGGAGGA |
| | | AGGTGGGGATGACGTCAAATCATCATGCCCCTTATGACCTGGGCTACACACGTGCTACA |
| | | ATGGACAGAACAAAGGGCAGCGAAACCGCGAGGTTAAGCCAATCCCACAAATCTGTTCT |
| | | CAGTTCGGATCGCAGTCTGCAACTCGACTGCGTGAAGCTGGAATCGCTAGTAATCGCGG |
| | | ATCAGCATGCCGCGGTGAATACGTTCCCGGGCCTTGTACACACCGCCCGTCACACCACG |
| | | AGAGTTTGTAACACCCGAAGTCGGTGAGGTAACCTTTTATGGAGCCAGCCGCCGAAGGT |
| | | GGGACAGATGATTGGGGTGAAGTCGTAACAAGGTAGCCGTATCGGAAGGTGCGGCTGGA |
| | | TCACCTCCTTTCTA |
| 11 | MS1835 | AGAGTTTGATCCTGGCTCAGGACGAACGCTGGCGGCGTGCCTAATACATGCAAGTCGAG |
| | | CGGACCGAYGGGAGCTTGCTCCCTTAGGTCAGCGGCGGACGGGTGAGTAACACGTGGGT |
| | | AACCTGCCTGTAAGACTGGGATAACTCCGGGAAACCGGGGCTAATACCGGATGCTTGAT |
| | | TGAACCGCATGGTTCCAATCATAAAAGGTGGCTTTTAGCTACCACTTRCAGATGGACCC |
| | | GCGGCGCATTAGCTAGTTGGTGAGGTAACGGCTCACCAAGGCGACGATGCGTAGCCGAC |
| | | CTGAGAGGGTGATCGGCCACACTGGGACTGAGACACGGCCCAGACTCCTACGGGAGGCA |
| | | GCAGTAGGGAATCTTCCGCAATGGACGAAAGTCTGACGGAGCAACGCCGCGTGAGTGAT |
| | | GAAGGTTTTCGGATCGTAAAACTCTGTTGTTAGGGAAGAACAAGTACCGTTCGAATAGG |
| | | GCGGYACCTTGACGGTACCTAACCAGAAAGCCACGGCTAACTACGTGCCAGCAGCCGCG |
| | | GTAATACGTAGGTGGCAAGCGTTGTCCGGAATTATTGGGCGTAAAGCGCGCGCAGGCGG |
| | | TTTCTTAAGTCTGATGTGAAAGCCCCCGGCTCAACCGGGGAGGGTCATTGGAAACTGGG |
| | | GAACTTGAGTGCAGAAGAGGAGAGTGGAATTCCACGTGTAGCGGTGAAATGCGTAGAGA |
| | | TGTGGAGGAACACCAGTGGCGAAGGCGACTCTCTGGTCTGTAACTGACGCTGAGGCGCG |
| | | AAAGCGTGGGGAGCGAACAGGATTAGATACCCTGGTAGTCCACGCCGTAAACGATGAGT |
| | | GCTAAGTGTTAGAGGGTTTCCGCCCTTTAGTGCTGCAGCAAACGCATTAAGCACTCCGC |
| | | CTGGGGAGTACGGTCGCAAGACTGAAACTCAAAGGAATTGACGGGGGCCCGCACAAGCG |
| | | GTGGAGCATGTGGTTTAATTCGAAGCAACGCGAAGAACCTTACCAGGTCTTGACATCCT |
| | | CTGACAACCCTAGAGATAGGGCTTCCCCTTCGGGGGCAGAGTGACAGGTGGTGCATGGT |
| | | TGTCGTCAGCTCGTGTCGTGAGATGTTGGGTTAAGTCCCGCAACGAGCGCAACCCTTGA |
| | | TCTTAGTTGCCAGCATTCAGTTGGGCACTCTAAGGTGACTGCCGGTGACAAACCGGAGG |
| | | AAGGTGGGGATGACGTCAAATCATCATGCCCCTTATGACCTGGGCTACACACGTGCTAC |
| | | AATGGGCAGAACAAAGGGCAGCGAAGCCGCGAGGCTAAGCCAATCCCACAAATCTGTTC |
| | | TCAGTTCGGATCGCAGTCTGCAACTCGACTGCGTGAAGCTGGAATCGCTAGTAATCGCG |
| | | GATCAGCATGCCGCGGTGAATACGTTCCCGGGCCTTGTACACACCGCCCGTCACACCAC |
| | | GAGAGTTTGTAACACCCGAAGTCGGTGAGGTAACCTTTTGGAGCCAGCCGCCGAAGGTG |
| | | GGACAGATGATTGGGGTGAAGTCGTAACAAGGTAGCCGTATCGGAAGGTGCGGCTGGAT |
| | | CACCTCCTTTCA |

TABLE 5
Exemplary gyrB Gene Sequences of Bacterial Isolates

| SEQ ID | | |
|---|---|---|
| NO. | Strain | Sequence |
| 12 | MS2839 | ATGTACATCGGATCAACTAACAGCAAAGGCCTTCACCACTTGGTGTGGGAAATCGTCGA |
| | | CAACAGTATTGACGAAGCCCTGGCCGGTTATTGTACAGATATTAACATCGAGATTGAAA |
| | | AAGATAACAGCATTACCGTTAAGGACAACGGGCGCGGAATTCCGGTCGGTATCCAGGAG |
| | | AAGATGGGCCGCCCTGCGGTTGAAGTCATCATGACCGTTCTCCACGCCGGCGGTAAATT |
| | | TGACGGAAGCGGATATAAAGTATCCGGCGGTCTTCACGGTGTAGGGGCATCTGTCGTAA |
| | | ACGCCTTGTCGACTACTCTTGACGTTACGGTTCATCGTGACGGAAAAATCCACTATCAG |
| | | GCGTACGAGCGCGGTGTACCTGTGGCCGATCTTGAAGTGATCGGTGATACTGATAAGAC |
| | | CGGAACGATTACGCACTTCGTTCCGGATCCGGAAATCTTCAAAGAAACAACCGTATACG |
| | | ACTATGATCTGCTTTCAAACCGTGTCCGGGAATTGGCCTTCCTGACAAAAGGCGTAAAC |
| | | ATCACGATTGAAGACAAACGTGAAGGACAAGAACGGAAAAACGAGTACCACTACGAAGG |
| | | CGGAATCAAAAGCTATGTTGAGTACTTAAACCGTTCCAAAGAAGTCGTTCATGAAGAGC |
| | | CGATTTATATCGAAGGCGAGAAAGACGGCATAACGGTTGAAGTTGCATTGCAATACAAC |
| | | GACAGCTATACAAGCAATATTTATTCTTTCACGAATAATATCAACACATACGAAGGCGG |
| | | CACGCACGAGGCCGGATTTAAAACCGGTCTGACCCGTGTCATAAACGACTATGCAAGAA |
| | | GAAAAGGGATTTTCAAAGAAAATGATCCGAATTTAAGCGGGGATGATGTGAGAGAAGGG |
| | | CTGACTGCCATTATTTCAATTAAGCACCCTGATCCGCAATTCGAAGGGCAGACGAAAAC |
| | | GAAGCTCGGCAACTCCGAAGCGAGAACGATCACTGATACGCTGTTTTCTTCTGCGCTGG |
| | | AAACATTCCTTCTTGAAAATCCGGACTCAGCCCGCAAAATCGTTGAAAAAGGTTTAATG |
| | | GCCGCAAGAGCGCGGATGGCAGCGAAAAAAGCACGGGAATTGACCCGGCGCAAAAGTGC |
| | | GCTTGAGATTTCCAATCTGCCGGGCAAACTGGCGGACTGTTCTTCTAAAGATCCGAGCA |
| | | TTTCCGAGCTGTATATCGTAGAGGGTGACTCTGCGGGCGGATCAGCGAAACAGGGACGG |
| | | GACCGTCATTTCCAAGCTATTCTGCCGCTGCGCGGTAAGATTCTGAACGTTGAGAAAGC |
| | | CAGACTTGATAAGATTCTCTCAAACAATGAGGTCAGATCAATGATCACGGCCCTCGGAA |
| | | CAGGAATCGGAGAAGATTTTAATCTTGAAAAAGCGCGTTATCATAAAGTGGTCATCATG |
| | | ACGGATGCAGATGTTGACGGCGCCCACATCAGAACGCTTTTATTAACGTTCTTCTACAG |
| | | ATACATGCGGGAAATCATCGAAAACGGCTATGTCTACATTGCCCAGCCGCCGCTTTATA |
| | | AAGTGCAGCAGGGAAAACGGGTGGAATACGCTTATAACGATAAGCAGCTTGATGAGCTG |
| | | TTAAAAGAGCTTCCGCAATCACCTAAGCCTGGCCTCCAGCGTTATAAAGGTCTTGGAGA |
| | | AATGAACGCGACTCAGCTTTGGGAAACGACAATGGACCCTGCGACCAGAACGCTTCTGC |
| | | AAGTCAATCTTGAAGATGCAATGGACGCTGACGAGACTTTTGAAATGCTGATGGGTGAC |
| | | AAAGTAGAACCGCGGAGAAACTTCATAGAAGCAAACGCCAGATACGTGAAAAATCTTGA |
| | | TATTTAA |
| 13 | MS1835 | GTGGCAATGGAACAGCAAAATAATTACGATGAAAATCAGATACAGGTTCTAGAGGGATT |
| | | GGAAGCCGTTCGGAAAAGACCCGGTATGTACATCGGGTCGACAAGCGGCAAAGGTCTGC |
| | | ACCATTTAGTATGGGAAATTGTCGATAACAGCATCGATGAAGCATTGGCCGGTTACTGC |
| | | ACTGAAATCAATGTCGCAATTGAAAAAGACAACAGCATCACAGTAAAAGACAACGGACG |
| | | GGGTATCCCGGTCGGTATTCATGAGAAGATGGGTCGTCCCGCTGTGGAAGTCATCATGA |
| | | CTGTCCTGCACGCCGGAGGAAAGTTTGACGGAAGCGGATATAAAGTTTCGGGCGGTTTG |
| | | CACGGCGTCGGTGCTTCTGTTGTTAACGCCCTTTCAACCGAGCTGGATGTAACGGTTTA |
| | | CAGAGATGGAAAAATCCATTATCAGGAATTTGAACGTGGCGTTCCGAAAGCTGATTTGA |
| | | AAGTCATTGGAGATACGGAAGTGACGGGAACGACCACACACTTCAAGCCTGATCCGGAA |
| | | ATATTCACGGAAACGACTGAATACGACTATGATACGCTCGCCACTCGTGTCCGCGAACT |
| | | CGCTTTCTTGACAAAAGGCGTCAAAATCACGATCGAAGACAAGCGAGAAGGAAAAGAAC |
| | | GCAAGAATGAATACTGCTATGAAGGCGGTATTAAAAGCTATGTTGAACACTTGAACCGT |
| | | TCGCGGGAAGTTATTCATGAAGAGCCGGTCTATATTGAAGGATCCAAAGACGGCATTAC |
| | | AGTCGAGGTGGCTCTTCAATACAATGACAGCTATACAAGCAACATTTATTCATTTGCTA |
| | | ACAACATTCATACGTATGAAGGCGGAACCCATGAAGCCGGCTTTAAGACCGGTTTGACG |
| | | AGGGTCATCAATGATTACGCGAGAAGAAACGGCGTATTCAAAGAAAGCGATCCGAACTT |
| | | AAGCGGAGAAGACGTCCGGGAAGGTTTGACAGCGATTATTTCAATCAAGCACCCGGATC |
| | | CTCAATTTGAAGGGCAGACGAAAACAAAGCTCGGCAACTCAGAAGCGCGTACGATAACA |
| | | GATGCGCTATTTTCAGAAGCGCTTGAAAAGTTTCTGCTAGAAAACCCGGATTCAGCGAA |
| | | AAAAATCGTTGAAAAAGGGGTTATGGCCGCCAGAGCACGGATGGCTGCAAAGAAAGCAC |
| | | GCGAATTGACGCGCAGAAAAAGCGCCCTTGAAGTGTCCAATCTGCCGGGGAAACTTGCT |
| | | GACTGTTCTTCTAAAGACCCGACGATTTCCGAACTTTACATCGTTGAGGGTGACTCTGC |
| | | GGGCGGATCGGCAAAACAGGGCCGCGACCGTCATTTCCAAGCAATTTTGCCTTTGAGAG |
| | | GGAAAATTTTGAACGTCGAAAAAGCCCGCCTGGACAAAATATTGTCCAACAATGAGGTT |
| | | CGTTCTATGATCACCGCCCTTGGCACCGGAATCGGGGAAGATTTCAACCTTGAAAAAGC |
| | | CCGCTACCACAAAGTCGTGATTATGACGGATGCCGACGTCGATGGTGCACACATCAGAA |
| | | CATTGCTGCTGACCTTTTTCTACCGCTACATGAGACAGATTATCGAACAAGGATATGTC |
| | | TACATCGCTCAGCCGCCTCTTTACAAAGTACAGCAGGGGAAACGCGTTGAGTATGCGTA |
| | | CAATGACCAGCAGCTCGAAGAAATTTTAAAAGAGCTTCCGCAAAATCCTAAGCCAGGCC |
| | | TGCAGCGCTACAAAGGTTTGGGCGAGATGAATGCGACACAGCTTTGGGAAACGACGATG |
| | | GATCCAGAAACGCGGACATTGCTTCAAGTAACGCTCAAAGATGCGATTGATGCGGATGA |
| | | AACCTTTGAAATGCTGATGGGCGACAAGGTTGAACCGCGCCGCAACTTCATCGAAGAGA |
| | | ATGCCCGATATGTAAAAAATTTGGATATTTAA |

TABLE 6
Exemplary rpoB Gene Sequences of Bacterial Isolates

| SEQ ID | | |
|---|---|---|
| NO. | Strain | Sequence |
| 14 | MS2839 | ATGGGTGATTTCCCTATTATGACAGATACCGGTACTTTTATCATCAACGGTGCAGAACG |
| | | TGTTATCGTATCTCAGCTTGTTCGGTCTCCAAGTGTATATTTCAGTGGTAAAGTAGACA |
| | | AAAACGGTAAAAAAGGTTTTACCGCGACTGTCATTCCAAACCGTGGCGCATGGTTAGAA |
| | | TACGAAACTGATGCGAAAGATGTTGTGTATGTCCGCATTGATCGCACACGTAAGTTGCC |
| | | GGTTACGGTTCTTTTGCGTGCTCTCGGCTTCGGTTCCGACCAAGAGATTCTCGATCTCA |
| | | TTGGTGAGAACGAATATCTCCGCAATACACTGGATAAGGACAACACTGAAAACAGTGAC |
| | | AAAGCGCTTCTTGAAATCTATGAGCGCCTTCGTCCCGGAGAGCCGCCTACAGTAGAAAA |
| | | CGCAAAAAGCTTGCTGGATTCCCGCTTCTTTGATCCGAAGCGATACGATCTTGCGAATG |
| | | TAGGACGCTATAAAATTAATAAAAAGCTTCATATCAAGAACCGCCTGTTTAACCAGCGC |
| | | CTTGCAGAAACACTGGTGGATCCGGAAACCGGTGAAATTCTCGCTGAAAAAGGGCAGAT |
| | | TCTTGACAGAAGAACACTTGATAAAGTACTGCCATACTTAGAAAATGGAATCGGCTTCA |
| | | GAAAACTTTATCCTAACGGCGGCGTCGTCGAGGATGAAGTGATGCTTCAATCCATTAAA |
| | | ATCTATGCTCCTACCGATGCAGAAGGAGAGCAGACGATCAATGTGATCGGCAATGCTTA |
| | | CATCGAAGAGGCGATTAAAAACATTACGCCTGCTGATATTATTTCTTCTATCAGCTACT |
| | | TCTTCAACCTCCTGCACGGAGTGGGCGACACTGATGATATCGACCATCTCGGAAACCGC |
| | | CGTCTGCGTTCTGTAGGTGAGCTCCTGCAAAACCAATTCCGTATCGGTTTAAGCCGGAT |
| | | GGAACGTGTCGTACGTGAAAGAATGTCTATTCAAGACACGAATACAATTACGCCGCAGC |
| | | AGCTGATTAACATCAGACCTGTTATTGCGTCTATTAAAGAGTTCTTCGGAAGCTCACAG |
| | | CTTTCTCAATTCATGGATCAGACGAACCCGCTTGCTGAATTGACGCACAAACGCCGTCT |
| | | GTCAGCTCTCGGACCGGGCGGTTTGACACGTGAGCGCGCAGGTATGGAAGTGCGTGACG |
| | | TTCACTACTCTCACTATGGCCGTATGTGTCCGATTGAAACGCCTGAGGGCCCGAACATC |
| | | GGTTTGATCAACTCATTGTCATCATTTGCGAAAGTAAACCGCTTTGGTTTCATTGAGAC |
| | | GCCATACCGCCGCGTTGATCCTGAAACAGGAAAAGTAACGCCTAGAATCGACTACCTGA |
| | | CTGCTGATGAAGAGGATAACTATGTCGTAGCCCAAGCGAATGCTAAGCTGAGCGATGAC |
| | | GGTTCTTTCTTGGATGACAGCATCGTAGCGCGTTTCAGAGGGGAAAACACCGTTGTAGC |
| | | CCGCAACCGCGTGGATTACATGGACGTATCTCCTAAACAGGTTGTATCTGCTGCGACAG |
| | | CATGTATTCCGTTCTTGGAAAACGATGACTCGAACCGCGCCCTCATGGGAGCGAACATG |
| | | CAGCGTCAGGCTGTGCCTTTGATGCAGCCGGAAGCTCCGATCGTCGGAACGGGTATGGA |
| | | ATACGTATCCGGTAAAGACTCCGGTGCAGCCGTTATTTGTAAACACCCTGGTATCGTTG |
| | | AACGGGTGGAAGCGAAAAACGTATGGGTGCGCCGCTATGAAGAAATTGACGGCCAAAAA |
| | | GTAAAAGGCAACCTGGATAAGTACAGCTTGCTGAAATTTGTCCGCTCCAACCAAGGTAC |
| | | GTGCTACAACCAGCGTCCGATCGTCAGTGTCGGCGATGAAGTAGTCAAAGGAGAAATCC |
| | | TTGCTGACGGACCTTCAATGGAGCTTGGTGAACTTGCTCTCGGCCGCAACGTAATGGTC |
| | | GGCTTCATGACATGGGATGGTTACAACTATGAGGATGCCATCATCATGAGTGAACGCCT |
| | | TGTGAAAGATGATGTATACACATCTATTCACATTGAAGAATATGAATCAGAAGCACGTG |
| | | ATACAAAGCTTGGGCCGGAAGAGATCACCCGCGATATTCCAAACGTAGGGGAAGACGCG |
| | | CTTCGCAATCTTGATGACCGCGGAATTATCCGTATCGGTGCGGAAGTCAACGACGGAGA |
| | | CCTTCTCGTAGGTAAAGTAACGCCTAAAGGTGTAACTGAGCTTACGGCTGAAGAACGCC |
| | | TTCTGCATGCGATCTTTGGAGAAAAAGCGCGTGAAGTCCGTGATACTTCTCTCCGTGTG |
| | | CCTCACGGCGGCGGCGGAATTATCCACGACGTAAAAGTCTTCAACCGTGAAGACGGCGA |
| | | CGAACTTCCTCCGGGAGTGAACCAGCTTGTACGCGTATATATCGTTCAGAAACGTAAGA |
| | | TTTCTGAAGGTGATAAAATGGCCGGACGTCACGGAAACAAAGGGGTTATCTCGAAGATT |
| | | CTTCCTGAAGAAGATATGCCTTACCTTCCTGACGGCACGCCGATCGATATCATGCTTAA |
| | | CCCGCTGGGTGTACCATCACGTATGAATATCGGTCAGGTATTAGAACTTCACATGGGTA |
| | | TGGCTGCCCGCTACCTCGGCATTCACATCGCGTCACCTGTATTTGACGGCGCGCGTGAA |
| | | GAAGATGTGTGGGAAACACTTGAAGAAGCAGGCATGTCAAGAGACGCTAAAACAGTACT |
| | | TTATGACGGCCGTACGGGAGAACCGTTTGACAACCGTGTATCTGTCGGAATCATGTACA |
| | | TGATCAAACTGGCGCACATGGTTGATGATAAACTTCATGCCCGTTCTACAGGTCCTTAC |
| | | TCACTTGTTACGCAGCAGCCTCTCGGCGGTAAAGCCCAATTCGGCGGACAGCGTTTCGG |
| | | TGAGATGGAGGTTTGGGCGCTTGAAGCTTACGGCGCAGCTTACACGCTTCAAGAAATCC |
| | | TGACAGTGAAGTCCGATGACGTGGTCGGACGTGTGAAAACATATGAAGCCATCGTCAAA |
| | | GGCGACAATGTTCCAGAGCCTGGTGTTCCGGAATCATTCAAAGTATTGATCAAAGAGCT |
| | | TCAAAGCTTAGGTATGGACGTAAAAATCCTTTCAGGCGATGAAGAAGAAATAGAAATGA |
| | | GAGATCTAGAAGACGAGGAAGATGCGAAACAGGCTGACGGCCTTGCATTATCAGGTGAT |
| | | GAAGCGCCGGAAGAAACAGCATCTCCAGACGTTGAACGTGACGCAGTAACGAAAGAGTA |
| | | G |
| 15 | MS1835 | ATGTTTCAAGACATATCGCCAATTGAGGATTTCACTGGTAACCTCTCTCTGGAATTTAT |
| | | CGACTACAGCTTGGGCGAGCCTAAGTATCCGGTAGAAGAATCAAAAGAGCGGGATGTGA |
| | | CCTATTCAGCTCCGCTGCGGGTTAAAGTCCGCTTAATCAACAAAGAAACCGGCGAAGTA |
| | | AAAGATCAGGATGTCTTCATGGGCGATTTCCCTATTATGACAGACACTGGAACCTTCAT |
| | | TATCAACGGTGCAGAACGGGTCATCGTATCTCAGCTCGTTCGTTCTCCAAGTGTATATT |
| | | TTAGTGGTAAAGTAGACAAAAACGGTAAGAAAGGTTTTACCGCGACTGTCATTCCAAAC |
| | | CGTGGCGCATGGTTAGAATACGAGACTGATGCGAAAGATGTTGTTTACGTACGCATCGA |
| | | TCGCACACGTAAGTTGCCGGTTACGGTTCTTTTGCGTGCTCTTGGCTTCGGATCTGACC |
| | | AAGAGATCATTGATCTCATCGGTGAAAACGAGTATCTGCGCAATACGCTTGATAAAGAT |
| | | AATACGGAAAACACCGATAAAGCGCTTCTTGAAATCTACGAGCGTCTTCGTCCAGGGGA |
| | | GCCGCCTACCGTAGAAAACGCAAAAAGCCTGCTTGATTCAAGGTTCTTTGATCCGAAAA |
| | | GATATGACCTTGCGAGTGTAGGACGTTATAAAATTAATAAAAAGCTTCACATCAAAAAC |
| | | CGACTTTTTAATCAGCGGCTTGCTGAAACGCTAGTCGACCCTGAAACAGGCGAAATCCT |
| | | TGCCGAAAAAGGAGCGATTTTAGACAGAAGAACGCTTGATAAAGTTCTGCCGTACCTTG |
| | | AAAACGGAATCGGTTTTAAAAAGCTTTATCCGAACGGCGGAGTTGTCGAAGACGAAGTA |
| | | ACGCTTCAGTCTATCAAAATCTATGCTCCGACAGACCAAGAAGGGGAGCAGACAATCAA |
| | | TGTGATTGGAAATGCTTATATCGAAGAAGGCGTTAAGAATATTACACCTTCTGATATTA |
| | | TCGCTTCCATCAGCTATTTCTTTAACCTGCTTCACGGAGTGGGCGATACCGACGATATC |
| | | GACCATCTAGGAAACCGCCGTCTCCGTTCAGTGGGAGAGCTTCTGCAAAACCAATTCCG |
| | | TATTGGTTTAAGCAGAATGGAGCGCGTTGTTCGTGAAAGAATGTCTATTCAAGATACAA |
| | | ACACGATCACGCCGCAGCAGCTGATCAATATTCGCCCTGTCATCGCATCAATCAAAGAG |
| | | TTTTTCGGAAGCTCGCAGCTTTCTCAGTTTATGGATCAGACGAATCCGCTTGCTGAGCT |
| | | GACGCATAAGCGCCGTCTGTCAGCGCTCGGACCGGGCGGTTTGACTCGTGAGCGCGCCG |
| | | GAATGGAAGTCCGTGACGTTCACTATTCACACTACGGCCGGATGTGTCCGATTGAAACC |
| | | CCTGAGGGTCCAAACATCGGCTTGATCAACTCGCTTTCTTCATTCGCGAAAGTGAACCG |
| | | TTTCGGCTTCATCGAAACGCCGTATCGCCGCGTCGATCCTGAAACTGGAAAAGTAACGC |
| | | CGAGAATCGATTACTTGACAGCTGATGAAGAAGACAACTATGTCGTTGCCCAGGCAAAC |
| | | GCACGCCTGAATGATGACGGTTCTTTTGTGGATGACAGCATCGTCGCCCGTTTCAGAGG |
| | | GGAGAACACCGTTGTTCCGAAAGACCGCGTTGACTATATGGACGTTTCGCCTAAACAGG |
| | | TTGTCTCTGCCGCGACTGCATGTATTCCTTTCTTGGAAAACGATGACTCAAACCGCGCC |
| | | CTTATGGGAGCGAACATGCAACGTCAGGCCGTACCTCTTATGCAGCCTGAATCGCCGAT |
| | | CGTCGGAACCGGGATGGAATATGTATCTGCGAAAGACTCCGGTGCCGCTGTTATTTGCC |
| | | GCCACCCTGGAATCGTTGAACGGGTGGAAGCGAAGAACATCTGGGTGCGCCGCTATGAA |
| | | GAAGTCGACGGCCAGAAAGTCAAAGGGAACCTTGATAAATACAGCCTGCTGAAGTTTGT |
| | | CCGTTCCAACCAGGGAACTTGCTACAACCAGCGTCCGATCGTAAGCGTCGGTGATGAGG |
| | | TTGAAAAAGGTGAAATTTTAGCTGACGGTCCGTCCATGGAAAAAGGTGAGCTTGCCCTT |
| | | GGACGCAACGTCATGGTCGGCTTTATGACATGGGATGGCTACAACTATGAGGATGCCAT |
| | | CATCATGAGCGAACGCCTTGTAAAAGACGACGTATACACGTCTATTCATATTGAAGAAT |
| | | ACGAATCAGAGGCCCGGGATACAAAACTCGGACCTGAAGAAATCACTCGCGATATTCCG |
| | | AACGTCGGTGAGGACGCTCTTCGCAATCTCGATGAACGCGGAATTATCCGTGTCGGTGC |
| | | TGAAGTAAAAGACGGAGATCTTCTTGTTGGTAAAGTAACCCCTAAAGGTGTTACAGAGC |
| | | TTACGGCGGAAGAACGCCTGCTTCATGCCATCTTCGGGGAAAAAGCGCGTGAAGTGCGT |
| | | GATACGTCGCTGCGTGTACCTCACGGAGGCGGCGGTATCATCCTTGATGTAAAAGTGTT |
| | | CAACCGCGAAGACGGAGACGAACTGCCTCCGGGCGTTAACCAGCTCGTCCGCGTCTACA |
| | | TCGTTCAGAAGCGTAAAATTTCTGAAGGGGACAAAATGGCCGGACGCCACGGTAACAAA |
| | | GGTGTTATTTCGAAAATTCTTCCGGAGGAAGATATGCCGTATCTGCCTGACGGAACGCC |
| | | AATTGACATCATGTTAAACCCGCTGGGCGTACCATCGCGTATGAACATCGGGCAGGTGT |
| | | TGGAGCTGCACCTTGGTATGGCTGCACGCCGCCTCGGTCTGCATGTCGCGTCACCTGTA |
| | | TTTGACGGTGCCCGCGAAGAAGATGTGTGGGAAACCCTTGAAGAAGCCGGCATGTCAAG |
| | | AGACGCAAAAACCGTCCTTTACGACGGCCGAACTGGGGAGCCGTTTGACAACCGGGTTT |
| | | CTGTCGGCATCATGTACATGATCAAACTGGCACACATGGTTGACGACAAATTGCACGCA |
| | | CGTTCAACCGGTCCTTACTCACTCGTTACCCAGCAGCCTCTCGGAGGTAAAGCGCAGTT |
| | | CGGCGGACAGCGTTTTGGAGAGATGGAAGTTTGGGCGCTTGAAGCTTATGGTGCAGCAT |
| | | ATACGCTACAAGAGATCCTGACTGTTAAATCGGATGATGTCGTAGGCCGTGTGAAAACA |
| | | TATGAAGCCATCGTAAAAGGCGACAATGTTCCAGAACCTGGTGTTCCGGAATCGTTCAA |
| | | AGTATTGATCAAAGAGCTTCAAAGCTTAGGTATGGACGTCAAAATTCTATCAAGCGACG |
| | | AAGAAGAAATCGAAATGAGAGACTTGGAAGACGACGAAGACGCGAAACAAAACGAAGGG |
| | | CTTTCTCTGCCGAATGATGAAGAGTCCGAAGAGTTAGTTTCTGCTGACGCAGAACGCGA |
| | | TGTCGTTACAAAAGAATAA |

Incubation of a microbial consortium in the bioreactor system under conditions of a selective pressure source (e.g., providing only insoluble phosphate source) may enrich a microbial community with plant growth promotion properties (e.g., phosphate solubilizing microbes) and/or increase phosphate solubilization activity of a microbial community with or without a measurable increase in the population of phosphate solubilizers in the microbial community. Incubation of a phosphate-solubilizing target microbe and/or the phosphate-solubilizing microbes of the microbial consortium may generate metabolites with desired plant growth promotion properties (e.g., phosphate-solubilizing metabolites). The target phosphate-solubilizing microbe, phosphate-solubilizing microbes of the microbial consortium, phosphate-solubilizing metabolites, or any combination thereof may be phosphate-solubilizers in the working fluid of the system and/or in the output product (e.g., base product) of the bioreactor system.

In some embodiments, the absolute population of the microbial inoculum of the bioreactor system may not decrease by more than about 0.000001%, 0.00001%, 0.0001%, 0.001%, 0.01%, 0.1%, 0.5%, 1%, 2%, 3%, 4%, 5%, 6%, 7%, 8%, 9%, 10%, 15%, 20%, or 25% after incubation. In some embodiments, the absolute population of the microbial inoculum of the bioreactor system may not decrease by more than about 0.000001%, 0.00001%, 0.0001%, 0.001%, 0.01%, 0.1%, 0.5%, 1%, 2%, 3%, 4%, 5%, 6%, 7%, 8%, 9%, 10%, 15%, 20%, or 25% after incubation for a retention period of a bioreactor system (e.g., 1 day, 2 days, 3 days, 4 days, 5 days, 6 days, 1 week, 2 weeks, 3 weeks, 4 weeks, 2 months, 3 months, 4 months, 5 months, 6 months, 1 year, 2 years, 3 years, 4 years, or 5 years). In some embodiments, the absolute population of the microbial inoculum of the bioreactor system may not decrease by more than about 0.000001%, 0.00001%, 0.0001%, 0.001%, 0.01%, 0.1%, 0.5%, 1%, 2%, 3%, 4%, 5%, 6%, 7%, 8%, 9%, 10%, 15%, 20%, or 25% after incubation without inoculation of additional microbe.

In some embodiments, a proportion of a concentration of phosphate solubilizers relative to a total bacterial population count may increase across containers of a fluidly connected bioreactor system or may stay the same over a given period of time. In some embodiments, phosphate solubilizing activity by microbes present in a bioreactor system (e.g., one or more containers of a bioreactor system) may be enhanced without a measurable increase in the proportion of a concentration of phosphate solubilizers relative to a total bacterial population.

Phosphate solubilization can result in the production of phosphate ions (PO₄³⁻) as an insoluble source of phosphate is solubilized by microbes described herein. A measure of phosphate solubilization and capacity of microbes to solubilize phosphate can be an amount of phosphate ions (PO₄³⁻) made soluble. In some embodiments, the phosphate solubilization capacity (e.g., as PO₄³⁻ mg/mL made soluble) of a working solution may increase in a bioreactor system provided herein. In some embodiments, the phosphate solubilization capacity (e.g., as PO₄³⁻ mg/L made soluble) of a working solution may be at least about 1.5× higher, at least about 2× higher, at least about 3× higher, at least about 4× higher, at least about 5× higher, at least about 10× higher, at least about 20× higher, at least about 30× higher, at least about 40× higher, at least about 50× higher, at least about 60× higher, at least about 70× higher, at least about 80× higher, at least about 90× higher, or at least about 100× higher, or greater than about 100× higher in a second container, third container, or fourth container, compared to that in a first container of the bioreactor system or compared to a similar system that lacks one or more of the features of embodiments described herein such as, for example, a population of a phosphate-solubilizing target microbe, or insoluble phosphate as the primary or only phosphate source. In some embodiments, the phosphate solubilization capacity (e.g., as PO₄³⁻ g/mL made soluble) of a working solution may be at most about 100× higher, at most about 90× higher, at most about 80× higher, at most about 70× higher, at most about 60× higher, at most about 50× higher, at most about 40× higher, at most about 30× higher, at most about 20× higher, at most about 10× higher, at most about 5× higher, at most about 4× higher, at most about 4× higher, at most about 2× higher, or at most about 1.5× higher, or less than about 1.5× higher in a second container, third container, or fourth container, compared to that in a first container of the bioreactor system or compared to a similar system that lacks one or more of the features of embodiments described herein such as, for example, a population of a phosphate-solubilizing target microbe, or insoluble phosphate as the primary or only phosphate source.

In some embodiments, a working solution may be obtained from a reactor of a bioreactor system and tested for phosphate solubilizing capacity (e.g., quantified as PO₄³⁻ g/L made soluble) according to assays described herein. In some embodiments, a working solution of a bioreactor system described herein may have a measure of PO₄³⁻ made soluble of at least about 50 mg/L, at least about 100 mg/L, at least about 150 mg/L, at least about 200 mg/L, at least about 250 mg/L, at least about 300 mg/L, at least about 350 mg/L, at least about 400 mg/L, at least about 450 mg/L, at least about 500 mg/L, at least about 600 mg/L, at least about 700 mg/L, at least about 800 mg/L, or greater than about 800 mg/L. In some embodiments, a working solution of a bioreactor system described herein may have a measure of PO₄³⁻ made soluble of at most about 800 mg/L, at most about 700 mg/L, at most about 600 mg/L, at most about 500 mg/L, at most about 450 mg/L, at most about 400 mg/L, at most about 350 mg/L, at most about 300 mg/L, at most about 250 mg/L, at most about 200 mg/L, at most about 150 mg/L, at most about 100 mg/L, at most about 50 mg/L, or less than about 50 mg/L. In some embodiments, a working solution of a bioreactor system described herein may have a measure of PO₄³⁻ made soluble from about 20 mg/L to about 750 mg/L. In some embodiments, a working solution of a bioreactor system described herein may have a measure of PO₄³⁻ made soluble from about 20 mg/L to about 50 mg/L, about 20 mg/L to about 100 mg/L, about 20 mg/L to about 150 mg/L, about 20 mg/L to about 200 mg/L, about 20 mg/L to about 250 mg/L, about 20 mg/L to about 300 mg/L, about 20 mg/L to about 350 mg/L, about 20 mg/L to about 400 mg/L, about 20 mg/L to about 450 mg/L, about 20 mg/L to about 500 mg/L, about 20 mg/L to about 750 mg/L, about 50 mg/L to about 100 mg/L, about 50 mg/L to about 150 mg/L, about 50 mg/L to about 200 mg/L, about 50 mg/L to about 250 mg/L, about 50 mg/L to about 300 mg/L, about 50 mg/L to about 350 mg/L, about 50 mg/L to about 400 mg/L, about 50 mg/L to about 450 mg/L, about 50 mg/L to about 500 mg/L, about 50 mg/L to about 750 mg/L, about 100 mg/L to about 150 mg/L, about 100 mg/L to about 200 mg/L, about 100 mg/L to about 250 mg/L, about 100 mg/L to about 300 mg/L, about 100 mg/L to about 350 mg/L, about 100 mg/L to about 400 mg/L, about 100 mg/L to about 450 mg/L, about 100 mg/L to about 500 mg/L, about 100 mg/L to about 750 mg/L, about 150 mg/L to about 200 mg/L, about 150 mg/L to about 250 mg/L, about 150 mg/L to about 300 mg/L, about 150 mg/L to about 350 mg/L, about 150 mg/L to about 400 mg/L, about 150 mg/L to about 450 mg/L, about 150 mg/L to about 500 mg/L, about 150 mg/L to about 750 mg/L, about 200 mg/L to about 250 mg/L, about 200 mg/L to about 300 mg/L, about 200 mg/L to about 350 mg/L, about 200 mg/L to about 400 mg/L, about 200 mg/L to about 450 mg/L, about 200 mg/L to about 500 mg/L, about 200 mg/L to about 750 mg/L, about 250 mg/L to about 300 mg/L, about 250 mg/L to about 350 mg/L, about 250 mg/L to about 400 mg/L, about 250 mg/L to about 450 mg/L, about 250 mg/L to about 500 mg/L, about 250 mg/L to about 750 mg/L, about 300 mg/L to about 350 mg/L, about 300 mg/L to about 400 mg/L, about 300 mg/L to about 450 mg/L, about 300 mg/L to about 500 mg/L, about 300 mg/L to about 750 mg/L, about 350 mg/L to about 400 mg/L, about 350 mg/L to about 450 mg/L, about 350 mg/L to about 500 mg/L, about 350 mg/L to about 750 mg/L, about 400 mg/L to about 450 mg/L, about 400 mg/L to about 500 mg/L, about 400 mg/L to about 750 mg/L, about 450 mg/L to about 500 mg/L, about 450 mg/L to about 750 mg/L, or about 500 mg/L to about 750 mg/L.

Population of the microbe may be measured using methods including but not limited to spectrophotometers, cell counting, measures of turbidity, hemocytometers, electronic enumeration, measures of cell mass, and measures of cell activity.

Phosphate-solubilizing microbes may be identified using methods using agar plates or liquid testing solutions. Microbes may be tested on agar plates with insoluble phosphate and the measurement of phosphate solubilization capacity (e.g., amount of phosphate made soluble) may classify a microbe as capable of phosphate solubilization. Microbes may be tested in liquid solution containing insoluble phosphate to measure phosphate solubilization capacity. Collective phosphate solubilization activity of a community of microbes within a working fluid, product stream, or other sample from the system may be measured using assays described herein to determine whether desired stimulation of phosphate solubilizing activity is being achieved. Phosphate solubilization capacity may be measured at different time points and monitored to observe the functioning of the system.

The addition of the target isolate (e.g., the inoculum of the microbial strain/microbe) can have an added benefit in improving target functionality of working solution in a reactor of the bioreactor system and/or in the output base product of the bioreactor system. In some embodiments, addition of the inoculum of the microbial strain can improve the phosphate solubilization capacity of working fluids and base product of a bioreactor system compared to working fluids and base product of a bioreactor system without an inoculum of the microbial strain.

In some embodiments, a working solution and/or base product of a bioreactor system inoculated with a target isolate may have an at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, or greater than 80% increase in PO₄³⁻ concentration in comparison with that of a working solution and/or base product of a bioreactor system not inoculated with a target isolate. In some embodiments, a working solution and/or base product of a bioreactor system inoculated with a target isolate may have an at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15% or less than about 15% increase in PO₄³⁻ concentration in comparison with that of a working solution and/or base product of a bioreactor system not inoculated with a target isolate.

In some embodiments, a working solution and/or base product of a bioreactor system inoculated with a target isolate may have from about 10% to about 80% increase in PO₄³⁻ solubilization activity in comparison with that of a working solution and/or base product of a bioreactor system not inoculated with a target isolate. In some embodiments, a working solution and/or base product of a bioreactor system inoculated with a target isolate may have from about 10% to about 15%, about 10% to about 20%, about 10% to about 25%, about 10% to about 30%, about 10% to about 35%, about 10% to about 40%, about 10% to about 45%, about 10% to about 50%, about 10% to about 60%, about 10% to about 70%, about 10% to about 80%, about 15% to about 20%, about 15% to about 25%, about 15% to about 30%, about 15% to about 35%, about 15% to about 40%, about 15% to about 45%, about 15% to about 50%, about 15% to about 60%, about 15% to about 70%, about 15% to about 80%, about 20% to about 25%, about 20% to about 30%, about 20% to about 35%, about 20% to about 40%, about 20% to about 45%, about 20% to about 50%, about 20% to about 60%, about 20% to about 70%, about 20% to about 80%, about 25% to about 30%, about 25% to about 35%, about 25% to about 40%, about 25% to about 45%, about 25% to about 50%, about 25% to about 60%, about 25% to about 70%, about 25% to about 80%, about 30% to about 35%, about 30% to about 40%, about 30% to about 45%, about 30% to about 50%, about 30% to about 60%, about 30% to about 70%, about 30% to about 80%, about 35% to about 40%, about 35% to about 45%, about 35% to about 50%, about 35% to about 60%, about 35% to about 70%, about 35% to about 80%, about 40% to about 45%, about 40% to about 50%, about 40% to about 60%, about 40% to about 70%, about 40% to about 80%, about 45% to about 50%, about 45% to about 60%, about 45% to about 70%, about 45% to about 80%, about 50% to about 60%, about 50% to about 70%, about 50% to about 80%, about 60% to about 70%, about 60% to about 80%, or about 70% to about 80% increase in PO₄³⁻ solubilization activity in comparison with that of a working solution and/or base product of a bioreactor system not inoculated with a target isolate.

A bioreactor system may be inoculated with a microbial strain at the start of a bioreactor system, which may allow for the microbial strain to flow through the system and working fluids of the reactors. Without wishing to be bound by theory, a bioreactor system inoculated with a phosphate solubilizing microbial strain (for example, one of the strains described herein) may increase a phosphate solubilization capacity of a working fluid and/or a base product compared to a working fluid and/or base product of an otherwise identical bioreactor system with no inoculated phosphate solubilizing microbial strain. Without wishing to be bound by theory, a bioreactor system inoculated with a phosphate solubilizing microbial strain may increase a phosphate solubilization capacity of a working fluid and/or a base product compared to a working fluid and/or base product of an otherwise identical bioreactor system with the phosphate solubilizing microbial strain added (e.g., spiked) at the end of the system. Without wishing to be bound by theory, a bioreactor system inoculated with a phosphate solubilizing microbial strain may increase a phosphate solubilization capacity of a base product compared to a base product of an otherwise identical bioreactor system with the phosphate solubilizing microbial strain added (e.g., spiked) at the end of the system. Incubation of the phosphate solubilizing microbial strain in the bioreactor system may enrich the working fluid with phosphate-solubilizing microbes, stimulate phosphate-solubilizing activity of microbes in the system with or without an increase in the population of phosphate-solubilizing microbes in the system, and/or generate phosphate-solubilizing metabolites.

### C. Nitrogen Use Efficiency Microbial Isolates

In some embodiments, other desirable microbes may have nitrogen use efficiency-promoting properties (e.g., enhancing nitrogen-fixing activity, improving recruitment of plant associated nitrogen fixers already present in the soil to the roots of plants, increasing soil organic nitrogen and mineralization and uptake of organic nitrogen stimulated by microbes and/or microbial metabolites, or any combination thereof). The other desirable microbes may be of the bacterial genera: Kosakonia, Klebsiella, Rahnella, Kluyvera, Enterobacter, Achromobacter, Microbacterium, Gluconobacter, Methylobacterium, Pseudomonas, Pantoea, Azospirillum, Azocarus, Herbaspirillum, Burkholderia, Cyanobacteria, Bacillus, Paenibacillus, or any combination thereof. The other desirable microbes may be of the bacterial species Kosakonia sacchari, Klebsiella variicola, Rahnella aquatilis, Kluyvera intermedia, Kosakonia pseusosacchari, Enterobacter spp., Achromobacter marplatensis, Azopirillum lipoferum, Microbacterium murale, Gluconobacter diazotrophicus, Methylobacterium symbioticum, Paenibacillus borealis, Bacillus megaterium (Priestia megaterium), Paenibacillus sonchi, or any combination thereof.

In some embodiments, operating a bioreactor system disclosed herein provides a synergistic effect in the production of a biostimulant composition. In some embodiments, a bioreactor system that comprises an established and/or stable population of a nitrogen use efficiency (NUE)-promoting microbial strain (e.g., one or more of the NUE-promoting strains disclosed herein and/or related strains) produces a biostimulant having a greater nitrogen use efficiency-promoting effect than could be obtained from an otherwise similar bioreactor system that does not have the established population of the NUE-promoting strain. The nitrogen use efficiency-promoting effect of the biostimulant produced according to methods disclosed herein may be greater than a biostimulant produced in an otherwise similar system lacking the established population of the nitrogen use efficiency-promoting strain, even if the latter biostimulant has one or more of the NUE-promoting strains added to it after it is produced. For example, a biostimulant composition produced according to the methods disclosed herein may have greater nitrogen-fixing activity effects compared to a biostimulant composition produced by an otherwise similar system lacking the established population of the nitrogen use efficiency-promoting strain. For example, a biostimulant composition produced according to the methods disclosed herein may have greater recruitment capacity of plant associated nitrogen fixers present in the soil to the roots of plants compared to that of a biostimulant composition produced by an otherwise similar system lacking the established population of the nitrogen use efficiency-promoting strain. For example, a biostimulant composition produced according to the methods disclosed herein may have greater capacity to increase soil organic nitrogen and mineralization and uptake of organic nitrogen compared to that of a biostimulant composition produced by an otherwise similar system lacking the established population of the nitrogen use efficiency-promoting strain. Without being bound by theory, the presence of the target NUE-promoting strain population in the bioreactor system may promote production of NUE-promoting digestion products in the bioreactor system, which are present in the product.

In some embodiments, the microbial strain is from a Bacillus genus. In some embodiments, the bacterial strain is from a Bacillus megaterium species. In some embodiments, the bacterial strain is from a bacterial species other than Bacillus megaterium. In some embodiments, the Bacillus megaterium strain is the strain deposited under ATCC Accession No. PTA-127653, or an isolated clone thereof. In some embodiments, the 16S rRNA gene of the microbial strain comprises the nucleotide sequence of SEQ ID NO: 16. In some embodiments, the microbial strain comprises a 16S rRNA gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 16. In some embodiments, the gyrB gene of the microbial strain comprises the nucleotide sequence of SEQ ID NO: 20. In some embodiments, the microbial strain comprises a gyrB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 20. In some embodiments, the rpoB gene of the microbial strain comprises the nucleotide sequence of SEQ ID NO: 24. In some embodiments, the microbial strain comprises a rpoB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 24.

In some embodiments, the bacterial strain is from a Paenibacillus borealis species. In some embodiments, the bacterial strain is from a bacterial species other than Paenibacillus borealis. In some embodiments, the Paenibacillus borealis strain is the strain deposited under ATCC Accession No. PTA-127654, or an isolated clone thereof. In some embodiments, the 16S rRNA gene of the microbial strain comprises the nucleotide sequence of SEQ ID NO: 17. In some embodiments, the microbial strain comprises a 16S rRNA gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 17. In some embodiments, the gyrB gene of the microbial strain comprises the nucleotide sequence of SEQ ID NO: 21. In some embodiments, the microbial strain comprises a gyrB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 21. In some embodiments, the rpoB gene of the microbial strain comprises the nucleotide sequence of SEQ ID NO: 25. In some embodiments, the microbial strain comprises a rpoB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 25.

In some embodiments, the bacterial strain is from a Paenibacillus sonchi species. In some embodiments, the bacterial strain is from a bacterial species other than Paenibacillus sonchi. In some embodiments, the Paenibacillus sonchi strain is the strain deposited under ATCC Accession No. PTA-127655, or an isolated clone thereof. In some embodiments, the 16S rRNA gene of the microbial strain comprises the nucleotide sequence of SEQ ID NO: 18. In some embodiments, the microbial strain comprises a 16S rRNA gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 18.

In some embodiments, the gyrB gene of the microbial strain comprises the nucleotide sequence of SEQ ID NO: 22. In some embodiments, the microbial strain comprises a gyrB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 22. In some embodiments, the rpoB gene of the microbial strain comprises the nucleotide sequence of SEQ ID NO: 26. In some embodiments, the microbial strain comprises a rpoB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 26.

In some embodiments, the bacterial strain is from a Bacillus megaterium species. In some embodiments, the bacterial strain is from a bacterial species other than Bacillus megaterium. In some embodiments, the Bacillus megaterium strain is the strain deposited under ATCC Accession No. PTA-127652, or an isolated clone thereof. In some embodiments, the 16S rRNA gene of the microbial strain comprises the nucleotide sequence of SEQ ID NO: 19. In some embodiments, the microbial strain comprises a 16S rRNA gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 19. In some embodiments, the gyrB gene of the microbial strain comprises the nucleotide sequence of SEQ ID NO: 23. In some embodiments, the microbial strain comprises a gyrB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 23. In some embodiments, the rpoB gene of the microbial strain comprises the nucleotide sequence of SEQ ID NO: 27. In some embodiments, the microbial strain comprises a rpoB gene comprising a nucleotide sequence that exhibits at least 80%, at least 85%, at least 90%, at least 91%, at least 92%, at least 93%, at least 94%, at least 95%, at least 96%, at least 97%, at least 98%, at least 99%, at least 99.9%, or 100% sequence identity to a nucleotide sequence as set forth in SEQ ID NO: 27.

TABLE 7
Exemplary 16S rRNA Sequences of Bacterial Isolates

| SEQ ID | | |
|---|---|---|
| NO. | Bacterial Gene | Sequence |
| 16 | MS3900 | CATTTCTTCGGAGAGTTTGATCCTGGCTCAGGATGAACGCTGG |
| | | CGGCGTGCCTAATACATGCAAGTCGAGCGAACTGATTAGAAGC |
| | | TTGCTTCTATGACGTTAGCGGCGGACGGGTGAGTAACACGTGG |
| | | GCAACCTGCCTGTAAGACTGGGATAACTTCGGGAAACCGAAGC |
| | | TAATACCGGATAGGATCTTCTCCTTCATGGGAGATGATTGAAA |
| | | GATGGTTTCGGCTATCACTTACAGATGGGCCCGCGGTGCATTA |
| | | GCTAGTTGGTGAGGTAACGGCTCACCAAGGCAACGATGCATAG |
| | | CCGACCTGAGAGGGTGATCGGCCACACTGGGACTGAGACACGG |
| | | CCCAGACTCCTACGGGAGGCAGCAGTAGGGAATCTTCCGCAAT |
| | | GGACGAAAGTCTGACGGAGCAACGCCGCGTGAGTGATGAAGGC |
| | | TTTCGGGTCGTAAAACTCTGTTGTTAGGGAAGAACAAGTACAA |
| | | GAGTAACTGCTTGTACCTTGACGGTACCTAACCAGAAAGCCAC |
| | | GGCTAACTACGTGCCAGCAGCCGCGGTAATACGTAGGTGGCAA |
| | | GCGTTATCCGGAATTATTGGGCGTAAAGCGCGCGCAGGCGGTT |
| | | TCTTAAGTCTGATGTGAAAGCCCACGGCTCAACCGTGGAGGGT |
| | | CATTGGAAACTGGGGAACTTGAGTGCAGAAGAGAAAAGCGGAA |
| | | TTCCACGTGTAGCGGTGAAATGCGTAGAGATGTGGAGGAACAC |
| | | CAGTGGCGAAGGCGGCTTTTTGGTCTGTAACTGACGCTGAGGC |
| | | GCGAAAGCGTGGGGAGCAAACAGGATTAGATACCCTGGTAGTC |
| | | CACGCCGTAAACGATGAGTGCTAAGTGTTAGAGGGTTTCCGCC |
| | | CTTTAGTGCTGCAGCTAACGCATTAAGCACTCCGCCTGGGGAG |
| | | TACGGTCGCAAGACTGAAACTCAAAGGAATTGACGGGGGCCCG |
| | | CACAAGCGGTGGAGCATGTGGTTTAATTCGAAGCAACGCGAAG |
| | | AACCTTACCAGGTCTTGACATCCTCTGACAACTCTAGAGATAG |
| | | AGCGTTCCCCTTCGGGGGACAGAGTGACAGGTGGTGCATGGTT |
| | | GTCGTCAGCTCGTGTCGTGAGATGTTGGGTTAAGTCCCGCAAC |
| | | GAGCGCAACCCTTGATCTTAGTTGCCAGCATTTAGTTGGGCAC |
| | | TCTAAGGTGACTGCCGGTGACAAACCGGAGGAAGGTGGGGATG |
| | | ACGTCAAATCATCATGCCCCTTATGACCTGGGCTACACACGTG |
| | | CTACAATGGATGGTACAAAGGGCTGCAAGACCGCGAGGTCAAG |
| | | CCAATCCCATAAAACCATTCTCAGTTCGGATTGTAGGCTGCAA |
| | | CTCGCCTACATGAAGCTGGAATCGCTAGTAATCGCGGATCAGC |
| | | ATGCCGCGGTGAATACGTTCCCGGGCCTTGTACACACCGCCCG |
| | | TCACACCACGAGAGTTTGTAACACCCGAAGTCGGTGGAGTAAC |
| | | CGTAAGGAGCTAGCCGCCTAAGGTGGGACAGATGATTGGGGTG |
| | | AAGTCGTAACAAGGTAGCCGTATCGGAAGGTGCGGCTGGATCA |
| | | CCTCCTTTCTA |
| 17 | MS3907 | TTCAATACAAATTGGAGAGTTTGATCCTGGCTCAGGACGAACG |
| | | CTGGCGGCGTGCCTAATACATGCAAGTCGAGCGGAGTTATGAT |
| | | GGAGCTTGCTCCTGATTAACTTAGCGGCGGACGGGTGAGTAAC |
| | | ACGTAGGCAACCTGCCCTCAAGACTGGGATAACTACCGGAAAC |
| | | GGTAGCTAATACCGGATAATTTCTTTGTTCTCCTGAAGAGAGA |
| | | ATGAAAGGCGGAGCAATCTGCCACTTGAGGATGGGCCTGCGGC |
| | | GCATTAGCTAGTTGGTGAGGTAACGGCTCACCAAGGCGACGAT |
| | | GCGTAGCCGACCTGAGAGGGTGAACGGCCACACTGGGACTGAG |
| | | ACACGGCCCAGACTCCTACGGGAGGCAGCAGTAGGGAATCTTC |
| | | CGCAATGGGCGAAAGCCTGACGGAGCAACGCCGCGTGAGTGAT |
| | | GAAGGTTTTCGGATCGTAAAGCTCTGTTGCCAGGGAAGAACGT |
| | | CCGGTAGAGTAACTGCTACCGGAGTGACGGTACCTGAGAAGAA |
| | | AGCCCCGGCTAACTACGTGCCAGCAGCCGCGGTAATACGTAGG |
| | | GGGCAAGCGTTGTCCGGAATTATTGGGCGTAAAGCGCGCGCAG |
| | | GCGGCGATTTAAGTCTGGTGTTTAAACCTTGGGCTCAACCTGG |
| | | GGTCGCACTGGAAACTGGATCGCTTGAGTACAGAAGAGGAAAG |
| | | TGGAATTCCACGTGTAGCGGTGAAATGCGTAGATATGTGGAGG |
| | | AACACCAGTGGCGAAGGCGACTTTCTGGGCTGTAACTGACGCT |
| | | GAGGCGCGAAAGCGTGGGGAGCAAACAGGATTAGATACCCTGG |
| | | TAGTCCACGCCGTAAACGATGAGTGCTAGGTGTTAGGGGTTTC |
| | | GATACCCTTGGTGCCGAAGTTAACACAGTAAGCACTCCGCCTG |
| | | GGGAGTACGGTCGCAAGACTGAAACTCAAAGGAATTGACGGGG |
| | | ACCCGCACAAGCAGTGGAGTATGTGGTTTAATTCGAAGCAACG |
| | | CGAAGAACCTTACCAGGTCTTGACATCCCGATGAAAGCATTAG |
| | | AGATAGTGCCCCTCTTCGGAGCATCGGAGACAGGTGGTGCATG |
| | | GTTGTCGTCAGCTCGTGTCGTGAGATGTTGGGTTAAGTCCCGC |
| | | AACGAGCGCAACCCTTGACTTTAGTTGCCAGCAGGTTAAGCTG |
| | | GGCACTCTAGAGTGACTGCCGGTGACAAACCGGAGGAAGGTGG |
| | | GGATGACGTCAAATCATCATGCCCCTTATGACCTGGGCTACAC |
| | | ACGTACTACAATGGCCGGTACAACGGGAAGCGAAGCCGCGAGG |
| | | TGGAGCCAATCCCAGCAAAGCCGGTCTCAGTTCGGATTGCAGG |
| | | CTGCAACTCGCCTGCATGAAGTCGGAATTGCTAGTAATCGCGG |
| | | ATCAGCATGCCGCGGTGAATACGTTCCCGGGTCTTGTACACAC |
| | | CGCCCGTCACACCACGAGAGTTTACAACACCCGAAGTCGGTGG |
| | | GGTAACCCGCAAGGGAGCCAGCCGCCGAAGGTGGGGTAGATGA |
| | | TTGGGGTGAAGTCGTAACAAGGTAGCCGTATCGGAAGGTGCGG |
| | | CTGGATCACCTCCTTTCTA |
| 18 | MS4921 | TACCAATTGGAGAGTTTGATCCTGGCTCAGGACGAACGCTGGC |
| | | GGCGTGCCTAATACATGCAAGTCGAGCGGAGTTTATCCTTCGG |
| | | GGTAAGCTTAGCGGCGGACGGGTGAGTAACACGTAGGCAACCT |
| | | ACCCTCTAGACTGGGATAACTACCGGAAACGGTAGCTAATACC |
| | | GGATAATTCCTTGATCCACATGGGCTAAGGATGAAAGGCGGAG |
| | | CAATCTGCTGCTAGAGGATGGGCCTGCGGCGCATTAGCTAGTT |
| | | GGTGGGGTAACGGCCTACCAAGGCGACGATGCGTAGCCGACCT |
| | | GAGAGGGTGAACGGCCACACTGGGACTGAGACACGGCCCAGAC |
| | | TCCTACGGGAGGCAGCAGTAGGGAATCTTCCGCAATGGGCGAA |
| | | AGCCTGACGGAGCAACGCCGCGTGAGTGATGAAGGTTTTCGGA |
| | | TCGTAAAGCTCTGTTGCCAGGGAAGAACGTCCGGTAGAGTAAC |
| | | TGCTATCGGAGTGACGGTACCTGAGAAGAAAGCCCCGGCTAAC |
| | | TACGTGCCAGCAGCCGCGGTAATACGTAGGGGGCAAGCGTTGT |
| | | CCGGAATTATTGGGCGTAAAGCGCGCGCAGGCGGCTGCTTAAG |
| | | TCTGGTGTTTAAACCTTGGGCTCAACCTGGGGTCGCACTGGAA |
| | | ACTGGGCAGCTTGAGTACAGAAGAGGAAAGTGGAATTCCACGT |
| | | GTAGCGGTGAAATGCGTAGAGATGTGGAGGAACACCAGTGGCG |
| | | AAGGCGACTTTCTGGGCTGTAACTGACGCTGAGGCGCGAAAGC |
| | | GTGGGGAGCAAACAGGATTAGATACCCTGGTAGTCCACGCCGT |
| | | AAACGATGAGTGCTAGGTGTTAGGGGTTTCGATACCCTTGGTG |
| | | CCGAAGTTAACACAGTAAGCACTCCGCCTGGGGAGTACGGTCG |
| | | CAAGACTGAAACTCAAAGGAATTGACGGGGACCCGCACAAGCA |
| | | GTGGAGTATGTGGTTTAATTCGAAGCAACGCGAAGAACCTTAC |
| | | CAGGTCTTGACATCCAACTAACGAAGCAGAGATGCATCAGGTG |
| | | CCCTTCGGGGAAAGTTGAGACAGGTGGTGCATGGTTGTCGTCA |
| | | GCTCGTGTCGTGAGATGTTGGGTTAAGTCCCGCAACGAGCGCA |
| | | ACCCTTGACTTTAGTTGCCAGCAGGTGAAGCTGGGCACTCTAG |
| | | AGTGACTGCCGGTGACAAACCGGAGGAAGGTGGGGATGACGTC |
| | | AAATCATCATGCCCCTTATGACCTGGGCTACACACGTACTACA |
| | | ATGGCCGGTACAACGGGAAGCGAAGCCGCGAGGTGGAGCCAAT |
| | | CCCAGCAAAGCCGGTCTCAGTTCGGATTGCAGGCTGCAACTCG |
| | | CCTGCATGAAGTCGGAATTGCTAGTAATCGCGGATCAGCATGC |
| | | CGCGGTGAATACGTTCCCGGGTCTTGTACACACCGCCCGTCAC |
| | | ACCACGAGAGTTTACAACACCCGAAGTCGGTGGGGTAACCCGC |
| | | AAGGGGGCCAGCCGCCGAAGGTGGGGTAGATGATTGGGGTGAA |
| | | GTCGTAACAAGGTAGCCGTATCGGAAGGTGCGGCTGGATCACC |
| | | TCCTTTCTA |
| 19 | MS2748 | CATTTCTTCGGAGAGTTTGATCCTGGCTCAGGATGAACGCTGG |
| | | CGGCGTGCCTAATACATGCAAGTCGAGCGAACTGATTAGAAGC |
| | | TTGCTTCTATGACGTTAGCGGCGGACGGGTGAGTAACACGTGG |
| | | GCAACCTGCCTGTAAGACTGGGATAACTTCGGGAAACCGAAGC |
| | | TAATACCGGATAGGATCTTCTCCTTCATGGGAGATGATTGAAA |
| | | GATGGTTTCGGCTATCACTTACAGATGGGCCCGCGGTGCATTA |
| | | GCTAGTTGGTGAGGTAACGGCTCACCAAGGCAACGATGCATAG |
| | | CCGACCTGAGAGGGTGATCGGCCACACTGGGACTGAGACACGG |
| | | CCCAGACTCCTACGGGAGGCAGCAGTAGGGAATCTTCCGCAAT |
| | | GGACGAAAGTCTGACGGAGCAACGCCGCGTGAGTGATGAAGGC |
| | | TTTCGGGTCGTAAAACTCTGTTGTTAGGGAAGAACAAGTACGA |
| | | GAGTAACTGCTCGTACCTTGACGGTACCTAACCAGAAAGCCAC |
| | | GGCTAACTACGTGCCAGCAGCCGCGGTAATACGTAGGTGGCAA |
| | | GCGTTATCCGGAATTATTGGGCGTAAAGCGCGCGCAGGCGGTT |
| | | TCTTAAGTCTGATGTGAAAGCCCACGGCTCAACCGTGGAGGGT |
| | | CATTGGAAACTGGGGAACTTGAGTGCAGAAGAGAAAAGCGGAA |
| | | TTCCACGTGTAGCGGTGAAATGCGTAGAGATGTGGAGGAACAC |
| | | CAGTGGCGAAGGCGGCTTTTTGGTCTGTAACTGACGCTGAGGC |
| | | GCGAAAGCGTGGGGAGCAAACAGGATTAGATACCCTGGTAGTC |
| | | CACGCCGTAAACGATGAGTGCTAAGTGTTAGAGGGTTTCCGCC |
| | | CTTTAGTGCTGCAGCTAACGCATTAAGCACTCCGCCTGGGGAG |
| | | TACGGTCGCAAGACTGAAACTCAAAGGAATTGACGGGGGCCCG |
| | | CACAAGCGGTGGAGCATGTGGTTTAATTCGAAGCAACGCGAAG |
| | | AACCTTACCAGGTCTTGACATCCTCTGACAACTCTAGAGATAG |
| | | AGCGTTCCCCTTCGGGGGACAGAGTGACAGGTGGTGCATGGTT |
| | | GTCGTCAGCTCGTGTCGTGAGATGTTGGGTTAAGTCCCGCAAC |
| | | GAGCGCAACCCTTGATCTTAGTTGCCAGCATTAAGTTGGGCAC |
| | | TCTAAGGTGACTGCCGGTGACAAACCGGAGGAAGGTGGGGATG |
| | | ACGTCAAATCATCATGCCCCTTATGACCTGGGCTACACACGTG |
| | | CTACAATGGATGGTACAAAGGGCTGCAAGACCGCGAGGTCAAG |
| | | CCAATCCCATAAAACCATTCTCAGTTCGGATTGTAGGCTGCAA |
| | | CTCGCCTACATGAAGCTGGAATCGCTAGTAATCGCGGATCAGC |
| | | ATGCCGCGGTGAATACGTTCCCGGGCCTTGTACACACCGCCCG |
| | | TCACACCACGAGAGTTTGTAACACCCGAAGTCGGTGGAGTAAC |
| | | CGTAAGGAGCTAGCCGCCTAAGGTGGGACAGATGATTGGGGTG |
| | | AAGTCGTAACAAGGTAGCCGTATCGGAAGGTGCGGCTGGATCA |
| | | CCTCCTTTCTA |

TABLE 8
Exemplary gyrB Gene Sequences of Bacterial Isolates

| SEQ ID | | |
|---|---|---|
| NO. | Bacterial Gene | Sequence |
| 20 | MS3900 | TTGACGATGGAACAAAAAGAAGTACAAGCATATGAAGCTGATC |
| | | AGATACAAGTATTAGAAGGATTAGAAGCTGTTCGTAAACGTCC |
| | | GGGGATGTATATTGGATCGACGAGCGCAAAGGGTCTACATCAT |
| | | CTTGTATGGGAAATTGTAGATAATAGTATTGATGAAGCGCTGG |
| | | CCGGCTATTGCGATGAAATTAATGTTATTATCGAAAAGGATAA |
| | | TAGTATTACAGTCAAAGATAACGGTCGTGGAATTCCGGTTGGT |
| | | ATTCAAGAAAAAATGGGCAGACCTGCCGTTGAAGTTATCTTAA |
| | | CGGTTCTTCATGCCGGAGGTAAATTTGGCGGCGGCGGCTATAA |
| | | AGTATCTGGTGGATTACACGGTGTAGGTGCCTCAGTTGTTAAT |
| | | GCACTTTCTACCTCTTTGGAAGTGCACGTACATCGTGACGGTA |
| | | AAGTTCATTATCAAAAATATGAACGAGGTGTACCGGCTGCTGA |
| | | CTTAAAAGTAGTTGGAGAAACAGATAAAACAGGTACTGTTATT |
| | | CAATTCCGTCCAGACGGTGAAATTTTTACAGAAACGCTTGAAT |
| | | ACGATTTTGATACGTTAGCTAATCGTCTGCGTGAGTTAGCTTT |
| | | CTTAAATCGTGGCATTAAAATTACGATTGAAGACAAACGTGAA |
| | | GAAGATAAAAGACGTGAGTATCACTATGAAGGCGGAATTAAGT |
| | | CTTACGTTGAACACTTAAACCGTTCGAAAGAAGTGATTCACGA |
| | | AGAGCCAATCTATATTGAAGGTAATCGAGACAACATTTCTGTA |
| | | GAAATTGCTATTCAATATAACGATAGCTATACAAGTAATTTAT |
| | | ATTCTTTTGCAAACAATATTCACACATATGAAGGTGGAACGCA |
| | | CGAAGCAGGATTTAAAACAGCGTTAACGCGTGTAATTAACGAC |
| | | TATGCACGTAAAAACAGCGTATTTAAAGACAGTGACGCCAATC |
| | | TAACAGGTGAAGATGTTCGTGAAGGAATTACAGCTATCATCTC |
| | | TATTAAGCACCCAGATCCGCAGTTCGAAGGACAAACAAAAACA |
| | | AAGCTGGGAAATAGTGAAGCAAGAACAATTACTGACTCTGTGT |
| | | TTGCAGAACACTTAGAAACTTACTTGCTAGAGAACCCTATTGT |
| | | GGCGAAAAAGGTAATTGAAAAAGGTTTAATGGCTGCAAGAGCA |
| | | AGAATGGCAGCTAAAAAAGCTCGTGAGCTTACACGCCGTAAAA |
| | | GCGCGCTTGAAATTTCAAACTTACCGGGTAAATTAGCAGATTG |
| | | TTCATCAAAAGATCCTTCTATTAGCGAACTCTATGTAGTAGAG |
| | | GGTGACTCTGCCGGAGGTTCAGCTAAGCAGGGAAGAAGCCGTC |
| | | ATTTCCAAGCTATTTTGCCTTTACGTGGTAAAATTATCAACGT |
| | | AGAGAAAGCGCGTTTAGATAAAATTTTATCTAATAACGAAATT |
| | | CGTACAATCATTACCGCTCTAGGAACGGGTATTGGTGACGATT |
| | | TTGATATTTCGAAAGCCCGCTACCATAAAATTGTGATTATGAC |
| | | AGATGCAGACGTAGACGGTGCGCATATTCGTACGCTTCTTCTA |
| | | ACGTTCTTCTATCGCTATATGAGACAGATTATTGAGCACGGAT |
| | | ATGTGTACATTGCTCAGCCGCCTCTTTACAAAGTTTCACAAGG |
| | | TAAAAAAGTGGAGTACGCGTACAACGATCGTCAATTAGAAGAG |
| | | GTATTAGCTTCTTTCCCTGAAGGCGCAAAACCAAACCTTCAGC |
| | | GTTACAAAGGTTTAGGAGAGATGAATCCTGAACAATTATGGGA |
| | | AACAACAATGGATCCAGAGTTCCGTACCCTTCTTCAGGTGAAC |
| | | TTGCAAGATGCAATTGAAGCTGATGAGACATTTGAAATTTTAA |
| | | TGGGCGACAAAGTAGAACCACGCCGTAATTTCATTGAAGAAAA |
| | | TGCTCAGTACGTAAAAAATCTTGATATTTAA |
| 21 | MS3907 | ATGTCAATGAATCAACCGACATATGATGAGAGCCAGATTCAGG |
| | | TACTGGAAGGGCTGGAAGCGGTTCGGAAACGTCCCGGCATGTA |
| | | CATTGGCTCCACTAGCGCCAAAGGTCTGCATCATTTGGTCTGG |
| | | GAGGTCGTCGATAATAGTATCGATGAGGCGCTGGCAGGTTTTT |
| | | GCGACCGGATTCAAGTGATGATTCATGAAGATAACAGCGTGAC |
| | | TGTTATCGATAACGGACGGGGTATTCCTGTCGGAGAGAACGAG |
| | | AAGCTGAAGAAATCCACGCTTGAAGTTGTAATGACTGTCCTGC |
| | | ATGCAGGCGGTAAATTCGGCGGCGGGGGATATAAGGTTTCCGG |
| | | CGGTCTGCACGGTGTGGGGATCTCTGTTGTGAATGCATTGTCC |
| | | GAGAAGGTAGTTGTAAAGGTTAAACGTGACGGACACGTCTACC |
| | | AGCAGGAATACAGACGGGGTGCGCCGCAGTATGATATCAGGGT |
| | | AATCGGGGATACCGATGAGACTGGAACCACCACCACCTTCCAT |
| | | CCGGATCCTGAGATTTTTACCGAAACGACTATTTTTGAATATT |
| | | CAACTCTGCTTACCCGTATCCGCGAGCTCGCCTTCTTGAATAA |
| | | GGGTATCGAGCTGTCCCTGCTGGATGAACGGACCGGAGTCTCC |
| | | AATACATTTAAGTATGATGGCGGTATTGTGGAGTATGTGAAGT |
| | | ACCTTAACGAGAAAAAAGAAGCCCTGCACGAGGACCCGATCTA |
| | | CGTGGAAGGCTCACGGGATATGATCGCGGTTGAAGTGGCTCTC |
| | | CAATATAACGATTCATACTCAGAGAACATCTATTCCTTTGCCA |
| | | ACAATATCAACACTCATGAAGGCGGTACGCATGAATCCGGCTT |
| | | CAAGAGCGCCTTGACACGTATTATCAATGATTATGCCCGTAAG |
| | | GCTGGCGTTATCAAGGACAGCAGCCAGAATCTGACCGGAGATG |
| | | ATGTACGTGAAGGCTTGACGGCGATTATTTCCGTCAAAATTCC |
| | | CGAACCGCAGTTCGAAGGCCAGACCAAGACCAAGCTTGGGAAC |
| | | AGCGAAGTCCGCGGGATTGTGGAGTCCCTGTTCGGTGAGAAGC |
| | | TGCAGGAATTCCTGGAAGAGAACCCTGCTGTGTCCCGCCGCGT |
| | | GCTGGAGAAGTCGCTGCAGGCTTCACGTGCACGTGAAGCCGCG |
| | | CGCAAAGCCCGTGAACTGACCCGCCGCAAGAGTGCGCTTGAAG |
| | | TCAGTGCCCTGCCGGGTAAGCTGGCCGACTGCTCCTCCAAGGA |
| | | TGCATCGATCAGTGAACTATACATTGTCGAAGGTGACTCTGCG |
| | | GGTGGATCGGCCAAGCAAGGCCGGGACCGTCATTTCCAGGCCA |
| | | TCCTGCCGCTGCGCGGTAAGATCCTCAATGTAGAGAAGGCTCG |
| | | GCTTGACCGGATCTTGTCCAATGCGGAAATACGGGCCATTATC |
| | | ACAGCGCTCGGAACCGGGATCAGCGATGATTTCGACCTCTCTA |
| | | AGGCACGTTACCACAAGGTTGTCATCATGACCGATGCGGATGT |
| | | TGACGGTGCCCACATCAGAACACTGCTGCTGACTTTCTTTTAC |
| | | CGCTACATGCGGAAGATTGTCGATGCCGGGTATATCTTCATCG |
| | | CCCAGCCGCCGCTGTTCAAGATTGAACGCAATAAGGTTATCCG |
| | | CTATGCGCAGACCGAGAAGGAACGCGATGAGATTATCGCCTCC |
| | | TTCGGTGAGAACGTGAAGGTTAACGTTCAGCGCTACAAAGGTT |
| | | TGGGTGAGATGAATGCTGCCCAGCTCTGGGATACAACGATGGA |
| | | TCCTGAGAGCCGCACTATGCTGCAGGTAACGATTGAGGATGCT |
| | | ATGCTCGCTGACAGTATCTTTGATACACTTATGGGCGATAACG |
| | | TCCAGCCAAGATATGAATTTATCCAGGAGCACGCCCATTCCGT |
| | | CAAAAATCTCGATATTTAG |
| 22 | MS4921 | ATGTCAATGAATCAACCGACATATGATGAGAGCCAGATTCAGG |
| | | TACTGGAAGGGCTGGAGGCAGTTCGGAAACGTCCCGGCATGTA |
| | | CATCGGTTCTACCAGCGCCAAAGGTCTGCATCATTTGGTCTGG |
| | | GAGGTTGTCGATAACAGTATCGATGAGGCGCTGGCAGGCTATT |
| | | GCGACCGGATTCAAGTGAAGATTCATGAGGATAACAGTGTTAC |
| | | AGTAATAGATAATGGACGGGGTATCCCTGTCGGTGAGAATGTG |
| | | AAGCTGAAGAAGTCGACGCTCGAAGTCGTCATGACGGTCCTGC |
| | | ATGCAGGGGGAAAATTCGGCGGCGGCGGATATAAAGTATCAGG |
| | | CGGTTTGCACGGTGTAGGGATCTCCGTCGTAAATGCCTTGTCC |
| | | GAGAAGGTTGTTGTGAACGTCAAGCGTGACGGCCACATCTACC |
| | | AGCAGGAATATAGACGCGGTGCGCCGCAGTATGATATCAAGAT |
| | | CGTTGGGGATACCGATGAGACAGGGACAACCACAACCTTTCTT |
| | | CCGGATCCGGAGATTTTCACCGAAACGACAGTGTTTGAATATA |
| | | CGACATTGCTTACACGCATTCGCGAATTAGCCTTTCTTAACAA |
| | | GGGTATTGAGCTTTCGCTGCTGGATGAACGCACCGGTGTGTCG |
| | | AATGTTTTCAAATACGAGGGCGGTATCGTTGAGTATGTAAAAT |
| | | ATCTGAACGAGAAAAAAGAAGCGCTGCACGAAGAACCAATCTA |
| | | CGTAGAAGGCTCCCGTGATATGATTGCGGTGGAAGTGGCTCTG |
| | | CAGTATAACGATTCCTATACAGAGAACATTTACTCTTTTGCCA |
| | | ATAATATTAATACCCATGAGGGCGGGACCCATGAATCCGGCTT |
| | | CAAAAGCGCATTAACGCGGATCATCAATGACTATGCACGTAAA |
| | | ACAGGAGTTATTAAGGACAGCAGCTCCAACCTTACCGGTGATG |
| | | ATGTGCGTGAAGGGCTGACGGCGATTATTTCCGTTAAGATTCC |
| | | TGAGCCGCAATTTGAAGGCCAAACCAAGACCAAGCTGGGCAAC |
| | | AGTGAAGTCCGCGGTGTTGTAGAATCTTTATTCGGAGAGAAGC |
| | | TGCAGGAGTTCCTGGAAGAGAATCCGGCGGTGTCCCGGCGTGT |
| | | GCTGGAGAAATCCATGCAGGCTTCCCGTGCCCGTGAAGCGGCC |
| | | CGCAAGGCCCGTGAACTGACCCGCCGCAAAAGTGCACTTGAAG |
| | | TAAGCGCCCTGCCGGGGAAGCTGGCTGACTGCTCCTCCAAGGA |
| | | TGCCTCCATCAGCGAGCTGTACATCGTCGAAGGGGACTCCGCA |
| | | GGCGGTTCTGCCAAGCAGGGCCGTGACCGTCATTTCCAGGCAA |
| | | TTCTGCCGCTGCGCGGCAAGATACTGAACGTTGAAAAAGCGCG |
| | | GCTTGACCGTATATTGTCCAATGCTGAAATCCGGGCGATTATC |
| | | ACCGCTCTGGGAACCGGTATCAGTGATGATTTCGATCTGTCCA |
| | | AAGCCCGTTACCATAAGGTTGTTATCATGACCGATGCGGATGT |
| | | CGACGGTGCCCATATCAGAACACTGCTGCTGACCTTCTTCTAC |
| | | CGCTACATGCGGAAGATTATCGAAGCGGGCTATATTTACATCG |
| | | CCCAGCCGCCGCTGTTTAAGATTGAGCGCAATAAGGTCGTCCG |
| | | CTATGCTCAGACCGAGAAGGAACGTGATGAGATTATTGCCAGC |
| | | TTCGGCGAGAATGTTAAAGTCAACGTTCAACGGTATAAAGGTC |
| | | TTGGGGAAATGAATGCGGCACAGCTGTGGGATACGACTATGGA |
| | | CCCTGAGAGCCGTATGATGCTGCAGGTCACCATCGAGGATGCG |
| | | ATTCTCGCTGACGGAATCTTTGATACGCTGATGGGCGACAATG |
| | | TCCAGCCACGATATGAATTCATTCAGGAGAATGCGAAGTACGT |
| | | TCGGAACCTGGATTTCTAA |
| 23 | MS2748 | TTAAATATCAAGATTTTTTACGTACTGAGCATTTTCTTCAATG |
| | | AAATTACGGCGTGGTTCTACTTTGTCGCCCATTAAAATTTCAA |
| | | ATGTCTCATCAGCTTCAATTGCATCTTGCAAGTTCACCTGAAG |
| | | AAGGGTACGGAACTCTGGATCCATTGTTGTTTCCCATAATTGT |
| | | TCAGGATTCATCTCTCCTAAACCTTTGTAACGCTGAAGGTTTG |
| | | GTTTTGCGCCTTCAGGGAAAGAAGCTAATACCTCTTCTAATTG |
| | | ACGATCGTTGTACGCGTACTCCACTTTTTTACCTTGTGAAACT |
| | | TTGTAAAGAGGCGGCTGAGCAATGTACACATATCCGTGCTCAA |
| | | TAATCTGTCTCATATAGCGATAGAAGAACGTTAGAAGAAGCGT |
| | | ACGAATATGCGCACCGTCTACGTCTGCATCTGTCATAATCACA |
| | | ATTTTATGGTAGCGGGCTTTCGAAATATCAAATCGTCACAATC |
| | | CGTCCGTTCCTAGAGCGGTATGATTGTACGAATTTCGTTATTA |
| | | GATAAAATTTATCTAAACGCGCTTTCTCTACGTTGATAATTTT |
| | | ACCACGTAAAGGCAAAATAGCTTGGAAATGACGGCTTCTTCCC |
| | | TGCTTAGCTGAACCTCCGGCAGAGTCACCCTCTACTACATAGA |
| | | GTTCGCTAATAGAAGGATCTTTTGATGAACAATCTGCTAATTT |
| | | ACCCGGTAAGTTTGAAATTTCAAGCGCGCTTTTACGGCGTGTA |
| | | AGCTCACGAGCTTTTTTAGCTGCCATTCTTGCTCTTGCAGCCA |
| | | TTAAACCTTTTTCAATTACCTTTTTCGCCACAATAGGGTTCTC |
| | | TAGCAAGTAAGTTTCTAAGTGTTCTGCAAACACAGAGTCAGTA |
| | | ATTGTTCTTGCTTCACTATTTCCCAGCTTTGTTTTTGTTTGTC |
| | | CTTCGAACTGCGGATCTGGGTGCTTAATAGAGATGATAGCTGT |
| | | AATTCCTTCACGAACATCTTCACCTGTTAGATTGGCGTCACTG |
| | | TCTTTAAATACGCTGTTTTTACGTGCATAGTCGTTAATTACAC |
| | | GCGTTAACGCTGTTTTAAATCCTGCTTCGTGCGTTCCACCTTC |
| | | ATATGTGTGAATATTGTTTGCAAAAGAATATAAATTACTTGTA |
| | | TAGCTATCGTTATATTGAATAGCAATTTCTACAGAAATGTTGT |
| | | CTCGATTACCTTCAATATAGATTGGCTCTTCGTGAATCACTTC |
| | | TTTCGAACGGTTTAAGTGTTCAACGTAAGACTTAATTCCACCT |
| | | TCATAGTGATACTCACGTCTTTTATCTTCTTCACGTTTGTCTT |
| | | CAATCGTAATTTTAATGCCGCGATTTAAGAAAGCTAACTCACG |
| | | CAGACGATTAGCTAACGTATCAAAATCGTATTCAAGCGTTTCT |
| | | GTAAAAATTTCACCGTCTGGACGGAATTGAATAACAGTACCTG |
| | | TTTATCTGTTTCTCCAACTACTTTTAAGTCAGCAGCCGGTACA |
| | | CCTCGTTCATATTTTTGATAATGAACTTTACCGTCACGATGTA |
| | | CGTGCACTTCCAAAGAGGTAGAAAGTGCATTAACAACTGAGGC |
| | | ACCTACACCGTGTAATCCACCAGATACTTTATAGCCGCCGCCA |
| | | CCAAATTTACCTCCGGCATGAAGAACCGTTAAGATAACTTCAA |
| | | CGGCAGGTCTGCCATTTTTTCTTGAATACCAACCGGAATTCCA |
| | | CGACCGTTATCTTTGACTGTAATACTATTATCCTTTTCGATAA |
| | | TAACATTAATTTCATCGCAATAGCCGGCCAGCGCTTCATCAAT |
| | | ACTATTATCTACAATTTCCCATACAAGATGATGTAGACCCTTT |
| | | GCGCTCGTCGATCCAATATACATCCCCGGACGTTTACGAACAG |
| | | CTTCTAATCCTTCTAATACTTGTATCTGATCAGCTTCATATGC |
| | | TTGTACTTCTTTTTGTTCCATCGTCAA |

TABLE 9
Exemplary rpoB Gene Sequences of Bacterial Isolates

| SEQ ID | | |
|---|---|---|
| NO. | Bacterial Gene | Sequence |
| 24 | MS3900 | TTGACAGGTCAACTAGTTCAGTATGGACGCCACCGCCAACGCA |
| | | GAAGTTATGCTCGTATTAGTGAAGTTTTAGAGTTACCGAACTT |
| | | AATCGAGATTCAAACGGCTTCATACCAATGGTTTTTAGATGAA |
| | | GGTTTACGAGAAATGTTCCAAGATATTTCTCCAATTGATGATT |
| | | TTACAGGTAACTTATCACTAGAATTTATTGATTACAGCTTAGG |
| | | TGAGCCAAAGTATTCAGTAGGAGAGTCTAAAGAACGTGATGTA |
| | | ACTTATGCAGCACCTCTTCGTGTTAAGGTGCGGTTAATTAATA |
| | | AAGAAACAGGTGAAGTAAAAGACCAAGATGTGTTCATGGGAGA |
| | | TTTCCCATTGATGACCGAAACAGGTACCTTTGTAATTAACGGT |
| | | GCTGAGCGTGTTATCGTATCACAGTTGGTTCGTTCACCAAGTG |
| | | TTTACTATAGTGGAAAGCTTGATAAAAACGGTAAAAAAGGATA |
| | | TACAGCAACTGTTATCCCTAACCGTGGTGCATGGCTAGAATAT |
| | | GAAACTGATGCAAAAGATGTAGTATACGTGCGTATTGATCGTA |
| | | CTCGTAAACTGCCAGTAACAGTGCTGTTACGCGCGCTAGGTTT |
| | | CGGTTCTGACCAAGAGATTATCGATTTAATCGGTGATAATGAA |
| | | TACATCCGTAATACGCTTGAAAAAGATAATACGGAAACAACGG |
| | | AAAAAGCGCTATTAGAAATCTATGAGCGTTTACGTCCGGGTGA |
| | | ACCACCGACAGTTGAGAATGCGAAGTCTCTATTAGTATCTCGC |
| | | TTCTTTGATCCAAAACGATATGACTTAGCGAACGTAGGTCGCT |
| | | ATAAAATTAATAAAAAGCTTCATATCAAACATCGCTTATTTAA |
| | | TCAAAAGCTAGCTGAAACATTAGTTGATCCTGAAACAGGCGAA |
| | | ATTATTGCAGAAAAAGGCGCAATGATTGACCGCCGTCTGCTAG |
| | | ATCGCTTGATTCCGATGCTTGAAGGTGGAGTAAACTTCAAAAC |
| | | TTATAGTCCGGTTGGTGGAGTAGTAGAAGACGATGTTACATTA |
| | | CAATCTATTAAGATTTATGCGCCAAATGATCCAGAAGGTGAAA |
| | | AAATCATCACTGTATCAGGTAACGCATATGTAACAGAAGAAGT |
| | | TAAAAATATCACACCTGCTGATATTTTAGCATCAATCAGTTAC |
| | | TTCTTTAACTTGCTTCATCAAGTAGGAGACACAGATGATATCG |
| | | ACCACTTAGGTAACCGTCGTCTGCGTTCTGTAGGTGAATTGTT |
| | | ACAAAACCAATTCCGTATCGGTTTATCTCGTATGGAACGTGTT |
| | | GTTCGTGAAAGAATGTCAATTCAAGACACGAATACAATCACAC |
| | | CTCAACAATTAATTAATATTCGCCCAGTTATTGCGGCGATTAA |
| | | AGAGTTCTTTGGAAGTTCTCAATTATCACAGTTCATGGATCAA |
| | | ACGAATCCATTAGGCGAATTGACGCACAAACGTCGTCTTTCAG |
| | | CTCTAGGACCTGGTGGTTTAACGCGTGAGCGCGCTGGTTTCGA |
| | | AGTGCGTGACGTTCACTACTCCCACTATGGCCGTATGTGCCCG |
| | | ATTGAAACACCAGAGGGTCCGAATATCGGGTTAATCAACTCAC |
| | | TATCTTCTTATGCAAAAGTAAACAAATTCGGTTTCATCGAAAC |
| | | ACCTTACCGTCGTATCGATCCTGAAACAGGTAAAGTGACAGAG |
| | | CGAATTGACTACTTAACAGCTGATGAAGAAGATAACTATGTTG |
| | | TAGCCCAAGCGAACGCTCGTCTAGGTGATGATGGTTCATTCTT |
| | | AGATGAAAATGTCGTTGCACGTTTCCGTGGAGAAAACACGGTT |
| | | ATCCGTCGCGATCGTTTAGACTATATGGATGTATCACCAAAAC |
| | | AAGTTGTATCTGCCGCTACAGCATGTATCCCATTCTTAGAGAA |
| | | CGATGACTCTAACCGTGCATTAATGGGTGCGAACATGCAACGT |
| | | CAAGCAGTACCTTTATTAAATCCTGAAGCACCAATCGTAGGTA |
| | | CAGGTATGGAATACGTATCTGGTAAAGACTCTGGTGCAGCCGT |
| | | GATTTGTAAATATCCTGGCGTTGTAGAGCGCGTAGAAGCAAAA |
| | | CAAATTTTTGTTCGCCGCTATGAAGAAGTAGACGGACAAAAAG |
| | | TTAAAGGTAACTTAGATCAATACAAATTATTAAAATTTGTTCG |
| | | TTCTAACCAAGGTACTTGTTACAACCAGCGTCCAATTGTTTCA |
| | | GTTGGCGACGAAGTAGTAAAAGGTGAGATCTTAGCCGACGGTC |
| | | CTTCAATGGAAAAAGGTGAGCTTGCTTTAGGACGAAACGTAAT |
| | | GGTTGGTTTCATGACATGGGATGGTTACAACTATGAGGATGCC |
| | | ATCATCATGAGTGAACGCCTTGTGAAAGACGATGTATATACGT |
| | | CTGTTCATATTGAAGAATATGAATCTGAGTCTCGTGATACGAA |
| | | GCTTGGACCTGAAGAAATTACGCGTGACATTCCAAACGTAGGT |
| | | GAAGATGCGCTTCGCAACTTAGATGAGCGTGGAATCATCCGCA |
| | | TTGGTGCAGAAGTAAAAGACGGAGATCTTTTAGTTGGTAAAGT |
| | | AACGCCAAAAGGTGTAACAGAACTAACAGCTGAAGAACGTCTT |
| | | CTACACGCTATTTTCGGTGAAAAAGCGCGTGAAGTTCGTGATA |
| | | CTTCTCTTCGTGTACCGCACGGCGGCGGTGGAATCATTCTTGA |
| | | TGTTAAAGTCTTCAACCGTGAAGATGGGGACGAATTACCACCA |
| | | GGTGTAAACCAATTAGTCCGTGTATATATTGTTCAGAAGCGTA |
| | | AAATTTCTGAAGGTGACAAAATGGCCGGTCGTCACGGTAACAA |
| | | AGGTGTAATTTCACGTATTTTACCTGAAGAAGATATGCCTTAC |
| | | CTACCAGACGGTACGCCAATTGACATCATGTTAAACCCATTAG |
| | | GGGTACCATCTCGTATGAACATCGGTCAGGTGCTAGAGCTTCA |
| | | TTTAGGTATGGCTGCTCGTAAGCTTGGCATTCACGTTGCGTCT |
| | | CCAGTATTTGATGGTGCGCGTGAGGAAGATGTTTGGGCAACAA |
| | | TCGAAGAAGCTGGCATGTCTCGTGATGCTAAAACAGTTCTATA |
| | | TGATGGTCGAACAGGTGAACCATTCGATAACCGTGTATCAGTA |
| | | GGAATCATGTACATGATCAAACTTGCTCACATGGTAGACGATA |
| | | AACTTCACGCTCGTTCTACTGGACCATACTCACTTGTTACACA |
| | | ACAACCACTTGGTGGTAAAGCGCAGTTCGGTGGACAGCGTTTT |
| | | GGTGAGATGGAGGTATGGGCACTTGAAGCATACGGTGCTGCTT |
| | | ACACATTACAAGAGATCTTAACAGTGAAATCAGATGACGTAGT |
| | | AGGTCGTGTGAAAACATACGAAGCAATTGTTAAAGGTGAAAAC |
| | | ATTCCAGAACCTGGCATACCTGAATCGTTCAAAGTATTAATTA |
| | | AAGAACTACAAAGTTTAGGTATGGATGTGAAGATGCTTTCTGC |
| | | TGACGAGCAAGAAATTGATATCATGGACTCAGAAGAGGACCAT |
| | | GAGCAACCAACAGAATCAATTATTGCAGATAACGAAGAAAGCC |
| | | TTTCTGAAGGACAAAAAGATCCTGTCACAAAAGAGTAA |
| 25 | MS3907 | TTGCGGGAAATGTTCCAGGACATCTCGCCGATCCAGGATTTCA |
| | | CAGGTAATTTGGTACTAGAGTTCATTGATTACAGCCTGGGTGA |
| | | ACCGAAGTATACGGTTGACGACGCTAAAGAGCGGGACGTAACA |
| | | TATGCGGCTCCTCTGCGTGTGAAGGTGCGTCTCATCAATAAGG |
| | | AGACCGGTGAGGTCAAAGAGCAGGAAGTGTTCATGGGAGATTT |
| | | CCCTCTGATGACGGAGACCGGCACTTTTATTATCAATGGTGCG |
| | | GAACGGGTTATTGTCAGCCAGTTGGTTCGCTCTCCAAGCGTCT |
| | | ATTTCAGCACGAAAGTGGATAAGAACGGCAAAAAAACCTACAC |
| | | CGCCACAGTAATTCCGAATCGCGGAGCTTGGCTGGAGCTGGAG |
| | | ACCGACGCTAAGGACATCATGTATGTCCGTATCGACCGGACTC |
| | | GTAAGATCCCGGTTACCGTGCTTCTGCGTGCTCTAGGCTTCGG |
| | | CAGTGATGCTGAAATTCTGGAACTGCTTGGTAATGATGAATAT |
| | | ATTCGCAATACGCTGGATAAAGACAACACGGACTCTACGGAGA |
| | | AGGCGCTTATCGAAATTTACGAGCGTCTGCGTCCGGGCGAACC |
| | | ACCGACACTTGACAATGCCAAGAGCCTTTTGGTCGCACGTTTC |
| | | TTTGATCCGAAACGTTATGATTTGGCCAATGTAGGCCGTTACA |
| | | AAATCAACAAAAAGCTGCATATTAAAAATCGTCTGTTCAATCA |
| | | GCGTCTGGCACAACCTTTGGTGGATGAGTCTACTGGAGAAATC |
| | | CTGGCAGAATCCGGCCAAATGGTTGACCGCCGCCTGCTTGATG |
| | | AGCTGATTCCTTATTTTGAGAAGAACGTAGCTGCCAAGAACTA |
| | | CCGTGTAACCGGTGGGGTTATGGACAGCGAAGATATTCCGCTT |
| | | CAGACGATTGATGTGTTCTCGCCAATTGAAGAAGGCCGGATTA |
| | | TCAAACTGATCGCCAATGGCAACATTGACAAGTCGGTCAAGCA |
| | | TATTACTCAGGCTGATATTATATCCTCAATCAGCTACTTTATT |
| | | AATCTGCTGCACGGTATCGGCAACACTGATGATATCGACCACT |
| | | TGGGTAACCGCCGTCTGCGTTCTGTCGGCGAACTGCTGCAGAA |
| | | TCAGTTCCGTATCGGTCTGTCCCGCATGGAACGCGTTGTCCGC |
| | | GAGAGAATGTCGATTCAGGATGCCAATGCGATCACACCGCAGG |
| | | CGCTGATCAACATCCGCCCGGTTATCGCGTCGATTAAAGAGTT |
| | | CTTCGGTAGTTCACAGCTGTCCCAGTTCATGGATCAGACGAAC |
| | | CCGCTTGCTGAACTTACGCACAAACGCCGTCTATCGGCACTCG |
| | | GACCCGGCGGTTTGACCCGTGAACGCGCAGGCTTTGAAGTCCG |
| | | CGACGTCCATCACAGTCACTATGGCCGTATGTGTCCAATCGAA |
| | | ACACCGGAAGGTCCGAATATCGGTCTGATCAACTCCTTGTCCA |
| | | CCTTTGCCCGCATCAATGAATACGGCTTTATCGAAGCACCGTA |
| | | TCGTTGGGTGGATCCAAAGACAGGCAAAGTCACTGAGCAAATT |
| | | GATTATCTGACTGCCGATGAAGAAGATAACTATGTAGTTGCAC |
| | | AGGCAAATGTACTGATCGATGAGAATGGCTCCTTCAAGGAAGA |
| | | CCAGGTTATCGTTCGTTATAACAAAGATTCAGACAACATCACT |
| | | ACAATGCCAAGTAACCGTGTTGACTACATGGACGTTTCGCCTA |
| | | AACAGGTTGTATCAGTAGCTACGGCGCTCATTCCGTTCCTTGA |
| | | GAACGATGACTCCAACCGCGCGCTGATGGGATCGAACATGCAG |
| | | CGTCAGGCTGTTCCGCTTCTCATTCCGAAGGCTCCGCTTGTAG |
| | | GAACAGGGATGGAACATAAATCCGCTAAGGACTCGGGCGTATG |
| | | TATTGTCTCCAAATATGACGGTATTATCGAACGCTCCTCTGCC |
| | | AATGAAATCTGGCTGCGCCGTGTTGAGGCAGTTGAAGGCAAAG |
| | | AAGTTAAAGGCGATATCGTTAAATATAAATTACACAAATTTAT |
| | | GCGTTCGAACCAGGGTACCTGCATTAATCAGCGTCCGCTAGCC |
| | | AAACGCGGCGACGTTGTTAAGAAAGGTGATATCCTTGCAGACG |
| | | GACCATCTACGGAAATGGGCGAACTTGCTCTTGGCCGTAACGT |
| | | AGTTGTTGCGTTCATGACTTGGGAAGGCTACAACTACGAGGAT |
| | | GCGATCCTGCTGAGTGAGAAGCTGGTTAAGGAAGATGTATACA |
| | | CTTCGATCCATATCGAGGAATACGAATCCGAAGCCCGTGACAC |
| | | GAAGCTTGGACCTGAAGAAATCACGCGTGATATTCCAAACGTA |
| | | GGGGAAGAAGCGCTTCGCAATCTCGATGAGCGCGGAATTATCC |
| | | GGATCGGTGCGGAAATCAATGCCGGCGACATTCTGGTAGGTAA |
| | | AGTTACTCCGAAGGGCGTAACCGAGCTGACCGCTGAAGAACGT |
| | | CTGCTGCACGCTATCTTTGGTGAGAAGGCTCGTGAAGTCCGCG |
| | | ATACCTCTTTGCGCGTTCCACATGGTAGTGATGGTATTATCGT |
| | | TGACGTTAAGGTGTTCACGCGTGAGAACGGCGATGAGCTGCCT |
| | | CCAGGTGTGAATCAGCTGGTTCGTGTCTATATCGCTCAGAAAC |
| | | GTAAGATTTCTGAGGGTGACAAGATGGCCGGACGTCACGGTAA |
| | | CAAGGGTGTCGTTGCCCGTATCCTGCCTGAAGAAGATATGCCG |
| | | TTCCTTCCGGACGGTACACCGGTACAGGTTGTCCTGAACCCGC |
| | | TGGGCGTACCTTCCCGTATGAACATCGGACAGGTGCTTGAAGT |
| | | CCATCTCGGTATGGCTGCACTGCGTCTGGGTATTCACGTGGCT |
| | | ACTCCAGTATTTGACGGAGCCCGTGAGTATGACGTGTTTGATA |
| | | CGATGGAAGAAGCCGGTATGCAGCGTAACGGTAAAACTGTGCT |
| | | TTATGACGGACGTACAGGCGAACGCTTTGAGCGTGAAGTTACT |
| | | GTCGGCGTCATGCACATGATCAAGCTCGCGCACATGGTTGACG |
| | | ATAAAATTCATGCCCGTTCTACAGGTCCTTACTCTCTCGTTAC |
| | | ACAGCAGCCACTGGGCGGTAAGGCTCAGTTCGGCGGACAGCGT |
| | | TTCGGGGAAATGGAAGTGTGGGCGCTTGAAGCTTACGGCGCGG |
| | | CATATACACTGCAAGAAATCTTGACCGTGAAGTCCGATGACGT |
| | | GGTCGGCCGTGTGAAAACGTACGAGTCCATTGTCAAAGGCGAA |
| | | AATGTTCCAGAACCGGGTGTTCCGGAATCCTTCAAGGTATTGA |
| | | TCAAGGAACTGCAGTCGCTGGGTATGGATGTTAAGATCCTTAG |
| | | CGGTGACGAGCAGGAGATTGAGATGAAGGAACTGGACGATGAG |
| | | GACGAGACGTCAGGCGATAAGCTGAGCCTCAATTTGGAAGGCG |
| | | CAGAAGTCGGCATAGAGTAG |
| 26 | MS4921 | TTGCGTGAAATGTTTCAGGACATCTCGCCAATCCAGGATTTCA |
| | | CAGGGAATTTGGTACTTGAGTTCATTGATTACAGCCTAGGCGA |
| | | ACCGAAGTATACGGTTGACGACGCTAAAGAGCGGGACGTAACA |
| | | TATGCGGCTCCTCTACGTGTGAAGGTGCGGCTCATTAATAAGG |
| | | AGACCGGTGAGGTCAAAGAGCAGGAAGTGTTCATGGGAGATTT |
| | | CCCGCTGATGACGGAGACCGGCACTTTTATTATCAATGGTGCG |
| | | GAACGGGTTATTGTCAGCCAGTTGGTTCGCTCTCCAAGCGTCT |
| | | ATTTCAGCACGAAAGTAGATAAGAACGGCAAAAAAACCTACAC |
| | | CGCCACAGTAATTCCAAACCGCGGAGCCTGGCTTGAACTGGAG |
| | | ACCGACGCTAAGGATATCATGTATGTCCGTATCGACCGGACCC |
| | | GTAAAATTCCAGTTACAGTGCTTCTGCGTGCTCTCGGTTTCGG |
| | | CAGTGATGCTGAAATTCTGGAACTGCTGGGTAATGATGAATAT |
| | | ATTCGCAATACGCTGGATAAAGACAATACGGACTCCACGGAGA |
| | | AGGCGCTTATTGAAATTTACGAGCGTCTGCGTCCGGGTGAACC |
| | | GCCGACACTGGATAATGCCAAGAGCCTTTTGGTTGCACGTTTC |
| | | TTCGATCCAAAGCGTTATGACTTGGCCAATGTAGGCCGTTACA |
| | | AAATCAATAAGAAGCTGCACATTAAGAACCGGTTGTTTAACCA |
| | | GCGTCTGGCACAGCCTTTGGTTGATGAATCCACTGGGGAAATT |
| | | CTGGCGGAATCCGGACAAATGGTTGACCGCAGACTTCTTGATG |
| | | AGCTGATTCCTTATTTTGAGAAGGATGTCGCTGCCAAGACCTA |
| | | CCGTGTTACCGGCGGAGTGCTGGACAGTGAAGATATCCCGCTG |
| | | CAAACGATCGATGTATTCTCGCCGATCGAGGAAGGCCGGGTTA |
| | | TCAAGCTGATTGCCAACGGCAATATCGACAAATCCGTTAAGCA |
| | | TATTACCCAGGCTGATATTATATCCTCAATCAGCTACTTTATT |
| | | AATCTGCTGCACGGTATCGGCAACACAGACGACATTGACCACT |
| | | TGGGTAACCGCCGTCTGCGTTCCGTAGGTGAATTGCTGCAGAA |
| | | CCAATTCCGCATCGGGCTGTCCCGTATGGAACGCGTAGTGCGT |
| | | GAGAGAATGTCGATTCAGGATGCGAATGCAATTACTCCGCAGG |
| | | CCCTGATCAATATTCGTCCGGTCATCGCGTCCATTAAAGAGTT |
| | | CTTCGGCAGCTCCCAGCTGTCGCAGTTTATGGACCAGACGAAC |
| | | CCGCTGGCAGAACTTACGCATAAGCGTCGTCTGTCTGCACTCG |
| | | GACCCGGCGGTCTGACCCGTGAACGCGCGGGCTTTGAAGTTCG |
| | | GGACGTGCATCACAGTCACTACGGCCGGATGTGTCCAATCGAA |
| | | ACACCGGAAGGTCCGAATATCGGTCTGATCAACTCCTTGTCCA |
| | | CTTTTGCCCGCATCAATGAATACGGCTTTATCGAAGCTCCGTA |
| | | CCGTTGGGTGGATCCGAAGACCGGCAAGGTCACAGAGCATATC |
| | | GATTATCTGACCGCCGATGAAGAAGACAACTATGTAGTTGCGC |
| | | AGGCAAACGTGCAGATTGATGAAGATGGAACCTTTAAGGAAGA |
| | | TATGGTTATCGTCCGTTACAACAAGGATTCAGACAACATCACG |
| | | ACTATGCCTAGTAACCGTGTTGACTACATGGACGTTTCGCCAA |
| | | AACAGGTTGTATCGGTCGCTACGGCGCTCATTCCGTTCCTGGA |
| | | GAACGATGACTCCAACCGCGCACTGATGGGATCTAACATGCAG |
| | | CGCCAAGCCGTTCCGCTTCTGATTCCGAAGGCTCCGCTTGTCG |
| | | GTACAGGAATGGAGCATAAGTCCGCTAAAGACTCCGGCGTATG |
| | | TATTGTCTCCAAATATGACGGTATTATTGAACGCTCTTCTGCC |
| | | AATGAGATCTGGCTGCGCCGGGTTGAAATGGTTGAAGGCAAAG |
| | | AAGTCAAAGGTGATATCGTTAAATATAAATTACACAAATTTAT |
| | | GCGTTCGAACCAAGGGACTTGCATAAATCAGCGTCCGCTTGCT |
| | | AAAAGAGGCGACATTGTGAAGAAGGGTGACATTCTTGCGGATG |
| | | GTCCTTCCACCGAAATGGGCGAGTTGGCTCTGGGCCGCAACGT |
| | | AGTTGTTGCCTTTATGACTTGGGAAGGTTACAACTACGAGGAT |
| | | GCGATCCTGCTGAGTGAGAAGCTGGTGAAGGAAGATGTATACA |
| | | CTTCGATTCATATCGAGGAATACGAATCCGAAGCTCGTGACAC |
| | | GAAGCTGGGACCTGAAGAAATTACCCGTGATATTCCAAATGTC |
| | | GGTGAAGAAGCACTCCGTAACTTGGACGAACGCGGTATCATCC |
| | | GCATTGGTGCGGAAATCAATGCCGGTGACATCCTTGTAGGTAA |
| | | GGTAACTCCTAAAGGTGTAACTGAACTGACTGCTGAAGAACGT |
| | | CTGCTCCATGCAATCTTCGGGGAAAAAGCGCGTGAAGTTCGCG |
| | | ATACCTCCTTGCGTGTTCCGCATGGTAGTGATGGTATTATCGT |
| | | TGACGTCAAAGTATTCACACGTGAGAACGGCGATGAGCTGCCT |
| | | CCGGGTGTAAATCAGCTGGTTCGTGTCTACATCGCCCAGAAAC |
| | | GTAAAATTTCTGAGGGTGACAAAATGGCCGGACGTCACGGTAA |
| | | CAAGGGTGTCGTTGCCCGTATTCTTCCTGAGGAAGATATGCCA |
| | | TTCCTGCCGGACGGCACACCGGTACAGGTAGTATTGAACCCGC |
| | | TGGGCGTTCCTTCCCGTATGAACATCGGACAGGTGCTGGAGGT |
| | | CCATCTTGGTATGGCCGCTCTTCGTCTGGGCATTCATGTAGCT |
| | | ACTCCGGTATTTGACGGTGCCCGCGAGTATGACGTCTTTGATA |
| | | CGATGGAAGAAGCCGGCATGCAGCGCAATGGTAAGACTGTGCT |
| | | GTACGACGGACGTACAGGCGAACGCTTCGAGCGTGAAGTAACA |
| | | GTCGGTGTCATGCACATGATCAAGCTGGCGCACATGGTTGATG |
| | | ATAAGATTCATGCCCGTTCCACAGGTCCTTACTCACTCGTTAC |
| | | CCAGCAGCCTCTCGGCGGTAAAGCCCAGTTTGGTGGACAACGT |
| | | TTCGGGGAAATGGAAGTATGGGCGCTTGAAGCTTACGGCGCTG |
| | | CTTATACACTGCAGGAAATCTTGACGGTGAAATCCGATGACGT |
| | | GGTTGGCCGTGTGAAGACGTATGAATCCATCGTCAAAGGCGAG |
| | | AATCTGCCGGAACCTGGCGTGCCTGAAGCGTTCAAAGTATTGA |
| | | TCAAGGAACTGCAGTCGCTTGGTATGGATGTCAAAATCCTCAG |
| | | CGGTGATGAGCAGGAGATTGAGATGAAAGAACTGGACGATGAG |
| | | GACGAGACGACAGGCGACAAGCTGAGCCTTAACTTGGAAGGCG |
| | | CGGAAGTCGGAATAGAGTAG |
| 27 | MS2748 | TTACTCTTTTGTGACAGGATCTTTTTGTCCTTCAGAAAGACTT |
| | | TCTTCGTTATCTGCAATAATTGATTCTGTTGGTTGCTCATGGT |
| | | CCTCTTCTGAGTCCATGATATCAATTTCTTGCTCGTCAGCAGA |
| | | AAGCATCTTCACATCCATACCTAAACTTTGTAGTTCTTTAATT |
| | | AATACTTTGAACGATTCAGGTATGCCAGGTTCTGGAATGTTTT |
| | | CACCTTTAACAATTGCTTCGTATGTTTTCACACGACCTACTAC |
| | | GTCATCTGATTTCACTGTTAAGATCTCTTGTAATGTGTAAGCA |
| | | GCACCGTATGCTTCAAGTGCCCATACCTCCATCTCACCAAAAC |
| | | GCTGTCCACCGAACTGCGCTTTACCACCAAGTGGTTGTTGTGT |
| | | AACAAGTGAGTATGGTCCAGTAGAACGAGCGTGAAGTTTATCG |
| | | TCTACCATGTGAGCAAGTTTGATCATGTACATGATTCCTACTG |
| | | ATACACGGTTATCGAATGGTTCACCTGTTCGACCATCATATAG |
| | | AACTGTTTTAGCATCACGAGACATGCCAGCTTCTTCGATTGTT |
| | | GCCCAAACATCTTCCTCACGCGCACCATCAAATACTGGAGACG |
| | | CAACGTGAATGCCAAGCTTACGAGCAGCCATACCTAAATGAAG |
| | | CTCTAGCACCTGACCGATGTTCATACGAGATGGTACCCCTAAT |
| | | GGGTTTAACATGATGTCAATTGGCGTACCGTCTGGTAGGTAAG |
| | | GCATATCTTCTTCAGGTAAAATACGTGAAATTACACCTTTGTT |
| | | ACCGTGACGACCGGCCATTTTGTCACCTTCAGAAATTTTACGC |
| | | TTCTGAACAATATATACACGGACTAATTGGTTTACACCTGGTG |
| | | GTAATTCGTCGCCATCTTCACGGTTGAAGACTTTAACATCAAG |
| | | AATGATTCCACCGCCGCCGTGCGGTACACGAAGAGAAGTATCA |
| | | CGAACTTCACGCGCTTTTTCACCGAAAATAGCGTGTAGAAGAC |
| | | GTTCTTCAGCTGTTAGTTCTGTTACACCTTTTGGCGTTACTTT |
| | | ACCAACTAAAAGATCTCCGTCTTTTACTTCTGCACCAATGCGG |
| | | ATGATTCCACGCTCATCTAAGTTGCGAAGCGCATCTTCACCTA |
| | | CGTTTGGAATGTCACGCGTAATTTCTTCAGGCCCAAGCTTCGT |
| | | ATCACGAGACTCAGATTCATATTCTTCAATATGAACAGACGTA |
| | | TATACATCGTCTTTCACAAGGCGTTCACTCATGATGATGGCAT |
| | | CCTCATAGTTGTAACCATCCCATGTCATGAAACCAACCATTAC |
| | | GTTTCGTCCTAAAGCAAGCTCACCTTTTTCCATTGAAGGACCG |
| | | TCGGCTAAGATCTCACCTTTTACTACTTCGTCGCCAACTGAAA |
| | | CAATTGGACGCTGGTTGTAACAAGTACCTTGGTTAGAACGAAC |
| | | AAATTTTAATAATTTGTATTGATCTAAGTTACCTTTAACTTTT |
| | | TGTCCGTCTACTTCTTCATAGCGGCGAACAAAATTTGTTTTGC |
| | | TTCTACGCGCTCTACAACGCCAGGATATTTACAAATCACGGCT |
| | | GCACCAGAGTCTTTACCAGATACGTATTCCATACCTGTACCTA |
| | | CGATTGGTGCTTCAGGATTTAATAAAGGTACTGCTTGACGTTG |
| | | CATGTTCGCACCCATTAATGCACGGTTAGAGTCATCGTTCTCT |
| | | AAGAATGGGATACATGCTGTAGCGGCAGATACAACTTGTTTTG |
| | | GTGATACATCCATATAGTCTAAACGATCGCGACGGATGACCGT |
| | | GTTTTCTCCACGGAAACGTGCAACGACATTTTCATCTAAGAAT |
| | | GAACCATCATCACCTAGACGAGCGTTCGCTTGGGCTACAACAT |
| | | AGTTATCTTCTTCATCAGCTGTTAAGTAGTCAATTCGCTCTGT |
| | | CACTTTACCTGTTTCAGGATCGATACGACGGTAAGGTGTTTCG |
| | | ATGAAACCGAATTTGTTTACTTTTGCATAAGAAGATAGTGAGT |
| | | TGATTAACCCGATATTCGGACCCTCTGGTGTTTCAATCGGACA |
| | | CATACGGCCATAGTGGGAGTAGTGAACGTCACGCACTTCGAAA |
| | | CCAGCGCGCTCACGCGTTACCACCAGGTCCTAGAGCTGAAGAC |
| | | GACGTTTGTGCGTCAATCGCTAATGGATTCGTTTGATCCATGA |
| | | ACTGTGATAATTGAGAACTTCCAAAGAACTCTTTAATCGCCGC |
| | | AATAACTGGGCGAATATTAATTAATTGTTGAGGTGTGATTGTA |
| | | TTCGTGTCTTGAATTGACATTCTTTCACGAACAACACGTTCCA |
| | | TACGGGATAAACCGATACGGAATTGGTTTTGTAACAATTCACT |
| | | ACAGAACGCAGACGACGGTTACCTAAGTGGTCGATATCATCTG |
| | | TGTCTCCTACTTGATGAAGCAAGTTAAAGAAGTAACTGATTGA |
| | | TGCTAAAATATCAGCAGGTGAGATATTTTTAACTTCTTCTGTT |
| | | ACATATGCGTTACCTGATACAGTGATGATTTTTCACCTTCTGG |
| | | ATCATTTGGCGCATAAATCTTAATAGATTGTAATGTAACATCG |
| | | TCTTCTACTACTCCACCAACCGGACTATAAGTTTGAAGTTTAC |
| | | TCCACCTCAAGCATCGGAATCAAGCGATCTAGCAGACGGCGGT |
| | | CAATCATTGCGCCTTTTTCTGCAATAATTTCACCTGTTTCAGG |
| | | ATCAACTAATGTTTCAGCTAGCTTTTGATTAAATAAGCGATGT |
| | | TTGATATGAAGCTTTTTTATTAATTTTATAGCGACCTACGTTC |
| | | GCTAAGTCATATCGTTTTGGATCAAAGAAGCGAGATACTAATA |
| | | GAGACTTCGCATTCTCAACTGTCGGTGGTTCACCCGGACGTAA |
| | | ACGCTCATAGATTTCTAATAGCGCTTTTTCCGTTGTTTCCGTA |
| | | TTATCTTTTTCAAGCGTATTACGGATGTATTCATTATCACCGA |
| | | TTAAATCGATAATCTCTTGGTCAGAACCGAAACCTAGCGCGCG |
| | | TAACAGCACTGTTACTGGCAGTTACGAGTACGATCAATACGCA |
| | | CGTATACTACATCTTTTGCATCAGTTTCATATTCTAGCCATGC |
| | | ACCACGGTTAGGGATAACAGTTGCTGTATATCCTTTTTTACCG |
| | | TTTTTATCAAGCTTTCCACTATAGTAAACACTTGGTGAACGAA |
| | | CCAACTGTGATACGATAACACGCTCAGCACCGTTAATTACAAA |
| | | GGTACCTGTTTCGGTCATCAATGGGAAATCTCCCATGAACACA |
| | | TCTTGGTCTTTTACTTCACCTGTTTCTTTATTAATTAACCGCA |
| | | CCTTAACACGAAGAGGTGCTGCATAAGTTACATCACGTTCTTT |
| | | AGACTCTCCTACTGAATACTTTGGCTCACCTAAGCTGTAATCA |
| | | ATAAATTCTAGTGATAAGTTACCTGTAAAATCATCAATTGGAG |
| | | AAATATCTTGGAACATTTCTCGTAAACCTTCATCTAAAAACCA |
| | | TTGGTATGAAGCCGTTTGAATCTCGATTAAGTTCGGTAACTCT |
| | | AAAACTTCACTAATACGAGCATAACTTCTGCGTTGGCGGTGGC |
| | | GTCCATACTGAACTAGTTGACCTGTCAA |

Incubation of a microbial consortium in the bioreactor system may enrich a microbial community with plant growth promotion properties (e.g., nitrogen use efficiency-promoting microbes). Incubation of the inoculum of the microbe, the nitrogen use efficiency-promoting microbes of the microbial consortium, or any combination thereof may generate metabolites with desired plant growth promotion properties (e.g., nitrogen use efficiency-promoting metabolites. The inoculum of the microbe, nitrogen use efficiency-promoting microbes of the microbial consortium, nitrogen use efficiency-promoting metabolites, or any combination thereof may be nitrogen use efficiency-promoting microbes in the working fluid of the system and/or in the output product (e.g., base product) of the bioreactor system. A concentration of nitrogen use efficiency-promoting microbes may increase throughout a retention time of a bioreactor system described herein. The concentration of nitrogen use efficiency-promoting microbes may increase from a first container to a second container of the bioreactor system. The concentration of nitrogen use efficiency-promoting microbes may increase from a first container to a third container of the bioreactor system. The concentration of nitrogen use efficiency-promoting microbes may increase from a first container to a fourth container of the bioreactor system. The concentration of nitrogen use efficiency-promoting microbes may increase from a first container to a fifth container of the bioreactor system. The concentration of nitrogen use efficiency-promoting microbes may increase from a first container to a sixth container of the bioreactor system. In some embodiments, an output product (e.g., base product) may have a higher concentration of nitrogen use efficiency-promoting microbes than a concentration of nitrogen use efficiency-promoting microbes in a first container of a bioreactor system.

TABLE 10
Exemplary nifH gene sequences of bacterial isolates

| SEQ ID | Bacterial | |
|---|---|---|
| NO. | Gene | Sequence |
| 28 | MS3907 | ATGAGACAAATAGCTTTCTACGGTAAAGGCGGTATCGGCAAATCGACAACTTCA |
| | | CAAAACACCCTGGCTCAGCTGGCGACAAAGTTCGGACAAAGAATAATGATCGTA |
| | | GGCTGTGACCCTAAAGCAGACTCCACCCGCCTTATCCTGAATACAAAAGCCCAG |
| | | AACTCTGTGCTTGAACTGGCGGCTGAGCTTGGCTCGGTAGAGGATCTTGAACTT |
| | | GAAGATGTATTGCAGACAGGTTTCGGCGACATTATCAACGTAGAATGCGGCGGA |
| | | CCTGAACCGGGTGTAGGCTGCGCGGGACGCGGGATCATCACTGCCATCAACTTC |
| | | CTGGAGCAGGAAGGCGCCTATCAGGATCTGGATTTCGTATCCTATGACGTTCTT |
| | | GGTGACGTTGTATGCGGCGGTTTCGCAATGCCAATCCGCGAAGGCAAGGCGCAA |
| | | GAGATCTATATCGTCTGTTCCGGTGAAATGATGGCAATGTACGCAGCGAACAAT |
| | | ATCGCCCGCGGGATCCTGAAATATGCGACCAGCGGCGGCGTGAGACTGGGCGGC |
| | | CTGATCTGCAACAGCCGTAACACAGACCGTGAAGATGAGCTGATCATGGAACTC |
| | | GCCAGACGTCTGAACACGCAGATGATCCACTTTGTACCGCGTGACAATATCGTT |
| | | CAGCATGCCGAGCTGCGCAGAATGACTGTTGCCCAGTATAATCCGACCCATTCA |
| | | CAAGCGAAAGAATATGAAAAGCTGGCTGAGAAAATCCTCAATAACAAAATGCTG |
| | | ACGATCCCTACTCCGATTTCTATGGAAGAGCTGGAAGAGCTGCTGATGGAATTC |
| | | GGCATCATCGAAGACGAAGAGGCTGCAATCAAGAAGCTGCAGGCTTCCGGTCAA |
| | | TAA |
| 29 | MS4921 | ATGATGAGACAAATAGCTTTCTACGGTAAAGGCGGTATCGGTAAATCCACAACC |
| | | TCCCAAAACACTTTGGCCCAGCTCGCAACCAAATTCAAACAAAGAATTATGATC |
| | | GTAGGCTGTGACCCGAAGGCAGACTCCACCCGCCTGATTCTGAATACCAAGGCA |
| | | CAGAACTCGGTCCTGGAGCTGGCAGCCGAACTGGGCTCAGTAGAGGATTTGGAA |
| | | CTGGAGGATGTGCTGCAGACCGGCTTTGGCGACATTATCAACGTAGAGTGCGGC |
| | | GGACCTGAACCGGGTGTAGGCTGTGCAGGGCGCGGTATCATTACTGCCATCAAC |
| | | TTCCTGGAGCAGGAAGGCGCCTATCAGGATCTGGACTTCGTATCCTATGACGTA |
| | | TTGGGCGACGTTGTATGCGGCGGTTTCGCCATGCCGATCCGTGAAGGCAAAGCC |
| | | CAAGAGATCTATATTGTATGTTCCGGTGAAATGATGGCGATGTACGCGGCCAAC |
| | | AACATTGCACGCGGTATCCTGAAATATGCTACCAGCGGCGGCGTGAGACTGGGC |
| | | GGACTGATCTGCAACAGCCGCAACACCGACCGTGAAGACGAGCTGATCATGGAG |
| | | CTGGCCCGCCGTCTGAACACGCAAATGATCCACTTCGTTCCCCGTGACAATATC |
| | | GTTCAGCATGCCGAGCTGCGCAGAATGACGGTGGCCCAATATAACCCTGCCCAT |
| | | CAACAAGCCAAAGAATATGAAATTCTGGCTGAAAAAATCCTCAACAACAAAATG |
| | | CTGACCATCCCTACCCCGATTTCAATGGAAGAACTGGAAGAGCTGCTGATGGAA |
| | | TTCGGCATCATTGAAGATGAAGAAGCTGCACTCAAGAAGCTGCAGGCTTCCGGC |
| | | CAATAA |

In some embodiments, a concentration of nitrogen use efficiency-promoting microbes may increase from a first container to a second container, a third container, a fourth container, a fifth container, and/or a sixth container of a bioreactor system described herein. In some embodiments, a concentration of nitrogen use efficiency-promoting microbes in a second container, a third container, a fourth container, a fifth container, and/or a sixth container may be at least about 50×, at least about 100×, at least about 200×, at least about 250×, at least about 300×, at least about 400×, at least about 500×, at least about 600×, at least about 700×, at least about an 800×, at least about 900×, at least about 1000×, at least about 1250×, at least about 1500×, at least about 2000×, or greater than about 2000× increased from a concentration of nitrogen use efficiency-promoting microbes in a first container of a bioreactor system described herein. In some embodiments, a concentration of nitrogen use efficiency-promoting microbes in a second container, a third container, a fourth container, a fifth container, and/or a sixth container may be at most about 2000×, at most about 1500×, at most about 1250×, at most about 1000×, at most about 900×, at most about an 800×, at most about 700×, at most about 600×, at most about 500×, at most about 400×, at most about 300×, at most about 250×, at most about 200×, at most about 100×, at most about 50×, or less than about 50× increased from a concentration of nitrogen use efficiency-promoting microbes in a first container of a bioreactor system described herein.

In some embodiments, a concentration of nitrogen use efficiency-promoting microbes in a second container, a third container, a fourth container, a fifth container, and/or a sixth container may be from about 25× to about 2,500× increased from a concentration of nitrogen use efficiency-promoting microbes in a first container of a bioreactor system described herein. In some embodiments, a concentration of nitrogen use efficiency-promoting microbes in a second container, a third container, a fourth container, a fifth container, and/or a sixth container may be from about 25× to about 50×, about 25× to about 100×, about 25× to about 150×, about 25× to about 200×, about 25× to about 250×, about 25× to about 500×, about 25× to about 750×, about 25× to about 1,000×, about 25× to about 1,500×, about 25× to about 2,000×, about 25× to about 2,500×, about 50× to about 100×, about 50× to about 150×, about 50× to about 200×, about 50× to about 250×, about 50× to about 500×, about 50× to about 750×, about 50× to about 1,000×, about 50× to about 1,500×, about 50× to about 2,000×, about 50× to about 2,500×, about 100× to about 150×, about 100× to about 200×, about 100× to about 250×, about 100× to about 500×, about 100× to about 750×, about 100× to about 1,000×, about 100× to about 1,500×, about 100× to about 2,000×, about 100× to about 2,500×, about 150× to about 200×, about 150× to about 250×, about 150× to about 500×, about 150× to about 750×, about 150× to about 1,000×, about 150× to about 1,500×, about 150× to about 2,000×, about 150× to about 2,500×, about 200× to about 250×, about 200× to about 500×, about 200× to about 750×, about 200× to about 1,000×, about 200× to about 1,500×, about 200× to about 2,000×, about 200× to about 2,500×, about 250× to about 500×, about 250× to about 750×, about 250× to about 1,000×, about 250× to about 1,500×, about 250× to about 2,000×, about 250× to about 2,500×, about 500× to about 750×, about 500× to about 1,000×, about 500× to about 1,500×, about 500× to about 2,000×, about 500× to about 2,500×, about 750× to about 1,000×, about 750× to about 1,500×, about 750× to about 2,000×, about 750× to about 2,500×, about 1,000× to about 1,500×, about 1,000× to about 2,000×, about 1,000× to about 2,500×, about 1,500× to about 2,000×, about 1,500× to about 2,500×, or about 2,000× to about 2,500×.

In some embodiments, the absolute population of the microbial inoculum of the bioreactor system may not decrease by more than about 0.000001%, 0.00001%, 0.0001%, 0.001%, 0.01%, 0.1%, 0.5%, 1%, 2%, 3%, 4%, 5%, 6%, 7%, 8%, 9%, 10%, 15%, 20%, or 25% after incubation. In some embodiments, the absolute population of the microbial inoculum of the bioreactor system may not decrease by more than about 0.000001%, 0.00001%, 0.0001%, 0.001%, 0.01%, 0.1%, 0.5%, 1%, 2%, 3%, 4%, 5%, 6%, 7%, 8%, 9%, 10%, 15%, 20%, or 25% after incubation for a retention period of a bioreactor system (e.g., 1 day, 2 days, 3 days, 4 days, 5 days, 6 days, 1 week, 2 weeks, 3 weeks, 4 weeks, 2 months, 3 months, 4 months, 5 months, 6 months, 1 year, 2 years, 3 years, 4 years, or 5 years). In some embodiments, the absolute population of the microbial inoculum of the bioreactor system may not decrease by more than about 0.000001%, 0.00001%, 0.0001%, 0.001%, 0.01%, 0.1%, 0.5%, 1%, 2%, 3%, 4%, 5%, 6%, 7%, 8%, 9%, 10%, 15%, 20%, or 25% after incubation without inoculation of additional microbe.

In some embodiments, a proportion of a concentration of nitrogen use efficiency-promoting microbes relative to a total bacterial population count may increase across containers of a fluidly connected bioreactor system. In some embodiments, a proportion of a concentration nitrogen use efficiency-promoting microbes relative to a total bacterial population count may increase from a first container to a second container of a bioreactor system described herein. In some embodiments, a proportion of a concentration nitrogen use efficiency-promoting microbes relative to a total bacterial population count may increase from a first container to a third container of a bioreactor system described herein. In some embodiments, a proportion of a concentration nitrogen use efficiency-promoting microbes relative to a total bacterial population count may increase from a first container to a fourth container of a bioreactor system described herein. In some embodiments, a proportion of a concentration nitrogen use efficiency-promoting microbes relative to a total bacterial population count may increase from a first container to a fifth container of a bioreactor system described herein. In some embodiments, a proportion of a concentration nitrogen use efficiency-promoting microbes relative to a total bacterial population count may increase from a first container to a sixth container of a bioreactor system described herein.

Population of the microbe may be measured using methods including but not limited to spectrophotometers, cell counting, measures of turbidity, hemocytometers, electronic enumeration, determination of nitrogen content, measures of cell mass, quantitative polymerase-chain reaction (qPCR), semi-quantitative PCR, and measures of cell activity.

The addition of the target isolate (e.g., the inoculum of the microbial strain/microbe) can have an added benefit in improving target functionality of working solution in a reactor of the bioreactor system and/or in the output base product of the bioreactor system. In some embodiments, addition of the inoculum of the microbial strain can improve the nitrogen use efficiency-promoting solubilization capacity of working fluids and base product of a bioreactor system compared to working fluids and base product of a bioreactor system without an inoculum of the microbial.

A bioreactor system may be inoculated with a microbial strain at the start of a bioreactor system, which may allow for the microbial strain to flow through the system and working fluids of the reactors. Without wishing to be bound by theory, a bioreactor system inoculated with a microbial strain may increase a nitrogen use efficiency capacity of a working fluid and/or a base product compared to a working fluid and/or base product of an otherwise identical bioreactor system with no inoculated microbial strain. Without wishing to be bound by theory, a bioreactor system inoculated with a microbial strain may increase a nitrogen use efficiency capacity of a working fluid and/or a base product compared to a working fluid and/or base product of an otherwise identical bioreactor system with the microbial strain added (e.g., spiked) at the end of the system. Without wishing to be bound by theory, a bioreactor system inoculated with a microbial strain may increase a nitrogen use efficiency capacity of a base product compared to a base product of an otherwise identical bioreactor system with the microbial strain added (e.g., spiked) at the end of the system. Incubation of the microbial strain in the bioreactor system may enrich the working fluid with nitrogen use efficiency-promoting microbes and/or generate nitrogen use efficiency-promoting metabolites.

### Examples

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

The examples below are illustrative and non-limiting.

### Example 1: Exemplary Zinc-Solubilizing Bioreactor Systems

ZST batch systems (ZST 1.0 system) configured for production of zinc solubilization promoting products by digestion of organic feedstock were built and operated. The ZST 1.0 system was an up-flow system. The system operated as a batch process with one 5-gallon reactor. The hydraulic source for the ZST batch systems was water at an electrical conductivity ranging from 250-1000 microsiemens/centimeter (μS/cm). The water comprised water from a city water source and a reverse osmosis (RO) water source. In some cases, the working fluid in each reactor is recirculated from the bottom of the reactor and pumped back to just below the surface of the same reactor to maintain homogeneous conditions within the working solutions. Biosolids (floc) is generated through the process. The reactor of the ZST 1.0 system was a fluidized bed reactor, which lacked scaffolding. The ZST 1.0 systems were set up to closely replicate benchtop shake flask experiments. The batch reactors systems were used to understand the utilization and enrichment of target isolate MS4666 and were also used to understand the fermentation process regarding the loading inputs of the process and timings. The batch systems utilized the phosphate solubilizing technology (PST) floc as the microbial inoculum. A diagram of the ZST 1.0 system is shown in FIG. 1. A batch reactor system can have a plurality of inputs.

In the ZST 1.0 Batch reactor systems, zinc oxide was added into the batch reactor to maintain a concentration range between 0.1-1.0% w/v on the first day at the start of the batch process. A concentration of range of 0.1-5.0% v/v of PST floc of the phosphate solubilizing was also added the reactor on the first day of the start of the batch process. The PST WB indicated as an input into the reactor (in FIG. 1) refers to a product of a separate serialized bioreactor system (the PST system), which has as an input an aqueous feed comprising (1) a microbial consortium derived from digestion of cow manure by microbes endogenous to the manure and (2) rock phosphate.

Glucose and ammonium sulfate were added into the reactor to maintain a concentration range of 0.5-3.0% w/v and 0.05-0.2% w/v, respectively, on the first day of the batch process. In some cases the glucose and ammonium sulfate were added on the second day of the process or added in a later day based on the expected retention time of the batch process. Potassium chloride (KCl), magnesium sulfate (MgSO₄), and dipotassium phosphate (K₂HPO₄) were added into batch reactor at a concentration range of 0.01-0.05% w/v, 0.01-0.05% w/v, and 0.05-0.2% w/v, respectively, on the first day of the batch process. In some cases of the ZST 1.0 batch reactor systems, potassium chloride (KCl), magnesium sulfate (MgSO₄), and dipotassium phosphate (K₂HPO₄) were added on the second day of the process or may be an addition in later day based on the expected retention time of the batch process.

The pH within each reactor changes depending on the timing of feedstock addition and its consumption. In the ZST 1.0 reactor systems, the pH in the reactor may range between 4.00-9.00. The total volume of the working fluid for the batch system was maintained at 5 gallons. The retention time of the batch system was maintained at 7 days and varied accordingly between 7 to 21 days depending on production volume requirements. The working fluid in the reactor of the ZST 1.0 system was circulated within the reactor in such a way as to minimize surface disruption.

### ZST 1.5/1.5+System

Two separate ZST systems, ZST 1.5 and ZST 1.5+, for production of zinc solubilization promoting products by digestion of organic feedstock were built and operated. The ZST 1.5 system was the first continuous serialized system was constructed to investigate the enrichment of target isolate MS4666 within a complex consortium for a specific functionality. The inoculum of MS4666 was a liquid culture prepared using Tryptic Soy Broth (TSB, sigma 22092) that was added in the first reactor of the series once when the system was started. The system was a fluidized bed-based system that utilized PST floc as the microbial inoculum similar to the ZST 1.0 batch reactor systems. The microbial inoculum eventually was modified to a PST 1:1 floc: supernatant ratio as the microbial inoculum. The ZST 1.5 system comprised four 5-gallon reactors that used an up-flow orientation of the hydraulic re-circulation within each reactor and were followed by a clarifier from which the byproducts were recovered. The byproducts comprised: (1) the floc that was separated by gravity and (2) the supernatant (e.g., base product) that was collected as the main product output of the system. Floc that was recovered from the clarifier was returned manually upstream of the system to either Reactor 1 or Reactor 2. The ZST 1.5+ system was based on the ZST 1.5 system to further investigate the enrichment of the target isolate MS4666 based on the initial inoculation method. The inoculation of MS4666 was performed using a powder preparation of MS4666 (spray dried) instead of a liquid broth. Additionally, the system utilized PwST WB instead of PST at a 1:1 floc: supernatant ratio. The main objective of the line was to understand if there were any differences between the inoculation of the target isolate (either in liquid culture as used in the ZST 1.5 or powder) and to examine the differences using the PwST WB as the microbial inoculum. The ZST 1.5+ system comprised four 5-gallon reactors that used an up-flow orientation of the hydraulic re-circulation within each reactor and were followed by a clarifier from which the byproducts were recovered. The byproducts comprised: (1) the floc that was separated by gravity and (2) the supernatant (e.g., base product) that was collected as the main product output of the system. Floc that was recovered from the clarifier was returned manually upstream of the system to either Reactor 1 or Reactor 2. A diagram of the ZST 1.5/1.5+ system is shown in FIG. 5.

The ZST 1.5/1.5+serialized systems were operated with a continuous flow of input and from reactor to reactor. The continuous hydraulic source for the ZST serialized systems is water at an electrical conductivity ranging from 250-1000 microsiemens/centimeter (μS/cm). Working fluid continuously flowed from one reactor to the next and ultimately to the clarifier container, which was a 5 gallon reactor. Initially, a water tank was connected to Reactor 1 and provided continuous flow of water to Reactor 1. The water tank comprised water from a city water source and a reverse osmosis (RO) water source.

In the ZST 1.5 system, the pH in Reactor 1 ranged from 8.00-9.00 with an average pH of 8.89. Following the start of the fermentation in Reactor 2, the pH range was 4.00-8.00 with an average pH of 6.50, in Reactor 3 was 4.00-9.00 with an average pH of 6.30, and/or in Reactor 4 was 5.50-9.00 with an average pH of 6.62. The clarifier had a pH range of 5.00-7.00 with an average pH of 6.89. The base product had a pH range of 6.8-7.5 with an average pH of 7.11. In the ZST 1.5+ system, the pH in Reactor 1 ranged from 8.00-9.00 with an average pH of 8.66. Following the start of the fermentation in Reactor 2, the pH range was 4.00-8.00 with an average pH of 7.62, in Reactor 3 was 4.00-9.00 with an average pH of 7.42, and/or in Reactor 4 was 5.50-9.00 with an average pH of 7.84. The clarifier had a pH range of 5.00-8.00 with an average pH of 7.75. The base product had a pH range of 6.8-7.8 with an average pH of 7.56.

As shown in FIG. 5, zinc oxide was added into Reactor 1 to maintain a concentration range of 0.05-0.5% w/v, based on the span of the hydraulic retention time of the system. For the ZST 1.5 system, a concentration range of 0.5-8% v/v of whole broth of a Phosphate Solubilization Technology, PST WB (in a ratio of 1:1 floc: supernatant (SPN)) was also added to Reactor 1 on the span of the hydraulic retention time of the system. For the ZST 1.5+ system, a concentration range of 0.5-8% v/v of the whole broth of a water based phosphate solubilization technology, PwST WB (in a ratio of 1:1 floc: SPN) was also added to Reactor 1 on the span of the hydraulic retention time of the system. Glucose and ammonium sulfate were added into Reactor 2 to maintain a concentration range of 0.5-3.0% w/v and 0.05-0.2% w/v, respectively, based on the span of the hydraulic retention time of the system. Potassium chloride (KCl), magnesium sulfate (MgSO₄), and dipotassium phosphate (K₂HPO₄) were added into Reactor 2 at a concentration range of 0.01-0.05% w/v, 0.01-0.05% w/v, and 0.05-0.2% w/v, respectively, based on the span of the hydraulic retention time of the system. In some cases of the ZST 1.5 and ZST 1.5+ systems, potassium chloride, magnesium sulfate, and/or dipotassium phosphate were not added into the systems. Fluid from the top of the Reactor 2 was continuously flowed into Reactor 3 in a hydraulically balanced fashion. In some cases, fluid from the top of a third reactor was flowed into a separation chamber (e.g., the clarifier), where solids were allowed to settle. The supernatant (or base product) from the clarifier was continuously collected, and at least a portion of the floc at the bottom of the clarifier was returned to Reactor 1 at a rate of 2% v/v of the system total volume.

The ZST 1.5 and ZST 1.5+ systems were operated with a continuous flow of input and from reactor to reactor. The total volume of working fluid within all of the reactors and clarifiers of ZST 1.5 and ZST 1.5+ systems was maintained at 25 gallons. The total volume consisted of three 5-gallon reactors and one 5-gallon clarifier container. The hydraulic rate of flow through the bioreactor systems would be 4.69 mL/min to maintain a retention time of 14 days and varied accordingly for a retention time from 7 to 21 days depending on production volume requirements. The hydraulic flow rate varied between 3.13-9.39 mL/min. The ZST 1.5 and ZST 1.5+ systems produced 1.19 to 3.57 gallons of base product flowing from the clarifiers per day. The ZST 1.5 and ZST 1.5+ systems produced 8.33 to 25 gallons of base product flowing from the clarifiers per week.

The ZST digestion process incorporated an aerobic phase at the beginning of the system (e.g., in Reactor 1) and a microaerobic phase during the fermentation, stabilization, and clarification stages of the process. The working fluid in each reactor described herein was circulated within the reactor in such a way as to minimize surface disruption during the fermentation phase of the process. In the ZST 1.5 system, the dissolved oxygen in Reactor 1 ranged from 4.00-7.00 mg/L with an average of 6.43 mg/L, as it was high mix rate reactor. Following the start of the fermentation in Reactor 2, the dissolved oxygen range was 0.10-1.20 mg/L with an average of 0.18 mg/L, in Reactor 3 was 0.10-1.20 mg/L with an average of 0.15 mg/L, and/or in Reactor 4 was 0.15-1.20 mg/L with an average of 0.16 mg/L. In the ZST 1.5+system, the dissolved oxygen in Reactor 1 ranged from 4.00-7.00 mg/L with an average of 6.02 mg/L, as it is highly mixed reactor. Following the start of the fermentation in Reactor 2, the dissolved oxygen range was 0.10-1.20 mg/L with an average of 0.29 mg/L, in Reactor 3 was 0.10-1.20 mg/L with an average of 0.23 mg/L, in Reactor 4 was 0.15-1.20 mg/L with an average of 0.18 mg/L, and/or the clarifier was between 0.05-1.2 mg/L with an average of 0.22 mg/L.

### ZST 2.0 Systems

Two additional scaled up separate systems ZST 2.0 FBR and ZST 2.0 PBR (ZST 2.0 systems) were built for optimization at a larger scale.

For the ZST 2.0 FBR system, the inoculum of MS4666 was a liquid culture prepared using Tryptic Soy Broth (TSB, sigma 22092). The system was a fluidized bed-based system, similar to the ZST 1.5/1.5+ systems, that utilized PST WB at a 1:1 floc: supernatant ratio as the microbial inoculum. The orientation of the hydraulic re-circulation within each reactor was down-flow. The system comprised a fifth reactor at the end of the series before the clarifier as well as a complete mixed reactor (CMR) at the front end of the system acting as the Reactor 1 of ZST 1.5 where the MS4666 inoculum was incorporated once when the system was started. The distribution of the working volume of the system was not equally dispersed throughout the system. Floc-flights were incorporated in the clarifier to fold the floc, which assisted the release of MS4666 from the floc into the supernatant, and aided in the recirculation back upstream of the process (Reactor 1).

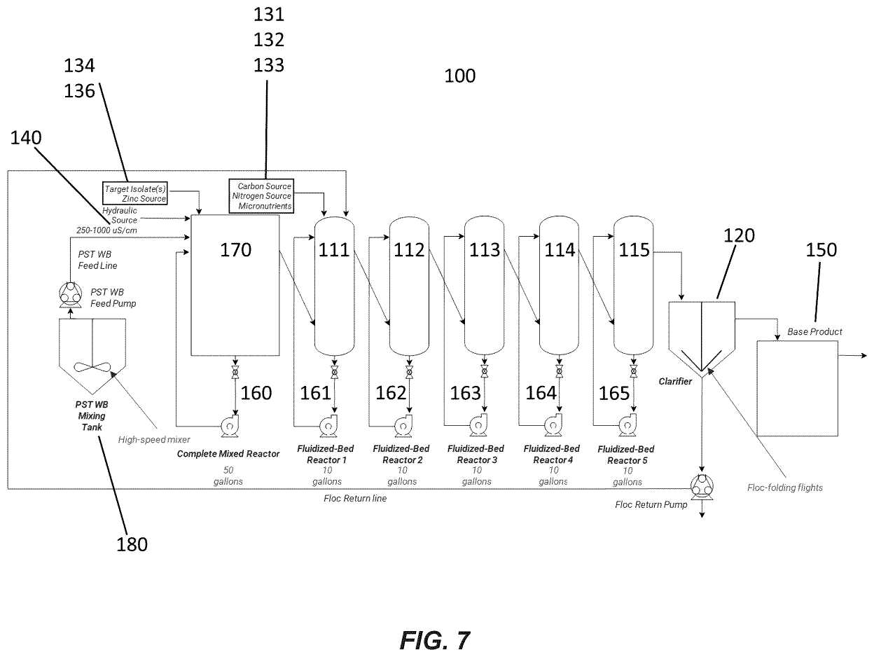

The ZST 2.0 PBR system was a parallel line constructed to compare the use of a packed bed reactor (PBR) configuration to the ZST 2.0 FBR (fluidized bed reactor) configuration described herein. Reactors 1-5 comprised scaffolding and the scaffolding was comprised to increase the surface area of each reactor potentially promoting better biofilm growth and retention of the isolate. Similarly, to the ZST 2.0 FBR, the inoculum of MS4666 was a liquid culture prepared using Tryptic Soy Broth (TSB, sigma 22092) that was added once to the CMR. The system used PST WB at a 1:1 floc: supernatant ratio as the microbial inoculum and the orientation of the hydraulic re-circulation within each reactor was down-flow. ZST 2.0 systems were the first systems to utilize PST WB at a 1:1 floc: supernatant ratio as the microbial inoculum. The distribution of the working volume of the system was not equally dispersed throughout the system. As a parallel line to the ZST 2.0 FBR, the ZST 2.0 PBR system incorporated floc-flights in the clarifier to fold the floc, helping the release of MS4666 from the floc into the supernatant, and to aid in the recirculation back upstream of the process (Reactor 1). A diagram of the ZST 2.0 FBR system is shown in FIG. 7 and a diagram of the ZST 2.0 PBR system is shown in FIG. 8.

The ZST 2.0 systems were operated with a continuous flow of input and from reactor to reactor. The continuous hydraulic source for the ZST serialized systems is water at an electrical conductivity ranging from 250-1000 microsiemens/centimeter (μS/cm). The total volume of working fluid within all the reactors and clarifiers of the ZST 2.0 FBR and ZST 2.0 PBR systems was maintained at 115 gallons. The hydraulic rate of flow through these two systems was 21.59 mL/min to maintain a retention time of 14 days and can be varied accordingly for a retention time from 7 to 21 days depending on production volume requirements with a hydraulic rate range between 14.40-43.19 mL/min. The retention time of the ZST1.5 and ZST1.5+ system, and/or the ZST 2.0 FBR and ZST 2.0 PBR systems was 14 days. In some embodiments of the ZST1.5 and ZST1.5+ systems, the hydraulic rate of flow through the bioreactor system may be 4.69 mL/min to maintain a retention time of 14 days and can be varied accordingly for a retention time from 7 to 21 days depending on production volume requirements. Because of this, the amount of product produced per day can range from 1.19 to 3.57 gallons. The amount of product produced per week can range from 8.33 to 25 gallons. In some embodiments of the ZST2.0 FBR and ZST2.0 PBR systems, the hydraulic rate of flow through the bioreactor system may be 21.59 mL/min to maintain a retention time of 14 days and can be varied accordingly for a retention time from 7 to 21 days depending on production volume requirements. Because of this, the amount of product produced per day can range from 5.48 to 16.43 gallons. The amount of product produced per week can range from 38.34 to 115.01 gallons. The ZST 2.0 FBR and ZST 2.0 PBR systems resulted in a 4.6× total system volume scale up. The scale up of ZST1.5 system to the ZST 2.0 FBR and ZST 2.0 PBR systems used retention time as the constant variable to determine scale factors.

In each of the ZST 2.0 FBR and ZST 2.0 PBR systems, zinc oxide was added into the CMR to maintain a concentration range of 0.05-0.5% w/v, based on the span of the hydraulic retention time of the system. A concentration range of 0.5-8% v/v of whole broth of a phosphate solubilization technology, PST WB (in a ratio of 1:1 floc: supernatant (SPN)) was also added to the CMR on the span of the hydraulic retention time of the system. PST WB was added from a PST WB mixing tank, which had a high-speed mixer. PST WB was inputted through a PST WB feed pump and corresponding feed line into the CMR of the ZST 2.0 FBR and ZST 2.0 PBR systems. Glucose and ammonium sulfate were added into Reactor 1 to maintain a concentration range of 0.5-3.0% w/v and 0.05-0.2% w/v, respectively, based on the span of the hydraulic retention time of the system. Potassium chloride (KCl), magnesium sulfate (MgSO₄), and dipotassium phosphate (K₂HPO₄) were added into Reactor 1 at a concentration range of 0.01-0.05% w/v, 0.01-0.05% w/v, and 0.05-0.2% w/v, respectively, based on the span of the hydraulic retention time of the system. In some cases, potassium chloride, magnesium sulfate, and/or dipotassium phosphate were not added into the systems. Fluid from the top of the Reactor 1 was continuously flowed into Reactor 2 in a hydraulically balanced fashion. Fluid from the top of the Reactor 2 was continuously flowed into Reactor 3, and from Reactor 3 to Reactor 4, and from Reactor 4 to Reactor 5, in a hydraulically balanced fashion. In some cases, fluid from the top of a fifth reactor was flowed into a separation chamber (e.g., clarifier), where solids were allowed to settle. The supernatant (e.g., base product) from the clarifier was continuously collected, and at least a portion of the floc at the bottom of the clarifier was returned to Reactor 1 at a rate of 2% v/v of the system total volume.

Similar to the ZST 1.5/1.5+ systems, the ZST digestion process of the ZST 2.0 systems incorporated an aerobic phase at the beginning of the system and a microaerobic phase during the fermentation, stabilization, and clarification stages of the process. In the ZST 2.0 FBR system, the dissolved oxygen in the CMR ranged from 4.00-7.00 mg/L with an average of 4.80 mg/L, as it is highly mixed reactor. Following the start of the fermentation in Reactor 1, the dissolved oxygen range was 0.02-1.20 mg/L with an average of 0.07 mg/L, in Reactor 2 was 0.02-1.20 mg/L with an average of 0.10 mg/L, in Reactor 3 was 0.05-1.20 mg/L with an average of 0.12 mg/L, in Reactor 4 was 0.5-2.6 mg/L, with an average of 2.05 mg/L, in Reactor 5 was 0.5-2.0 mg/L with an average of 1.05 mg/L, and/or the clarifier was between 0.20-1.2 mg/L with an average of 0.51 mg/L. In the ZST 2.0 PBR system, the dissolved oxygen in the CMR ranged from 4.00-9.00 mg/L with an average of 6.52 mg/L, as it is highly mixed reactor. Following the start of the fermentation in Reactor 1, the dissolved oxygen range was 0.02-1.20 mg/L with an average of 0.08 mg/L, in Reactor 2 was 0.02-1.20 mg/L with an average of 0.08 mg/L, in Reactor 3 was 0.05-1.20 mg/L with an average of 0.56 mg/L, in Reactor 4 was 0.5-2.6 mg/L, with an average of 1.54 mg/L, in Reactor 5 was 0.5-2.0 mg/L with an average of 0.11 mg/L, and/or the clarifier was between 0.05-1.2 mg/L with an average of 0.12 mg/L.

The total volume of working fluid within all the reactors and clarifiers of the ZST 2.0 FBR and ZST 2.0 PBR systems was maintained at 115 gallons. The volume of the CMR was 50 gallons, the volume of Reactors 1-5 was 10 gallons, and the volume of the clarifier was 15 gallons. The hydraulic rate of flow through these two systems was 21.59 mL/min to maintain a retention time of 14 days and varied accordingly for a retention time from 7 to 21 days depending on production volume requirements with a hydraulic rate range between 14.40-43.19 mL/min. The retention time of the ZST 2.0 FBR and ZST 2.0 PBR systems was 14 days. The ZST 2.0 FBR and ZST 2.0 PBR systems produced 5.48 to 16.43 gallons of base product flowing from the clarifiers per day. The ZST 2.0 FBR and ZST 2.0 PBR systems produced 38.34 to 115.01 gallons of base product flowing from the clarifiers per week.

In the ZST 2.0 FBR system, the pH in the CMR ranged from 7.20-9.00 with an average pH of 8.40. Following the start of the fermentation in Reactor 1, the pH range was 4.00-8.00 with an average pH of 6.78, in Reactor 2 was 4.00-9.00 with an average pH of 6.40, in Reactor 3 was 4.00-8.00 with an average pH of 6.11, in Reactor 4 was 5.50-8.00 with an average of 6.61, and/or in Reactor 5 was 5.50-8.00 mg/L with an average of 6.73. The clarifier had a pH range of 5.00-7.00 with an average pH of 6.81. The base product had a pH range of 6.8-8.00 with an average pH of 6.94. In the ZST 2.0 PBR system, pH in the CMR ranged from 7.20-9.00 with an average pH of 7.90. Following the start of the fermentation in Reactor 1, the pH range was 4.00-8.00 with an average pH of 6.47, in Reactor 2 was 4.00-9.00 with an average pH of 5.95, in Reactor 3 was 4.00-8.00 with an average pH of 6.39, in Reactor 4 was 5.50-8.00 with an average of 6.99, and/or in Reactor 5 was 5.50-8.00 mg/L with an average of 7.05. The clarifier had a pH range of 5.00-7.00 with an average pH of 7.46. The base product had a pH range of 6.8-8.00 with an average pH of 7.56.

### Summary of Zinc Solubilizing Systems

The pH value for each ZST system across the digestion process phases is shown in Table 11. The phases of the digestion process were the mixing phase, the zinc-solubilizer enrichment phase, the stabilization phase, the clarifier phase, and the product.

TABLE 11
pH Comparison Across Systems and Phase Processes

| | Mixing | Zinc-Solubilizer | | | | Stabilization | | Clarifier | |
|---|---|---|---|---|---|---|---|---|---|
| | Phase | Enrichment Phase | | | | Phase | | Phase | Product |
| ZST | 8.66 | 5.65 | | 5.73 | | 6.12 | | 6.89 | 7.11 |
| 1.5 | | | | | | | | | |
| ZST | 8.66 | 7.662 | | 7.42 | | 7.84 | | 7.75 | 7.56 |
| 1.5+ | | | | | | | | | |
| ZST | 7.90 | 6.47 | 5.95 | | 6.39 | 6.99 | 7.05 | 7.46 | 7.56 |
| 2.0 | | | | | | | | | |
| FBR | | | | | | | | | |
| ZST | 8.40 | 6.78 | 6.40 | | 6.11 | 6.61 | 6.73 | 6.81 | 6.94 |
| 2.0 | | | | | | | | | |
| PBR | | | | | | | | | |

### Example 2: Structural Characterization and Description of Zinc Solubilizing Technology (ZST) sIP Systems and System Clarifier in ZST sIP Systems

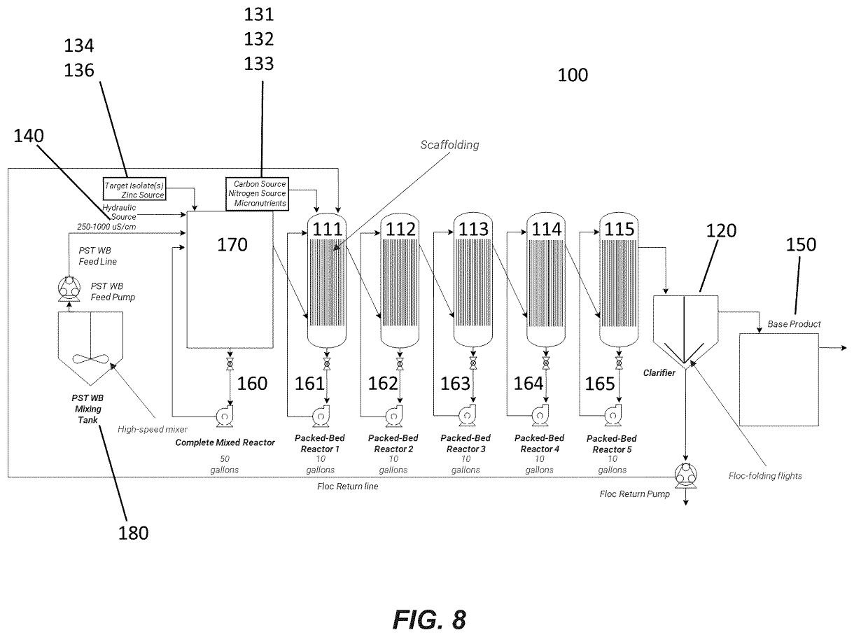

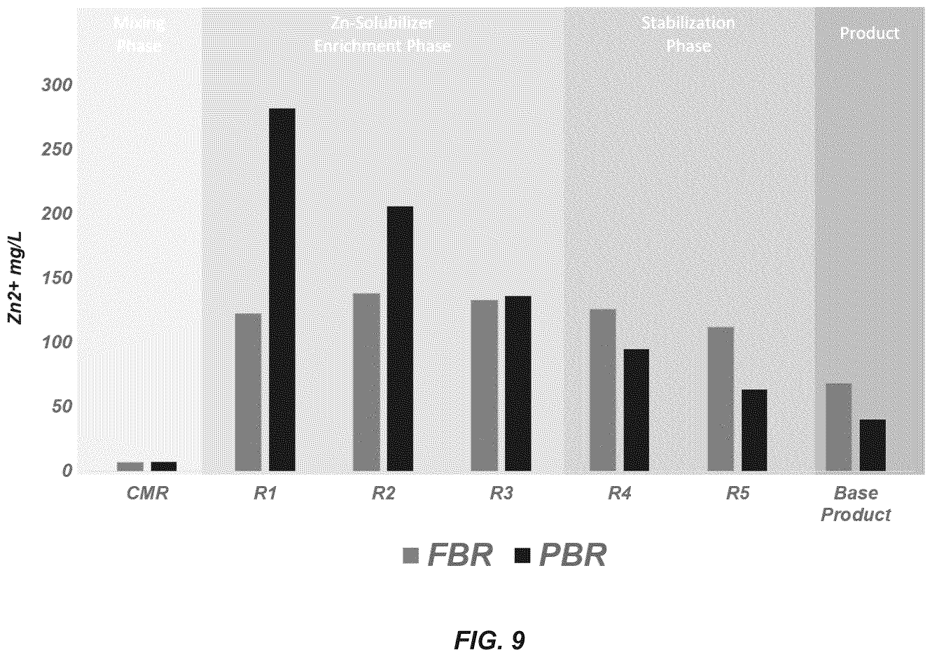

In this experiment, a serialized isolate production (sIP) system, referred to herein as ZST 1.5, was used to produce a biostimulant with an enriched population of zinc-solubilizing microbes as well as microbial digestion products that promote zinc solubilization when applied to plants and/or soil. The ZST 1.5 system included a series of four FBRs maintained at microaerobic conditions. Microaerobic/oxygen limited conditions may be defined as DO<2 mg/L. The ZST 1.5 system was characterized by an up-flow design as a continuous serialized system. The feed into the first reactor (R1), or referred as complete mixed reactor (CMR), of the ZST 1.5 system was water, zinc oxide, WB (1:1) from a phosphate solubilizing system, and a return feed of floc from a later reactor in the ZST-SIP system. Zinc was added in the form of ZnO (1-3 g per day) and in amounts that resulted in low soluble Zn2+ concentration within the first reactor (FIG. 9). When the ZST 1.5 system was initially established, an inoculum of microbial strain MS4666 was added in the first reactor. Without being bound by theory, it is thought the high concentration of ZnO in the first reactor (from all ZST systems) creates an environment that in the first reactor selectively enriches the microbial population for MS4666 and zinc-solubilizing microbes present in the PST WB, and that this enrichment is maintained throughout the ZST 1.5 system. After one inoculation of MS4666 into the system, an enriched population of MS4666 was maintained in the first FBR and throughout the ZST 1.5 system as the system continuously operated without the need for subsequent reinoculation. Initial inoculation of MS4666 was at a rate of 2×10⁸ cfu/ml in the first reactor of the system. Fluid from the top of the first reactor continuously flowed into the second reactor in a hydraulically balanced fashion. Glucose and ammonium sulfate were also added to the second reactor to maintain the concentrations in the second reactor at [5 g/day] and [2 g/day], respectively. Fluid from the top of the second reactor was continuously flowed into a third FBR in a hydraulically balanced fashion, and fluid form the top of the third reactor was continuously flowed into a fourth reactor in a hydraulically balanced fashion. Fluid from the top of the fourth reactor was flowed into a separation chamber, where solids were allowed to settle. The supernatant ZST-1.5 biostimulant product was collected, and a feed including the settled floc was returned to the first reactor. The fluid within each reactor of the ZST 1.5 system was circulated at a rate that maintained the rock phosphate particles in suspension and created microaerobic conditions (dissolved oxygen content of 0.08-2 mg/L). The ZST 1.5 product contained Zn-solubilizing microbes, microbe-associated metabolites, and the target isolate MS4666 (Bacillus safensis).

Two additional ZST-sIP systems-referred to herein as ZST 2.0 FBR (fluidized bed reactor) and ZST 2.0 PBR (packed bed reactor) were established using the product of the ZST 1.5 system to initiate the systems. The ZST 2.0 PBR reactors were packed with scaffolding to increase biofilm and surface area inside each reactor.

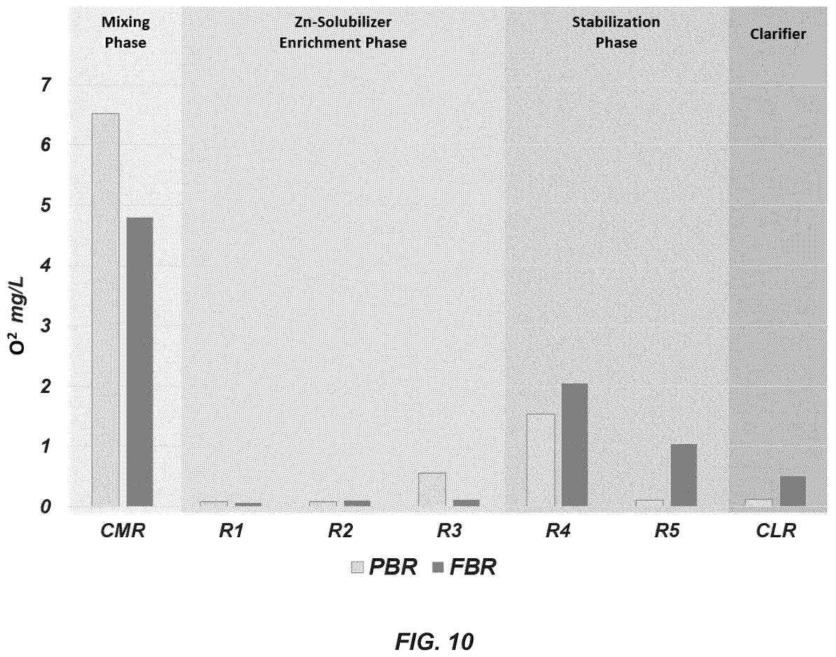

Progression through the reactors showed that ZST 2.0 PBR had reduced zinc solubilization (measured as zinc made soluble in mg/L) levels from CMR to the final product, whereas ZST 2.0 FBR demonstrated stable levels of zinc solubilization (FIG. 9). The ZST process was a microaerobic process. Both FBR and PBR systems showed similar dissolved oxygen measurements across the ZST process (FIG. 10). Dissolved oxygen levels for both systems across the four phases are shown in Table 12.

TABLE 12
Dissolved oxygen measurements (mg/L) for ZST 2.0 systems

| | CMR | R1 | R2 | R3 | R4 | R5 | CLR |
|---|---|---|---|---|---|---|---|
| ZST 2.0 PBR | 6.52 | 0.08 | 0.08 | 0.56 | 1.54 | 0.11 | 0.12 |
| ZST 2.0 FBR | 4.8 | 0.07 | 0.1 | 0.12 | 2.05 | 1.05 | 0.51 |

The clarifier in the ZST 2.0 systems retained the concentration levels of the isolate within a continuous mixed consortium process. The clarifier collected biosolids (i.e. floc and WB) generated in the system. Rotating flights in the clarifier folded the floc to ease floc return to the system and returned floc back to the start of the system of the zinc solubilization phase and reinoculated beneficial bacteria and target isolates. The clarification phase of the ZST process separated the supernatant from the floc. Floc returned from the clarifier back to the start of the zinc-solubilizer enrichment phase served as the seed of beneficial microbes and target isolate.

### Example 3: Plant Growth Promotion Capacity of MS4666 Isolate

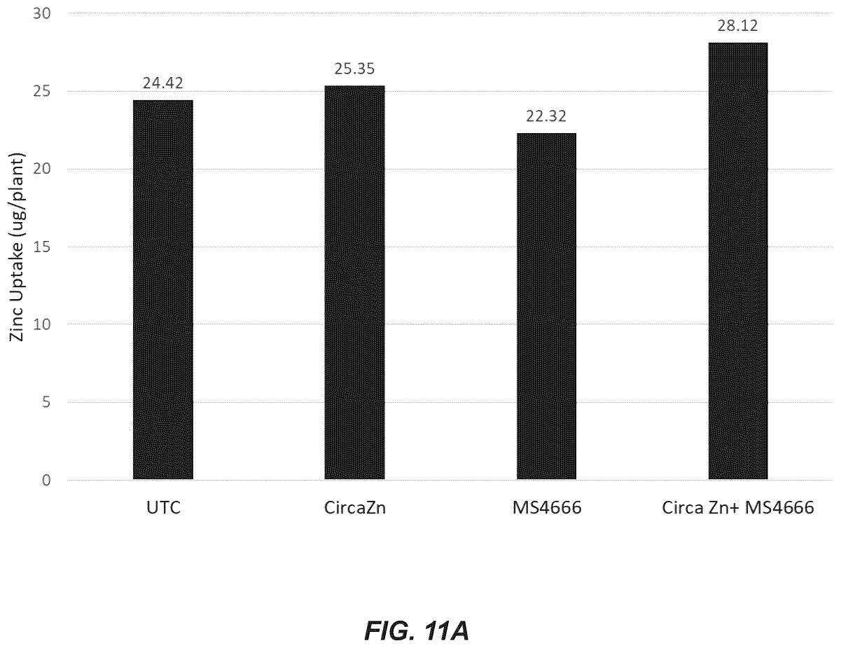

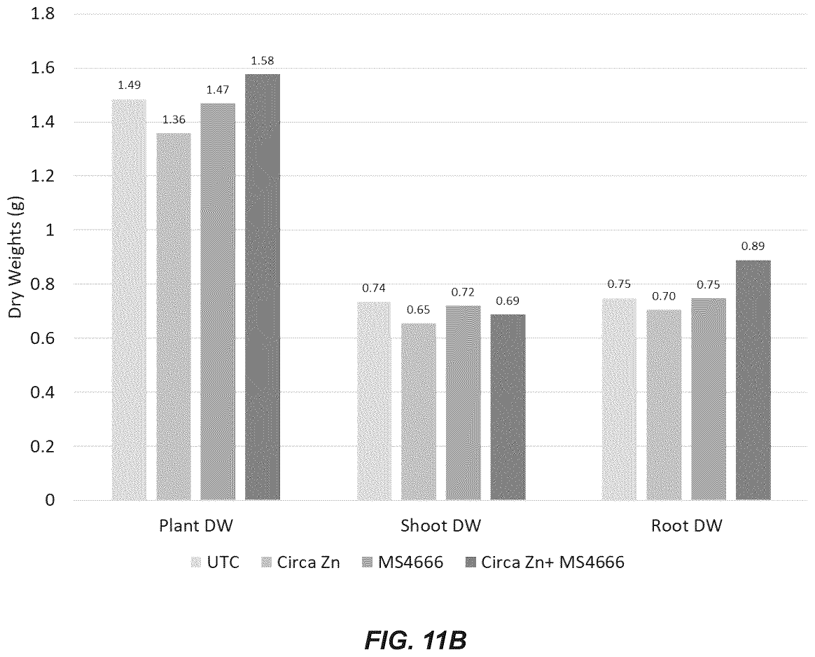

Plant dry weights and nutrients uptake levels were evaluated to assess plant growth promotion capacity of isolate MS4666. PGP was tested in navy beans following fertilizer coating on MAP (FIGS. 11A-11B). MAP prills were sprayed with treatments at the application rate and left to air dry overnight. The untreated control (UTC) was water, Circa™ Zn was applied at 160 oz/ton, MS4666 was applied at 64 oz/ton, and MS4666 with Circa™ Zn was applied at 64 oz/ton and 160 oz/ton respectively. The combination of MS4666 and Circa™ Zn showed the greatest levels of zinc uptake in navy beans compared to that of the other three treatment conditions (FIG. 11A). Total plant dry weights, shoot dry weights, and root dry weights were measured for navy bean plants. MS4666 with Circa™ Zn showed the greatest total plant dry weight, demonstrating superior PGP (FIG. 11B).

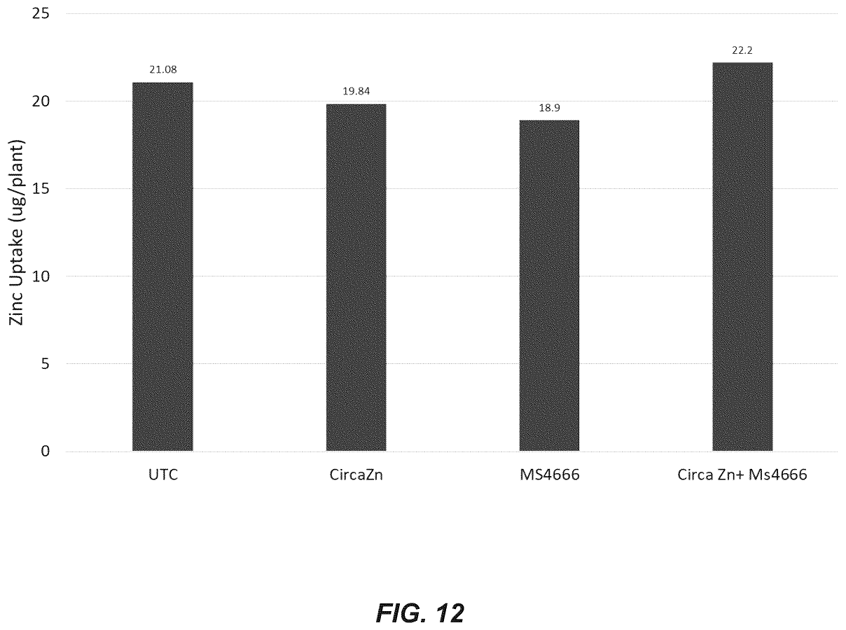

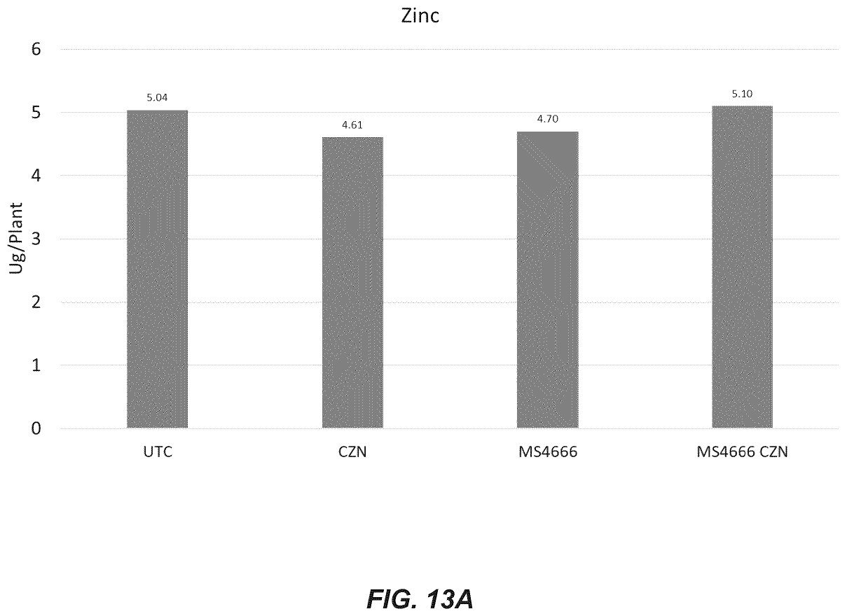

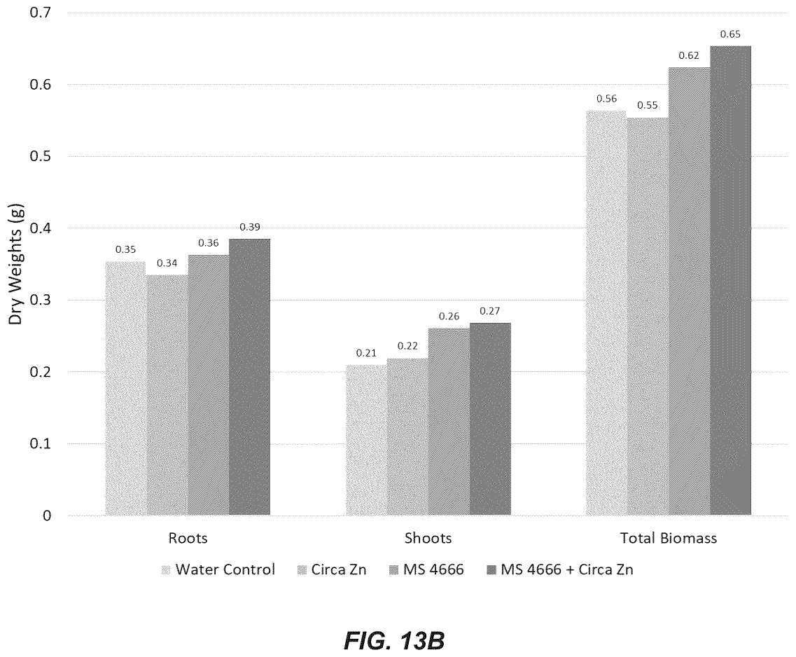

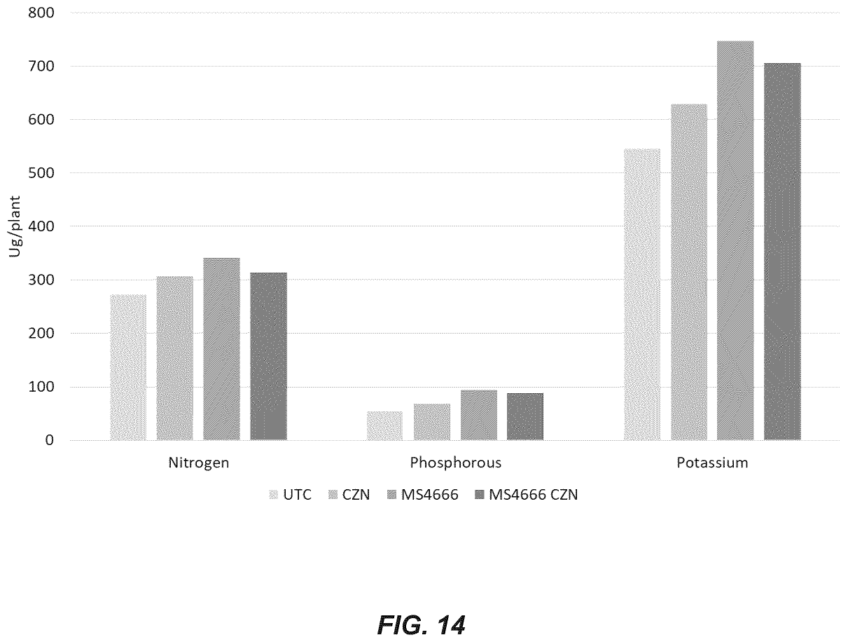

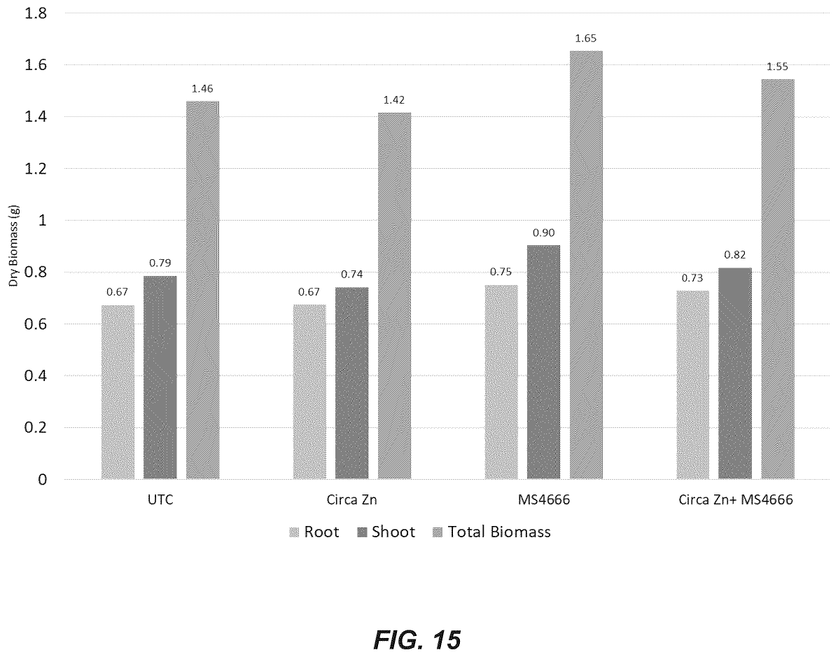

Similar protocols were followed to test PGP in corn. MAP was coated at 64 oz/ton with the following treatments: (UTC) was water, Circa™ Zn, MS4666 (at 1×10⁶ CFU/ml) was applied at 64 oz/ton, and MS4666 combined equally with Circa™ Zn. Three experiments were conducted. In the first experiment, the combination of MS4666 and Circa™ Zn showed the greatest levels of zinc uptake compared to that of the other three treatment conditions (FIG. 12). Application of MS4666 and Circa™ Zn together also resulted in the greatest root dry weight, shoot dry weight, and total biomass. For the second coated-fertilizer experiment in corn, the combination of MS4666 and Circa™ Zn again showed the greatest levels of zinc uptake compared to that of the other three treatment conditions (FIG. 13A). Application of MS4666 and Circa™ Zn together again resulted in the greatest root dry weight, shoot dry weight, and total biomass (FIG. 13B). In the second experiment, levels of macronutrients were also measured across the four treatment conditions. MS4666 alone and MS4666 with Circa™ Zn showed higher levels of nitrogen, phosphorous, and potassium compared to levels from Circa™ Zn and UTC (FIG. 14). In the third coated-fertilizer experiment, Circa™ Zn, both alone and in combination with MS4666, was applied at a rate of 160 oz/ton. MS4666 alone showed the greatest biomass compared to the other three treatments (FIG. 15). Over the three experiments, MS4666, both alone and applied with Circa™ Zn, demonstrated greater PGP capacity than that from UTC and Circa™ Zn alone.

### Example 4: Plant Growth Promotion Capacity of ZST Systems and MS4666 isolate

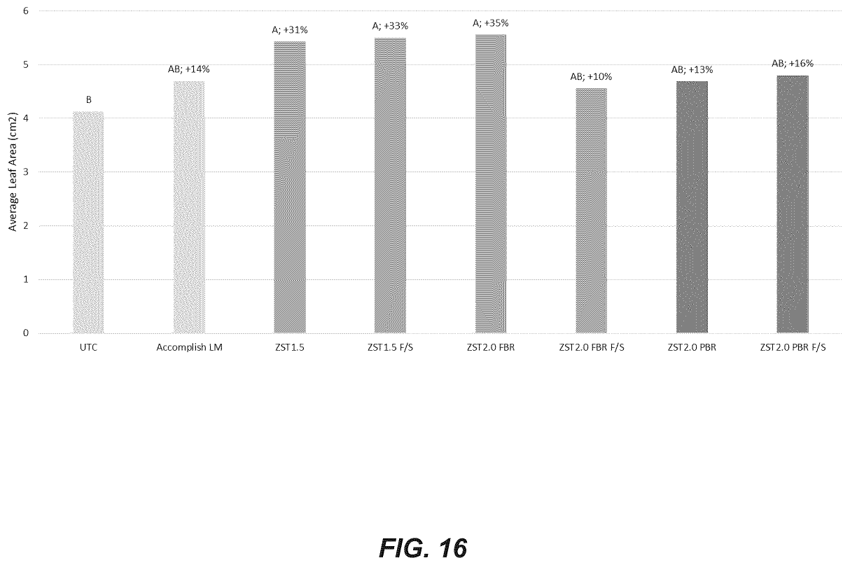

Plant growth promotion was tested in Arabidopsis for the three ZST systems as both intact and filter-sterilized (F/S; i.e., metabolite) solutions (FIG. 16). Arabidopsis thaliana (genotype Col-0) seedlings were germinated and grown for 7 days on ½ strength MS media (Murashige & Skoog Basal Medium with Vitamins, M519) plates in the growth chamber before transplanting into rockwool cubes for plant growth assays. Rockwool was used as an inert substrate for growing Arabidopsis in a hydroponic-like system. The rockwool cubes may be fitted into a 6-well tray and each treatment consisted of three replicate well trays. Eight 1 L ½ strength MS media (Murashige & Skoog Basal Medium with Vitamins, M519) solutions were made. The test substance was then added at the rate given below in Table 13, and the pH of the solution was raised to 5.7. Forty mL of each 1 L treatment solution was dispensed to each rockwool cube. Then, each wetted cube received one Arabidopsis seedling. Arabidopsis plants grew on an LED light cart for 12 days. Leaf area was determined using the ImageJ software package. All the data collected were put through JMP for ANOVA analysis (a=0.05). ZST solutions was applied at 0.05% v/v. Percentages above bars denote percentage of UTC. All ZST outputs showed superior PGP compared to UTC. ZST 1.5 metabolites and intact ZST 2.0 FBR showed the greatest levels of PGP in Arabidopsis. Treatments are shown in Table 13.

TABLE 13
Treatments for testing plates.

| | | |
|---|---|---|
| 1. | ½MS Media+ | UTC |
| 2. | ½MS Media+ | 0.8% Accomplish LM (Control) (v/v) (8 mL/L) |
| 3. | ½MS Media+ | 0.05% ZST 1.5 SPN (5 mL/L) |
| 4. | ½MS Media+ | 0.05% ZST 1.5 SPN F/S (5 mL/L) |
| 5. | ½MS Media+ | 0.05% ZST 2.0 FBR SPN (5 mL/L) |
| 6. | ½MS Media+ | 0.05% ZST 2.0 FBR SPN F/S (5 mL/L) |
| 7. | ½MS Media+ | 0.05% ZST 2.0 PBR SPN (5 mL/L) |
| 8. | ½MS Media+ | 0.05% ZST 2.0 PBR SPN F/S (5 mL/L) |

In a greenhouse study, MAP dry fertilizer was coated with MS4666 and Circa™ Zn to measure zinc nutrient uptake in corn. Corn was planted in a potting mix of isolite: Sunshine LC-1 (4:1) with one plant per pot and 12 replicate pots per treatment. One week after planting, corn was treated with the respective treatment and harvested five weeks following treatment. A list of treatments and application rates is in Table 14. A batch of 250g of coated fertilizer was made for each treatment. To the 250 g of fertilizer, a total delivery volume of 5 mls was used and the treatments were incorporated to the total 5 mLs based on the coated application rate. The data for all metrics were analyzed using JMP software (SAS Institute, Cary, NC, USA) using the Fit Model procedure and mean separation at alpha=0.1 level of significance.

TABLE 14
Characteristics of treatments in corn greenhouse study

| | Coating Rate (oz/ton) | |
|---|---|---|
| Treatment | Treatment | Circa ™ Zn |
| Water Control (UTC) | | |
| Circa ™ Zn | | 64 |
| MS4666 (1 × 10⁶ CFU/ml) | 64 | |
| MS4666 (1 × 10⁶ CFU/ml) + Circa ™ Zn | 64 | 64 |

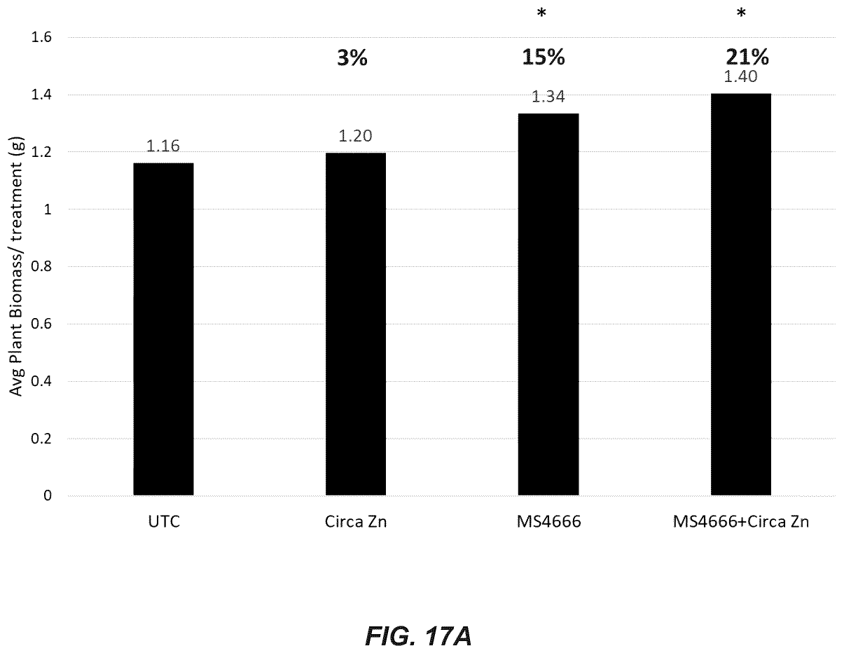

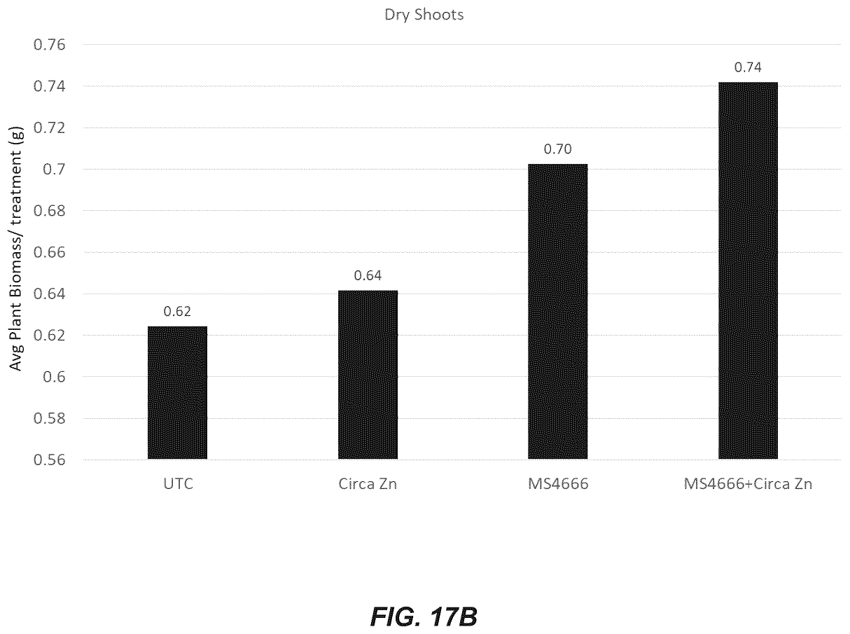

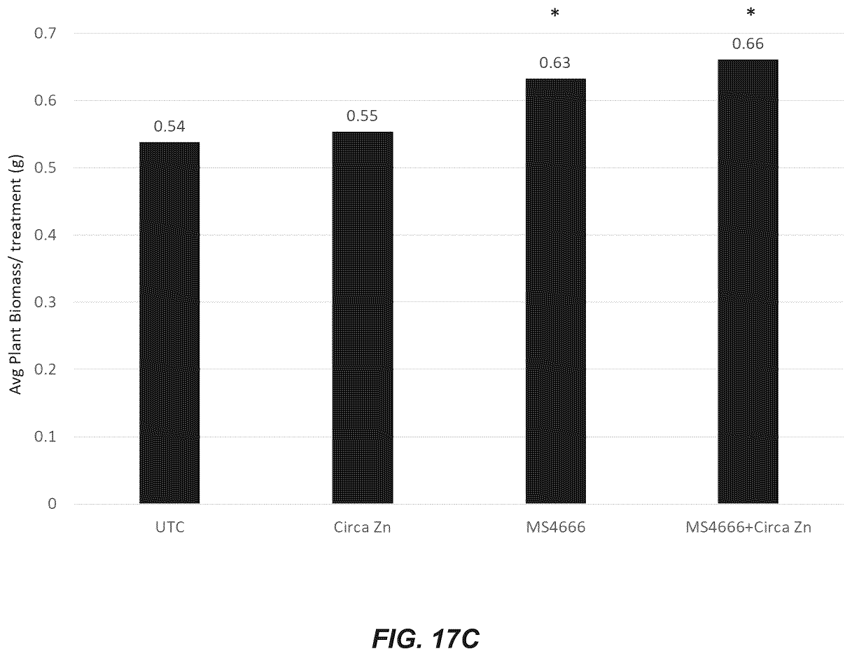

The target isolate, MS4666, both alone and in combination with Circa™ Zn, produced significantly greater total dry biomass than UTC (FIG. 17A). Both conditions showed greater total biomass than other solubilizing technology treatments. Percentages above the bars denote percent change compared to UTC. MS4666 alone, and in combination with Circa™ Zn, also showed the greatest dry biomass for corn shoots (FIG. 17B) and corn roots (FIG. 17C) compared to UTC and other treatments. Addition of Circa™ Zn to ZST SPN or target isolate showed improved growth compared to the ZST SPN or target isolate alone.

A second corn greenhouse study was conducted evaluating the efficacy of MAP fertilizer coated with ZST 1.5 product, PST WB, and MS4666, either alone or in combination with Circa™ Zn, on zinc uptake. Plants were planted in round 15 cm diameter pots and fertilized with MAP at 25 1b N/acre and 175 1b P₂O₅/acre and MOP (KCl) at 50 lb K₂O/acre. A list of treatments and coating rates is in Table 15.

TABLE 15
Characteristics of treatments

| | Coating Rate (oz/ton) | |
|---|---|---|
| Treatment | Treatment | Circa ™ Zn |
| UTC (RO Water) | 64 | |
| Circa ™ Zn | | 64 |
| PST WB 1:1 | 64 | |
| PST WB + Circa ™ Zn | 64 | 64 |
| ZST 1.5 SPN | 64 | |
| ZST 1.5 SPN+ Circa ™ Zn | 64 | 64 |
| MS4666 (1 × 10⁶ CFU/ml) | 64 | |
| MS4666 (1 × 10⁶ CFU/ml) + Circa ™ Zn | 64 | 64 |

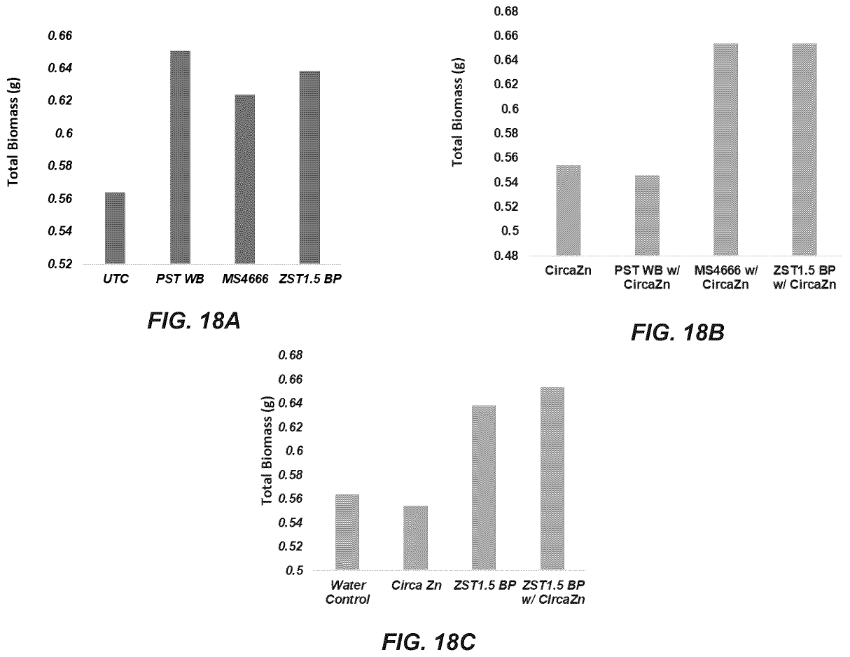

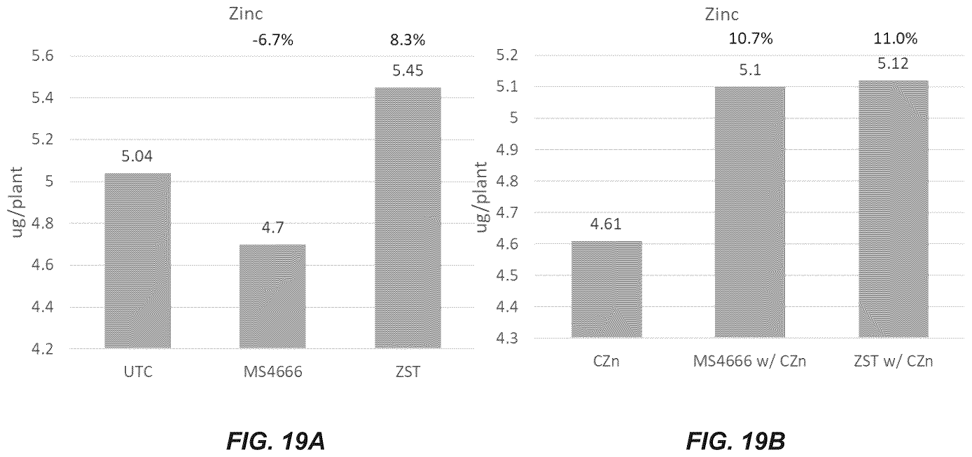

In measuring PGP between the components for ZST 1.5, MS4666, PST WB, and ZST 1.5 base product (BP) showed greater total biomass compared to UTC (FIG. 18A). Both MS4666 with Circa™ Zn and ZST 1.5 SPN with Circa™ Zn showed superior total biomass compared to that from Circa™ Zn alone and PST WB with Circa™ Zn (FIG. 18B). Plants treated with ZST 1.5 SPN or ZST in combination with Circa™ Zn also showed greater total biomass compared to that from UTC and Circa™ Zn alone (FIG. 18C). Components of ZST and ZST 1.5 SPN demonstrated growth promotion in corn. ZST output provided better zinc uptake than UTC and MS4666 alone (FIG. 19A). Addition of Circa™ Zn to MS4666, and ZST led to greater zinc uptake than that from Circa™ Zn alone (FIG. 19B).

ZST performance, for zinc nutrient uptake and plant growth promotion alone and in combination with Circa™ Zn, was evaluated in a long-term greenhouse study where corn was allowed to grow to maturity. The study was a Latin square design with 10 replicate pots per treatment. Corn was planted into 2-gallon buckets containing a growing medium mixture of 9:1 isolite: Sunshine LC1 Mix. Treatments were coated on to potash at a rate of 64 oz/ton and application of the coated fertilizer at 50 lb N/acre. Treatments with ZST technologies and Circa™ Zn were combined at a 7:1 (Circa™ Zn: ZST Tech) ratio. Treatments and applications rates can be found in Table 16.

TABLE 16
Treatments and Application Rates for Long-Term Greenhouse Study

| Treatment | Circa ™ | ZST Tech | Total |
|---|---|---|---|
| | Zn (oz/ton) | (oz/ton) | Application (oz/ton) |
| UTC | | | |
| Circa ™ Zn (CZN) | 64 | | 64 |
| ZST 1.5 | | 8 | 8 |
| ZST 1.5 with CZN | 56 | 8 | 64 |
| ZST 2.0 FBR | | 8 | 8 |
| ZST 2.0 FBR with CZN | 56 | 8 | 64 |
| ZST 2.0 PBR | | 8 | 8 |
| ZST PBR with CZN | 56 | 8 | 64 |

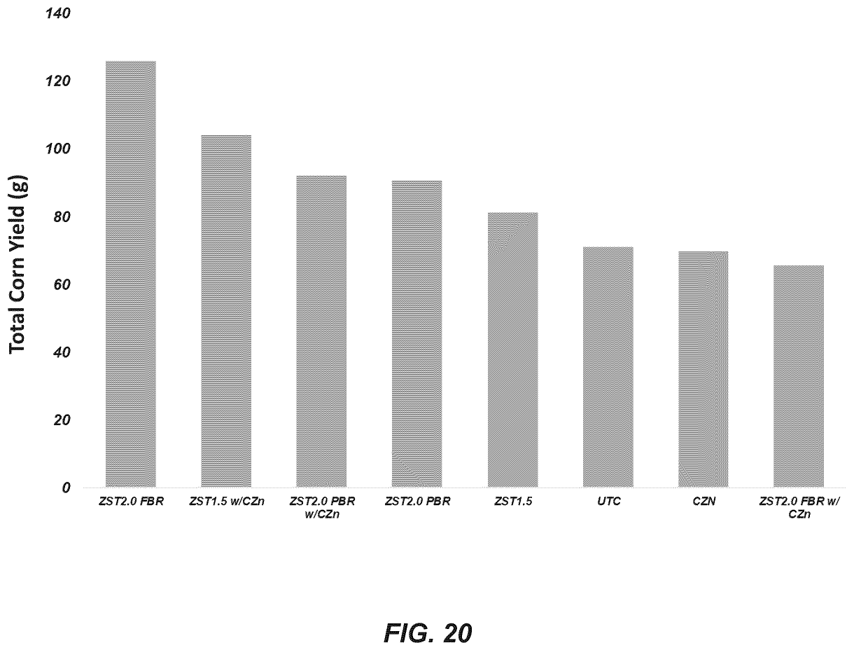

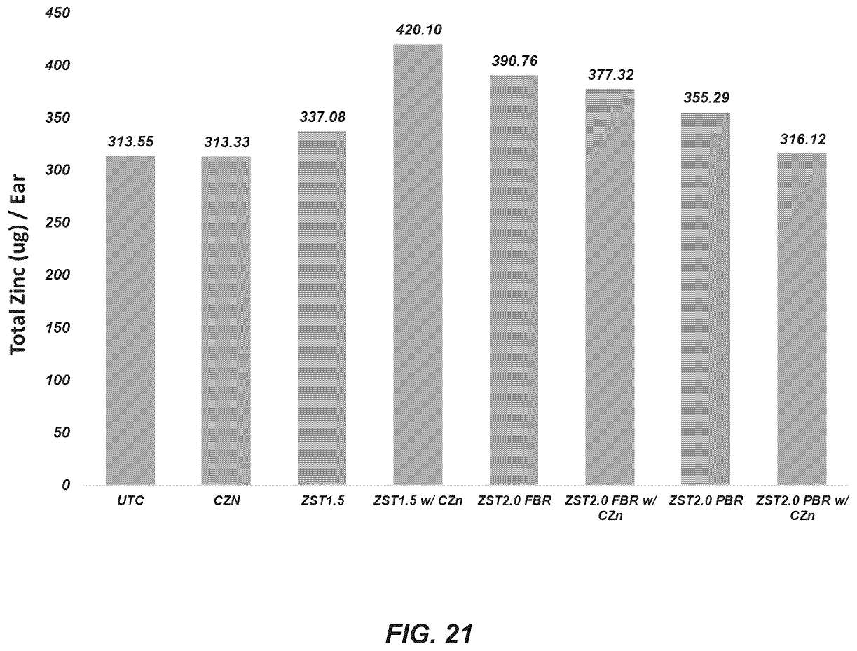

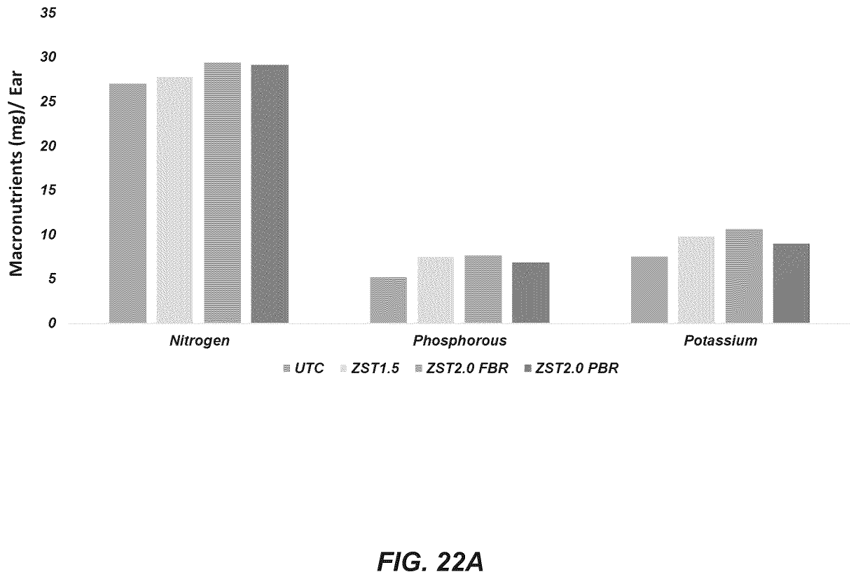

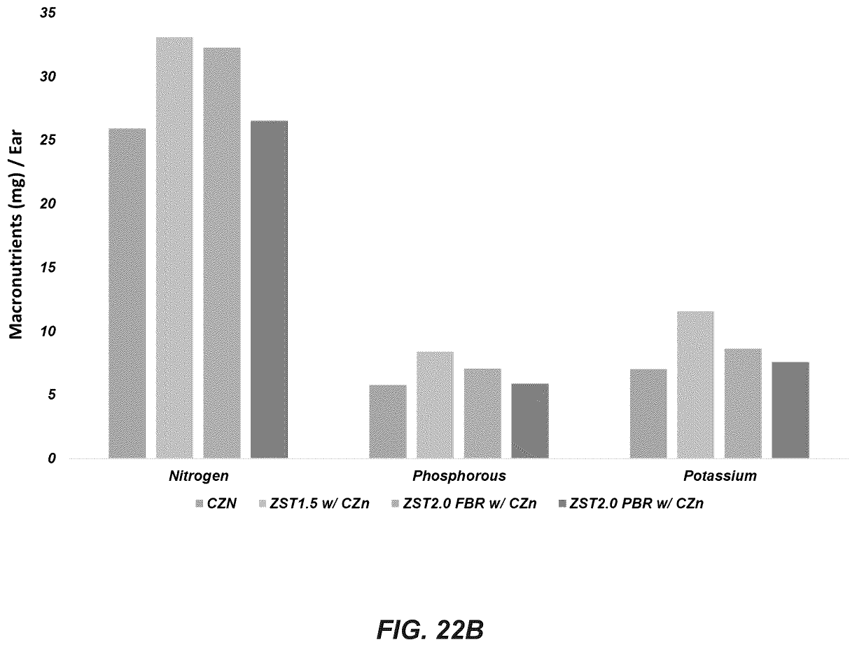

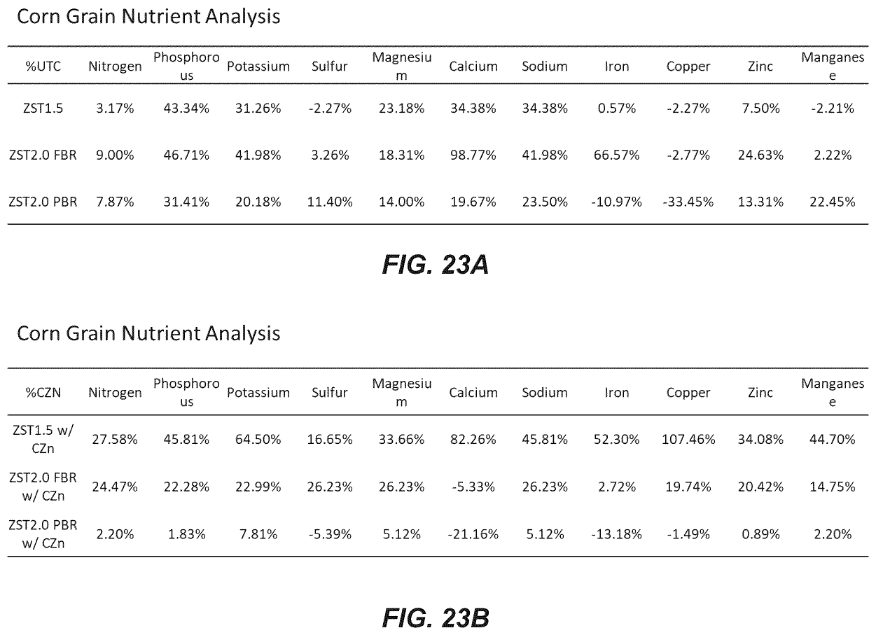

ZST 2.0 FBR product showed the greatest PGP, measured as total corn yield, compared to UTC, CZN, and other ZST treatments (FIG. 20). For ZST 1.5 and ZST 2.0 PBR, addition of CZN improved total corn yield compared to that from the two systems alone. Treatment with ZST system outputs alone and ZST system outputs with CZN increased the total zinc per ear of corn compared to that of UTC or CZN alone (FIG. 21). ZST 1.5 SPN with CZN showed the greatest zinc per ear. Ears of corn were tested for levels of nitrogen, phosphorous, and potassium following treatment. Each ZST system alone showed greater total macronutrients per ear compared to the total macronutrients from UTC (FIG. 22A). ZST 2.0 FBR showed the highest levels of each macronutrient. ZST systems with CZN were also tested for their effects on macronutrient levels in corn. ZST systems with CZN led to improved macronutrient levels per ear compared to that from CZN alone, and ZST 1.5 with CZN showed the greatest levels of each macronutrient (FIG. 22B). A summary of macronutrient and micronutrient content from ZST treatments alone and ZST treatments with CZN can be found in FIG. 23A and FIG. 23B respectively.

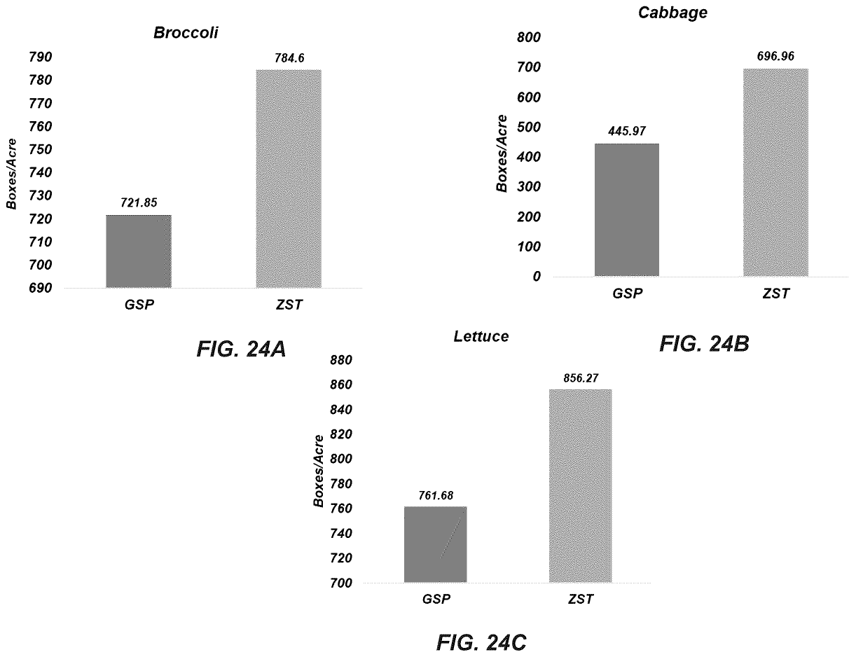

In field studies in Yuma Arizona, the ZST 2.0 FBR system product performance was evaluated for plant growth promotion across three difference crops (broccoli, cabbage, and lettuce). The studies were set up in a 7′×25′ non-randomized plot with 6 replicates per treatment. The fertility program for these tests was 350 lbs of MAP and two different applications of 20 gallons of CAN17. For testing, ZST was applied at 2 qt/acre in a fertigation system. In all three crop tests, the ZST system led to a greater number of harvested boxes per acre than Grower Standard Practice (GSP), demonstrating its plant growth promotion capacity. In broccoli, application of ZST led to 784.6 boxes/acre (FIG. 24A). In cabbage, application of ZST led to 696.96 boxes/acre (FIG. 24B). In lettuce, application of ZST led to 856.27 boxes/acre (FIG. 24C).

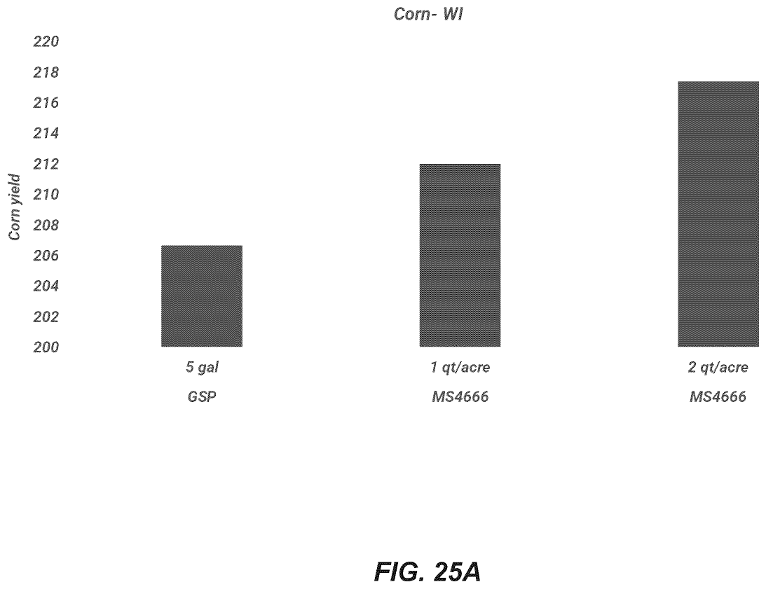

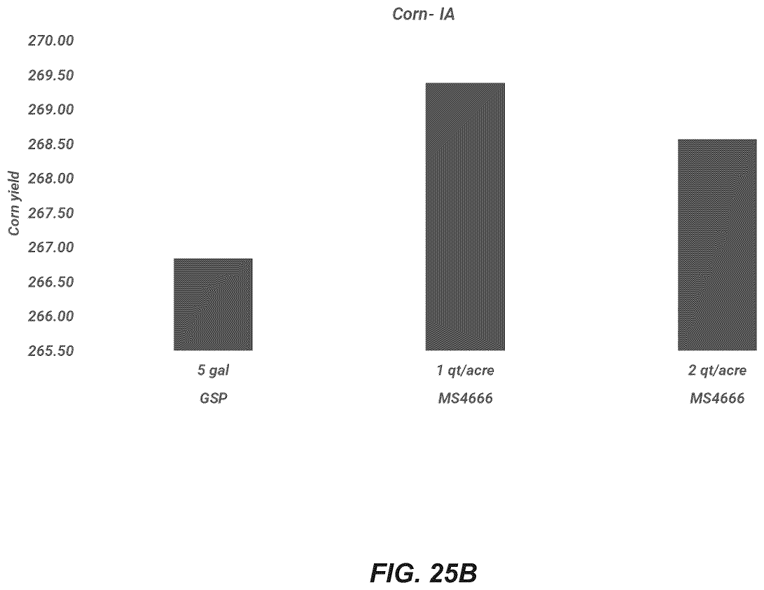

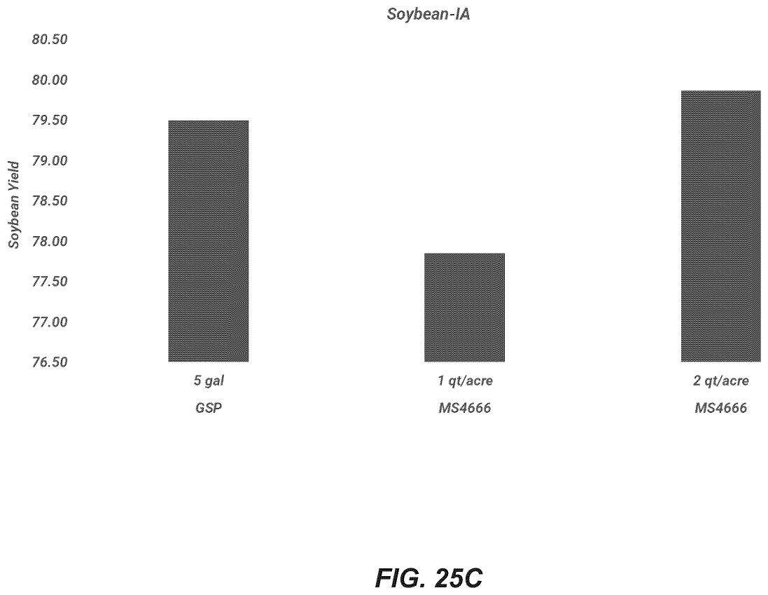

In field studies in Wisconsin and Iowa, target isolate MS4666 was evaluated for plant growth promotion across corn and soybean. The grower's standard practice (GSP) was 5-gallon application of 10-34-0 in furrow for corn and an application of 5-gallons of Mar. 18, 2018 in furrow for the Iowa soybean test. The application rate of MS4666 was 1 and 2 quarts per acre in furrow application. The application of MS4666 led to higher yield in corn compared to that from GSP (FIGS. 25A-25B). The application of MS4666 at 2 qt/acre led to a greater soybean yield compared to that from GSP (FIG. 25C).

### Example 5: Synergy of Target Isolate in the ZST 1.0 System

For this experiment, ZST 1.0 batch systems were carried out for 11 days to understand the fermentation process with different loading inputs.

An experiment was designed to evaluate the effects of MS4666 in a ZST-1.0 batch system to improve efficacy measured in the form of zinc solubilization capacity. Table 17 displays the treatments used in each batch reactor. The hydraulic source for the ZST both batches (Batch-A and Batch-B) systems was water at an electrical conductivity ranging from 250-1000 microsiemens/centimeter (μS/cm). In both Batch reactor systems (Batch-A and Batch-B), zinc oxide was added into the batch reactor at a concentration of 0.1% w/v, as well as PST floc of the phosphate solubilizing technology at a concentration of 1.0% v/v. Glucose and ammonium sulfate were added into the reactor to maintain a concentration range of 1.0% w/v and a concentration 0.1% w/v, respectively. Potassium chloride (KCl), magnesium sulfate (MgSO₄), and dipotassium phosphate (K₂HPO₄) were added to both batch reactor at a concentration of 0.02% w/v, 0.02% w/v, and 0.1% w/v, respectively. Batch-B was inoculated with target isolate MS4666 at a rate of 1.00E+06 cfu/ml. MS4666 was grown in microbial culture media, Tryptic Soy Broth (TSB, Difco) for this test. The test ran for a total of 7 days and samples were collected at the end of the experiment. The zinc made soluble during the process was quantified by Inductively Couples Plasma (ICP) Atomic Emission Spectrometer.

TABLE 17
List of treatment conditions

| | | Aqueous | | Target Isolate |
|---|---|---|---|---|
| | | Feedstock | | Application |
| | Aqueous | application | Target | Rate |
| Batch Reactor | Feedstock | rate (v/v) | Isolate | (cfu/ml) |
| Batch-A | PST-Floc | 1% | | |
| Batch-B | PST-Floc | 1% | MS4666 | 1.00E+06 |

Along with the batch system solutions, the ZST microbial components, MS4666 and the PST floc, were used in zinc solubilization capacity test, similar to the batch systems. Test solutions were added to media Zn-solubilizing medium (Saravanan 2003, the same media components and concentrations used in the batch system experiment) using ZnO as the insoluble source of Zn in a 50 mL conical tubes. For the MS4666 culture, an overnight grown culture of MS4666 in Tryptic Soy Broth (TSB, Difco) was used and test tubes were inoculated at 1.00E+06 cfu/ml. The tubes were incubated at 30° C. at 200 rpm for 7 days and Zn2+ made soluble was measured using the ICP.

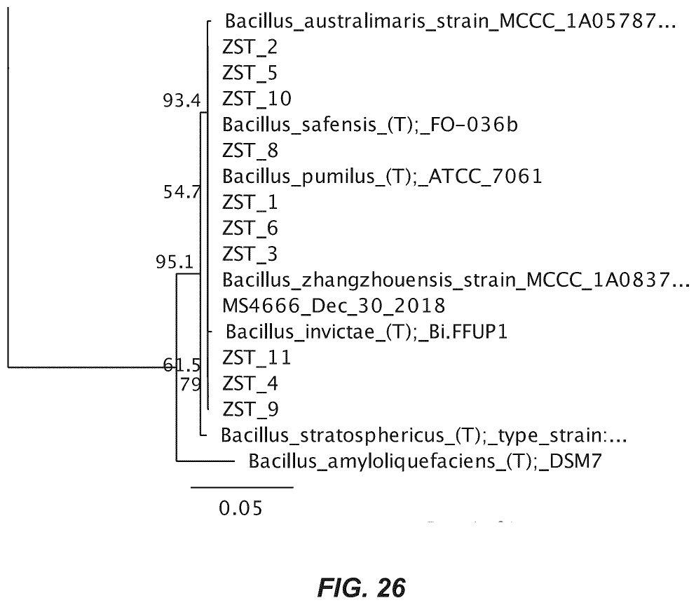

Solutions from each batch system were analyzed for both unheated and heated (pasteurized) samples to determine the abundance of total bacteria and spore-forming bacteria in the solution, respectively and to identify target isolate MS4666. The microbial nutrient media used was ¼-strenght TSA (Tryptic soy agar, BD, Difco #236920) media. The plates were incubated at 30° C. for up to seven days and then total colony forming units (CFUs) were determined. After seven days, colonies that match the morphology of MS4666 were verified by DNA sequencing of the 16s rRNA gene. FIG. 26 details the phylogenetic tree based on the sequencing alignment. Table 18 shows the isolate and isolation concentration.

TABLE 18
Listing of bacterial colonies selected for 16s rRNA sequencing

| Isolate Code | Batch System | Solution | Concentration |
|---|---|---|---|
| ZST_1 | Batch-B | Intact | 1e⁷ |
| ZST_2 | Batch-B | Intact | 1e⁷ |
| ZST_3 | Batch-B | Intact | 1e⁷ |
| ZST_4 | Batch-B | Pasteurized | 1e⁴ |
| ZST_5 | Batch-B | Pasteurized | 1e⁴ |
| ZST_6 | Batch-B | Pasteurized | 1e⁴ |
| ZST_8 | Batch-B | Intact | 1e⁶ |
| ZST_9 | Batch-B | Intact | 1e⁶ |
| ZST_10 | Batch-B | Pasteurized | 1e⁴ |
| ZST_11 | Batch-B | Pasteurized | 1e⁴ |

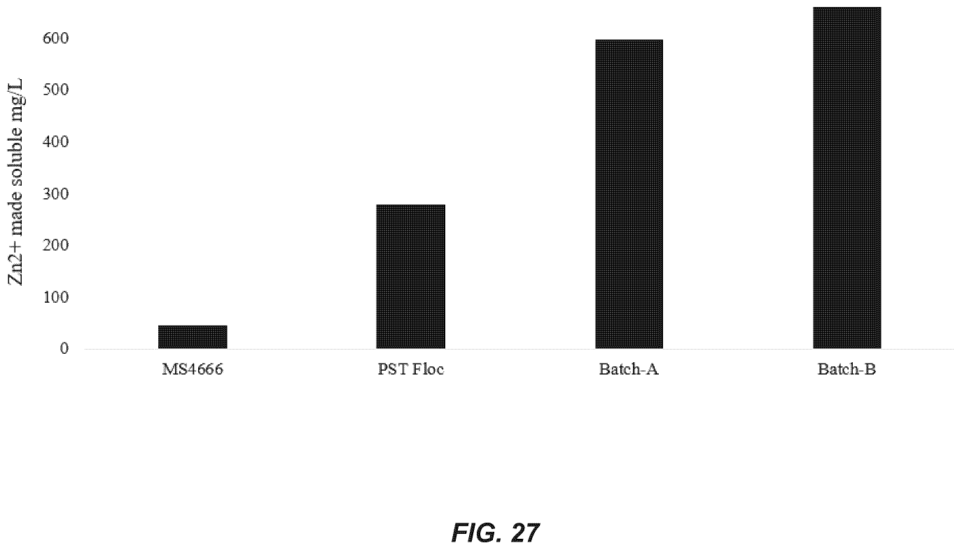

The results from the test showed the zinc solubilization potential of each individual microbial component (MS4666 and PST WB) as well as the batch systems (FIG. 27). The amount of zinc made soluble from the batch systems was far greater than the MS4666 or PST Floc treatments along and combined. The amount of zinc that was made soluble during the fermentation run was greater in Batch-B system that was inoculated with the target isolate MS4666. MS4666 was able to improve the solubilization of ZnO in the system by over 10% more than a Batch-A.

Sequencing analysis confirmed that MS4666 was detectable at higher concentrations (1.00E+07 cfu/ml) than original inoculation. This showed the potential for growing and enriching for a target isolate within a mixed consortium and improving the functionality of zinc solubilization

### Example 6: Functional Enrichment of Zn-Solubilizers in ZST System and Base Product

In this example, working solution from reactor in the process series along with the base product of the process was evaluated using culture-dependent methods for enumerating zinc solubilizers and an in vitro test to measure the solubilization capacity of ZnO from the solutions.

Standard dilution plating of the samples was completed to determine overall total bacteria and zinc-solubilizing bacteria abundance. Two replicate subsamples of the samples were passed through a series of 10-fold dilutions and 0.1 ml plated on standard microbial nutrient media. The microbial nutrient media used was ¼-strenght TSA (Tryptic soy agar, BD, Difco #236920) media and selective zinc-solubilizing media used in Saravanan 2003 was used. The plates were incubated at 30° C. for up to seven days and then zinc solubilizers were determined based on the solubilization zone of clearing created on the plate, signifying solubilization of ZnO. Samples were also analyzed for Zn2+in solution from each reactor and in the base product by Inductively Couples Plasma (ICP) Atomic Emission Spectrometer.

An in-vitro zinc solubilizing efficiency test was used to quantify the zinc solubilization potential of treatment solutions using insoluble zinc in the form of ZnO. A 3% v/v test solution was added to 5 ml media Zn-solubilizing medium using ZnO as the insoluble source of Zn in a 50 mL conical tubes. The tubes were incubated at 30° C. at 200 rpm for 5 days and Zn2+ made soluble was measured. Free form Zn2+ made soluble was quantified by Inductively Couples Plasma (ICP) Atomic Emission Spectrometer. In assessing the difference between the solutions from reactor 1 and the base product from the ZST-1.5 system, a two-tailed t-test was conducted. (a=0.05, p<0.0001).

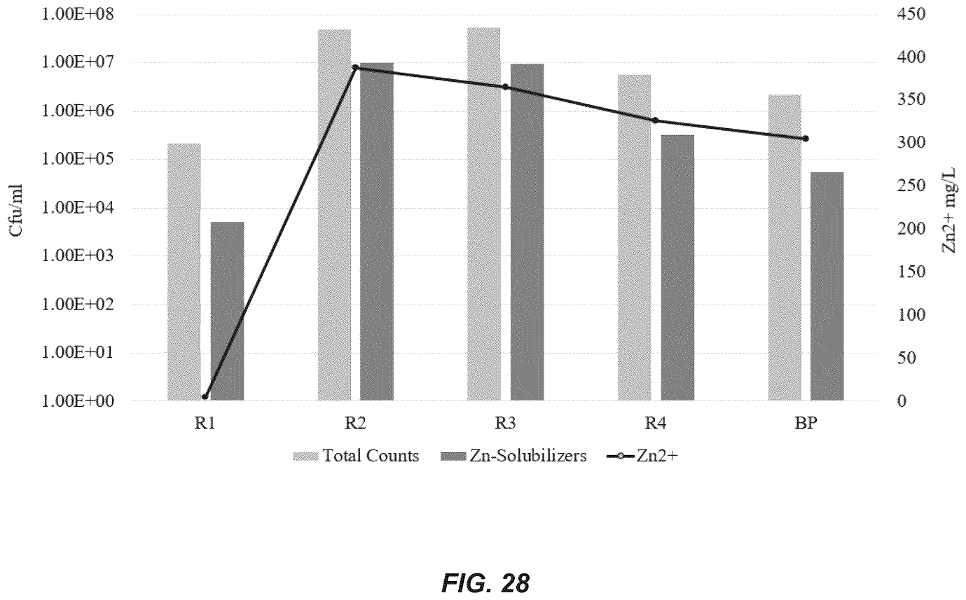

The results showed an enrichment of Zn-solubilizers once the fermentation phase starts in reactor 2 (R2). There was more than a 1000× increase change in the concentration of Zn-solubilizers from R1 to R2 and consistent through the fermentation phase of the process (FIG. 28). The proportion of Zn solubilizers relative to total bacteria also increased. The amount of Zn2+in solution increased as well during the fermentation phase of the process where the concentration of zinc-solubilizers was highest in the system. The base product had a 10× increase in Zn-solubilizers from the start of the process (R1). The results demonstrated the ZST process enriched for the targeted functionality by increased the concentration of Zn2+, total Zn-solubilizers, and proportion of Zn solubilizers to total bacteria through the process, with a base product containing more Zn-solubilizers than the starting point.

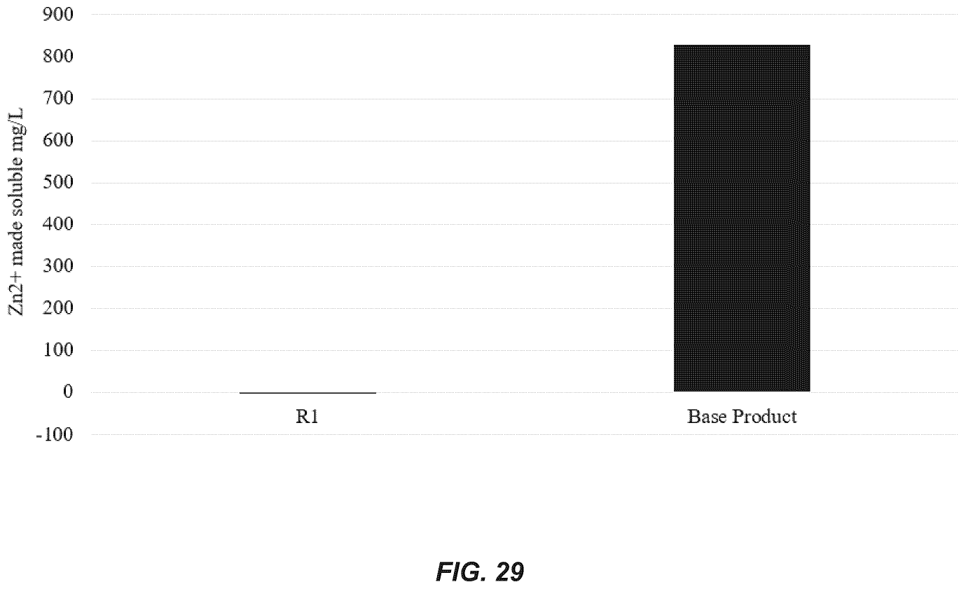

The results demonstrated the enrichment of functional capacity of the ZST process. The base product generated through the ZST process had greater zinc solubilization capacity than the working solution from the start of the process (FIG. 29). The results showed statistically significant increase of zinc solubilization by the base product in comparison to RI working solution. Both results together demonstrated that a functional enrichment of zinc solubilization through both an increase in Zn-solubilizers and Zn-solubilization capacity in vitro.

### Example 7: Effects of MS4666 on functionality of ZST system and in Base Product

In this example, working solution from each reactor in the process series along with the base product of the process was evaluated using in vitro test to measure the solubilization capacity of ZnO from the solutions. Samples from the ZST-2.0 FBR system, which has the target isolate, MS4666, as well as from ZST-2.5, which does not have a target isolate were collected.

The ZST 2.5 system was also a fluidized bed-based system that utilized PST WB at a 1:1 floc: supernatant ratio as the microbial inoculum. The system was based on the ZST 2.0 FBR system design (see Example 1). The orientation of the hydraulic re-circulation within each reactor was down-flow. The system included a fifth reactor at the end of the series before the clarifier as well as a complete mix reactor (CMR) at the front end of the system acting as the Reactor 1 of ZST1.5. The distribution of the working volume of the system was equally dispersed throughout the system. The system utilized PwST WB at a 1:1 floc: supernatant ratio as the microbial inoculum. Floc-flights were incorporated in the clarifier to fold the floc, to aid in the recirculation back upstream of the process (Reactor 1). In the ZST-2.5 system, zinc oxide was added into the CMR to maintain a concentration range of 0.05-0.5% w/v, based on the span of the hydraulic retention time of the system. The total volume of working fluid within all the reactors and clarifiers of the ZST-2.5 system was maintained at 115 gallons. The hydraulic rate of flow through the ZST-2.5 system was 14 days. In some embodiments of the ZST-2.5 system, the hydraulic rate of flow through the digestion system may be 21.59 mL/min to maintain a retention time of 14 days and can be varied accordingly for a retention time from 7 to 21 days depending on production volume requirements. Because of this, the amount of product produced per day can range from 5.48 to 16.43 gallons. The amount of product produced per week can range from 38.34 to 115.01 gallons. The ZST-2.5 system resulted in a 4.6× total system volume scale up from the ZST 1.5 system. The scale up of ZST1.5 system to the ZST-2.5 system used retention time as the constant variable to determine scale factors. In the ZST-2.5 system, zinc oxide gets added into the CMR to maintain a concentration range of 0.05-0.5% w/v, based on the span of the hydraulic retention time of the system. A concentration range of 0.5-8% v/v of Whole Broth of a Phosphate Solubilization Technology, PST WB (in a ratio of 1:1 floc: supernatant (SPN)) was also added to the CMR on the span of the hydraulic retention time of the system. Glucose and ammonium sulfate are added into Reactor 1 to maintain a concentration range of 0.5-3.0% w/v and 0.05-0.2% w/v, respectively, based on the span of the hydraulic retention time of the system. Potassium chloride (KCl), magnesium sulfate (MgSO₄), and dipotassium phosphate (K₂HPO₄) were added into Reactor 1 at a concentration range of 0.01-0.05% w/v, 0.01-0.05% w/v, and 0.05-0.2% w/v, respectively, based on the span of the hydraulic retention time of the system. Fluid from the top of the Reactor 1 was continuously flowed into Reactor 2 in a hydraulically balanced fashion. Fluid from the top of the Reactor 2 was continuously flowed into Reactor 3, and from Reactor 3 to 4, and from 4 to 5, in a hydraulically balanced fashion. In some cases, fluid from the top of a fifth reactor was flowed into a separation chamber (clarifier), where solids were allowed to settle. The hydraulic rate was 21.59 mL/min. The supernatant (base product) from the clarifier was continuously collected, and a portion of the floc at the bottom of the clarifier was returned to Reactor 1 at a rate of 2% v/v of the system total volume. In e, the dissolved oxygen in the CMR had a range of 5.00-9.00 mg/L with an average of 7.88 as it is highly mixed reactor. Following the start of the fermentation Reactor 1 range 0.05-1.2 mg/L with an average of 0.23 mg/L, Reactor 2, the dissolved oxygen range was 0.05-1.00 mg/L with an average of 0.18 mg/L, in Reactor 3 was 0.05-1.00 mg/L with an average of 0.14 mg/L, Reactor 4 was 0.10-2.00 mg/L with an average of 0.24 mg/L and/or in Reactor 5 was 0.05-4.50 mg/L with an average of 0.33 mg/L. The clarifier had a dissolved oxygen range was 0.05-1.20 mg/L with an average of 0.16 mg/L. In the ZST-2.5 system, pH in the CMR range 7.20-9.50 with an average pH of 8.76. Following the start of the fermentation in Reactor 1, the pH range was 4.00-9.00 with an average pH of 6.55, in Reactor 2 was 4.00-8.00 with an average pH of 6.19, in Reactor 3 was 4.00-8.00 with an average pH of 6.13, in Reactor 4 was 5.00-8.00 with an average of 6.14, and/or in Reactor 5 was 5.50-8.00 with an average of 6.28. The clarifier has a pH range of 5.00-7.00 with an average pH of 6.63. The base product has a pH range of 6.0-8.00 with an average pH of 6.73.

Free form Zn2+in the working solution of the reactors from each system was quantified by Inductively Couples Plasma (ICP) Atomic Emission Spectrometer.

Following the in-process characterization of the ZST systems, the functional capacity (Zn-solubilization) of the base product from both systems was measured. An in-vitro zinc solubilizing efficiency test (ZET) was used to quantify the zinc solubilization potential of treatment solutions using insoluble zinc in the form of ZnO. A 3% v/v test solution was added to media Zn-solubilizing medium (Saravanan 2003) using ZnO as the insoluble source of Zn. The inoculated media was incubated at 30° C. at 200 rpm for five days and Zn2+ made soluble was measured. Free form Zn2+ made soluble was quantified by Inductively Couples Plasma (ICP) Atomic Emission Spectrometer.

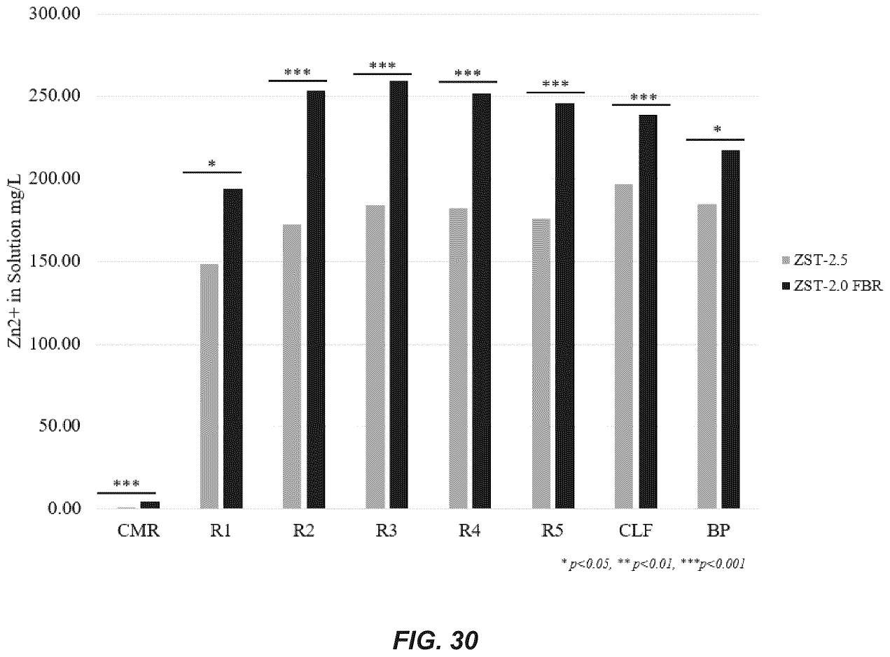

In assessing the difference between the solutions from each reactor and base product from either the ZST-2.0 FBR or the ZST-2.5, a two-tailed t-test was conducted. The results showed statistically significant differences for each solution in the reactor series as well as the base product (CMR p=0.0010, R1 p=0.0450, R2 p<0.0001, R3 p<0.0001, R4 p<0.0001, R5 p<0.0001, CLF p=0.0006, BP p=0.0174). The results suggested an improved functionality of zinc solubilization within the process system when MS4666 is incorporated within the continuous process (ZST-2.0 FBR) in comparison to a process that does not have MS4666 (ZST-2.5) (FIG. 30). The increase in Zn2+ concentration in the system may have improved the enrichment Zn-solubilizers.

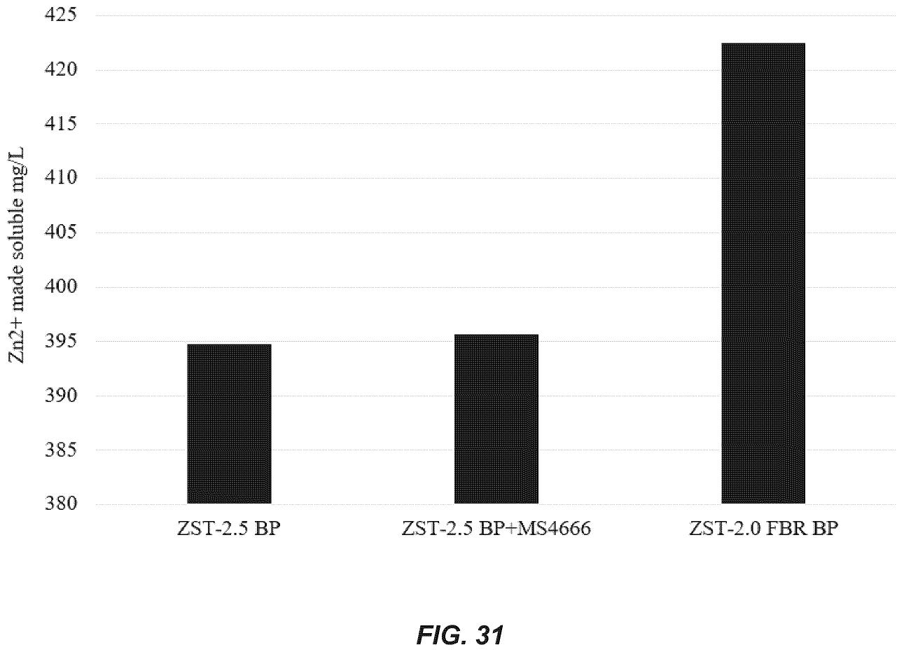

The results from the zinc solubilization efficiency test showed that the base technology, ZST-2.5 (with no isolate), and ZST-2.5 with MS4666 added to the base product, had similar solubilization capacities. However, the solubilization of ZnO by the ZST-2.0 FBR (has MS4666 in the system), shows a higher quantity of ZnO solubilized by up to 7% (FIG. 31). Results showed a potential benefit of having MS4666 in the system in increase the zinc solubilization potential of the technology.

### Example 8: Exemplary Phosphate-Solubilizing Bioreactor systems

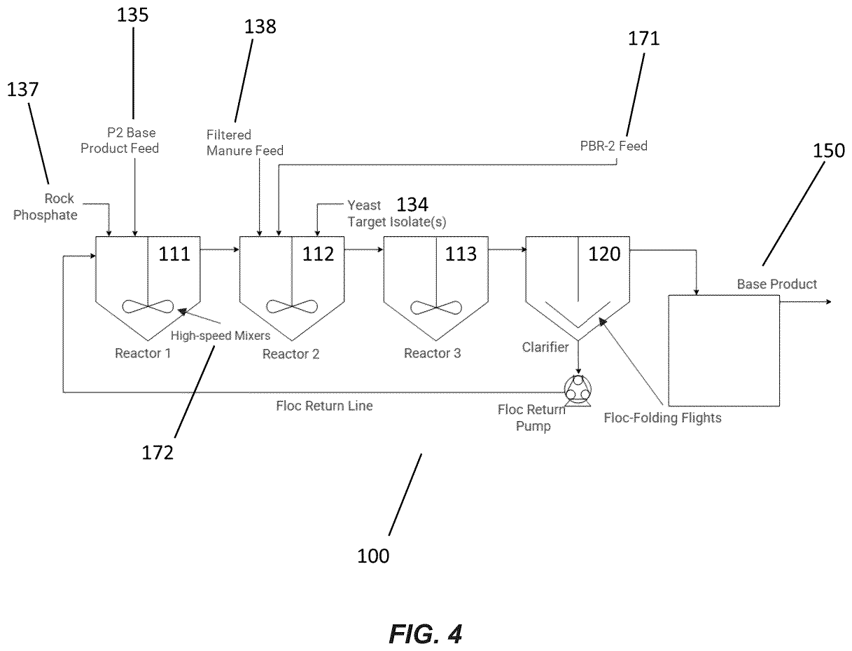

The PST-sIP serialized system was developed to improve phosphorus use efficiency (PUE) in plants. The approach was a microbial and metabolite enrichment of PUE-associated microbes and metabolites from the microbial inoculum and hydraulic source. The system used the base product of P2 technology as the hydraulic input for the line. The system utilized filtered manure as well as solution from the P2 reactor PBR-2 as microbial inoculums and a complex feed source. The system was a fluidized bed-based system that utilized high speed mixers to aerate each of the reactors. The system was composed of three high-aerated reactors followed by a clarifier where solids (floc) generated through the process were allowed to settle to the bottom of the cone-shaped contained used for other purposes such as returning to the front of the system as a microbial reinoculation or harvest as an intermediate product of the process. Addition of the target isolate MS2839 (as a sprayed dried spore powder) at different stages of the process (input into Reactor 1 or 2) was evaluated for optimal enrichment. The process utilized a floc return line that was connected at the bottom of the clarifier where floc was continuously returned to the front end of the system, Reactor 1, as a microbial reinoculation of the target isolate MS2839 and other microbes. A diagram of the PST-sIP system is shown in FIG. 4.

The PST-sIP continuous serialized system was operated with a continuous flow of input and from reactor to reactor. The continuous hydraulic source for the PST-sIP serialized systems was the base product from the P2 system (e.g., P2 base product feed). The total volume of working fluid within all of the reactors and clarifiers of the PST-sIP system was maintained at 60 gallons. The hydraulic rate of flow through the system was 12.00 mL/min to maintain a retention time of about 13 days and varied accordingly for a retention time from 7 to 21 days depending on production volume requirements with a hydraulic rate range between 7.50-22.5 mL/min. The PST-SIP system produced 2.85 to 8.56 gallons of base product flowing from the clarifiers per day. The PST-SIP system produced 19.97 to 59.91 gallons of base product flowing from the clarifiers per week.

In the PST-sIP system, rock phosphate was added into Reactor 1 at a range of 10.0-50.0 grams per day, based on the span of the hydraulic retention time of the system. The PST-sIP systems may not utilize simple sugars as carbon sources. Filtered manure (with a chemical oxygen demand of 1200-3000 mg/L) was added into Reactor 2 to maintain a concentration range of 250.0-2000 mL per day based on the span of the hydraulic retention time of the system. Solution from the packed-bed Reactor 2 (PBR-2 feed) from the P2 System for the PST-sIP system was added to Reactor 2 to maintain a concentration range of 500.0-4000.0 mL per day based on the span of the hydraulic retention time of the system. Saccharomyces cerevisiae (e.g., yeast) was added into Reactor 2 to maintain a concentration range of 0.50-5.0 grams per day based on the span of the hydraulic retention time of the system. Fluid from the top of the Reactor 1 was continuously flowed into Reactor 2 in a hydraulically balanced fashion. Fluid from the top of the Reactor 2 was continuously flowed into Reactor 3, and from Reactor 3 to the clarifier, in a hydraulically balanced fashion. In some cases, fluid from the top of a third reactor was flowed into a separation chamber (e.g., clarifier), where solids were allowed to settle. The supernatant (or base product) from the clarifier was continuously collected, and a portion of the floc at the bottom of the clarifier was returned to Reactor 1 at a rate of 500.0-4000.0 mL per day per day.

The PST-sIP process was an aerobic fermentation process. The working fluid in each reactor described herein was circulated within the reactor in such a way as to highly aerate the system using high-speed mixers. The dissolved oxygen in Reactor 1 ranged from 4.00-7.00 mg/L with an average of 8.23 mg/L, as it was a highly mixed reactor. Following the start of the fermentation in Reactor 2, the dissolved oxygen range was 4.00-7.00 mg/L with an average of 7.89 mg/L, in Reactor 3 was 4.00-7.00 mg/L with an average of 7.75 mg/L, and/or in the clarifier was 4.00-7.00 mg/L with an average of 7.93 mg/L.

The pH within each reactor of the PST-sIP system changed depending on the timing of feedstock addition and its consumption. In the PST-sIP system, the pH in Reactor 1 range was 7.00-9.00 with an average pH of 8.47. Following the start of the fermentation in Reactor 2, the pH range was 7.00-9.00 with an average pH of 8.31, and/or Reactor 3 was 7.00-9.00 with an average pH of 8.48. The clarifier has a pH range of 7.00-9.00 with an average pH of 7.78. The base product has a pH range of 6.8-9.00 with an average pH of 8.12.

### Example 9: Optimization of PST-sIP Process Design

A shake flask experiment was designed to study different variables that may be potentially incorporated into the design of a sIP system targeting the phosphate solubilization functionality. The experiment evaluated the possible enrichment of target isolates MS2839 (B. amyloliquefaciens) or MS1835 (B. licheniformis) in a PST-like environment. The experiment looked at the use of the hydraulic stock solution similar to that of a PST-system (P2 Base Product) or the use of water, to understand if either target isolate may be enriched in either environment. The study also considered the type of target isolate inoculum of either liquid spore preps, inoculated at a rate around 2.00E+08 cfu/ml in the shake flask or a spore powder preparation (sprayed dried) inoculated at the same concentration. Additionally, the experiment looked at the loading inputs (feeding) added to the shake flask to start the fermentation process. The “normal feeding” inputs normally added to the PST system (e.g., filtered-manure, PBR-2 feed, yeast, and rock phosphate) incorporated within range and the volume scale to the serialized system and corresponding retention time. For the filtered-manure and PBR-2 feed the incorporation rate was 1% v/v. The yeast was incorporated at a rate of 0.1% w/v. The rock phosphate was incorporated at a 0.1% w. v rate. The “supplemented feeding” describes additional feeding to the “normal feeding”. The supplemented feeding included an additional carbon source, glucose, at a 1.0% m/v addition, and an additional nitrogen source, ammonium sulfate, at a 0.1% w/v addition. The purpose of adding a “supplemented feeding” which included readily available carbon and nitrogen sources was to see if the target isolates may utilize the carbon and nitrogen sources better and therefore enrich in the shake flask rather than relying on the complex carbon and nitrogen sources found in the filtered manure, PBR2-feed, and yeast. The shake flask set up used 250 ml baffled shake flask to maintain high aeration. The total hydraulic volume of the shake flaks was 50 ml. The shake flask treatments were incubated at 30° C. and 200 rpm for a total of 14 days, similar to the hydraulic retention time of the PST system, with samples taken at 7 and 14 days. Below, Table 19 shows the experimental design and set up of the experiment.

Analysis of the shake flask solutions focused on heated (pasteurized) samples to determine the abundances of the target isolate spores in the solution at day 7 and at completion of the shake flask experiment, day 14. Standard dilution plating of the shake flask samples was carried out to determine bacterial spore abundance and potentially to retrieve target isolates. Briefly, samples of the shake flask collected at 7 and 14 days were pasteurized (heated to 80° C. for 15 minutes) to remove non-spore forming bacteria and plated on the same microbial nutrient media After pasteurization, the samples were passed through a series of 10-fold dilutions and 0.1 mL plated on standard microbial nutrient media. The microbial nutrient media was ¼ strength TSA (Tryptic soy agar, BD, Difco #236920). The plates were incubated at 30° C. for up to seven days and then total colony forming units (CFUs) were determined along with the target isolate counts based on bacterial morphology.

TABLE 19
Treatments and Application Rates for Long-Term Greenhouse Study

| Shake | Target | Hydraulic Stock | Inoculant | |
|---|---|---|---|---|
| Flask | Isolate | Solution | Medium | Feeding |
| A | MS2839 | P2 Base Product | Liquid | Normal Feeding |
| B | MS2839 | P2 Base Product | Liquid | Supplemented Feeding |
| C | MS2839 | P2 Base Product | Powder | Normal Feeding |
| D | MS2839 | P2 Base Product | Powder | Supplemented Feeding |
| E | MS2839 | Water | Liquid | Normal Feeding |
| F | MS2839 | Water | Liquid | Supplemented Feeding |
| G | MS2839 | Water | Powder | Normal Feeding |
| H | MS2839 | Water | Powder | Supplemented Feeding |
| I | MS1835 | P2 Base Product | Liquid | Normal Feeding |
| J | MS1835 | P2 Base Product | Liquid | Supplemented Feeding |
| K | MS1835 | P2 Base Product | Powder | Normal Feeding |
| L | MS1835 | P2 Base Product | Powder | Supplemented Feeding |
| M | MS1835 | Water | Liquid | Normal Feeding |
| N | MS1835 | Water | Liquid | Supplemented Feeding |
| O | MS1835 | Water | Powder | Normal Feeding |
| P | MS1835 | Water | Powder | Supplemented Feeding |

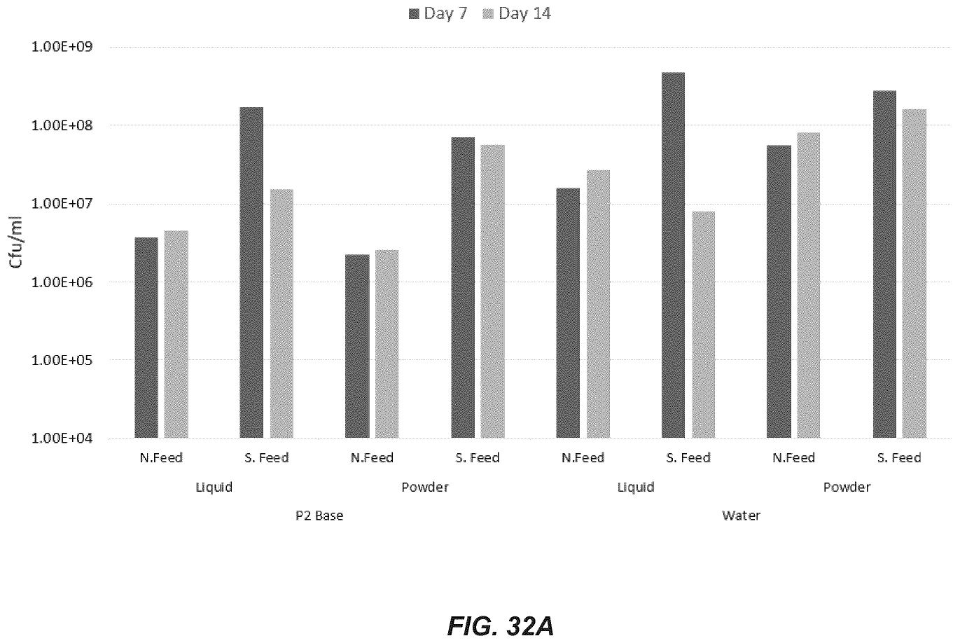

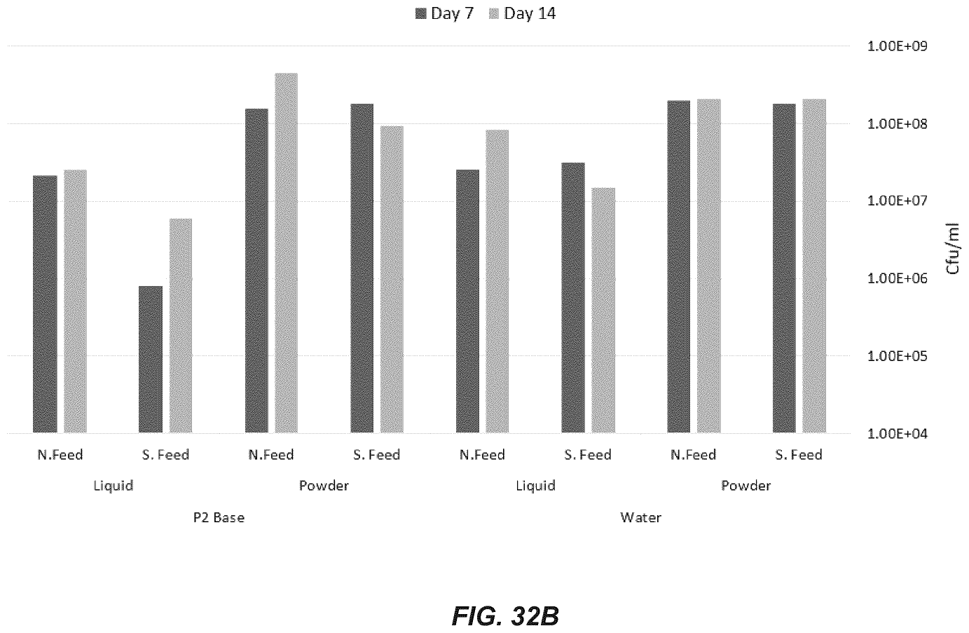

For analysis, treatments were separated based on normal feeding “N. Feed” and supplemented feeding “S. Feed”, followed by different microbial inoculation, “Liquid” or “Powder” inoculation, and separated by either use of P2 base product “P2 Base” or water as the hydraulic stock solution. FIGS. 32A-32B show the spore counts for MS2839 and MS1835. Spores were detected on ¼ strength TSA plates incubated at 30° C. for seven days for samples taken at day 7 and at completion of the experiment, day 14.

MS2839 showed the greatest spore counts all on Day 7 with the following treatment conditions: 1) supplemental feed, P2 base hydraulic stock solution, and liquid inoculation; 2) supplemental feed, water hydraulic stock solution, and liquid inoculation; and 3) supplemental feed, water hydraulic stock solution, and powder inoculation (FIG. 32A).

MS1835 showed high spore counts with multiple treatment combinations. MS1835 spore counts were the greatest at Day 14 with normal feed, P2 base hydraulic stock solution, and powder inoculation. MS1835 showed high spore counts on Days 7 and 14 with normal and supplemental feeds, water hydraulic stock solution, and powder inoculation (FIG. 32B).

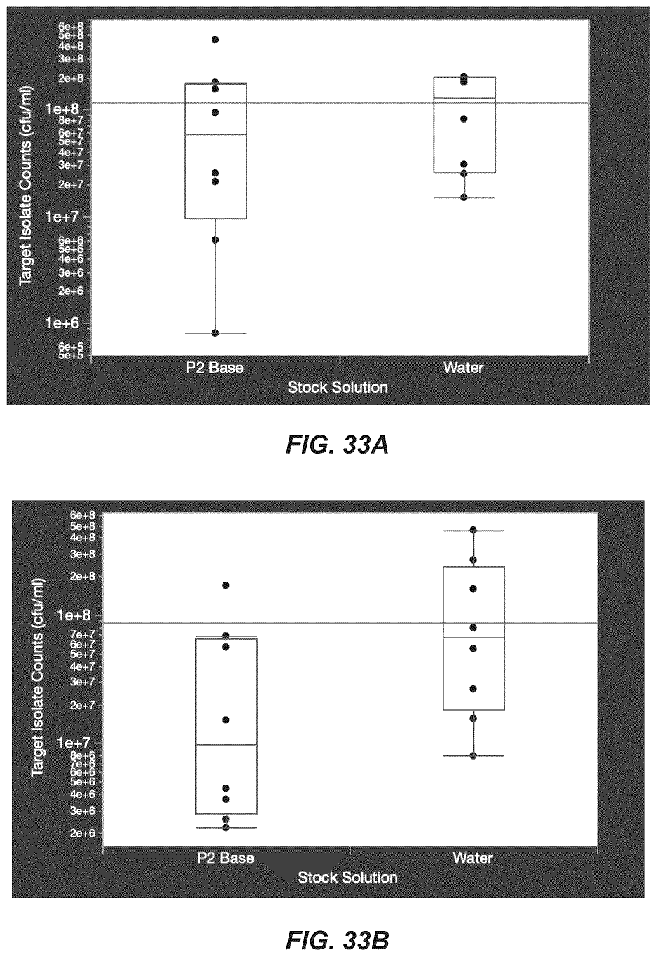

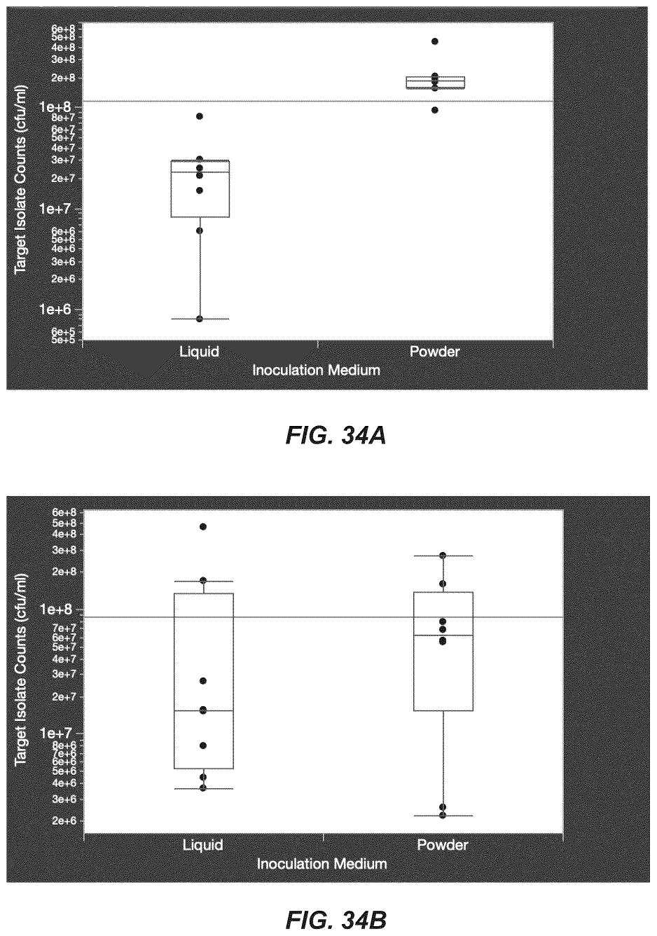

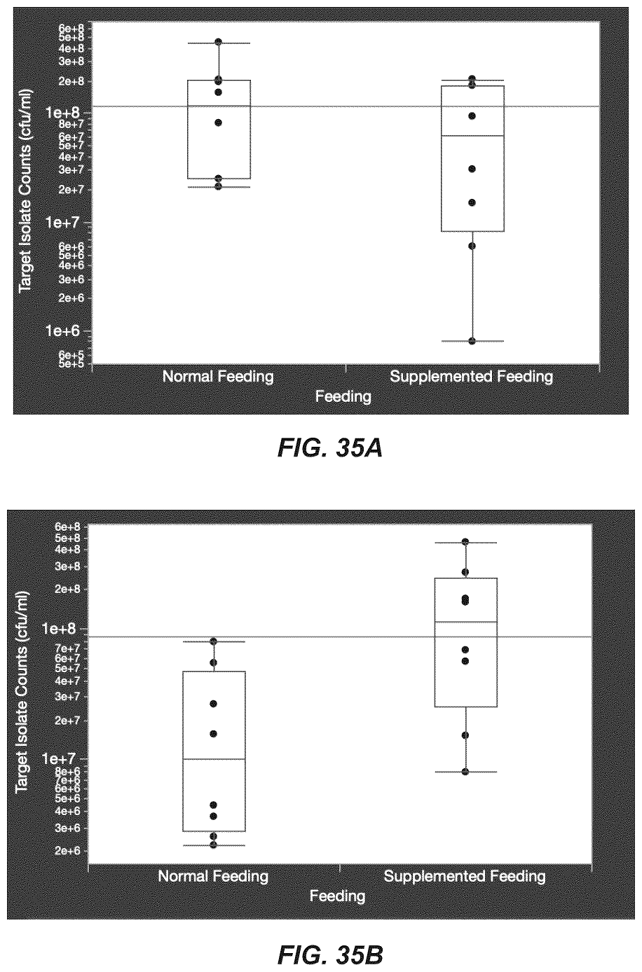

Each of the factors that were studied in this experiment were analyzed to understand what impact, if any, did it have on the target isolate and its enrichment in the shake flask. Data from both day 7 and day 14 samples were combined as well as treatments that incorporated similar factors to get a general trend. The non-parametric Wilcoxon rank test (p<0.05) was used to look at the statistical inference of each of the factors used in the experiment using the statistical analysis software, JMP. The utilization of either the P2 base product or water as the hydraulic stock solution in the shake flask did not seem to have a strong impact on the concentration for both target isolates detected in the shake flask solutions. However, there was a slight trend favoring water as the hydraulic stock solution (FIGS. 33A-33B). The utilization of using powder (spray dried) inoculum for MS1835 had a significant impact over the use of liquid medium inoculation (p<0.05) (FIG. 34A). On the other hand, there was no impact on the use of either powder or liquid medium inoculation for MS2839 (FIG. 34B). The use of supplemented feeding had no significant impact on increasing or decreasing the concentration of MS1835 (FIG. 35A). However, there was a significant increase when feeding was supplemented with the additional glucose and ammonium sulfate for MS2839 (p<0.05) (FIG. 35B).

### Example 10: Efficacy of PST-sIP Products-Plant Growth Promotion Capacity

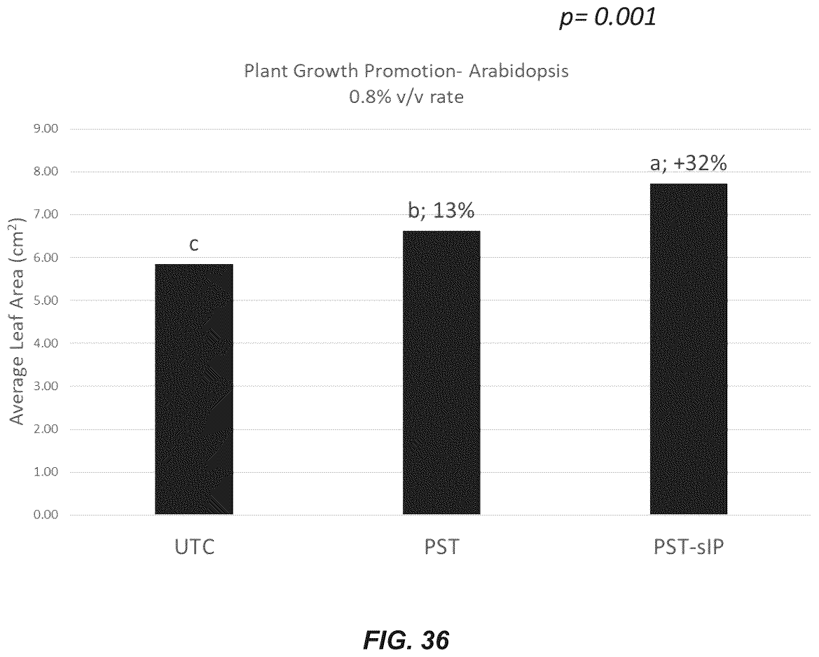

In this experiment, a continuous serialized isolate production (sIP) system was used to produce a biostimulant that promotes phosphate solubilization when applied to plants and/or soil. The sIP systems described herein may be a series of fluidized bed reactor (FBR) containers hydraulically connected with a continuous, hydraulically balanced flow from one container to the other. The FBRs in the sIP systems described in these Examples were maintained at aerobic conditions. A PST-sIP system for phosphate solubilization properties was used. The feed into the first FBR of the PST-sIP system was rock phosphate and a hydraulic source. The hydraulic source for the PST-sIP system was a feed into the first FBR of the PST system including a product of an anaerobic digestion of cow manure by a packed bed reactor system. The packed bed reactor system comprised six packed bed reactors, for a total of six PBRs maintained under anaerobic conditions with continuous, hydraulically balanced flow through each of the PBRs. The outflow from the top of the sixth PBR can be referred to herein as the “P2 base product,” which was used as the hydraulic input into the PST system. When the PST-sIP system was initially established, an inoculum of bacterial strain MS2839 was added in the first FBR. Without adding additional MS2839, an enriched population of MS2839 was maintained in the first FBR and throughout the system as the system continuously operated. Fluid from the top of the first FBR flowed into the second FBR in a hydraulically balanced fashion. Fluid from the top of the second FBR was continuously flowed into a third FBR in a hydraulically balanced fashion, and fluid form the top of the third FBR was continuously flowed into a fourth FBR in a hydraulically balanced fashion. Fluid from the top of the fourth FBR was flowed into a separation chamber, where solids were allowed to settle. The supernatant PST-sIP biostimulant product was collected, and the settled floc was returned to the first FBR. PGP of the PST-sIP supernatant was tested in Arabidopsis, at a rate of 0.8% v/v. Arabidopsis thaliana (genotype Col-0) seedlings were germinated and grown for 7 days on ½ strength MS media (Murashige & Skoog Basal Medium with Vitamins, M519) plates in the growth chamber before transplanting into rockwool cubes for plant growth assays. Rockwool was used as an inert substrate for growing Arabidopsis in a hydroponic-like system. The rockwool cubes may be fitted into a 6-well tray and each treatment consisted of three replicate well trays. Eight 1 L ½ strength MS media (Murashige & Skoog Basal Medium with Vitamins, M519) solutions were made. The test substance was then added and the pH of the solution was raised to 5.7. Forty mL of each 1 L treatment solution was dispensed to each rockwool cube. Then, each wetted cube received one Arabidopsis seedling. Arabidopsis plants grew on an LED light cart for 12 days. Leaf area was determined using the ImageJ software package. All the data collected were put through JMP for ANOVA analysis (p=0.001, a=0.05). PST-sIP supernatant showed greater PGP capacity (as average leaf area) compared to that of UTC and PST SUP (described below) (FIG. 36).

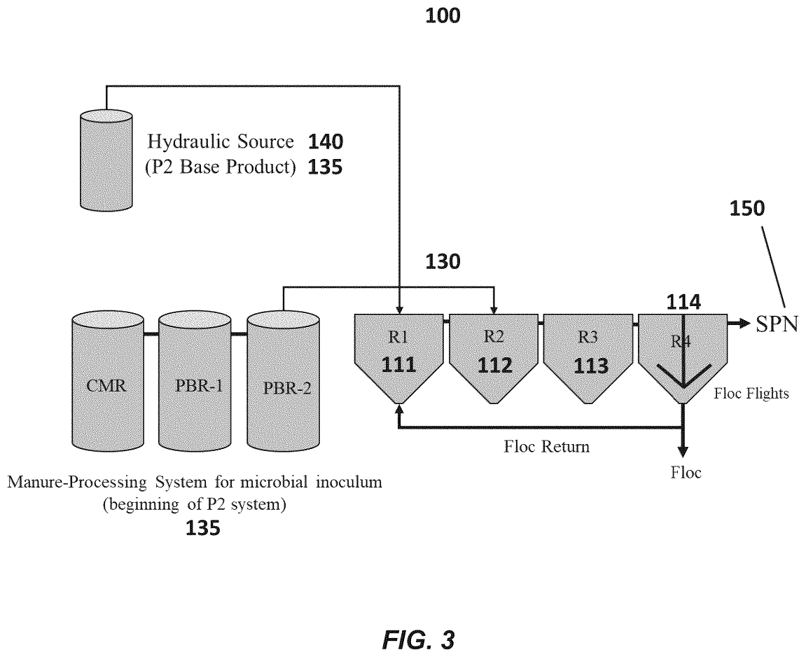

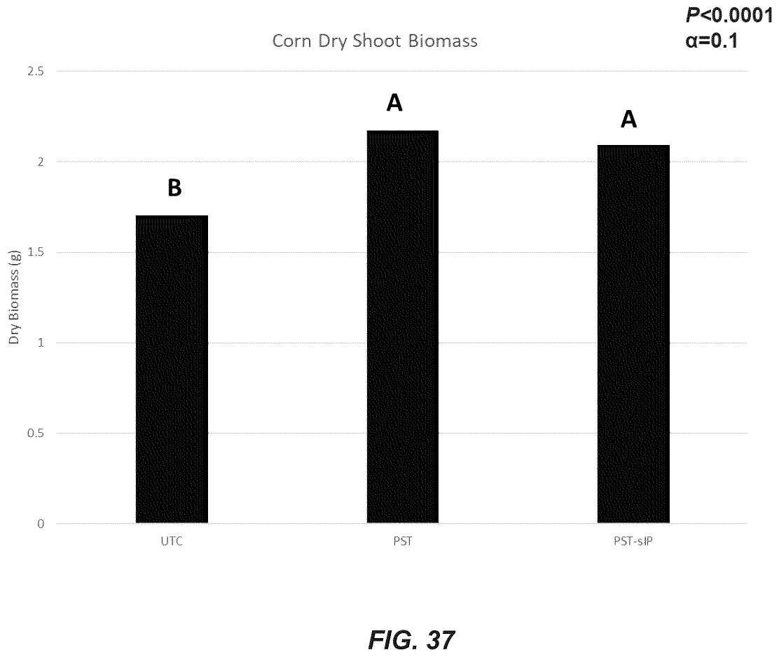

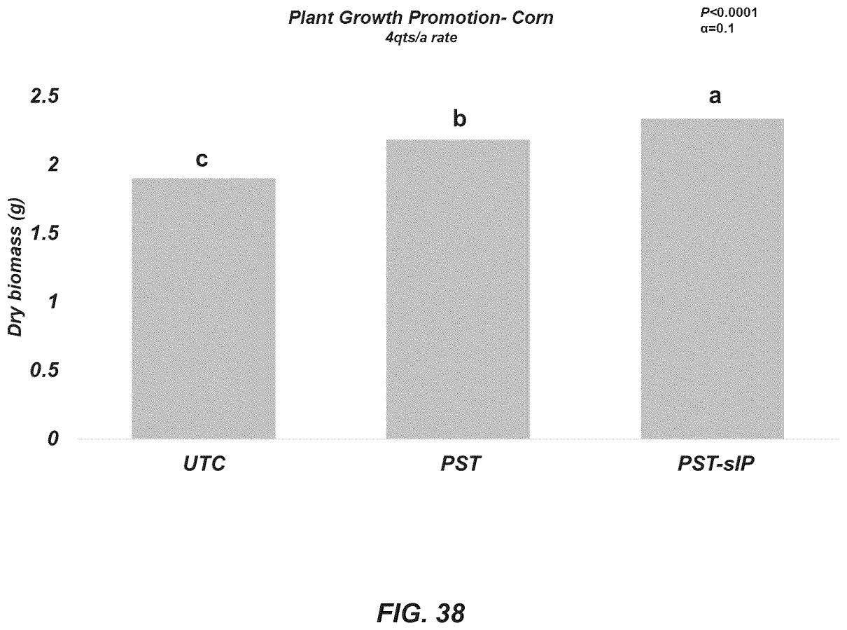

In some experiments described herein, the PST-sIP biostimulant is compared to a product of a bioreactor system referred to as a PST bioreactor system. Unlike a PST-sIP system, a PST bioreactor system is not inoculated with a population of a phosphate solubilizing microbe (e.g., MS2839). The PST bioreactor system is an aerobic fluidized bed bioreactor system as depicted in FIG. 3. Aerobic conditions are defined as DO>2 mg/L. The core of the system comprised for reactor containers (R1 to R4) arranged in series, each one flowing into the next in a hydraulically balanced fashion. The input into the first reactor (R1) was P2 base product, 200 mesh rock phosphate particles, and a return feed of floc from a later reactor in the system. This mixture was incubated in the first reactor and continuously flowed at a constant rate into the second reactor. In addition to the inflow from the first container, a fluid feed mixture containing a microbial consortium, filtered cow manure (at one liter/day), and Saccharomyces cerevisiae yeast (at 2g/day), was introduced into the second container R2. The microbial consortium was prepared by an anaerobic packed bed reactor system depicted in FIG. 5 of US2013/03244061 (a “P2 system”), with the outflow of the second packed bed reactor (PBR2) serving as the source of the microbial consortium introduced into R2 in the PST system, as depicted in FIG. 3. The feedstock for the P2 system was a mixture of filtered cow manure (with a range Chemical Oxygen Demand of 1200-3000 mg/L), Saccharomyces cerevisiae yeast, and water, mixed together in a complete mix reactor (CMR in FIG. 3) flowing into a packed bed reactor (“PBR”) container that comprised a scaffold of solid supports on which microbes can grow in biofilms. The fluid was incubated in the first packed bed reactor (PBR-1 in FIG. 3) as a continuous flow of the fluid from the top of the first PBR flowed into a second PBR in a hydraulically balanced fashion (i.e., the rate of outflow matched the rate of inflow of feedstock). The fluid from the first PBR was incubated in the second PBR (PBR-2 in FIG. 3) in the same fashion as in the first PBR, with the rate of outflow from the top of the second PBR matching the rate of inflow from the top of the first PBR. The outflow from the top of the second PBR (the “PBR2 product”) was used as the microbial inoculum provided to the second fluidized bed reactor (“FBR”) of the PST system. The FBRs of the PST system did not comprise solid scaffold supports. The fluid within the reactors was circulated at a rate that kept the rock phosphate particles suspended and that aerated the fluid by either strong continuous mixing or air sparging, thereby establishing conditions for aerobic (dissolved oxygen content of 7.47-8.17_mg/L) digestion of the digestible substrates (i.e., rock phosphate, manure, yeast, other microbes from the inoculum coming from the PBR2 system, and organic substances present in the inoculum coming from the PBR2 system). The outflow from the top of the second FBR of the PST system (R2 in FIG. 3) flowed into a third FBR (R3) in a hydraulically balanced fashion, and the outflow from the top of the third FBR flowed into a fourth FBR (R4) in a hydraulically balanced fashion. The outflow from the top of the fourth FBR is referred to herein as the PST supernatant (“SUP” or “SPN”). The outflow from the bottom of the fourth FBR (R2), referred to as floc, was flowed back to the first FBR (R1). The floc is primarily biosolids, containing high microbial content, that are generated throughout the fermentation that settle out in the clarification stage of the process. PGP capacity was also assessed by measuring corn biomass after coated-fertilizer application. Corn was grown in a Turface-Berger Peat Mix at a 1:1 ratio. There were 15 replicates per treatment in 15 cm round pots. Experimental design used a three 5*5 Latin square design. Fertilizer program used MAP application at 75 1b/A that was banded at planting. Additional applications of Jackpot Liquid Fertilizer Micronutrient Mix was applied at 0.78% were applied weekly. Coating of treatment was performed by spraying treatment on to MAP prills and letting them dry overnight. Fertilizer coated with PST-sIP supernatant produced significantly greater dry biomass than UTC treated plants (FIG. 37). In another experiment, PGP was assessed by measuring corn biomass after a greenhouse in-furrow application. Corn was grown in a Turface-Berger Peat Mix at a 1:1 ratio. There were 15 replicates per treatment in 15 cm round pots. Experimental design used a three 5*5 Latin square design. A seed was sowed in two inches below the top of the soil of the pot and treatment was applied at the top of the seed. A total delivery volume of 0.5 mLs was delivered to the seed with the treatment application incorporated at the application rate. Dry biomass was also quantified from corn which was treated in-furrow across the same systems at a rate of 4 qt/A. PST-sIP treated plants produced significantly greater dry biomass than that from UTC plants (p<0.0001, a=0.1) and PST supernatant (FIG. 38). Plant height was recorded using a PlantGazer plant phenotyping system (Confluence Research & Development, LLC). Both the PST-sIP and PST supernatants showed an increase in plant height over the untreated control

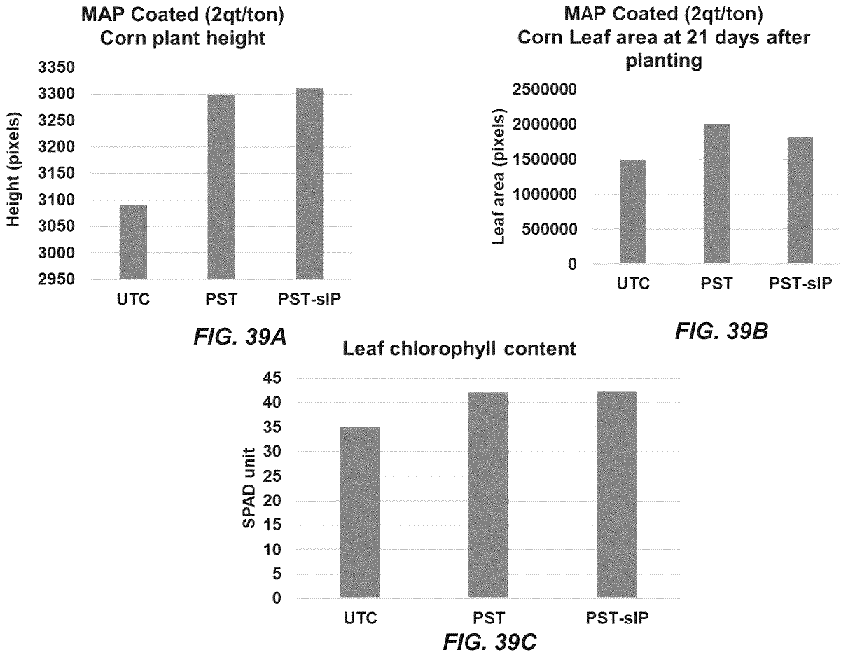

(FIG. 39A). Plant leaf area was recorded using a PlantGazer plant phenotyping system. Both the PST-sIP and PST supernatants showed a significant increase in leaf area over the untreated control (FIG. 39B). The leaf chlorophyll contents were measured from the topmost fully expanded leaf using a Chlorophyll Meter, SPAD (Soil Plant Analysis Development-502, Konica Minolta, Tokyo, Japan). The middle leaf position was selected for measuring the leaf chlorophyll contents to prevent the variation and three measurements were taken for each leaf. PST-sIP and PST supernatants showed a significant increase in chlorophyll content over the UTC (FIG. 39C). The results demonstrate a synergistic effect from providing an established population of a phosphate-solubilizing bacterial strain in the bioreactor system.

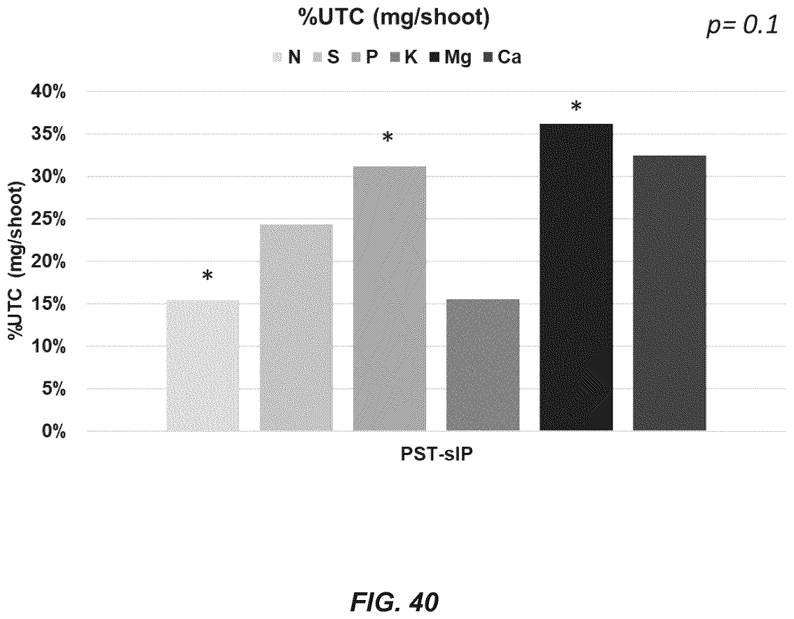

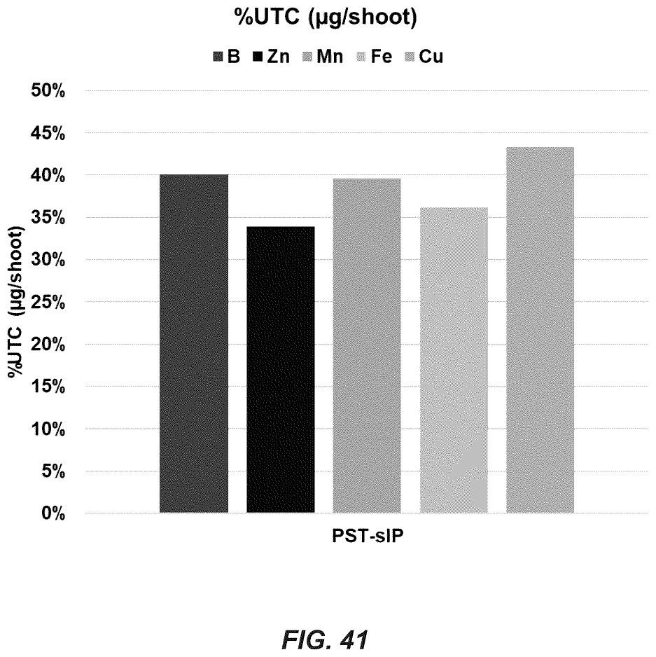

From the experimental design above, corn shoot macronutrients from UTC and PST-sIP supernatant coated-fertilizer treated plants were analyzed. Treatment was coated at 2 qt/ton to MAP and left to dry overnight. PST-sIP treated plants showed increased uptake across all six macronutrients compared to UTC. Both products showed significantly increased uptake of nitrogen (p=0.03), phosphorus (p=0.009), and magnesium (p=0.013) at 2 qt/t rate (FIG. 40). Corn shoot micronutrients were similarly tested across systems. PST-sIP supernatant showed significantly increased uptake of copper (p=0.02) at 2 qt/t rate (FIG. 41).

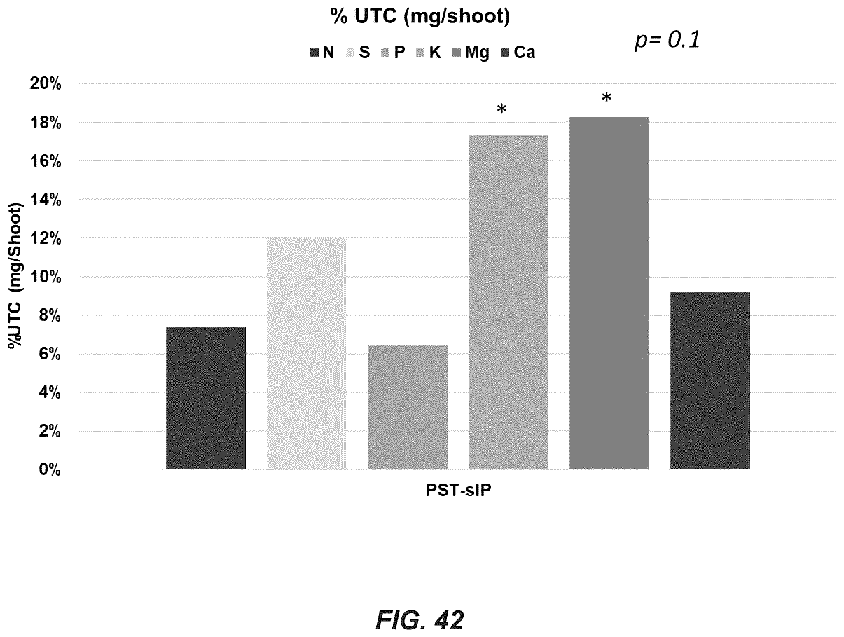

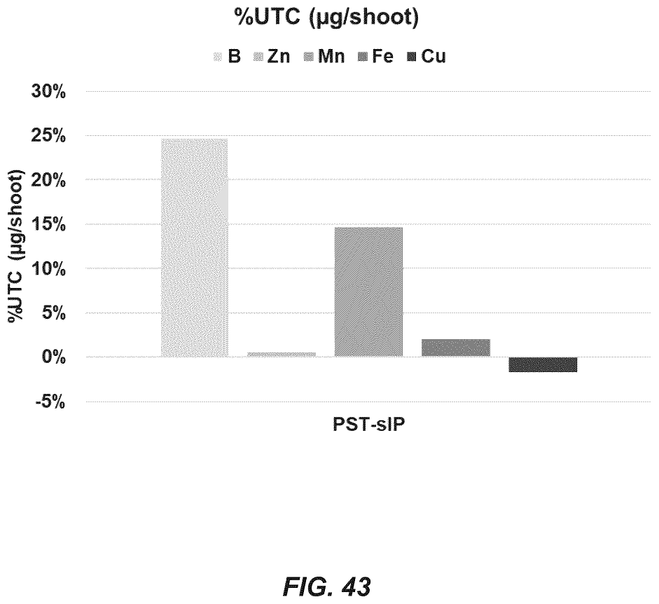

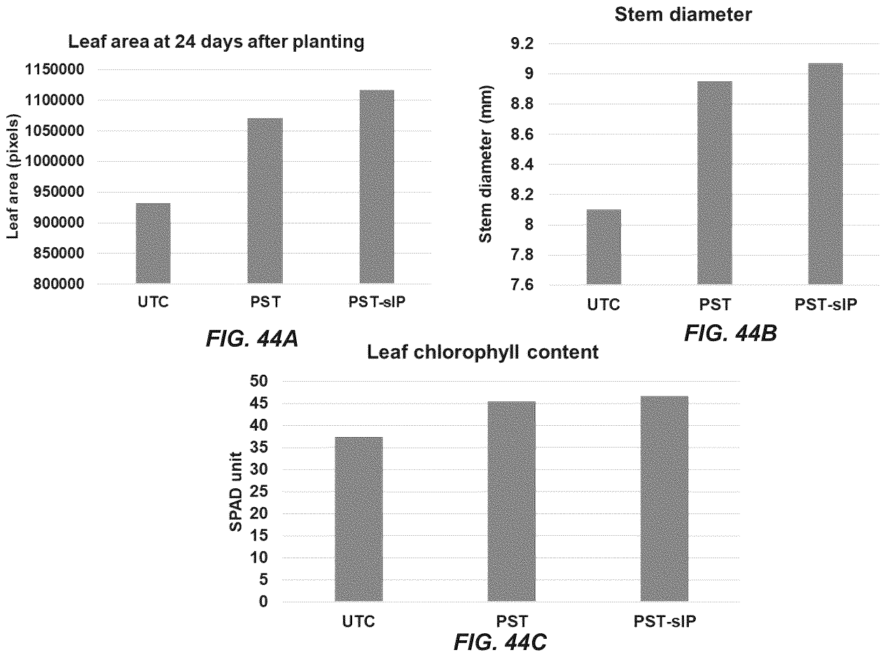

From the experimental design above, shoot macronutrients from corn treated in-furrow with PST-sIP were also analyzed. PST-sIP treated plants showed increased uptake across all six macronutrients at 4 qt/A rate (FIG. 42). In the same experiment, shoot micronutrients were similarly analyzed. PST-sIP treated plants showed increased uptake of boron and manganese (FIG. 43). The plant leaf area was recorded using a PlantGazer plant phenotyping system. The PST and PST-sIP supernatants showed a significant increase over the untreated control (FIG. 44A). The stem diameter was measured using 0-40″/1000 mm Xtra-Range Electronic Caliper. PST-sIP and PST supernatants showed a significant increase in stem diameter over the untreated control (FIG. 44B). The leaf chlorophyll contents were measured from the topmost fully expanded leaf using a Chlorophyll Meter, SPAD (Soil Plant Analysis Development-502, Konica Minolta, Tokyo, Japan). The middle leaf position was selected for measuring the leaf chlorophyll contents to prevent the variation and three measurements were taken for each leaf. PST-sIP and PST supernatants showed a significant increase in chlorophyll content over the untreated control (FIG. 44C).

Results of the experiments demonstrate a synergistic effect resulting from including an inoculum of a phosphate-solubilizing microbe in the bioreactor system.

### Example 11: Enrichment of Target Isolate in PST Serialized Isolate Production (sIP) Systems

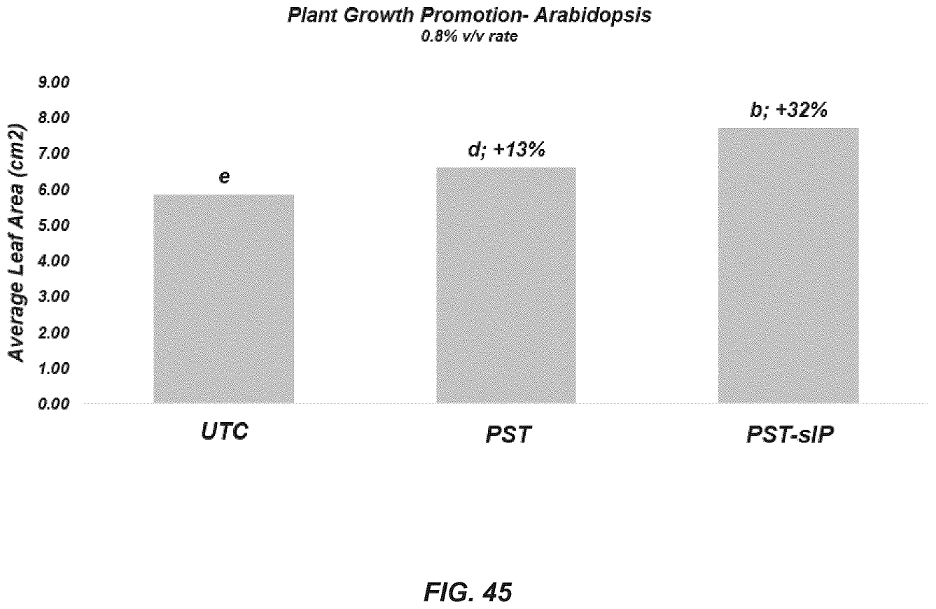

To optimize the process for a specific concentration of the target isolate in the supernatant portion of the clarifier rather than in the floc, slow rotation batch experiments with different amounts of floc were carried out. Slow mixing (50 rpm) was used not to resuspend the floc into the supernatant but rather to keep the interphase between the supernatant and floc more fluid. The concentration of MS2839 spores in the supernatant increased by over a log (FIG. 45) when slow mixing was used. Additionally, more MS2839 was released into the supernatant than other spore formers. The results show that with slow mixing, the target isolate concentration in the supernatant is able to increase over a span of 48 hours with different amounts of floc. Table 20 shows that increase of other spore-forming bacteria as well as MS2839 increasing in concentration found in the supernatant.

TABLE 20
Concentration increase from initial start of experiment of MS2839
or other spore-formers in the supernatant after 48 hours.

| % Increase | MS2839 | Other Spore Formers |
|---|---|---|
| 5% Floc | 651% | 152% |
| 25% Floc | 500% | 233% |

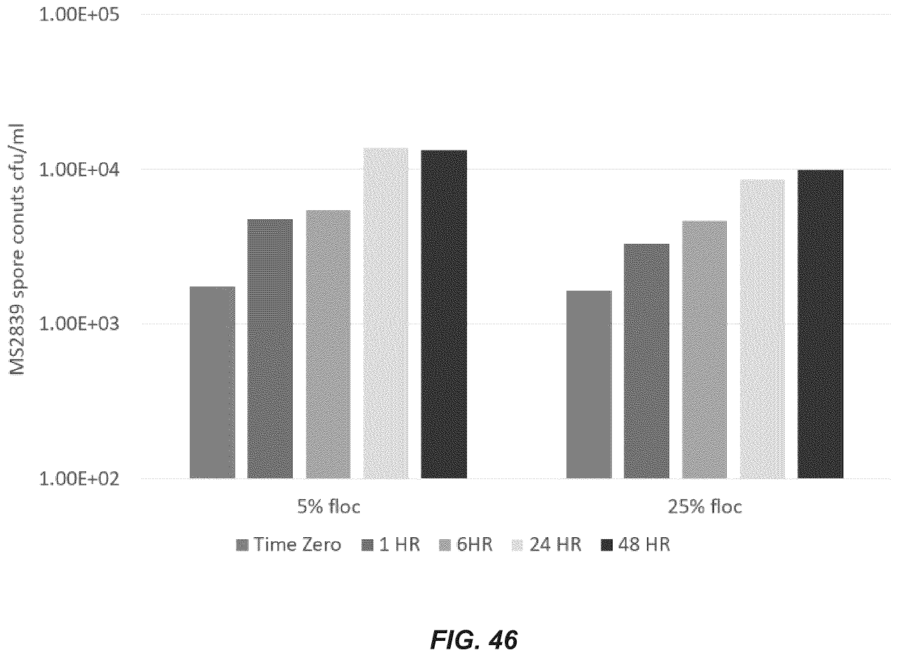

These findings were implemented using floc folding wipers in the fourth FBR of the PST-sIP system to evaluate the isolate-floc interaction and to release more target isolate, MS2839, into the supernatant (SPN) from the floc in a serialized system. Floc was continuously returned to the front end of the system to reinoculate the system with MS2839 and other microbes in the floc from the outflow of the fourth FBR. The retention time of the system was approximately 14 days. Initial inoculation of MS2839 was at a rate of 2×10⁸ cfu/ml in the second FBR of the system. Floc showed greater counts of target isolate over time. FIG. 46 shows MS2839 counts increasing over time in supernatant (SPN) after the floc folding mechanism was implemented. Thus, the floc folding mechanism can contribute to maintaining a population of target isolate (e.g., MS2893) in a bioreactor system over long periods of time without additional amounts of the target isolate being added from outside the system, and can counteract the tendency of the target isolate population to diminish over time.

A second experiment was conducted to apply the learnings from the lab-scale experiment that evaluated the release of target isolates immobilized in the floc using slow folding flights in a whole broth solution. From the previous experiment, the introduction of a folding movement increased the concentration of the target isolate MS2839 over time in the supernatant phase of the whole broth solutions while keeping the floc portion of the whole broth settled preventing resuspension. These results were then translated and applied into a clarifier operation. For this application, floc folding flights were implemented into the last stage of the continuous process in the clarifier chamber. In this stage, biosolids may settle out of the working solution gravimetrically and collect at the bottom of the cone-shaped clarifier. The aim is to release more of the target isolate that is retained in the floc portion of the clarifier back into the supernatant phase (base product) and therefore maintain a higher concentration of the target isolate in the base product for a longer period. The floc flights were installed in the clarifier, where the flights were positioned in such a way that they rotated along the walls of the cone portion of the clarifier, rotating slowly, folding the floc. This was not used to resuspend the floc into the supernatant but rather to keep the interphase between the supernatant and floc more fluid, similar to the previous experiment. To evaluate the potential application of the floc flights, the spore concentration of target isolate MS2839 was measured when the system did not have floc folding flights installed for 6-8 months and 6-8 months of data recording the concentration of the target isolate MS2839 spores in the base product when the floc folding flights were installed and operating

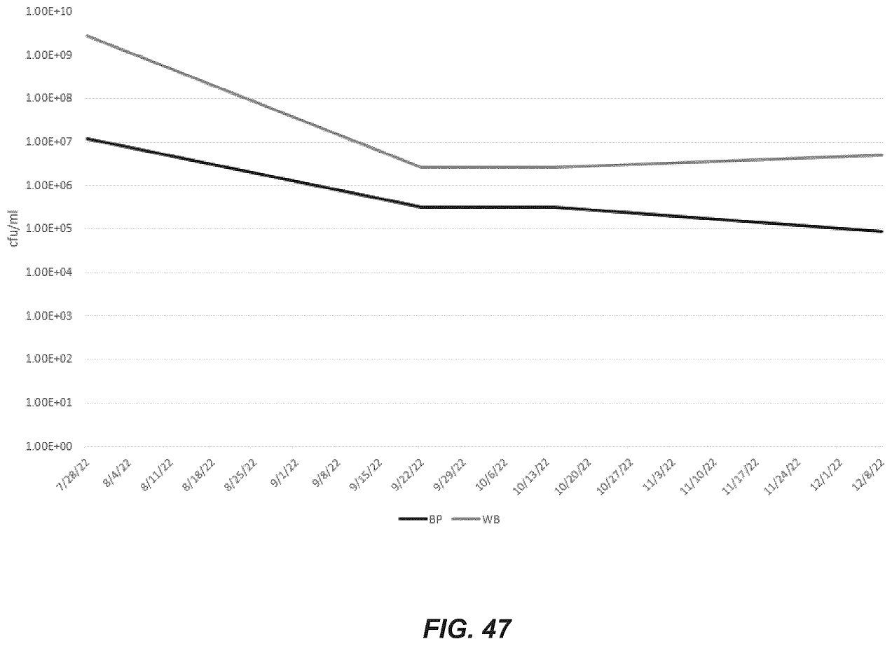

With the addition of the floc flights in the clarifier, the concentration of the target isolate MS2839 in the base product was maintained at a higher concentration for a longer period (FIG. 47). By the sixth month, the concentration MS2839 in the base product of the system that did not have floc-folding flights was recorded at 4.77E+02 cfu/ml. In comparison, by the seventh month, the concentration of MS2839 spores in the base product when the floc flights were installed was about 2.50E+04 cfu/ml. Through the use of the floc flights, the target isolate was released from the floc and into the supernatant (e.g., base product), maintaining the concentration of the target isolate in the base product at a higher concentration for longer periods of time.

### Example 12: Nitrogen Use Efficiency Bioreactor system Using Plant Growth Promoting Isolates

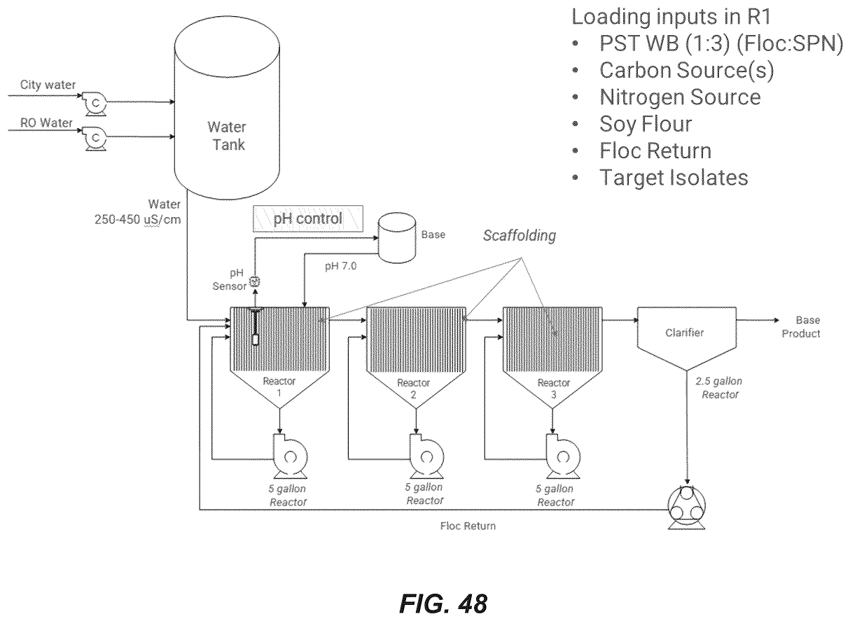

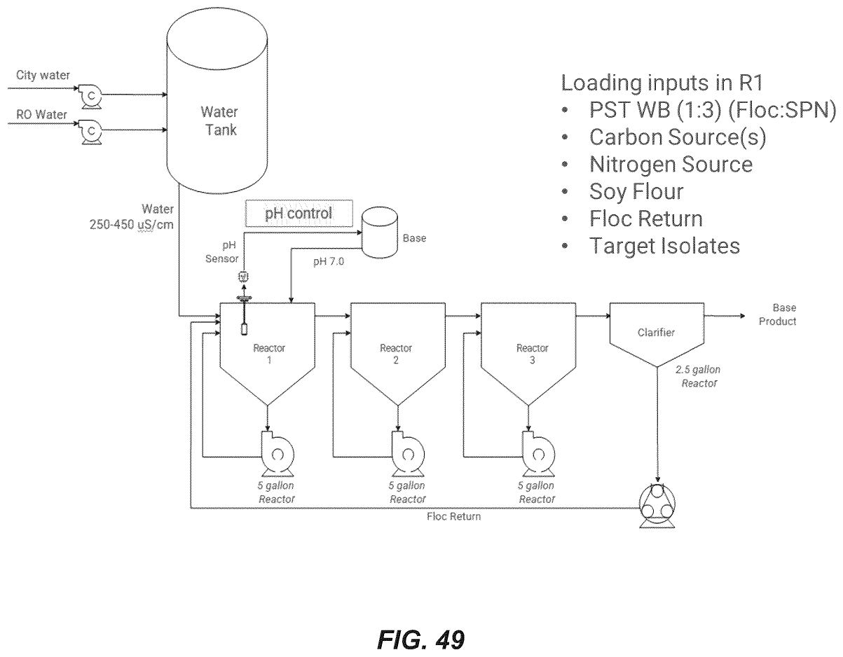

Four separate systems NTS-1.1, NTS-1.2, NTS-1.3, and/or NTS-1.4 (NTS 1.0 systems) for production of nitrogen use efficiency promoting products by digestion of organic feedstock were built and operated. These four systems were down-flow systems. Each of the systems comprised a series of three 5-gallon reactor chambers (Reactor 1, Reactor 2, and/or Reactor 3) with continuous flow of working fluid from one reactor to the next and ultimately to the clarifier container, which is a 2.5 gallon reactor. Initially, a water tank is connected to Reactor 1 and provides continuous flow of water to Reactor 1 at an electrical conductivity ranging from 250-450 microsiemens/centimeter (μS/cm). The water tank comprised water from a city water source and a reverse osmosis (RO) water source. Reactor 1 is also coupled to a pH sensor, pH controller, and/or automatic buffer addition system to maintain the pH at 7.0±0.1. When the pH of the working solution in Reactor 1 gets below the set threshold, there is an automatic base (3M NaOH) addition until a pH of 7.0 or higher is reached. In some cases, the working fluid in each reactor is recirculated from the bottom of the reactor and pumped back to just below the surface of the same reactor to maintain homogeneous conditions within the working solutions. Biosolids (floc) is generated through the process. The solids are collected in the clarifier container where a portion is recycled back upstream of the process. The three reactors of the NTS-1.1 and/or NTS-1.3 systems were packed bed reactors with scaffolding within the reactors. The three reactors of the NTS-1.2 and/or NTS-1.4 systems were fluidized bed reactors, which lacked the scaffolding. In some cases, the reactors comprised a distribution ring that is subsurface of the discharge volume, reduced the amount of surface disruption, and kept the environment in the reactor anaerobic. A diagram of the NTS-1.1 and NTS-1.3 systems is shown in FIG. 48. A diagram of the NTS-1.2 and NTS-1.4 systems is shown in FIG. 49.

As shown in FIG. 48 and FIG. 49, fluid from the top of Reactor 1 continuously flowed into Reactor 2 in a hydraulically balanced fashion. In each of NTS-1.1, NTS-1.2, NTS-1.3, and NTS-1.4, glucose and ammonium sulfate were added to Reactor 1 to maintain a concentration range of 0.2-2.0% w/v and 0.02-0.2% w/v, respectively, based on the span of the hydraulic retention time of the system. Malate was also added to Reactor 1 of the NTS-1.3, and/or NTS-1.4 systems at a concentration range of 0.2-2.0% w/v based on the span of the hydraulic retention time of the system. A concentration range of 0.5-4.5% v/v of Whole Broth of a Phosphate Solubilization Technology, PST WB (in a ratio of 1:3 floc: supernatant (SPN)) or the Whole Broth of a Water Based Phosphate Solubilization Technology, PwST WB (in a ratio of 1:3 floc: SPN) was also added to Reactor 1 on the span of the hydraulic retention time of the system. Soy flour, calcium carbonate were added to Reactor 1 at a concentration range of 0.2-3.0% w/v and, respectively, based on the span of the hydraulic retention time of the system. Fluid from the top of the Reactor 2 was continuously flowed into Reactor 3 in a hydraulically balanced fashion. In some cases, fluid from the top of a third reactor was flowed into a separation chamber (clarifier), where solids were allowed to settle. The hydraulic rate was 4.6 mL/min. The supernatant (or base product) from the clarifier was continuously collected, and a portion of the floc at the bottom of the clarifier was returned to Reactor 1 at a rate of 1.32 L/day.

Two additional scaled up separate systems NTS-2.2 and/or NTS-2.3 (NTS 2.0 systems) were built based on the NTS-1.4 design and process for optimization at a larger scale. These two systems were down-flow systems. The two systems also each ran cooler than the NTS-1.1, NTS-1.2, NTS-1.3, and/or NTS-1.4 systems due to scale. For example, the NTS-2.2 and NTS-2.3 system temperature for the first reactor was around 20% lower than the temperatures from the NTS-1.1, NTS-1.2, NTS-1.3, and/or NTS-1.4 systems.

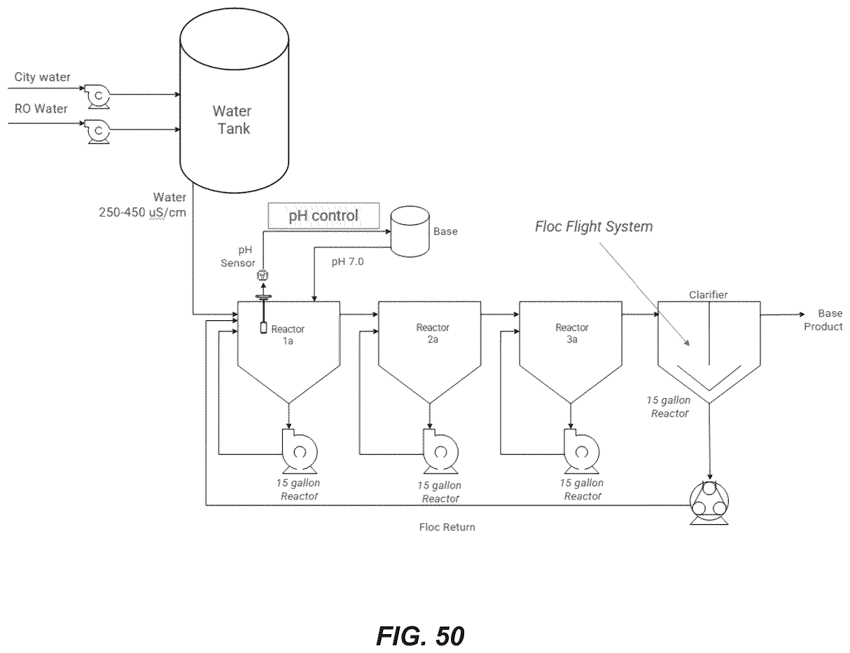

NTS-2.2 is inoculated with isolates MS3900 and/or MS3907; and NTS-2.3 is inoculated with isolates MS3900, MS3907, and/or MS4921. Each of the scaled up systems comprised a series of three 15 gallon reactor chambers (Reactor 1a, Reactor 2a, and/or Reactor 3a) with continuous flow of working fluid from one reactor to the next and ultimately to the clarifier container, which is a 15 gallon reactor. Initially, a water tank is connected to Reactor 1a and provides continuous flow of water to Reactor 1a at an electrical conductivity ranging from 250- 450 microsiemens/centimeter (μS/cm). The water tank comprised water from a city water source and a reverse osmosis (RO) water source. Reactor 1a is also coupled to a pH sensor connected to a pH controller to provide an automatic buffer addition to maintain the pH at 7.0±0.1. When the pH drops below the set point, the pump connected to the controller will add base (3N NaOH) until the pH is again within the desired threshold. In some cases, the working fluid from each reactor is recirculated within each reactor from the bottom of the reactor back to just below the surface of the working solution to maintain a homogeneous environment for the fermentation. For example, a portion of the working fluid from reactor 1a is recycled back into Reactor 1a. The three reactors of the NTS-2.2 and/or NTS-2.3 systems were fluidized bed reactors, which lacked scaffolding. In some cases, the reactors comprised a distribution ring that is subsurface of the discharge volume, which reduced the amount of surface disruption, and kept the environment in the reactor anaerobic. A diagram of the NTS-2.2 and/or NTS-2.3 systems is shown in FIG. 50. As shown in FIG. 50, fluid from the top of Reactor 1a continuously flowed into Reactor 2a in a hydraulically balanced fashion. In each of NTS-2.2 and NTS-2.3, glucose and ammonium sulfate were added to Reactor 1a to maintain a concentration range of 0.2-2.0% w/v and 0.02-0.2% w/v, respectively, based on the span of the hydraulic retention time of the system. Malate was also added to Reactor 1a of both systems at a concentration range of 0.2-2.0% w/v based on the span of the hydraulic retention time of the system. A concentration range of 0.5-4.5% v/v of Whole Broth of a Phosphate Solubilization Technology, PST WB (in a ratio of 1:3 floc: supernatant (SPN)) or the Whole Broth of a Water Based Phosphate Solubilization Technology, PwST WB (in a ratio of 1:3 floc: SPN) was also added to Reactor 1a based on the span of the hydraulic retention time of the system. Soy flour and calcium carbonate were added to Reactor 1a at a concentration range of 0.2-3.0% w/v and 0.02-0.2% w/v, respectively, based on the span of the hydraulic retention time of the system. Fluid from the top of the Reactor 2a was continuously flowed into Reactor 3a in a hydraulically balanced fashion. In some cases, fluid from the top of a third reactor was flowed into a separation chamber (clarifier), where solids were allowed to settle. The hydraulic rate for NTS-2.2 and/or NTS-2.3 were 15.77 mL/min and 11.27 mL/min respectively. The supernatant (or base product) from the clarifier was continuously collected, and a portion of the floc at the bottom of the clarifier was returned to Reactor 1a at a concentration of 4.54 L/day. The clarifier comprised a floc flight system (e.g., floc-folding flights) as shown in FIG. 2 that can improve the concentration of the added specific isolate(s) in the base product. The floc returned back to the system as a 2% v/v rate per day. Additionally, solids management was maintained at 20-25% range in the system. For example, as solids accumulate over time in the clarifier container, a range of 20-25% solids v/v is maintained. Additional floc is harvested from the system and removed.

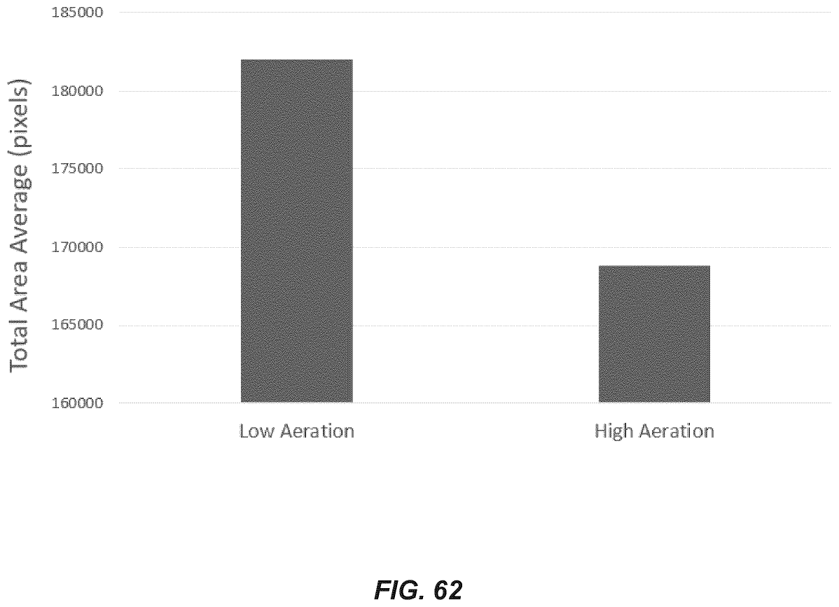

The PST WB indicated as an input into R1 (in FIGS. 6 and 7) or R1a (FIG. 62) refer to a product of a separate serialized bioreactor system (the PwST or PST system), which has as an input an aqueous feed comprising (1) a microbial consortium derived from digestion of cow manure by microbes endogenous to the manure and (2) rock phosphate. The whole broth (WB) product of the PwST system comprises supernatant and floc solids with their associated microbial consortia. The PwST WB input into the NTS systems contains floc and supernatant (SPN) in a ratio of floc to supernatant of 1:3. The carbon source input into the NTS systems comprises either glucose (NTS-1.1 & NTS-1.2) or glucose and malate (NTS-1.3,NTS-1.4, NTS-2.2, and/or NTS-2.3). Aside from the carbon source input, inputs also came from a nitrogen source and isolates. In some cases, the quantities added of the carbon and nitrogen sources to the systems are relative to the retention time of the systems and can be adjusted accordingly if the flow rate changes, since the NTS systems are continuous. The specific isolates (or targeted isolates) input into the NTS-1.1, NTS-1.2, NTS-1.3, NTS-1.4, and/or NTS-2.2 systems were MS3907 & MS3900 spores. The “target isolates” input into the NTS-2.3 system was MS3907, MS3900, and/or MS4921 spores. The “floc return” input comes from an outlet at the bottom of the clarifier (CLF) chamber.

The NTS systems were operated with a continuous flow of input and from reactor to reactor. The total volume of working fluid within all of the reactors and clarifiers of NTS-1.1, NTS-1.2, NTS-1.3, and/or NTS-1.4 systems was maintained at 17.5 gallons. The hydraulic rate of flow through these four systems was 4.6 ml/min to maintain a retention time of 10 days and can be varied accordingly for a retention time from 7 to 21 days depending on production volume requirements. The total volume of working fluid within all of the reactors and clarifiers was of NTS-2.2 and/or NTS-2.3 systems was maintained at 60 gallons. The hydraulic rate of flow through these two systems was 15.77 ml/min and 11.27 ml/min for NTS-2.2 and NTS-2.3, respectively. The retention time of the NTS-1.1, NTS-1.2, NTS-1.3, NTS-1.4, and/or NTS-2.2 systems was 10 days. The retention time of the NTS-2.3 system was 14 days. The NTS-1.1, NTS-1.2, NTS-1.3, and/or NTS-1.4 systems produced 1.75 gallons of base product flowing from the clarifiers per day. The NTS-2.2 and/or NTS-2.3 systems produced 6 gallons of base product flowing from the clarifiers per day and 4.29 gallons of base product flowing from the clarifiers per day respectively. The NTS-1.1, NTS-1.2, NTS-1.3, and/or NTS-1.4 systems produced 12.25 gallons of base product flowing from the clarifiers per week. The NTS-2.2 and/or NTS-2.3 systems produced 42 gallons of base product flowing from the clarifiers per week and 30 gallons of base product flowing from the clarifiers per week respectively. The NTS-2.2 and/or NTS-2.3 systems resulted in a 3.43 total system volume scale up. The scale up of NTS-1.4 system to NTS-2.0 systems used retention time as the constant variable to determine scale factors.

The produced NTS-1.0 base products had an average pH of 7.9-8.13, an average COD of 180-200 ppm, a total nitrogen (N) content of 0.02%, a total phosphorous (P) content of 0.00001%, and a total potassium (K) content of 0.094%. The NTS products comprised MS3907 and/or MS3900. MS3907 and/or MS3900 can appear in the biofilm within the reactors. In some cases, the NTS product may appear in other areas of the system aside from the biofilm. In some cases, isolates can contribute their metabolites to the system whether or not they are in the base product. Analysis of the bacterial content of the NTS products showed the presence of a microbial consortium enriched for nitrogen fixing bacteria. In some cases, MS4921 is expected to be present in the reactors, base product, final product, floc, and/or biofilm. The produced NTS-1.1 base products has a pH range of 7.39-8.71, and electrical conductivity (Cond) range of 0.55-2.06 mS/cm, a chemical oxygen demand (COD) range of 22-240 mg/L. The produced NTS-1.2 base products has a pH range of 7.43-8.36, and electrical conductivity (Cond) range of 0.55-1.72 mS/cm, a chemical oxygen demand (COD) range of 81.95-670 mg/L. The produced NTS-1.3 base products has a pH range of 8.05-8.66, and electrical conductivity (Cond) range of 1.63-2.64 mS/cm, a chemical oxygen demand (COD) range of 15-176 mg/L. The produced NTS-1.4 base products has a pH range of 8.11-8.88, and electrical conductivity (Cond) range of 1.63-2.64 mS/cm, a chemical oxygen demand (COD) range of 89.65-815 mg/L. The produced NTS-2.2 base products has a pH range of 7.62-8.65, and electrical conductivity (Cond) range of 2.6-4.37 mS/cm, a chemical oxygen demand (COD) range of 190-400 mg/L. The produced NTS-2.2 base products has a pH range of 7.74-8.65, and electrical conductivity (Cond) range of 2.59-4.37 mS/cm, a chemical oxygen demand (COD) range of 230-400 mg/L.

The working fluid in each reactor described herein is circulated within the reactor in such a way as to minimize surface disruption, which maintains the dissolved oxygen within the reactors between 0.14 and 0.45 mg/L. The NTS-1.1, NTS-1.2, NTS-1.3, NTS-1.4, NTS-2.2 and/or NTS-2.3 systems encompasses a microaerobic process. In the NTS-1.1 system, the dissolved oxygen in Reactor 1 was 0.22 mg/L, in Reactor 2 was 0.18 mg/L, in Reactor 3 was 0.2 mg/L, and/or in the Clarifier was 0.15 mg/L. In the NTS-1.2 system, the dissolved oxygen in Reactor 1 was 0.17 mg/L, in Reactor 2 was 0.15 mg/L, in Reactor 3 was 0.15 mg/L, and/or in the Clarifier was 0.13 mg/L. In the NTS-1.3 system, the dissolved oxygen in Reactor 1 was 0.16 mg/L, in Reactor 2 was 0.14 mg/L, in Reactor 3 was 0.45 mg/L, and/or in the Clarifier was 0.13 mg/L. In the NTS-1.4 system, the dissolved oxygen in Reactor 1 was 0.15 mg/L, in Reactor 2 was 0.13 mg/L, in Reactor 3 was 0.14 mg/L, and/or in the Clarifier was 0.14 mg/L. In the NTS-2.2 system, the dissolved oxygen in Reactor 1a was 0.21 mg/L, in Reactor 2a was 0.21 mg/L, and/or in Reactor 3a was 0.2 mg/L. In the NTS-2.3 system, the dissolved oxygen in Reactor 1a was 0.2 mg/L, in Reactor 2a was 0.22 mg/L, and/or in Reactor 3a was 0.19 mg/L.

### A. NTS-1.5 System

In an example, NTS-1.5 is a continuous serialized system that was built to look at the base technology without the inoculation of the target isolates. The system was designed to compare the base technology performance, efficacy, and chemical profile, when no target isolates were added to the process. The process most closely resembled the NTS-1.4, in design, process ranges, and all inputs (except the target isolates) added at similar concentration ranges, based on the hydraulic retention time of the system. The system was also a fluidized bed-based system that utilized PwST WB at a 1:3 floc: supernatant ratio as the microbial inoculum. This specific system comprised three reactors that used a down-flow orientation of the hydraulic re-circulation within each reactor and that were followed by a clarifier container from which the byproducts were recovered, (1) the floc that was separated by gravity and (2) the supernatant that was collected as the main product output of the system. Floc that was recovered from the clarifier was returned upstream of the system to Reactor 1. Reactor 1, Reactor 2, and Reactor 3 each had a working volume of 5 gallons. The clarifier container of NTS-1.5 system had a working volume of 2.5 gallons. The total working volume for the NTS-1.5 system was 17.5 gallons.

In NTS-1.5, glucose and ammonium sulfate were added to Reactor 1 to maintain a concentration range of 0.2-2.0% w/v and 0.02-0.2% w/v, respectively, based on the span of the hydraulic retention time of the system. Malate was also added to Reactor 1 at a concentration range of 0.2-2.0% w/v based on the span of the hydraulic retention time of the system. A concentration range of 0.5-4.5% v/v of Whole Broth of a Phosphate Solubilization Technology, PST WB (in a ratio of 1:3 floc: supernatant (SPN)) was also added to Reactor 1 on the span of the hydraulic retention time of the system. Soy flour and calcium carbonate were added to Reactor 1 at a concentration range of 0.2-3.0% w/v and 0.02-0.2%, respectively, based on the span of the hydraulic retention time of the system. The hydraulic rate of flow through the NTS-1.5 line was about 4.6 ml/min to maintain a retention time of 10 days and could be varied accordingly for a retention time from 7 to 21 days depending on production volume requirements with a hydraulic rate range between 2.19-6.57 mL/min. The total volume of working fluid within all the reactors and clarifier of the NTS-1.5 line was maintained at 17.5 gallons.

The NTS-1.5 systems produced 12.25 gallons of base product (BP) flowing from the clarifiers per week. The produced NTS-1.5 base product had a pH range of 7.30-8.40, electrical conductivity (Cond) range of 0.50-2.46 mS/cm, and a chemical oxygen demand (COD) range of 22-300 mg/L. The working fluid in each reactor R1-R3 was circulated within the reactor in such a way as to minimize surface disruption, which maintained the dissolved oxygen within the reactors between 0.14 and 0.22 mg/L. Similar to the NTS-1.1, NTS-1.2, NTS-1.3, NTS-1.4 systems, the NTS-1.5 system encompassed a microaerobic process. In the NTS-1.5 system, the dissolved oxygen in Reactor 1 was 0.19 mg/L, in Reactor 2 was 0.18 mg/L, in Reactor 3 was 0.22 mg/L, and/or in the Clarifier was 0.14 mg/L.

The hydraulic rate of flow through the bioreactor system may be 4.60 mL/min to maintain a retention time of 10 days and can be varied accordingly for a retention time from 7 to 21 days depending on production volume requirements. Because of this, the amount of product produced per day ranged from 0.83 to 2.50 gallons. The amount of product produced per week ranged from 8.75 to 17.50 gallons.

### B. NTS-2.2 and NTS-2.3 Systems

In an example, NTS-2.2 and/or NTS-2.3 were built based on the NTS-1.4 design and process for optimization at a larger scale. Similarly as described in the above section, each of the scaled up systems comprised a series of three 15 gallon reactor chambers (Reactor 1a, Reactor 2a, and/or Reactor 3a) with continuous flow of working fluid from one reactor to the next and ultimately to the clarifier container, which is a 15 gallon reactor. Initially, a water tank is connected to Reactor 1a and provides continuous flow of water to Reactor 1a at an electrical conductivity ranging from 250-450 microsiemens/centimeter (μS/cm). The water tank comprised water from a city water source and a reverse osmosis (RO) water source. Reactor 1a is also coupled to a pH sensor connected to a pH controller to provide an automatic buffer addition to maintain the pH at 7.0±0.1. When the pH drops below the set point, the pump connected to the controller will add base (3M NaOH) until the pH is again within the desired threshold. In some cases, the working fluid from each reactor is recirculated within each reactor from the bottom of the reactor back to just below the surface of the working solution to maintain an homogeneous environment for the fermentation. For example, a portion of the working fluid from reactor 1a is recycled back into Reactor 1a. The three reactors of the NTS-2.2 and/or NTS-2.3 systems were fluidized bed reactors, which lacked the scaffolding. In some cases, the reactors comprised a distribution ring that is subsurface of the discharge volume, reduced the amount of surface disruption, and kept the environment in the reactor anaerobic. A diagram of the NTS-2.2 and/or NTS-2.3 systems is shown in FIG. 50.

The retention time of NTS 2.2 and NTS 2.3 can be set at 10 days and the hydraulic rate can be varied accordingly for a retention time from 7 to 21 days depending on production volume requirements. As shown in FIG. 50, fluid from the top of Reactor 1a continuously flowed into Reactor 2a in a hydraulically balanced fashion. The “target isolates” input into the NTS-2.2 system were MS3907 & MS3900; and the “target isolates” input into the NTS-2.3 system was MS3907, MS3900, and/or MS4921. These target isolates were inoculated into Reactor 1a. In each of NTS-2.2 and NTS-2.3, glucose and ammonium sulfate were added to Reactor 1a to maintain a concentration range of 0.2-2.0% w/v and 0.02-0.2% w/v, respectively, based on the span of the hydraulic retention time of the system. Malate was also added to Reactor 1a of both systems at a concentration range of 0.2-2.0% w/v based on the span of the hydraulic retention time of the system. A concentration range of 0.5-4.5% v/v of Whole Broth of a Phosphate Solubilization Technology, PST WB (in a ratio of 1:3 floc: supernatant (SPN)) or Whole Broth of a Water Based Phosphate Solubilization Technology, PwST WB (in a ratio of 1:3 floc: SPN) was also added to Reactor 1a based on the span of the hydraulic retention time of the system. Soy flour and calcium carbonate were added to Reactor 1a at a concentration range of 0.2-3.0% w/v and 0.02-0.2% w/v, respectively, based on the span of the hydraulic retention time of the system. Fluid from the top of the Reactor 2a was continuously flowed into Reactor 3a in a hydraulically balanced fashion. In some cases, fluid from the top of a third reactor was flowed into a separation chamber (clarifier), where solids were allowed to settle. The hydraulic rate for NTS-2.2 and/or NTS-2.3 were 15.77 mL/min and 11.27 mL/min respectively. The supernatant (or base product) from the clarifier was continuously collected, and a portion of the floc at the bottom of the clarifier was returned to Reactor 1a at a concentration of 2% v/v per day. The clarifier comprised a floc flight system (e.g., floc-folding flights) as shown in FIG. 2 that can improve the concentration of target isolated in the base product. The floc returned back to the system as a 2% v/v rate per day. Additionally, solids management was maintained at 20-25% range in the system.

In an example, as scaled-up versions of NTS 1.4, NTS 2.2 and NTS 2.3 provide conditions for enrichment of a nitrogen-fixing microbial community (Table 21) and their Base Product solutions are expected to have nitrogen fixation capacity as measured with the ARA assay. Similarly to the NTS 1.0 systems, NTS 2.2 and NTS 2.3 output solutions either intact or filter sterilized (e.g., metabolites only) are expected to provide plant growth promotion in high throughput assays with Arabidopsis as well as in greenhouse experiments in multiple crops such as corn, sorghum, fescue, wheat, canola, soybean, tomato. As a result, it is expected that there may be a positive effect on growth, yield and N content in crops grown in outdoor pot studies and field trials with crops like corn, sorghum, wheat and soybean. The produced base products of the NTS 2.2 and NTS 2.3 lines are expected to have a pH range of 7.8-8.88, electrical conductivity (Cond) range of 1.50-2.8 mS/cm, and a chemical oxygen demand (COD) range of 80-500 mg/L.

TABLE 21
Nitrogen-fixing Microbial Communities from NTS-2.2
and NTS-2.3 Solutions. Reactor 1a input inoculum refers to
the expected output of PwST WB (1:3 floc:SPN) inoculum at 1% into
Reactor 1a without any other feed components added for enrichment.

| | Reactor 1a Input | Base Product |
|---|---|---|
| System | Inoculum (cn/mL) | (cn/mL) |
| NTS-2.2 | 1.8 × 10⁵ | 6.8 × 10⁵-1.6 × 10⁷ |
| NTS-2.3 | 1.8 × 10⁵ | 1.3 × 10⁶-1.6 × 10⁷ |

These output solutions with the additional enrichment/retention of one or more of the target isolates, are expected to improve N content in plants measured by 1. Arabidopsis nitrogen stress relief, using only 10 mM ammonium salts, 2. measuring N nutrient levels in plant leaves after treatment with NTS solutions in greenhouse and outdoor pot assays, 3. ARA to evaluate nitrogen fixation capacity of plant roots and stems after treatment with NTS solutions, 4. evaluating recruitment of other beneficial microbes from the rhizosphere, and 5. performing a 15N labelled study to quantify the contribution of N₂ fixation after treatment when compared to a control.

### C. NTS-3.0 System

This is a production-scale version based on the NTS-2.3 system. The line is a continued serialized system. The system is a fluidized bed-based system that utilizes PST WB or PwST WB at a 1:3 floc: supernatant ratio as the microbial inoculum. This specific system comprises five reactors that use a down-flow orientation of the hydraulic re-circulation within each reactor and that are followed by a clarifier container from which the by products are recovered, (1) the floc that is separated by gravity and (2) the supernatant that is collected as the main product output of the system. Floc that is recovered from the clarifier is returned upstream of the system to Reactor 1. The addition of the PST WB or PwST WB is added to the hydraulic tank (rather than to R1) of the system at the same rate as the NTS-2.3 line, based on the hydraulic retention time of the system.

In NTS-3.0, glucose and ammonium sulfate are added to Reactor 1 to maintain a concentration range of 0.2-2.0% w/v and 0.02-0.2% w/v, respectively, based on the span of the hydraulic retention time of the system. Malate is also added to Reactor 1 at a concentration range of 0.2-2.0% w/v based on the span of the hydraulic retention time of the system. A concentration range of 0.5-4.5% v/v of Whole Broth of a Water Based Phosphate Solubilization Technology, PwST WB (in a ratio of 1:3 floc: SPN) is added to the hydraulic source tank based on the span of the hydraulic retention time of the system. Soy flour is added to Reactor 1 at a concentration range of 0.2-3.0% w/v, based on the span of the hydraulic retention time of the system. Fluid from the top of the Reactor 1 is continuously flowed into Reactor 2 in a hydraulically balanced fashion. Fluid from the top of the Reactor 2 is continuously flowed into Reactor 3, and from Reactor 3 to 4, and from 4 to 5, in a hydraulically balanced fashion. In some cases, fluid from the top of a fifth reactor is flowed into a separation chamber (clarifier), where solids are allowed to settle. The supernatant (or base product) from the clarifier is continuously collected, and a portion of the floc at the bottom of the clarifier is returned to Reactor 1 at a rate of 2% v/v of the system total volume.

The hydraulic rate of flow through the NTS-3.0 line is about 1.14 L/min to maintain a retention time of 14 days and can be varied accordingly for a retention time from 7 to 21 days depending on production volume requirements with a hydraulic rate range between 0.75-2.25 L/min. The total volume of working fluid within all the reactors and clarifier of the NTS-3.0 system is maintained at 6,000 gallons. In the NTS-3.0 line, the hydraulic rate of flow through the bioreactor system may be 1.13 L/min to maintain a retention time of 14 days and can be varied accordingly for a retention time from 7 to 21 days depending on production volume requirements. Because of this, the amount of product that is produced per day can range from 285.71 to 857.14 gallons. The amount of product that is produced per week can range from 2,000 to 6,000 gallons.

The NTS-3.0 system is built based on the NTS-2.3 design and process for optimization at a larger scale. The scaled-up systems comprises a series of six 1,000 gallon reactor chambers (Reactor 1b, Reactor 2b, Reactor 3b, Reactor 4b, and/or Reactor 5b) with continuous flow of working fluid from one reactor to the next and ultimately to the clarifier container, which is a 1,000 gallon reactor. Initially, a water tank is connected to Reactor 1b and provides continuous flow of water to Reactor 1b at an electrical conductivity ranging from 250-450 microsiemens/centimeter (μS/cm). The water tank comprises water from a city water source. Reactor 1b is also coupled to a pH sensor connected to a pH controller to provide an automatic buffer addition to maintain the pH at 7.0±0.1. When the pH drops below the set point, the pump connected to the controller adds base (3M NaOH) until the pH is again within the desired threshold. The working fluid from each reactor is recirculated within each reactor from the bottom of the reactor back to just below the surface of the working solution to maintain a homogeneous environment for the fermentation. For example, a portion of the working fluid from Reactor 1b is recycled back into Reactor 1b. The six reactors of the NTS-3.0 systems are fluidized bed reactors, which lack the scaffolding. The reactors can comprise a distribution ring that is subsurface of the discharge volume, reducing the amount of surface disruption, and keeping the environment in the reactor anaerobic. The hydraulic rate of flow through these systems is 1.13 L/min to maintain a retention time of 14 days and is varied accordingly for a retention time from 7 to 21 days depending on production volume requirements. Fluid from the top of Reactor 1b continuously flows into Reactor 2b in a hydraulically balanced fashion. The “target isolates” input into the NTS-3.0 system are MS3907, MS3900, and/or MS4921. These target isolates are inoculated into Reactor 1b. In NTS-3.0, glucose and ammonium sulfate are added to Reactor 1 to maintain a concentration range of 0.2-2.0% w/v and 0.02-0.2% w/v, respectively, based on the span of the hydraulic retention time of the system. Malate is also added to Reactor 1b at a concentration range of 0.2-2.0% w/v based on the span of the hydraulic retention time of the system. A concentration range of 0.5-4.5% v/v of Whole Broth of a Phosphate Solubilization Technology, PST WB (in a ratio of 1:3 floc: supernatant (SPN)) is added to the hydraulic source tank based on the span of the hydraulic retention time of the system. Soy flour is added to Reactor 1b at a concentration range of 0.2-3.0% w/v, based on the span of the hydraulic retention time of the system. Fluid from the top of the Reactor 1 is continuously flowed into Reactor 2b in a hydraulically balanced fashion. Fluid from the top of the Reactor 2 is continuously flowed into Reactor 3b, and from Reactor 3b to 4b, and from 4b to 5b, in a hydraulically balanced fashion. In some cases, fluid from the top of a fifth reactor is flowed into a separation chamber (clarifier), where solids are allowed to settle. The supernatant (or base product) from the clarifier is continuously collected, and a portion of the floc at the bottom of the clarifier is returned to Reactor 1 at a rate of 2% v/v of the system total volume. The clarifier comprises a floc flight system (e.g., floc-folding flights) that can improve the concentration of target isolated in the base product. The floc returns back to the system as a 2% v/v rate per day. Additionally, solids management is maintained at 20-25% range in the system.

### D. NTS-4.0 System

NTS-4.0 is built based on the NTS-3.0 design and process for optimization at a larger scale. The scaled-up system potentially comprises a series of six 12,000 gallon reactor chambers (similarly including Reactor 1b, Reactor 2b, Reactor 3b, Reactor 4b, and/or Reactor 5b) with continuous flow of working fluid from one reactor to the next and ultimately to the clarifier container, which is a 10,000 gallon reactor. Initially, a water tank is connected to Reactor 1b and provides continuous flow of water to Reactor 1b at an electrical conductivity ranging from 250- 450 microsiemens/centimeter (μS/cm). The water tank comprises water from a city water source. Reactor 1b is also coupled to a pH sensor connected to a pH controller to provide an automatic buffer addition to maintain the pH at 7.0±0.1. When the pH drops below the set point, the pump connected to the controller adds base (3M NaOH) until the pH is again within the desired threshold. In some cases, the working fluid from each reactor is recirculated within each reactor from the bottom of the reactor back to just below the surface of the working solution to maintain a homogeneous environment for the fermentation. For example, a portion of the working fluid from reactor 1b is recycled back into Reactor 1b. The six reactors of the NTS-4.0 systems are fluidized bed reactors, which lack the scaffolding. In some cases, the reactors comprise a distribution ring that is subsurface of the discharge volume, reducing the amount of surface disruption, and keeping the environment in the reactor anaerobic. The hydraulic rate of flow through these systems is 11.27 L/min to maintain a retention time of 14 days and can be varied accordingly for a retention time from 7 to 21 days depending on production volume requirements hydraulic rate range between 7.5-22.5 L/min. The “target isolates” input into the NTS-4.0 system are MS3907, MS3900, and/or MS4921. These target isolates are inoculated into Reactor 1b. In NTS-4.0, glucose and ammonium sulfate are added to Reactor 1 to maintain a concentration range of 0.2-2.0% w/v and 0.02-0.2% w/v, respectively, based on the span of the hydraulic retention time of the system. Malate is also added to Reactor 1b at a concentration range of 0.2-2.0% w/v based on the span of the hydraulic retention time of the system. A concentration range of 0.5-4.5% v/v of Whole Broth of a Water Based Phosphate Solubilization Technology, PwST WB or PST WB (in a ratio of 1:3 floc: SPN) is added to the hydraulic source tank based on the span of the hydraulic retention time of the system. Soy flour is added to Reactor 1b at a concentration range of 0.2-3.0% w/v, based on the span of the hydraulic retention time of the system. Fluid from the top of the Reactor 1 is continuously flowed into Reactor 2 in a hydraulically balanced fashion. Fluid from the top of the Reactor 2 is continuously flowed into Reactor 3, and from Reactor 3 to 4, and from 4 to 5, in a hydraulically balanced fashion. In some cases, fluid from the top of a fifth reactor is flowed into a separation chamber (e.g., clarifier), where solids are allowed to settle. The supernatant (or base product) from the clarifier is continuously collected, and a portion of the floc at the bottom of the clarifier is returned to Reactor 1 at a rate of 2% v/v of the system total volume. The clarifier comprises a floc flight system (e.g., floc-folding flights) that improves the concentration of target isolated in the base product. The floc returns back to the system as a 2% v/v rate per day. Additionally, solids management is maintained at 20-25% range in the system. The production rate of product for the NTS-4.0 system is 4,285.71 gallons per day and 30,000 gallons per week based on the hydraulic retention time of the system. The system output has a range of 2,857.14-8,571.43 gallons per day based on the hydraulic retention time range of 7-21 days. The system output can have a range of 20,000-60,000 gallons per week based on the hydraulic retention time range of 7-21 days.

### Example 13: Rainout Shelter Experiment

The objective of this study was to evaluate one NTS reactor product candidate, to which were added MS3900 alone or with MS3900 and MS4921 at 10⁴ CFU/mL each, applied in-furrow at 18 and 36 μL/plant on corn for PGP and NUE efficacy. The was Dyna-grow AC250 hybrid corn and the growing medium was 1:1:1 Profile MVP® Turface-peat-Denton sandy loam soil mix.

Fertilizer for 80% grower standard practice was: MESZ (12-40-0-10S-1Zn) at 32.8 lbs., nitrogen at planting (2.79 g/pot) and at 65.6 lbs., nitrogen side-dress at V6 stage (4.66 g/pot) and Potash at 73 lbs./A (1.04 g/pot), UAN 32 at 40 lbs./A, and/or N side-dress (1.368 mL/pot). Fertilizer for 100% grower standard practice was: MESZ (12-40-0-10S-1Zn) at 41 lbs., nitrogen at planting (3.48 g/pot) and at 82 lbs., nitrogen side-dress at V6 stage (5.82 g/pot) and Potash at 91.25 lbs./A (1.3 g/pot). An additional application of UAN 32 at 50 lbs./A, and nitrogen side-dress (1.7 mL/pot) was applied at the Vt growth stage.

Irrigation was maintained at least 90% field capacity throughout the season. For pest control, granular imidacloprid (Marathon) was applied at planting and then use broad-spectrum foliar pest control products at labelled intervals through the season. Weed control was maintained within the rainout shelter.

TABLE 22
Treatment Conditions for Rainout Shelter Study

| | A.I. | ml | Stock solution |
|---|---|---|---|
| | μl/40 | volume/seed | for 20 replicates |
| Treatment | cm pot | hole | μl A.I./10 ml |
| UTC-100% GSP | 0 | 0.5 | 0 |
| UTC-80% GSP | 0 | 0.5 | 0 |
| NTS-1.4 + MS3900-80% GSP-1 qt./A. | 18 | 0.5 | 360 |
| NTS-1.4 + MS3900 + MS4921-80% GSP-1qt./A | 18 | 0.5 | 360 |
| NTS-1.4 + MS3900-80% GSP-2 qt./A | 36 | 0.5 | 720 |
| NTS-1.4 + MS3900 + MS4921-80% GSP-2qt./A | 36 | 0.5 | 720 |

Corn was planted on Day 1 and fertilizer was applied on Day 4. Side-dress UAN32 fertilizer was next applied on Day 45 at the V6 growth stage. Corn growth stages are characterized by a V followed by a number, identifying the number leaf to emerge. UAN fertilizer was applied for the final time on Day 81 at the Vt growth stage. The Vt growth stage is approximately 2 to 3 days prior to silk appearing and when the last branch of the tassel is visible. A randomized complete block design was used with trade 10 gallon pots, at 40 cm diameter tops. There was one plant per pot and 20 replicates in the study.

120 pots with 40 cm diameter were filled with the growing medium. Three corn seeds were planted in each pot 2 in. deep. 0.5 ml of treatment solution was pipetted over the seed and the seed was covered with growing medium. Starter fertilizer (MESZ 12-40-10S-1Zn) was applied 2 inches away from the seed and 2 inches deep. The fertilizer was then watered in thoroughly. Next, MESZ fertilizer was side-dressed at the V6 growth stage. Soil plant analysis development (SPAD) measurements were taken from the youngest, fully-expanded leaf weekly throughout the season. Licor®-600 PF porometer and fluorometer readings were taken 3 times during the season. Ear leaves were collected at the beginning of the silking stage. Bulk four ear leaf samples from four plants of each treatment were taken to make five replicates for nutrient analysis. Plant height was evaluated at vegetative and reproductive stage (distance from the ground to the highest leaf collar/arch or the top of the tassel). Stem diameter was measured at harvest. At harvest, the following measurements were taken: grain yield (at 15.5% grain moisture), grain nutrient analysis, harvest index (grain/above-ground biomass), ear length, and ear weight. The data of leaf chlorophyll contents, plant physiological traits, corn tillers, plant heights, stem diameter, and dry shoot biomass were analyzed with JMP 16 software (SAS Institute, Cary, NC, USA) using the Fit model procedure at the p<0.1 level of significance.

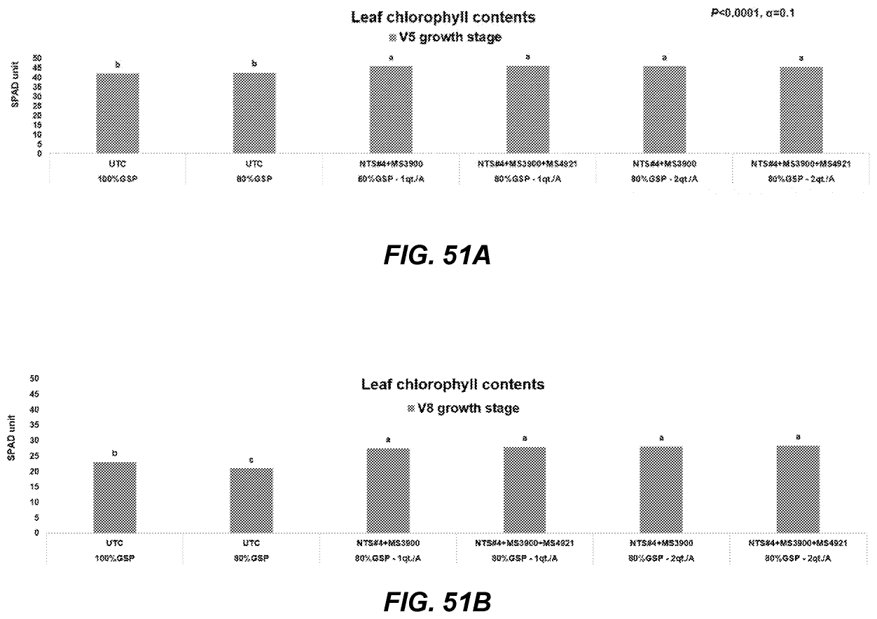

The leaf chlorophyll contents were measured from a fully expanded leaf at V5 and V8 growth stages using a Chlorophyll Meter, SPAD (Soil Plant Analysis Development-502, Konica Minolta, Tokyo, Japan). Three different positions on the leaf were measured and their average was recorded for the leaf chlorophyll contents. FIGS. 51A-51B show corn leaf chlorophyll contents at two growth stages. FIG. 51A shows leaf chlorophyll contents at V5 growth stage of corn (five leaves with collars) and FIG. 51B shows leaf chlorophyll contents at V8 growth stage of corn (eight leaves with collars). All NTS treated plants showed significantly increased leaf chlorophyll contents using both application rates (1 qt./A and/or 2 qt./A) and were significantly higher than the 100% GSP control.

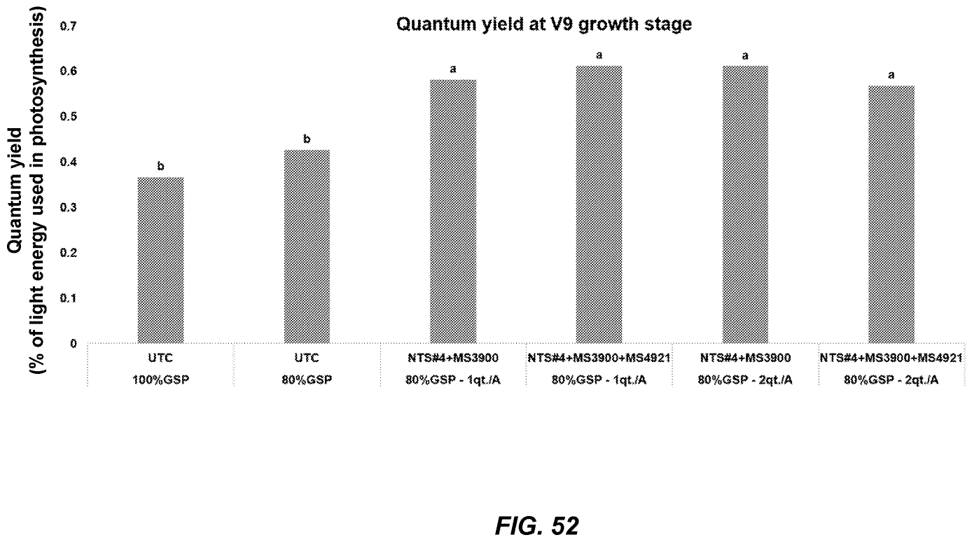

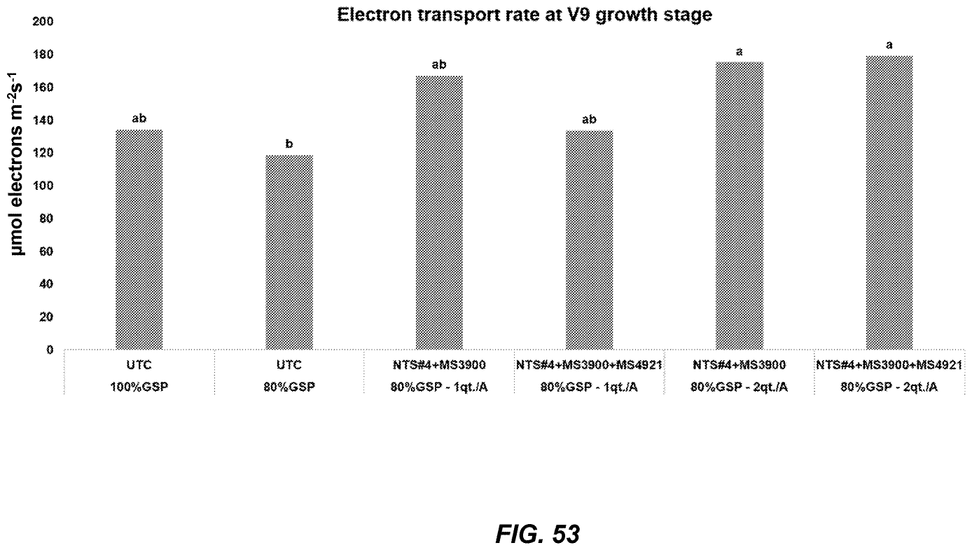

Plant physiological traits such as the stomatal conductance, transpiration rate, chlorophyll a fluorescence/quantum yield (QY), and electron transport rate (ETR) were measured using a LICOR 600 Porometer/Fluorometer Portable Photosynthesis System (Li-Cor, Inc. Lincoln, NE, USA) and irrigated the pots up to 90% soil moisture capacity. FIG. 52 shows the quantum yield (as percentage of light energy used in photosynthesis) at different growth stages. NTS treatments using either or both application rates (e.g., 1 qt./A and/or 2 qt./A) and with either one or two added isolates showed increased photosystem light capture efficiency. FIG. 53 shows the electron transport rate (i.e., photosynthesis capacity to assimilate carbon, μmoles e⁻/m₂/s) at different growth stages. NTS treatment conditions with either or both application rates (e.g., 1 qt./A and/or 2 qt./A) and one or two added isolates showed various photosystem energy transfer to ATP production results. The NTS treatments at 2 qt./A showed the greatest electron transport rate.

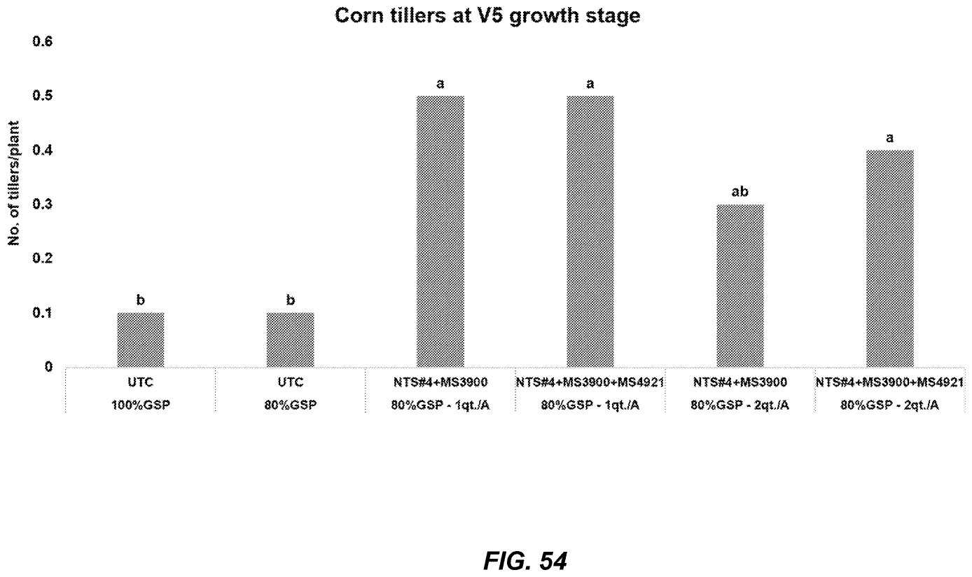

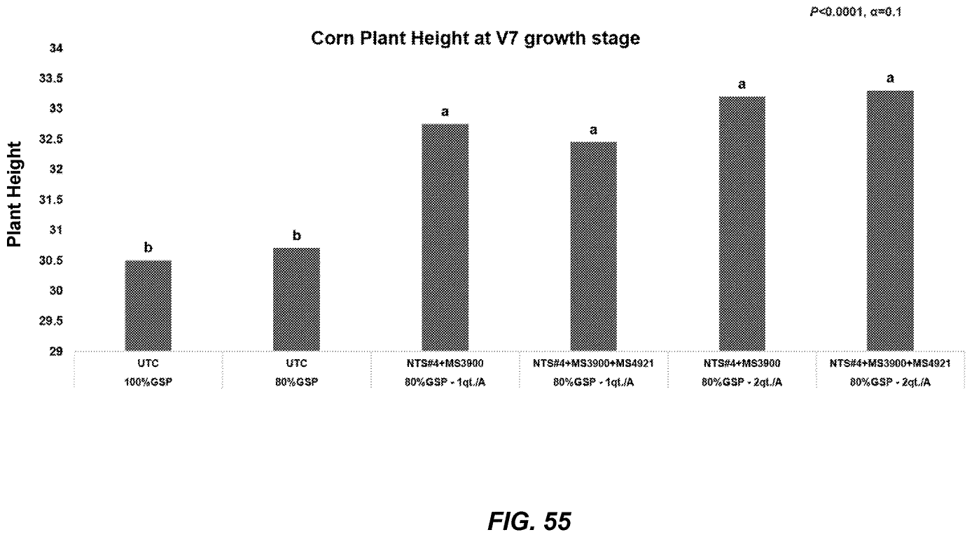

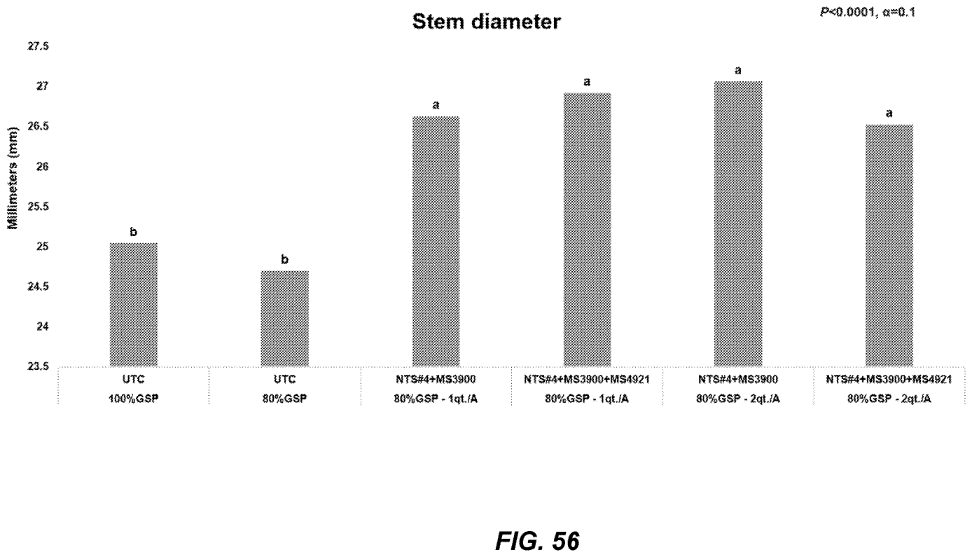

The corn tillers were counted at vegetative 5 (V5) growth stage for each treatment. The plant heights were measured in from the soil to the leaf collar number three at V7 growth stage. The stem diameters were measured at V8 growth stage. The plant heights and stem diameters were measured in centimeters and millimeters. FIG. 54 shows number of corn tillers per plant at the V5 growth stage. The NTS treatment conditions at the 1 qt./A in-furrow application rate promoted greater tiller production (e.g., NTS-1.4 with MS3900, NTS-1.4 with MS3900 and MS4921). This may be an indication of better overall plant health and biomass production potential. FIG. 55 shows corn plant height across treatment conditions. The NTS-treated plants showed significantly increased corn plant height using either or both application rates and one or two added isolates compared to corn height from untreated control (UTC) conditions. FIG. 56 shows the stem diameter across treatment conditions. All NTS-treated plants showed increased stem diameter using either or both application rates (e.g., 1 qt./A and/or 2 qt./A) and one or two spiked isolates compared to stem diameter from UTC conditions. There were no differences among NTS treatment conditions.

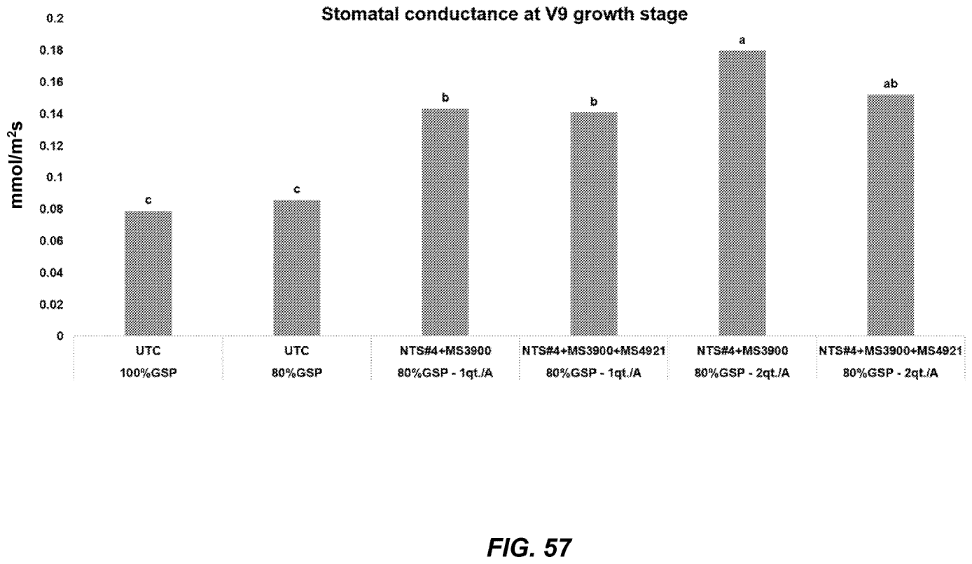

FIG. 57 shows the level of stomatal conductance (i.e., the rate of CO₂ and H₂O gas exchange, mmol H20/m2/s) across treatment conditions. Across NTS-treated plants, those treated at the 2 qt./A application rate showed improved gas exchange compared to that from the 1 qt./A NTS-treated plants. All NTS-treated plant conditions showed greater stomatal conductance compared to that from UTC conditions.

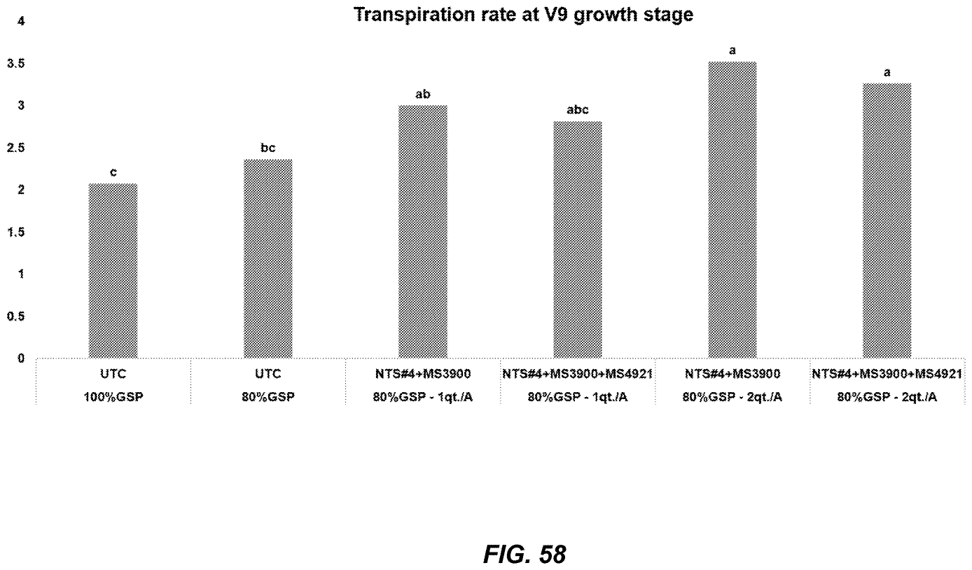

FIG. 58 shows the transpiration rate (i.e., efficiency of water movement into and through a plant, mmol H20/m2/s) across treatment conditions. Across NTS-treated plants, those treated at the 2 qt./A application rate showed superior transpiration rates compared to those from the 1 qt./A NTS-treated plants. This trend is similar to the gas exchange results of FIG. 17. All NTS-treated plant conditions showed greater transpiration rates compared to those from UTC conditions.

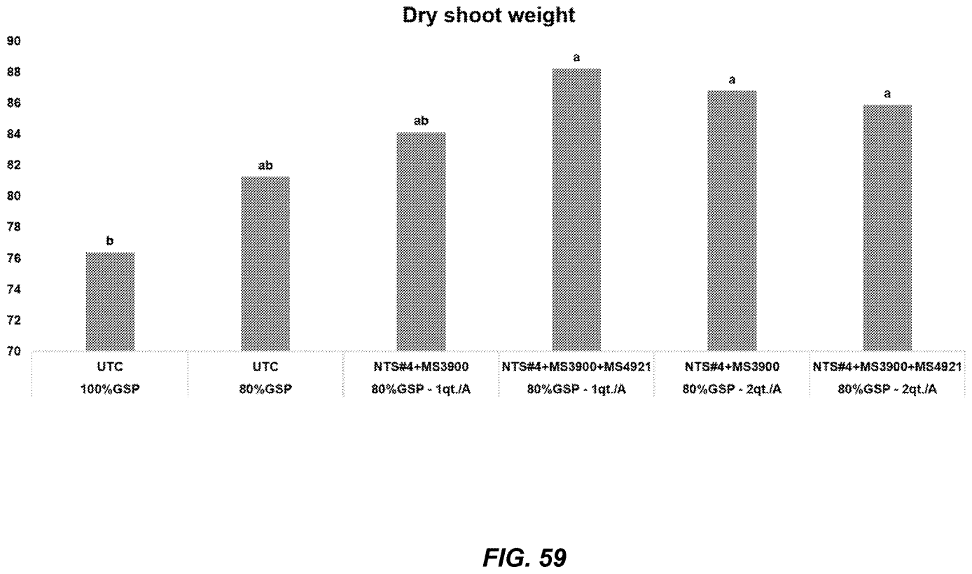

FIG. 59 depicts the dry shoot weight of corn plants (e.g., corn dry biomass) across treatment conditions. The NTS-treated plants showed increased shoot dry biomass compared to that from UTC conditions.

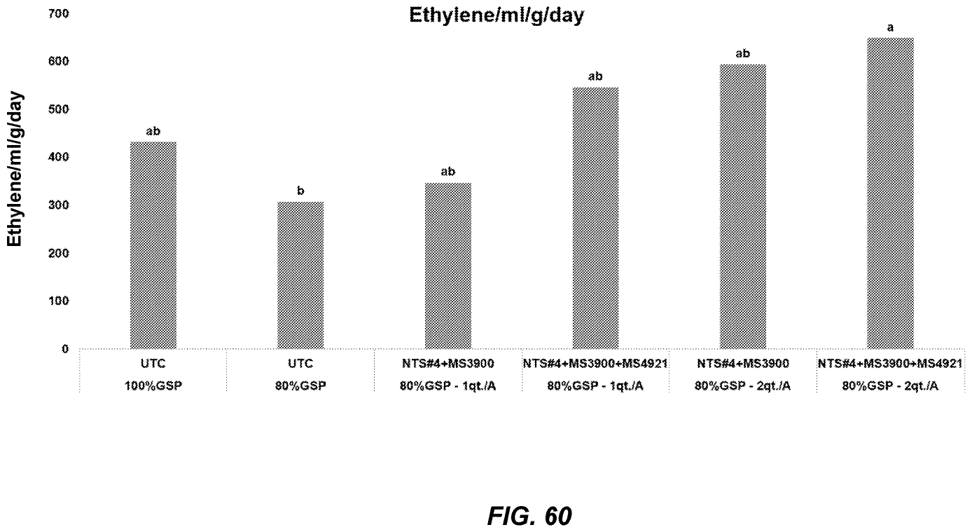

FIG. 60 shows nitrogenase activity (acetylene reduction measures) across treatment conditions (e.g., corn root acetylene reduction assay (ARA)). Acetylene reduction potentials of roots were measured as a proxy to nitrogenase activity. The NTS treatment condition with 2 qt./A application rate with both isolates (e.g., NTS-1.4 with MS3900 and MS4921) showed significantly increased nitrogen-fixing potential.

### Example 14: Optimization of NTS process design

### Determination of Aeration and WB Ratio

A shake flask study was designed to understand process environment optimization for target isolate MS3907. The study looked at both aerobic (high aeration) and anaerobic (low aeration) fermentations. The shake flasks were set for seven days shaking at 200 rpm for aerobic shake flasks and both 100 and 50 rpm for anaerobic at 30° C. Two-hundred- and fifty-mL flasks were used and the total working volume for aerobic conditions were 100 mL and 200 mL for anaerobic. The main hydraulic source for these experiments was water with an electrical conductivity range of 250-450 μS/cm. A microbial inoculum used for enrichment of nitrogen use efficiency bacteria and metabolites, PST WB at different ratios of floc: supernatant were used and applied at a v/v rate. Spore preps of target isolate MS3907 were used at a 2% v/v inoculum rate. Additional inputs such as soy flour, glucose, and ammonium sulfate were incorporated at 1.5%, 1.0%, and 0.1% v/v rate, respectively. The experiment looked at the survivability of the target isolate MS3907 as the sole target isolate in the shake flask or in combination with MS3900 in the shake flask when combined with the PST WB mixed consortium. Table 23 shows a list of treatments.

TABLE 23
List of treatments for shake flask study

| Internal | | | PST WB ratio |
|---|---|---|---|
| Code | Aeration | RPM | (Floc:SPN) |
| A | Anaerobic | 50 | 1:1 |
| B | Anaerobic | 50 | 2:1 |
| C | Anaerobic | 50 | 1:9 |
| D | Anaerobic | 50 | 0:1 |
| E | Anaerobic | 100 | 1:1 |
| F | Anaerobic | 100 | 2:1 |
| G | Anaerobic | 100 | 1:9 |
| H | Anaerobic | 100 | 0:1 |
| I | Aerobic | 200 | 1:1 |
| J | Aerobic | 200 | 2:1 |
| K | Aerobic | 200 | 1:9 |
| L | Aerobic | 200 | 0:1 |

After seven days, shake flask solutions were streaked out on quarter-strength Tryptic Soy Agar (Difco, TSA). Plates were incubated for 7 days at 30° C. Isolate MS3907 showed an improved recovery under anaerobic conditions (50 and 100 rpm) with a PST WB ratio of 0:1 Floc: SPN.

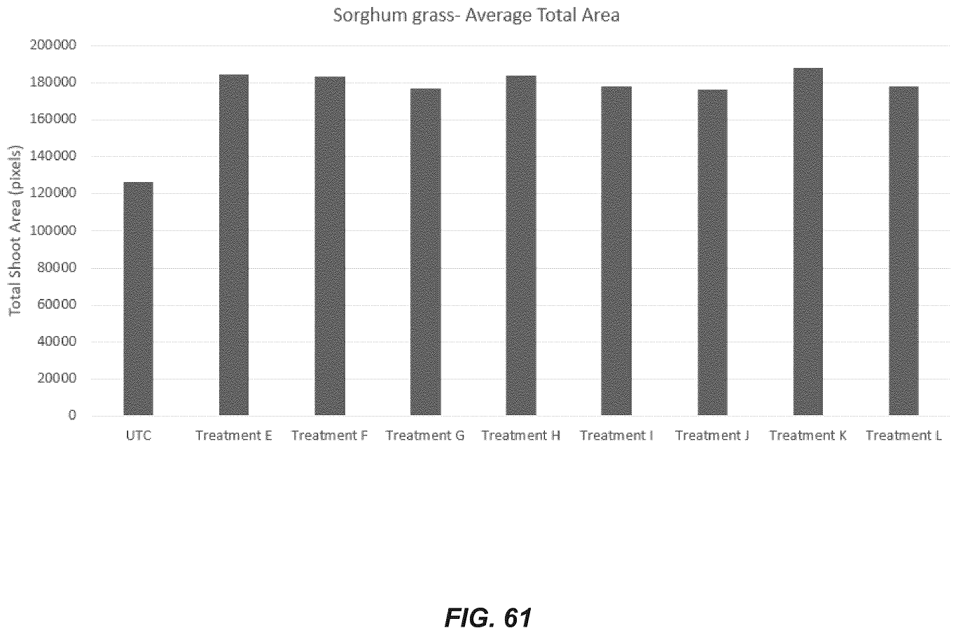

The shake flask solutions described above were tested in sorghum grass. A greenhouse rapid test was set up to evaluate the plant growth promotion of the shake flask solutions under no N-fertilization. The growing medium for this experiment was isolite: Berger BM6 potting mix at a 9:1 ratio v/v into a 4×4×3.75″ size pots. A total of sixteen seeds were sown per pot with thinning to the lowest germination number after one week. Seeds were placed over the surface of the potting mix and a total of 10 mL per treatment were applied to the top of the seeds. The seeds and application were then covered with 100 mL of potting mix. Experiment took place in the greenhouse and were watered from bottom up from a saucer. Within the first week (5-7 days) pots were thinned to a minimum 9 per pot. Plants were then fertilized with 100 mL containing 0.0402 grams of Hoagland's without N and 0.2 grams of KH2PO4. The plants were then imaged after 14 days. A total of 10 replicates per treatment were used. Imaging data was used as the main metric to evaluate plant growth promotion. After 14 days, all treatments showed significantly more leaf area compared to that from the untreated control (FIG. 61).

Treatments were then analyzed for trends. Total shoot area performance for the shake flasks were grouped by the type of aeration. Shake flasks E-H solutions were generated under anaerobic conditions (e.g., low aeration) while shake flasks I-L were generated under aerobic conditions (e.g., high aeration). The low aeration grouped shake flasks showed greater average pixel shoot area compared to that from high aeration grouped shake flasks (FIG. 62).

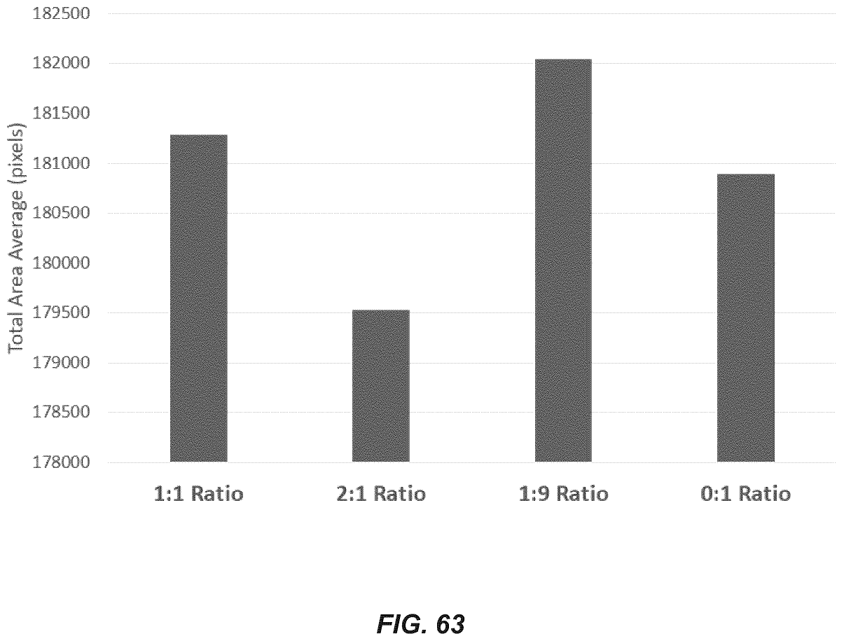

Shake flask treatments were also grouped based on the ratio of PST WB used under both aerobic and anaerobic conditions. The ratios of PST WB were 1:1 PST WB, 2:1 PST WB, 1:9 PST WB, and 0:1 PST WB. The treatment with 1:9 PST WB ratio showed numerically higher plant growth promotion over other treatments (FIG. 63).

Target isolate MS3907 was able to be recovered from anaerobic shake flasks at both 50 and 100 rpm with a PST WB of 0:1 floc: SPN. When the shake flasks solutions were looked at for plant growth promotion in sorghum grass, all shake flasks at either fermentation aeration or

PST WB inoculum ratio performed significantly better than the untreated control. When treatments were grouped by fermentation aeration, low aeration solutions trended higher in more shoot biomass pixels than high aeration treatments. When shake flask treatments were grouped by PST WB ratio, all treatments performed similar with treatments with 1:9 PST WB ratio had numerically higher plant growth promotion over other treatments. Based on the information, a system with low aeration (anaerobic) and a higher proportion of SPN in the WB, will provide favorable conditions when applied to a serialized system to retain and probably enrich target isolate MS3907 as well as to generate an output solution with NUE and PGP properties. Determination of PST Component Utilization

A parallel shake flask experiment was set up looking at different components of the PST as microbial inoculums for NUE enrichment. PST WB (1:1) floc: SPN was used as well as PST SPN alone and incorporated in a similar manner to the previous shake flask experiment at 1.0% v/v. The study looked at both aerobic and anaerobic fermentations. The shake flasks were set for seven days shaking at 200 rpm for aerobic shake flasks and both 100 and 50 rpm for anaerobic at 30° C. Two-hundred- and fifty-mL flasks were used and the total working volume for aerobic conditions were 100 mL and 200 mL for anaerobic. The main hydraulic source for these experiments was water with a conductivity range of 250-450 μS/cm. A microbial inoculum used for enrichment of nitrogen use efficiency bacteria and metabolites, PST WB at different ratios of floc: supernatant were used and applied at a v/v rate. Spore preps of target isolate MS3907 were used at a 1% v/v inoculum rate. Additional inputs such as soy flour, glucose, and ammonium sulfate were incorporated at 1.5%, 1.0%, and 0.1% v/v rate, respectively.

Anaerobic conditions favored an enrichment of the nifH component with both the WB (1:1) component as well as the SPN only. When PST SPN only was used as the main microbial inoculum, there was an increase in nifH gene detected per ng DNA when the target isolates MS3907 and MS3900 were not present.

The purpose of the experiment was to understand the optimization of the conditions and environments that could select for enrichment of nitrogen use efficiency (NUE). Under anaerobic conditions, there is a clear enrichment of nifH per ng DNA when compared to the aerobic counterparts. When the solutions were tested for acetylene reduction, there was higher activity under anaerobic shake flask with PST SPN as the microbial inoculum. A reduction of the ratio of the WB (1:1) may be ideal for enrichment of NUE by increasing the SPN content in the microbial inoculum.

Characterization of NTS batch system and effects of batch system product on plant growth promotion

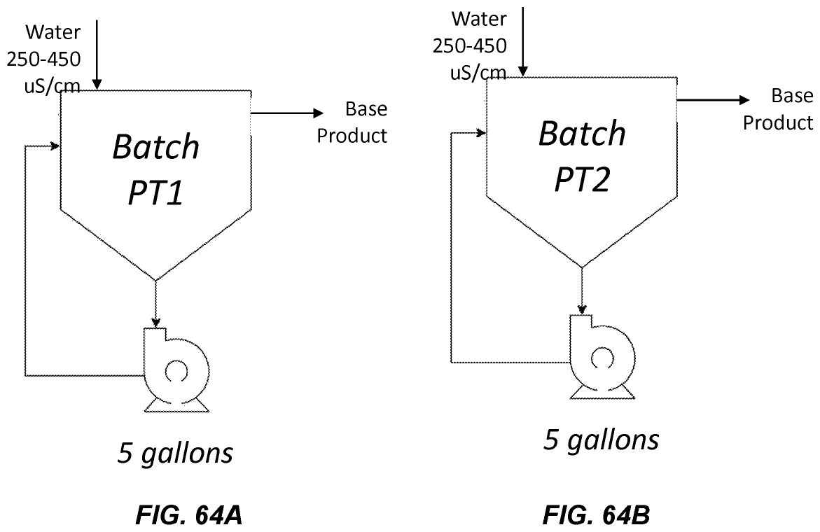

Two separate systems NTS-PT1 and NTS-PT2 (e.g., NTS batch systems) were developed for production of nitrogen use efficiency promoting products by digestion of organic feedstock. These two systems were down-flow systems and ran between 7-10 days. Each system included one 5-gallon reactor chamber. A water tank was connected to the system to provide continuous flow of water to the reactor at an electrical conductivity ranging from 250-450 microsiemens/centimeter (μS/cm). The water tank comprised water from a city water source and a reverse osmosis (RO) water source. The reactor of the NTS-PT1 and NTS-PT2 systems were fluidized bed reactors, which lacked scaffolding. In some cases, the working fluid in each reactor is recirculated from the bottom of the reactor and pumped back to just below the surface of the same reactor to maintain homogeneous conditions within the working solutions. Diagrams of the NTS-PT1 and NTS-PT2 systems are shown in FIGS. 64A and 64B.

In each of NTS-PT1 and NTS-PT2, glucose and ammonium sulfate were added to the batch system to maintain a concentration range of 0.2-2.0% w/v and 0.02-0.2% w/v, respectively. A concentration range of 0.5-4.5% v/v of PST WB (in a ratio of 1:1 floc: supernatant (SPN)) was also added NTS-PT2 and concentration range of 0.5-4.5% v/v of the PST SPN was added to the NTS-PT1. Soy flour was added to the batch system at a concentration range of 0.2-3.0% w/v.

The produced NTS-PT1 base products had a pH range of 4.0-8.0 during the batch process. The produced NTS-PT2 base products had a pH range of 3.8-8.0 during the batch process. No buffer and/or pH adjustment was implemented during the fermentation process. In the NTS-PT1 batch system, the dissolved oxygen range was 0.19-0.9 mg/L during the batch process. In the NTS-PT1 batch system, the dissolved oxygen range was 0.12-0.7 mg/L during the batch process.

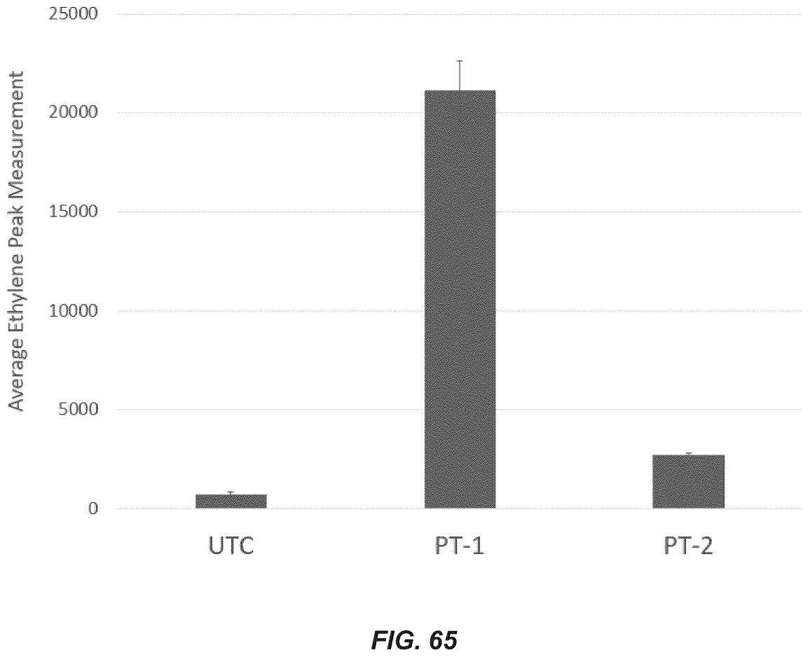

Acetylene reduction assays were conducted from the output solutions of the NTS-PT1 and NTS-PT2 systems. Methods were similar as to those in Example 11. The products of both systems showed N-fixing capacity (FIG. 65).

A rapid greenhouse assay experiment was set up to evaluate the efficacy of the NTS-Batch systems PT-1 and PT-2 in a nitrogen-free fertilizer system. Corn was planted in 4×4 inch pots with a soil medium consisting of 1:1:1 mixture of Turface: sifted Whitesboro (local town) soil; and ProMix. Three seeds were planted initially and later thinned to one seed following treatment. The treatments were diluted to 16 ml/L or 32 ml/L. The diluted treatments were added at the rate of 0.5 ml per pot directly over the seeds prior to covering them with soil and this resulted in rates of 2 qt/acre and 4 qt/acre. For fertilization, 4.02 g/L N-free Hoagland's+2 g/L KH2PO4 was delivered at a rate of 100 ml per pot at planting time. Done only once during the experiment. Pots were watered from the top of the pot when needed. The experiment ran for 14 days and leaf area using a plant imaging system was used as the main metric for promotion of plant growth over the UTC. Images were taken at 10 and 14 days of the experiment and the NTS-PT1 and NTS-PT2 were evaluated at rates of 2 qt/acre and 4 qt/acre.

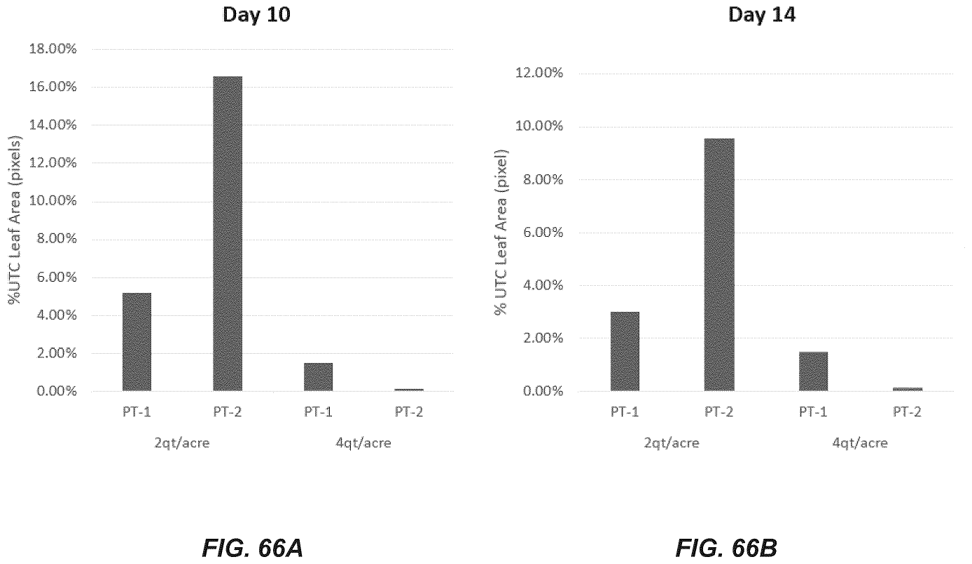

Results showed both batch systems having greater leaf area in both imaging times over the untreated control. Both systems performed better at the 2 qt/acre rate at the Day 10 and Day 14 timepoints (FIGS. 66A-66B). NTS-PT2 at 2 qt/A showed the greatest leaf area compared to that of NTS-PT1 at 2 qt/A.

### Characterization of Hydraulic Retention Time and Number of Reactors in System

The initial design of the NTS-1.0 lines consisted of three reactors: R1, R2, and the Clarifier. The initial hydraulic retention time of the systems was 7 days. The systems were tested in Arabidopsis for plant growth promotion. NTS-1.0 base product solutions were tested in Arabidopsis for plant growth promoting ability, using average leaf area (in cm2) as the primary metric. All systems were tested under two rates, 0.05% and 0.2% v/v. Arabidopsis thaliana (genotype Col-0) seedlings were germinated and grown for 7 days on ½ strength MS media (Murashige & Skoog Basal Medium with Vitamins, M519) plates in the growth chamber before transplanting into rockwool cubes for plant growth assays. Rockwool cubes were used as an inert substrate for growing Arabidopsis in a hydroponic-like system. The rockwool cubes are fitted into a 6-well tray and each treatment consisted of three replicate well trays. Nine, 1 L ½ strength MS media (Murashige & Skoog Basal Medium with Vitamins, M519) solutions were made. The test substance was then added at the rate given below in Table 13, and the pH of the solution was raised to 5.7. Forty mL of each 1 L treatment solution was dispensed to each rockwool cube. Then, each wetted cube received one Arabidopsis seedling. Arabidopsis plants grew on an LED light cart for 12 days. Leaf area was determined using the ImageJ software package. All the data collected were put through JMP for ANOVA analysis.

TABLE 24
Components of each test solution

| | | |
|---|---|---|
| 1. | ½MS Media+ | UTC |
| 2. | ½MS Media+ | 0.05% NTS-1.1 Base Product |
| | | Solution (v/v) (0.5 mL/L) |
| 3. | ½MS Media+ | 0.05% NTS-1.2 Base Product |
| | | Solution (v/v) (0.5 mL/L) |
| 4. | ½MS Media+ | 0.05% NTS-1.3 Base Product |
| | | Solution (v/v) (0.5 mL/L) |
| 5. | ½MS Media+ | 0.05% NTS-1.4 Base Product |
| | | Solution (v/v) (0.5 mL/L) |
| 6. | ½MS Media+ | 0.2% NTS-1.1 Base Product |
| | | Solution (v/v) (2.0 mL/L) |
| 7. | ½MS Media+ | 0.2% NTS-1.2 Base Product |
| | | Solution (v/v) (2.0 mL/L) |
| 8. | ½MS Media+ | 0.2% NTS-1.3 Base Product |
| | | Solution (v/v) (2.0 mL/L) |
| 9. | ½MS Media+ | 0.2% NTS-1.4 Base Product |
| | | Solution (v/v) (2.0 mL/L) |

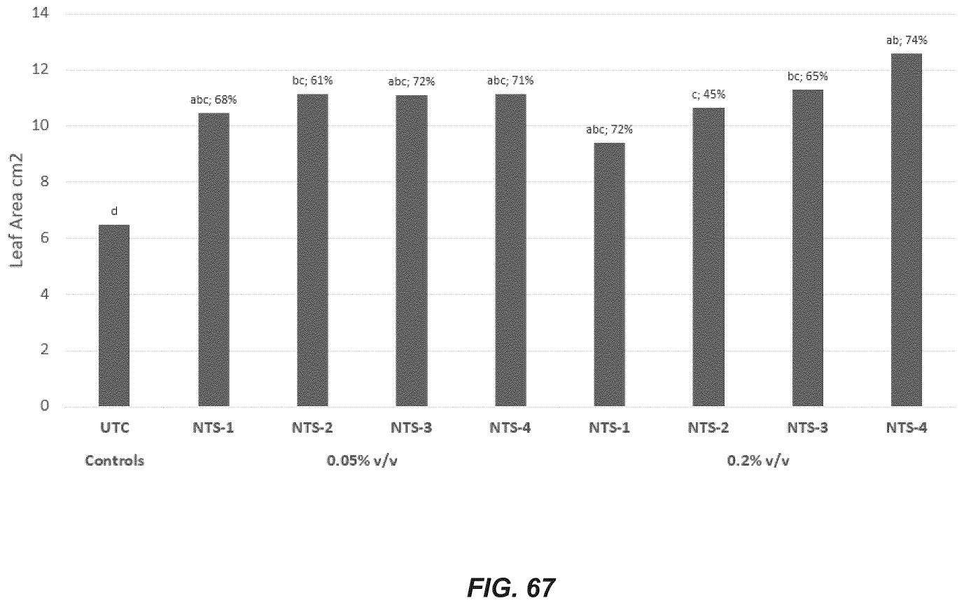

All base product solutions across all systems showed consistent PGP activity over the UTC control (FIG. 67). All treatments showed numerically greater average leaf area measurement from the UTC with all having significant plant growth promotion (p=0.0008).

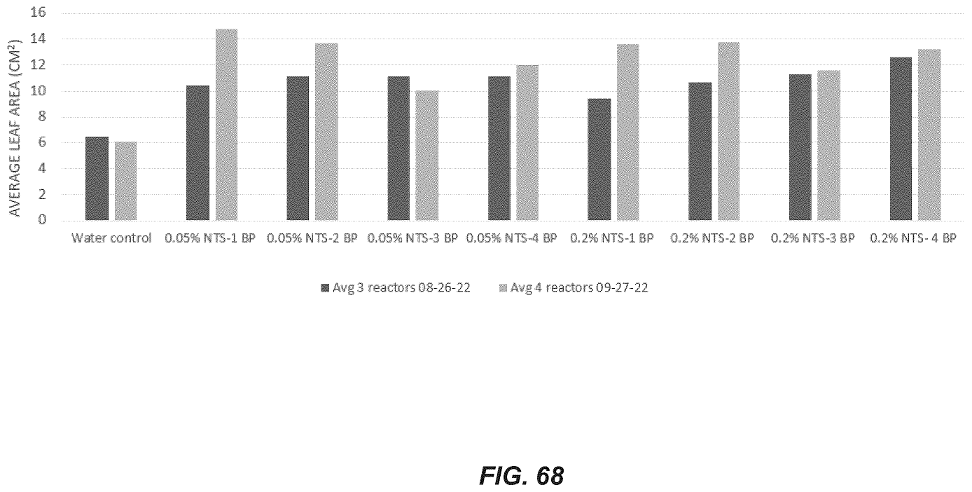

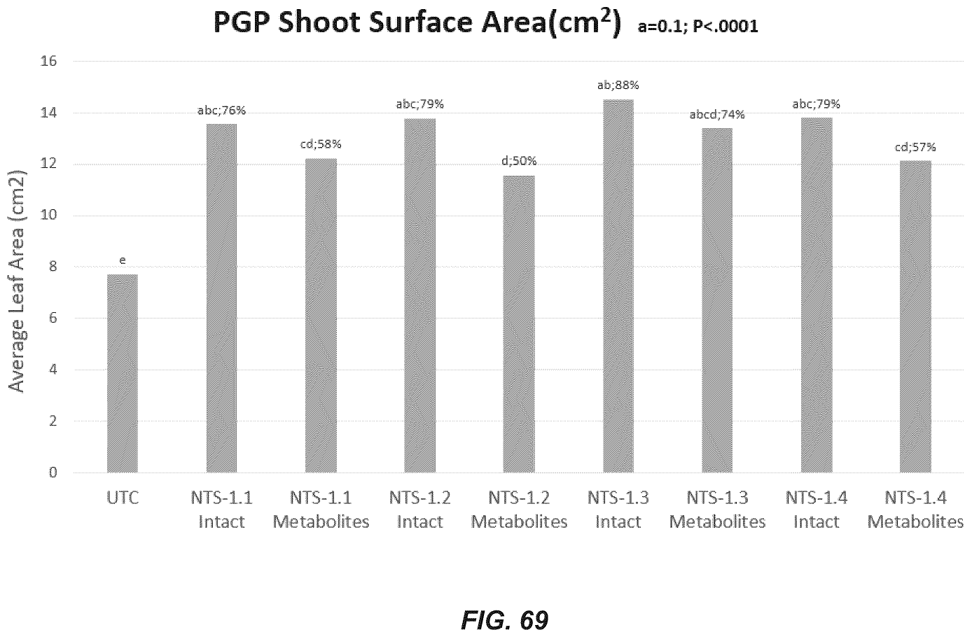

Each of the four lines received an additional reactor (R3) in the same volume as R1 and R2. This extended the hydraulic retention time of the system by about 42.86% (10 days). The solution was then tested again in Arabidopsis for plant growth promotion in same manner as the previous test. The additional reactor and extended hydraulic retention time showed improvements in plant growth promotion in most lines (FIG. 68). There was a general increase of performance in the output solutions when the retention time of the system was extended by about 42.86%.

### Example 15: Effects of intact and NTS metabolites on plant growth promotion

A version of the product that was filter sterilized to remove the bacteria from the system product also promoted plant growth in experiments with Arabidopsis.

NTS-1.0 base product solutions were tested in Arabidopsis for plant growth promoting ability, using average leaf area (in cm²) as the primary metric. All systems were tested under 0.8% v/v and intact and filter-sterilized looking at the metabolites of the solutions. Solutions were filter-sterilized using a 0.22-micron filter. Arabidopsis thaliana (genotype Col-0) seedlings were germinated and grown for 7 days on ½ strength MS media (Murashige & Skoog Basal Medium with Vitamins, M519) plates in the growth chamber before transplanting into rockwool cubes for plant growth assays. Rockwool cubes were used as an inert substrate for growing Arabidopsis in a hydroponic-like system. The rockwool cubes were fitted into a 6-well tray and each treatment consisted of three replicate well trays. Nine, 1 L ½ strength MS media (Murashige & Skoog Basal Medium with Vitamins, M519) solutions were made. The test substance was then added at the rate given below in Table 25, and the pH of the solution was raised to 5.7. Forty mL of each 1 L treatment solution was dispensed to each rockwool cube. Then, each wetted cube received one Arabidopsis seedling. Arabidopsis plants grew on an LED light cart for 12 days. Leaf area was determined using the ImageJ software package. All the data collected were put through JMP for ANOVA analysis. All treatments showed numerically greater average leaf area measurement from the UTC with NTS-1 having the most significant plant growth promotion (p<0.0001) (FIG. 86). All base product solutions across all systems showed consistent PGP activity over the UTC control. The results demonstrated that intact solutions and metabolites alone promoted higher leaf area in Arabidopsis.

TABLE 25
Treatment conditions for Arabidopsis metabolite study

| | | |
|---|---|---|
| 1. | ½MS Media+ | UTC |
| 2. | ½MS Media+ | 0.8% NTS-1.1 Base Product |
| | | Solution Intact (v/v) (8 mL/L) |
| 3. | ½MS Media+ | 0.8% NTS-1.1 Base Product Solution |
| | | Metabolites (v/v) (8 mL/L) |
| 4. | ½MS Media+ | 0.8% NTS-1.2 Base Product Solution |
| | | Intact (v/v) (8 mL/L) |
| 5. | ½MS Media+ | 0.8% NTS-1.2 Base Product Solution |
| | | Metabolites (v/v) (8 mL/L) |
| 6. | ½MS Media+ | 0.8% NTS-1.3 Base Product Solution |
| | | Intact (v/v) (8 mL/L) |
| 7. | ½MS Media+ | 0.8% NTS-1.3 Base Product Solution |
| | | Metabolites (v/v) (8 mL/L) |
| 8. | ½MS Media+ | 0.8% NTS-1.4 Base Product Solution |
| | | Intact (v/v) (8 mL/L) |
| 9. | ½MS Media+ | 0.8% NTS-1.4 Base Product Solution |
| | | Metabolites (v/v) (8 mL/L) |

### Example 16: Plant Growth Promotion Capacity in NTS Systems as Intact Solution or as Metabolites

Plant growth promotion was tested in Arabidopsis for the NTS 2.0 lines as both intact and filter-sterilized (F/S; e.g., metabolite) solutions. Arabidopsis thaliana (genotype Col-0) seedlings were germinated and grown for 7 days on ½ strength MS media (Murashige & Skoog Basal Medium with Vitamins, M519) plates in the growth chamber before transplanting into rockwool cubes for plant growth assays. Rockwool was used as an inert substrate for growing Arabidopsis in a hydroponic-like system. The rockwool cubes may be fitted into a 6-well tray and each treatment consisted of three replicate well trays. Thirteen, 1 L ½ strength MS media (Murashige & Skoog Basal Medium with Vitamins, M519) solutions were made. The test substance was then added at the rate as shown below in Table 26 and the pH of the solution was raised to 5.7. Forty mL of each 1 L treatment solution was dispensed to each rockwool cube. Each wetted cube then received one Arabidopsis seedling.

TABLE 26
Treatment conditions for testing plates

| | | | Application | Type of |
|---|---|---|---|---|
| | | Treatment | Rate (v/v) | Solution |
| 1 | ½MS Media+ | UTC | | |
| 2 | ½MS Media+ | NTS-2.2 BP | 0.05% | Intact |
| 3 | ½MS Media+ | | | Metabolites |
| 4 | ½MS Media+ | | 0.2% | Intact |
| 5 | ½MS Media+ | | | Metabolites |
| 6 | ½MS Media+ | | 0.8% | Intact |
| 7 | ½MS Media+ | | | Metabolites |
| 8 | ½MS Media+ | NTS-2.3 BP | 0.05% | Intact |
| 9 | ½MS Media+ | | | Metabolites |
| 10 | ½MS Media+ | | 0.2% | Intact |
| 11 | ½MS Media+ | | | Metabolites |
| 12 | ½MS Media+ | | 0.8% | Intact |
| 13 | ½MS Media+ | | | Metabolites |

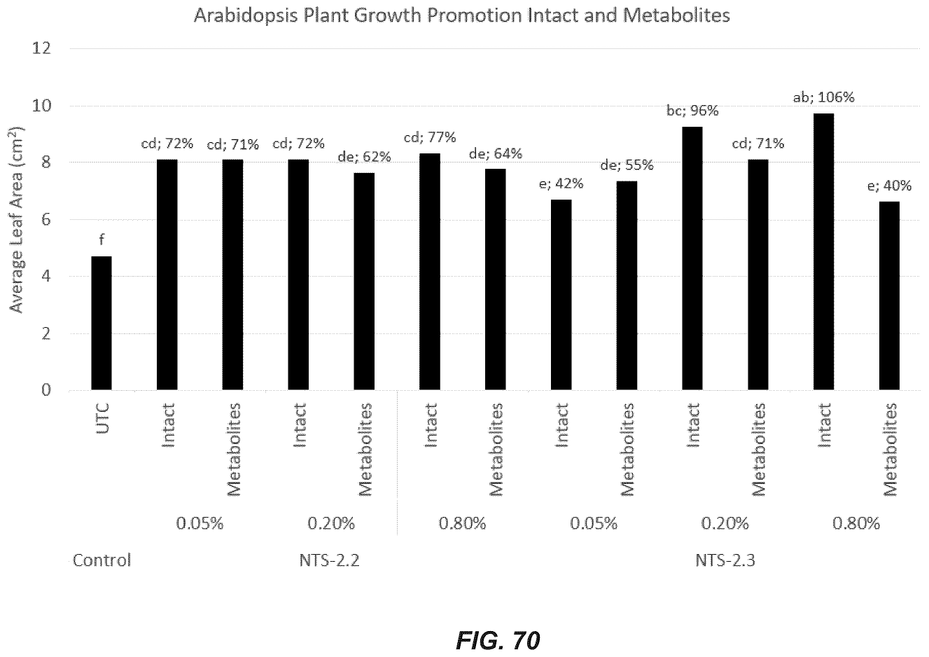

Arabidopsis plants grew on an LED light cart for 12 days. Leaf area was determined using the ImageJ software package. All the data collected were put through JMP for ANOVA analysis (a=0.1, p<0.0001). Results show that at all application rates, both NTS 2.0 lines have significantly improved the Arabidopsis leaf area over the untreated control when the solutions are intact (FIG. 70). Additionally, the metabolites produced along the process and that remained in the base product also had a significant effect on plant growth.

Plant growth promotion was also tested in Arabidopsis for the NTS 1.4 and NTS 1.5 systems as both intact and filter-sterilized (F/S; i.e., metabolite) solutions. The experimental methods were similar to those above for the NTS 2.0 systems. Seven 1 L ½ strength MS media (Murashige & Skoog Basal Medium with Vitamins, M519) solutions were made. The test substance was then added at the rate as shown below in Table 27, and the pH of the solution was raised to 5.7. Leaf area was determined using the ImageJ software package. All the data collected were put through JMP for ANOVA analysis with a=0.01 and p value=0.0025.

TABLE 27
Treatment conditions for testing plates

| | | |
|---|---|---|
| 1 | ½MS Media+ | UTC (untreated control) |
| 2 | ½MS Media+ | 0.2% NTS-1.5 BP (v/v) |
| 3 | ½MS Media+ | 0.2% NTS-1.5 BP + Isolates (v/v) |
| 4 | ½MS Media+ | 0.2% NTS-1.4 BP (v/v) |
| 5 | ½MS Media+ | 0.8% NTS-1.5 BP Metabolites (v/v) |
| 6 | ½MS Media+ | 0.8% NTS-1.5 BP + Isolates Metabolites (v/v) |
| 7 | ½MS Media+ | 0.8% NTS-1.4 BP Metabolites (v/v) |

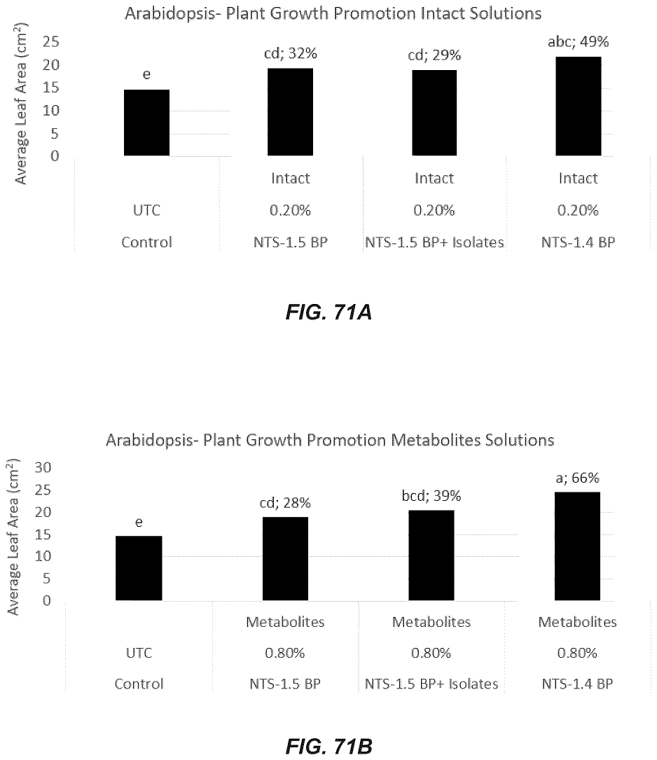

In this experiment NTS 1.5 (no isolates) was evaluated to compare the metabolites of the base product (BP) to the metabolites of the NTS-1.4 (inoculated with target isolates within the reactor system, MS3900 and MS3907), as well as NTS-1.5 base product with the target isolates spiked at the end to the final base product (NTS-1.5 BP+Isolates). When the intact solutions were evaluated for plant growth promotion in Arabidopsis, there was a numerical increase in average leaf area of plants treated with NTS-1.4 BP over the NTS-1.5 BP and NTS-1.5 BP+Isolates treatments (FIG. 71A). All treatments had significantly greater leaf area than the untreated control (UTC). The metabolites of these solutions tested at 0.8%, showed a greater effect in increase leaf area in Arabidopsis with the NTS-1.4 metabolites being significantly different with greater leaf area than the NTS-1.5 metabolite-treated plants (FIG. 71B). These results demonstrated that the metabolites generated in the process by the target isolates or under the presence of the target isolate had plant growth promotion properties.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1-183. (canceled)

184. A method, comprising:
(a) providing a bioreactor system comprising two or more containers arranged in a series, each of the two or more containers comprising a volume of a working fluid, wherein a first container of the two or more containers comprises an established population of a microbial strain having a plant growth promoting property; and
(b) operating the bioreactor system for a duration of time by:
(i) transferring into the first container an aqueous feedstock comprising a microbial consortium;
(ii) transferring a portion of the working fluid out of each of the two or more containers into either a subsequent container of the bioreactor system or a product outflow stream; and
(iii) collecting at least a portion of the product outflow stream as a biostimulant composition, wherein the biostimulant composition comprises an amount of the microbial strain;
wherein the biostimulant composition is configured to promote a plant growth property of a plant administered the biostimulant composition as compared to that of a plant not administered the biostimulant composition.

185. The method of claim 184, wherein the plant growth property comprises an ability to: promote uptake of a nutrient by one or more plants, increase bioavailability of a nutrient in soil, recruit microbes having the desired plant growth promoting property to root zones or other tissues of plants, stimulate plant growth, increase yield of a crop, increase shoot biomass, increase root biomass, increase deaminase activity, increase acid production, increase leaf area, increase chlorophyll content, increase heat tolerance, increase cold tolerance, increase drought tolerance, increase salt tolerance, increase total biomass, or any combination thereof.

186. The method of claim 184, wherein the established population of the microbial strain in (b) is incubated for a duration of time.

187. The method of claim 186, wherein the duration of time is at least 5 days.

188. The method of claim 186, wherein a concentration of the microbial strain in the aqueous feedstock of (b) (i) is at most 1×10² CFU/ml during the duration of time.

189. The method of claim 184, wherein the microbial strain in the first container is biologically distinct from the microbial consortium present in the aqueous feedstock.

190. The method of claim 184, wherein the established population of the microbial strain comprises a zinc solubilizing microbial strain.

191. The method of claim 190, wherein the zinc solubilizing microbial strain comprises Bacillus safensis, Bacillus amyloliquefaciens, Bacillus megaterium, Pseudomonas spp., Agrobacterium spp., Bacillus circulans, Burkholderia lata, Lysinibacillus spp., Burkholderia latens, Bacillus cereus, B. aryabhattai, Pseudomonas taiwenensis, Acinetobacter spp., Paenibacillus sonchi, Chitinophaga spp., Gluconobacter spp., Klebsiella spp., Serratia spp., Thiobacillus spp., or any combination thereof.

192. The method of claim 184, wherein the established population of the microbial strain comprises a phosphate solubilizing microbial strain.

193. The method of claim 192, wherein the phosphate solubilizing microbial strain comprises Bacillus safensis, Bacillus amyloliquefaciens, Bacillus licheniformis, Bacillus megaterium, Commonas testosteroni, Enterobacter cloacae, Citrabacter freundii, Pseudomonas spp., Agrobacterium spp., Bacillus circulans, or any combination thereof.

194. The method of claim 184, wherein the established population of the microbial strain comprises a nitrogen use efficiency-promoting microbial strain.

195. The method of claim 194, wherein the nitrogen use efficiency-promoting microbial strain comprises Kosakonia sacchari, Klebsiella variicola, Rahnella aquatilis, Kluyvera intermedia, Kosakonia pseusosacchari, Enterobacter spp., Achromobacter marplatensis, Azopirillum lipoferum, Microbacterium murale, Gluconobacter diazotrophicus, Methylobacterium symbioticum, Paenibacillus borealis, Bacillus megaterium (Priestia megaterium), Paenibacillus sonchi, or any combination thereof.

196. The method of claim 184, wherein the aqueous feedstock further comprises an organic material at least partially digestible by microbes present in at least one of the two or more containers.

197. The method of claim 196, wherein, before the transferring of (b) (i), the organic material is partially digested by microbes endogenous to the organic material.

198. The method of claim 184, wherein the working fluid further comprises an inorganic material.

199. The method of claim 198, wherein the inorganic material comprises rock phosphate or zinc oxide.

200. The method of claim 184, further comprising adding one or more carbon sources to at least one container of the two more containers of the bioreactor system.

201. The method of claim 184, further comprising adding one or more nitrogen sources to at least one container of the two or more containers of the bioreactor system.

202. The method of claim 184, wherein the bioreactor system comprises a clarifier container comprising a clarifier working fluid.

203. The method of claim 202, further comprising separating a supernatant portion of the clarifier working fluid from a floc portion of the clarifier working fluid within the clarifier container.
